(19) United States
(12) Patent Application Publication
(10) Pub. No.: US 2025/0011647 A1
(43) Pub. Date: Jan. 9, 2025

(54) ORGANIC ELECTROLUMINESCENT COMPOUND AND APPLICATION THEREOF

(71) Applicant: NINGBO LUMILAN ADVANCED MATERIALS CO., LTD., Ningbo (CN)

(72) Inventors: Ting-Wei WEI, Ningbo (CN); Cong TANG, Ningbo (CN); Qinghua ZOU, Ningbo (CN); Fengjiao LIU, Ningbo (CN); Binyu XU, Ningbo (CN); Xiaoyun XU, Ningbo (CN); Xiaoxin RAN, Ningbo (CN); Renzhou WANG, Ningbo (CN); Qixiang SONG, Ningbo (CN); Huanda DING, Ningbo (CN); Zhi-Kuan CHEN, Ningbo (CN)

(73) Assignee: NINGBO LUMILAN ADVANCED MATERIALS CO., LTD., Ningbo (CN)

(21) Appl. No.: 18/587,693

(22) Filed: Feb. 26, 2024

(30) Foreign Application Priority Data
Jun. 30, 2023 (CN) 202310803717.9
Nov. 29, 2023 (CN) 202311638680.5

(51) Int. Cl. C09K 11/06; H10K 50/11; H10K 50/15; H10K 85/60

(52) U.S. Cl. CPC C09K 11/06; H10K 50/11; H10K 50/15; H10K 85/633; C09K 2211/1011

(57) ABSTRACT

The present application relates to the technical field of display, in particular to an organic electroluminescent compound and an application. The present application provides an organic electroluminescent compound has a structure of the following:

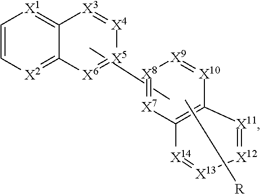

wherein R is selected from the following structure:

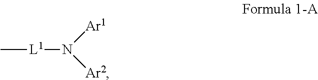

use of organic electroluminescent devices containing this material can result in a more excellent lifespan, as well as lower driving voltage and higher efficiency.

### TECHNICAL FIELD

The present application relates to the technical field of display, in particular to an organic electroluminescent compound and an application thereof.

### BACKGROUND

An organic light emitting device (OLED) converts electrical energy into light by applying electricity to an organic electroluminescent material, and typically includes an anode, a cathode, and an organic layer formed between these two electrodes. The organic layer of the organic electroluminescent device may contain a hole injection layer, a hole transport layer, a hole auxiliary layer, a light emitting auxiliary layer, an electron blocking layer, a light emitting layer (containing a host material and a dopant material), an electron buffer layer, a hole blocking layer, an electron transport layer, an electron injection layer, etc. Based on the functions achieved by each layer, various materials used in the organic layer are divided into hole injection materials, hole transport materials, hole auxiliary materials, light emitting auxiliary materials, electron blocking materials, light emitting materials, electron buffer materials, hole blocking materials, electron transport materials, electron injection materials, etc. In the organic electroluminescent device, holes from the anode and electrons from the cathode are injected into the light emitting layer by applying voltages, and high-energy excitons are produced by the recombination of the holes and the electrons. An organic light emitting compound emits light from energy moving to an excited state and energy at the time when the organic light emitting compound returns to a ground state from the excited state.

In summary, the structural stability of existing organic electroluminescent materials is poor, and the HOMO and LUMO energy levels of existing organic electroluminescent materials are poorly matched with adjacent energy levels, resulting in low stability and unbalanced carrier mobility of organic electroluminescent materials, which leads to problems such as high driving voltage, low luminous efficiency, and short lifespan of organic electroluminescent devices containing this organic electroluminescent material, thereby severely limiting the application of organic electroluminescent devices.

### SUMMARY OF THE INVENTION

The purpose of the present application is to overcome the following problems, the structural stability of existing organic electroluminescent materials is poor, and the HOMO and LUMO energy levels of existing organic electroluminescent materials are poorly matched with adjacent energy levels, resulting in low stability and unbalanced carrier mobility of organic electroluminescent materials, which leads to problems such as high driving voltage, low luminous efficiency, and short lifespan of organic electroluminescent devices containing this organic electroluminescent material, and then provide an organic electroluminescent compound and its applications.

### BRIEF DESCRIPTION OF THE DRAWING

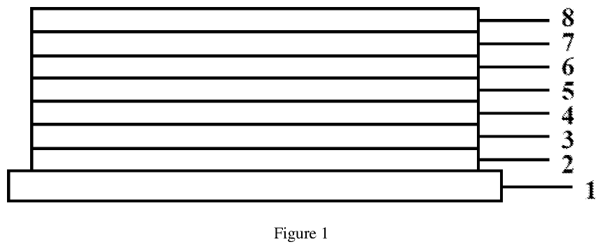

The FIG. 1 illustrates an example organic electroluminescent compound in accordance with one or more embodiments of the present disclosure.

### DETAILED DESCRIPTION

In the present application, the definition of substituent terms are as follows.

In the present application, unless otherwise explicitly stated, the substituents of other various structures are selected from one of or a combination of two of deuterium, halogen, cyano, C1-C6 alkyl, C3-C30 cycloalkyl, C6-C30 aryl, and C3-C30 heteroaryl.

The term “organic electroluminescent material” disclosed in the present application means a material which may be used in an organic electroluminescent device and may contain at least one compound. If necessary, the organic electroluminescent material may be contained in any layer which constitutes the organic electroluminescent device. For example, the organic electroluminescent material may be a hole injection material, a hole transport material, a hole auxiliary material, a light emitting auxiliary material, an electron blocking material, a light emitting material (containing an organic electroluminescent host material and a dopant material), an electron buffer material, a hole blocking material, an electron transport material, an electron injection material, etc.

An organic electroluminescent material disclosed in the present application may contain one organic electroluminescent material or a plurality of organic electroluminescent materials, where the plurality of organic electroluminescent materials refer to materials containing a combination of at least two organic electroluminescent materials, and the materials may be contained in any layer which constitutes the organic electroluminescent device. It may refer to both a material before being contained in the organic electroluminescent device (e.g., before vapor deposition) and a material after being contained in the organic electroluminescent device (e.g., after vapor deposition). For example, the materials may be a combination of at least two compositions, and the compositions may be contained in at least one of the following layers: a hole injection layer, a hole transport layer, a hole auxiliary layer, a light emitting auxiliary layer, an electron blocking layer, a light emitting layer, an electron buffer layer, a hole blocking layer, an electron transport layer and an electron injection layer. The two compositions in the plurality of organic electroluminescent materials may be contained in the same layer or different layers, and may be mixed-evaporated or co-evaporated, or may be evaporated individually.

The term “organic electroluminescent host material composition” disclosed in the present application refers to an organic electroluminescent material containing a combination of at least two host materials. It may refer to both a material before being contained in the organic electroluminescent device (e.g., before vapor deposition) and a material after being contained in the organic electroluminescent device (e.g., after vapor deposition). The composition disclosed in the present application may be contained in any light emitting layer which constitutes the organic electroluminescent device. Two or more compounds contained in the plurality of host materials in the composition disclosed in the present application may be contained in one light emitting layer or may be contained in different light emitting layers respectively. For example, when one layer contains two or more host materials, the layer may be formed through mixed evaporation or may be formed through individual co-evaporation.

The “halogen” in the present application may include fluorine, chlorine, bromine or iodine.

The “C1-C30 alkyl” in the present application refers to a univalent substituent derived from linear or branched saturated hydrocarbon with 1 to 30 carbon atoms, and its examples include but are not limited to methyl, ethyl, n-propyl, isopropyl, n-butyl, isobutyl, sec-butyl, tert-butyl, pentyl, isopentyl and hexyl.

The “C3-C30 cycloalkyl” in the present application refers to monocyclic hydrocarbon or polycyclic hydrocarbon with 1 to 30 cycle main chain carbon atoms, and the C3-C30 cycloalkyl may include cyclopropyl, cyclobutyl, cyclopentyl, cyclohexyl, cyclopentylmethyl, cyclohexylmethyl, adamantyl, etc.

The aryl and arylenyl in the present application include monocyclic, polycyclic or fused-ring aryls, and rings may be separated by short non-aromatic units and may contain spiro structures, including but not limited to phenyl, biphenyl, triphenyl, naphthyl, phenanthryl, phenylphenanthryl, binaphthyl, phenylnaphthyl, naphthylphenyl, anthryl, indenyl, triphenylenyl, tetracenyl, pyrenyl, perylenyl, fluorenyl, phenylfluorenyl, diphenylfluorenyl, benzofluorenyl, spirobifluorenyl, chrysenyl, naphthonaphthyl, fluoranthenyl, etc.

The heteroaryl and heteroarylenyl in the present application include monocyclic, polycyclic or fused-ring heteroaryls, rings may be separated by short non-aromatic units, and heteroatoms include nitrogen, oxygen and sulfur. The heteroaryl and heteroarylenyl include but are not limited to furyl, thienyl, pyrrolyl, imidazolyl, pyrazolyl, thiazolyl, thiadiazolyl, isothiazolyl, isoxazolyl, oxazolyl, oxadiazolyl, triazinyl, tetrazinyl, triazolyl, tetrazolyl, furazanyl, pyridyl, pyrazinyl, pyrimidinyl, pyridazinyl, benzofuryl, benzothienyl, isobenzofuryl, dibenzofuryl, dibenzothienyl, benzimidazolyl, benzothiazolyl, benzoisothiazolyl, benzoisoxazolyl, benzoxazolyl, isoindolyl, indolyl, indazolyl, benzothiadiazolyl, quinolyl, isoquinolyl, cinnolinyl, quinazolinyl, quinoxalinyl, carbazolyl, phenoxazinyl, phenothiazinyl, phenanthridinyl, benzodioxolyl, dihydroacridinyl, and derivatives thereof, etc.

The “substituted” in the present application refers to the substitution of a hydrogen atom in a compound by another substituent. The position is not limited to a specific position, as long as hydrogen(s) at that position can be substituted by substituent(s). It also includes the substitution of the hydrogen atom by a group formed by the connection of two or more substituents. When two or more substituents appear, they can be the same or different. For example, the group formed by the connection of the two or more substituents may be pyridine-triazine group. That is, pyridine-triazine group may be interpreted as a heteroaryl substituent, or a substituent where two heteroaryl substituents are connected.

In the present application, unless otherwise specified, hydrogen atoms include protium, deuterium, and tritium.

The groups of the present application limit the range of carbon atom numbers, which should be any integer within the limited range, for example, C6-C30 aryl represents that the carbon atom number of aryl may be any integer within the range of 6-30, such as 6, 8, 10, 13, 15, 17, 20, 22, 25, or 30, etc.

When the groups in the present application have substituents, the substituents are each independently selected from deuterium, halogen, cyano, nitryl, unsubstituted or R′ substituted C1-C4 linear or branched alkyl, unsubstituted or R′ substituted C6-C20 aryl, unsubstituted or R′ substituted C3-C20 heteroaryl, and unsubstituted or R′ substituted C6-C20 arylamine group; and R′ is selected from deuterium, halogen, cyano and nitryl.

The present application provides an organic electroluminescent compound, wherein the organic electroluminescent compound has a structure of the following Formula (1):

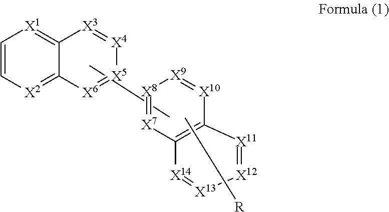

R is selected from the following structure:

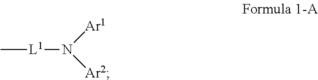

wherein, X¹-X¹⁴ are each independently selected from N or CR⁸, and R⁸ is selected from hydrogen or deuterium;
L¹ is selected from a connecting bond, substituted or unsubstituted C6-C30 arylenyl, and substituted or unsubstituted C3-C30 heteroarylenyl;
Ar¹ and Ar² are each independently selected from substituted or unsubstituted C6-C30 aryl, substituted or unsubstituted C3-C30 heteroaryl, substituted or unsubstituted C6-C60 arylamine group, and substituted or unsubstituted C3-C60 heteroarylamine group;
the substituents in the substituted C6-C30 arylenyl, the substituted C3-C30 heteroarylenyl, the substituted C6-C30 aryl, the substituted C3-C30 heteroaryl, the substituted C6-C60 arylamine group, and the substituted C3-C60 heteroarylamine group are selected from one of or a combination of two of deuterium, halogen, cyano, C1-C6 alkyl, C3-C30 cycloalkyl, C6-C30 aryl, C3-C30 heteroaryl, C6-C60 arylamine group and C3-C60 heteroarylamine group.

It can be understood that the organic electroluminescent compound in the present application has the following structure:

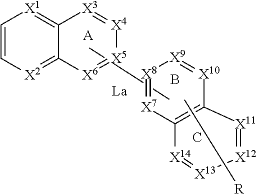

R can be substituted on ring B or on ring C; ring A and ring B are connected through La.

Preferably, in Formula (1), X¹-X¹⁴ are all selected from CR⁸, R⁸ is selected from hydrogen or deuterium;
preferably, X¹-X¹⁴ are all selected from CR⁸, wherein R⁸ is hydrogen;
preferably, any one of X¹-X⁶ is selected from N, and the remaining is CR⁸;
preferably, any one of X¹-X⁶ is selected from N, and the remaining is CR⁸; any one of X⁷-X¹⁴ is selected from N, and the remaining is CR⁸;
wherein R⁸ is each present independently, and can be the same or different; R⁸ is selected from hydrogen or deuterium; and
preferably, Ar¹-Ar² are each independently selected from substituted or unsubstituted A group;
the A group comprises: phenyl, naphthyl, biphenyl, triphenyl, phenanthryl, fluoranthenyl, triphenylenyl, fluorenyl, dimethylfluorenyl, diphenylfluorenyl, spirobifluorenyl, benzodimethylfluorenyl, benzodiphenylfluorenyl, benzospirobifluorenyl, benzofuryl, dibenzofuryl, naphthobenzofuryl, dinaphthalofuryl, benzothienyl, dibenzothienyl, naphthobenzothienyl, carbazolyl, phenylcarbazolyl, benzomethylcarbazolyl, dibenzocarbazolyl, biphenylcarbazolyl, phenanthrobenzofuryl, dibenzofuranofuryl, and phenylcarbazolobenzofuryl;
wherein, the substituent of the substituted A group is selected from one of or a combination of two of deuterium, halogen, cyano, C1-C6 alkyl, C3-C30 cycloalkyl, C6-C30 aryl, C3-C30 heteroaryl, C6-C60 arylamine group, and C3-C60 heteroarylamine group; and
preferably, L¹ is selected from a connecting bond, and substituted or unsubstituted C6-C18 arylenyl.
preferably, Ar¹-Ar² are each independently selected from phenyl, naphthyl, biphenyl, triphenyl, phenanthryl, fluoranthenyl, triphenylenyl, fluorenyl, dimethylfluorenyl, diphenylfluorenyl, spirobifluorenyl, benzodimethylfluorenyl, benzodiphenylfluorenyl, benzospirobifluorenyl, dibenzofuryl, naphthobenzofuryl, dibenzothienyl, naphthobenzothienyl, phenylcarbazolyl, benzophenylcarbazolyl, dibenzophenylcarbazolyl, or a group with the following structure:

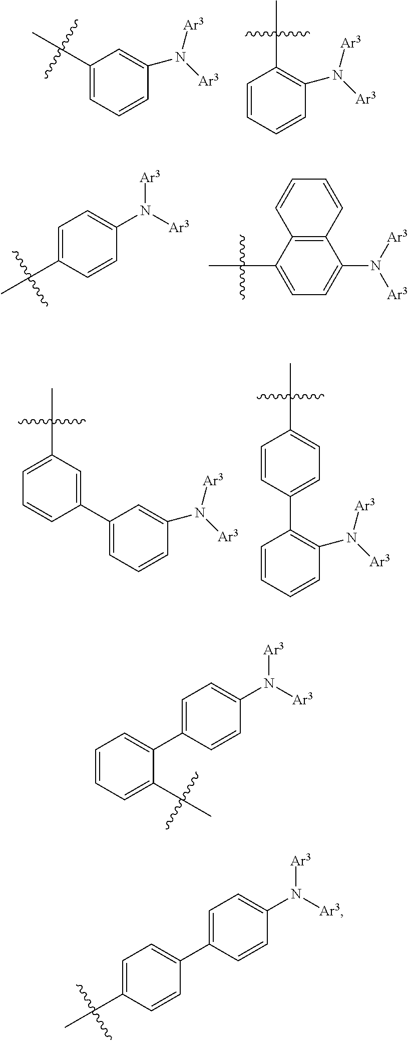

[symbol] represents a single bond connected to adjacent atoms, when a plurality of Ar³ are each present independently, the plurality of Ar³ can be the same or different;
wherein, Ar³ is each independently selected from substituted or unsubstituted B group, and the B group comprises: phenyl, naphthyl, biphenyl, triphenyl, phenanthryl, fluoranthenyl, triphenylenyl, fluorenyl, dimethylfluorenyl, diphenylfluorenyl, spirobifluorenyl, benzodimethylfluorenyl, benzodiphenylfluorenyl, benzospirobifluorenyl, benzofuryl, dibenzofuryl, naphthobenzofuryl, benzothienyl, dibenzothienyl, naphthobenzothienyl, carbazolyl, phenylcarbazolyl, benzophenylcarbazolyl, and dibenzophenylcarbazolyl;
wherein, the substituent of the substituted B group is selected from one of or a combination of two of deuterium, halogen, cyano, C1-C6 alkyl, C3-C30 cycloalkyl, C6-C30 aryl, C3-C30 heteroaryl, C6-C60 arylamine group, and C3-C60 heteroarylamine group; and
preferably, L¹ is selected from a connecting bond, phenylenyl and naphthenyl.

Preferably, the structure of the organic electroluminescent compound is shown as any one of Formula 1-1 to Formula 1-64:

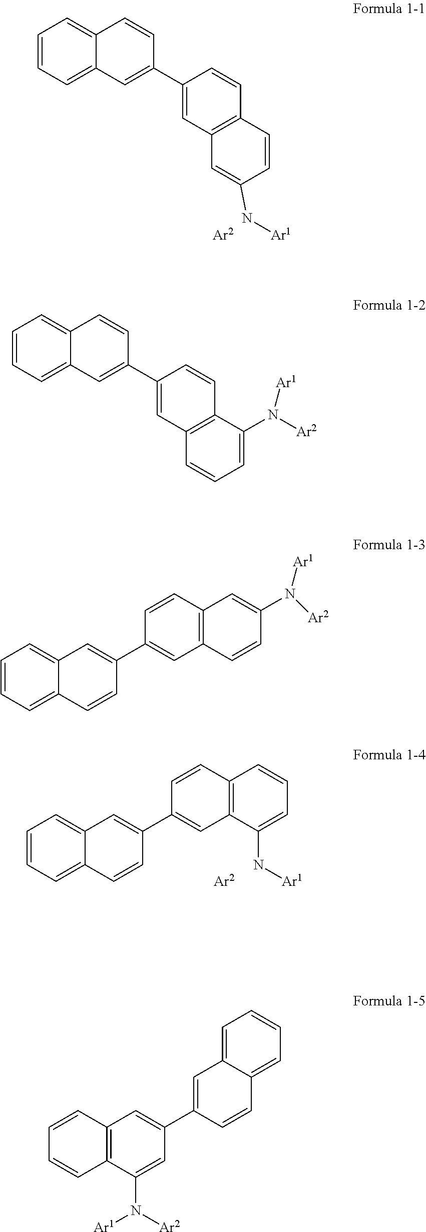

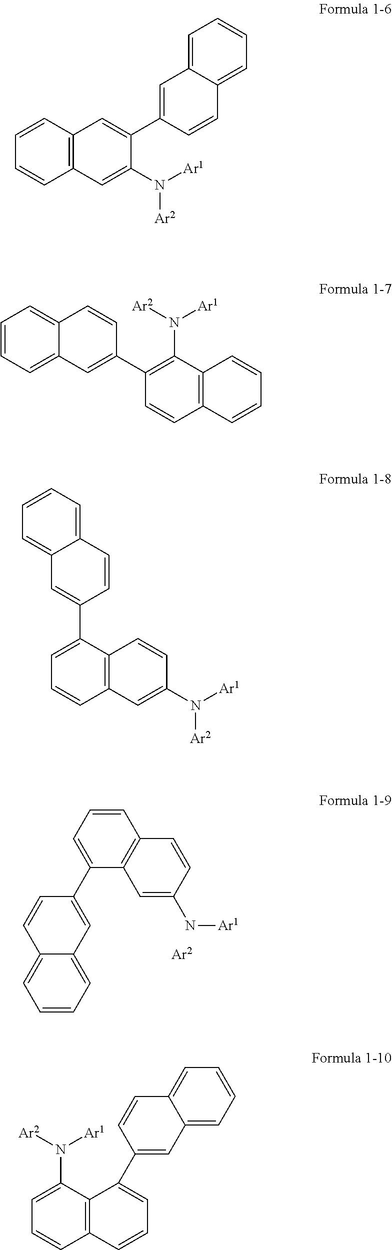

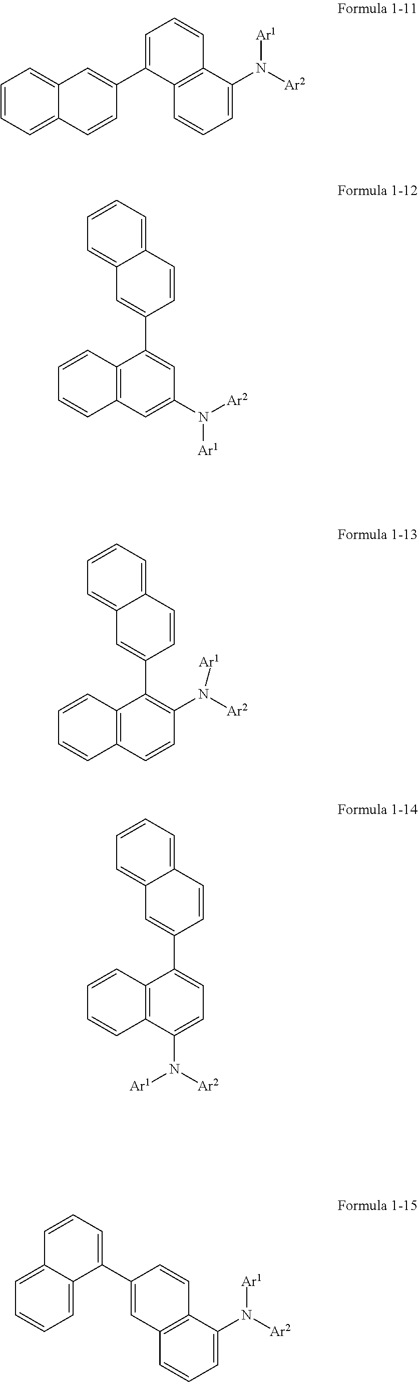

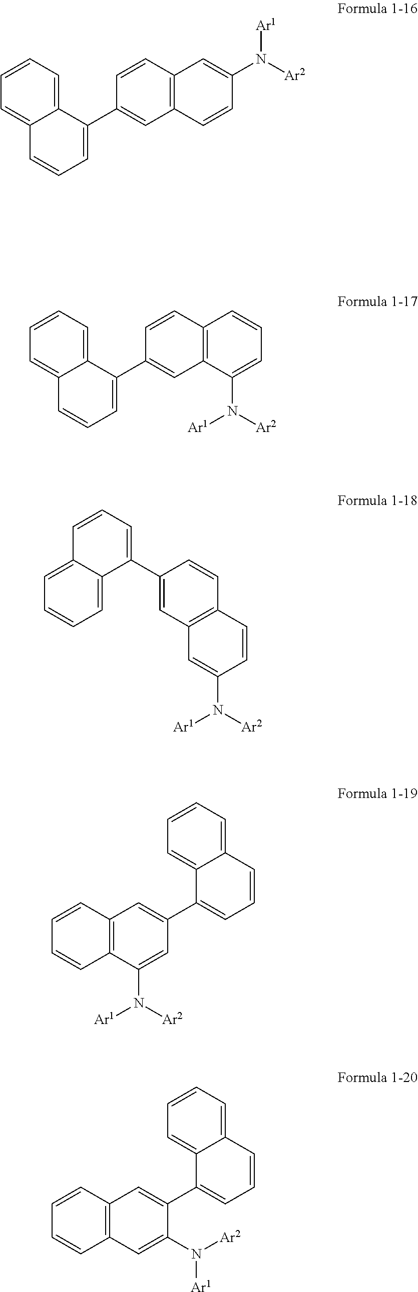

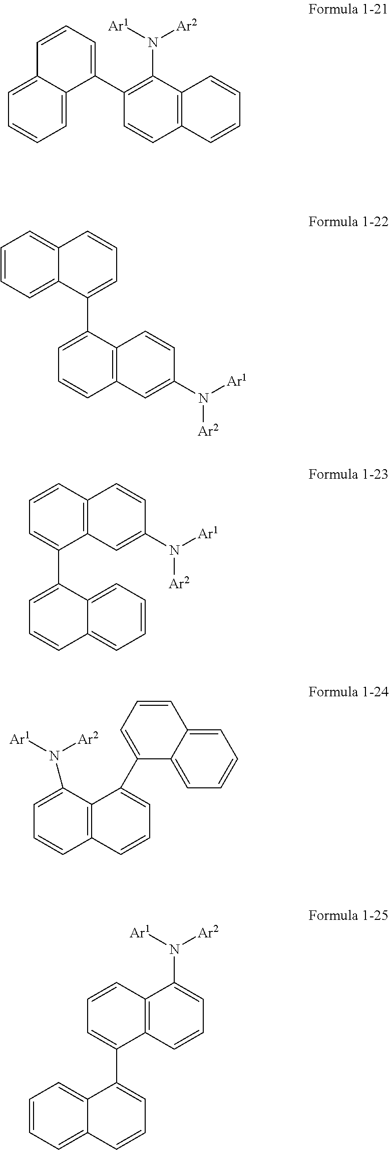

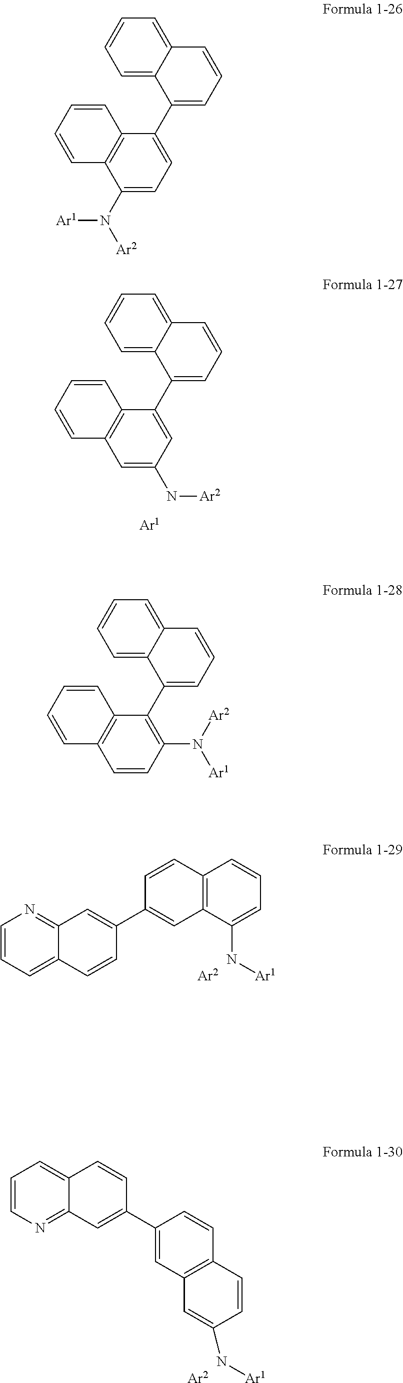

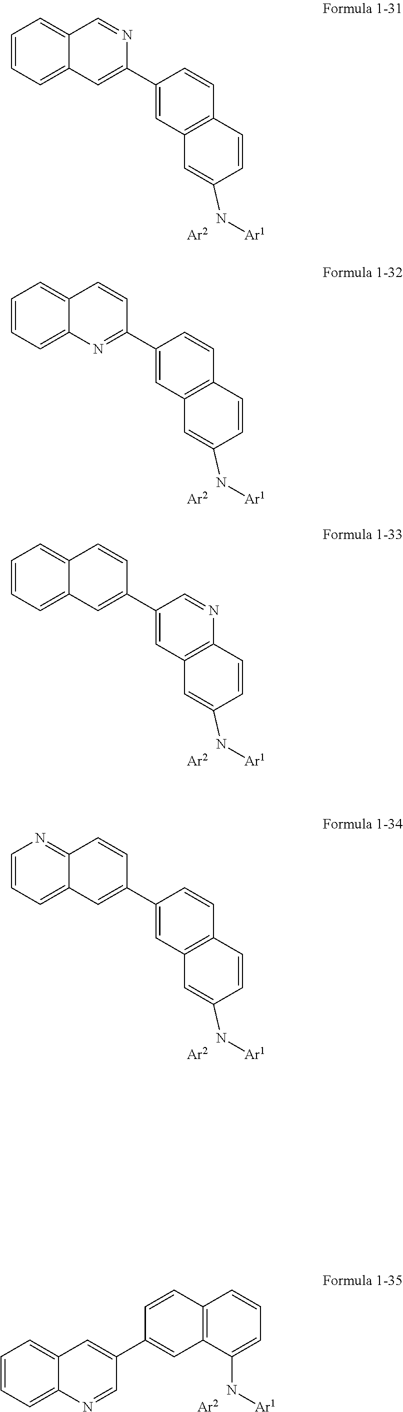

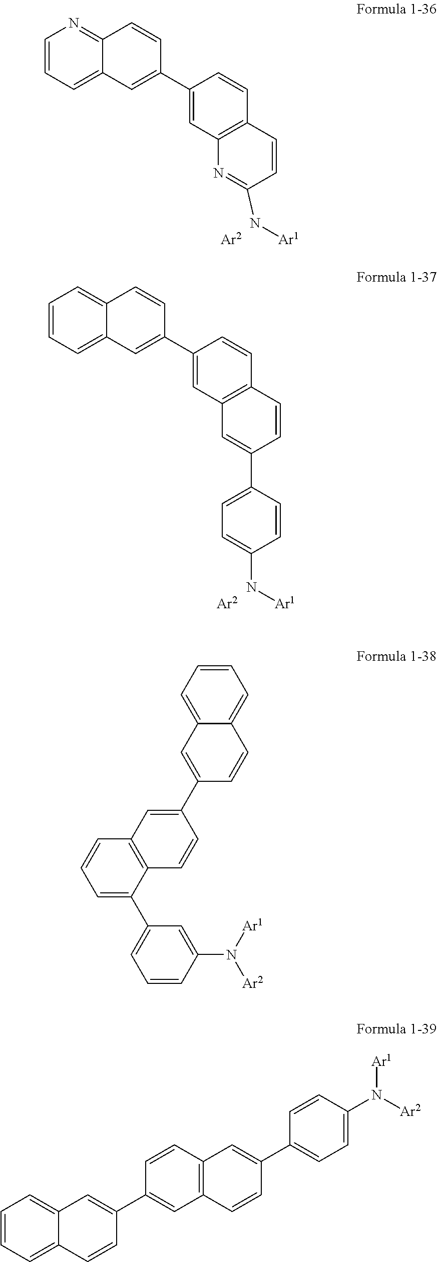

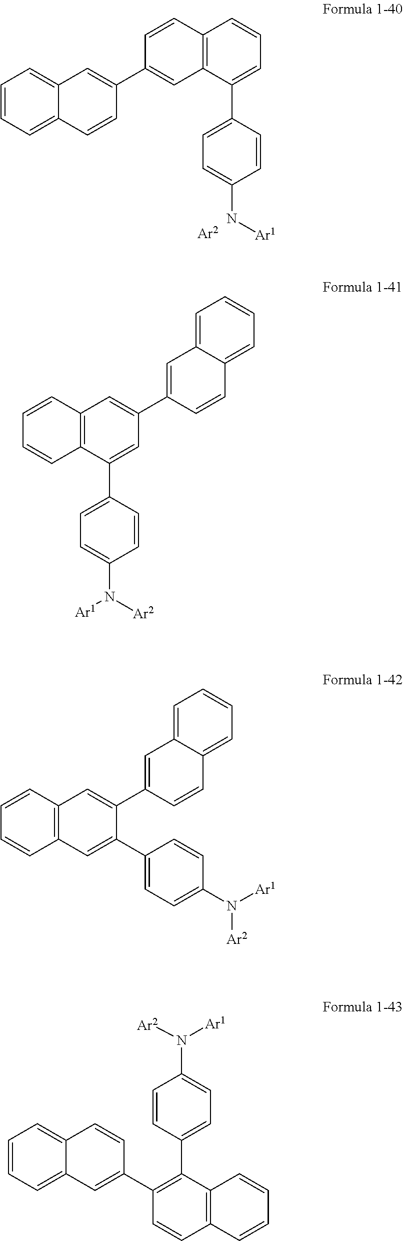

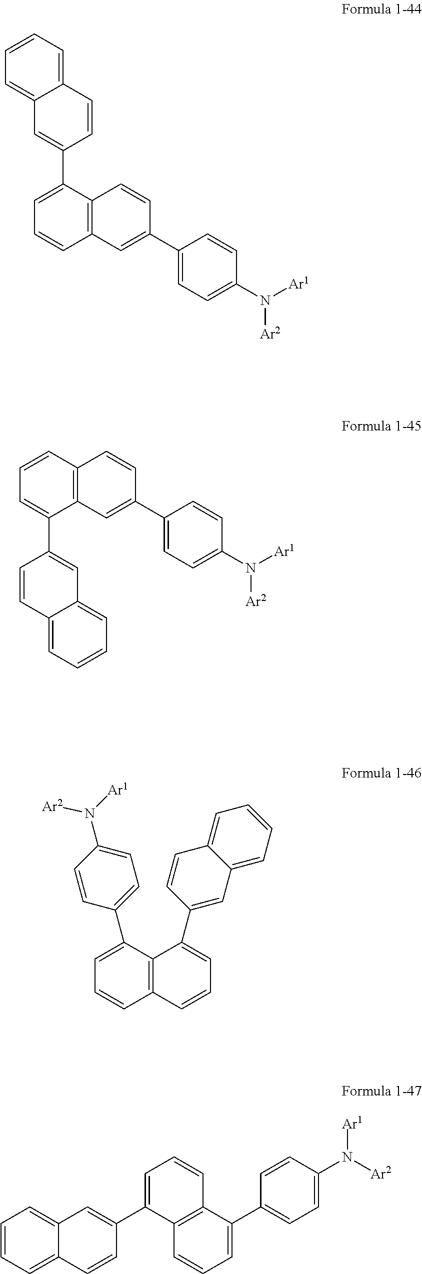

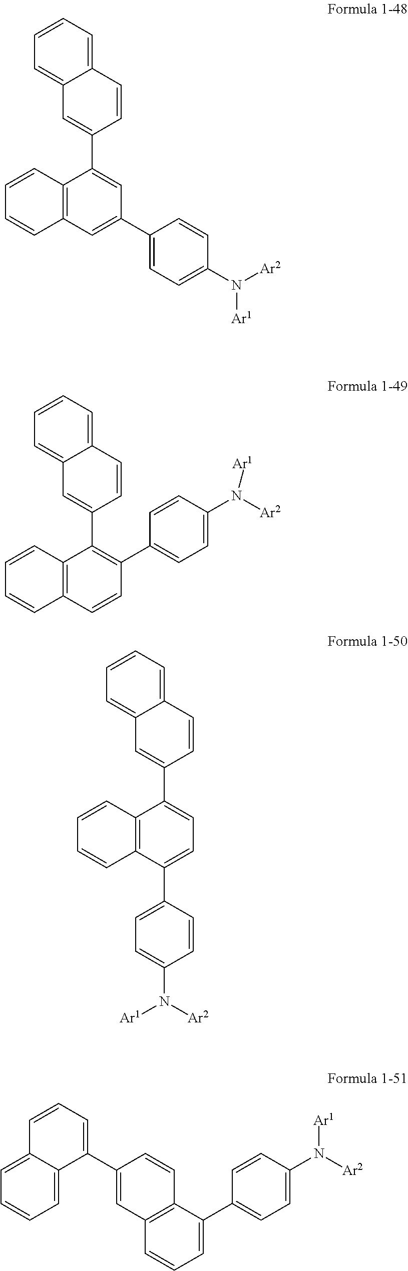

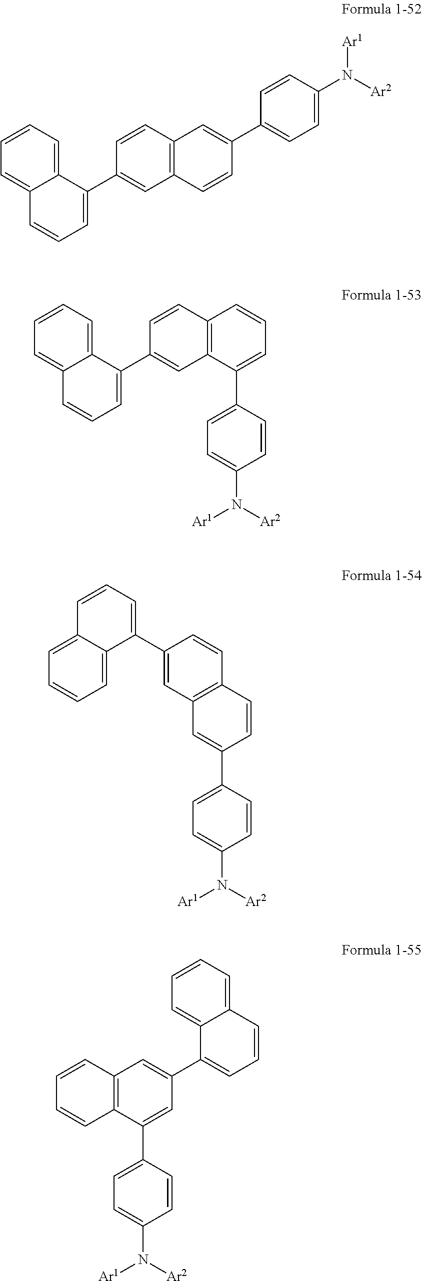

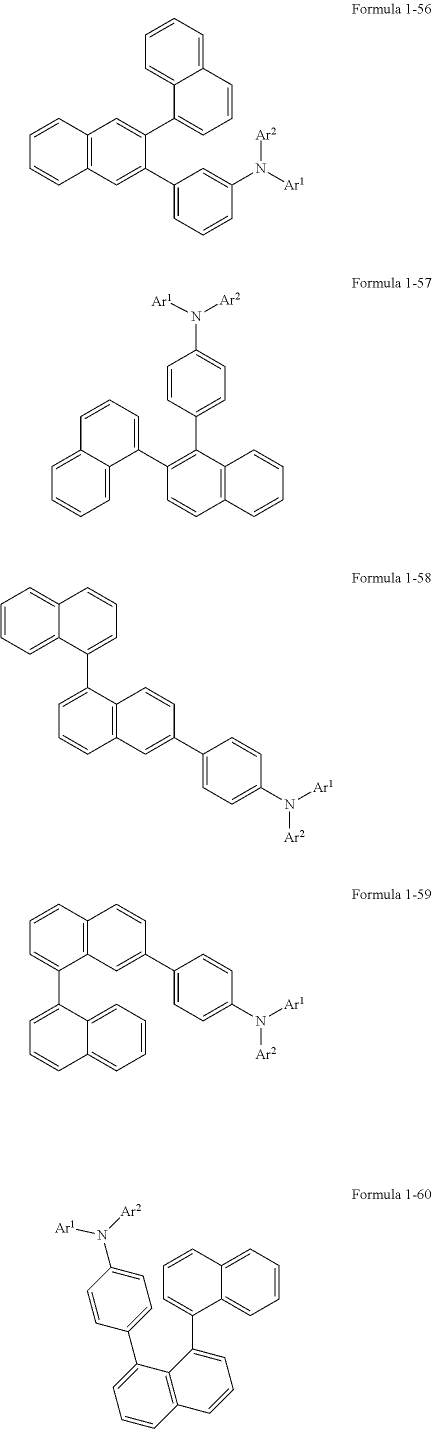

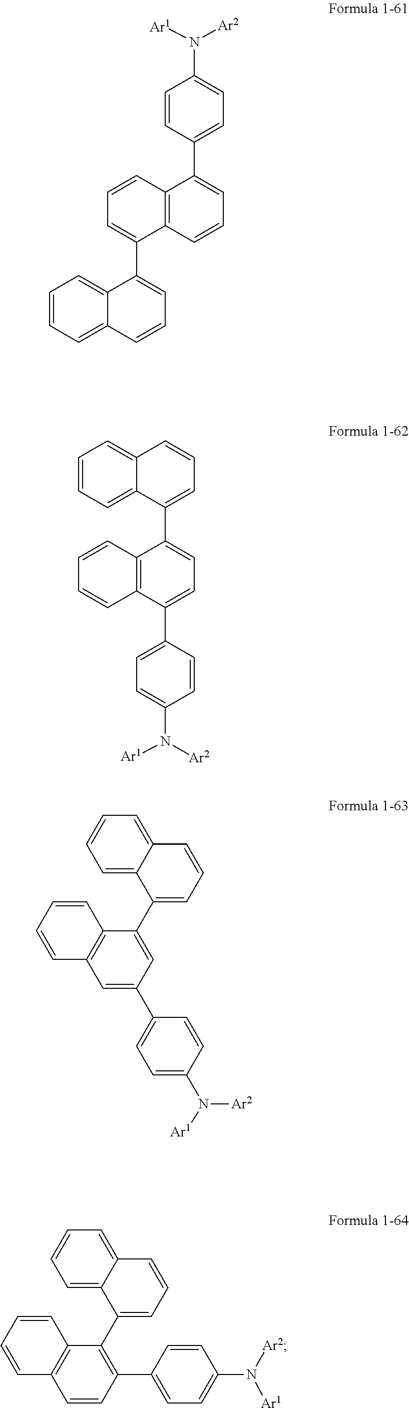

wherein Ar¹-Ar² are defined the same as the above.
wherein in Formula 1-A, Ar² is selected from the following groups:

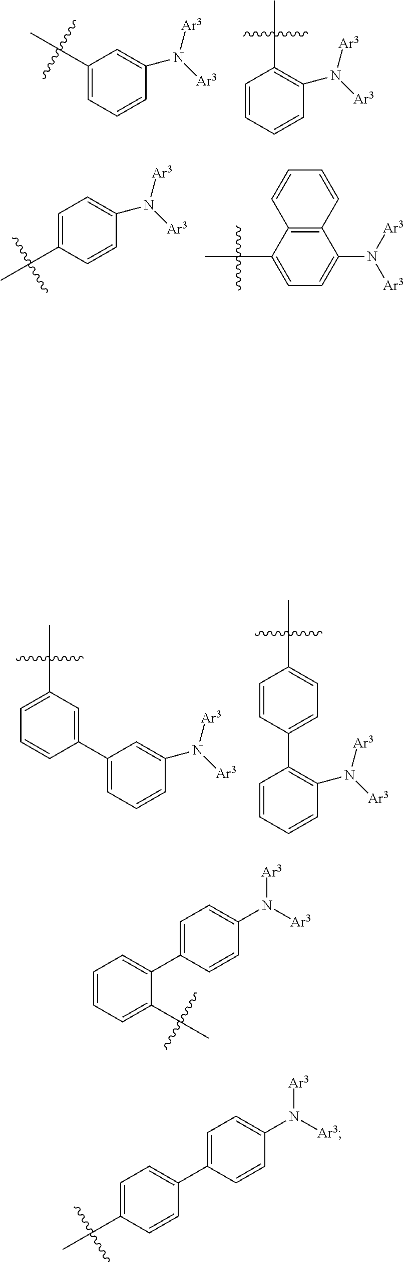

wherein Ar³ is defined the same as the above.

Preferably, the organic electroluminescent compound has a structure as shown in any one of Formula 1-a to Formula 1-h:

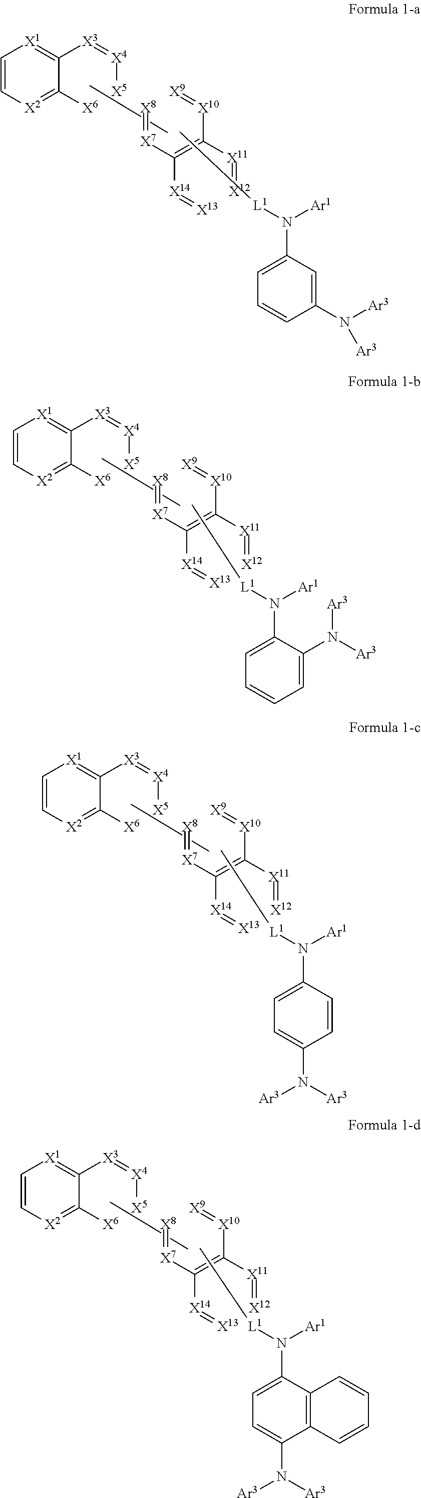

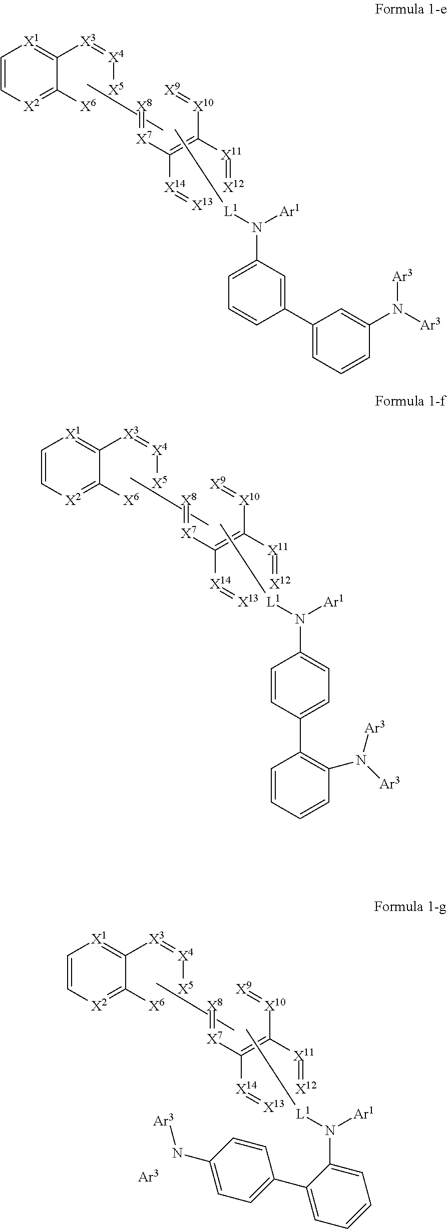

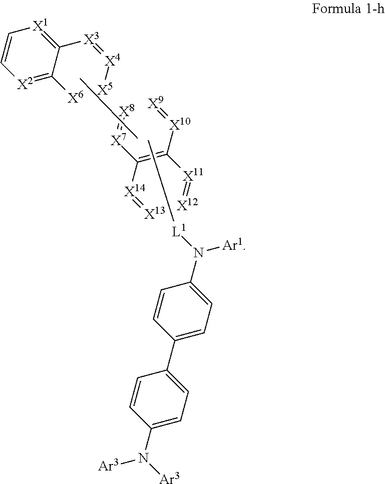

Preferably, wherein the organic electroluminescent compound has a structure as shown in any one of Formula 1-i or Formula 1-ii:

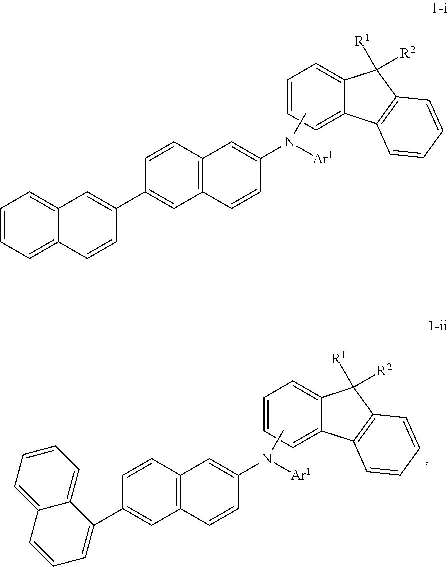

wherein Ar¹ is defined the same as the above; R¹-R² are each independently selected from one of or a combination of two of hydrogen, deuterium, substituted or unsubstituted C1-C6 alkyl, substituted or unsubstituted C3-C30 cycloalkyl, substituted or unsubstituted C6-C30 aryl, and substituted or unsubstituted C3-C30 heteroaryl;
the substituent in the substituted C1-C6 alkyl, the substituted C3-C30 cycloalkyl, the substituted C6-C30 aryl, and the substituted C3-C30 heteroaryl is selected from one of or a combination of two of deuterium, halogen, cyano, C1-C6 alkyl, C3-C30 cycloalkyl, C6-C30 aryl, C3-C30 heteroaryl, C6-C60 arylamine group, and C3-C60 heteroarylamine group.

Preferably, in Formula 1-i or Formula 1-ii, R¹-R² are each independently selected from C3-C15 cycloalkyl and C6-C15 aryl;
preferably, R¹ and R² are each independently selected from methyl, phenyl, and fluorenyl;
preferably, Ar¹ is selected from substituted or unsubstituted C6-C15 aryl and substituted or unsubstituted C3-C15 heteroaryl; wherein, the substituent of the substituted C6-C15 aryl and the substituted C3-C15 heteroaryl is selected from deuterium, C1-C3 alkyl, C3-C10 cycloalkyl, and C6-C12 aryl;
preferably, Ar¹ is selected from tolyl, phenyl, biphenyl, and dibenzofuryl.

Preferably, the organic electroluminescent compound has a structure as shown in any one of Formula N-i-1 to Formula N-i-30:

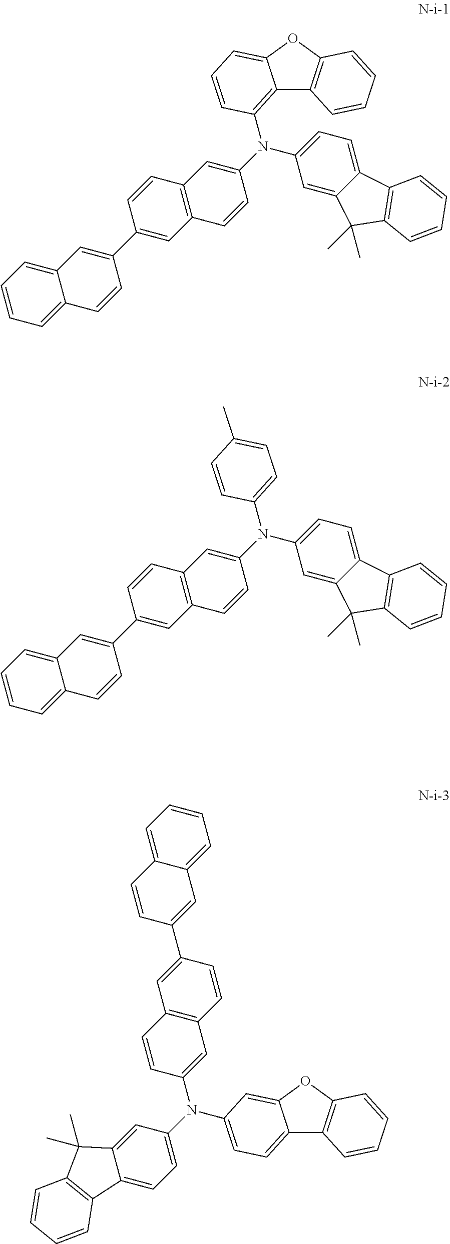 [SMILES: CC1(C)c2ccccc2-c2ccc(N(c3ccc4cc(-c5ccc6ccccc6c5)ccc4c3)c3cccc4oc5ccccc5c34)cc21 ; Cc1ccc(N(c2ccc3c(c2)C(C)(C)c2ccccc2-3)c2ccc3cc(-c4ccc5ccccc5c4)ccc3c2)cc1 ; CC1(C)c2ccccc2-c2ccc(N(c3ccc4cc(-c5ccc6ccccc6c5)ccc4c3)c3ccc4c(c3)oc3ccccc34)cc21]

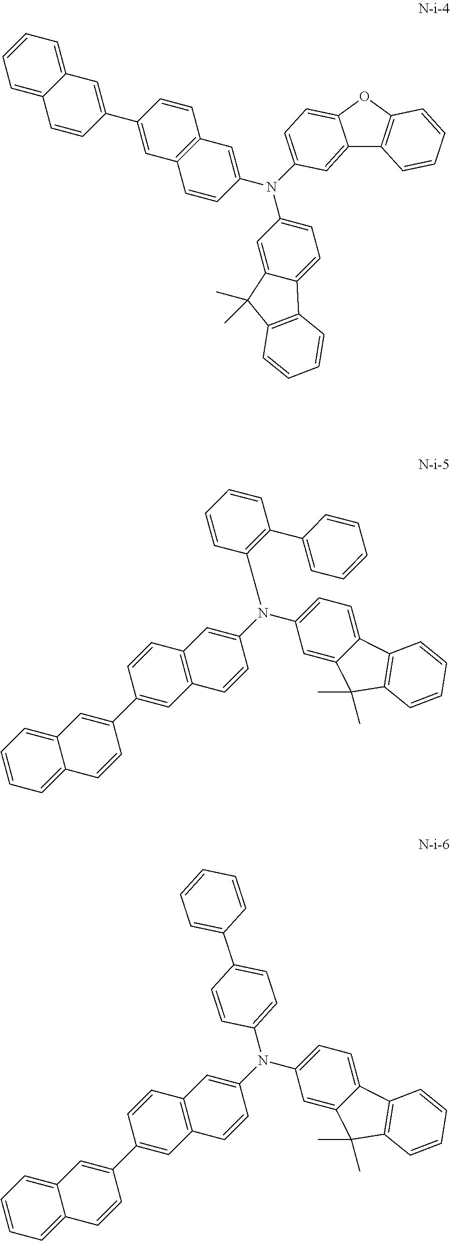 [SMILES: CC1(C)c2ccccc2-c2ccc(N(c3ccc4cc(-c5ccc6ccccc6c5)ccc4c3)c3ccc4oc5ccccc5c4c3)cc21 ; CC1(C)c2ccccc2-c2ccc(N(c3ccc4cc(-c5ccc6ccccc6c5)ccc4c3)c3ccccc3-c3ccccc3)cc21 ; CC1(C)c2ccccc2-c2ccc(N(c3ccc(-c4ccccc4)cc3)c3ccc4cc(-c5ccc6ccccc6c5)ccc4c3)cc21]

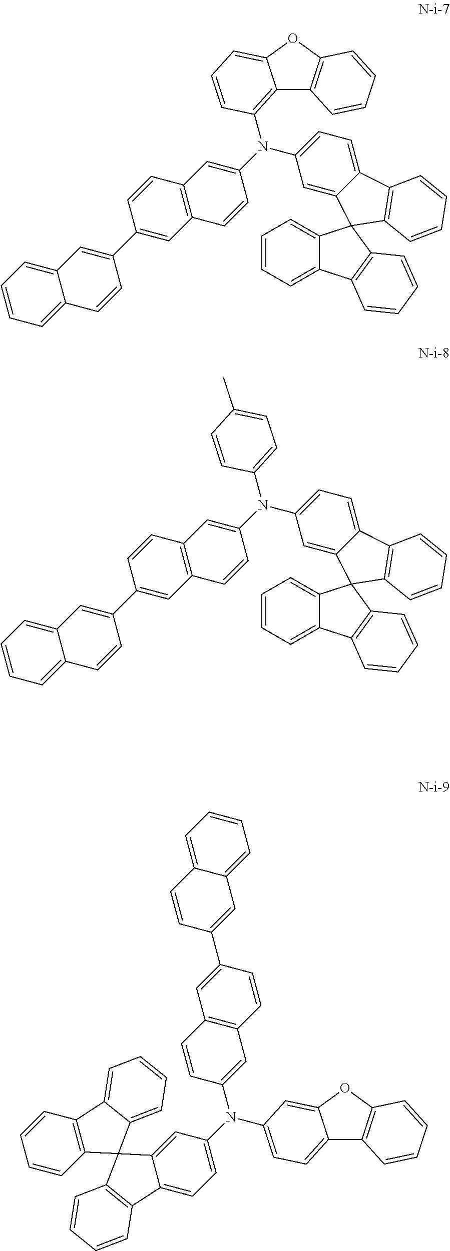 [SMILES: c1ccc2c(c1)-c1ccccc1C21c2ccccc2-c2ccc(N(c3ccc4cc(-c5ccc6ccccc6c5)ccc4c3)c3cccc4oc5ccccc5c34)cc21 ; Cc1ccc(N(c2ccc3c(c2)C2(c4ccccc4-c4ccccc42)c2ccccc2-3)c2ccc3cc(-c4ccc5ccccc5c4)ccc3c2)cc1 ; c1ccc2c(c1)-c1ccccc1C21c2ccccc2-c2ccc(N(c3ccc4cc(-c5ccc6ccccc6c5)ccc4c3)c3ccc4c(c3)oc3ccccc34)cc21]

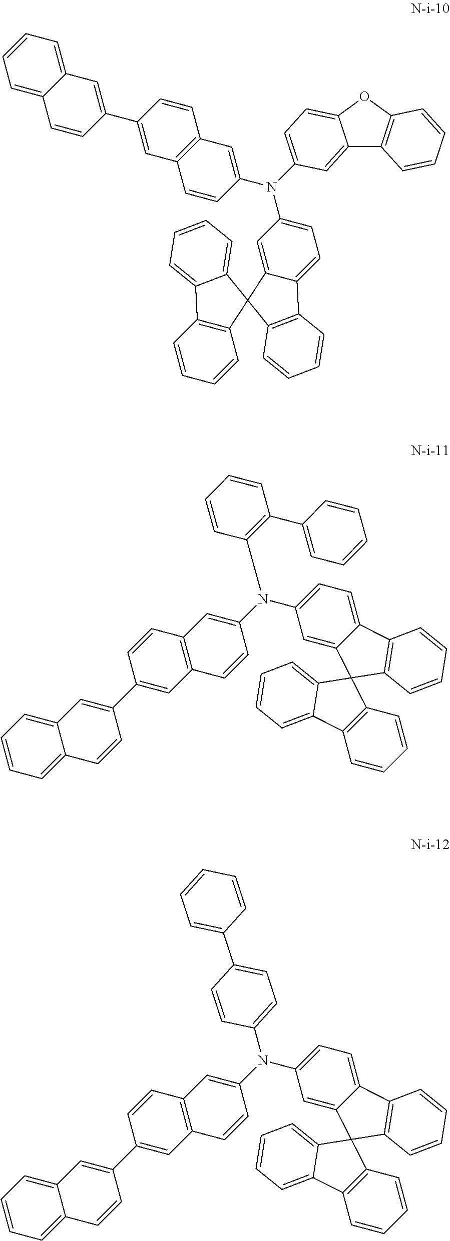 [SMILES: c1ccc2c(c1)-c1ccccc1C21c2ccccc2-c2ccc(N(c3ccc4cc(-c5ccc6ccccc6c5)ccc4c3)c3ccc4oc5ccccc5c4c3)cc21 ; c1ccc(-c2ccccc2N(c2ccc3c(c2)C2(c4ccccc4-c4ccccc42)c2ccccc2-3)c2ccc3cc(-c4ccc5ccccc5c4)ccc3c2)cc1 ; c1ccc(-c2ccc(N(c3ccc4c(c3)C3(c5ccccc5-c5ccccc53)c3ccccc3-4)c3ccc4cc(-c5ccc6ccccc6c5)ccc4c3)cc2)cc1]

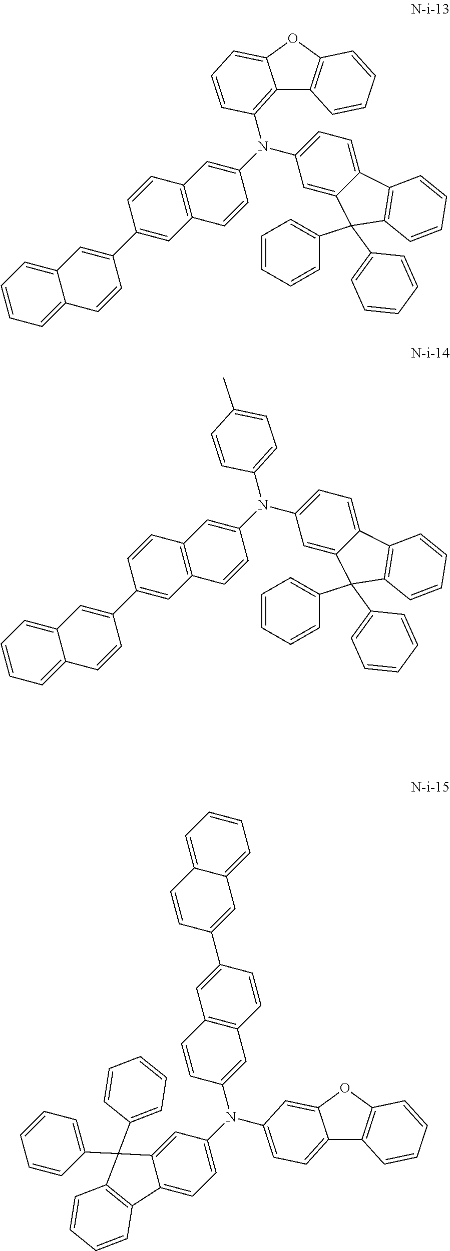 [SMILES: c1ccc(C2(c3ccccc3)c3ccccc3-c3ccc(N(c4ccc5cc(-c6ccc7ccccc7c6)ccc5c4)c4cccc5oc6ccccc6c45)cc32)cc1 ; Cc1ccc(N(c2ccc3c(c2)C(c2ccccc2)(c2ccccc2)c2ccccc2-3)c2ccc3cc(-c4ccc5ccccc5c4)ccc3c2)cc1 ; c1ccc(C2(c3ccccc3)c3ccccc3-c3ccc(N(c4ccc5cc(-c6ccc7ccccc7c6)ccc5c4)c4ccc5c(c4)oc4ccccc45)cc32)cc1]

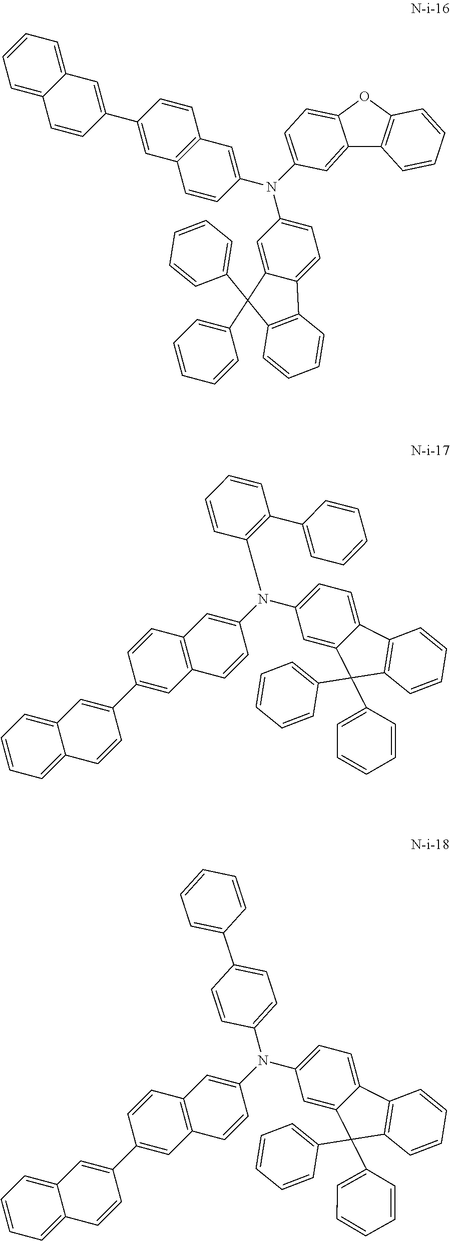 [SMILES: c1ccc(C2(c3ccccc3)c3ccccc3-c3ccc(N(c4ccc5cc(-c6ccc7ccccc7c6)ccc5c4)c4ccc5oc6ccccc6c5c4)cc32)cc1 ; c1ccc(-c2ccccc2N(c2ccc3c(c2)C(c2ccccc2)(c2ccccc2)c2ccccc2-3)c2ccc3cc(-c4ccc5ccccc5c4)ccc3c2)cc1 ; c1ccc(-c2ccc(N(c3ccc4c(c3)C(c3ccccc3)(c3ccccc3)c3ccccc3-4)c3ccc4cc(-c5ccc6ccccc6c5)ccc4c3)cc2)cc1]

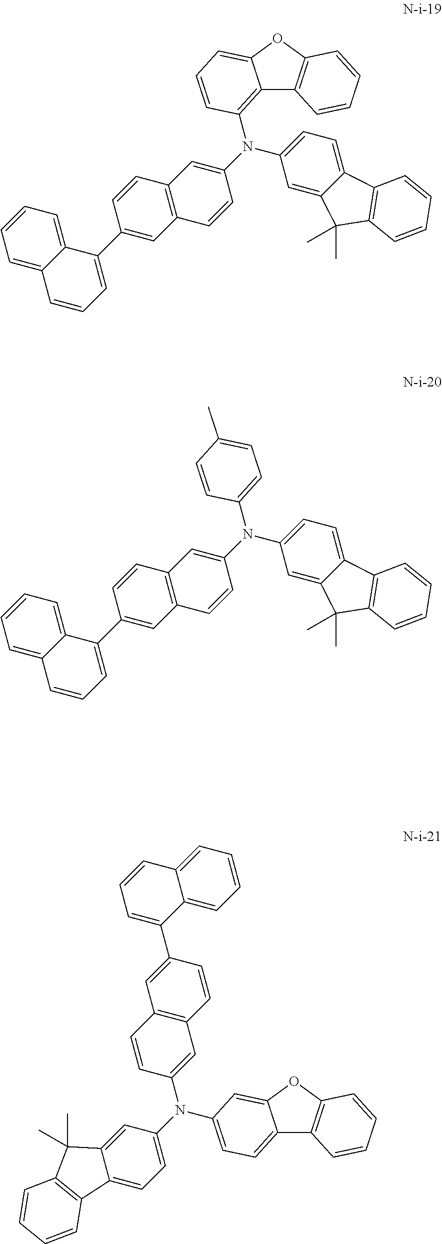 [SMILES: CC1(C)c2ccccc2-c2ccc(N(c3ccc4cc(-c5cccc6ccccc56)ccc4c3)c3cccc4oc5ccccc5c34)cc21 ; Cc1ccc(N(c2ccc3c(c2)C(C)(C)c2ccccc2-3)c2ccc3cc(-c4cccc5ccccc45)ccc3c2)cc1 ; CC1(C)c2ccccc2-c2ccc(N(c3ccc4cc(-c5cccc6ccccc56)ccc4c3)c3ccc4c(c3)oc3ccccc34)cc21]

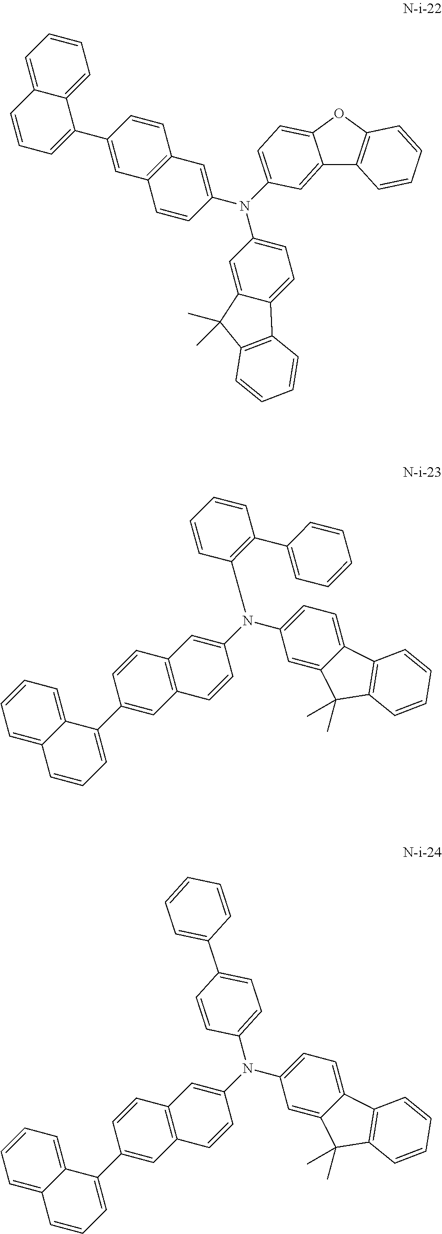 [SMILES: CC1(C)c2ccccc2-c2ccc(N(c3ccc4cc(-c5cccc6ccccc56)ccc4c3)c3ccc4oc5ccccc5c4c3)cc21 ; CC1(C)c2ccccc2-c2ccc(N(c3ccc4cc(-c5cccc6ccccc56)ccc4c3)c3ccccc3-c3ccccc3)cc21 ; CC1(C)c2ccccc2-c2ccc(N(c3ccc(-c4ccccc4)cc3)c3ccc4cc(-c5cccc6ccccc56)ccc4c3)cc21]

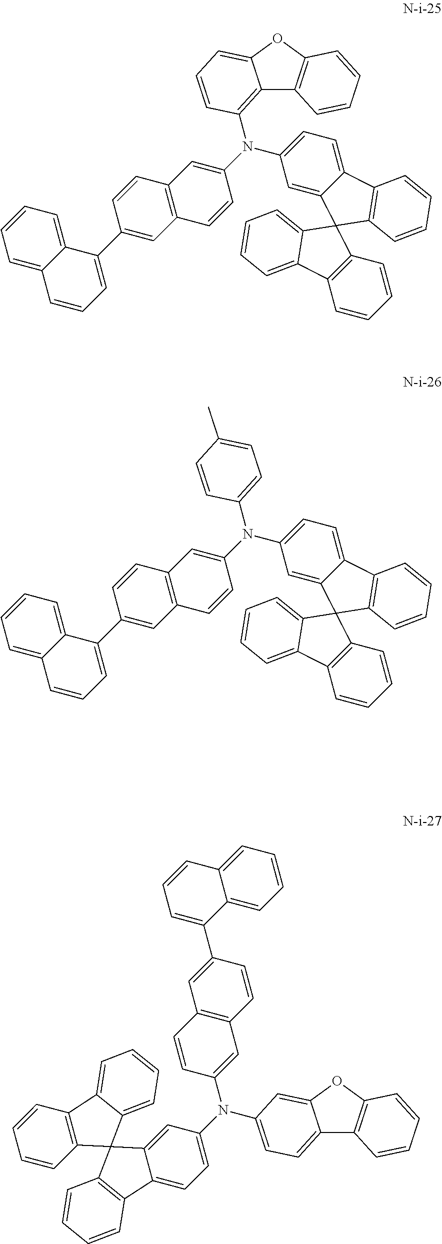 [SMILES: c1ccc2c(c1)-c1ccccc1C21c2ccccc2-c2ccc(N(c3ccc4cc(-c5cccc6ccccc56)ccc4c3)c3cccc4oc5ccccc5c34)cc21 ; Cc1ccc(N(c2ccc3c(c2)C2(c4ccccc4-c4ccccc42)c2ccccc2-3)c2ccc3cc(-c4cccc5ccccc45)ccc3c2)cc1 ; c1ccc2c(c1)-c1ccccc1C21c2ccccc2-c2ccc(N(c3ccc4cc(-c5cccc6ccccc56)ccc4c3)c3ccc4c(c3)oc3ccccc34)cc21]

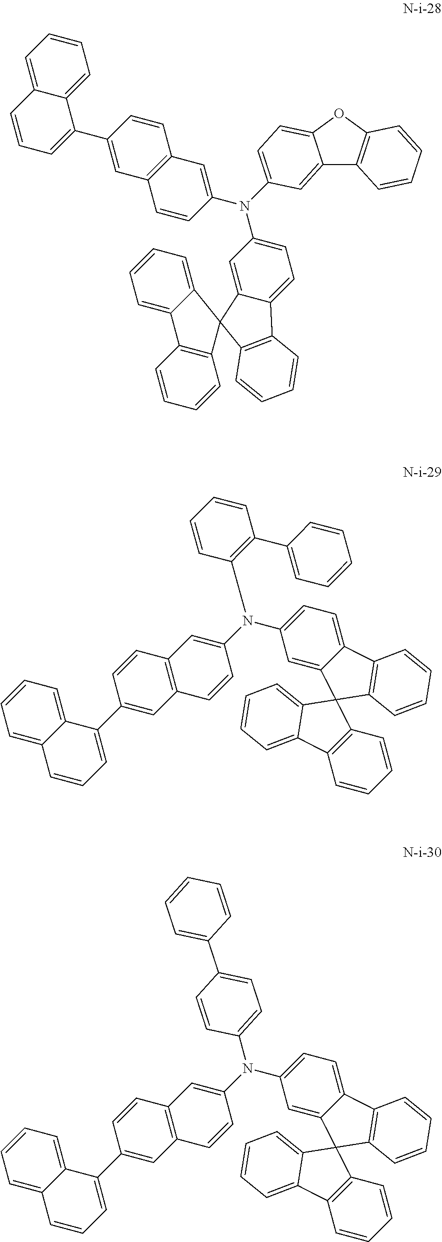 [SMILES: c1ccc2c(c1)-c1ccccc1C21c2ccccc2-c2ccc(N(c3ccc4cc(-c5cccc6ccccc56)ccc4c3)c3ccc4oc5ccccc5c4c3)cc21 ; c1ccc(-c2ccccc2N(c2ccc3c(c2)C2(c4ccccc4-c4ccccc42)c2ccccc2-3)c2ccc3cc(-c4cccc5ccccc45)ccc3c2)cc1 ; c1ccc(-c2ccc(N(c3ccc4c(c3)C3(c5ccccc5-c5ccccc53)c3ccccc3-4)c3ccc4cc(-c5cccc6ccccc56)ccc4c3)cc2)cc1]

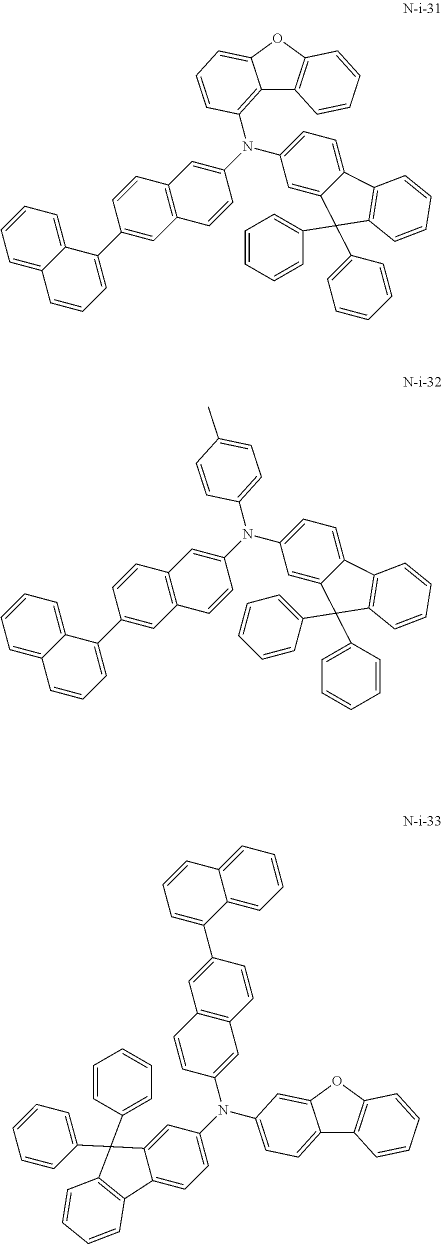 [SMILES: c1ccc(C2(c3ccccc3)c3ccccc3-c3ccc(N(c4ccc5cc(-c6cccc7ccccc67)ccc5c4)c4cccc5oc6ccccc6c45)cc32)cc1 ; Cc1ccc(N(c2ccc3c(c2)C(c2ccccc2)(c2ccccc2)c2ccccc2-3)c2ccc3cc(-c4cccc5ccccc45)ccc3c2)cc1 ; c1ccc(C2(c3ccccc3)c3ccccc3-c3ccc(N(c4ccc5cc(-c6cccc7ccccc67)ccc5c4)c4ccc5c(c4)oc4ccccc45)cc32)cc1]

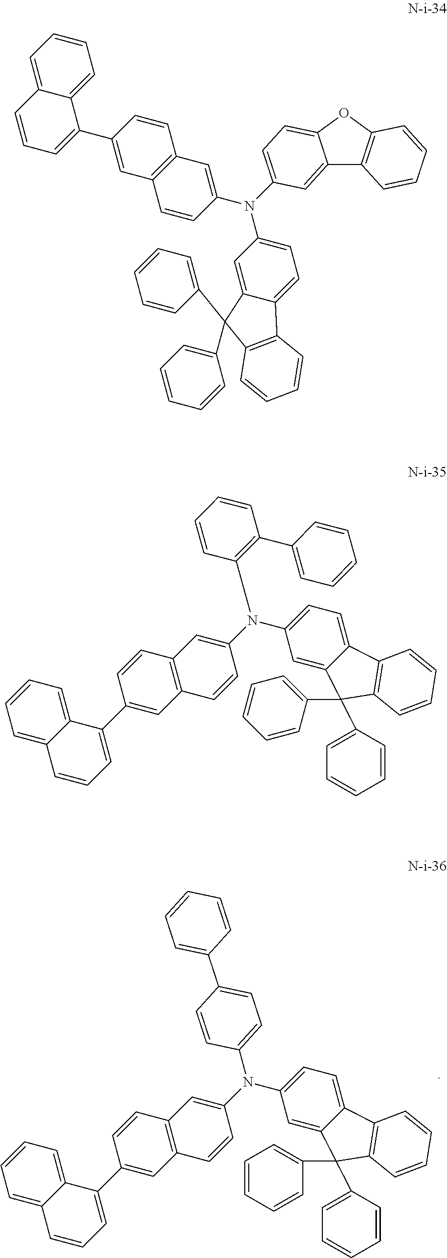 [SMILES: c1ccc(C2(c3ccccc3)c3ccccc3-c3ccc(N(c4ccc5cc(-c6cccc7ccccc67)ccc5c4)c4ccc5oc6ccccc6c5c4)cc32)cc1 ; c1ccc(-c2ccccc2N(c2ccc3c(c2)C(c2ccccc2)(c2ccccc2)c2ccccc2-3)c2ccc3cc(-c4cccc5ccccc45)ccc3c2)cc1 ; c1ccc(-c2ccc(N(c3ccc4c(c3)C(c3ccccc3)(c3ccccc3)c3ccccc3-4)c3ccc4cc(-c5cccc6ccccc56)ccc4c3)cc2)cc1]

Preferably, the organic electroluminescent compound has a structure as shown in any one of N-1 to N-548:

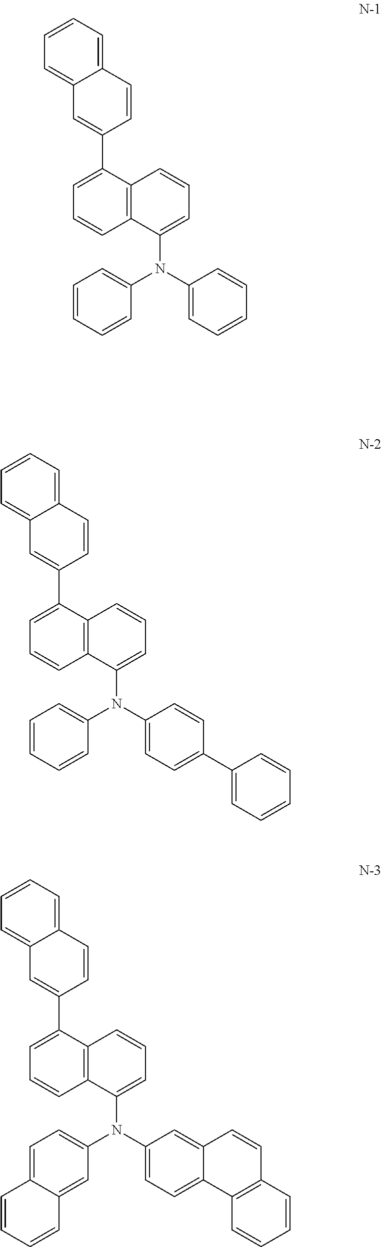 [SMILES: c1ccc(N(c2ccccc2)c2cccc3c(-c4ccc5ccccc5c4)cccc23)cc1 ; c1ccc(-c2ccc(N(c3ccccc3)c3cccc4c(-c5ccc6ccccc6c5)cccc34)cc2)cc1 ; c1ccc2cc(-c3cccc4c(N(c5ccc6ccccc6c5)c5ccc6c(ccc7ccccc76)c5)cccc34)ccc2c1]

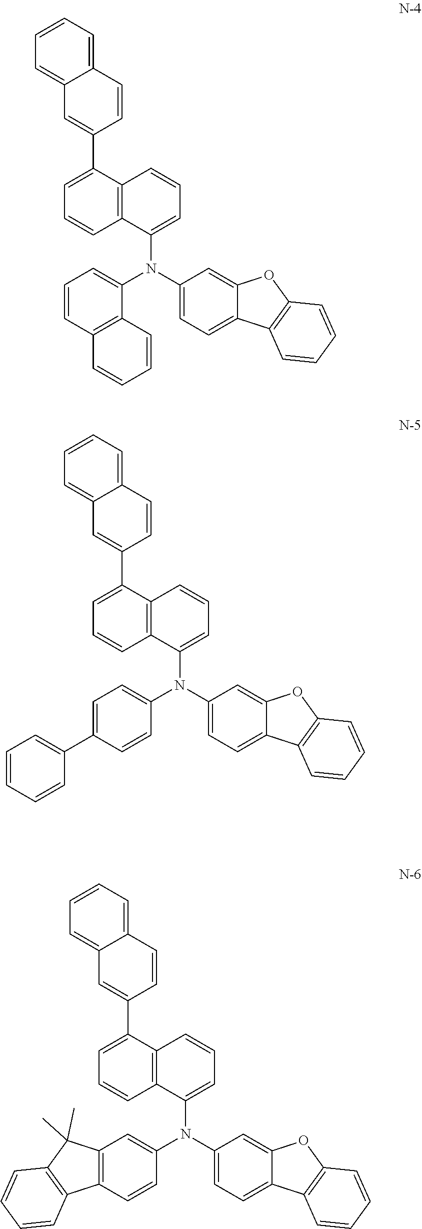 [SMILES: c1ccc2cc(-c3cccc4c(N(c5ccc6c(c5)oc5ccccc56)c5cccc6ccccc56)cccc34)ccc2c1 ; c1ccc(-c2ccc(N(c3ccc4c(c3)oc3ccccc34)c3cccc4c(-c5ccc6ccccc6c5)cccc34)cc2)cc1 ; CC1(C)c2ccccc2-c2ccc(N(c3ccc4c(c3)oc3ccccc34)c3cccc4c(-c5ccc6ccccc6c5)cccc34)cc21]

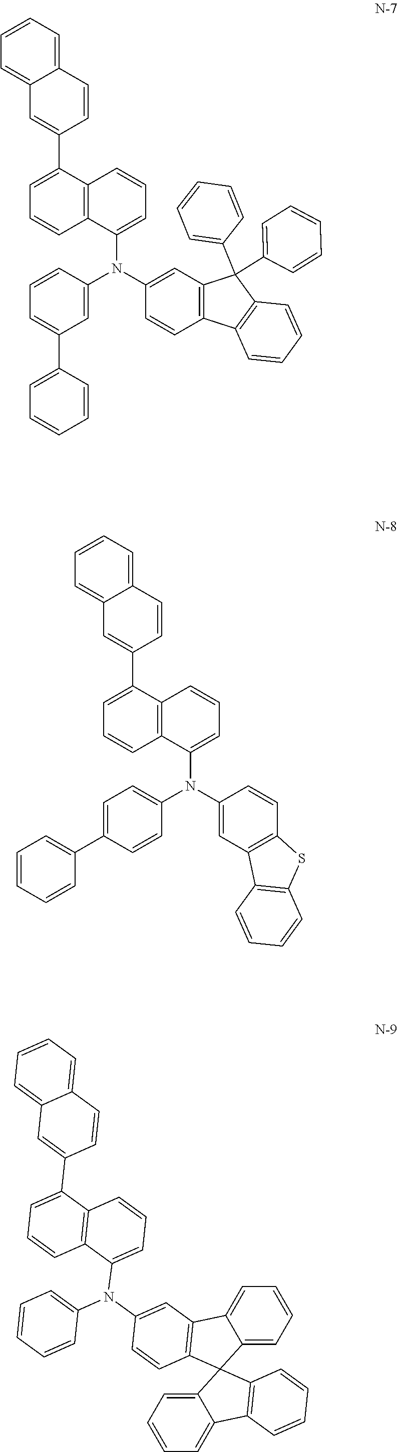 [SMILES: c1ccc(-c2cccc(N(c3ccc4c(c3)C(c3ccccc3)(c3ccccc3)c3ccccc3-4)c3cccc4c(-c5ccc6ccccc6c5)cccc34)c2)cc1 ; c1ccc(-c2ccc(N(c3ccc4sc5ccccc5c4c3)c3cccc4c(-c5ccc6ccccc6c5)cccc34)cc2)cc1 ; c1ccc(N(c2ccc3c(c2)-c2ccccc2C32c3ccccc3-c3ccccc32)c2cccc3c(-c4ccc5ccccc5c4)cccc23)cc1]

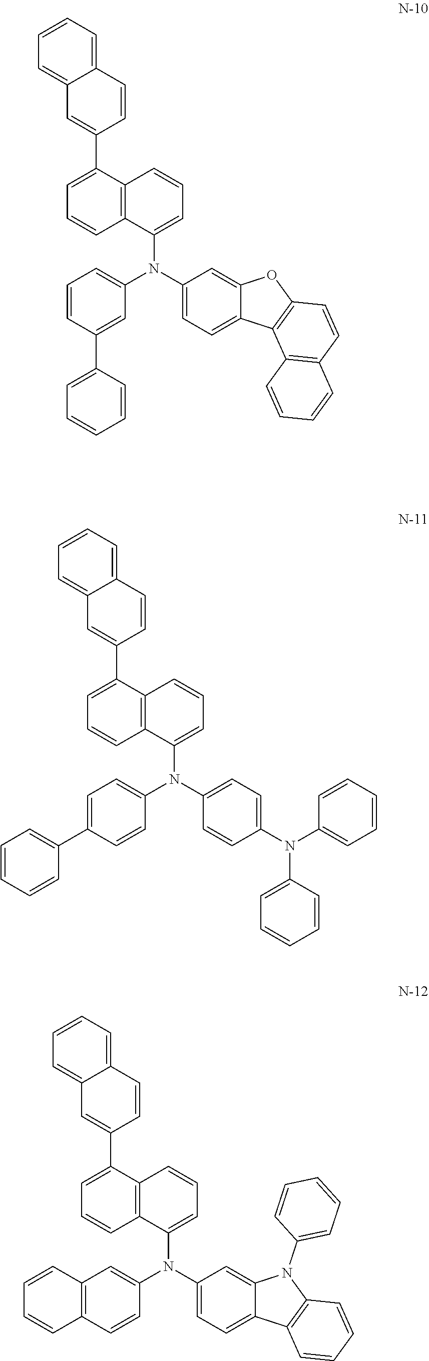 [SMILES: c1ccc(-c2cccc(N(c3ccc4c(c3)oc3ccc5ccccc5c34)c3cccc4c(-c5ccc6ccccc6c5)cccc34)c2)cc1 ; c1ccc(-c2ccc(N(c3ccc(N(c4ccccc4)c4ccccc4)cc3)c3cccc4c(-c5ccc6ccccc6c5)cccc34)cc2)cc1 ; c1ccc(-n2c3ccccc3c3ccc(N(c4ccc5ccccc5c4)c4cccc5c(-c6ccc7ccccc7c6)cccc45)cc32)cc1]

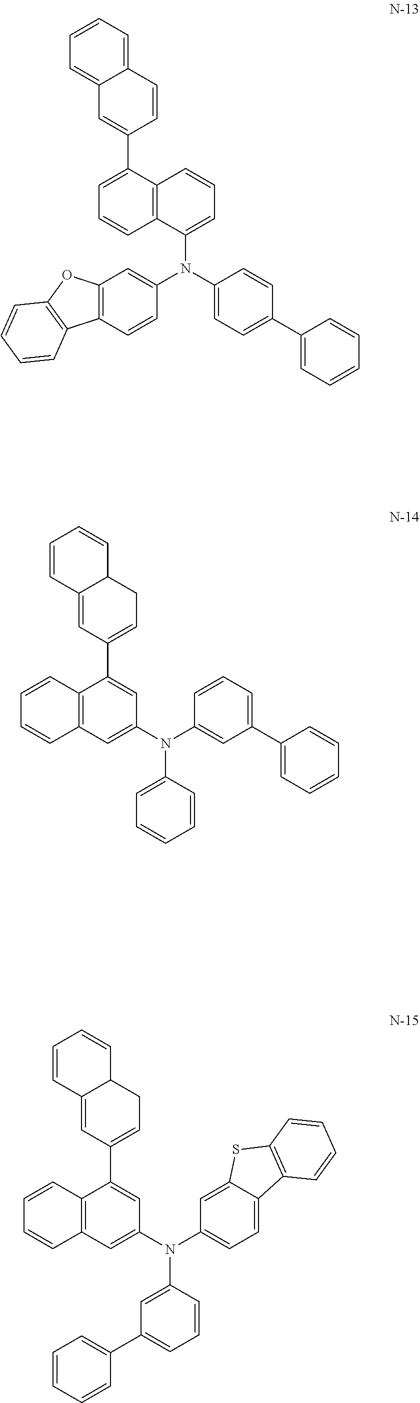 [SMILES: c1ccc(-c2ccc(N(c3ccc4c(c3)oc3ccccc34)c3cccc4c(-c5ccc6ccccc6c5)cccc34)cc2)cc1 ; C1=CC2=CC(c3cc(N(c4ccccc4)c4cccc(-c5ccccc5)c4)cc4ccccc34)=CCC2C=C1 ; C1=CC2=CC(c3cc(N(c4cccc(-c5ccccc5)c4)c4ccc5c(c4)sc4ccccc45)cc4ccccc34)=CCC2C=C1]

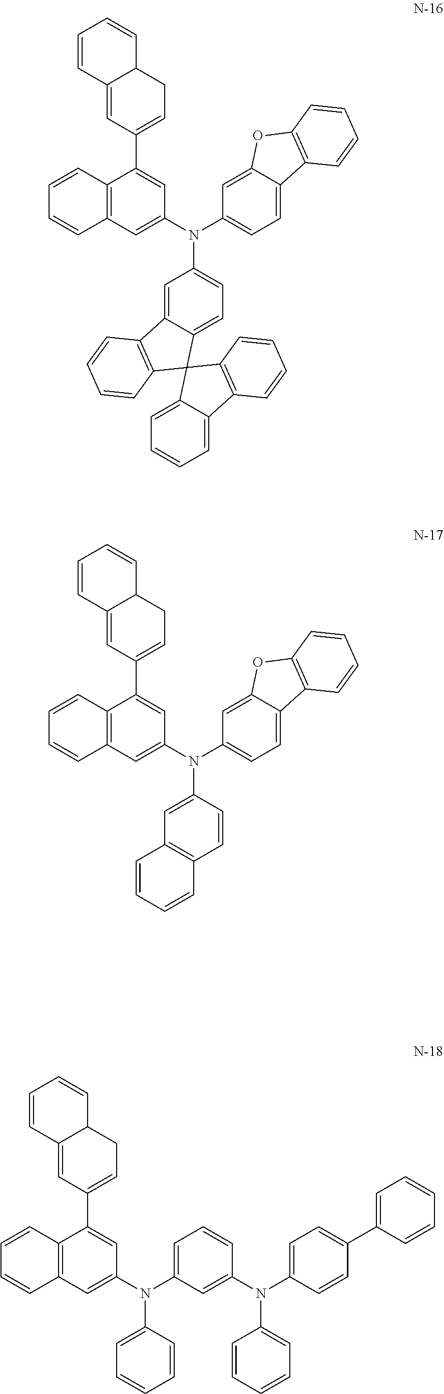 [SMILES: C1=CC2=CC(c3cc(N(c4ccc5c(c4)-c4ccccc4C54c5ccccc5-c5ccccc54)c4ccc5c(c4)oc4ccccc45)cc4ccccc34)=CCC2C=C1 ; C1=CC2=CC(c3cc(N(c4ccc5ccccc5c4)c4ccc5c(c4)oc4ccccc45)cc4ccccc34)=CCC2C=C1 ; C1=CC2=CC(c3cc(N(c4ccccc4)c4cccc(N(c5ccccc5)c5ccc(-c6ccccc6)cc5)c4)cc4ccccc34)=CCC2C=C1]

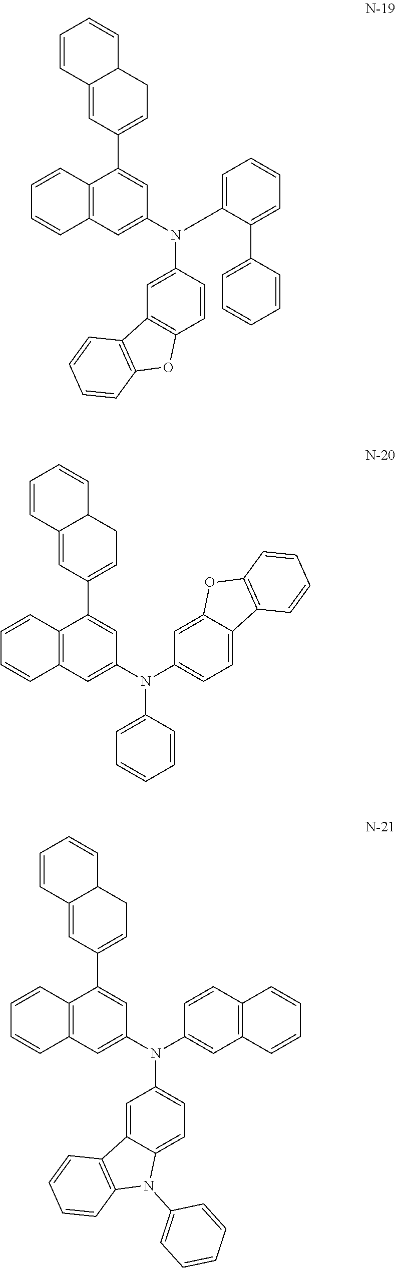 [SMILES: C1=CC2=CC(c3cc(N(c4ccc5oc6ccccc6c5c4)c4ccccc4-c4ccccc4)cc4ccccc34)=CCC2C=C1 ; C1=CC2=CC(c3cc(N(c4ccccc4)c4ccc5c(c4)oc4ccccc45)cc4ccccc34)=CCC2C=C1 ; C1=CC2=CC(c3cc(N(c4ccc5ccccc5c4)c4ccc5c(c4)c4ccccc4n5-c4ccccc4)cc4ccccc34)=CCC2C=C1]

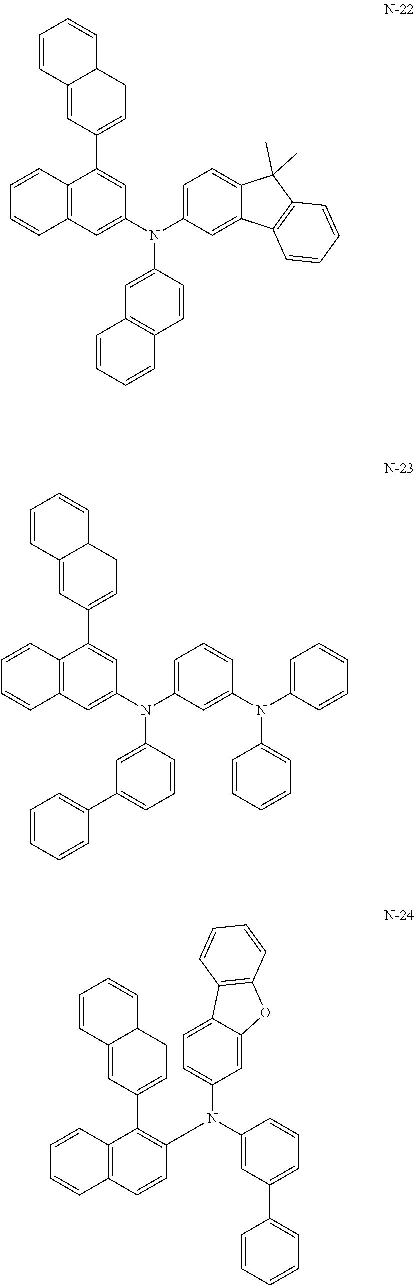 [SMILES: CC1(C)c2ccccc2-c2cc(N(c3ccc4ccccc4c3)c3cc(C4=CCC5C=CC=CC5=C4)c4ccccc4c3)ccc21 ; C1=CC2=CC(c3cc(N(c4cccc(-c5ccccc5)c4)c4cccc(N(c5ccccc5)c5ccccc5)c4)cc4ccccc34)=CCC2C=C1 ; C1=CC2=CC(c3c(N(c4cccc(-c5ccccc5)c4)c4ccc5c(c4)oc4ccccc45)ccc4ccccc34)=CCC2C=C1]

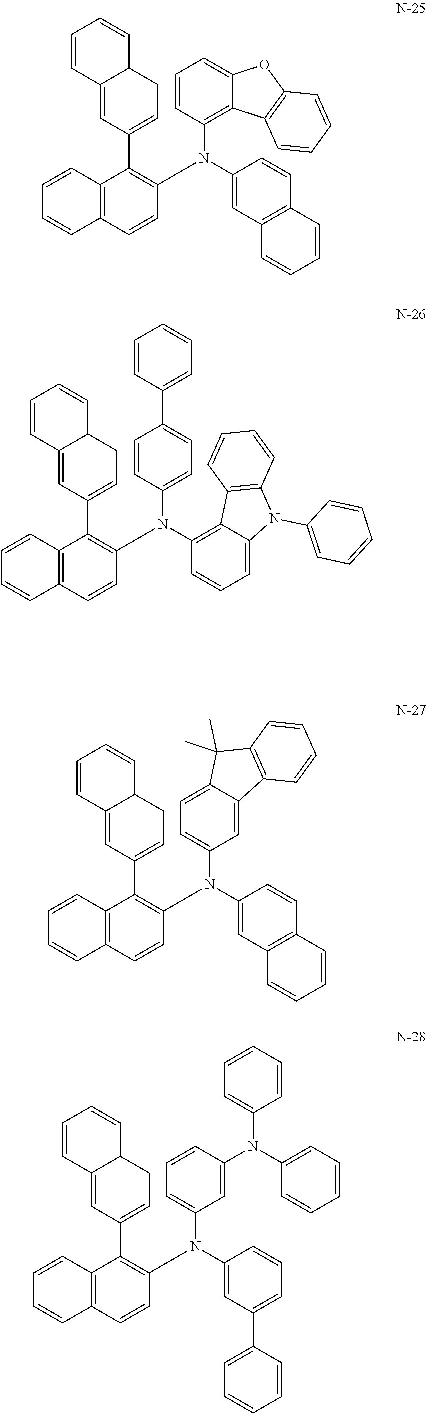 [SMILES: C1=CC2=CC(c3c(N(c4ccc5ccccc5c4)c4cccc5oc6ccccc6c45)ccc4ccccc34)=CCC2C=C1 ; C1=CC2=CC(c3c(N(c4ccc(-c5ccccc5)cc4)c4cccc5c4c4ccccc4n5-c4ccccc4)ccc4ccccc34)=CCC2C=C1 ; CC1(C)c2ccccc2-c2cc(N(c3ccc4ccccc4c3)c3ccc4ccccc4c3C3=CCC4C=CC=CC4=C3)ccc21 ; C1=CC2=CC(c3c(N(c4cccc(-c5ccccc5)c4)c4cccc(N(c5ccccc5)c5ccccc5)c4)ccc4ccccc34)=CCC2C=C1]

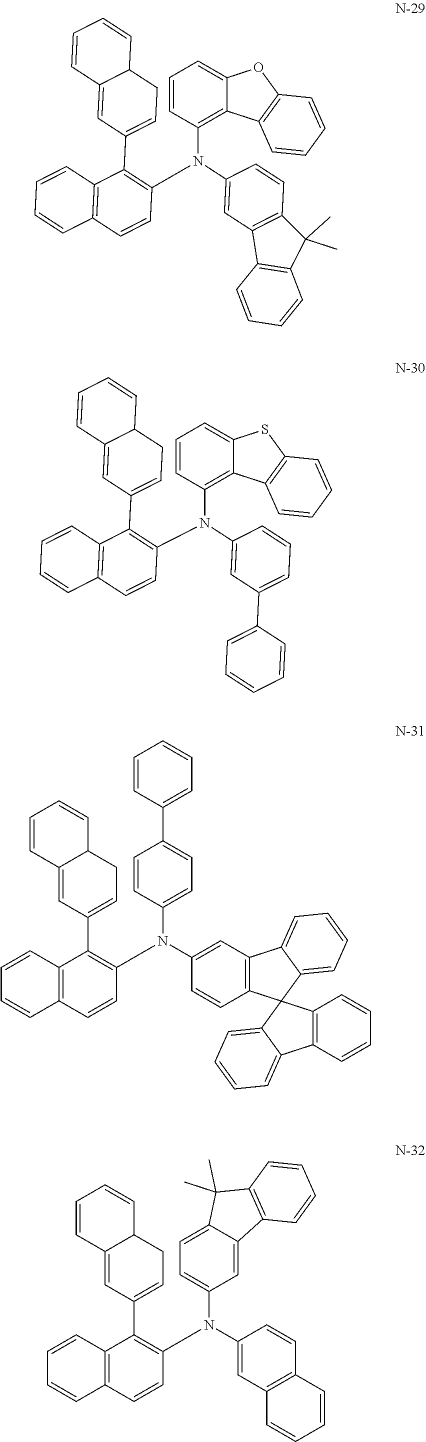 [SMILES: CC1(C)c2ccccc2-c2cc(N(c3ccc4ccccc4c3C3=CCC4C=CC=CC4=C3)c3cccc4oc5ccccc5c34)ccc21 ; C1=CC2=CC(c3c(N(c4cccc(-c5ccccc5)c4)c4cccc5sc6ccccc6c45)ccc4ccccc34)=CCC2C=C1 ; C1=CC2=CC(c3c(N(c4ccc(-c5ccccc5)cc4)c4ccc5c(c4)-c4ccccc4C54c5ccccc5-c5ccccc54)ccc4ccccc34)=CCC2C=C1 ; CC1(C)c2ccccc2-c2cc(N(c3ccc4ccccc4c3)c3ccc4ccccc4c3C3=CCC4C=CC=CC4=C3)ccc21]

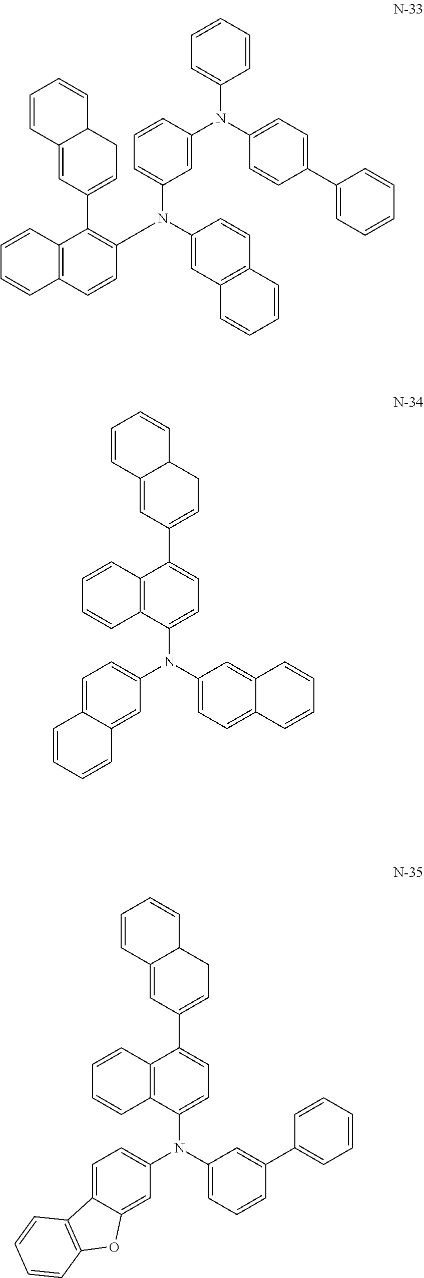 [SMILES: C1=CC2=CC(c3c(N(c4cccc(N(c5ccccc5)c5ccc(-c6ccccc6)cc5)c4)c4ccc5ccccc5c4)ccc4ccccc34)=CCC2C=C1 ; C1=CC2=CC(c3ccc(N(c4ccc5ccccc5c4)c4ccc5ccccc5c4)c4ccccc34)=CCC2C=C1 ; C1=CC2=CC(c3ccc(N(c4cccc(-c5ccccc5)c4)c4ccc5c(c4)oc4ccccc45)c4ccccc34)=CCC2C=C1]

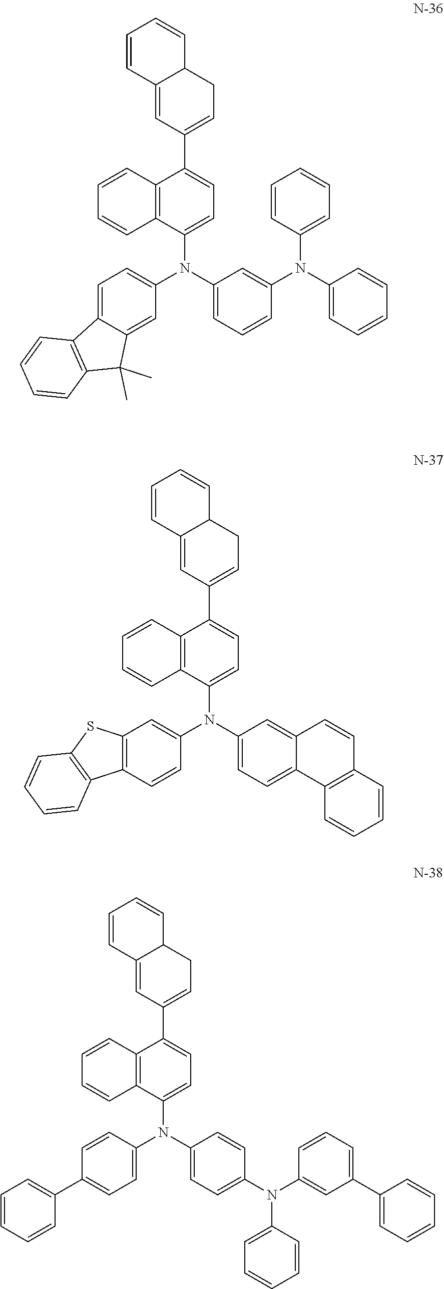 [SMILES: CC1(C)c2ccccc2-c2ccc(N(c3cccc(N(c4ccccc4)c4ccccc4)c3)c3ccc(C4=CCC5C=CC=CC5=C4)c4ccccc34)cc21 ; C1=CC2=CC(c3ccc(N(c4ccc5c(ccc6ccccc65)c4)c4ccc5c(c4)sc4ccccc45)c4ccccc34)=CCC2C=C1 ; C1=CC2=CC(c3ccc(N(c4ccc(-c5ccccc5)cc4)c4ccc(N(c5ccccc5)c5cccc(-c6ccccc6)c5)cc4)c4ccccc34)=CCC2C=C1]

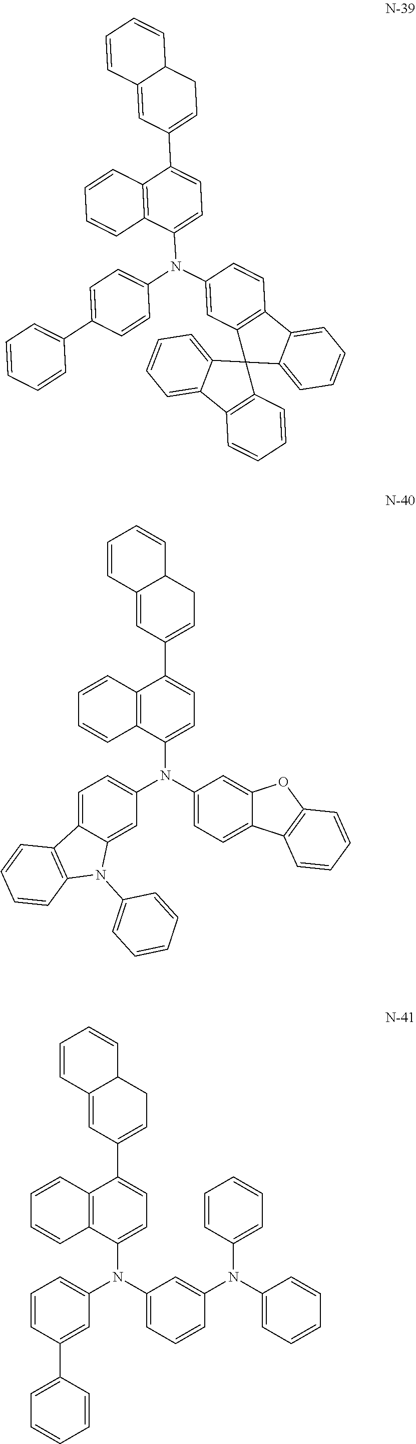 [SMILES: C1=CC2=CC(c3ccc(N(c4ccc(-c5ccccc5)cc4)c4ccc5c(c4)C4(c6ccccc6-c6ccccc64)c4ccccc4-5)c4ccccc34)=CCC2C=C1 ; C1=CC2=CC(c3ccc(N(c4ccc5c(c4)oc4ccccc45)c4ccc5c6ccccc6n(-c6ccccc6)c5c4)c4ccccc34)=CCC2C=C1 ; C1=CC2=CC(c3ccc(N(c4cccc(-c5ccccc5)c4)c4cccc(N(c5ccccc5)c5ccccc5)c4)c4ccccc34)=CCC2C=C1]

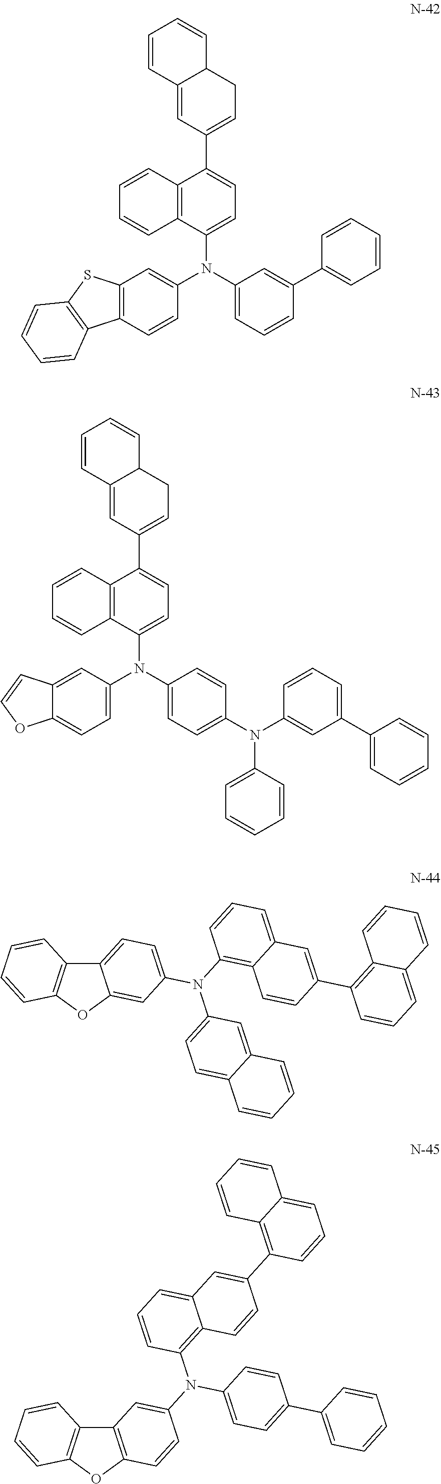 [SMILES: C1=CC2=CC(c3ccc(N(c4cccc(-c5ccccc5)c4)c4ccc5c(c4)sc4ccccc45)c4ccccc34)=CCC2C=C1 ; C1=CC2=CC(c3ccc(N(c4ccc(N(c5ccccc5)c5cccc(-c6ccccc6)c5)cc4)c4ccc5occc5c4)c4ccccc34)=CCC2C=C1 ; c1ccc2cc(N(c3ccc4c(c3)oc3ccccc34)c3cccc4cc(-c5cccc6ccccc56)ccc34)ccc2c1 ; c1ccc(-c2ccc(N(c3ccc4oc5ccccc5c4c3)c3cccc4cc(-c5cccc6ccccc56)ccc34)cc2)cc1]

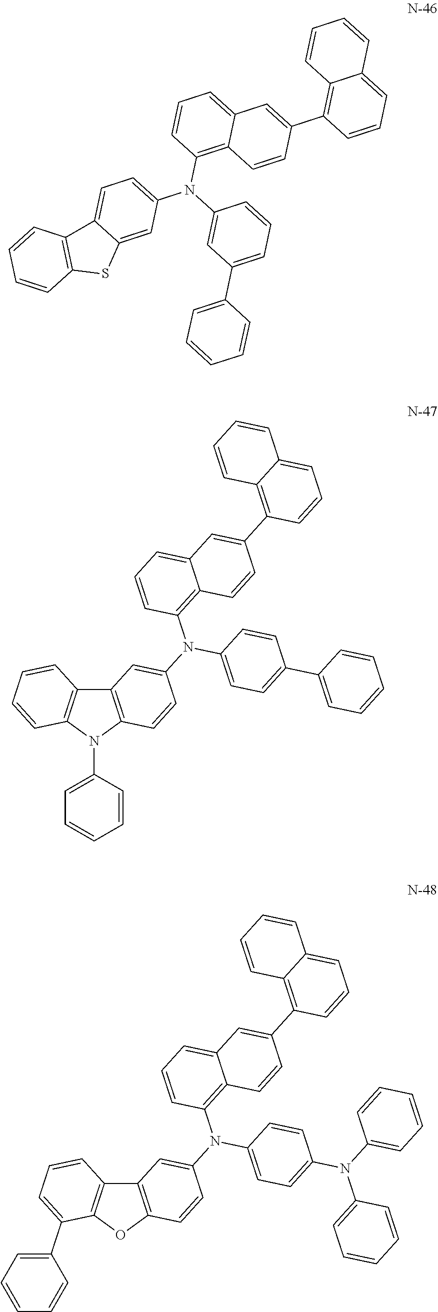 [SMILES: c1ccc(-c2cccc(N(c3ccc4c(c3)sc3ccccc34)c3cccc4cc(-c5cccc6ccccc56)ccc34)c2)cc1 ; c1ccc(-c2ccc(N(c3ccc4c(c3)c3ccccc3n4-c3ccccc3)c3cccc4cc(-c5cccc6ccccc56)ccc34)cc2)cc1 ; c1ccc(-c2cccc3c2oc2ccc(N(c4ccc(N(c5ccccc5)c5ccccc5)cc4)c4cccc5cc(-c6cccc7ccccc67)ccc45)cc23)cc1]

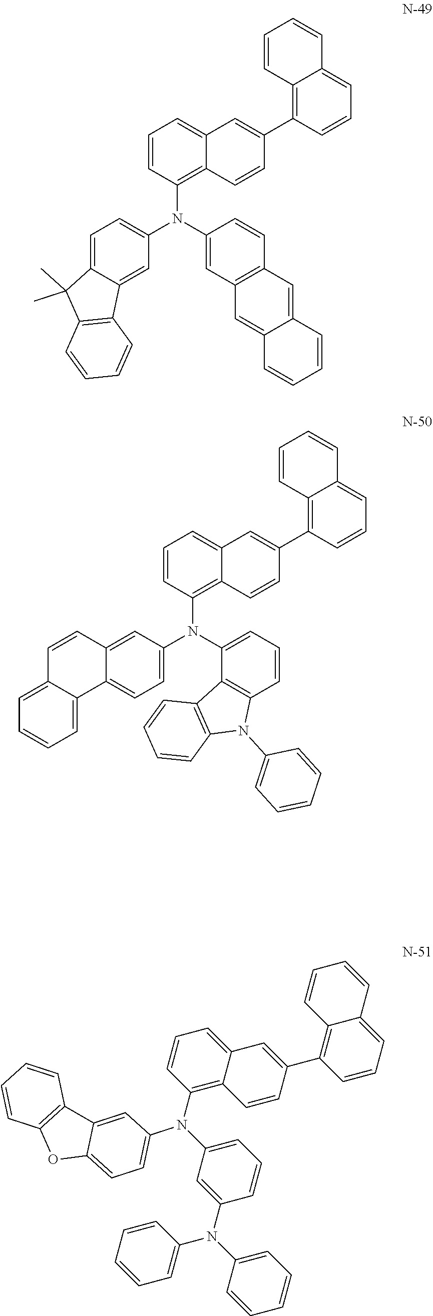 [SMILES: CC1(C)c2ccccc2-c2cc(N(c3ccc4cc5ccccc5cc4c3)c3cccc4cc(-c5cccc6ccccc56)ccc34)ccc21 ; c1ccc(-n2c3ccccc3c3c(N(c4ccc5c(ccc6ccccc65)c4)c4cccc5cc(-c6cccc7ccccc67)ccc45)cccc32)cc1 ; c1ccc(N(c2ccccc2)c2cccc(N(c3ccc4oc5ccccc5c4c3)c3cccc4cc(-c5cccc6ccccc56)ccc34)c2)cc1]

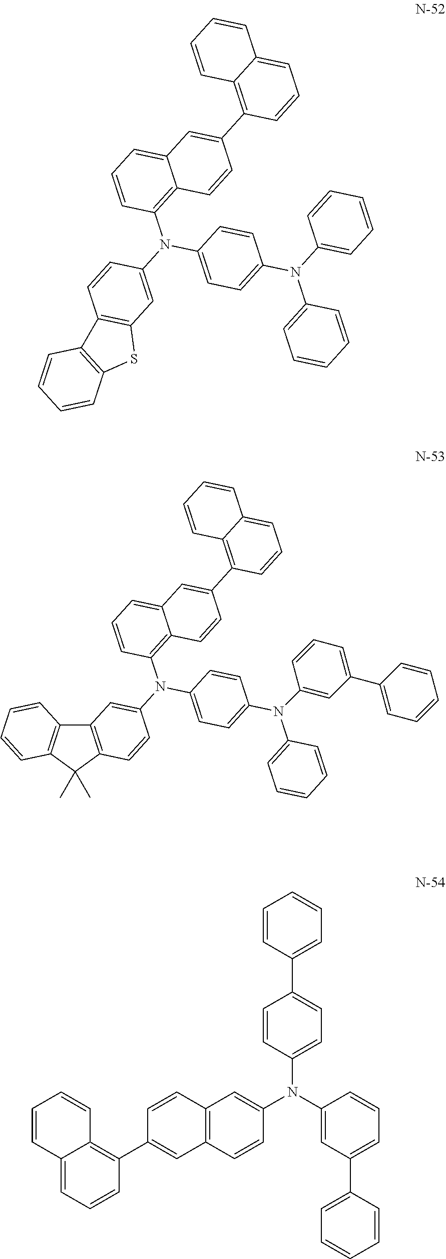 [SMILES: c1ccc(N(c2ccccc2)c2ccc(N(c3ccc4c(c3)sc3ccccc34)c3cccc4cc(-c5cccc6ccccc56)ccc34)cc2)cc1 ; CC1(C)c2ccccc2-c2cc(N(c3ccc(N(c4ccccc4)c4cccc(-c5ccccc5)c4)cc3)c3cccc4cc(-c5cccc6ccccc56)ccc34)ccc21 ; c1ccc(-c2ccc(N(c3cccc(-c4ccccc4)c3)c3ccc4cc(-c5cccc6ccccc56)ccc4c3)cc2)cc1]

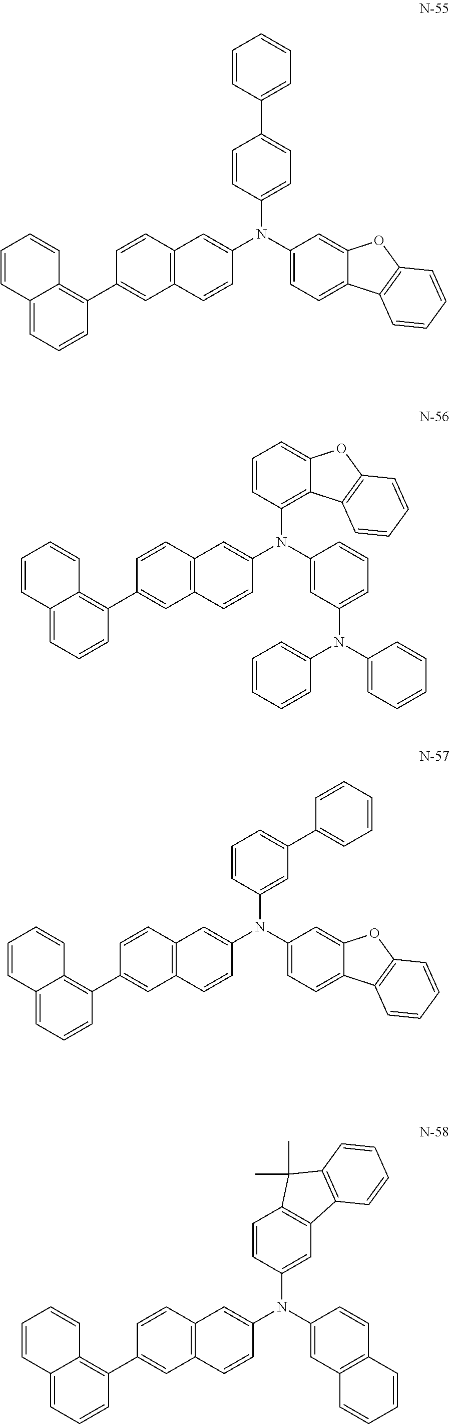 [SMILES: c1ccc(-c2ccc(N(c3ccc4cc(-c5cccc6ccccc56)ccc4c3)c3ccc4c(c3)oc3ccccc34)cc2)cc1 ; c1ccc(N(c2ccccc2)c2cccc(N(c3ccc4cc(-c5cccc6ccccc56)ccc4c3)c3cccc4oc5ccccc5c34)c2)cc1 ; c1ccc(-c2cccc(N(c3ccc4cc(-c5cccc6ccccc56)ccc4c3)c3ccc4c(c3)oc3ccccc34)c2)cc1 ; CC1(C)c2ccccc2-c2cc(N(c3ccc4ccccc4c3)c3ccc4cc(-c5cccc6ccccc56)ccc4c3)ccc21]

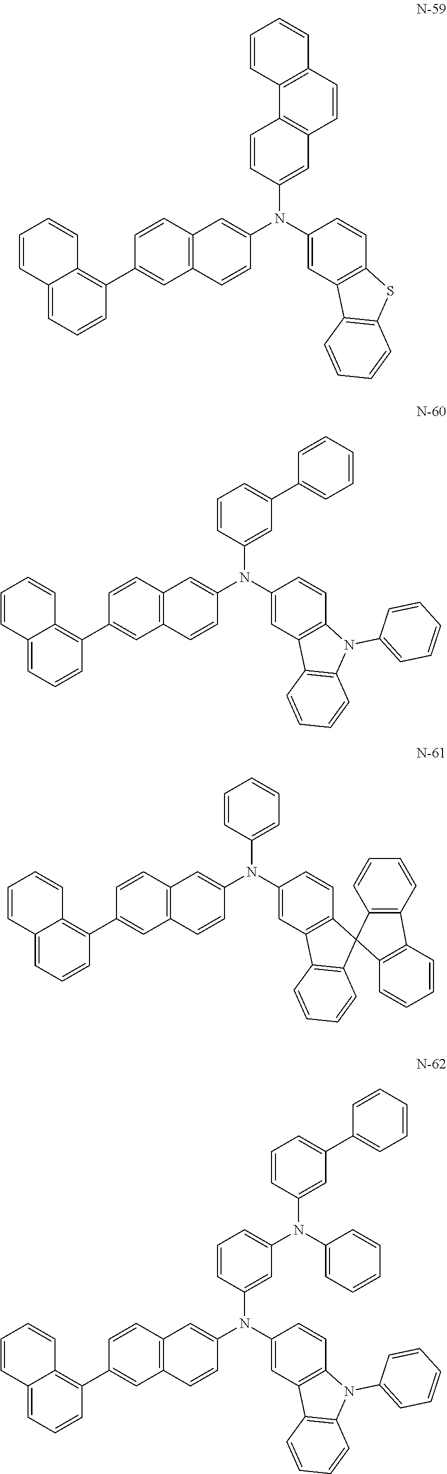 [SMILES: c1ccc2c(-c3ccc4cc(N(c5ccc6c(ccc7ccccc76)c5)c5ccc6sc7ccccc7c6c5)ccc4c3)cccc2c1 ; c1ccc(-c2cccc(N(c3ccc4cc(-c5cccc6ccccc56)ccc4c3)c3ccc4c(c3)c3ccccc3n4-c3ccccc3)c2)cc1 ; c1ccc(N(c2ccc3c(c2)-c2ccccc2C32c3ccccc3-c3ccccc32)c2ccc3cc(-c4cccc5ccccc45)ccc3c2)cc1 ; c1ccc(-c2cccc(N(c3ccccc3)c3cccc(N(c4ccc5cc(-c6cccc7ccccc67)ccc5c4)c4ccc5c(c4)c4ccccc4n5-c4ccccc4)c3)c2)cc1]

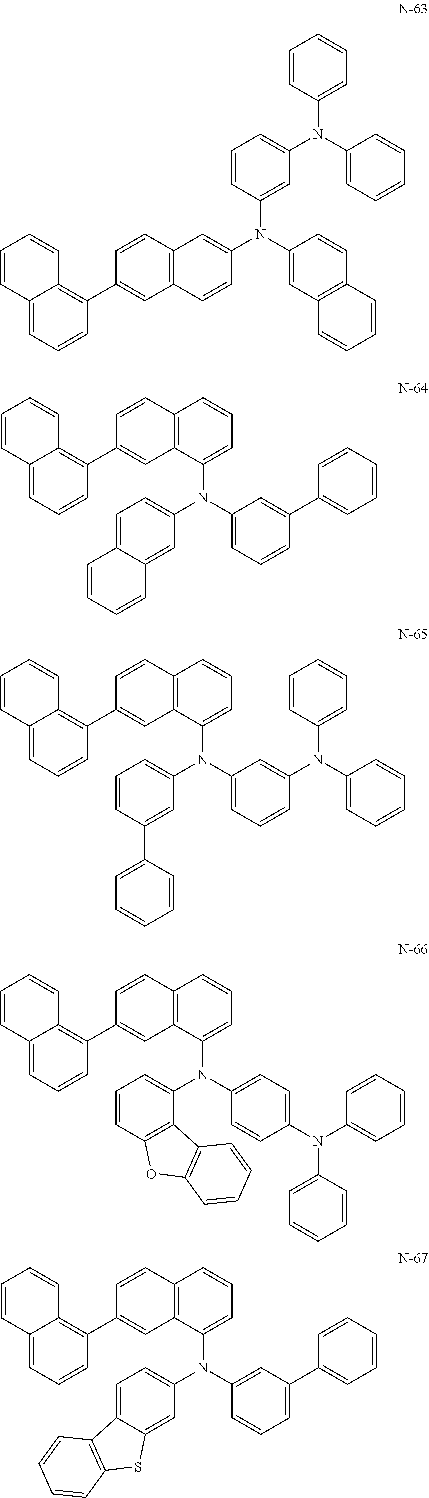 [SMILES: c1ccc(N(c2ccccc2)c2cccc(N(c3ccc4ccccc4c3)c3ccc4cc(-c5cccc6ccccc56)ccc4c3)c2)cc1 ; c1ccc(-c2cccc(N(c3ccc4ccccc4c3)c3cccc4ccc(-c5cccc6ccccc56)cc34)c2)cc1 ; c1ccc(-c2cccc(N(c3cccc(N(c4ccccc4)c4ccccc4)c3)c3cccc4ccc(-c5cccc6ccccc56)cc34)c2)cc1 ; c1ccc(N(c2ccccc2)c2ccc(N(c3cccc4ccc(-c5cccc6ccccc56)cc34)c3cccc4oc5ccccc5c34)cc2)cc1 ; c1ccc(-c2cccc(N(c3ccc4c(c3)sc3ccccc34)c3cccc4ccc(-c5cccc6ccccc56)cc34)c2)cc1]

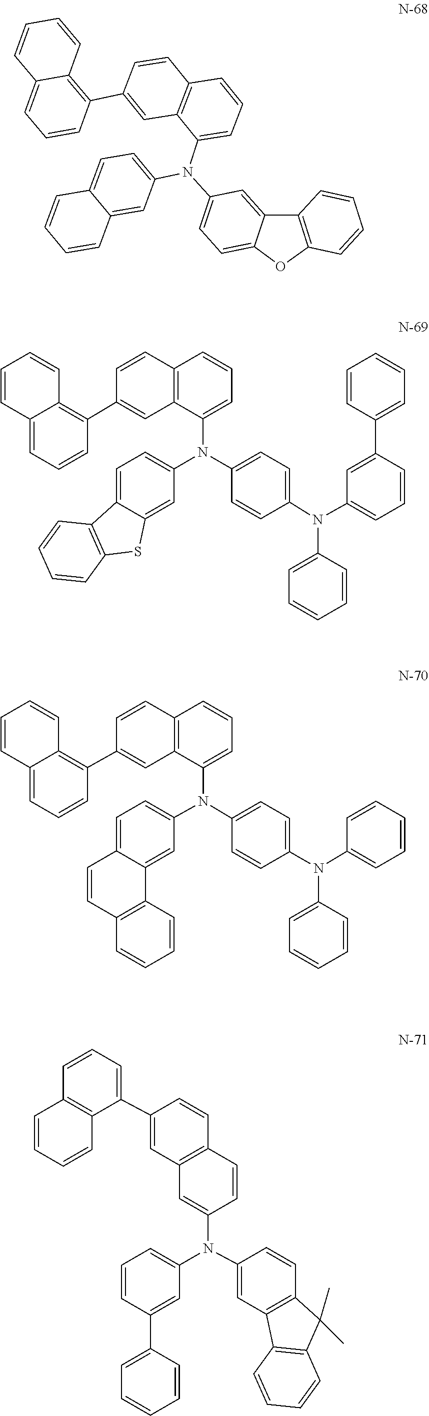 [SMILES: c1ccc2cc(N(c3ccc4oc5ccccc5c4c3)c3cccc4ccc(-c5cccc6ccccc56)cc34)ccc2c1 ; c1ccc(-c2cccc(N(c3ccccc3)c3ccc(N(c4ccc5c(c4)sc4ccccc45)c4cccc5ccc(-c6cccc7ccccc67)cc45)cc3)c2)cc1 ; c1ccc(N(c2ccccc2)c2ccc(N(c3ccc4ccc5ccccc5c4c3)c3cccc4ccc(-c5cccc6ccccc56)cc34)cc2)cc1 ; CC1(C)c2ccccc2-c2cc(N(c3cccc(-c4ccccc4)c3)c3ccc4ccc(-c5cccc6ccccc56)cc4c3)ccc21]

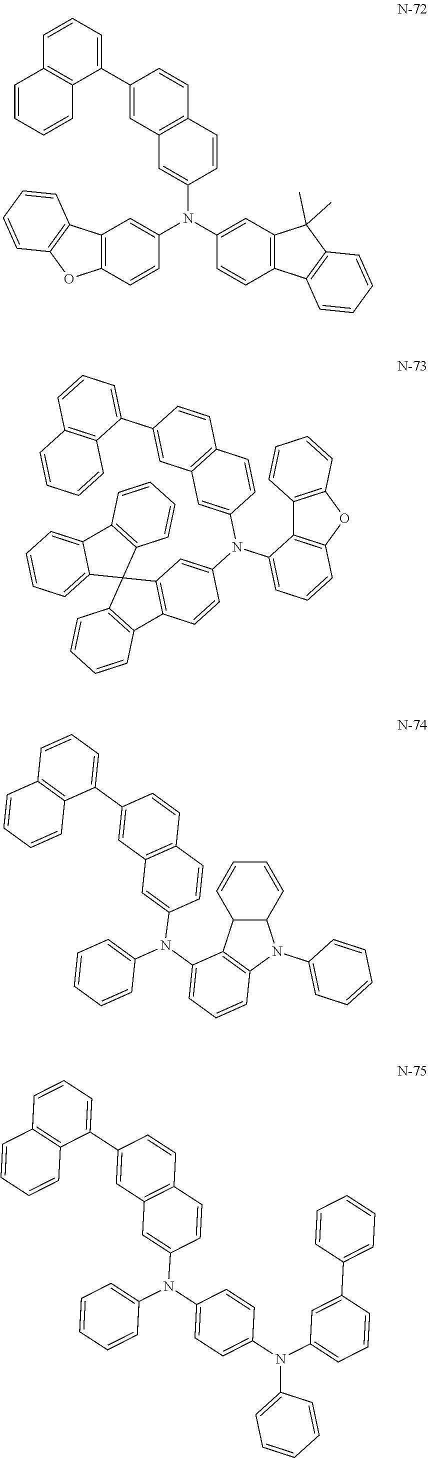 [SMILES: CC1(C)c2ccccc2-c2ccc(N(c3ccc4ccc(-c5cccc6ccccc56)cc4c3)c3ccc4oc5ccccc5c4c3)cc21 ; c1ccc2c(c1)-c1ccccc1C21c2ccccc2-c2ccc(N(c3ccc4ccc(-c5cccc6ccccc56)cc4c3)c3cccc4oc5ccccc5c34)cc21 ; C1=CC2c3c(N(c4ccccc4)c4ccc5ccc(-c6cccc7ccccc67)cc5c4)cccc3N(c3ccccc3)C2C=C1 ; c1ccc(-c2cccc(N(c3ccccc3)c3ccc(N(c4ccccc4)c4ccc5ccc(-c6cccc7ccccc67)cc5c4)cc3)c2)cc1]

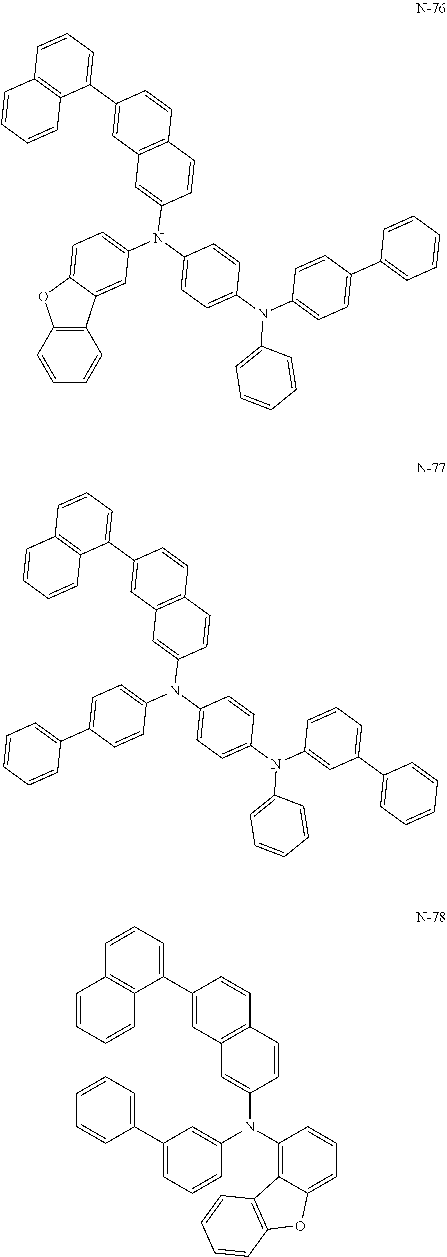 [SMILES: c1ccc(-c2ccc(N(c3ccccc3)c3ccc(N(c4ccc5ccc(-c6cccc7ccccc67)cc5c4)c4ccc5oc6ccccc6c5c4)cc3)cc2)cc1 ; c1ccc(-c2ccc(N(c3ccc(N(c4ccccc4)c4cccc(-c5ccccc5)c4)cc3)c3ccc4ccc(-c5cccc6ccccc56)cc4c3)cc2)cc1 ; c1ccc(-c2cccc(N(c3ccc4ccc(-c5cccc6ccccc56)cc4c3)c3cccc4oc5ccccc5c34)c2)cc1]

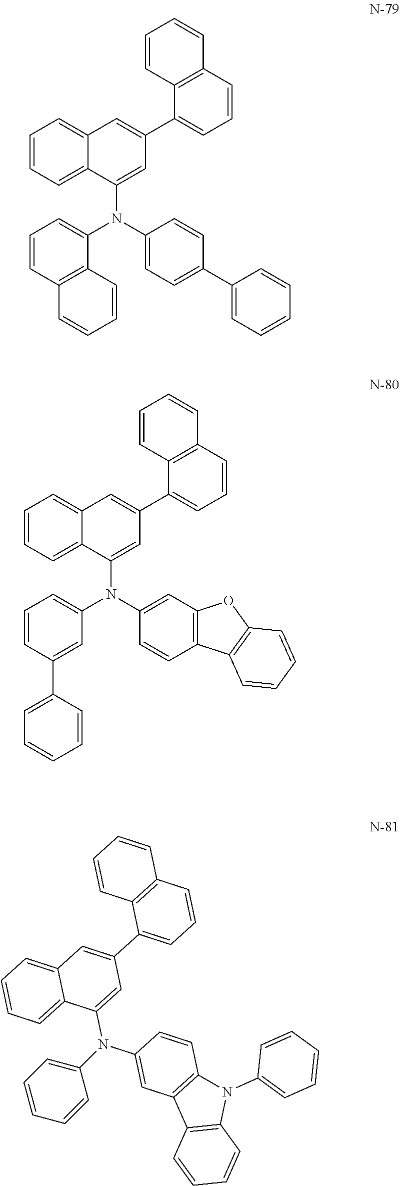 [SMILES: c1ccc(-c2ccc(N(c3cccc4ccccc34)c3cc(-c4cccc5ccccc45)cc4ccccc34)cc2)cc1 ; c1ccc(-c2cccc(N(c3ccc4c(c3)oc3ccccc34)c3cc(-c4cccc5ccccc45)cc4ccccc34)c2)cc1 ; c1ccc(N(c2ccc3c(c2)c2ccccc2n3-c2ccccc2)c2cc(-c3cccc4ccccc34)cc3ccccc23)cc1]

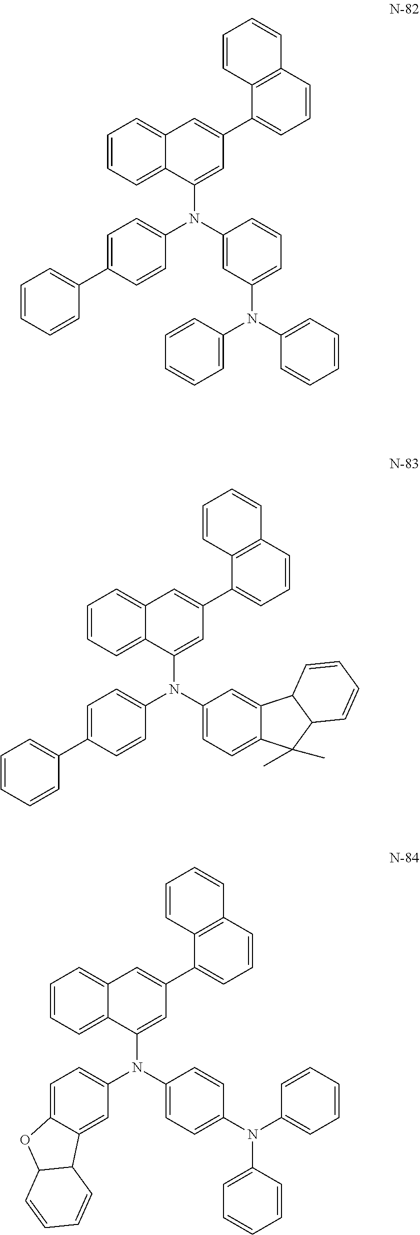 [SMILES: c1ccc(-c2ccc(N(c3cccc(N(c4ccccc4)c4ccccc4)c3)c3cc(-c4cccc5ccccc45)cc4ccccc34)cc2)cc1 ; CC1(C)c2ccc(N(c3ccc(-c4ccccc4)cc3)c3cc(-c4cccc5ccccc45)cc4ccccc34)cc2C2C=CC=CC21 ; C1=CC2Oc3ccc(N(c4ccc(N(c5ccccc5)c5ccccc5)cc4)c4cc(-c5cccc6ccccc56)cc5ccccc45)cc3C2C=C1]

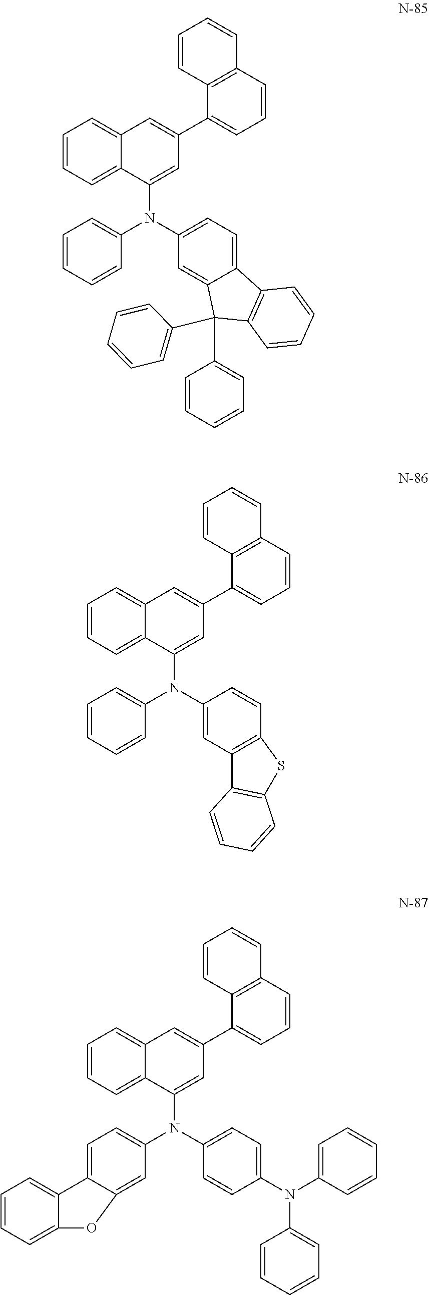 [SMILES: c1ccc(N(c2ccc3c(c2)C(c2ccccc2)(c2ccccc2)c2ccccc2-3)c2cc(-c3cccc4ccccc34)cc3ccccc23)cc1 ; c1ccc(N(c2ccc3sc4ccccc4c3c2)c2cc(-c3cccc4ccccc34)cc3ccccc23)cc1 ; c1ccc(N(c2ccccc2)c2ccc(N(c3ccc4c(c3)oc3ccccc34)c3cc(-c4cccc5ccccc45)cc4ccccc34)cc2)cc1]

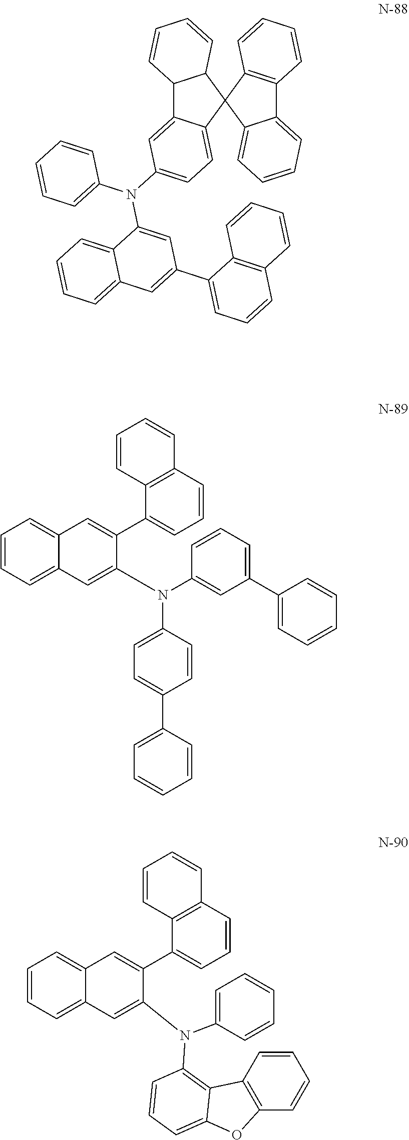 [SMILES: C1=CC2c3cc(N(c4ccccc4)c4cc(-c5cccc6ccccc56)cc5ccccc45)ccc3C3(c4ccccc4-c4ccccc43)C2C=C1 ; c1ccc(-c2ccc(N(c3cccc(-c4ccccc4)c3)c3cc4ccccc4cc3-c3cccc4ccccc34)cc2)cc1 ; c1ccc(N(c2cc3ccccc3cc2-c2cccc3ccccc23)c2cccc3oc4ccccc4c23)cc1]

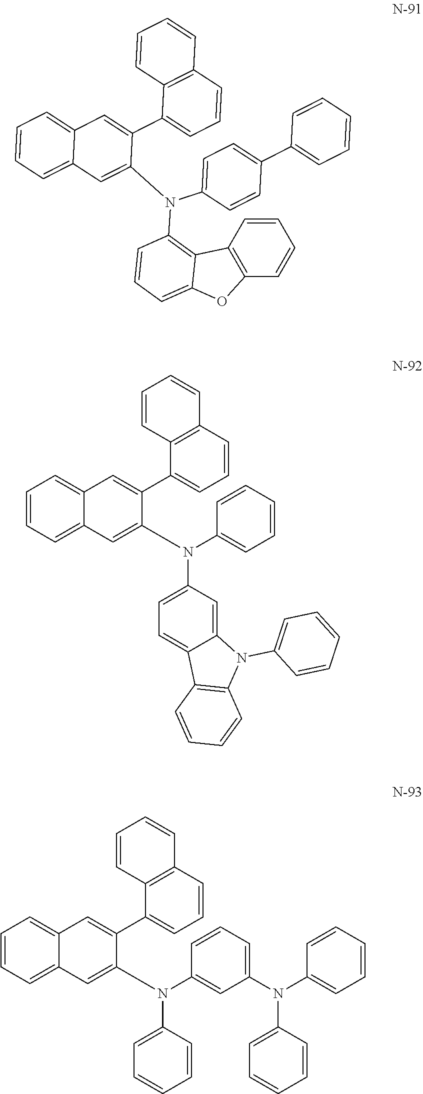 [SMILES: c1ccc(-c2ccc(N(c3cc4ccccc4cc3-c3cccc4ccccc34)c3cccc4oc5ccccc5c34)cc2)cc1 ; c1ccc(N(c2ccc3c4ccccc4n(-c4ccccc4)c3c2)c2cc3ccccc3cc2-c2cccc3ccccc23)cc1 ; c1ccc(N(c2ccccc2)c2cccc(N(c3ccccc3)c3cc4ccccc4cc3-c3cccc4ccccc34)c2)cc1]

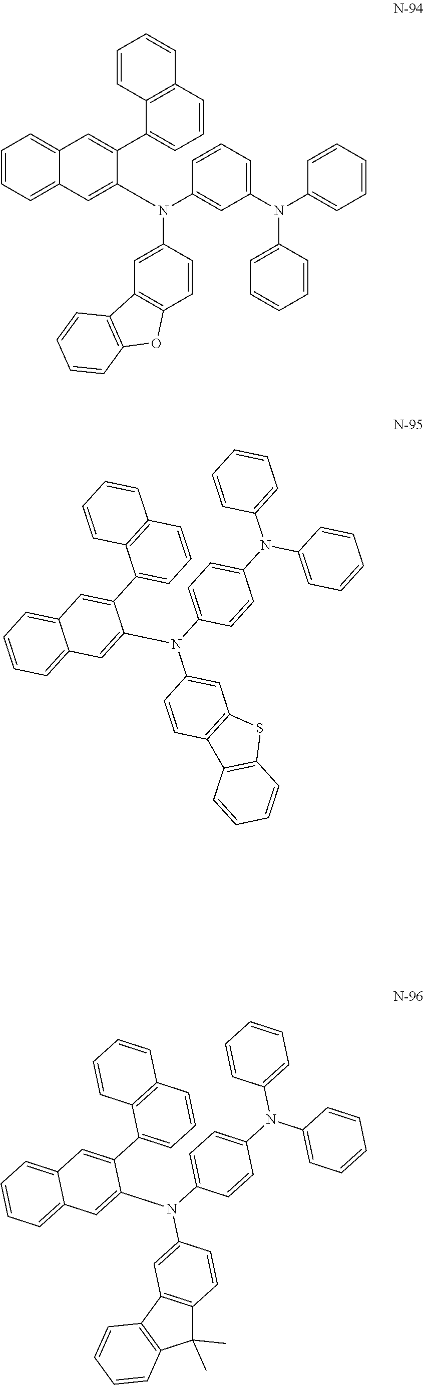 [SMILES: c1ccc(N(c2ccccc2)c2cccc(N(c3ccc4oc5ccccc5c4c3)c3cc4ccccc4cc3-c3cccc4ccccc34)c2)cc1 ; c1ccc(N(c2ccccc2)c2ccc(N(c3ccc4c(c3)sc3ccccc34)c3cc4ccccc4cc3-c3cccc4ccccc34)cc2)cc1 ; CC1(C)c2ccccc2-c2cc(N(c3ccc(N(c4ccccc4)c4ccccc4)cc3)c3cc4ccccc4cc3-c3cccc4ccccc34)ccc21]

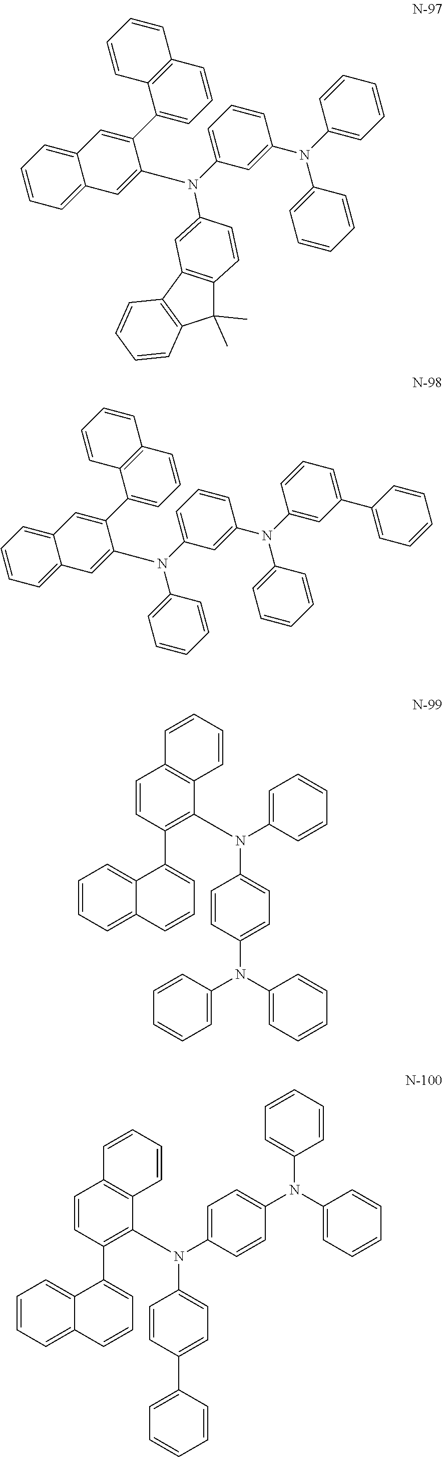 [SMILES: CC1(C)c2ccccc2-c2cc(N(c3cccc(N(c4ccccc4)c4ccccc4)c3)c3cc4ccccc4cc3-c3cccc4ccccc34)ccc21 ; c1ccc(-c2cccc(N(c3ccccc3)c3cccc(N(c4ccccc4)c4cc5ccccc5cc4-c4cccc5ccccc45)c3)c2)cc1 ; c1ccc(N(c2ccccc2)c2ccc(N(c3ccccc3)c3c(-c4cccc5ccccc45)ccc4ccccc34)cc2)cc1 ; c1ccc(-c2ccc(N(c3ccc(N(c4ccccc4)c4ccccc4)cc3)c3c(-c4cccc5ccccc45)ccc4ccccc34)cc2)cc1]

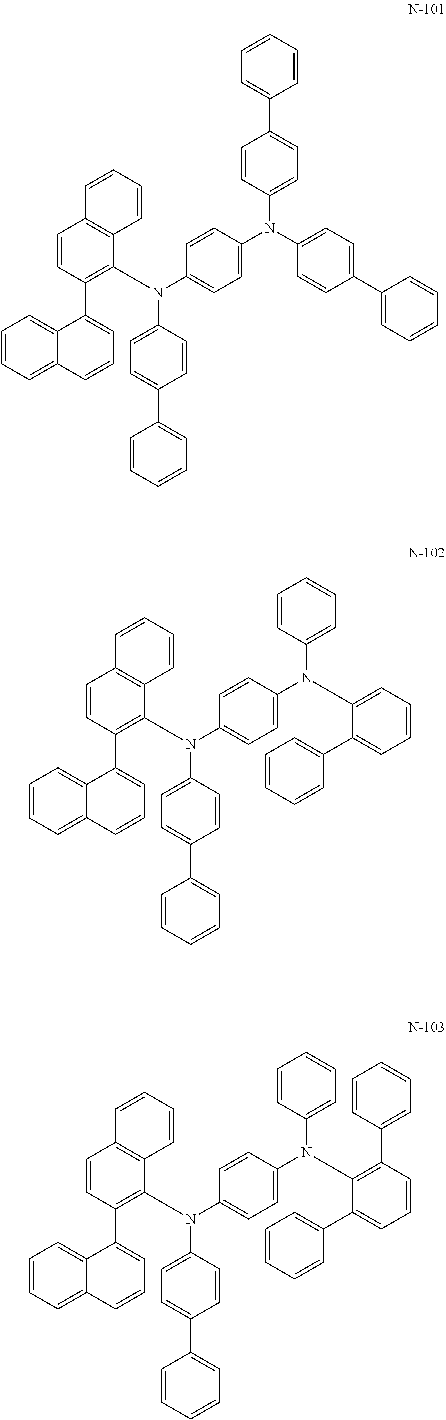 [SMILES: c1ccc(-c2ccc(N(c3ccc(-c4ccccc4)cc3)c3ccc(N(c4ccc(-c5ccccc5)cc4)c4c(-c5cccc6ccccc56)ccc5ccccc45)cc3)cc2)cc1 ; c1ccc(-c2ccc(N(c3ccc(N(c4ccccc4)c4ccccc4-c4ccccc4)cc3)c3c(-c4cccc5ccccc45)ccc4ccccc34)cc2)cc1 ; c1ccc(-c2ccc(N(c3ccc(N(c4ccccc4)c4c(-c5ccccc5)cccc4-c4ccccc4)cc3)c3c(-c4cccc5ccccc45)ccc4ccccc34)cc2)cc1]

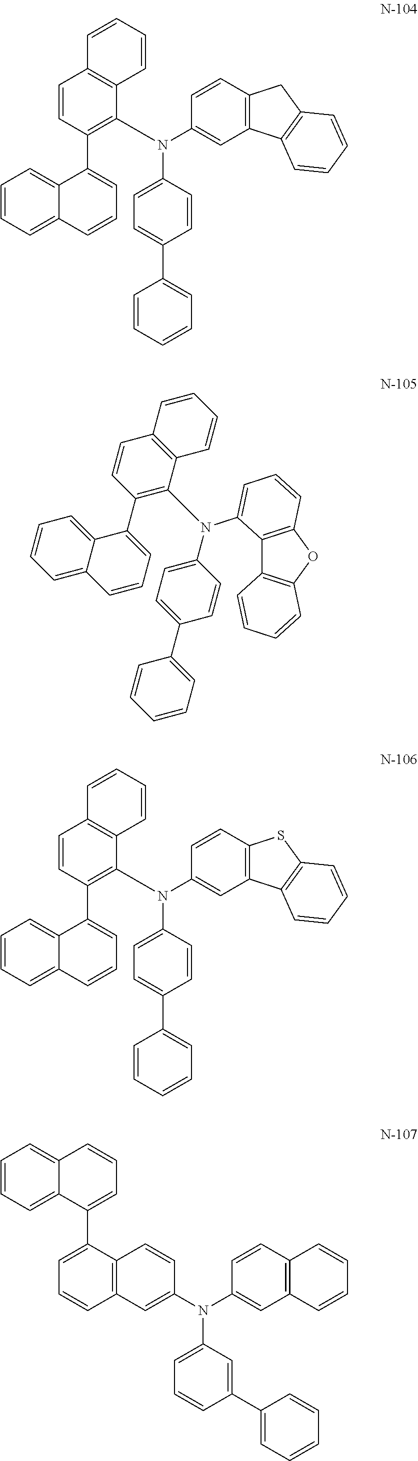 [SMILES: c1ccc(-c2ccc(N(c3ccc4c(c3)-c3ccccc3C4)c3c(-c4cccc5ccccc45)ccc4ccccc34)cc2)cc1 ; c1ccc(-c2ccc(N(c3c(-c4cccc5ccccc45)ccc4ccccc34)c3cccc4oc5ccccc5c34)cc2)cc1 ; c1ccc(-c2ccc(N(c3ccc4sc5ccccc5c4c3)c3c(-c4cccc5ccccc45)ccc4ccccc34)cc2)cc1 ; c1ccc(-c2cccc(N(c3ccc4ccccc4c3)c3ccc4c(-c5cccc6ccccc56)cccc4c3)c2)cc1]

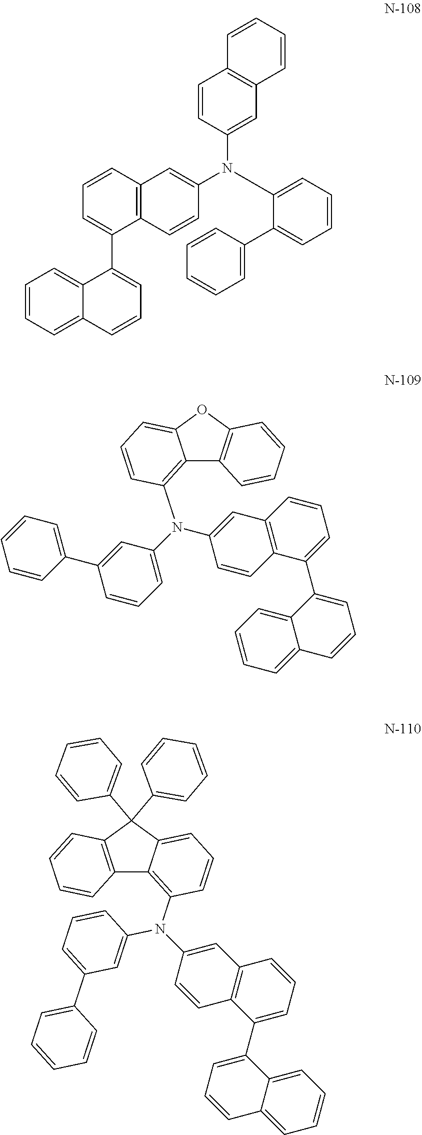 [SMILES: c1ccc(-c2ccccc2N(c2ccc3ccccc3c2)c2ccc3c(-c4cccc5ccccc45)cccc3c2)cc1 ; c1ccc(-c2cccc(N(c3ccc4c(-c5cccc6ccccc56)cccc4c3)c3cccc4oc5ccccc5c34)c2)cc1 ; c1ccc(-c2cccc(N(c3ccc4c(-c5cccc6ccccc56)cccc4c3)c3cccc4c3-c3ccccc3C4(c3ccccc3)c3ccccc3)c2)cc1]

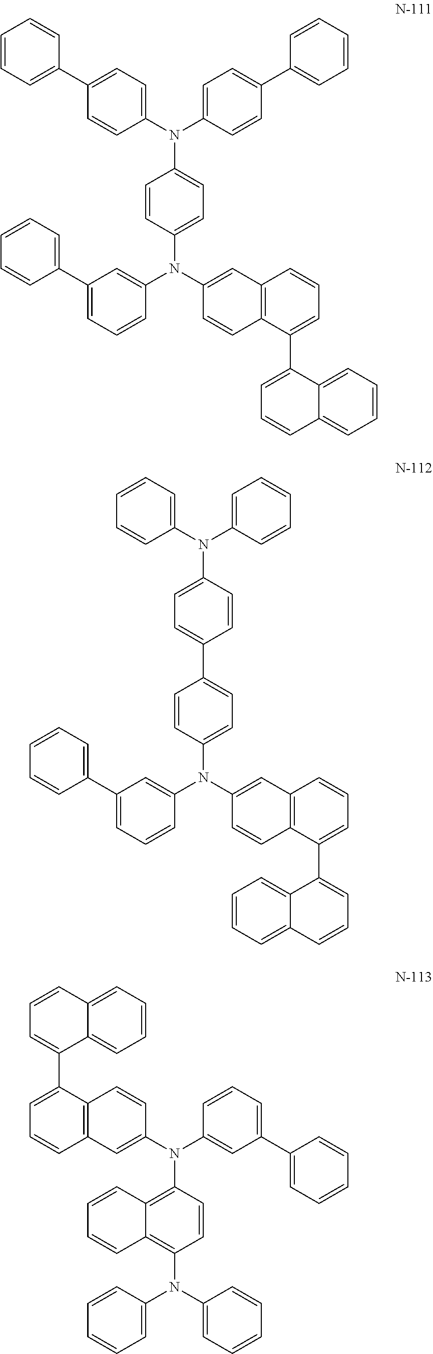 [SMILES: c1ccc(-c2ccc(N(c3ccc(-c4ccccc4)cc3)c3ccc(N(c4cccc(-c5ccccc5)c4)c4ccc5c(-c6cccc7ccccc67)cccc5c4)cc3)cc2)cc1 ; c1ccc(-c2cccc(N(c3ccc(-c4ccc(N(c5ccccc5)c5ccccc5)cc4)cc3)c3ccc4c(-c5cccc6ccccc56)cccc4c3)c2)cc1 ; c1ccc(-c2cccc(N(c3ccc4c(-c5cccc6ccccc56)cccc4c3)c3ccc(N(c4ccccc4)c4ccccc4)c4ccccc34)c2)cc1]

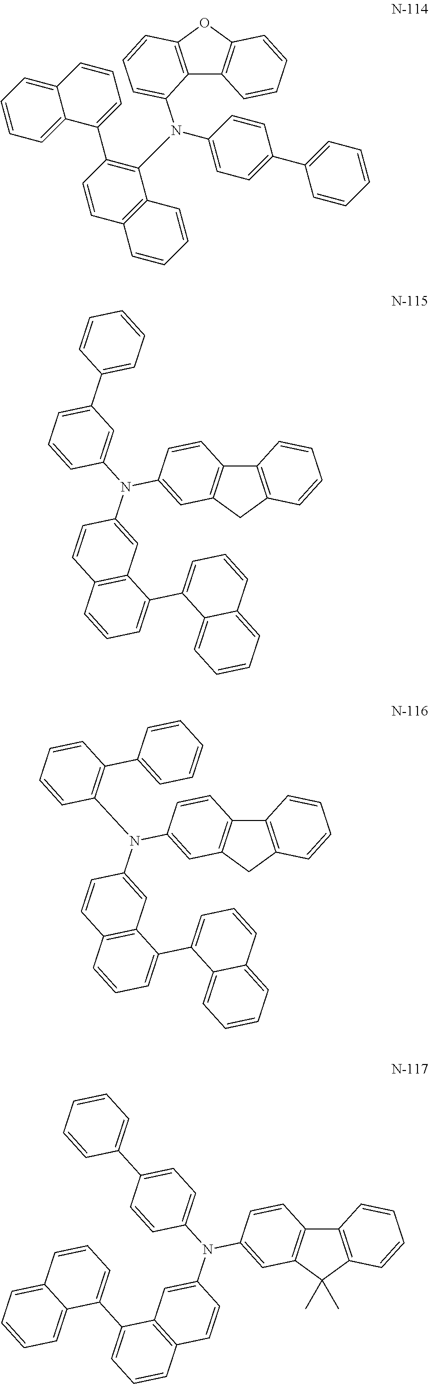 [SMILES: c1ccc(-c2ccc(N(c3c(-c4cccc5ccccc45)ccc4ccccc34)c3cccc4oc5ccccc5c34)cc2)cc1 ; c1ccc(-c2cccc(N(c3ccc4c(c3)Cc3ccccc3-4)c3ccc4cccc(-c5cccc6ccccc56)c4c3)c2)cc1 ; c1ccc(-c2ccccc2N(c2ccc3c(c2)Cc2ccccc2-3)c2ccc3cccc(-c4cccc5ccccc45)c3c2)cc1 ; CC1(C)c2ccccc2-c2ccc(N(c3ccc(-c4ccccc4)cc3)c3ccc4cccc(-c5cccc6ccccc56)c4c3)cc21]

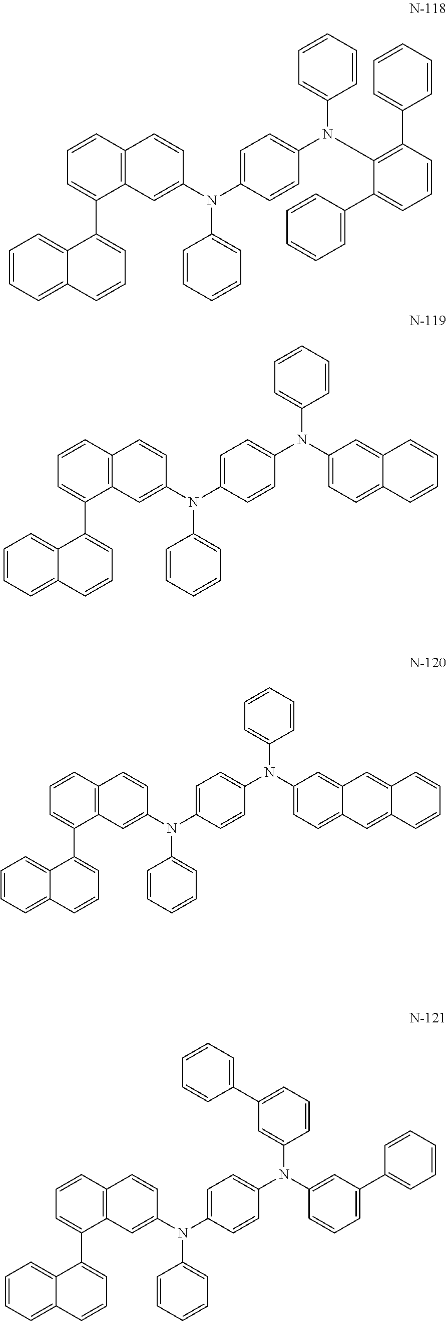 [SMILES: c1ccc(-c2cccc(-c3ccccc3)c2N(c2ccccc2)c2ccc(N(c3ccccc3)c3ccc4cccc(-c5cccc6ccccc56)c4c3)cc2)cc1 ; c1ccc(N(c2ccc(N(c3ccccc3)c3ccc4cccc(-c5cccc6ccccc56)c4c3)cc2)c2ccc3ccccc3c2)cc1 ; c1ccc(N(c2ccc(N(c3ccccc3)c3ccc4cccc(-c5cccc6ccccc56)c4c3)cc2)c2ccc3cc4ccccc4cc3c2)cc1 ; c1ccc(-c2cccc(N(c3ccc(N(c4ccccc4)c4ccc5cccc(-c6cccc7ccccc67)c5c4)cc3)c3cccc(-c4ccccc4)c3)c2)cc1]

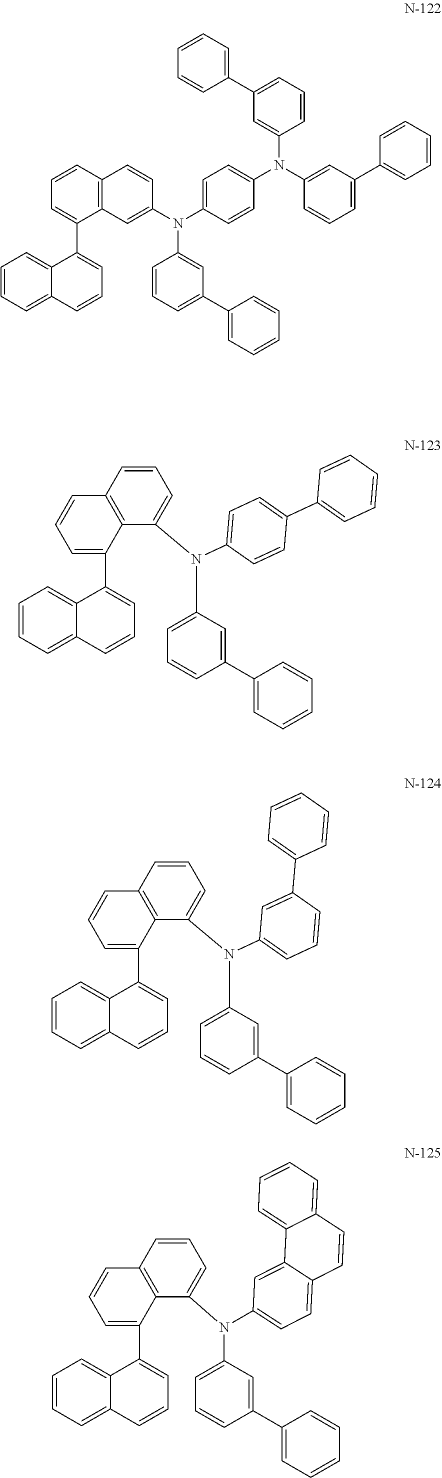

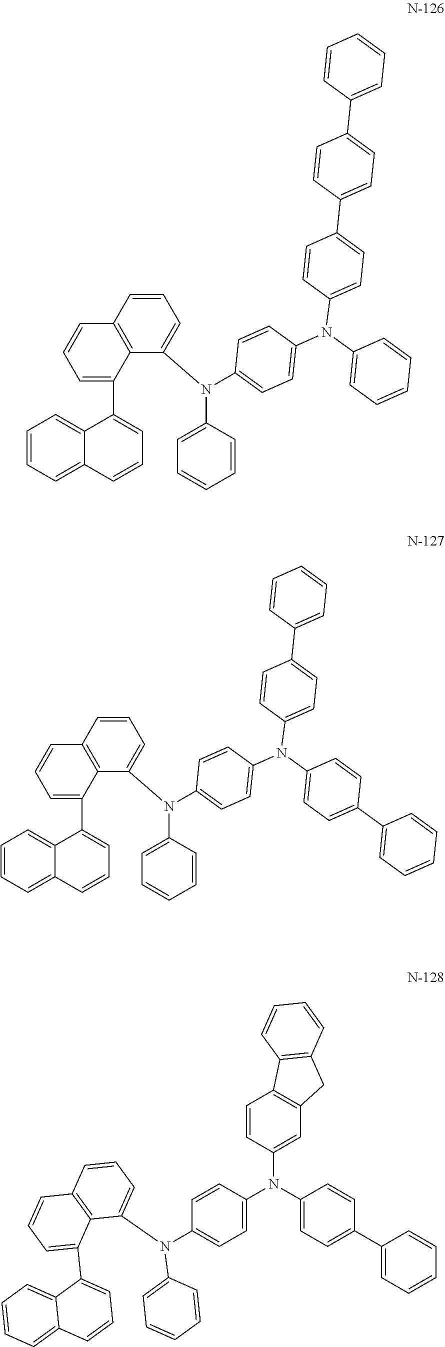 [SMILES: c1ccc(-c2ccc(-c3ccc(N(c4ccccc4)c4ccc(N(c5ccccc5)c5cccc6cccc(-c7cccc8ccccc78)c56)cc4)cc3)cc2)cc1 ; c1ccc(-c2ccc(N(c3ccc(-c4ccccc4)cc3)c3ccc(N(c4ccccc4)c4cccc5cccc(-c6cccc7ccccc67)c45)cc3)cc2)cc1 ; c1ccc(-c2ccc(N(c3ccc(N(c4ccccc4)c4cccc5cccc(-c6cccc7ccccc67)c45)cc3)c3ccc4c(c3)Cc3ccccc3-4)cc2)cc1]

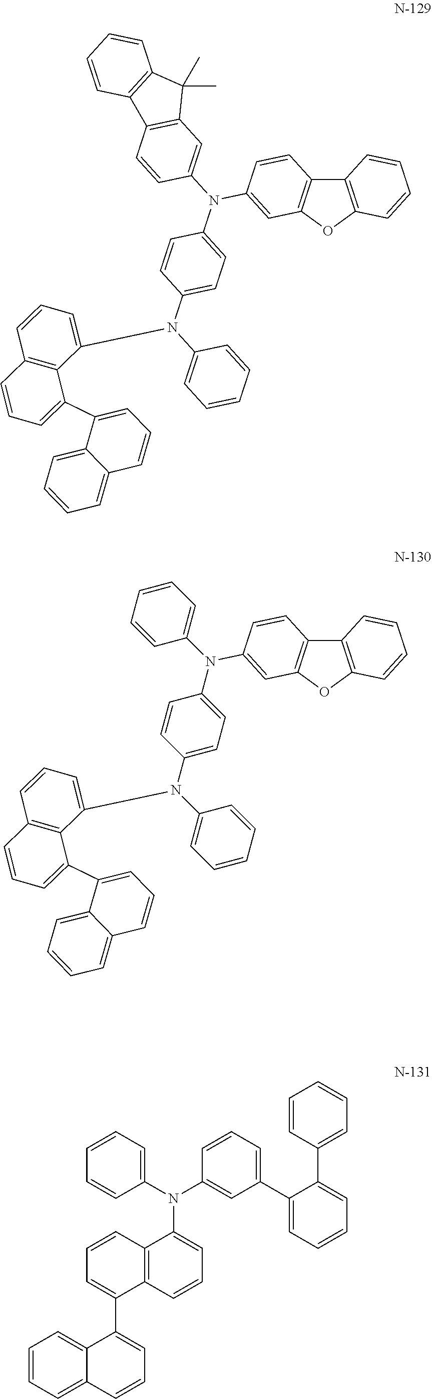 [SMILES: CC1(C)c2ccccc2-c2ccc(N(c3ccc(N(c4ccccc4)c4cccc5cccc(-c6cccc7ccccc67)c45)cc3)c3ccc4c(c3)oc3ccccc34)cc21 ; c1ccc(N(c2ccc(N(c3ccccc3)c3cccc4cccc(-c5cccc6ccccc56)c34)cc2)c2ccc3c(c2)oc2ccccc23)cc1 ; c1ccc(-c2ccccc2-c2cccc(N(c3ccccc3)c3cccc4c(-c5cccc6ccccc56)cccc34)c2)cc1]

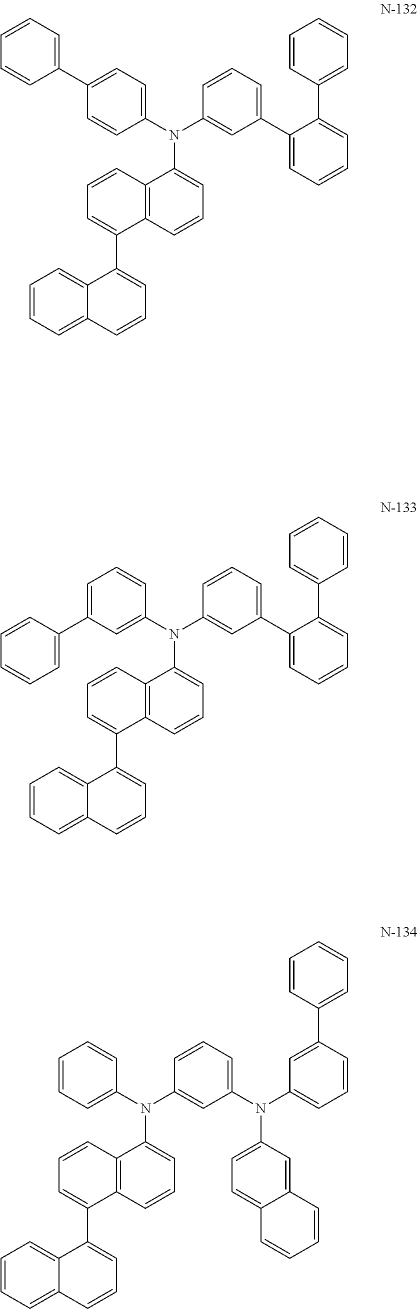 [SMILES: c1ccc(-c2ccc(N(c3cccc(-c4ccccc4-c4ccccc4)c3)c3cccc4c(-c5cccc6ccccc56)cccc34)cc2)cc1 ; c1ccc(-c2cccc(N(c3cccc(-c4ccccc4-c4ccccc4)c3)c3cccc4c(-c5cccc6ccccc56)cccc34)c2)cc1 ; c1ccc(-c2cccc(N(c3cccc(N(c4ccccc4)c4cccc5c(-c6cccc7ccccc67)cccc45)c3)c3ccc4ccccc4c3)c2)cc1]

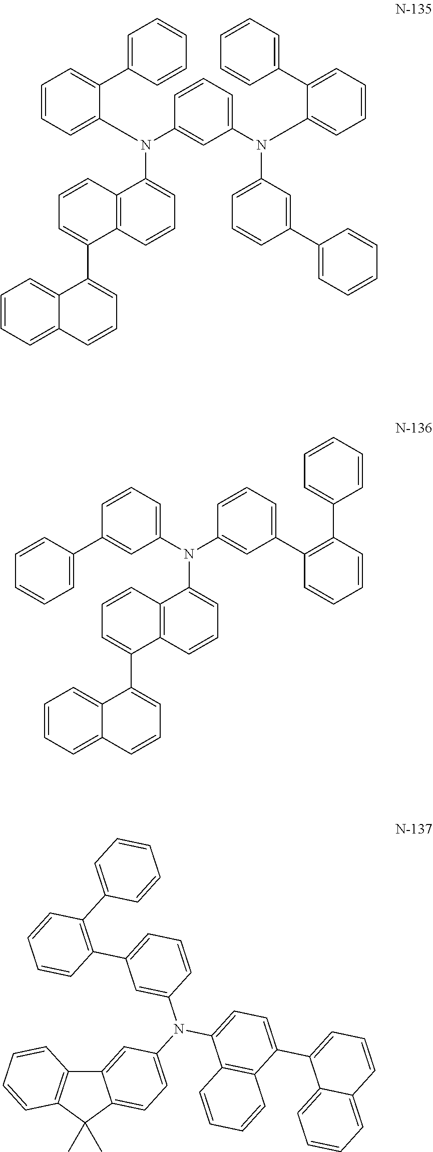 [SMILES: c1ccc(-c2cccc(N(c3cccc(N(c4ccccc4-c4ccccc4)c4cccc5c(-c6cccc7ccccc67)cccc45)c3)c3ccccc3-c3ccccc3)c2)cc1 ; c1ccc(-c2cccc(N(c3cccc(-c4ccccc4-c4ccccc4)c3)c3cccc4c(-c5cccc6ccccc56)cccc34)c2)cc1 ; CC1(C)c2ccccc2-c2cc(N(c3cccc(-c4ccccc4-c4ccccc4)c3)c3ccc(-c4cccc5ccccc45)c4ccccc34)ccc21]

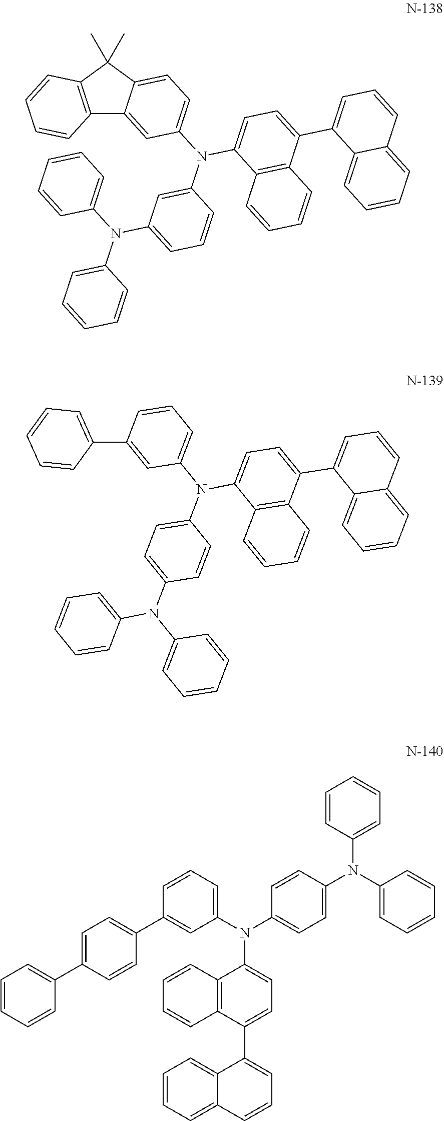 [SMILES: CC1(C)c2ccccc2-c2cc(N(c3cccc(N(c4ccccc4)c4ccccc4)c3)c3ccc(-c4cccc5ccccc45)c4ccccc34)ccc21 ; c1ccc(-c2cccc(N(c3ccc(N(c4ccccc4)c4ccccc4)cc3)c3ccc(-c4cccc5ccccc45)c4ccccc34)c2)cc1 ; c1ccc(-c2ccc(-c3cccc(N(c4ccc(N(c5ccccc5)c5ccccc5)cc4)c4ccc(-c5cccc6ccccc56)c5ccccc45)c3)cc2)cc1]

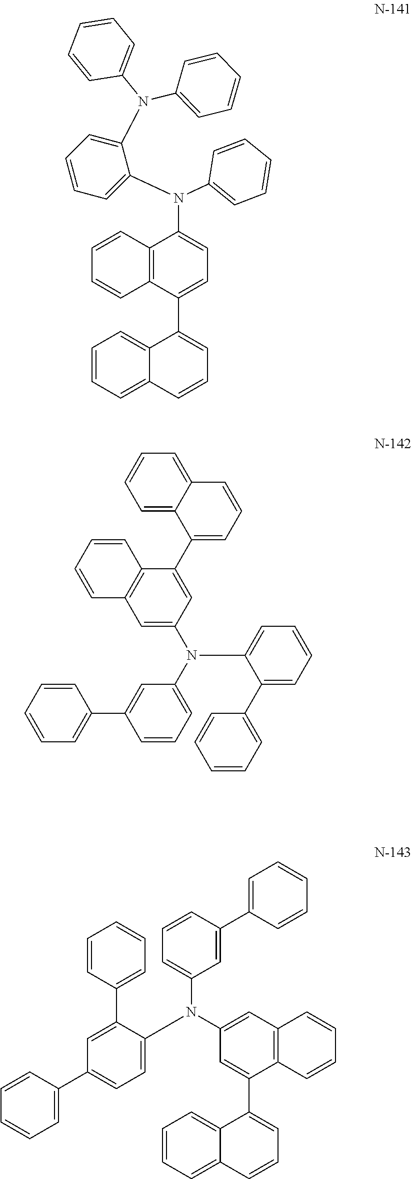 [SMILES: c1ccc(N(c2ccccc2)c2ccccc2N(c2ccccc2)c2ccc(-c3cccc4ccccc34)c3ccccc23)cc1 ; c1ccc(-c2cccc(N(c3cc(-c4cccc5ccccc45)c4ccccc4c3)c3ccccc3-c3ccccc3)c2)cc1 ; c1ccc(-c2cccc(N(c3cc(-c4cccc5ccccc45)c4ccccc4c3)c3ccc(-c4ccccc4)cc3-c3ccccc3)c2)cc1]

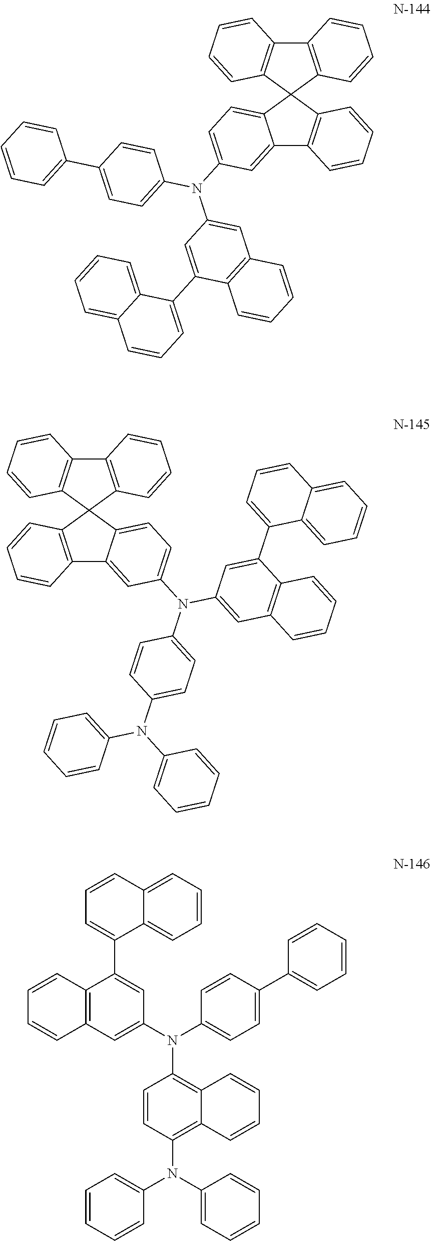 [SMILES: c1ccc(-c2ccc(N(c3ccc4c(c3)-c3ccccc3C43c4ccccc4-c4ccccc43)c3cc(-c4cccc5ccccc45)c4ccccc4c3)cc2)cc1 ; c1ccc(N(c2ccccc2)c2ccc(N(c3ccc4c(c3)-c3ccccc3C43c4ccccc4-c4ccccc43)c3cc(-c4cccc5ccccc45)c4ccccc4c3)cc2)cc1 ; c1ccc(-c2ccc(N(c3cc(-c4cccc5ccccc45)c4ccccc4c3)c3ccc(N(c4ccccc4)c4ccccc4)c4ccccc34)cc2)cc1]

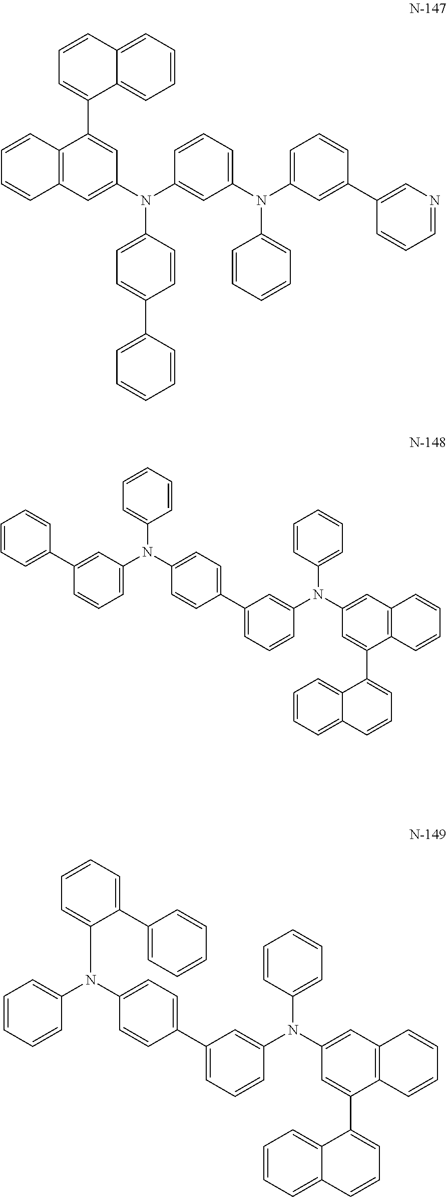 [SMILES: c1ccc(-c2ccc(N(c3cccc(N(c4ccccc4)c4cccc(-c5cccnc5)c4)c3)c3cc(-c4cccc5ccccc45)c4ccccc4c3)cc2)cc1 ; c1ccc(-c2cccc(N(c3ccccc3)c3ccc(-c4cccc(N(c5ccccc5)c5cc(-c6cccc7ccccc67)c6ccccc6c5)c4)cc3)c2)cc1 ; c1ccc(-c2ccccc2N(c2ccccc2)c2ccc(-c3cccc(N(c4ccccc4)c4cc(-c5cccc6ccccc56)c5ccccc5c4)c3)cc2)cc1]

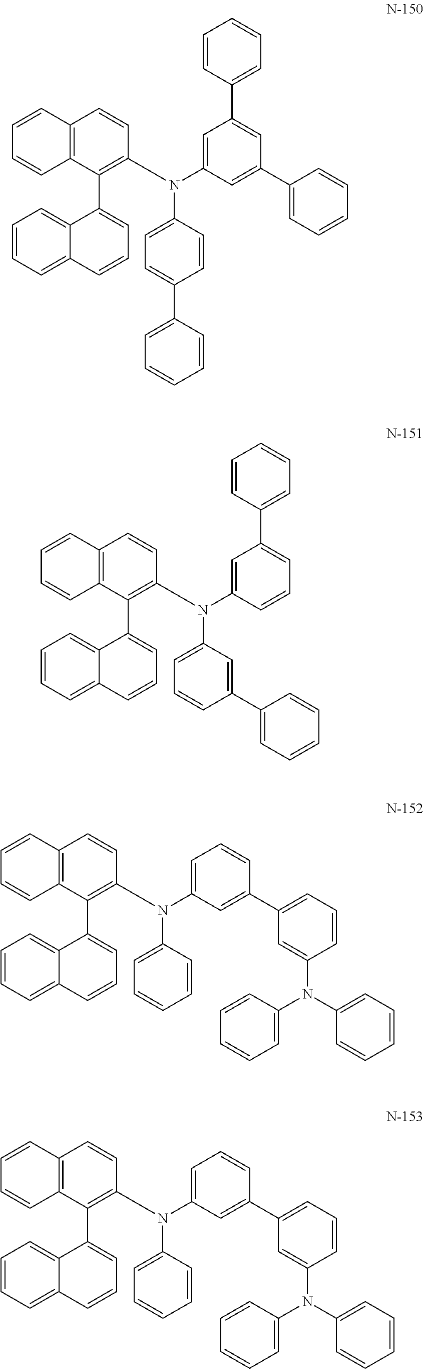 [SMILES: c1ccc(-c2ccc(N(c3cc(-c4ccccc4)cc(-c4ccccc4)c3)c3ccc4ccccc4c3-c3cccc4ccccc34)cc2)cc1 ; c1ccc(-c2cccc(N(c3cccc(-c4ccccc4)c3)c3ccc4ccccc4c3-c3cccc4ccccc34)c2)cc1 ; c1ccc(N(c2ccccc2)c2cccc(-c3cccc(N(c4ccccc4)c4ccc5ccccc5c4-c4cccc5ccccc45)c3)c2)cc1 ; c1ccc(N(c2ccccc2)c2cccc(-c3cccc(N(c4ccccc4)c4ccc5ccccc5c4-c4cccc5ccccc45)c3)c2)cc1]

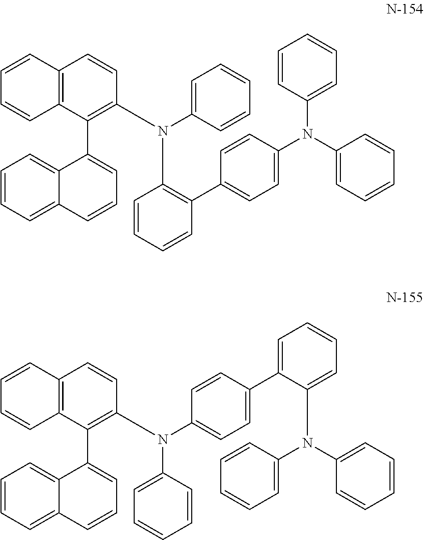 [SMILES: c1ccc(N(c2ccccc2)c2ccc(-c3ccccc3N(c3ccccc3)c3ccc4ccccc4c3-c3cccc4ccccc34)cc2)cc1 ; c1ccc(N(c2ccccc2)c2ccccc2-c2ccc(N(c3ccccc3)c3ccc4ccccc4c3-c3cccc4ccccc34)cc2)cc1]

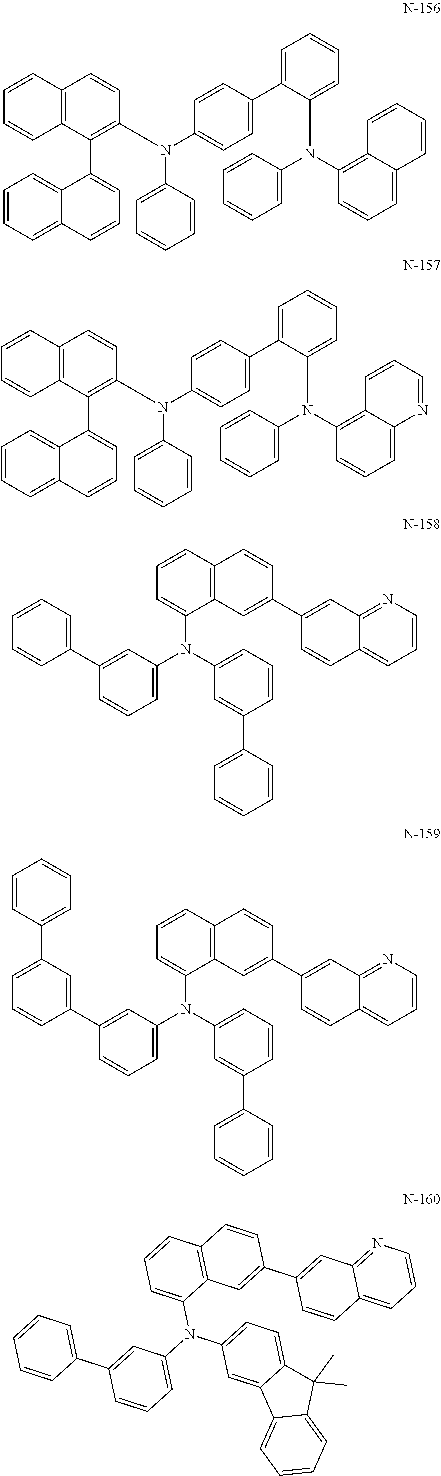 [SMILES: c1ccc(N(c2ccc(-c3ccccc3N(c3ccccc3)c3cccc4ccccc34)cc2)c2ccc3ccccc3c2-c2cccc3ccccc23)cc1 ; c1ccc(N(c2ccc(-c3ccccc3N(c3ccccc3)c3cccc4ncccc34)cc2)c2ccc3ccccc3c2-c2cccc3ccccc23)cc1 ; c1ccc(-c2cccc(N(c3cccc(-c4ccccc4)c3)c3cccc4ccc(-c5ccc6cccnc6c5)cc34)c2)cc1 ; c1ccc(-c2cccc(-c3cccc(N(c4cccc(-c5ccccc5)c4)c4cccc5ccc(-c6ccc7cccnc7c6)cc45)c3)c2)cc1 ; CC1(C)c2ccccc2-c2cc(N(c3cccc(-c4ccccc4)c3)c3cccc4ccc(-c5ccc6cccnc6c5)cc34)ccc21]

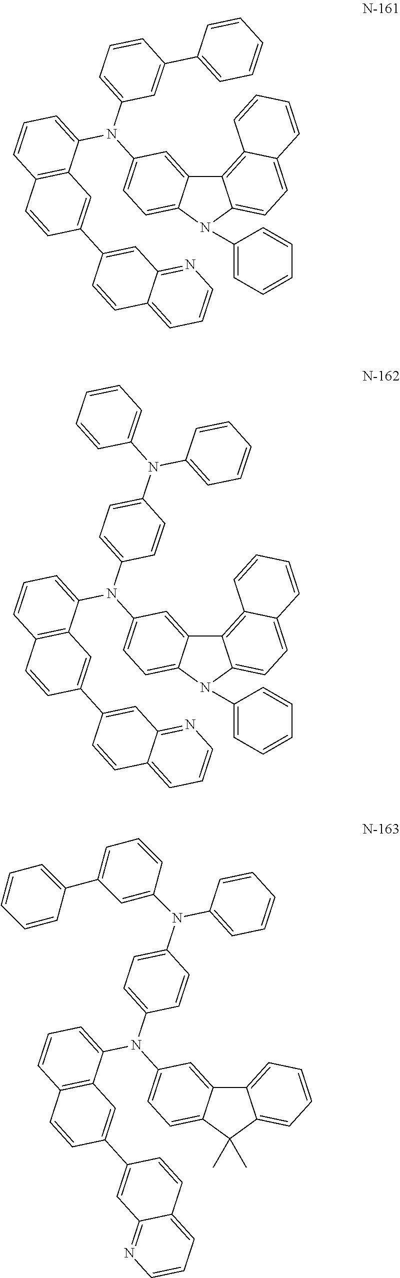 [SMILES: c1ccc(-c2cccc(N(c3ccc4c(c3)c3c5ccccc5ccc3n4-c3ccccc3)c3cccc4ccc(-c5ccc6cccnc6c5)cc34)c2)cc1 ; c1ccc(N(c2ccccc2)c2ccc(N(c3ccc4c(c3)c3c5ccccc5ccc3n4-c3ccccc3)c3cccc4ccc(-c5ccc6cccnc6c5)cc34)cc2)cc1 ; CC1(C)c2ccccc2-c2cc(N(c3ccc(N(c4ccccc4)c4cccc(-c5ccccc5)c4)cc3)c3cccc4ccc(-c5ccc6cccnc6c5)cc34)ccc21]

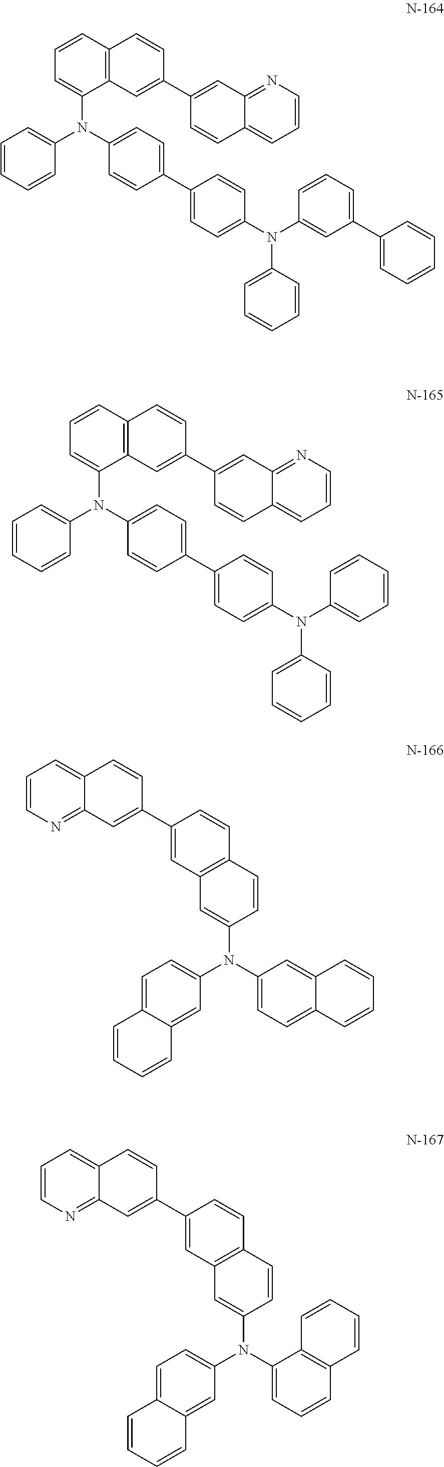 [SMILES: c1ccc(-c2cccc(N(c3ccccc3)c3ccc(-c4ccc(N(c5ccccc5)c5cccc6ccc(-c7ccc8cccnc8c7)cc56)cc4)cc3)c2)cc1 ; c1ccc(N(c2ccccc2)c2ccc(-c3ccc(N(c4ccccc4)c4cccc5ccc(-c6ccc7cccnc7c6)cc45)cc3)cc2)cc1 ; c1ccc2cc(N(c3ccc4ccccc4c3)c3ccc4ccc(-c5ccc6cccnc6c5)cc4c3)ccc2c1 ; c1ccc2cc(N(c3ccc4ccc(-c5ccc6cccnc6c5)cc4c3)c3cccc4ccccc34)ccc2c1]

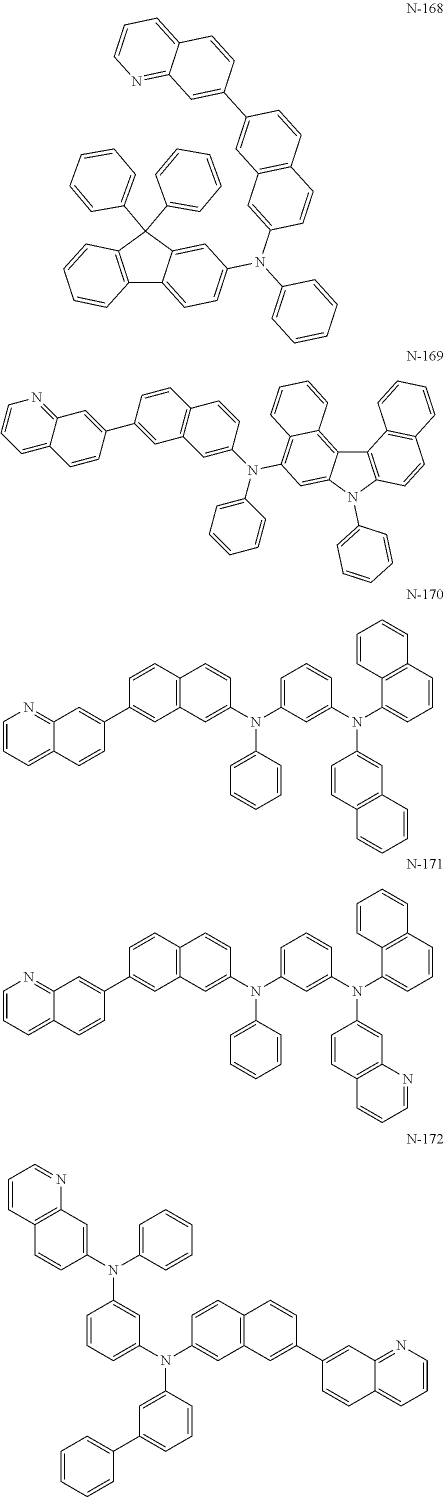

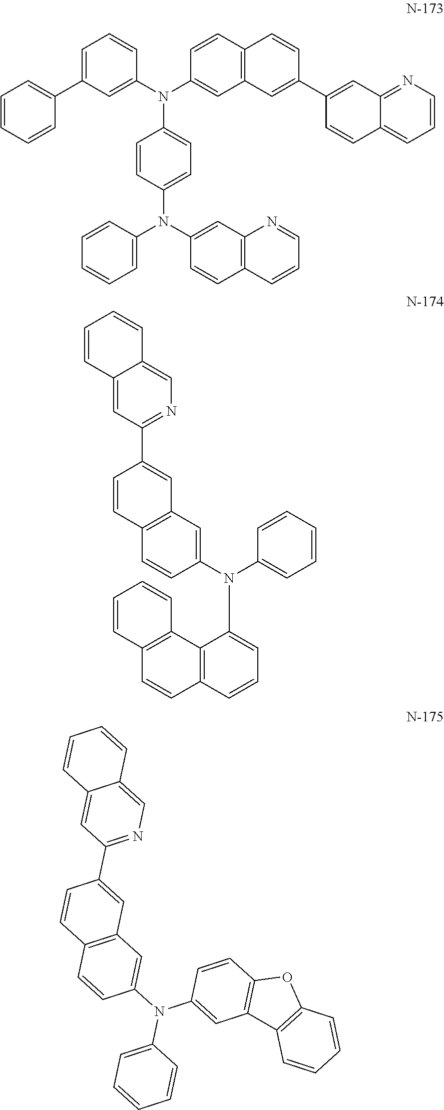

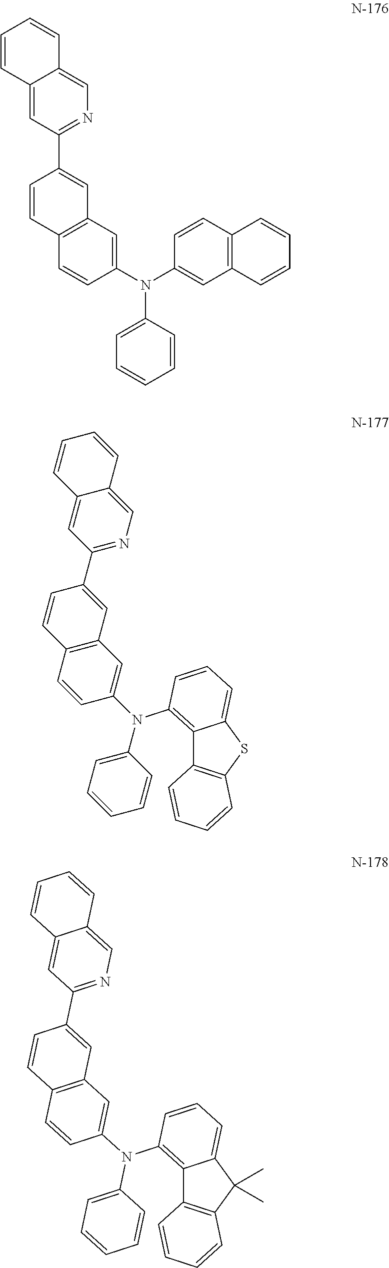 [SMILES: c1ccc(N(c2ccc3ccccc3c2)c2ccc3ccc(-c4cc5ccccc5cn4)cc3c2)cc1 ; c1ccc(N(c2ccc3ccc(-c4cc5ccccc5cn4)cc3c2)c2cccc3sc4ccccc4c23)cc1 ; CC1(C)c2ccccc2-c2c(N(c3ccccc3)c3ccc4ccc(-c5cc6ccccc6cn5)cc4c3)cccc21]

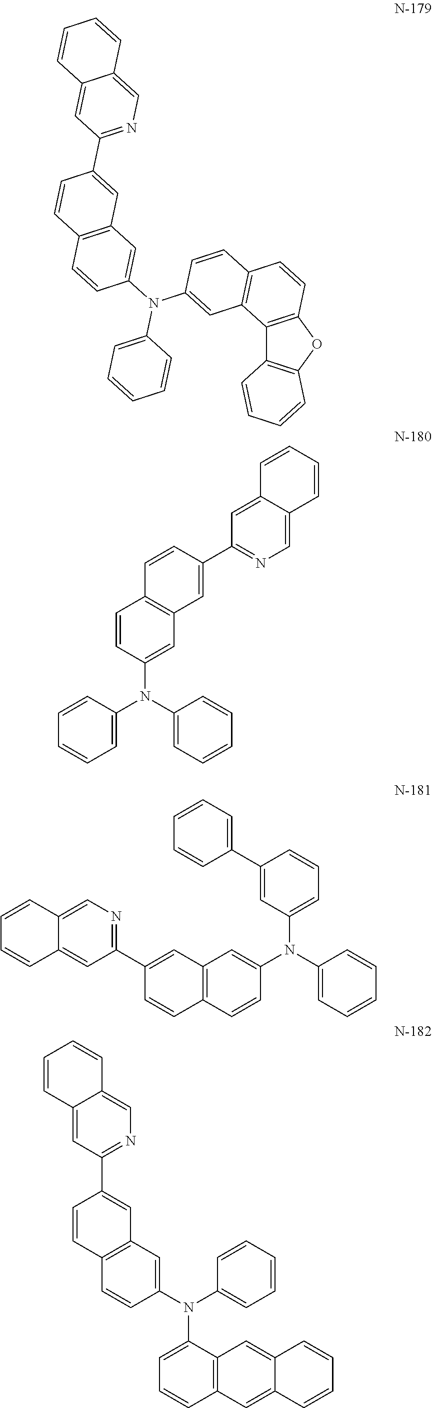

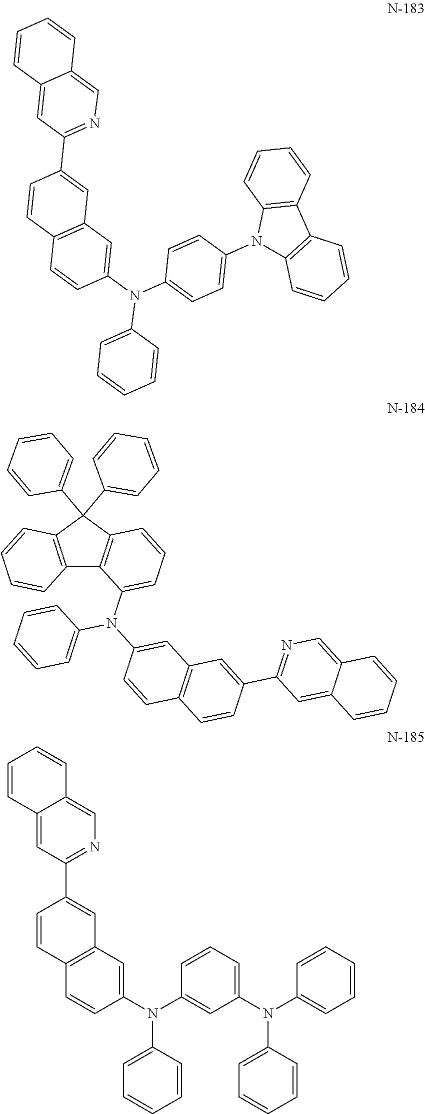

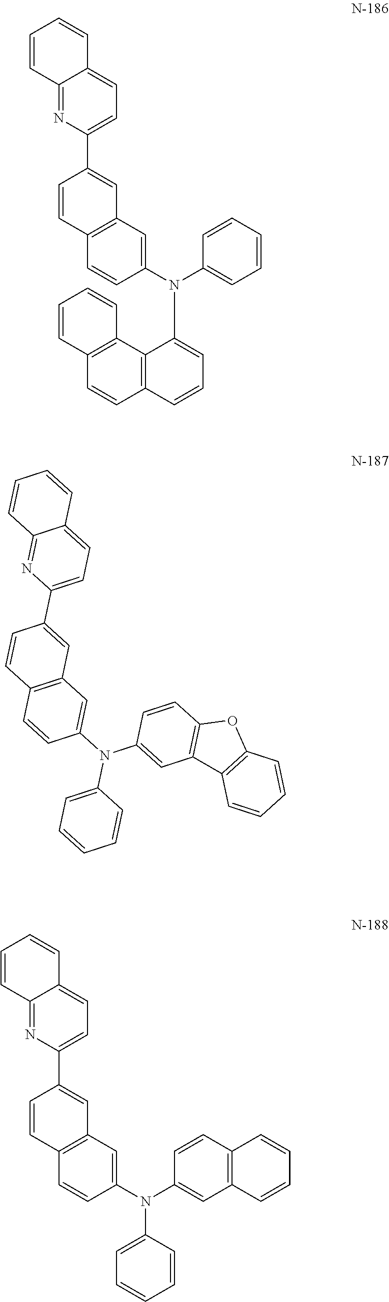 [SMILES: c1ccc(N(c2ccc3ccc(-c4ccc5ccccc5n4)cc3c2)c2cccc3ccc4ccccc4c23)cc1 ; c1ccc(N(c2ccc3ccc(-c4ccc5ccccc5n4)cc3c2)c2ccc3oc4ccccc4c3c2)cc1 ; c1ccc(N(c2ccc3ccccc3c2)c2ccc3ccc(-c4ccc5ccccc5n4)cc3c2)cc1]

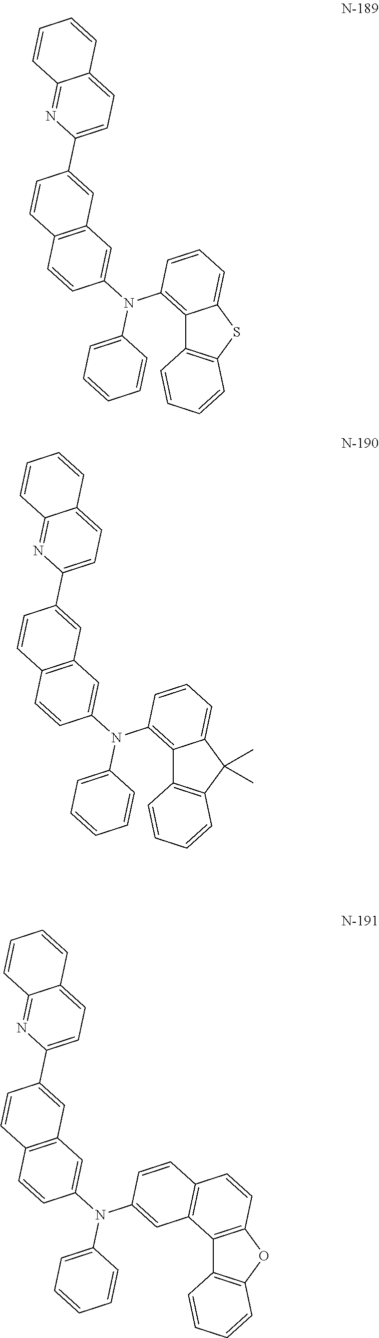 [SMILES: c1ccc(N(c2ccc3ccc(-c4ccc5ccccc5n4)cc3c2)c2cccc3sc4ccccc4c23)cc1 ; CC1(C)c2ccccc2-c2c(N(c3ccccc3)c3ccc4ccc(-c5ccc6ccccc6n5)cc4c3)cccc21 ; c1ccc(N(c2ccc3ccc(-c4ccc5ccccc5n4)cc3c2)c2ccc3ccc4oc5ccccc5c4c3c2)cc1]

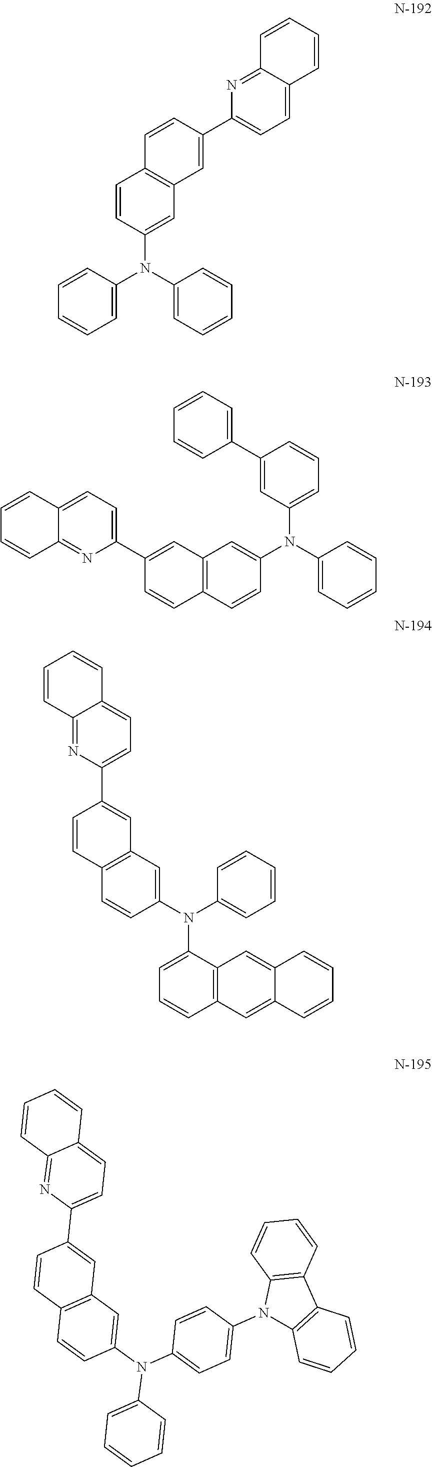 [SMILES: c1ccc(N(c2ccccc2)c2ccc3ccc(-c4ccc5ccccc5n4)cc3c2)cc1 ; c1ccc(-c2cccc(N(c3ccccc3)c3ccc4ccc(-c5ccc6ccccc6n5)cc4c3)c2)cc1 ; c1ccc(N(c2ccc3ccc(-c4ccc5ccccc5n4)cc3c2)c2cccc3cc4ccccc4cc23)cc1 ; c1ccc(N(c2ccc(-n3c4ccccc4c4ccccc43)cc2)c2ccc3ccc(-c4ccc5ccccc5n4)cc3c2)cc1]

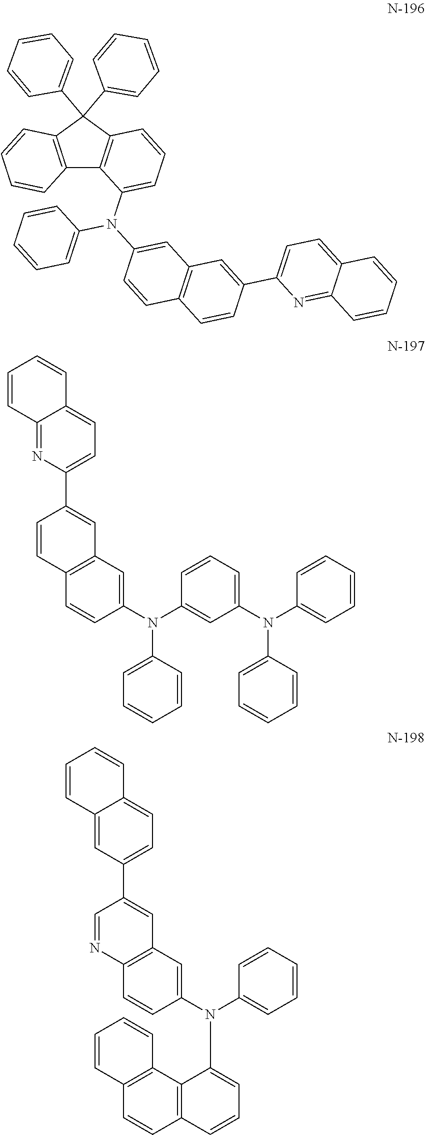 [SMILES: c1ccc(N(c2ccc3ccc(-c4ccc5ccccc5n4)cc3c2)c2cccc3c2-c2ccccc2C3(c2ccccc2)c2ccccc2)cc1 ; c1ccc(N(c2ccccc2)c2cccc(N(c3ccccc3)c3ccc4ccc(-c5ccc6ccccc6n5)cc4c3)c2)cc1 ; c1ccc(N(c2ccc3ncc(-c4ccc5ccccc5c4)cc3c2)c2cccc3ccc4ccccc4c23)cc1]

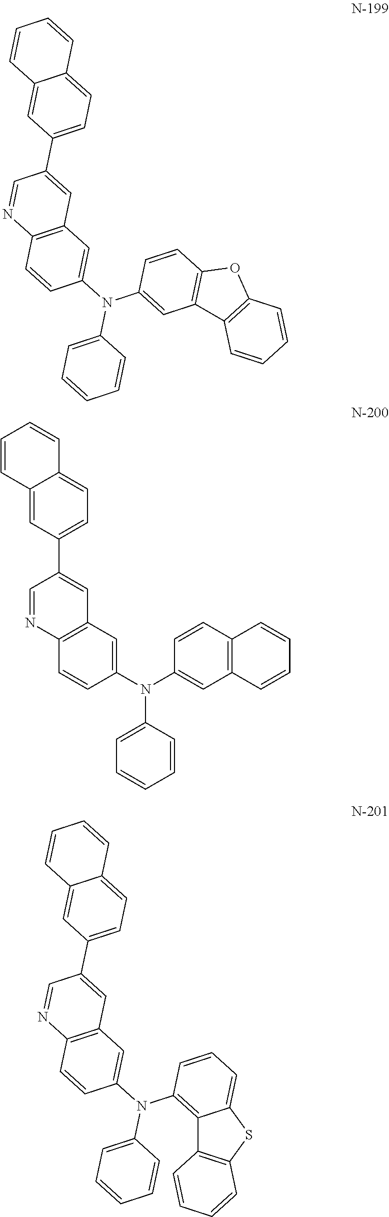 [SMILES: c1ccc(N(c2ccc3ncc(-c4ccc5ccccc5c4)cc3c2)c2ccc3oc4ccccc4c3c2)cc1 ; c1ccc(N(c2ccc3ccccc3c2)c2ccc3ncc(-c4ccc5ccccc5c4)cc3c2)cc1 ; c1ccc(N(c2ccc3ncc(-c4ccc5ccccc5c4)cc3c2)c2cccc3sc4ccccc4c23)cc1]

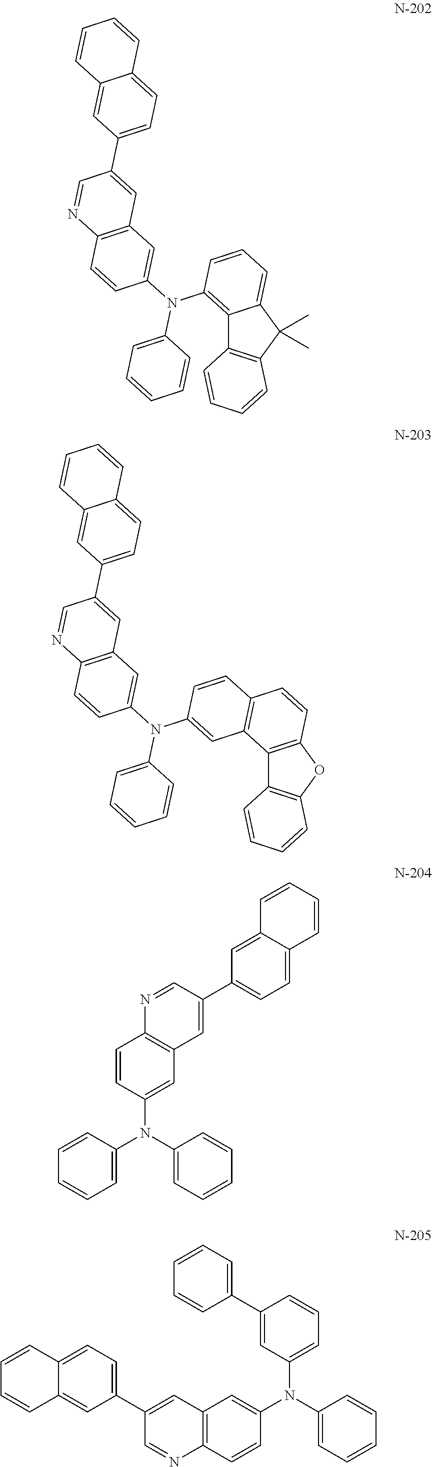

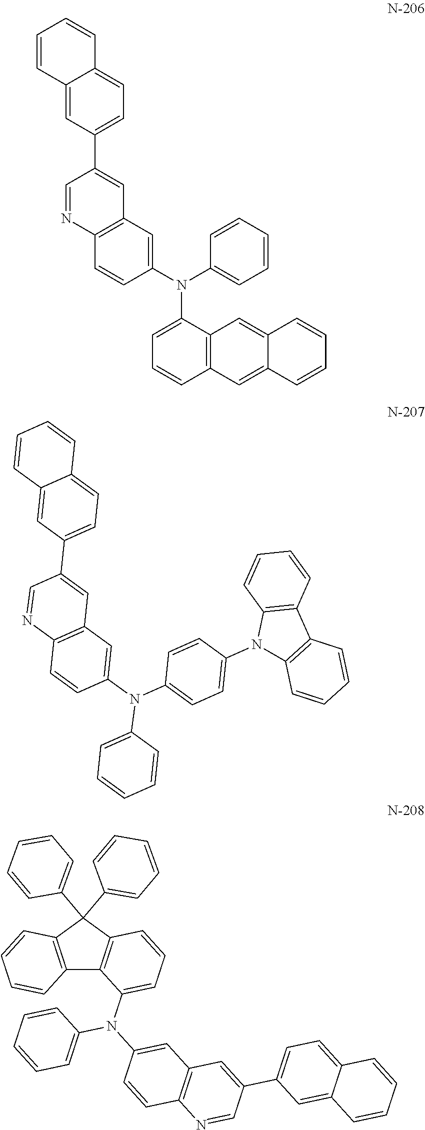

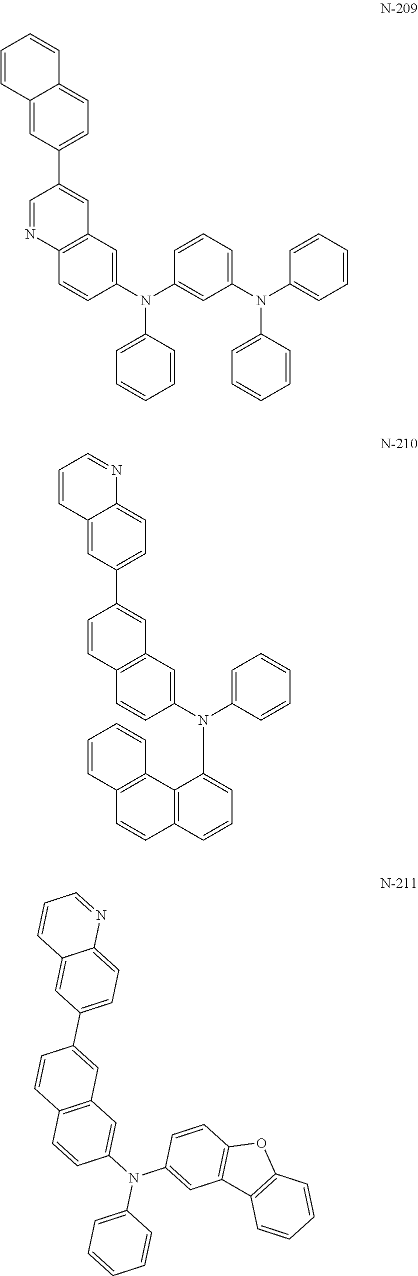 [SMILES: c1ccc(N(c2ccccc2)c2cccc(N(c3ccccc3)c3ccc4ncc(-c5ccc6ccccc6c5)cc4c3)c2)cc1 ; c1ccc(N(c2ccc3ccc(-c4ccc5ncccc5c4)cc3c2)c2cccc3ccc4ccccc4c23)cc1 ; c1ccc(N(c2ccc3ccc(-c4ccc5ncccc5c4)cc3c2)c2ccc3oc4ccccc4c3c2)cc1]

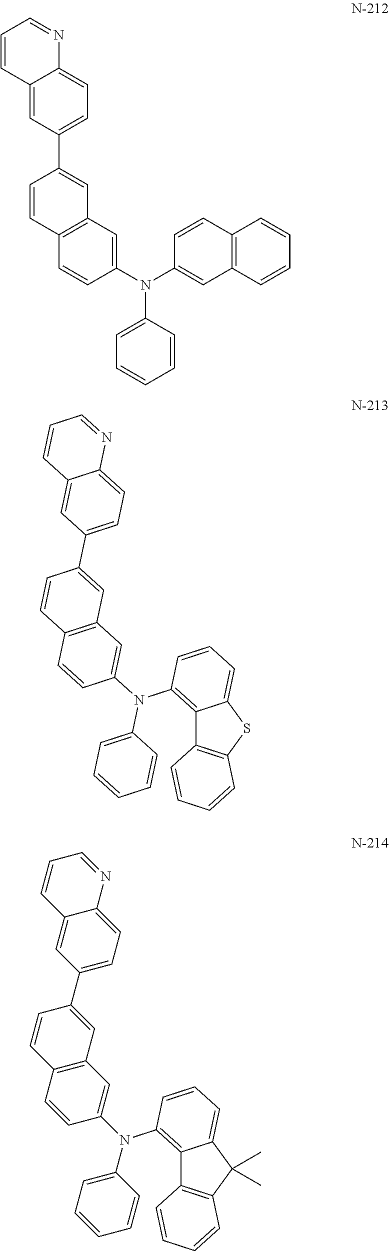 [SMILES: c1ccc(N(c2ccc3ccccc3c2)c2ccc3ccc(-c4ccc5ncccc5c4)cc3c2)cc1 ; c1ccc(N(c2ccc3ccc(-c4ccc5ncccc5c4)cc3c2)c2cccc3sc4ccccc4c23)cc1 ; CC1(C)c2ccccc2-c2c(N(c3ccccc3)c3ccc4ccc(-c5ccc6ncccc6c5)cc4c3)cccc21]

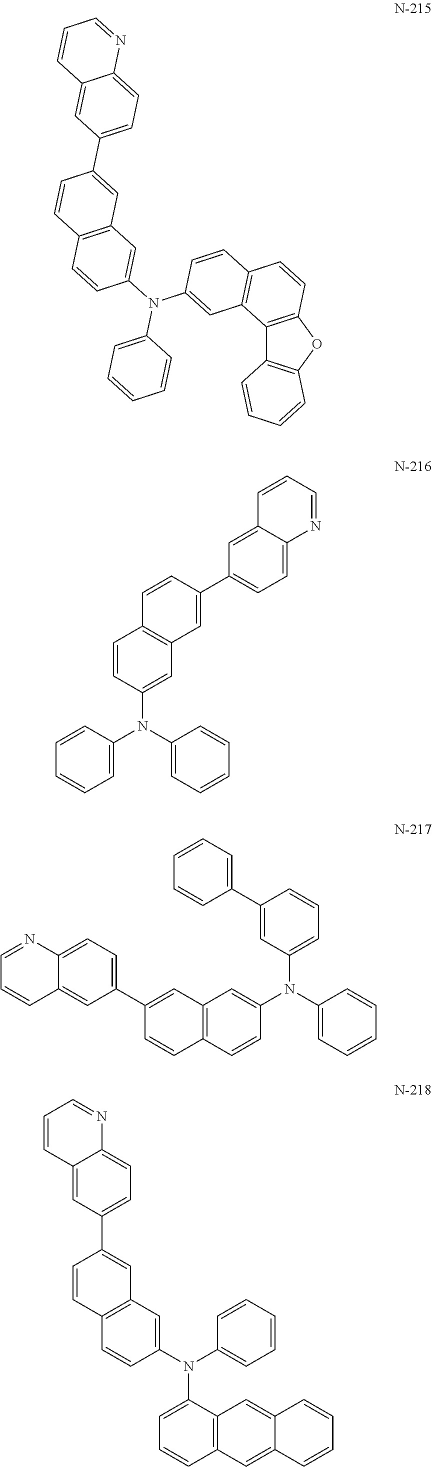

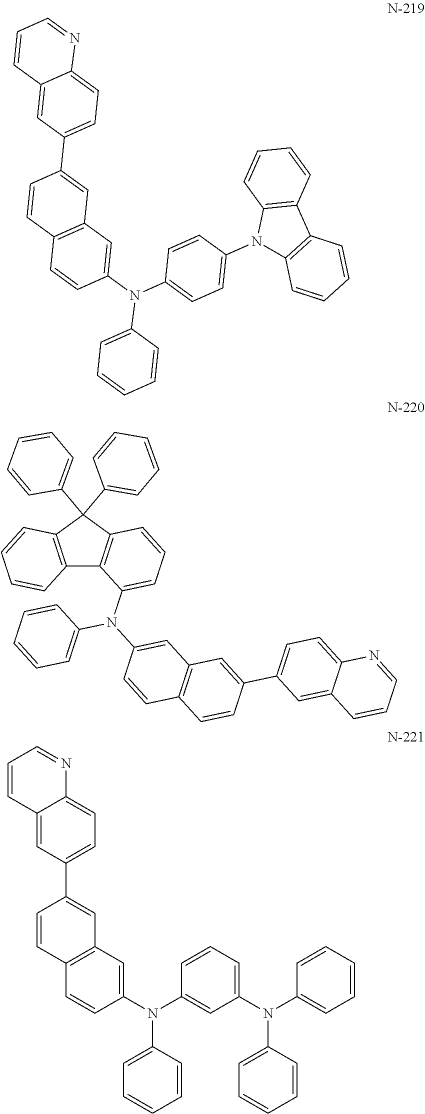

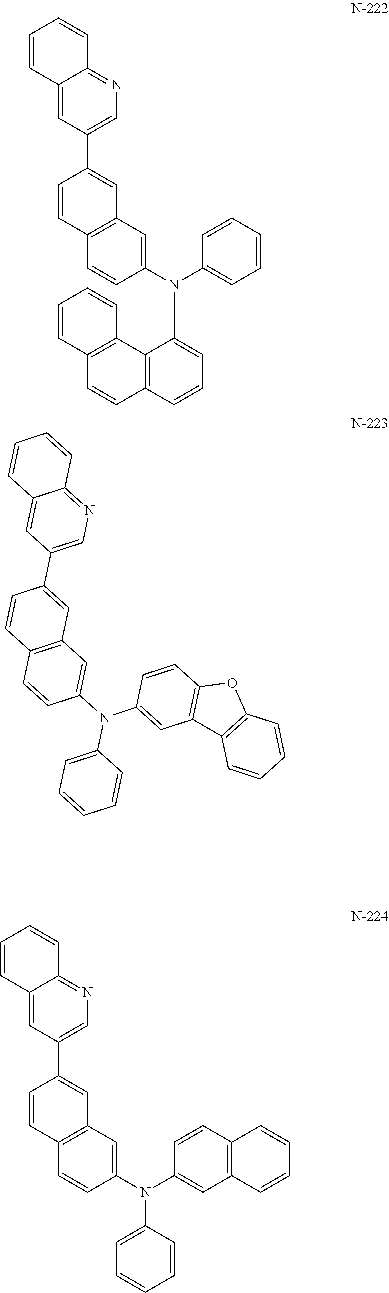 [SMILES: c1ccc(N(c2ccc3ccc(-c4cnc5ccccc5c4)cc3c2)c2cccc3ccc4ccccc4c23)cc1 ; c1ccc(N(c2ccc3ccc(-c4cnc5ccccc5c4)cc3c2)c2ccc3oc4ccccc4c3c2)cc1 ; c1ccc(N(c2ccc3ccccc3c2)c2ccc3ccc(-c4cnc5ccccc5c4)cc3c2)cc1]

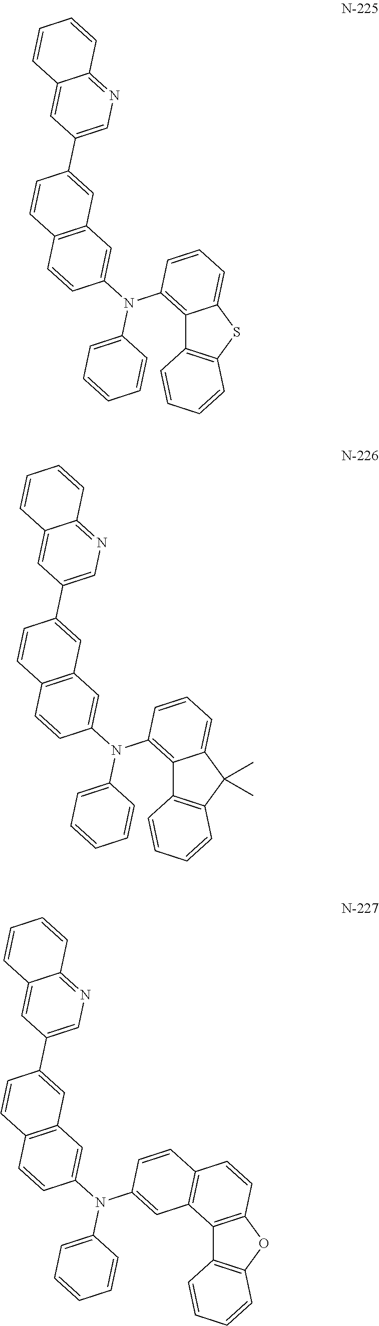 [SMILES: c1ccc(N(c2ccc3ccc(-c4cnc5ccccc5c4)cc3c2)c2cccc3sc4ccccc4c23)cc1 ; CC1(C)c2ccccc2-c2c(N(c3ccccc3)c3ccc4ccc(-c5cnc6ccccc6c5)cc4c3)cccc21 ; c1ccc(N(c2ccc3ccc(-c4cnc5ccccc5c4)cc3c2)c2ccc3ccc4oc5ccccc5c4c3c2)cc1]

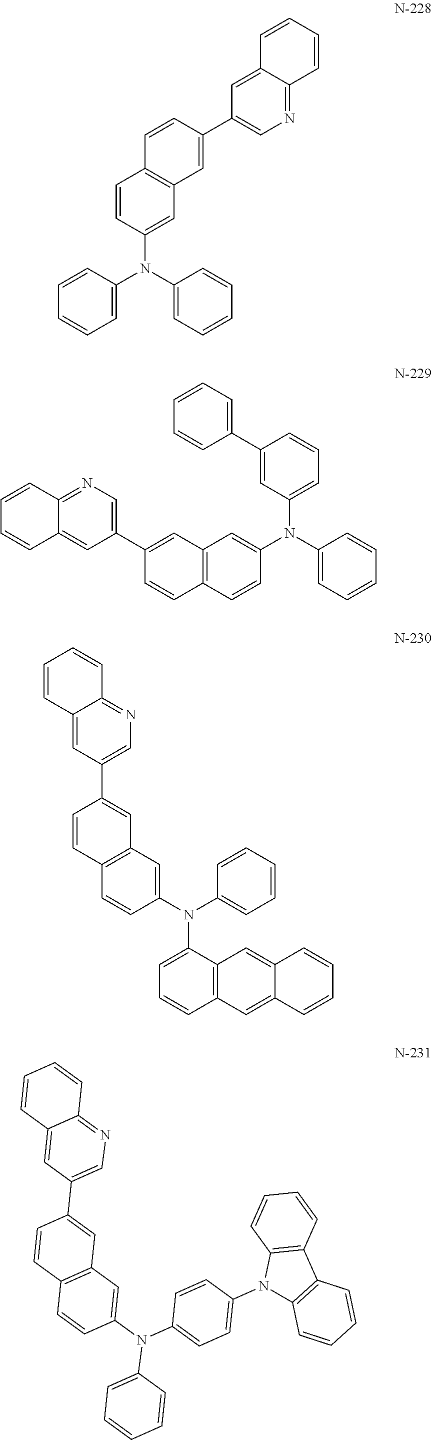 [SMILES: c1ccc(N(c2ccccc2)c2ccc3ccc(-c4cnc5ccccc5c4)cc3c2)cc1 ; c1ccc(-c2cccc(N(c3ccccc3)c3ccc4ccc(-c5cnc6ccccc6c5)cc4c3)c2)cc1 ; c1ccc(N(c2ccc3ccc(-c4cnc5ccccc5c4)cc3c2)c2cccc3cc4ccccc4cc23)cc1 ; c1ccc(N(c2ccc(-n3c4ccccc4c4ccccc43)cc2)c2ccc3ccc(-c4cnc5ccccc5c4)cc3c2)cc1]

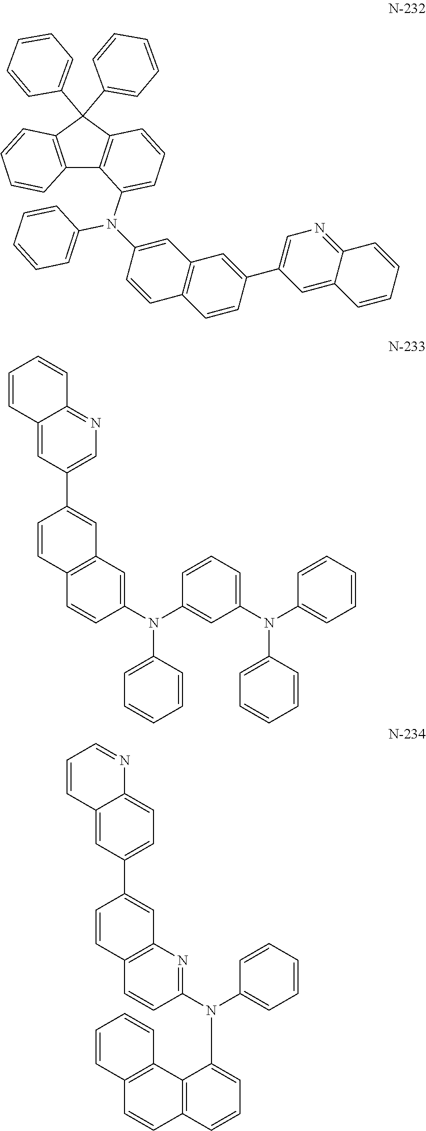 [SMILES: c1ccc(N(c2ccc3ccc(-c4cnc5ccccc5c4)cc3c2)c2cccc3c2-c2ccccc2C3(c2ccccc2)c2ccccc2)cc1 ; c1ccc(N(c2ccccc2)c2cccc(N(c3ccccc3)c3ccc4ccc(-c5cnc6ccccc6c5)cc4c3)c2)cc1 ; c1ccc(N(c2ccc3ccc(-c4ccc5ncccc5c4)cc3n2)c2cccc3ccc4ccccc4c23)cc1]

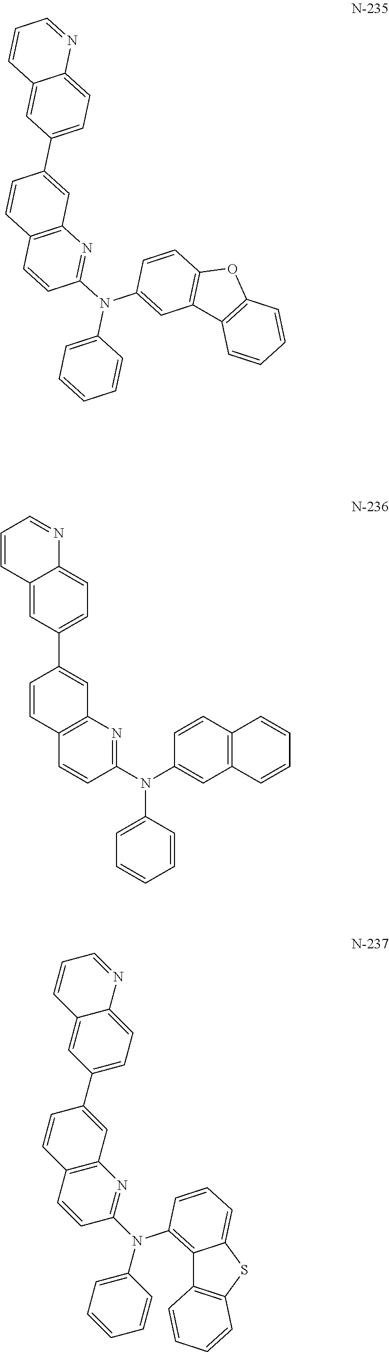 [SMILES: c1ccc(N(c2ccc3oc4ccccc4c3c2)c2ccc3ccc(-c4ccc5ncccc5c4)cc3n2)cc1 ; c1ccc(N(c2ccc3ccccc3c2)c2ccc3ccc(-c4ccc5ncccc5c4)cc3n2)cc1 ; c1ccc(N(c2ccc3ccc(-c4ccc5ncccc5c4)cc3n2)c2cccc3sc4ccccc4c23)cc1]

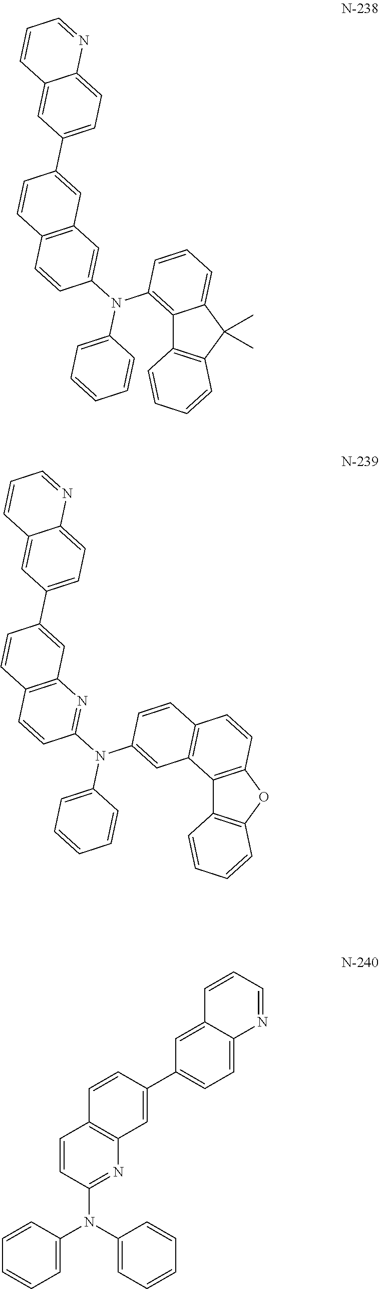 [SMILES: CC1(C)c2ccccc2-c2c(N(c3ccccc3)c3ccc4ccc(-c5ccc6ncccc6c5)cc4c3)cccc21 ; c1ccc(N(c2ccc3ccc4oc5ccccc5c4c3c2)c2ccc3ccc(-c4ccc5ncccc5c4)cc3n2)cc1 ; c1ccc(N(c2ccccc2)c2ccc3ccc(-c4ccc5ncccc5c4)cc3n2)cc1]

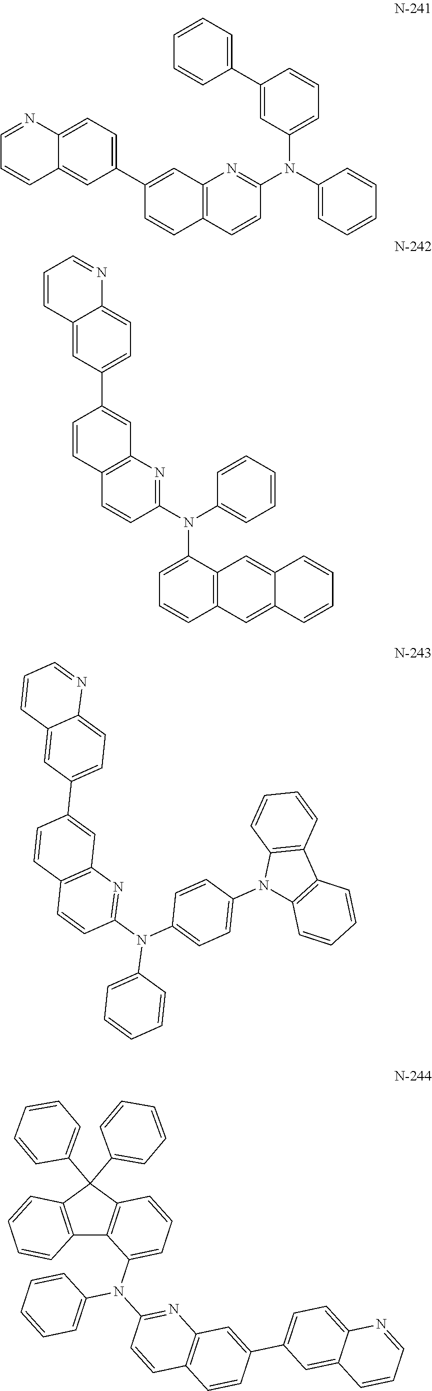 [SMILES: c1ccc(-c2cccc(N(c3ccccc3)c3ccc4ccc(-c5ccc6ncccc6c5)cc4n3)c2)cc1 ; c1ccc(N(c2ccc3ccc(-c4ccc5ncccc5c4)cc3n2)c2cccc3cc4ccccc4cc23)cc1 ; c1ccc(N(c2ccc(-n3c4ccccc4c4ccccc43)cc2)c2ccc3ccc(-c4ccc5ncccc5c4)cc3n2)cc1 ; c1ccc(N(c2ccc3ccc(-c4ccc5ncccc5c4)cc3n2)c2cccc3c2-c2ccccc2C3(c2ccccc2)c2ccccc2)cc1]

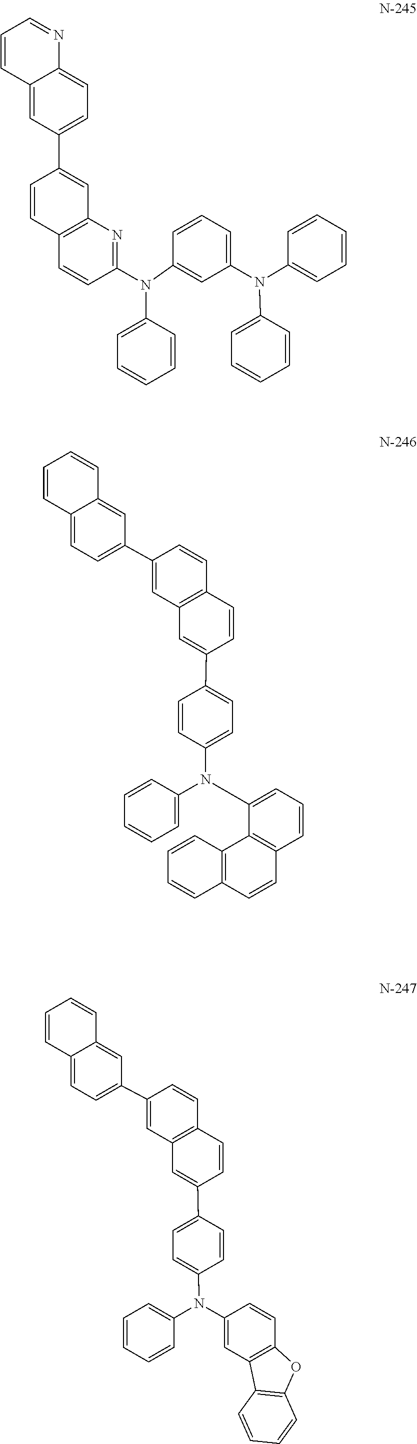 [SMILES: c1ccc(N(c2ccccc2)c2cccc(N(c3ccccc3)c3ccc4ccc(-c5ccc6ncccc6c5)cc4n3)c2)cc1 ; c1ccc(N(c2ccc(-c3ccc4ccc(-c5ccc6ccccc6c5)cc4c3)cc2)c2cccc3ccc4ccccc4c23)cc1 ; c1ccc(N(c2ccc(-c3ccc4ccc(-c5ccc6ccccc6c5)cc4c3)cc2)c2ccc3oc4ccccc4c3c2)cc1]

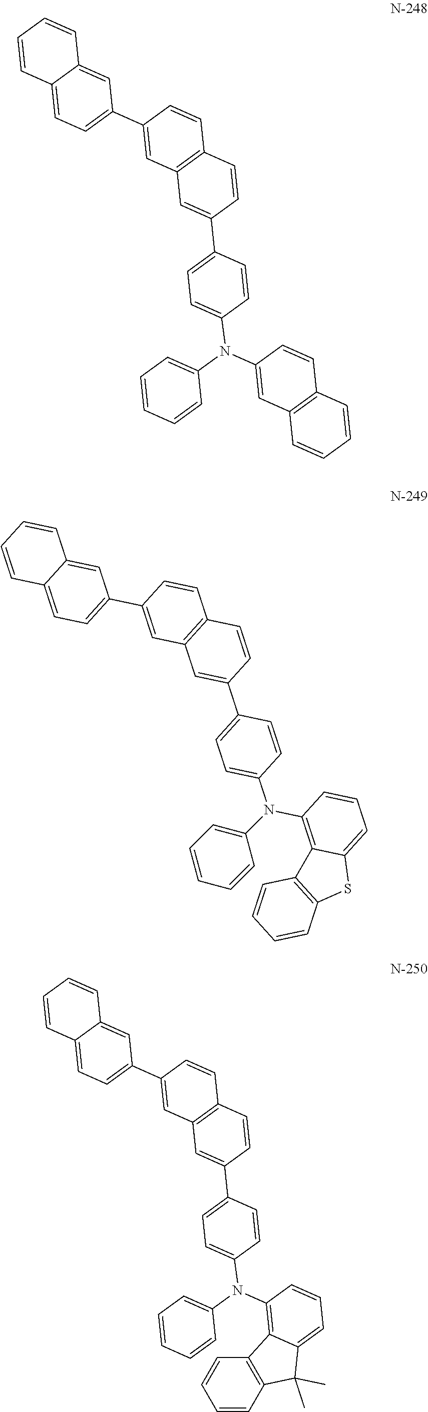 [SMILES: c1ccc(N(c2ccc(-c3ccc4ccc(-c5ccc6ccccc6c5)cc4c3)cc2)c2ccc3ccccc3c2)cc1 ; c1ccc(N(c2ccc(-c3ccc4ccc(-c5ccc6ccccc6c5)cc4c3)cc2)c2cccc3sc4ccccc4c23)cc1 ; CC1(C)c2ccccc2-c2c(N(c3ccccc3)c3ccc(-c4ccc5ccc(-c6ccc7ccccc7c6)cc5c4)cc3)cccc21]

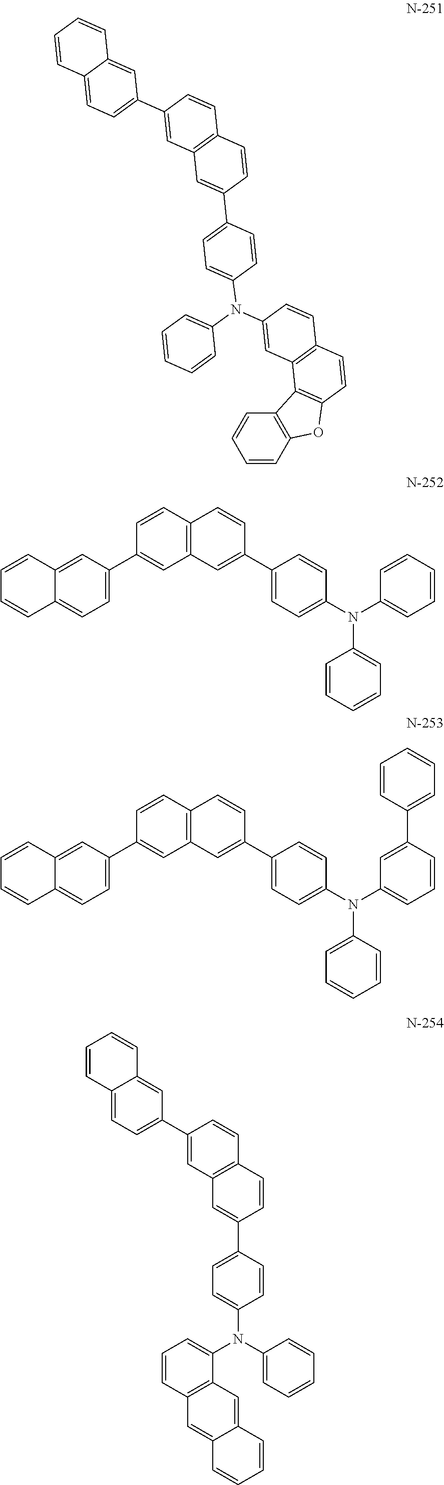 [SMILES: c1ccc(N(c2ccc(-c3ccc4ccc(-c5ccc6ccccc6c5)cc4c3)cc2)c2ccc3ccc4oc5ccccc5c4c3c2)cc1 ; c1ccc(N(c2ccccc2)c2ccc(-c3ccc4ccc(-c5ccc6ccccc6c5)cc4c3)cc2)cc1 ; c1ccc(-c2cccc(N(c3ccccc3)c3ccc(-c4ccc5ccc(-c6ccc7ccccc7c6)cc5c4)cc3)c2)cc1 ; c1ccc(N(c2ccc(-c3ccc4ccc(-c5ccc6ccccc6c5)cc4c3)cc2)c2cccc3cc4ccccc4cc23)cc1]

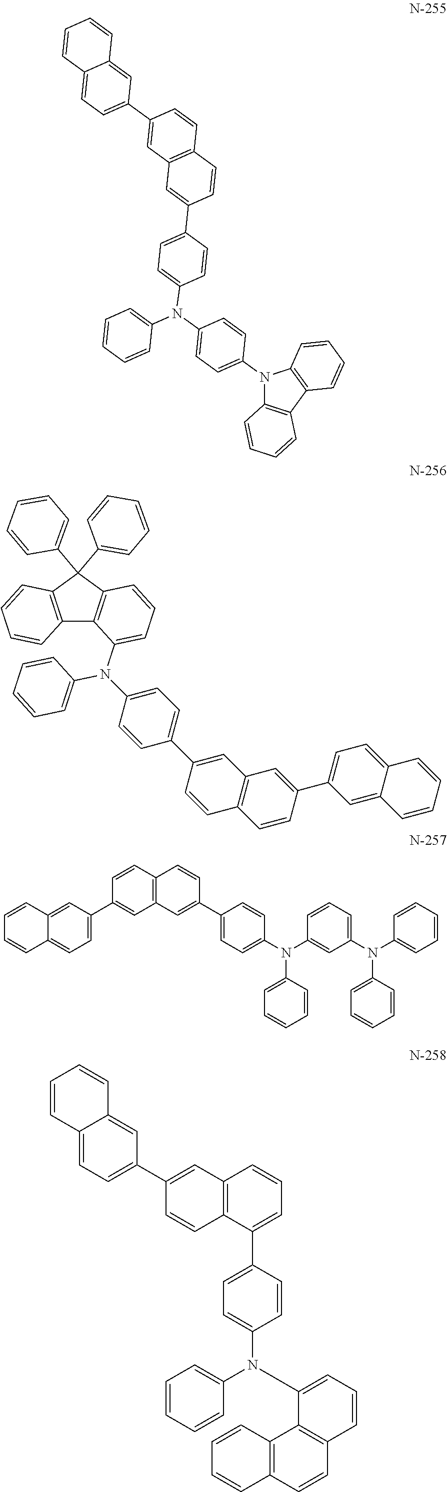 [SMILES: c1ccc(N(c2ccc(-c3ccc4ccc(-c5ccc6ccccc6c5)cc4c3)cc2)c2ccc(-n3c4ccccc4c4ccccc43)cc2)cc1 ; c1ccc(N(c2ccc(-c3ccc4ccc(-c5ccc6ccccc6c5)cc4c3)cc2)c2cccc3c2-c2ccccc2C3(c2ccccc2)c2ccccc2)cc1 ; c1ccc(N(c2ccccc2)c2cccc(N(c3ccccc3)c3ccc(-c4ccc5ccc(-c6ccc7ccccc7c6)cc5c4)cc3)c2)cc1 ; c1ccc(N(c2ccc(-c3cccc4cc(-c5ccc6ccccc6c5)ccc34)cc2)c2cccc3ccc4ccccc4c23)cc1]

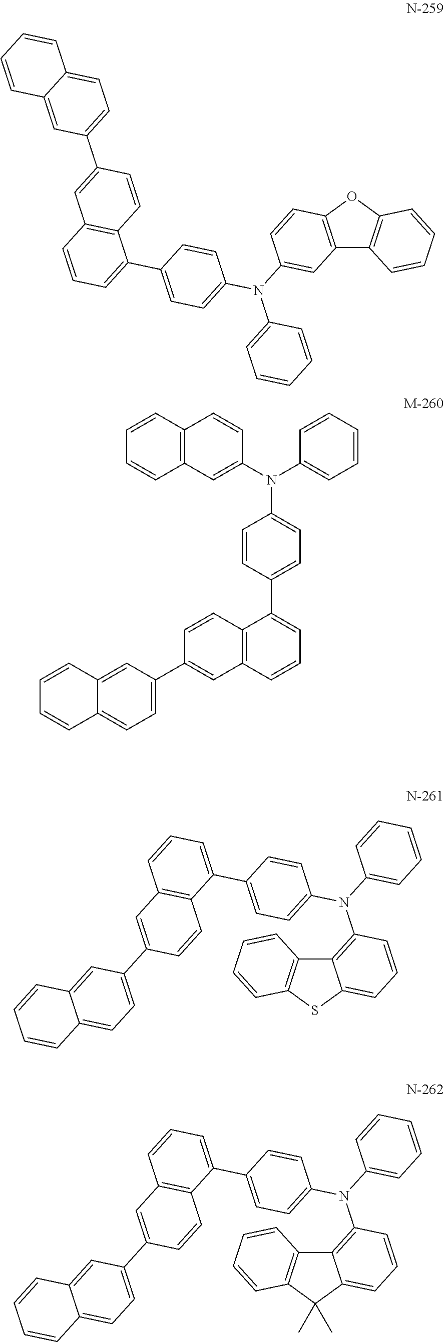

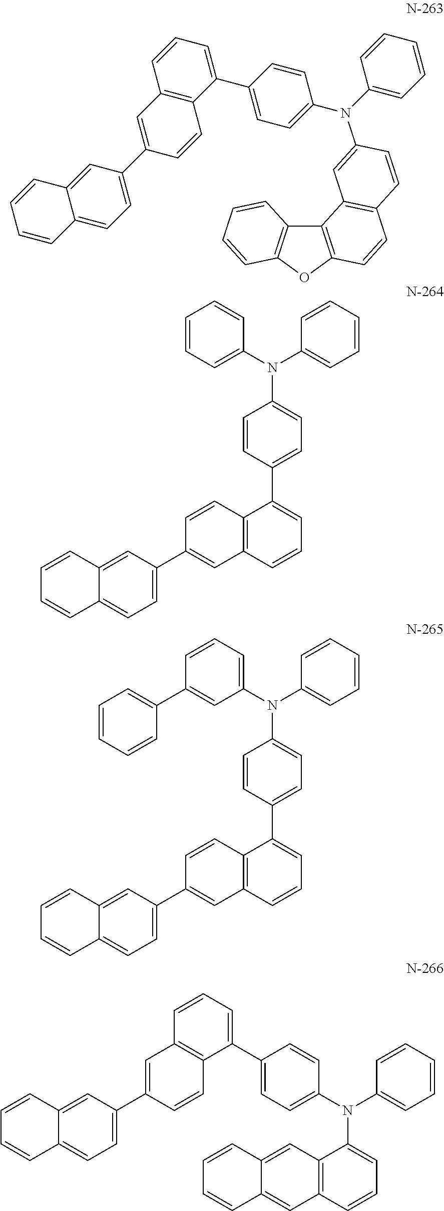

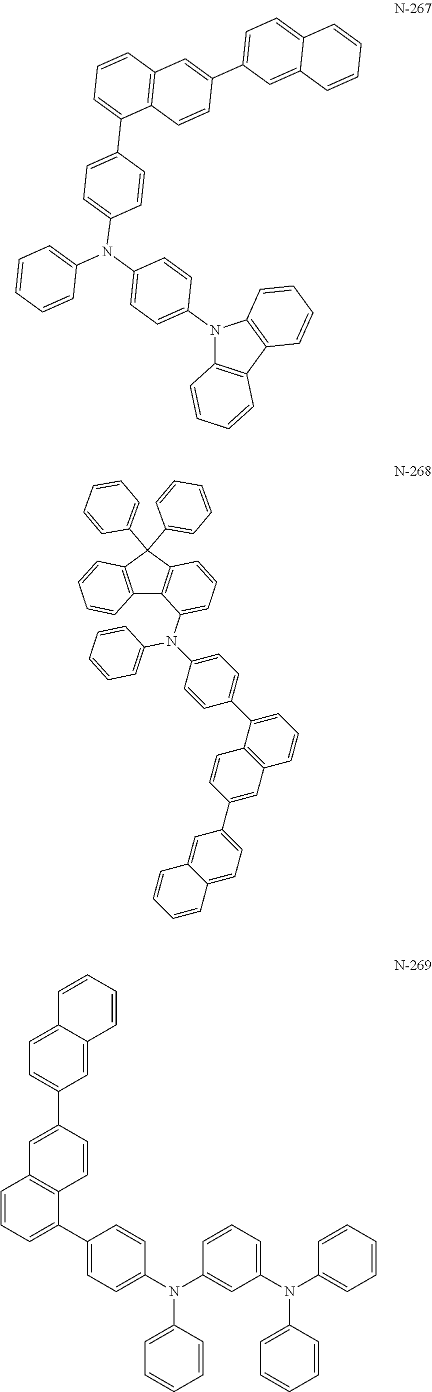 [SMILES: c1ccc(N(c2ccc(-c3cccc4cc(-c5ccc6ccccc6c5)ccc34)cc2)c2ccc(-n3c4ccccc4c4ccccc43)cc2)cc1 ; c1ccc(N(c2ccc(-c3cccc4cc(-c5ccc6ccccc6c5)ccc34)cc2)c2cccc3c2-c2ccccc2C3(c2ccccc2)c2ccccc2)cc1 ; c1ccc(N(c2ccccc2)c2cccc(N(c3ccccc3)c3ccc(-c4cccc5cc(-c6ccc7ccccc7c6)ccc45)cc3)c2)cc1]

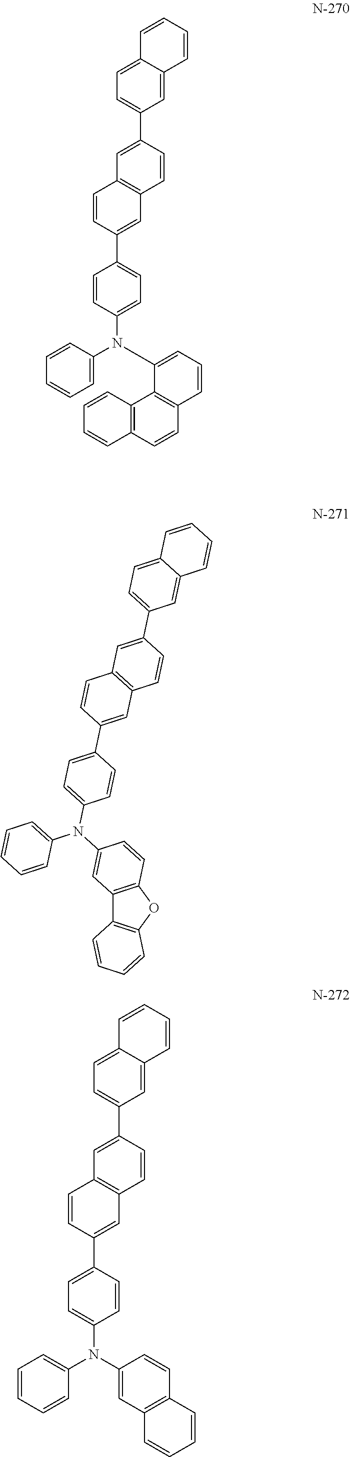

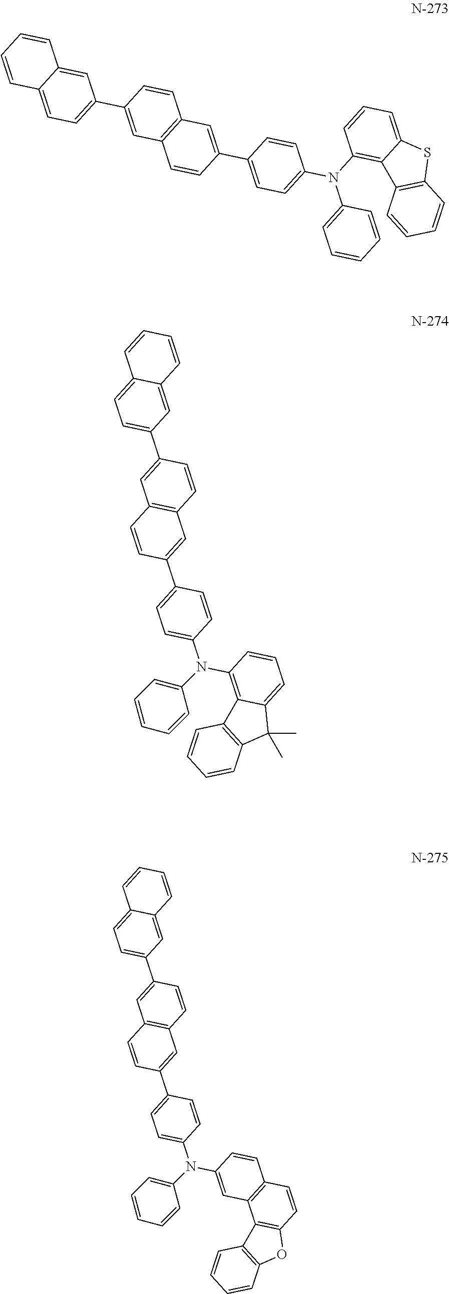 [SMILES: c1ccc(N(c2ccc(-c3ccc4cc(-c5ccc6ccccc6c5)ccc4c3)cc2)c2cccc3sc4ccccc4c23)cc1 ; CC1(C)c2ccccc2-c2c(N(c3ccccc3)c3ccc(-c4ccc5cc(-c6ccc7ccccc7c6)ccc5c4)cc3)cccc21 ; c1ccc(N(c2ccc(-c3ccc4cc(-c5ccc6ccccc6c5)ccc4c3)cc2)c2ccc3ccc4oc5ccccc5c4c3c2)cc1]

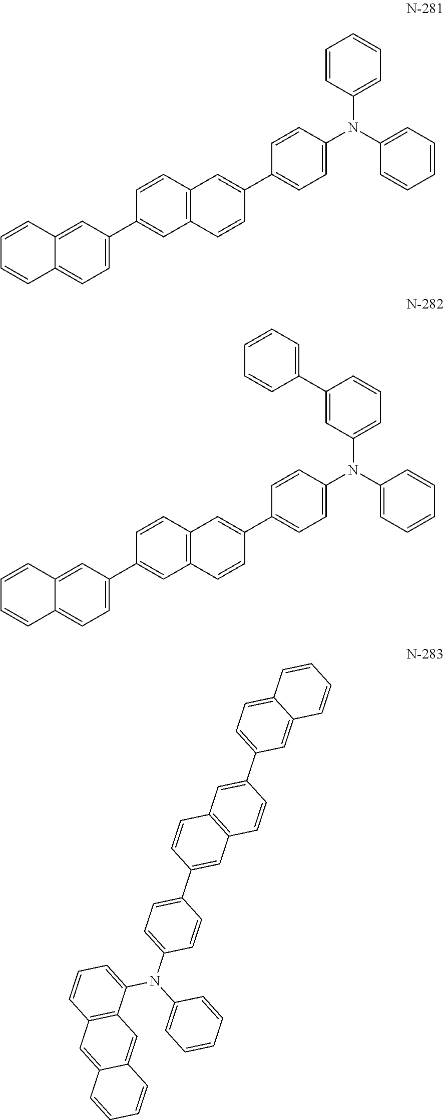 [SMILES: c1ccc(N(c2ccccc2)c2ccc(-c3ccc4cc(-c5ccc6ccccc6c5)ccc4c3)cc2)cc1 ; c1ccc(-c2cccc(N(c3ccccc3)c3ccc(-c4ccc5cc(-c6ccc7ccccc7c6)ccc5c4)cc3)c2)cc1 ; c1ccc(N(c2ccc(-c3ccc4cc(-c5ccc6ccccc6c5)ccc4c3)cc2)c2cccc3cc4ccccc4cc23)cc1]

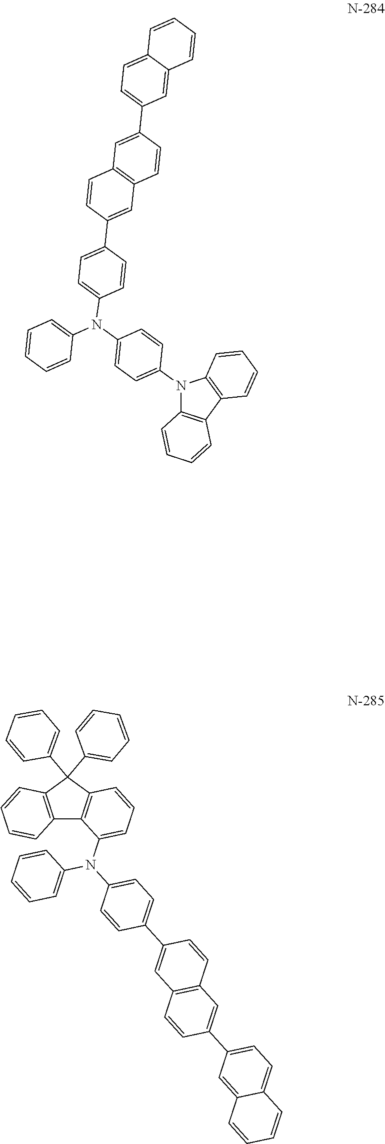 [SMILES: c1ccc(N(c2ccc(-c3ccc4cc(-c5ccc6ccccc6c5)ccc4c3)cc2)c2ccc(-n3c4ccccc4c4ccccc43)cc2)cc1 ; c1ccc(N(c2ccc(-c3ccc4cc(-c5ccc6ccccc6c5)ccc4c3)cc2)c2cccc3c2-c2ccccc2C3(c2ccccc2)c2ccccc2)cc1]

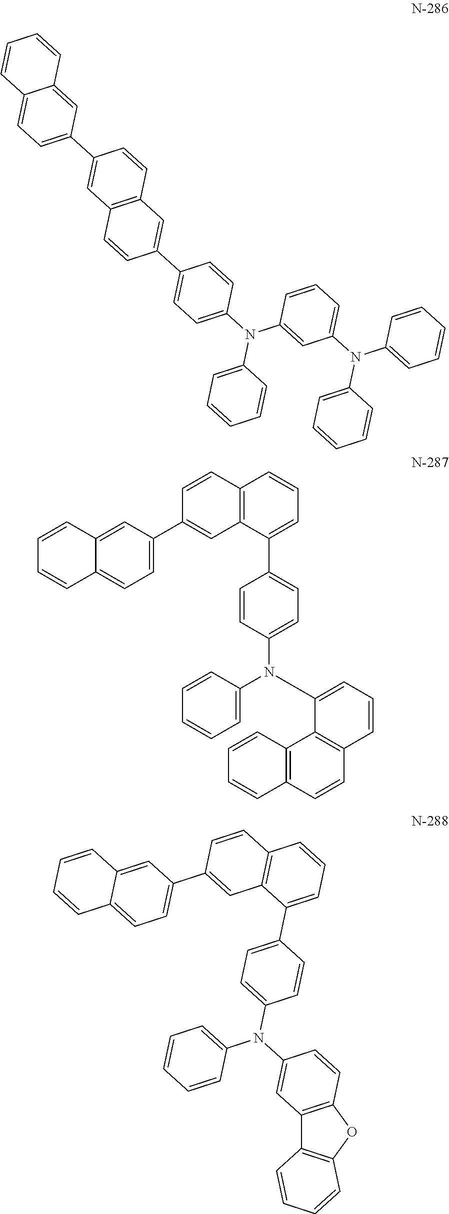

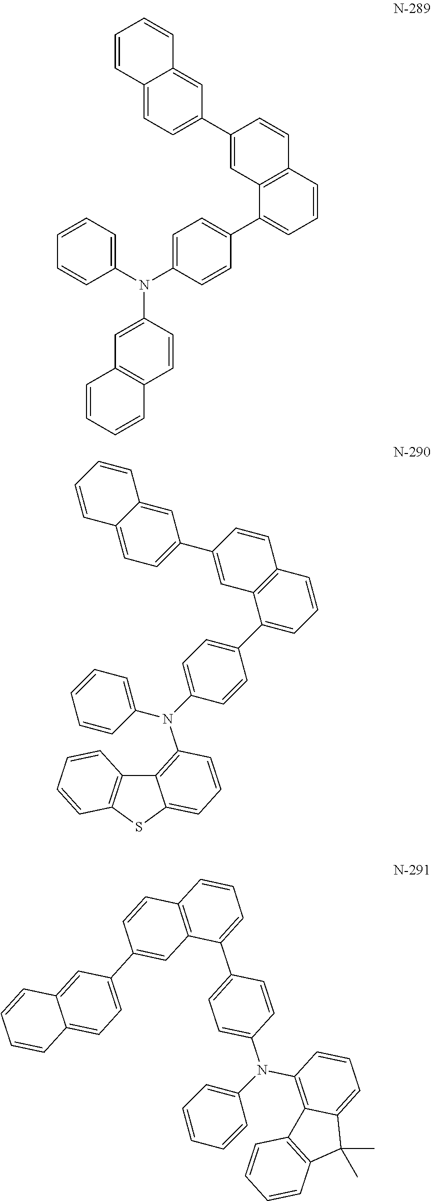

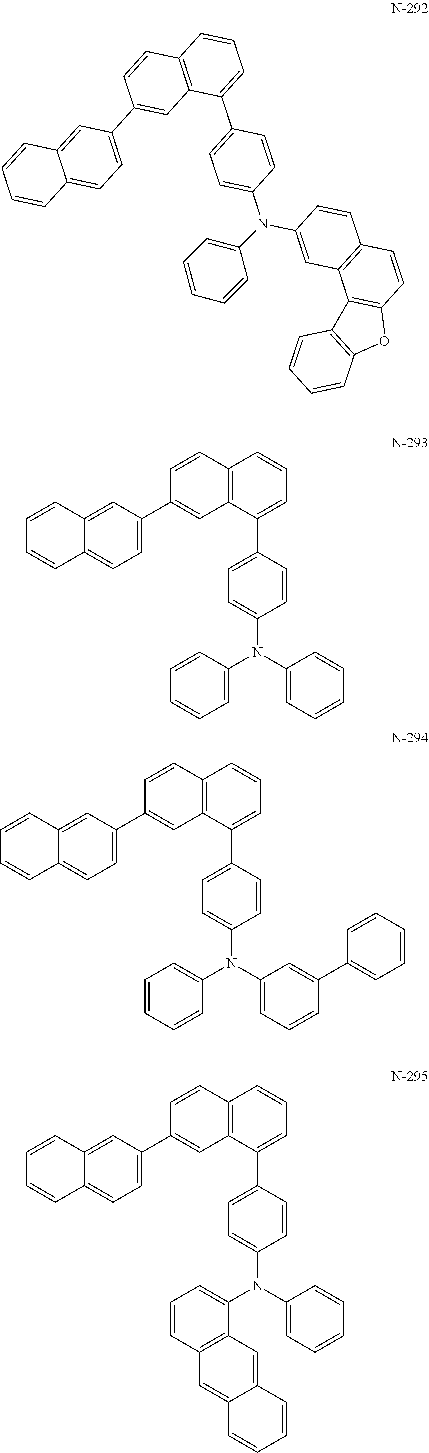 [SMILES: c1ccc(N(c2ccc(-c3cccc4ccc(-c5ccc6ccccc6c5)cc34)cc2)c2ccc3ccc4oc5ccccc5c4c3c2)cc1 ; c1ccc(N(c2ccccc2)c2ccc(-c3cccc4ccc(-c5ccc6ccccc6c5)cc34)cc2)cc1 ; c1ccc(-c2cccc(N(c3ccccc3)c3ccc(-c4cccc5ccc(-c6ccc7ccccc7c6)cc45)cc3)c2)cc1 ; c1ccc(N(c2ccc(-c3cccc4ccc(-c5ccc6ccccc6c5)cc34)cc2)c2cccc3cc4ccccc4cc23)cc1]

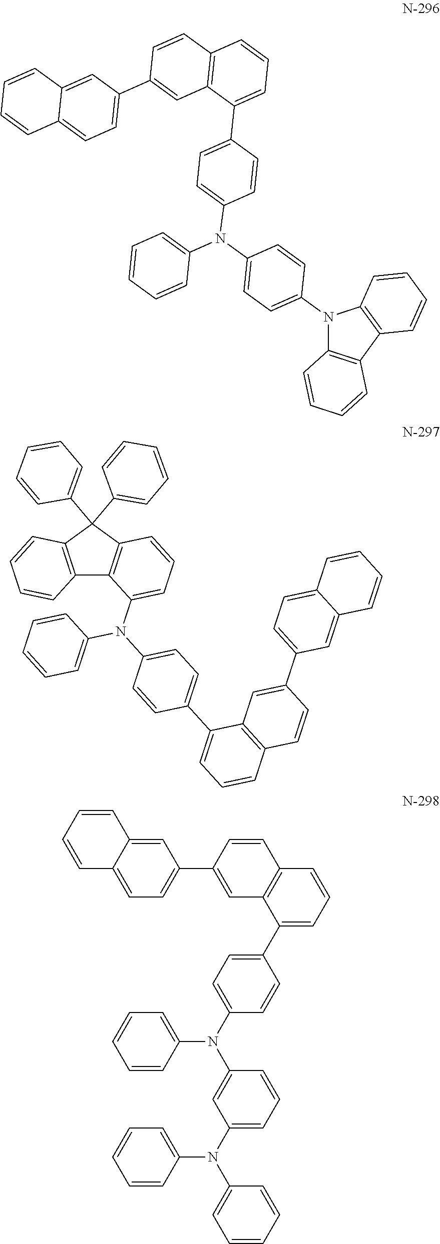 [SMILES: c1ccc(N(c2ccc(-c3cccc4ccc(-c5ccc6ccccc6c5)cc34)cc2)c2ccc(-n3c4ccccc4c4ccccc43)cc2)cc1 ; c1ccc(N(c2ccc(-c3cccc4ccc(-c5ccc6ccccc6c5)cc34)cc2)c2cccc3c2-c2ccccc2C3(c2ccccc2)c2ccccc2)cc1 ; c1ccc(N(c2ccccc2)c2cccc(N(c3ccccc3)c3ccc(-c4cccc5ccc(-c6ccc7ccccc7c6)cc45)cc3)c2)cc1]

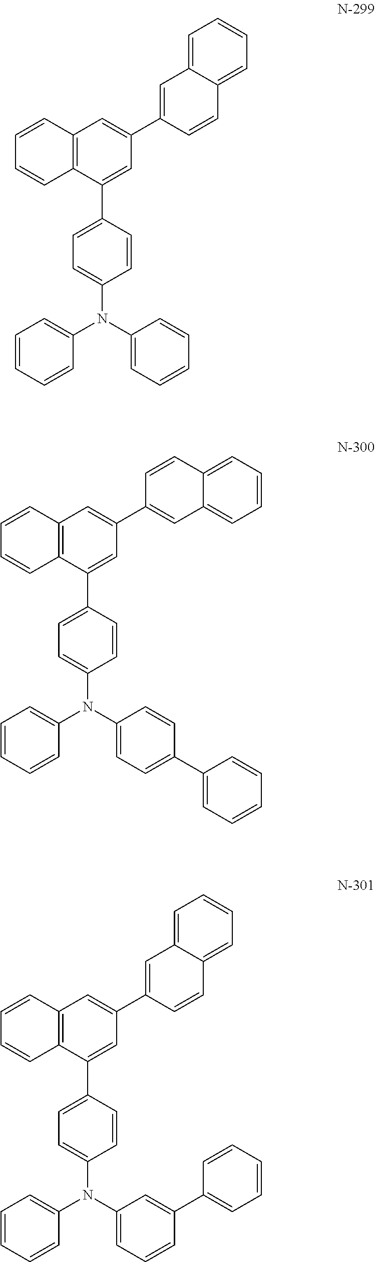 [SMILES: c1ccc(N(c2ccccc2)c2ccc(-c3cc(-c4ccc5ccccc5c4)cc4ccccc34)cc2)cc1 ; c1ccc(-c2ccc(N(c3ccccc3)c3ccc(-c4cc(-c5ccc6ccccc6c5)cc5ccccc45)cc3)cc2)cc1 ; c1ccc(-c2cccc(N(c3ccccc3)c3ccc(-c4cc(-c5ccc6ccccc6c5)cc5ccccc45)cc3)c2)cc1]

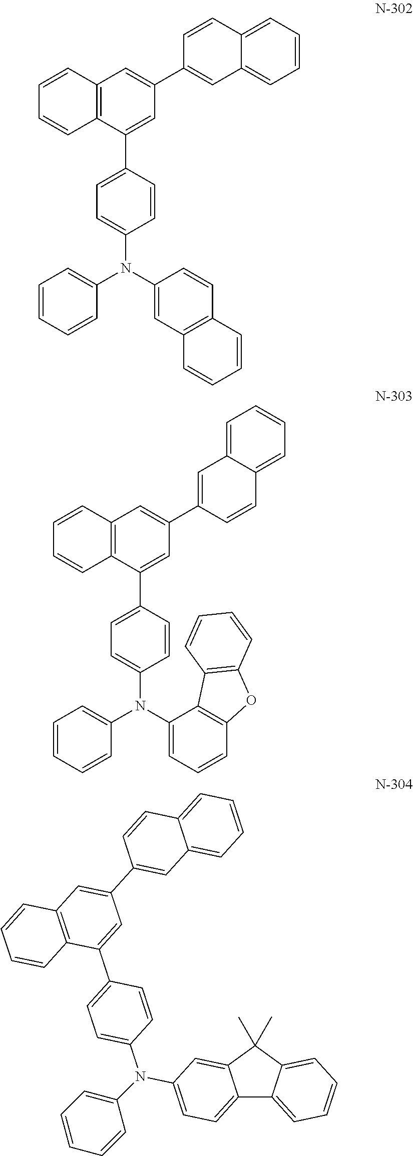

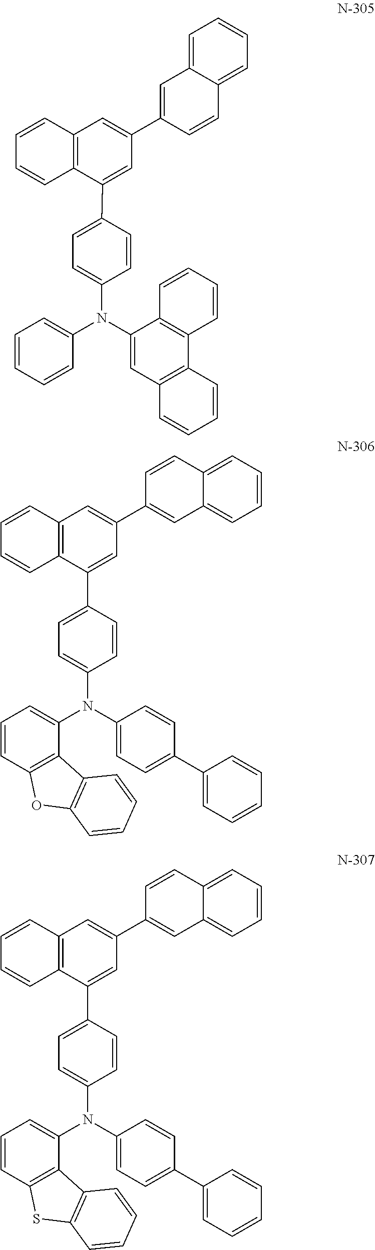

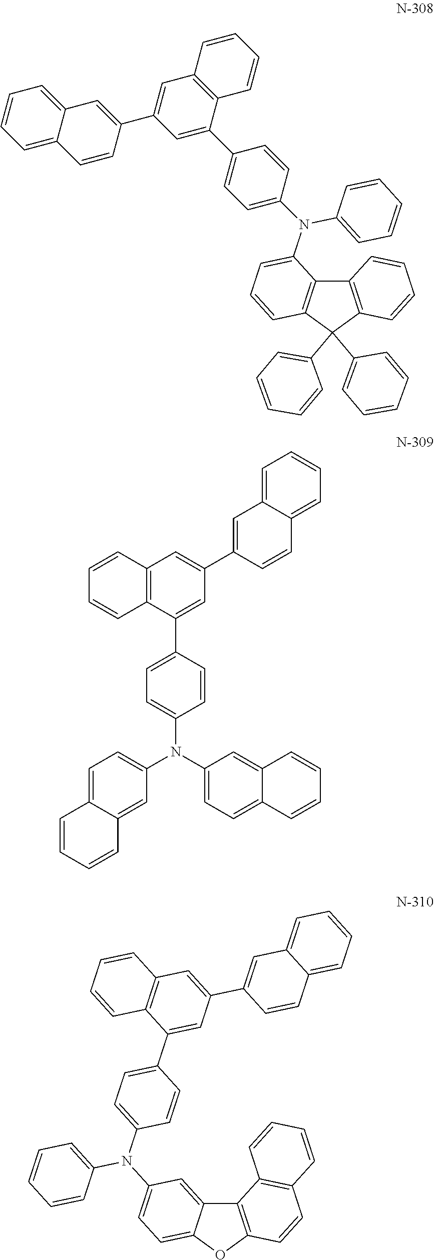

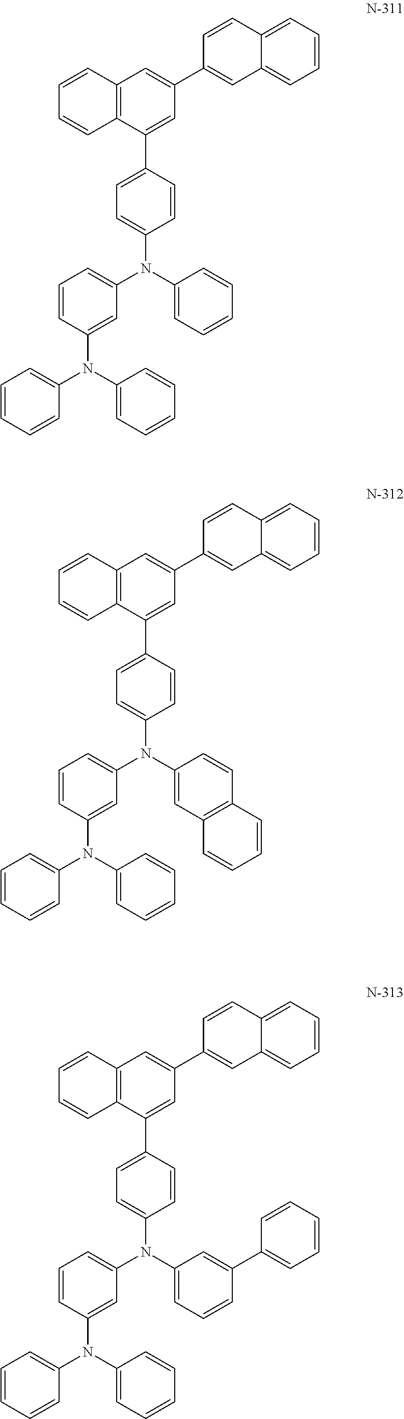 [SMILES: c1ccc(N(c2ccccc2)c2cccc(N(c3ccccc3)c3ccc(-c4cc(-c5ccc6ccccc6c5)cc5ccccc45)cc3)c2)cc1 ; c1ccc(N(c2ccccc2)c2cccc(N(c3ccc(-c4cc(-c5ccc6ccccc6c5)cc5ccccc45)cc3)c3ccc4ccccc4c3)c2)cc1 ; c1ccc(-c2cccc(N(c3ccc(-c4cc(-c5ccc6ccccc6c5)cc5ccccc45)cc3)c3cccc(N(c4ccccc4)c4ccccc4)c3)c2)cc1]

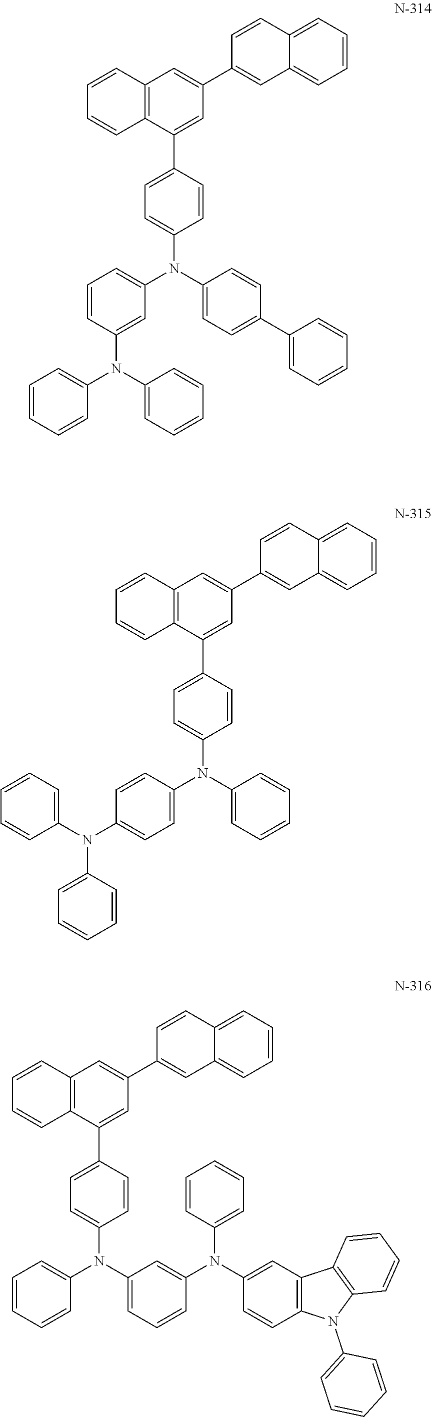 [SMILES: c1ccc(-c2ccc(N(c3ccc(-c4cc(-c5ccc6ccccc6c5)cc5ccccc45)cc3)c3cccc(N(c4ccccc4)c4ccccc4)c3)cc2)cc1 ; c1ccc(N(c2ccccc2)c2ccc(N(c3ccccc3)c3ccc(-c4cc(-c5ccc6ccccc6c5)cc5ccccc45)cc3)cc2)cc1 ; c1ccc(N(c2ccc(-c3cc(-c4ccc5ccccc5c4)cc4ccccc34)cc2)c2cccc(N(c3ccccc3)c3ccc4c(c3)c3ccccc3n4-c3ccccc3)c2)cc1]

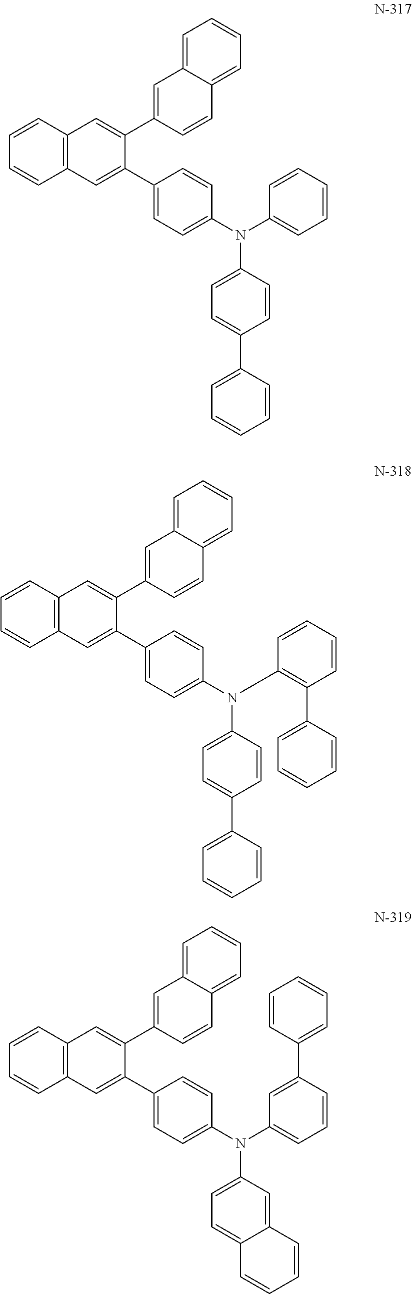

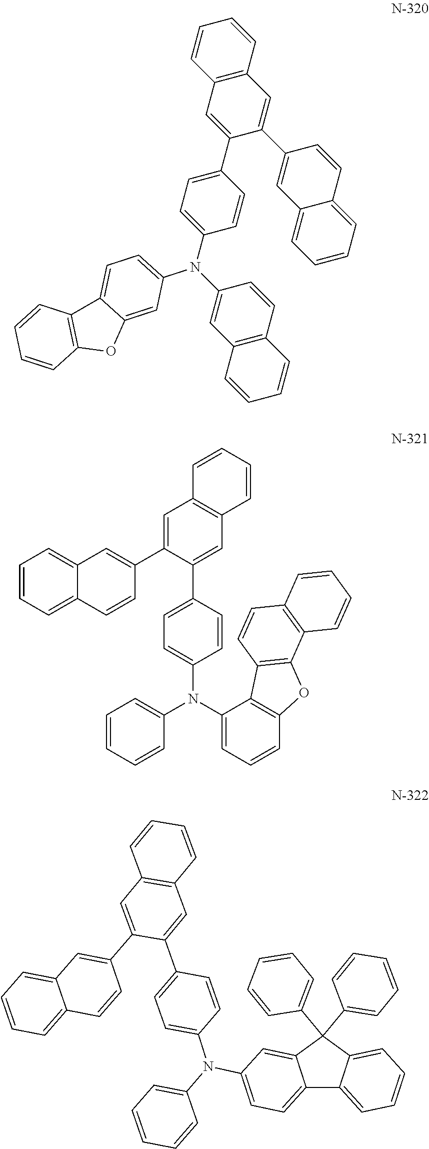 [SMILES: c1ccc2cc(-c3cc4ccccc4cc3-c3ccc(N(c4ccc5ccccc5c4)c4ccc5c(c4)oc4ccccc45)cc3)ccc2c1 ; c1ccc(N(c2ccc(-c3cc4ccccc4cc3-c3ccc4ccccc4c3)cc2)c2cccc3oc4c5ccccc5ccc4c23)cc1 ; c1ccc(N(c2ccc(-c3cc4ccccc4cc3-c3ccc4ccccc4c3)cc2)c2ccc3c(c2)C(c2ccccc2)(c2ccccc2)c2ccccc2-3)cc1]

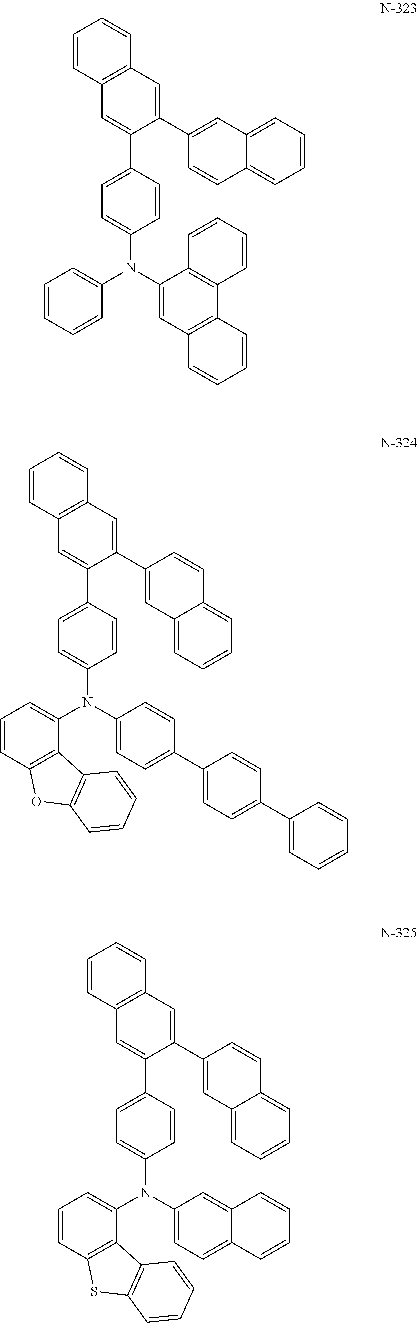 [SMILES: c1ccc(N(c2ccc(-c3cc4ccccc4cc3-c3ccc4ccccc4c3)cc2)c2cc3ccccc3c3ccccc23)cc1 ; c1ccc(-c2ccc(-c3ccc(N(c4ccc(-c5cc6ccccc6cc5-c5ccc6ccccc6c5)cc4)c4cccc5oc6ccccc6c45)cc3)cc2)cc1 ; c1ccc2cc(-c3cc4ccccc4cc3-c3ccc(N(c4ccc5ccccc5c4)c4cccc5sc6ccccc6c45)cc3)ccc2c1]

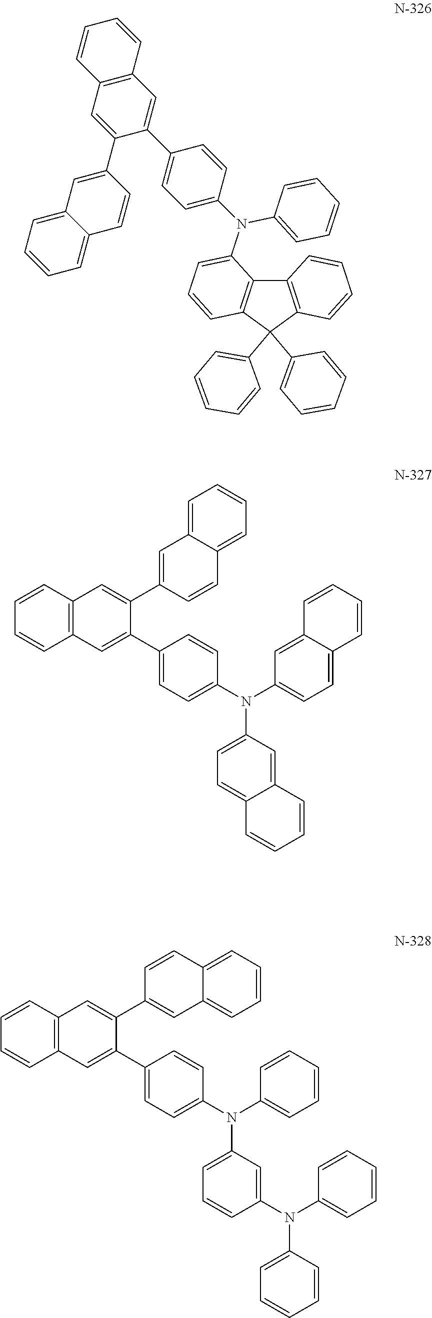 [SMILES: c1ccc(N(c2ccc(-c3cc4ccccc4cc3-c3ccc4ccccc4c3)cc2)c2cccc3c2-c2ccccc2C3(c2ccccc2)c2ccccc2)cc1 ; c1ccc2cc(-c3cc4ccccc4cc3-c3ccc(N(c4ccc5ccccc5c4)c4ccc5ccccc5c4)cc3)ccc2c1 ; c1ccc(N(c2ccccc2)c2cccc(N(c3ccccc3)c3ccc(-c4cc5ccccc5cc4-c4ccc5ccccc5c4)cc3)c2)cc1]

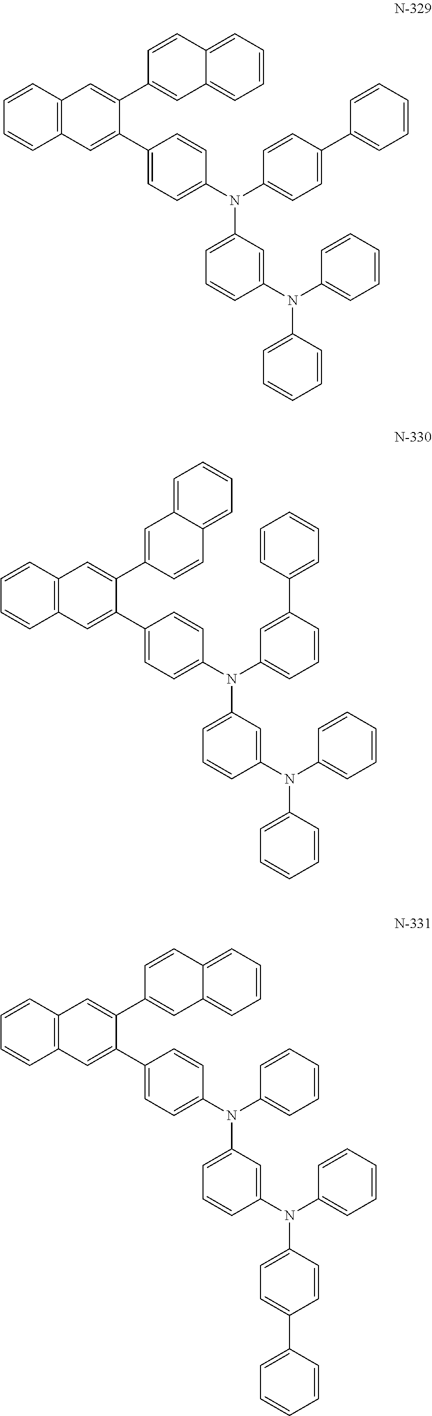 [SMILES: c1ccc(-c2ccc(N(c3ccc(-c4cc5ccccc5cc4-c4ccc5ccccc5c4)cc3)c3cccc(N(c4ccccc4)c4ccccc4)c3)cc2)cc1 ; c1ccc(-c2cccc(N(c3ccc(-c4cc5ccccc5cc4-c4ccc5ccccc5c4)cc3)c3cccc(N(c4ccccc4)c4ccccc4)c3)c2)cc1 ; c1ccc(-c2ccc(N(c3ccccc3)c3cccc(N(c4ccccc4)c4ccc(-c5cc6ccccc6cc5-c5ccc6ccccc6c5)cc4)c3)cc2)cc1]

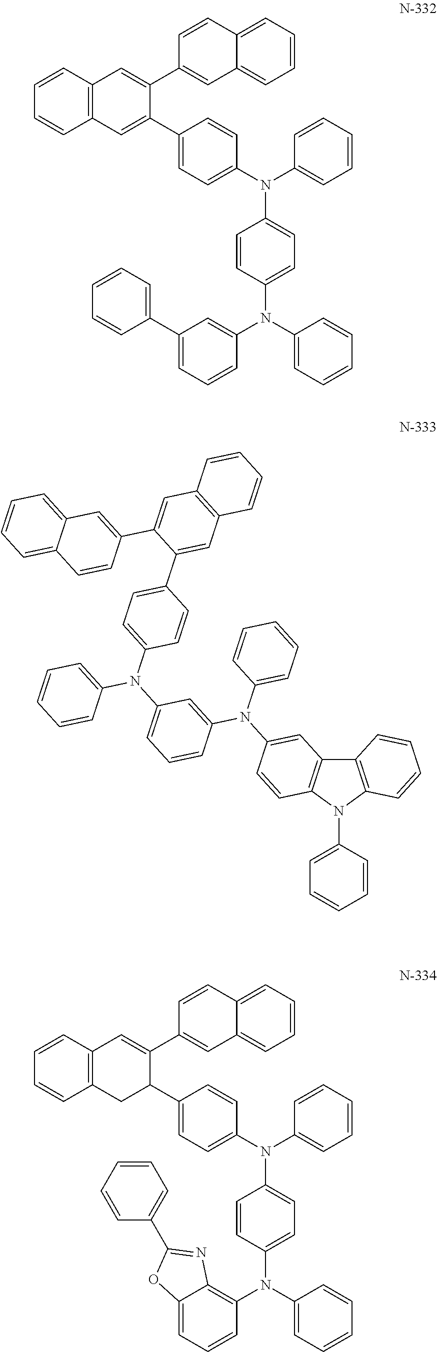 [SMILES: c1ccc(-c2cccc(N(c3ccccc3)c3ccc(N(c4ccccc4)c4ccc(-c5cc6ccccc6cc5-c5ccc6ccccc6c5)cc4)cc3)c2)cc1 ; c1ccc(N(c2ccc(-c3cc4ccccc4cc3-c3ccc4ccccc4c3)cc2)c2cccc(N(c3ccccc3)c3ccc4c(c3)c3ccccc3n4-c3ccccc3)c2)cc1 ; C1=C(c2ccc3ccccc3c2)C(c2ccc(N(c3ccccc3)c3ccc(N(c4ccccc4)c4cccc5oc(-c6ccccc6)nc45)cc3)cc2)Cc2ccccc21]

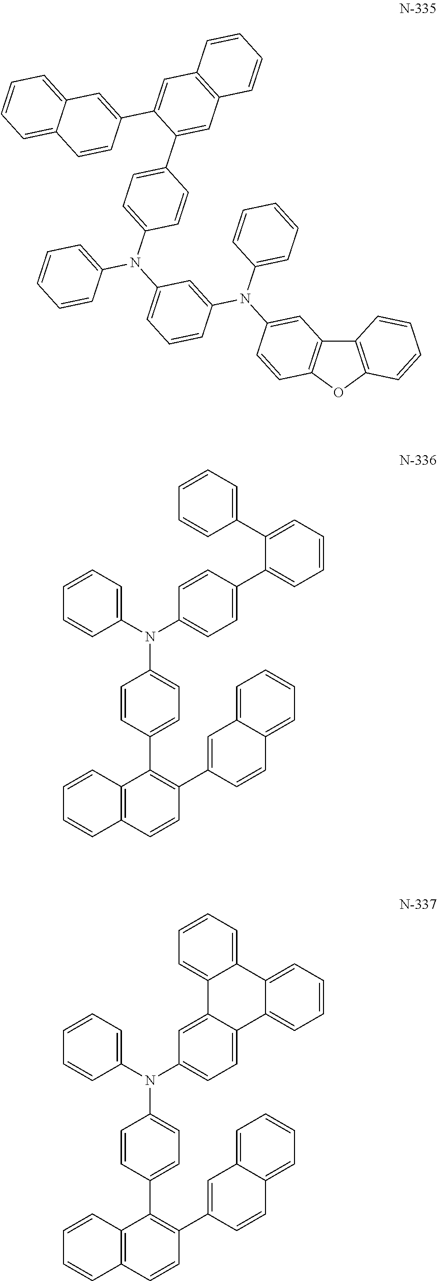 [SMILES: c1ccc(N(c2ccc(-c3cc4ccccc4cc3-c3ccc4ccccc4c3)cc2)c2cccc(N(c3ccccc3)c3ccc4oc5ccccc5c4c3)c2)cc1 ; c1ccc(-c2ccccc2-c2ccc(N(c3ccccc3)c3ccc(-c4c(-c5ccc6ccccc6c5)ccc5ccccc45)cc3)cc2)cc1 ; c1ccc(N(c2ccc(-c3c(-c4ccc5ccccc5c4)ccc4ccccc34)cc2)c2ccc3c4ccccc4c4ccccc4c3c2)cc1]

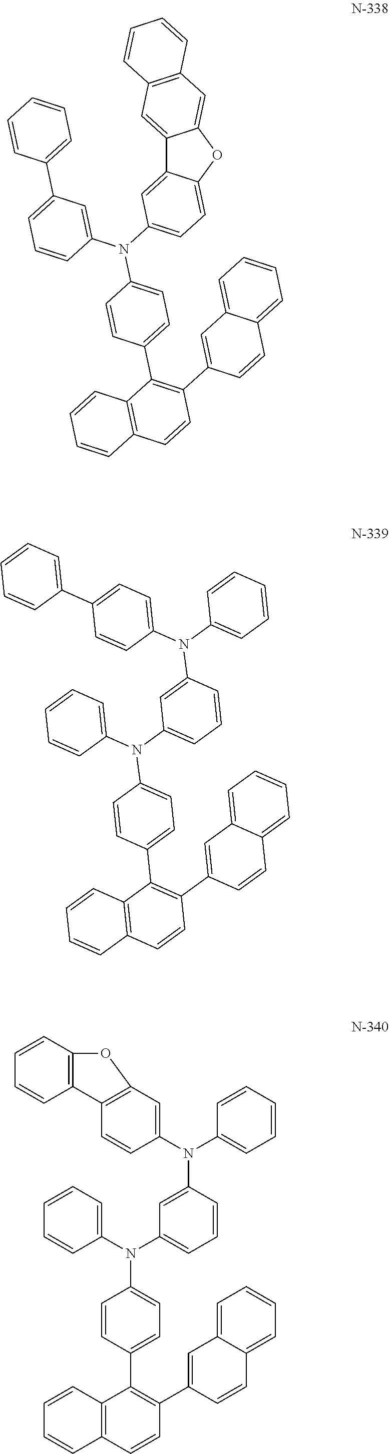 [SMILES: c1ccc(-c2cccc(N(c3ccc(-c4c(-c5ccc6ccccc6c5)ccc5ccccc45)cc3)c3ccc4oc5cc6ccccc6cc5c4c3)c2)cc1 ; c1ccc(-c2ccc(N(c3ccccc3)c3cccc(N(c4ccccc4)c4ccc(-c5c(-c6ccc7ccccc7c6)ccc6ccccc56)cc4)c3)cc2)cc1 ; c1ccc(N(c2ccc(-c3c(-c4ccc5ccccc5c4)ccc4ccccc34)cc2)c2cccc(N(c3ccccc3)c3ccc4c(c3)oc3ccccc34)c2)cc1]

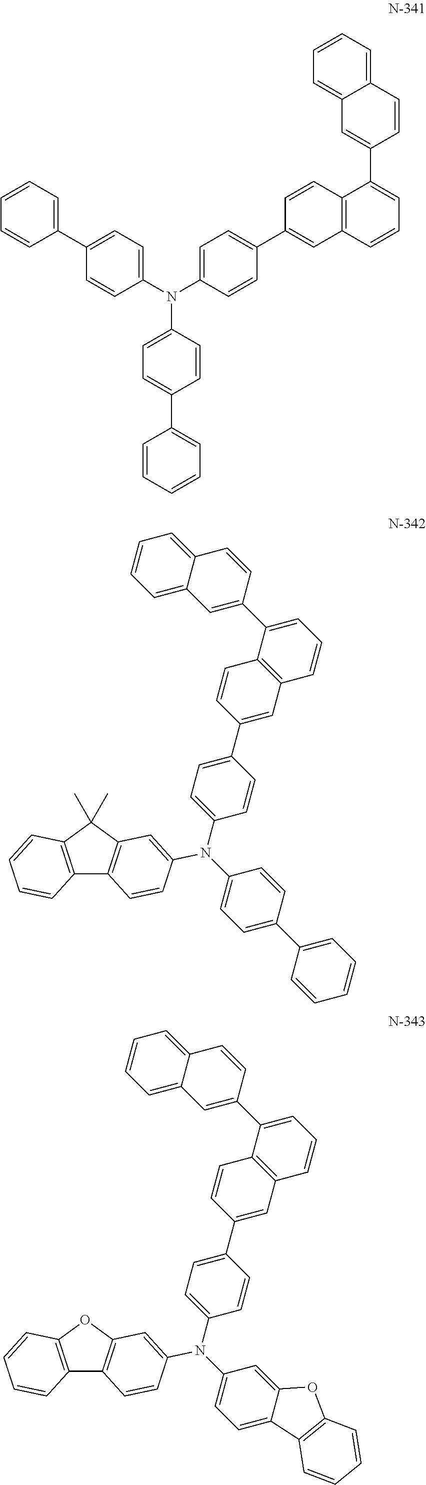

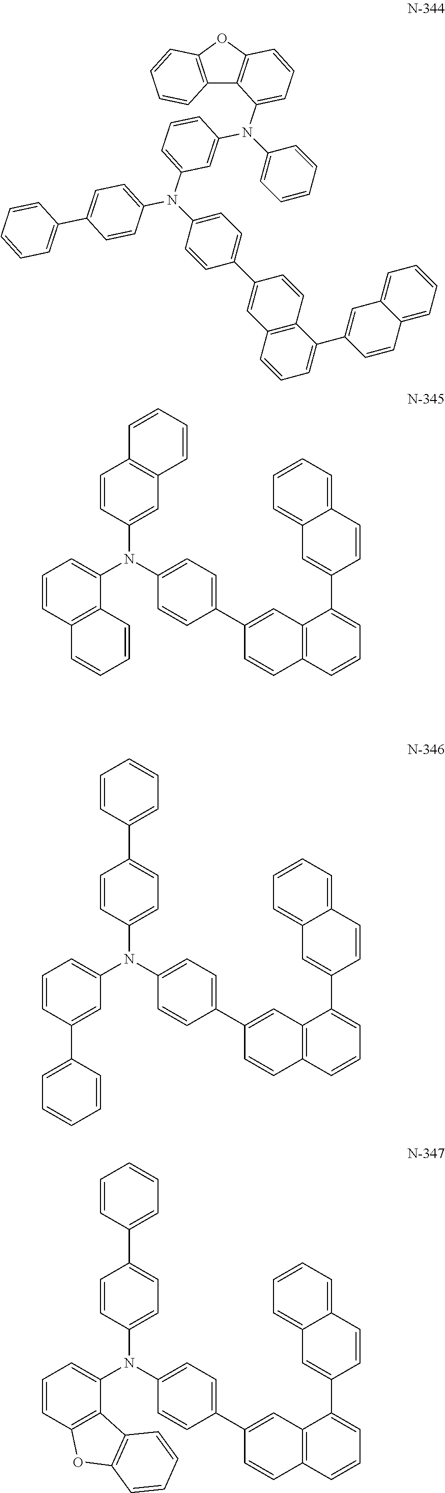 [SMILES: c1ccc(-c2ccc(N(c3ccc(-c4ccc5c(-c6ccc7ccccc7c6)cccc5c4)cc3)c3cccc(N(c4ccccc4)c4cccc5oc6ccccc6c45)c3)cc2)cc1 ; c1ccc2cc(-c3cccc4ccc(-c5ccc(N(c6ccc7ccccc7c6)c6cccc7ccccc67)cc5)cc34)ccc2c1 ; c1ccc(-c2ccc(N(c3ccc(-c4ccc5cccc(-c6ccc7ccccc7c6)c5c4)cc3)c3cccc(-c4ccccc4)c3)cc2)cc1 ; c1ccc(-c2ccc(N(c3ccc(-c4ccc5cccc(-c6ccc7ccccc7c6)c5c4)cc3)c3cccc4oc5ccccc5c34)cc2)cc1]

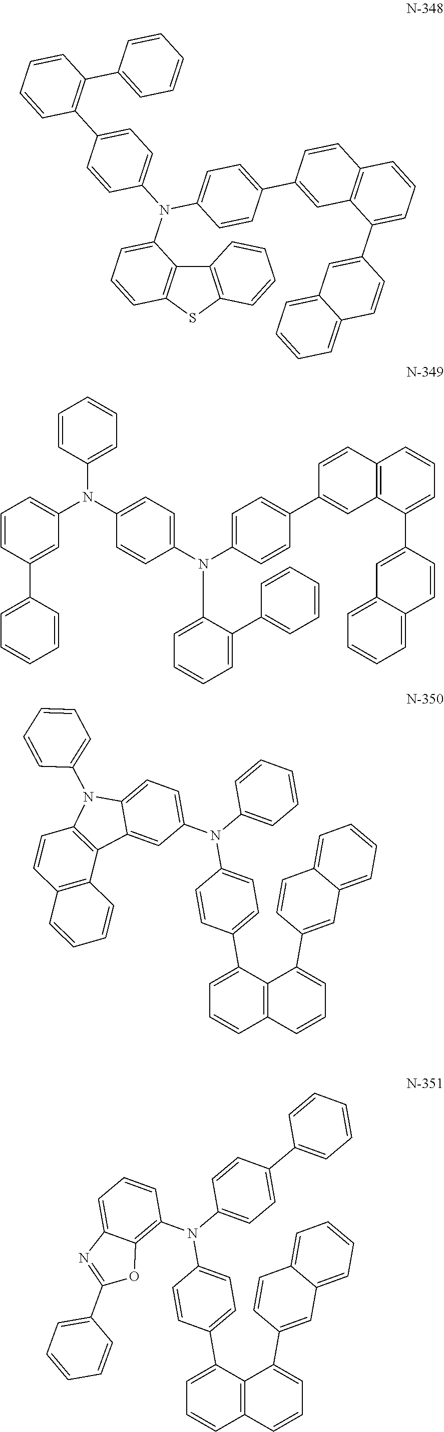

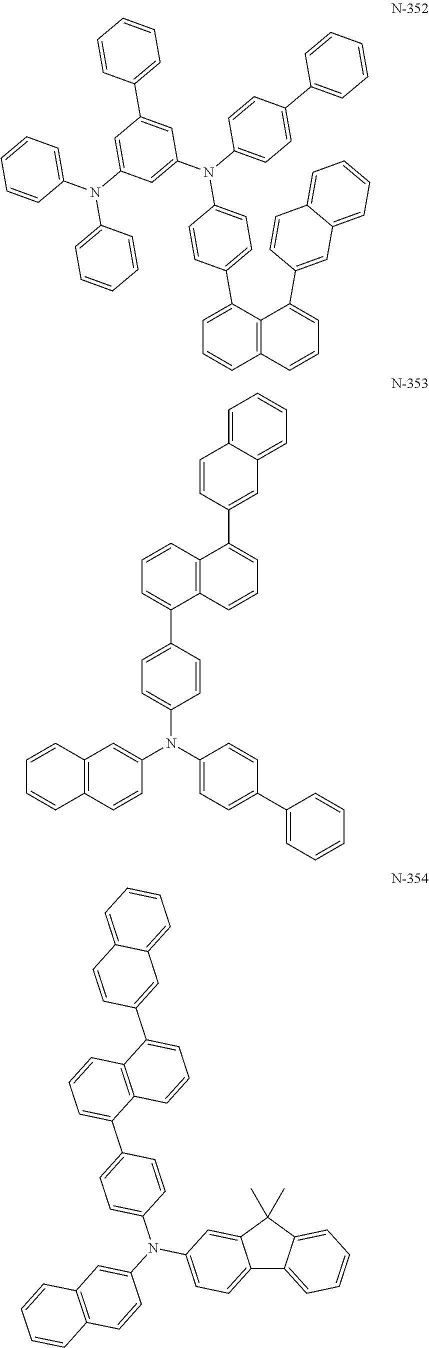

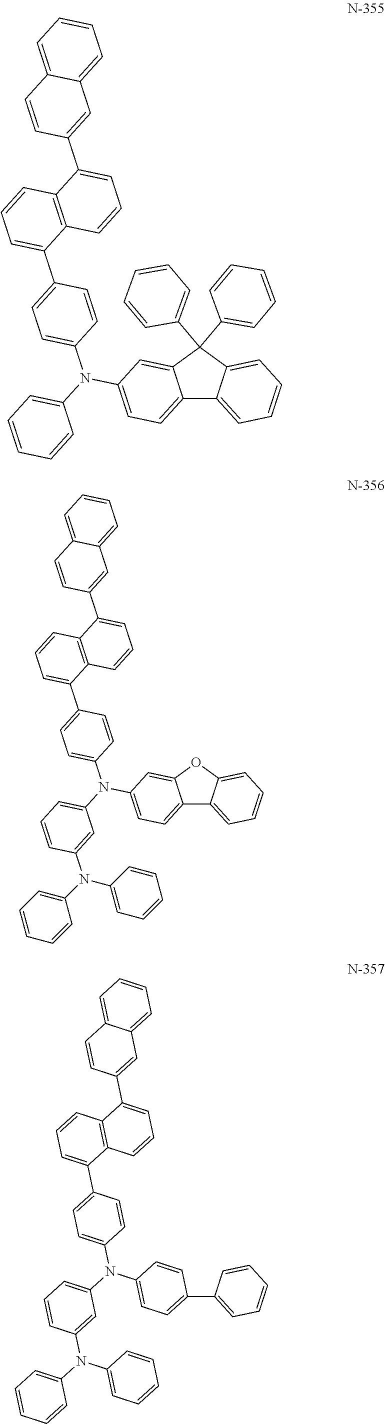 [SMILES: c1ccc(N(c2ccc(-c3cccc4c(-c5ccc6ccccc6c5)cccc34)cc2)c2ccc3c(c2)C(c2ccccc2)(c2ccccc2)c2ccccc2-3)cc1 ; c1ccc(N(c2ccccc2)c2cccc(N(c3ccc(-c4cccc5c(-c6ccc7ccccc7c6)cccc45)cc3)c3ccc4c(c3)oc3ccccc34)c2)cc1 ; c1ccc(-c2ccc(N(c3ccc(-c4cccc5c(-c6ccc7ccccc7c6)cccc45)cc3)c3cccc(N(c4ccccc4)c4ccccc4)c3)cc2)cc1]

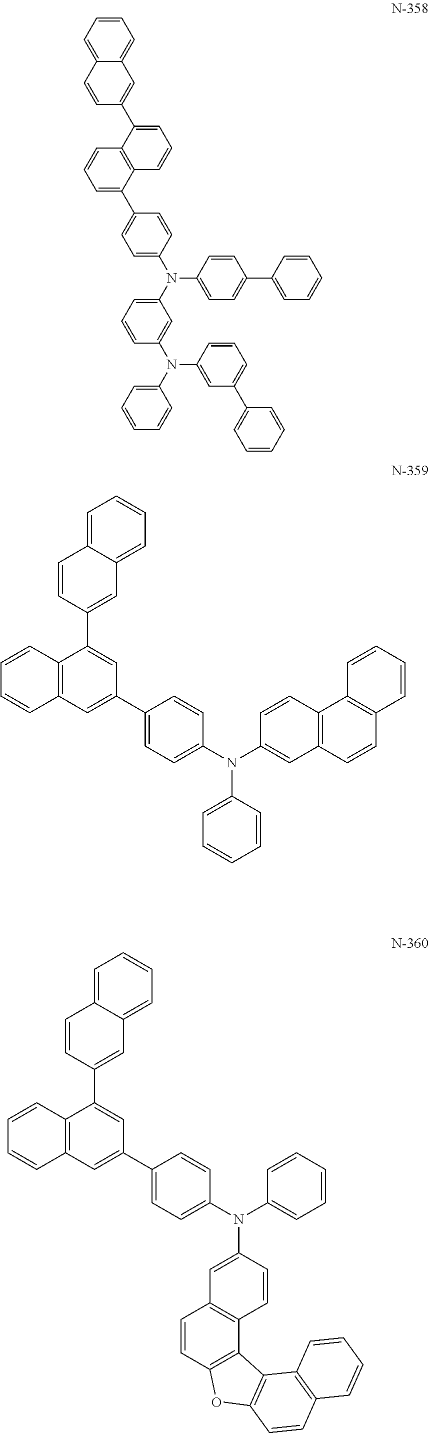 [SMILES: c1ccc(-c2ccc(N(c3ccc(-c4cccc5c(-c6ccc7ccccc7c6)cccc45)cc3)c3cccc(N(c4ccccc4)c4cccc(-c5ccccc5)c4)c3)cc2)cc1 ; c1ccc(N(c2ccc(-c3cc(-c4ccc5ccccc5c4)c4ccccc4c3)cc2)c2ccc3c(ccc4ccccc43)c2)cc1 ; c1ccc(N(c2ccc(-c3cc(-c4ccc5ccccc5c4)c4ccccc4c3)cc2)c2ccc3c(ccc4oc5ccc6ccccc6c5c43)c2)cc1]

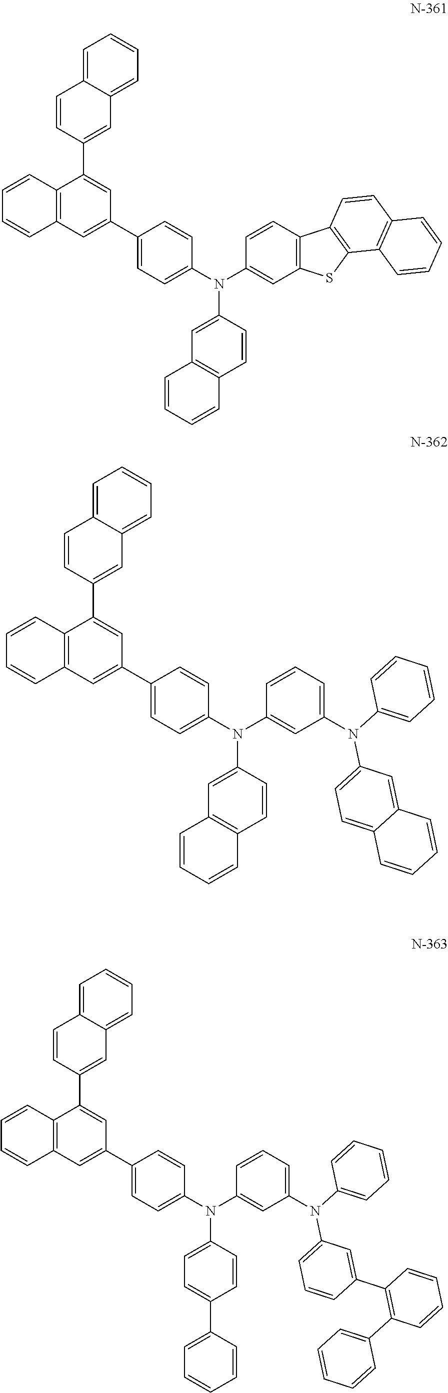 [SMILES: c1ccc2cc(-c3cc(-c4ccc(N(c5ccc6ccccc6c5)c5ccc6c(c5)sc5c7ccccc7ccc65)cc4)cc4ccccc34)ccc2c1 ; c1ccc(N(c2cccc(N(c3ccc(-c4cc(-c5ccc6ccccc6c5)c5ccccc5c4)cc3)c3ccc4ccccc4c3)c2)c2ccc3ccccc3c2)cc1 ; c1ccc(-c2ccc(N(c3ccc(-c4cc(-c5ccc6ccccc6c5)c5ccccc5c4)cc3)c3cccc(N(c4ccccc4)c4cccc(-c5ccccc5-c5ccccc5)c4)c3)cc2)cc1]

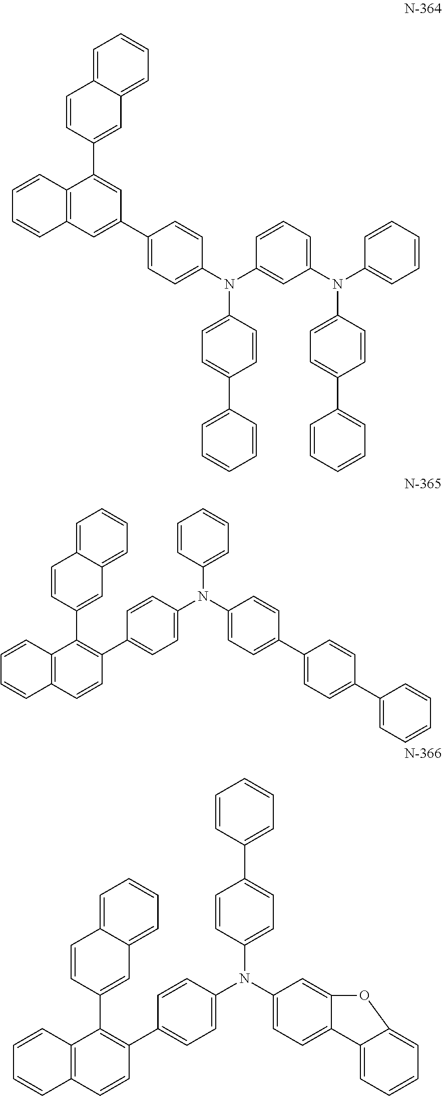 [SMILES: c1ccc(-c2ccc(N(c3ccccc3)c3cccc(N(c4ccc(-c5ccccc5)cc4)c4ccc(-c5cc(-c6ccc7ccccc7c6)c6ccccc6c5)cc4)c3)cc2)cc1 ; c1ccc(-c2ccc(-c3ccc(N(c4ccccc4)c4ccc(-c5ccc6ccccc6c5-c5ccc6ccccc6c5)cc4)cc3)cc2)cc1 ; c1ccc(-c2ccc(N(c3ccc(-c4ccc5ccccc5c4-c4ccc5ccccc5c4)cc3)c3ccc4c(c3)oc3ccccc34)cc2)cc1]

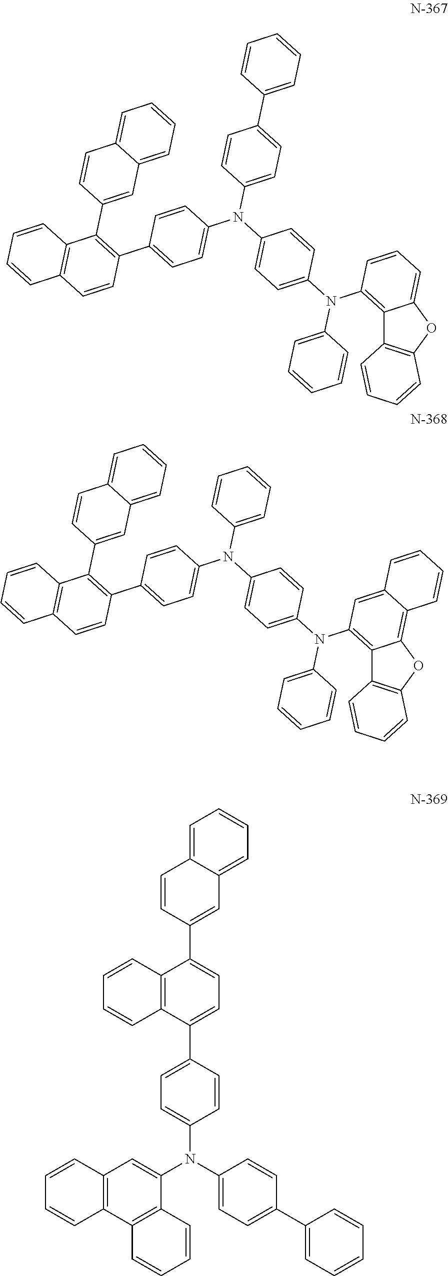 [SMILES: c1ccc(-c2ccc(N(c3ccc(-c4ccc5ccccc5c4-c4ccc5ccccc5c4)cc3)c3ccc(N(c4ccccc4)c4cccc5oc6ccccc6c45)cc3)cc2)cc1 ; c1ccc(N(c2ccc(-c3ccc4ccccc4c3-c3ccc4ccccc4c3)cc2)c2ccc(N(c3ccccc3)c3cc4ccccc4c4oc5ccccc5c34)cc2)cc1 ; c1ccc(-c2ccc(N(c3ccc(-c4ccc(-c5ccc6ccccc6c5)c5ccccc45)cc3)c3cc4ccccc4c4ccccc34)cc2)cc1]

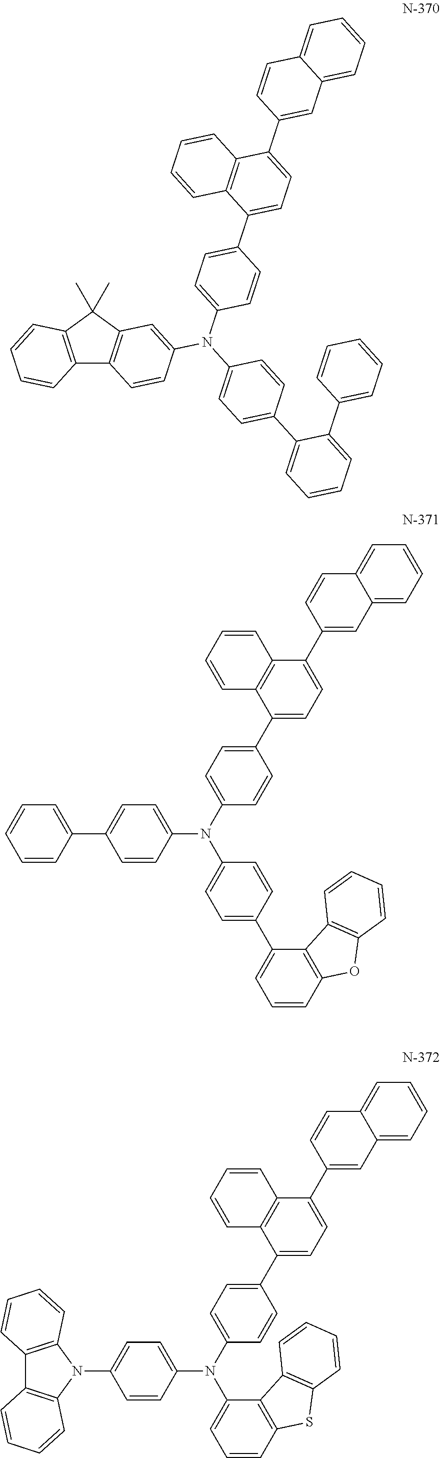 [SMILES: CC1(C)c2ccccc2-c2ccc(N(c3ccc(-c4ccccc4-c4ccccc4)cc3)c3ccc(-c4ccc(-c5ccc6ccccc6c5)c5ccccc45)cc3)cc21 ; c1ccc(-c2ccc(N(c3ccc(-c4ccc(-c5ccc6ccccc6c5)c5ccccc45)cc3)c3ccc(-c4cccc5oc6ccccc6c45)cc3)cc2)cc1 ; c1ccc2cc(-c3ccc(-c4ccc(N(c5ccc(-n6c7ccccc7c7ccccc76)cc5)c5cccc6sc7ccccc7c56)cc4)c4ccccc34)ccc2c1]

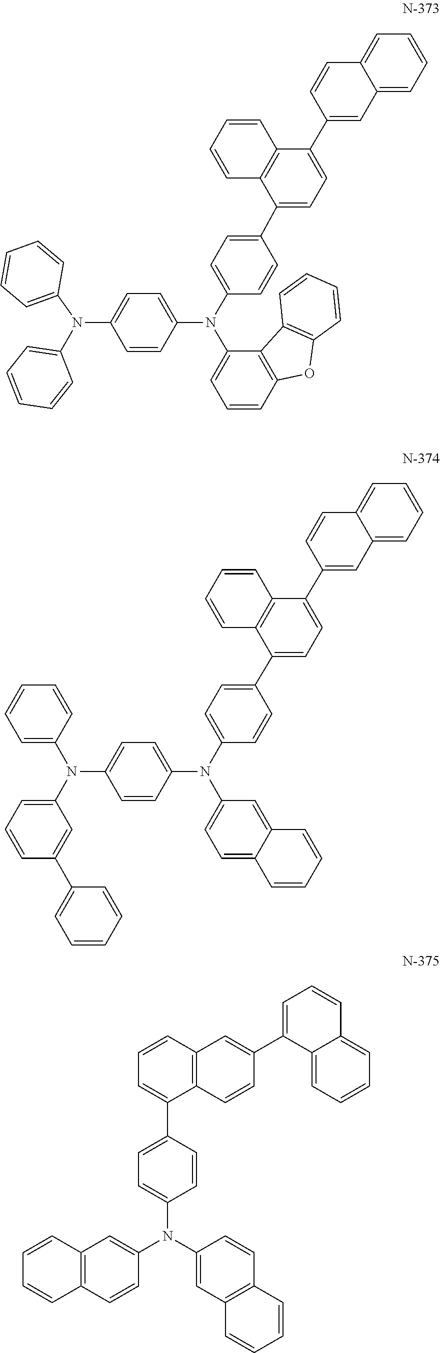 [SMILES: c1ccc(N(c2ccccc2)c2ccc(N(c3ccc(-c4ccc(-c5ccc6ccccc6c5)c5ccccc45)cc3)c3cccc4oc5ccccc5c34)cc2)cc1 ; c1ccc(-c2cccc(N(c3ccccc3)c3ccc(N(c4ccc(-c5ccc(-c6ccc7ccccc7c6)c6ccccc56)cc4)c4ccc5ccccc5c4)cc3)c2)cc1 ; c1ccc2cc(N(c3ccc(-c4cccc5cc(-c6cccc7ccccc67)ccc45)cc3)c3ccc4ccccc4c3)ccc2c1]

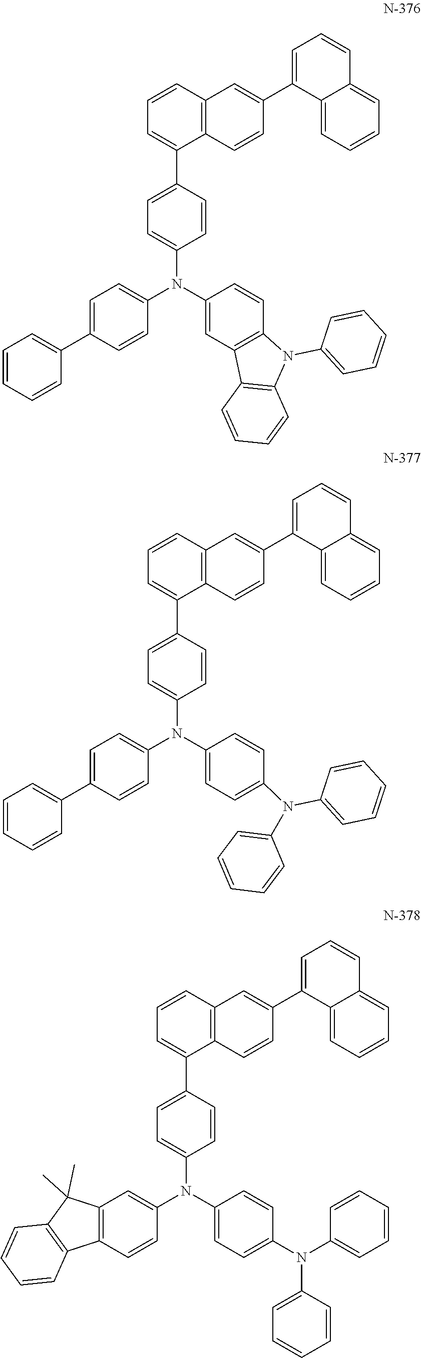 [SMILES: c1ccc(-c2ccc(N(c3ccc(-c4cccc5cc(-c6cccc7ccccc67)ccc45)cc3)c3ccc4c(c3)c3ccccc3n4-c3ccccc3)cc2)cc1 ; c1ccc(-c2ccc(N(c3ccc(-c4cccc5cc(-c6cccc7ccccc67)ccc45)cc3)c3ccc(N(c4ccccc4)c4ccccc4)cc3)cc2)cc1 ; CC1(C)c2ccccc2-c2ccc(N(c3ccc(-c4cccc5cc(-c6cccc7ccccc67)ccc45)cc3)c3ccc(N(c4ccccc4)c4ccccc4)cc3)cc21]

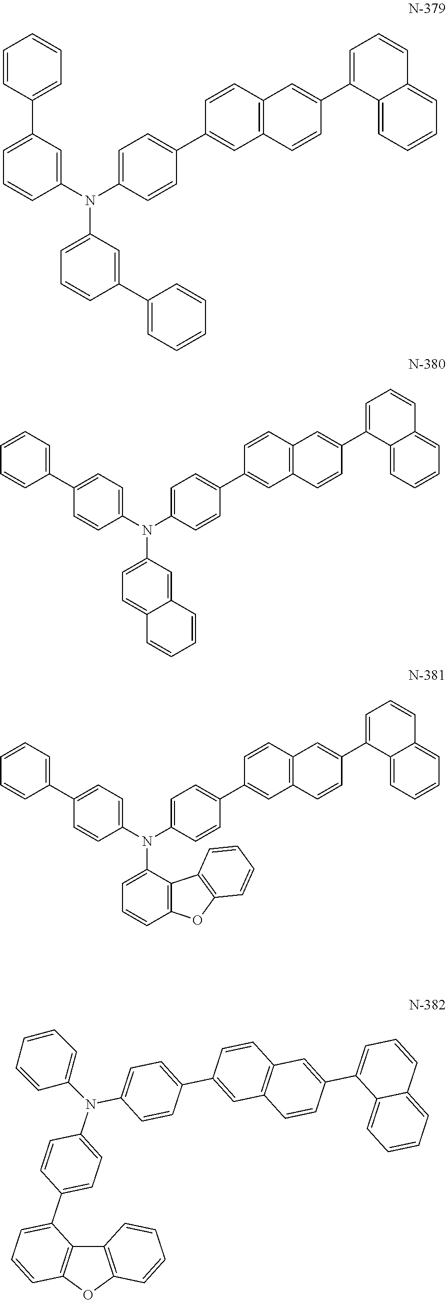 [SMILES: c1ccc(-c2cccc(N(c3ccc(-c4ccc5cc(-c6cccc7ccccc67)ccc5c4)cc3)c3cccc(-c4ccccc4)c3)c2)cc1 ; c1ccc(-c2ccc(N(c3ccc(-c4ccc5cc(-c6cccc7ccccc67)ccc5c4)cc3)c3ccc4ccccc4c3)cc2)cc1 ; c1ccc(-c2ccc(N(c3ccc(-c4ccc5cc(-c6cccc7ccccc67)ccc5c4)cc3)c3cccc4oc5ccccc5c34)cc2)cc1 ; c1ccc(N(c2ccc(-c3ccc4cc(-c5cccc6ccccc56)ccc4c3)cc2)c2ccc(-c3cccc4oc5ccccc5c34)cc2)cc1]

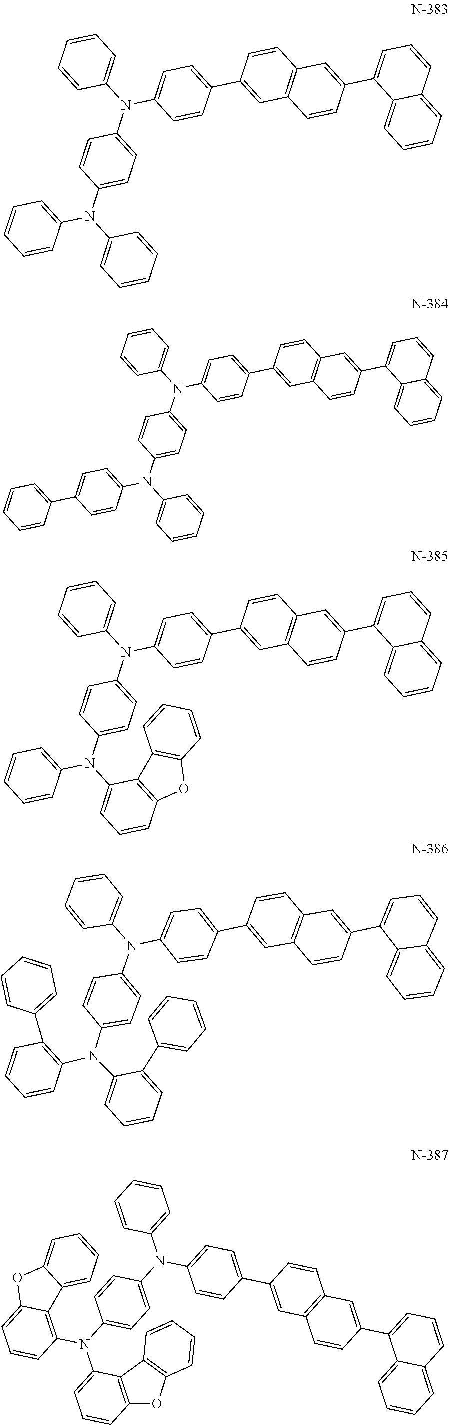 [SMILES: c1ccc(N(c2ccccc2)c2ccc(N(c3ccccc3)c3ccc(-c4ccc5cc(-c6cccc7ccccc67)ccc5c4)cc3)cc2)cc1 ; c1ccc(-c2ccc(N(c3ccccc3)c3ccc(N(c4ccccc4)c4ccc(-c5ccc6cc(-c7cccc8ccccc78)ccc6c5)cc4)cc3)cc2)cc1 ; c1ccc(N(c2ccc(-c3ccc4cc(-c5cccc6ccccc56)ccc4c3)cc2)c2ccc(N(c3ccccc3)c3cccc4oc5ccccc5c34)cc2)cc1 ; c1ccc(-c2ccccc2N(c2ccc(N(c3ccccc3)c3ccc(-c4ccc5cc(-c6cccc7ccccc67)ccc5c4)cc3)cc2)c2ccccc2-c2ccccc2)cc1 ; c1ccc(N(c2ccc(-c3ccc4cc(-c5cccc6ccccc56)ccc4c3)cc2)c2ccc(N(c3cccc4oc5ccccc5c34)c3cccc4oc5ccccc5c34)cc2)cc1]

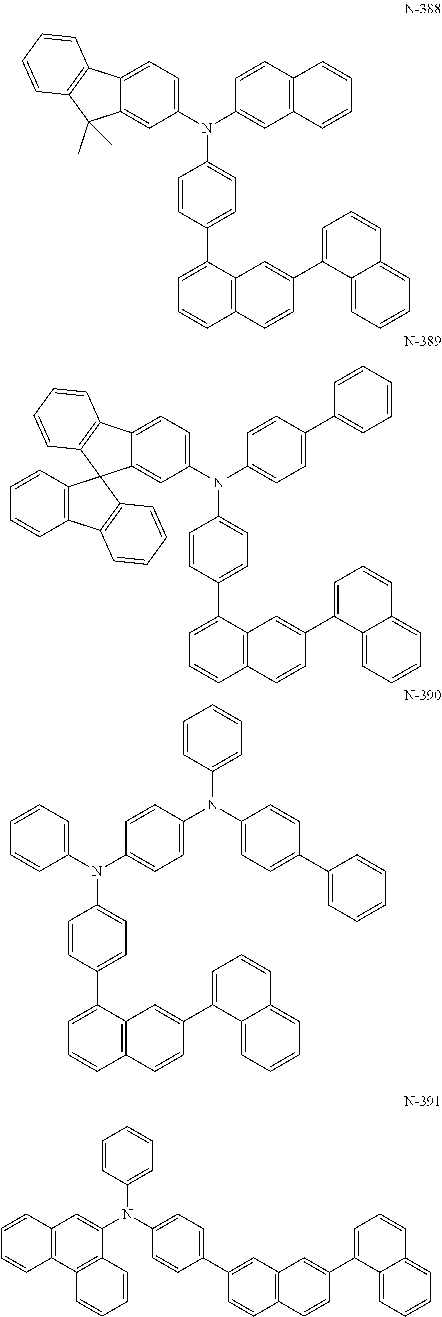

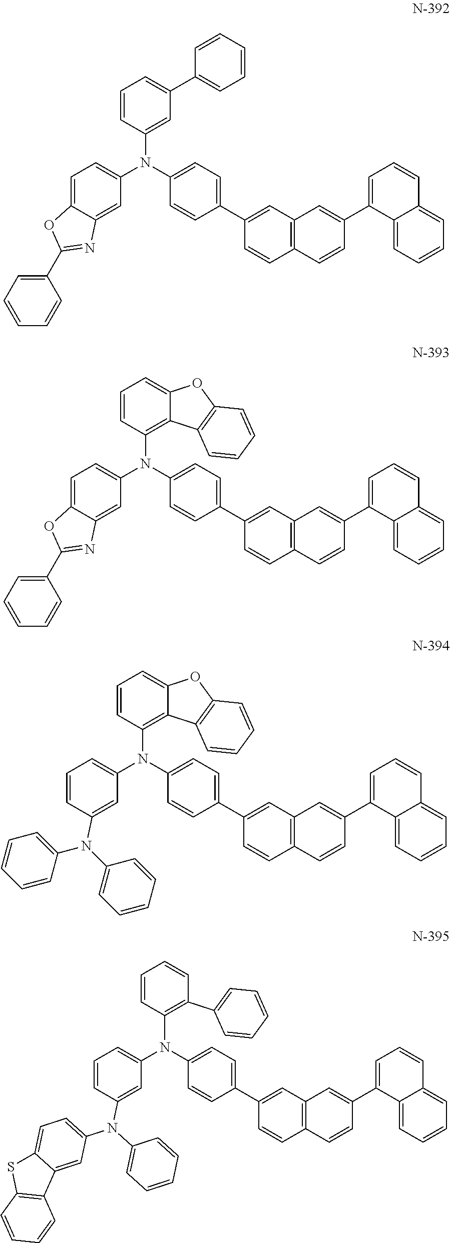 [SMILES: c1ccc(-c2cccc(N(c3ccc(-c4ccc5ccc(-c6cccc7ccccc67)cc5c4)cc3)c3ccc4oc(-c5ccccc5)nc4c3)c2)cc1 ; c1ccc(-c2nc3cc(N(c4ccc(-c5ccc6ccc(-c7cccc8ccccc78)cc6c5)cc4)c4cccc5oc6ccccc6c45)ccc3o2)cc1 ; c1ccc(N(c2ccccc2)c2cccc(N(c3ccc(-c4ccc5ccc(-c6cccc7ccccc67)cc5c4)cc3)c3cccc4oc5ccccc5c34)c2)cc1 ; c1ccc(-c2ccccc2N(c2ccc(-c3ccc4ccc(-c5cccc6ccccc56)cc4c3)cc2)c2cccc(N(c3ccccc3)c3ccc4sc5ccccc5c4c3)c2)cc1]

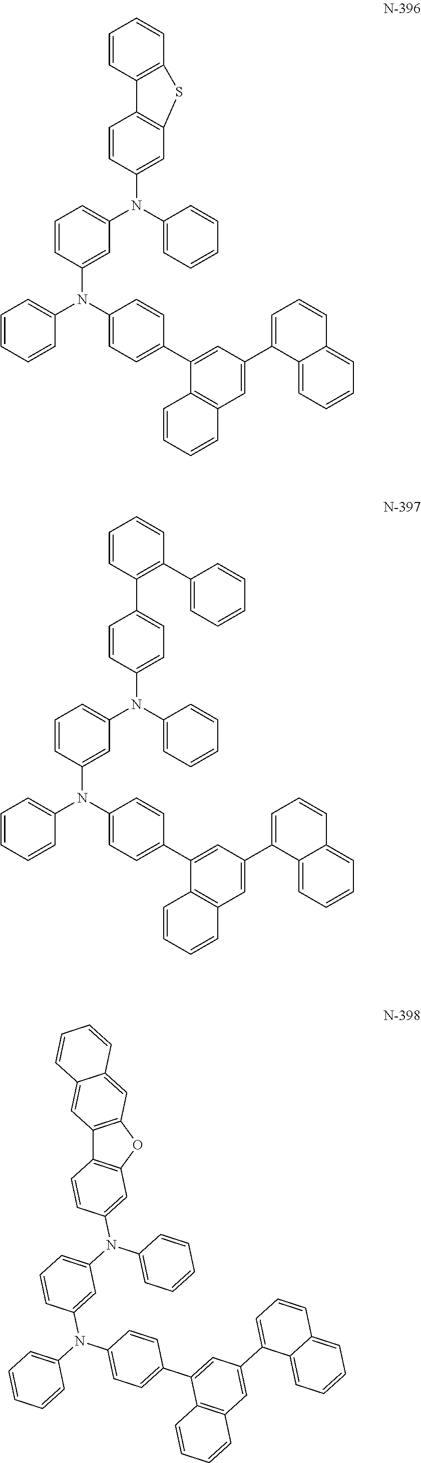 [SMILES: c1ccc(N(c2ccc(-c3cc(-c4cccc5ccccc45)cc4ccccc34)cc2)c2cccc(N(c3ccccc3)c3ccc4c(c3)sc3ccccc34)c2)cc1 ; c1ccc(-c2ccccc2-c2ccc(N(c3ccccc3)c3cccc(N(c4ccccc4)c4ccc(-c5cc(-c6cccc7ccccc67)cc6ccccc56)cc4)c3)cc2)cc1 ; c1ccc(N(c2ccc(-c3cc(-c4cccc5ccccc45)cc4ccccc34)cc2)c2cccc(N(c3ccccc3)c3ccc4c(c3)oc3cc5ccccc5cc34)c2)cc1]

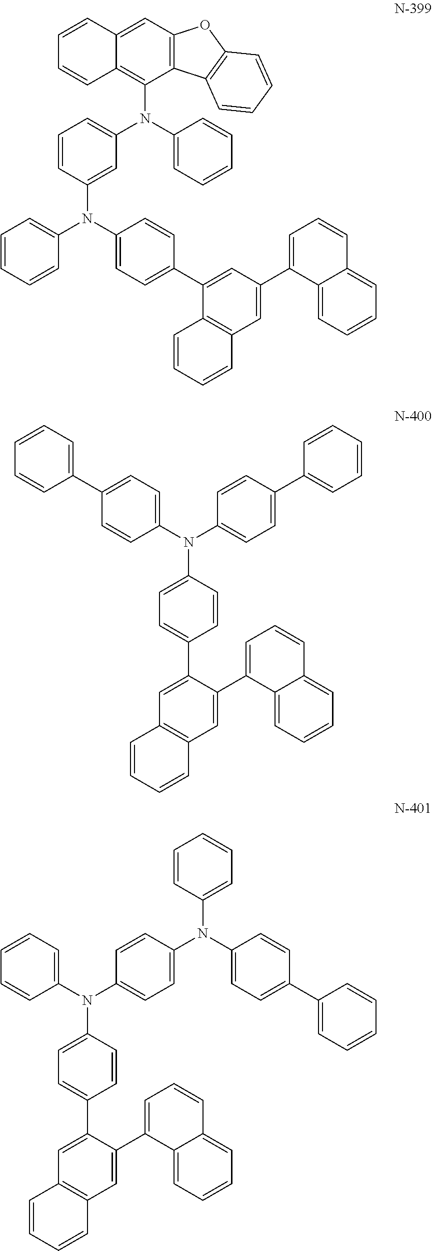 [SMILES: c1ccc(N(c2ccc(-c3cc(-c4cccc5ccccc45)cc4ccccc34)cc2)c2cccc(N(c3ccccc3)c3c4ccccc4cc4oc5ccccc5c34)c2)cc1 ; c1ccc(-c2ccc(N(c3ccc(-c4ccccc4)cc3)c3ccc(-c4cc5ccccc5cc4-c4cccc5ccccc45)cc3)cc2)cc1 ; c1ccc(-c2ccc(N(c3ccccc3)c3ccc(N(c4ccccc4)c4ccc(-c5cc6ccccc6cc5-c5cccc6ccccc56)cc4)cc3)cc2)cc1]

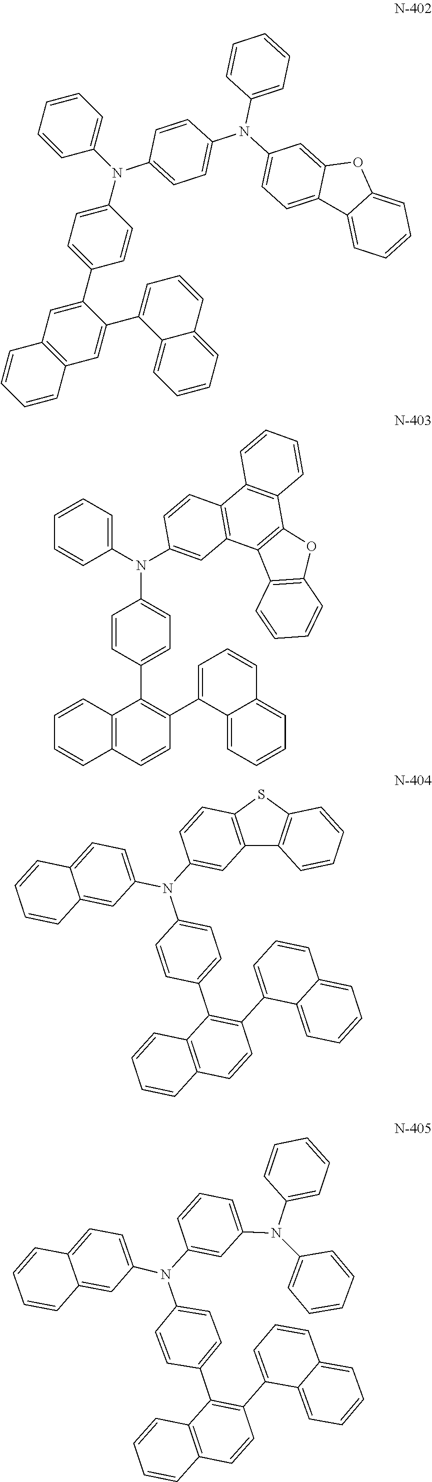

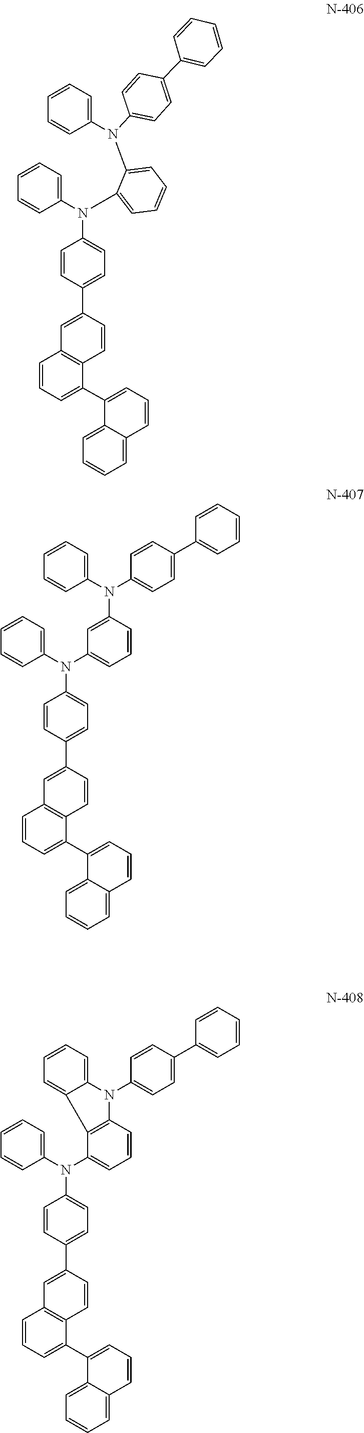 [SMILES: c1ccc(-c2ccc(N(c3ccccc3)c3ccccc3N(c3ccccc3)c3ccc(-c4ccc5c(-c6cccc7ccccc67)cccc5c4)cc3)cc2)cc1 ; c1ccc(-c2ccc(N(c3ccccc3)c3cccc(N(c4ccccc4)c4ccc(-c5ccc6c(-c7cccc8ccccc78)cccc6c5)cc4)c3)cc2)cc1 ; c1ccc(-c2ccc(-n3c4ccccc4c4c(N(c5ccccc5)c5ccc(-c6ccc7c(-c8cccc9ccccc89)cccc7c6)cc5)cccc43)cc2)cc1]

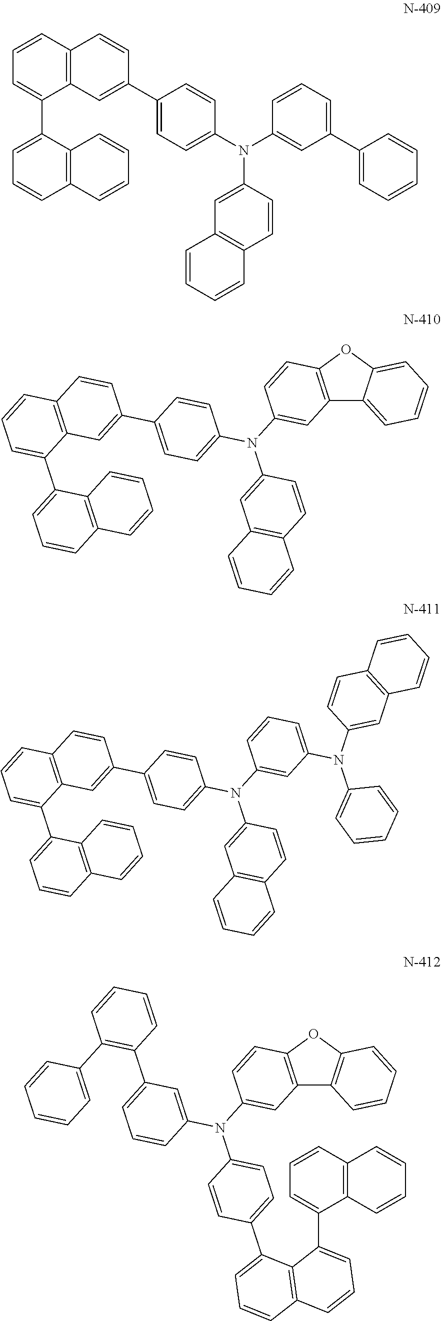 [SMILES: c1ccc(-c2cccc(N(c3ccc(-c4ccc5cccc(-c6cccc7ccccc67)c5c4)cc3)c3ccc4ccccc4c3)c2)cc1 ; c1ccc2cc(N(c3ccc(-c4ccc5cccc(-c6cccc7ccccc67)c5c4)cc3)c3ccc4oc5ccccc5c4c3)ccc2c1 ; c1ccc(N(c2cccc(N(c3ccc(-c4ccc5cccc(-c6cccc7ccccc67)c5c4)cc3)c3ccc4ccccc4c3)c2)c2ccc3ccccc3c2)cc1 ; c1ccc(-c2ccccc2-c2cccc(N(c3ccc(-c4cccc5cccc(-c6cccc7ccccc67)c45)cc3)c3ccc4oc5ccccc5c4c3)c2)cc1]

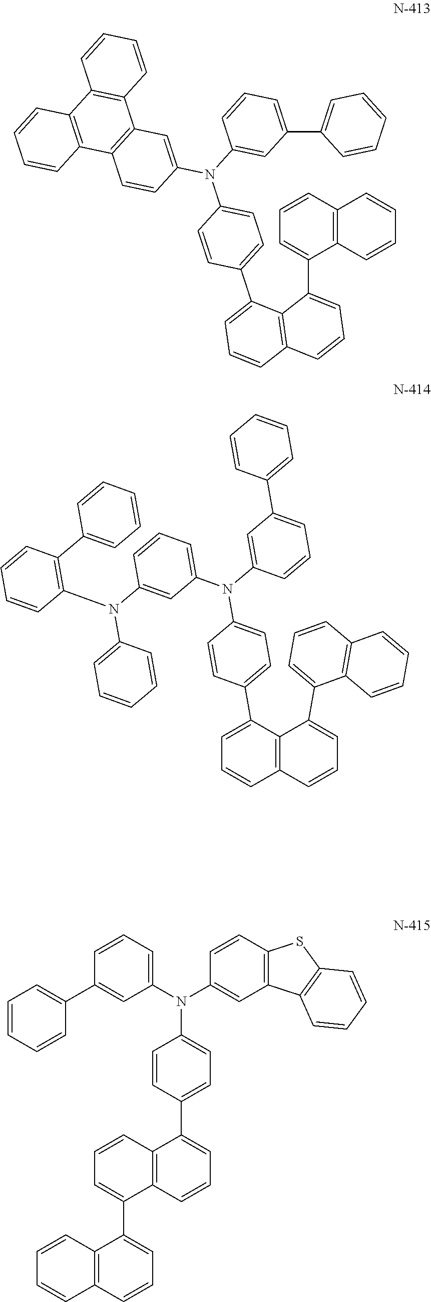 [SMILES: c1ccc(-c2cccc(N(c3ccc(-c4cccc5cccc(-c6cccc7ccccc67)c45)cc3)c3ccc4c5ccccc5c5ccccc5c4c3)c2)cc1 ; c1ccc(-c2cccc(N(c3ccc(-c4cccc5cccc(-c6cccc7ccccc67)c45)cc3)c3cccc(N(c4ccccc4)c4ccccc4-c4ccccc4)c3)c2)cc1 ; c1ccc(-c2cccc(N(c3ccc(-c4cccc5c(-c6cccc7ccccc67)cccc45)cc3)c3ccc4sc5ccccc5c4c3)c2)cc1]

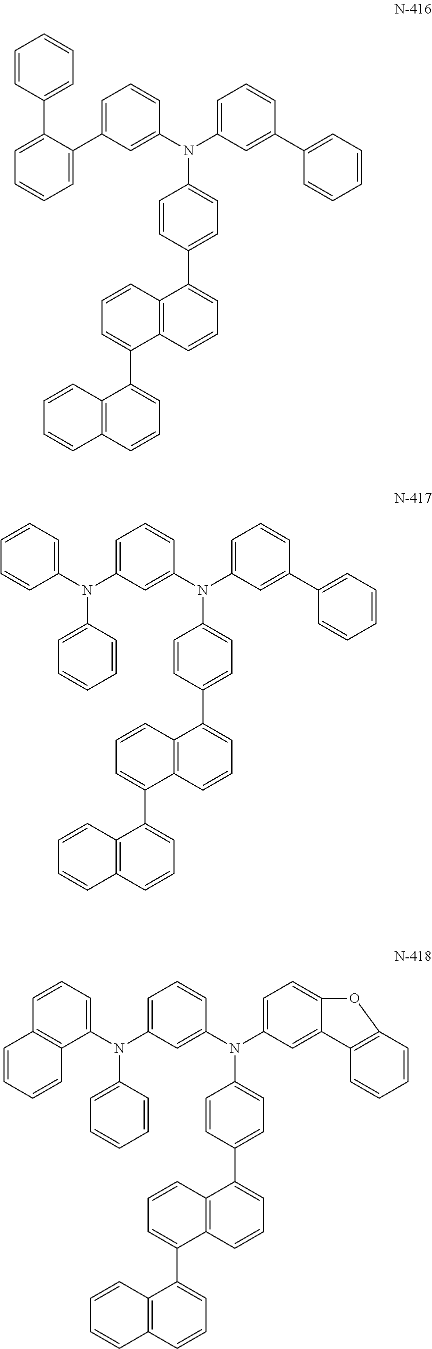 [SMILES: c1ccc(-c2cccc(N(c3ccc(-c4cccc5c(-c6cccc7ccccc67)cccc45)cc3)c3cccc(-c4ccccc4-c4ccccc4)c3)c2)cc1 ; c1ccc(-c2cccc(N(c3ccc(-c4cccc5c(-c6cccc7ccccc67)cccc45)cc3)c3cccc(N(c4ccccc4)c4ccccc4)c3)c2)cc1 ; c1ccc(N(c2cccc(N(c3ccc(-c4cccc5c(-c6cccc7ccccc67)cccc45)cc3)c3ccc4oc5ccccc5c4c3)c2)c2cccc3ccccc23)cc1]

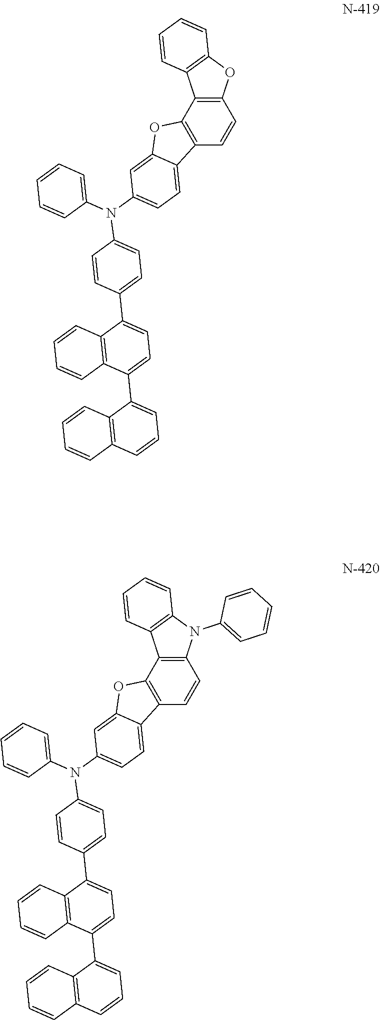 [SMILES: c1ccc(N(c2ccc(-c3ccc(-c4cccc5ccccc45)c4ccccc34)cc2)c2ccc3c(c2)oc2c3ccc3oc4ccccc4c32)cc1 ; c1ccc(N(c2ccc(-c3ccc(-c4cccc5ccccc45)c4ccccc34)cc2)c2ccc3c(c2)oc2c3ccc3c2c2ccccc2n3-c2ccccc2)cc1]

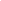

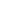

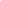

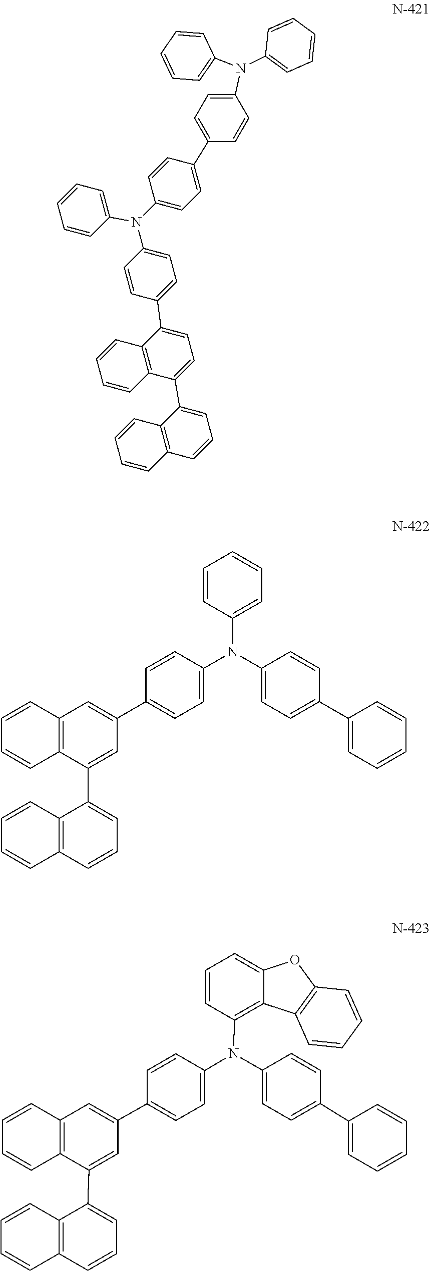 [SMILES: c1ccc(N(c2ccccc2)c2ccc(-c3ccc(N(c4ccccc4)c4ccc(-c5ccc(-c6cccc7ccccc67)c6ccccc56)cc4)cc3)cc2)cc1 ; c1ccc(-c2ccc(N(c3ccccc3)c3ccc(-c4cc(-c5cccc6ccccc56)c5ccccc5c4)cc3)cc2)cc1 ; c1ccc(-c2ccc(N(c3ccc(-c4cc(-c5cccc6ccccc56)c5ccccc5c4)cc3)c3cccc4oc5ccccc5c34)cc2)cc1]

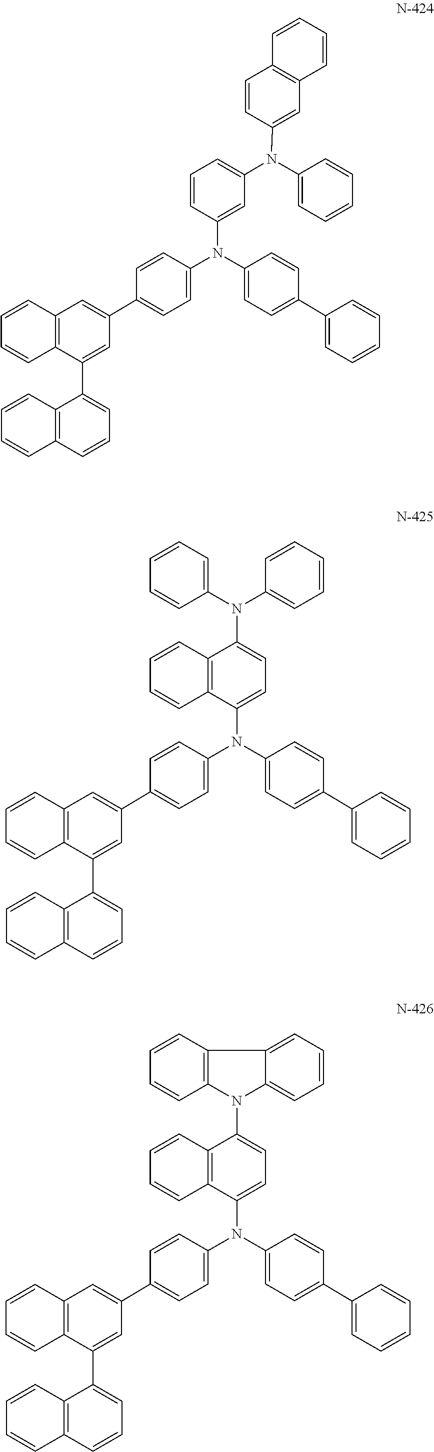 [SMILES: c1ccc(-c2ccc(N(c3ccc(-c4cc(-c5cccc6ccccc56)c5ccccc5c4)cc3)c3cccc(N(c4ccccc4)c4ccc5ccccc5c4)c3)cc2)cc1 ; c1ccc(-c2ccc(N(c3ccc(-c4cc(-c5cccc6ccccc56)c5ccccc5c4)cc3)c3ccc(N(c4ccccc4)c4ccccc4)c4ccccc34)cc2)cc1 ; c1ccc(-c2ccc(N(c3ccc(-c4cc(-c5cccc6ccccc56)c5ccccc5c4)cc3)c3ccc(-n4c5ccccc5c5ccccc54)c4ccccc34)cc2)cc1]

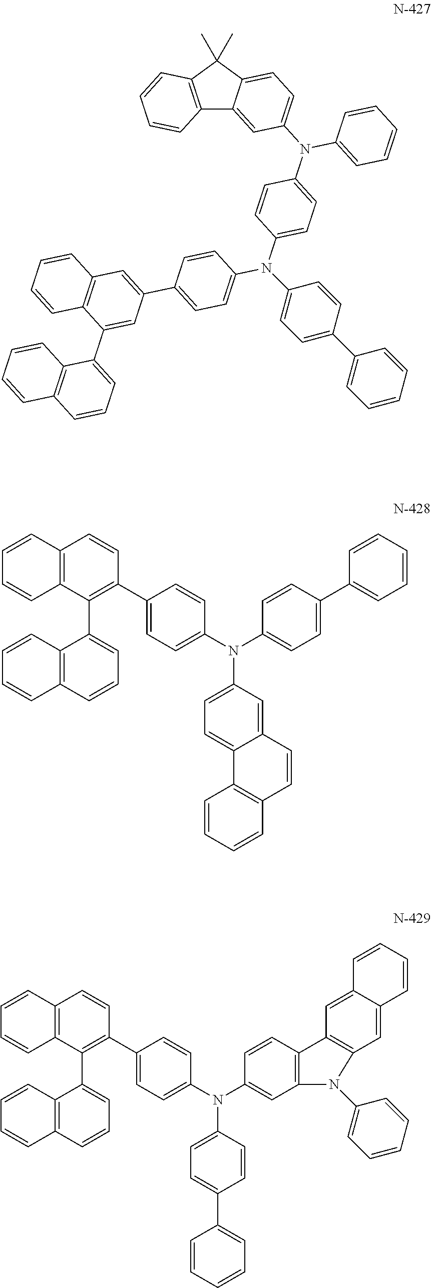 [SMILES: CC1(C)c2ccccc2-c2cc(N(c3ccccc3)c3ccc(N(c4ccc(-c5ccccc5)cc4)c4ccc(-c5cc(-c6cccc7ccccc67)c6ccccc6c5)cc4)cc3)ccc21 ; c1ccc(-c2ccc(N(c3ccc(-c4ccc5ccccc5c4-c4cccc5ccccc45)cc3)c3ccc4c(ccc5ccccc54)c3)cc2)cc1 ; c1ccc(-c2ccc(N(c3ccc(-c4ccc5ccccc5c4-c4cccc5ccccc45)cc3)c3ccc4c5cc6ccccc6cc5n(-c5ccccc5)c4c3)cc2)cc1]

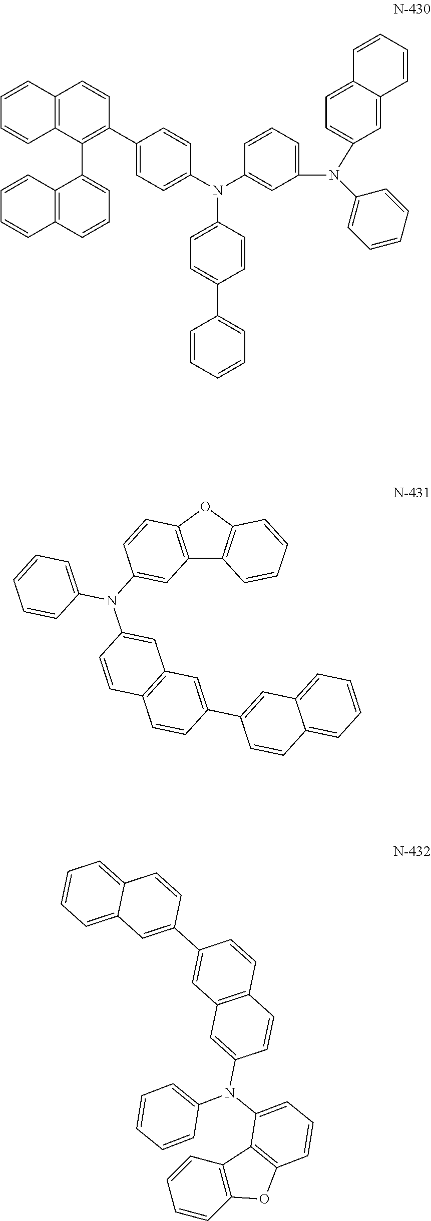 [SMILES: c1ccc(-c2ccc(N(c3ccc(-c4ccc5ccccc5c4-c4cccc5ccccc45)cc3)c3cccc(N(c4ccccc4)c4ccc5ccccc5c4)c3)cc2)cc1 ; c1ccc(N(c2ccc3ccc(-c4ccc5ccccc5c4)cc3c2)c2ccc3oc4ccccc4c3c2)cc1 ; c1ccc(N(c2ccc3ccc(-c4ccc5ccccc5c4)cc3c2)c2cccc3oc4ccccc4c23)cc1]

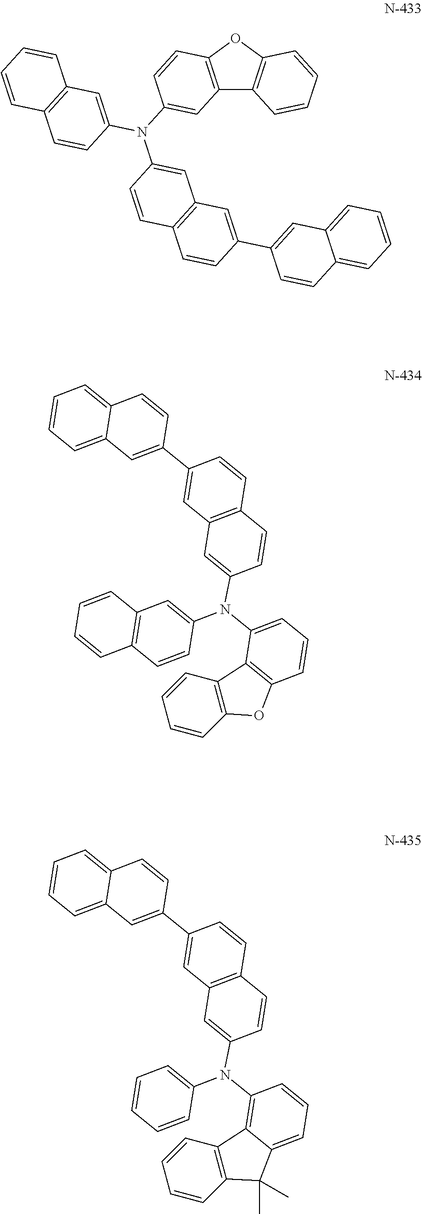 [SMILES: c1ccc2cc(-c3ccc4ccc(N(c5ccc6ccccc6c5)c5ccc6oc7ccccc7c6c5)cc4c3)ccc2c1 ; c1ccc2cc(-c3ccc4ccc(N(c5ccc6ccccc6c5)c5cccc6oc7ccccc7c56)cc4c3)ccc2c1 ; CC1(C)c2ccccc2-c2c(N(c3ccccc3)c3ccc4ccc(-c5ccc6ccccc6c5)cc4c3)cccc21]

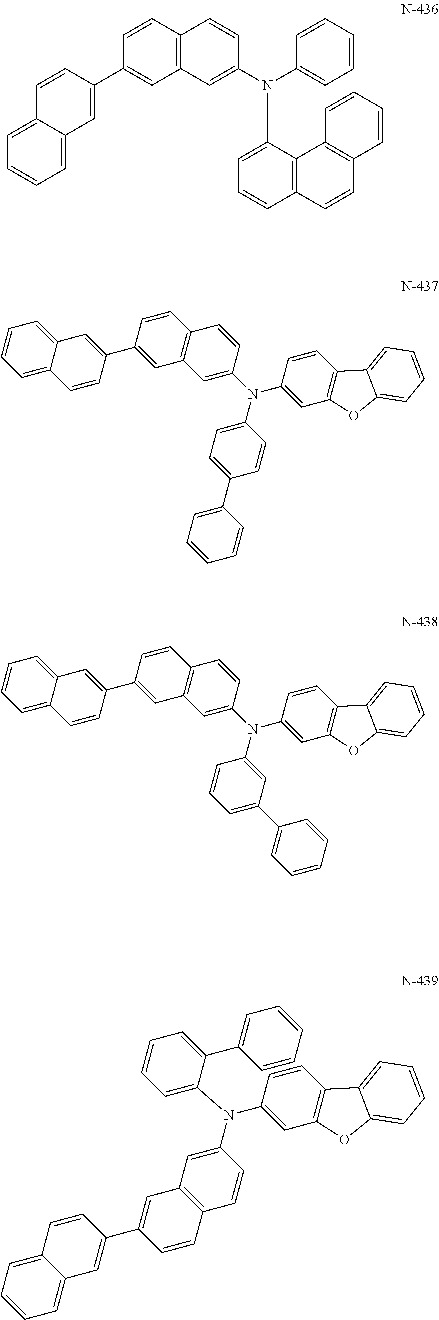 [SMILES: c1ccc(N(c2ccc3ccc(-c4ccc5ccccc5c4)cc3c2)c2cccc3ccc4ccccc4c23)cc1 ; c1ccc(-c2ccc(N(c3ccc4ccc(-c5ccc6ccccc6c5)cc4c3)c3ccc4c(c3)oc3ccccc34)cc2)cc1 ; c1ccc(-c2cccc(N(c3ccc4ccc(-c5ccc6ccccc6c5)cc4c3)c3ccc4c(c3)oc3ccccc34)c2)cc1 ; c1ccc(-c2ccccc2N(c2ccc3ccc(-c4ccc5ccccc5c4)cc3c2)c2ccc3c(c2)oc2ccccc23)cc1]

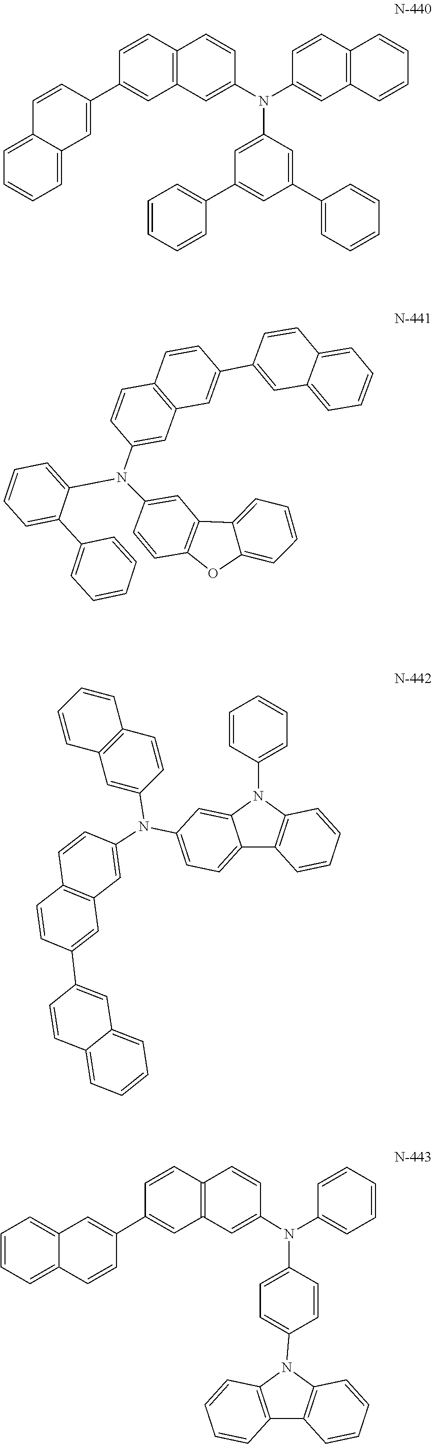 [SMILES: c1ccc(-c2cc(-c3ccccc3)cc(N(c3ccc4ccccc4c3)c3ccc4ccc(-c5ccc6ccccc6c5)cc4c3)c2)cc1 ; c1ccc(-c2ccccc2N(c2ccc3ccc(-c4ccc5ccccc5c4)cc3c2)c2ccc3oc4ccccc4c3c2)cc1 ; c1ccc(-n2c3ccccc3c3ccc(N(c4ccc5ccccc5c4)c4ccc5ccc(-c6ccc7ccccc7c6)cc5c4)cc32)cc1 ; c1ccc(N(c2ccc(-n3c4ccccc4c4ccccc43)cc2)c2ccc3ccc(-c4ccc5ccccc5c4)cc3c2)cc1]

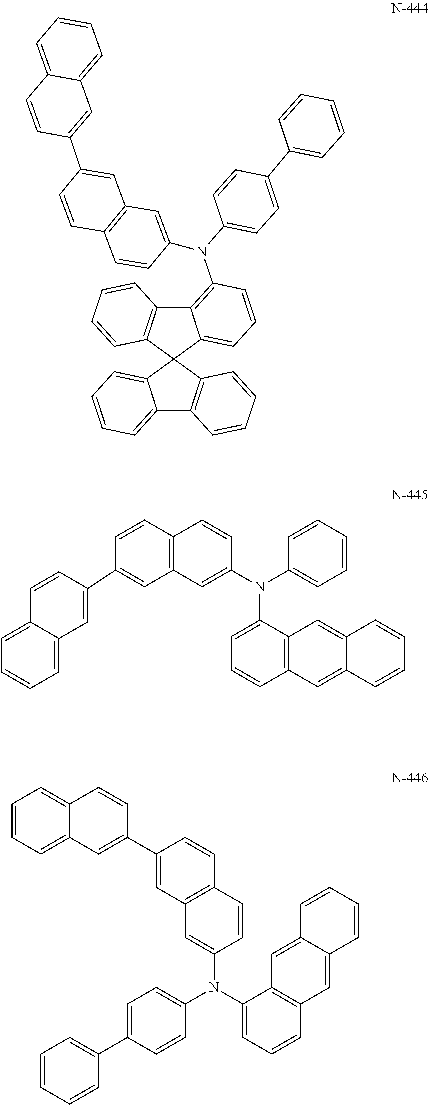 [SMILES: c1ccc(-c2ccc(N(c3ccc4ccc(-c5ccc6ccccc6c5)cc4c3)c3cccc4c3-c3ccccc3C43c4ccccc4-c4ccccc43)cc2)cc1 ; c1ccc(N(c2ccc3ccc(-c4ccc5ccccc5c4)cc3c2)c2cccc3cc4ccccc4cc23)cc1 ; c1ccc(-c2ccc(N(c3ccc4ccc(-c5ccc6ccccc6c5)cc4c3)c3cccc4cc5ccccc5cc34)cc2)cc1]

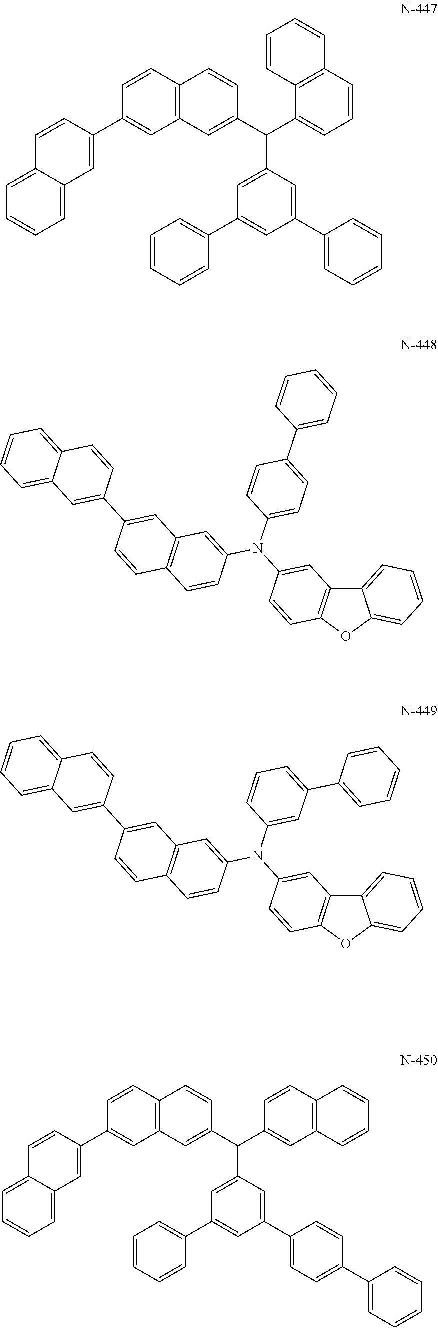

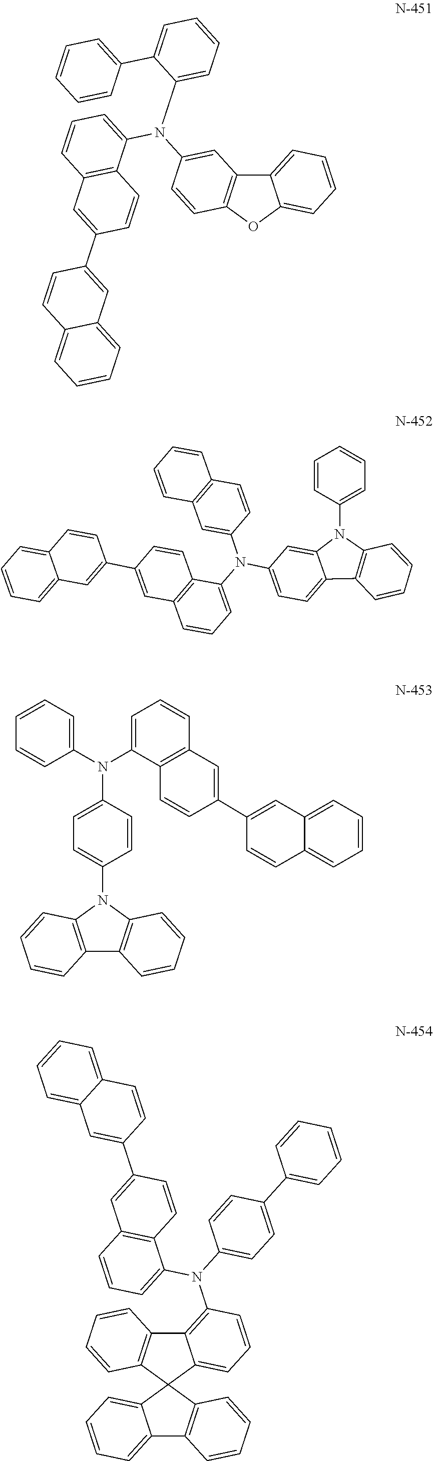 [SMILES: c1ccc(-c2ccccc2N(c2ccc3oc4ccccc4c3c2)c2cccc3cc(-c4ccc5ccccc5c4)ccc23)cc1 ; c1ccc(-n2c3ccccc3c3ccc(N(c4ccc5ccccc5c4)c4cccc5cc(-c6ccc7ccccc7c6)ccc45)cc32)cc1 ; c1ccc(N(c2ccc(-n3c4ccccc4c4ccccc43)cc2)c2cccc3cc(-c4ccc5ccccc5c4)ccc23)cc1 ; c1ccc(-c2ccc(N(c3cccc4c3-c3ccccc3C43c4ccccc4-c4ccccc43)c3cccc4cc(-c5ccc6ccccc6c5)ccc34)cc2)cc1]

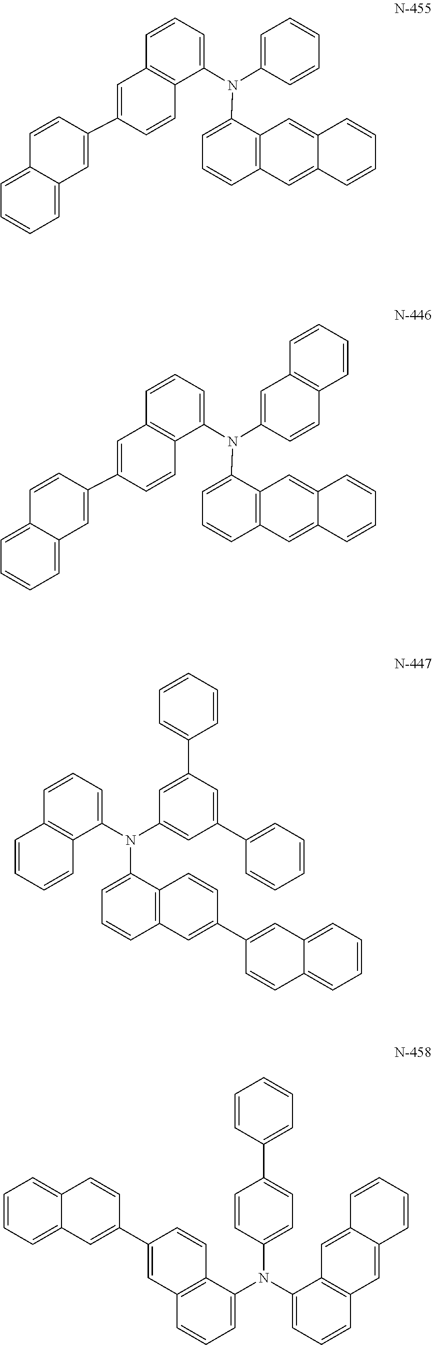 [SMILES: c1ccc(N(c2cccc3cc(-c4ccc5ccccc5c4)ccc23)c2cccc3cc4ccccc4cc23)cc1 ; c1ccc2cc(-c3ccc4c(N(c5ccc6ccccc6c5)c5cccc6cc7ccccc7cc56)cccc4c3)ccc2c1 ; c1ccc(-c2cc(-c3ccccc3)cc(N(c3cccc4ccccc34)c3cccc4cc(-c5ccc6ccccc6c5)ccc34)c2)cc1 ; c1ccc(-c2ccc(N(c3cccc4cc(-c5ccc6ccccc6c5)ccc34)c3cccc4cc5ccccc5cc34)cc2)cc1]

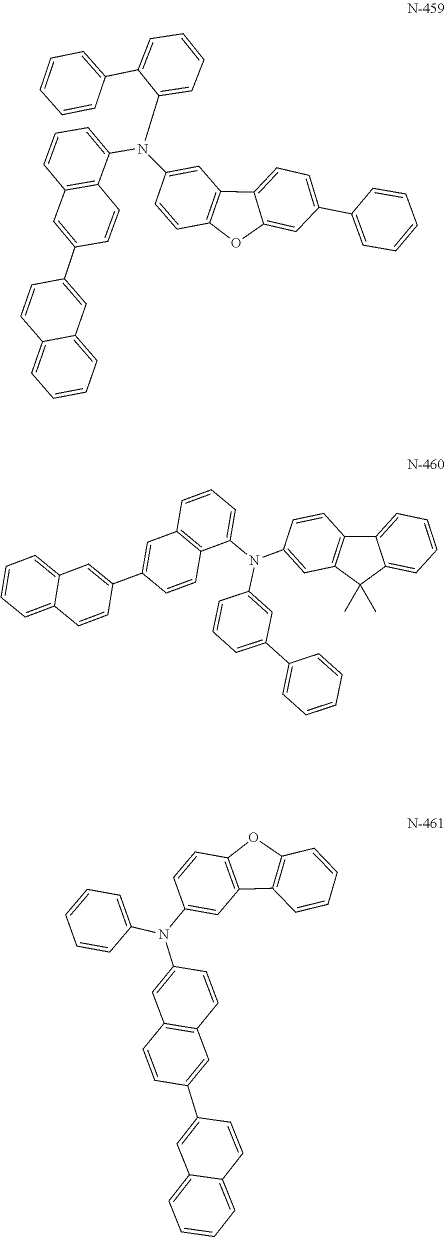 [SMILES: c1ccc(-c2ccc3c(c2)oc2ccc(N(c4ccccc4-c4ccccc4)c4cccc5cc(-c6ccc7ccccc7c6)ccc45)cc23)cc1 ; CC1(C)c2ccccc2-c2ccc(N(c3cccc(-c4ccccc4)c3)c3cccc4cc(-c5ccc6ccccc6c5)ccc34)cc21 ; c1ccc(N(c2ccc3cc(-c4ccc5ccccc5c4)ccc3c2)c2ccc3oc4ccccc4c3c2)cc1]

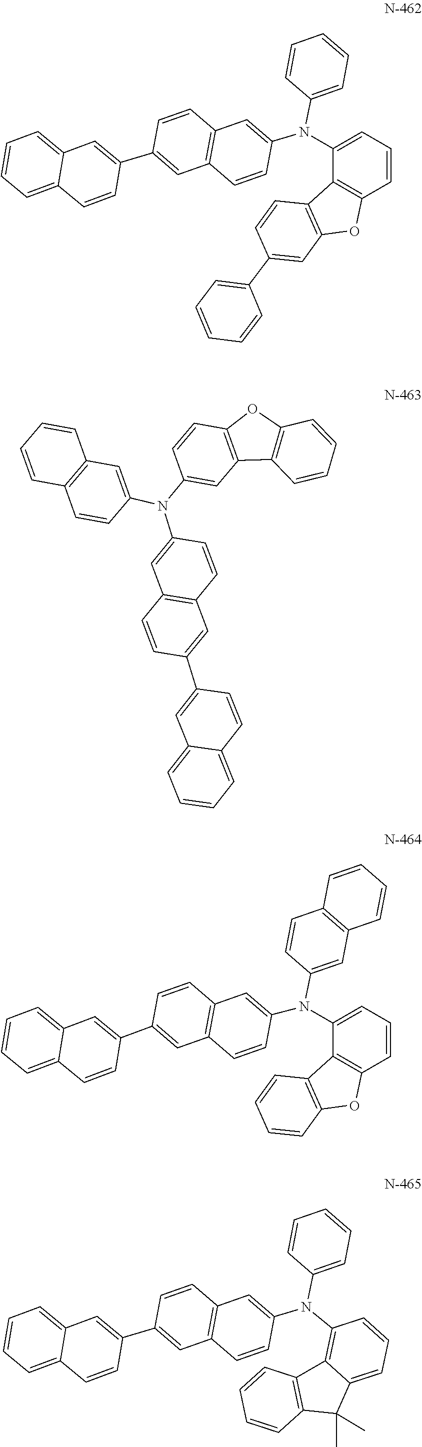 [SMILES: c1ccc(-c2ccc3c(c2)oc2cccc(N(c4ccccc4)c4ccc5cc(-c6ccc7ccccc7c6)ccc5c4)c23)cc1 ; c1ccc2cc(-c3ccc4cc(N(c5ccc6ccccc6c5)c5ccc6oc7ccccc7c6c5)ccc4c3)ccc2c1 ; c1ccc2cc(-c3ccc4cc(N(c5ccc6ccccc6c5)c5cccc6oc7ccccc7c56)ccc4c3)ccc2c1 ; CC1(C)c2ccccc2-c2c(N(c3ccccc3)c3ccc4cc(-c5ccc6ccccc6c5)ccc4c3)cccc21]

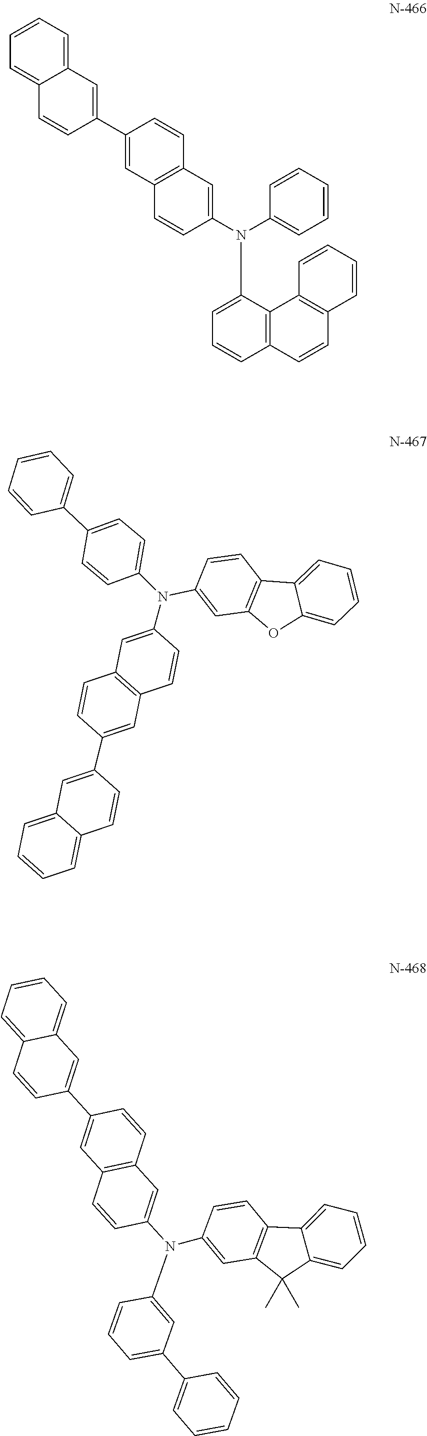 [SMILES: c1ccc(N(c2ccc3cc(-c4ccc5ccccc5c4)ccc3c2)c2cccc3ccc4ccccc4c23)cc1 ; c1ccc(-c2ccc(N(c3ccc4cc(-c5ccc6ccccc6c5)ccc4c3)c3ccc4c(c3)oc3ccccc34)cc2)cc1 ; CC1(C)c2ccccc2-c2ccc(N(c3cccc(-c4ccccc4)c3)c3ccc4cc(-c5ccc6ccccc6c5)ccc4c3)cc21]

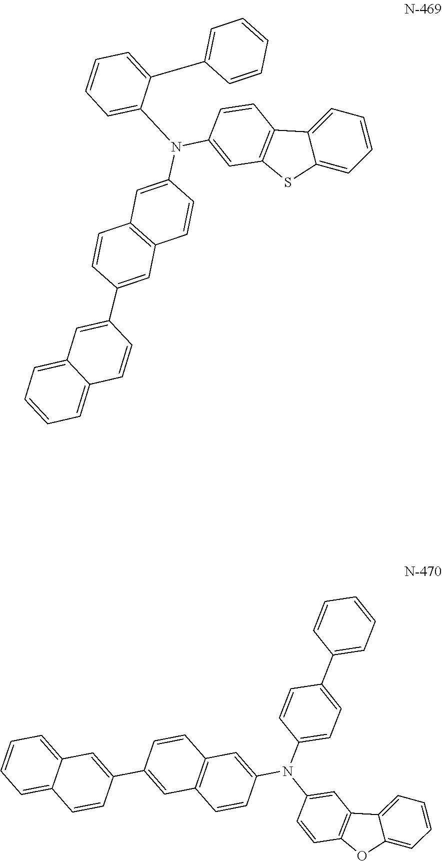 [SMILES: c1ccc(-c2ccccc2N(c2ccc3cc(-c4ccc5ccccc5c4)ccc3c2)c2ccc3c(c2)sc2ccccc23)cc1 ; c1ccc(-c2ccc(N(c3ccc4cc(-c5ccc6ccccc6c5)ccc4c3)c3ccc4oc5ccccc5c4c3)cc2)cc1]

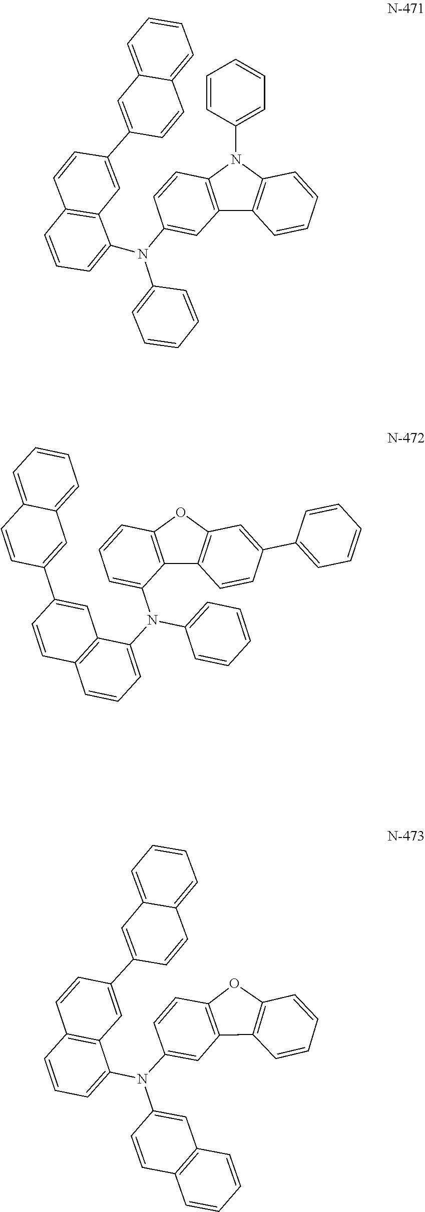 [SMILES: c1ccc(N(c2ccc3c(c2)c2ccccc2n3-c2ccccc2)c2cccc3ccc(-c4ccc5ccccc5c4)cc23)cc1 ; c1ccc(-c2ccc3c(c2)oc2cccc(N(c4ccccc4)c4cccc5ccc(-c6ccc7ccccc7c6)cc45)c23)cc1 ; c1ccc2cc(-c3ccc4cccc(N(c5ccc6ccccc6c5)c5ccc6oc7ccccc7c6c5)c4c3)ccc2c1]

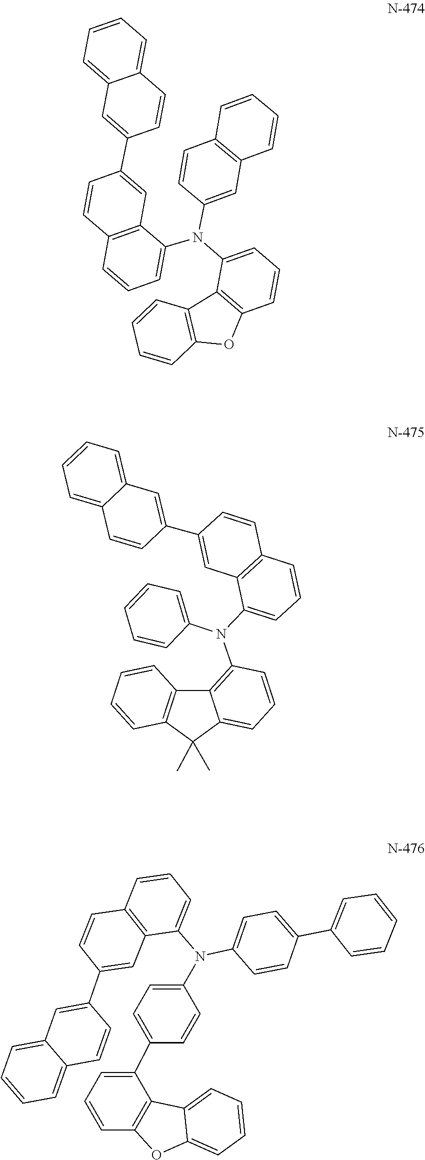 [SMILES: c1ccc2cc(-c3ccc4cccc(N(c5ccc6ccccc6c5)c5cccc6oc7ccccc7c56)c4c3)ccc2c1 ; CC1(C)c2ccccc2-c2c(N(c3ccccc3)c3cccc4ccc(-c5ccc6ccccc6c5)cc34)cccc21 ; c1ccc(-c2ccc(N(c3ccc(-c4cccc5oc6ccccc6c45)cc3)c3cccc4ccc(-c5ccc6ccccc6c5)cc34)cc2)cc1]

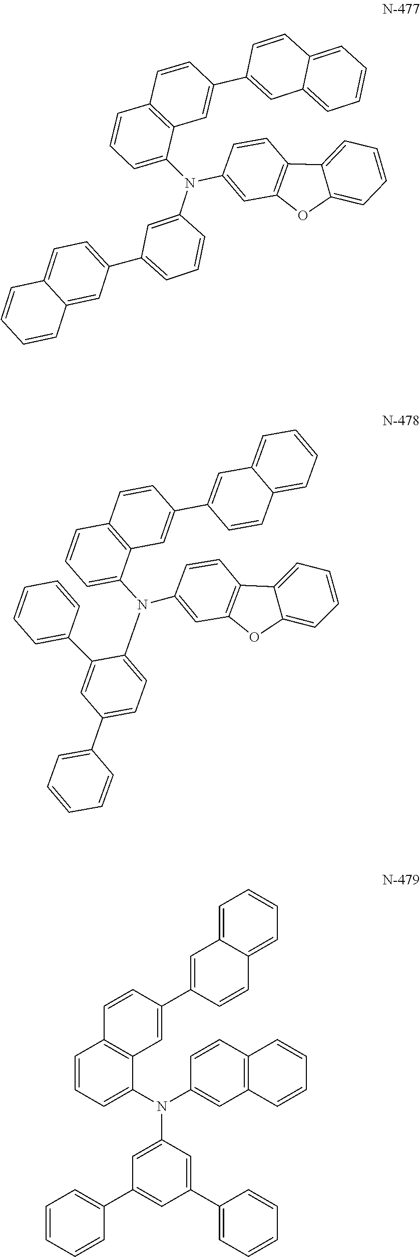 [SMILES: c1cc(-c2ccc3ccccc3c2)cc(N(c2ccc3c(c2)oc2ccccc23)c2cccc3ccc(-c4ccc5ccccc5c4)cc23)c1 ; c1ccc(-c2ccc(N(c3ccc4c(c3)oc3ccccc34)c3cccc4ccc(-c5ccc6ccccc6c5)cc34)c(-c3ccccc3)c2)cc1 ; c1ccc(-c2cc(-c3ccccc3)cc(N(c3ccc4ccccc4c3)c3cccc4ccc(-c5ccc6ccccc6c5)cc34)c2)cc1]

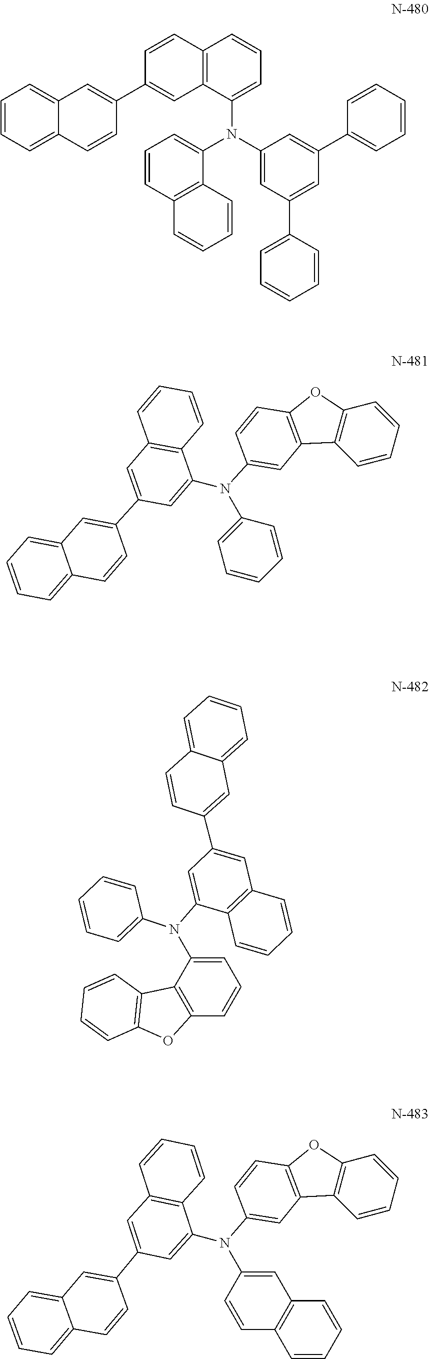 [SMILES: c1ccc(-c2cc(-c3ccccc3)cc(N(c3cccc4ccccc34)c3cccc4ccc(-c5ccc6ccccc6c5)cc34)c2)cc1 ; c1ccc(N(c2ccc3oc4ccccc4c3c2)c2cc(-c3ccc4ccccc4c3)cc3ccccc23)cc1 ; c1ccc(N(c2cc(-c3ccc4ccccc4c3)cc3ccccc23)c2cccc3oc4ccccc4c23)cc1 ; c1ccc2cc(-c3cc(N(c4ccc5ccccc5c4)c4ccc5oc6ccccc6c5c4)c4ccccc4c3)ccc2c1]

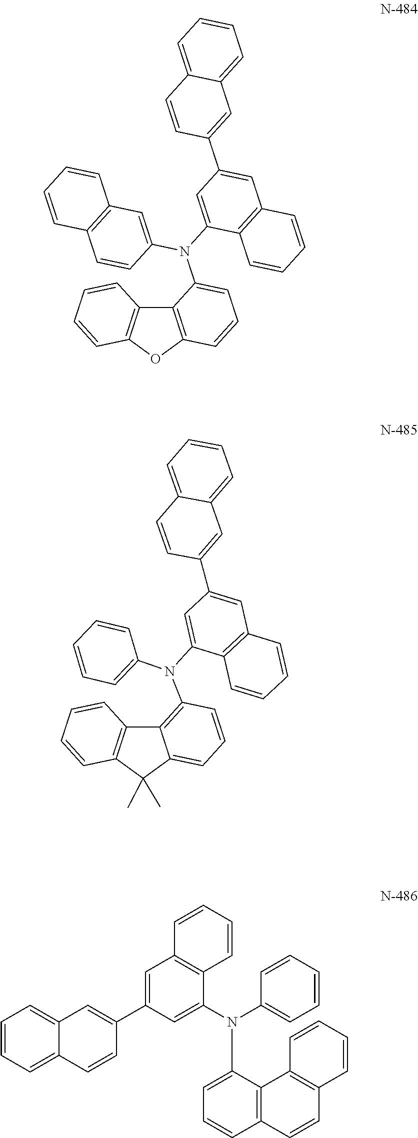 [SMILES: c1ccc2cc(-c3cc(N(c4ccc5ccccc5c4)c4cccc5oc6ccccc6c45)c4ccccc4c3)ccc2c1 ; CC1(C)c2ccccc2-c2c(N(c3ccccc3)c3cc(-c4ccc5ccccc5c4)cc4ccccc34)cccc21 ; c1ccc(N(c2cc(-c3ccc4ccccc4c3)cc3ccccc23)c2cccc3ccc4ccccc4c23)cc1]

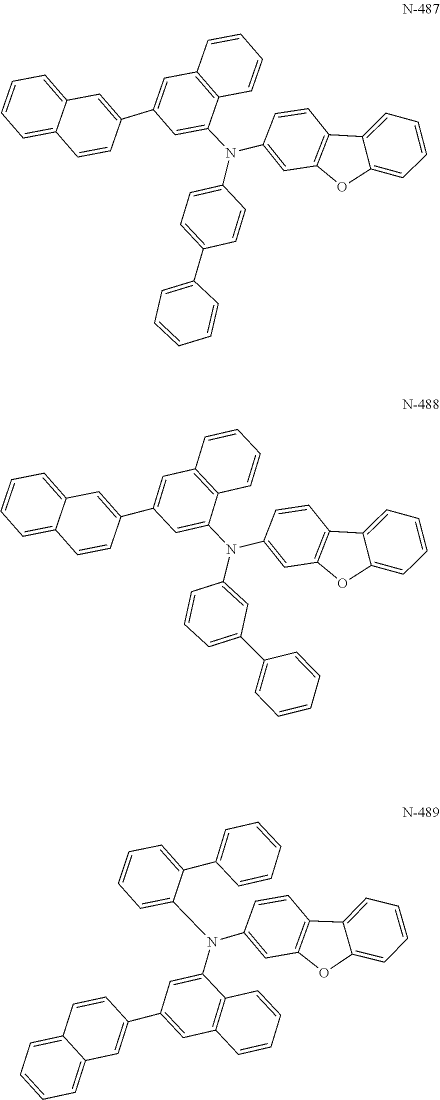 [SMILES: c1ccc(-c2ccc(N(c3ccc4c(c3)oc3ccccc34)c3cc(-c4ccc5ccccc5c4)cc4ccccc34)cc2)cc1 ; c1ccc(-c2cccc(N(c3ccc4c(c3)oc3ccccc34)c3cc(-c4ccc5ccccc5c4)cc4ccccc34)c2)cc1 ; c1ccc(-c2ccccc2N(c2ccc3c(c2)oc2ccccc23)c2cc(-c3ccc4ccccc4c3)cc3ccccc23)cc1]

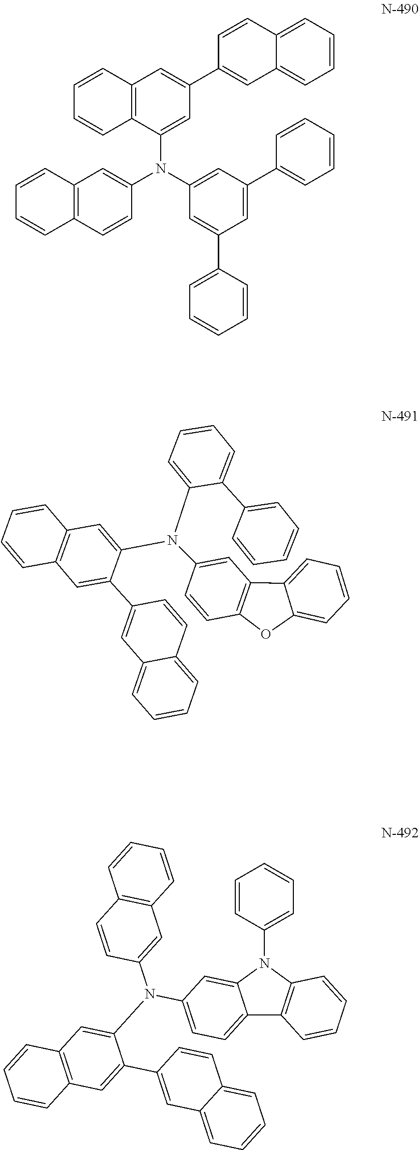 [SMILES: c1ccc(-c2cc(-c3ccccc3)cc(N(c3ccc4ccccc4c3)c3cc(-c4ccc5ccccc5c4)cc4ccccc34)c2)cc1 ; c1ccc(-c2ccccc2N(c2ccc3oc4ccccc4c3c2)c2cc3ccccc3cc2-c2ccc3ccccc3c2)cc1 ; c1ccc(-n2c3ccccc3c3ccc(N(c4ccc5ccccc5c4)c4cc5ccccc5cc4-c4ccc5ccccc5c4)cc32)cc1]

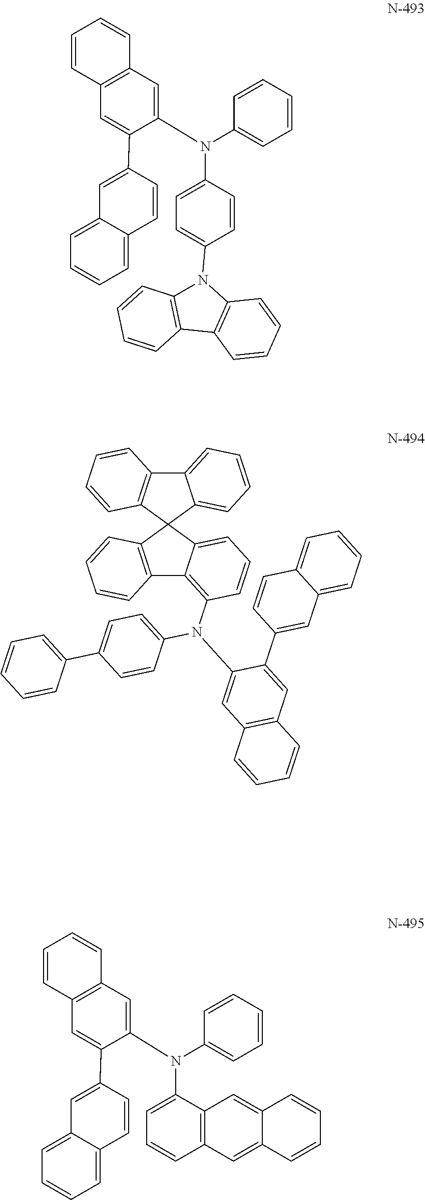 [SMILES: c1ccc(N(c2ccc(-n3c4ccccc4c4ccccc43)cc2)c2cc3ccccc3cc2-c2ccc3ccccc3c2)cc1 ; c1ccc(-c2ccc(N(c3cc4ccccc4cc3-c3ccc4ccccc4c3)c3cccc4c3-c3ccccc3C43c4ccccc4-c4ccccc43)cc2)cc1 ; c1ccc(N(c2cc3ccccc3cc2-c2ccc3ccccc3c2)c2cccc3cc4ccccc4cc23)cc1]

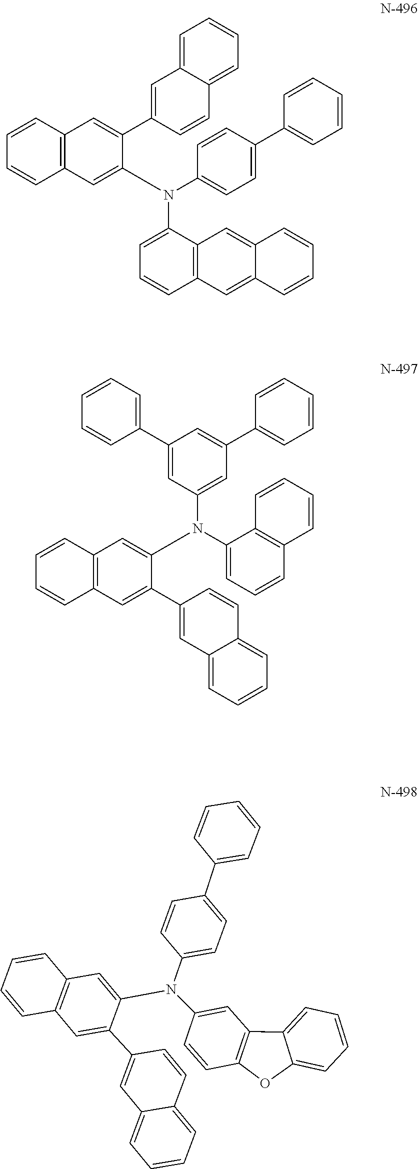 [SMILES: c1ccc(-c2ccc(N(c3cc4ccccc4cc3-c3ccc4ccccc4c3)c3cccc4cc5ccccc5cc34)cc2)cc1 ; c1ccc(-c2cc(-c3ccccc3)cc(N(c3cc4ccccc4cc3-c3ccc4ccccc4c3)c3cccc4ccccc34)c2)cc1 ; c1ccc(-c2ccc(N(c3ccc4oc5ccccc5c4c3)c3cc4ccccc4cc3-c3ccc4ccccc4c3)cc2)cc1]

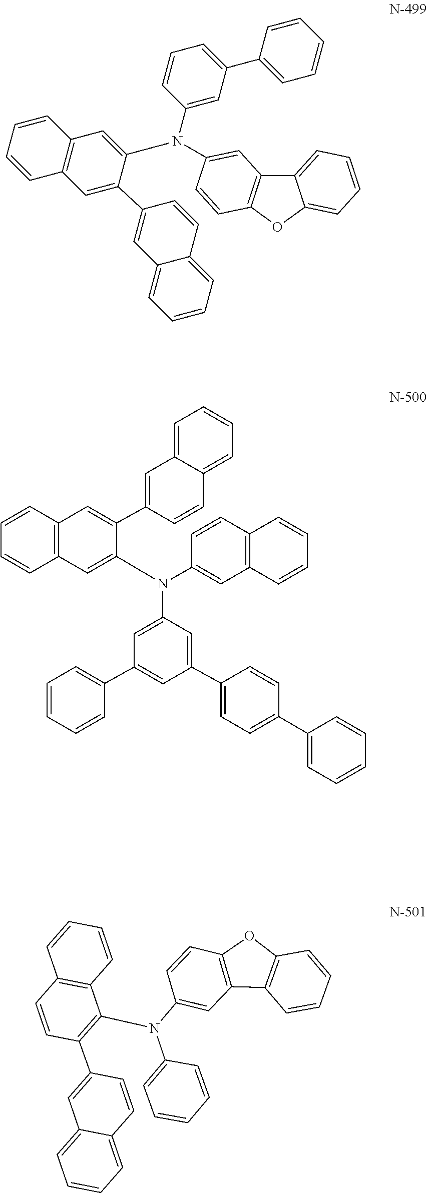 [SMILES: c1ccc(-c2cccc(N(c3ccc4oc5ccccc5c4c3)c3cc4ccccc4cc3-c3ccc4ccccc4c3)c2)cc1 ; c1ccc(-c2ccc(-c3cc(-c4ccccc4)cc(N(c4ccc5ccccc5c4)c4cc5ccccc5cc4-c4ccc5ccccc5c4)c3)cc2)cc1 ; c1ccc(N(c2ccc3oc4ccccc4c3c2)c2c(-c3ccc4ccccc4c3)ccc3ccccc23)cc1]

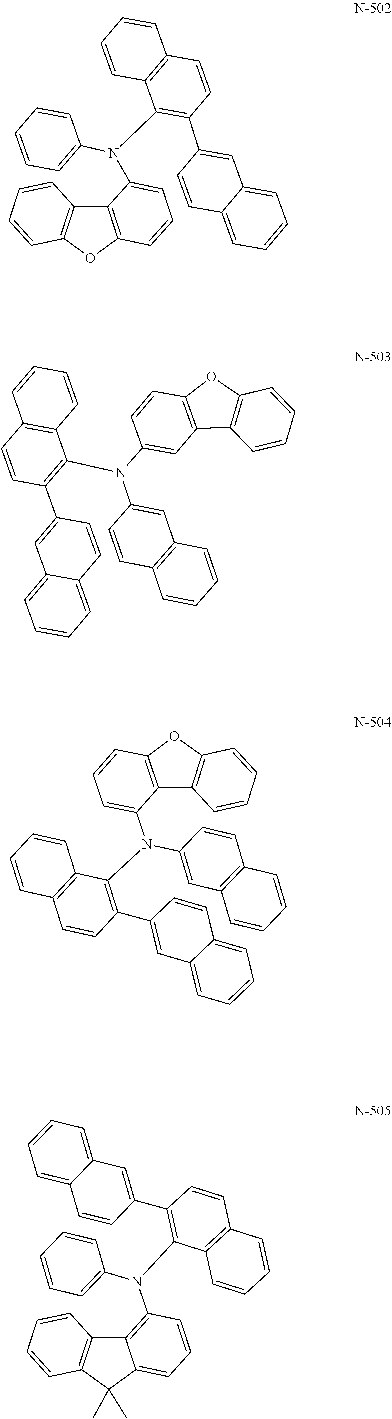 [SMILES: c1ccc(N(c2c(-c3ccc4ccccc4c3)ccc3ccccc23)c2cccc3oc4ccccc4c23)cc1 ; c1ccc2cc(-c3ccc4ccccc4c3N(c3ccc4ccccc4c3)c3ccc4oc5ccccc5c4c3)ccc2c1 ; c1ccc2cc(-c3ccc4ccccc4c3N(c3ccc4ccccc4c3)c3cccc4oc5ccccc5c34)ccc2c1 ; CC1(C)c2ccccc2-c2c(N(c3ccccc3)c3c(-c4ccc5ccccc5c4)ccc4ccccc34)cccc21]

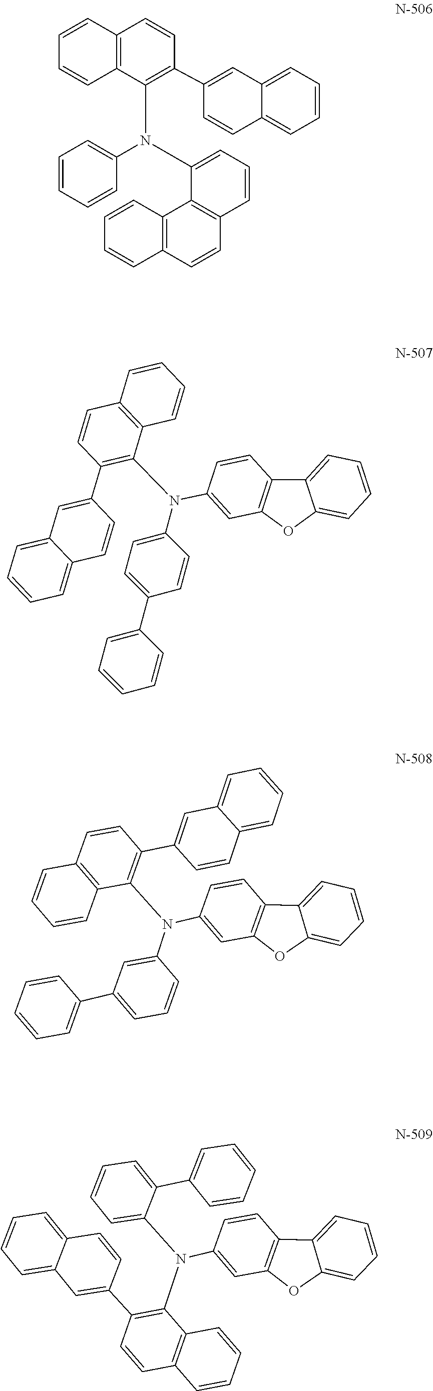 [SMILES: c1ccc(N(c2c(-c3ccc4ccccc4c3)ccc3ccccc23)c2cccc3ccc4ccccc4c23)cc1 ; c1ccc(-c2ccc(N(c3ccc4c(c3)oc3ccccc34)c3c(-c4ccc5ccccc5c4)ccc4ccccc34)cc2)cc1 ; c1ccc(-c2cccc(N(c3ccc4c(c3)oc3ccccc34)c3c(-c4ccc5ccccc5c4)ccc4ccccc34)c2)cc1 ; c1ccc(-c2ccccc2N(c2ccc3c(c2)oc2ccccc23)c2c(-c3ccc4ccccc4c3)ccc3ccccc23)cc1]

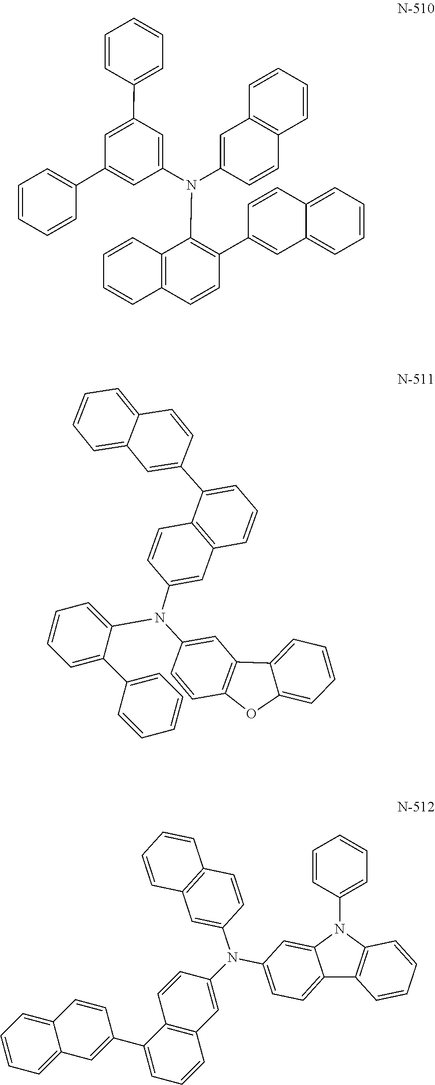 [SMILES: c1ccc(-c2cc(-c3ccccc3)cc(N(c3ccc4ccccc4c3)c3c(-c4ccc5ccccc5c4)ccc4ccccc34)c2)cc1 ; c1ccc(-c2ccccc2N(c2ccc3c(-c4ccc5ccccc5c4)cccc3c2)c2ccc3oc4ccccc4c3c2)cc1 ; c1ccc(-n2c3ccccc3c3ccc(N(c4ccc5ccccc5c4)c4ccc5c(-c6ccc7ccccc7c6)cccc5c4)cc32)cc1]

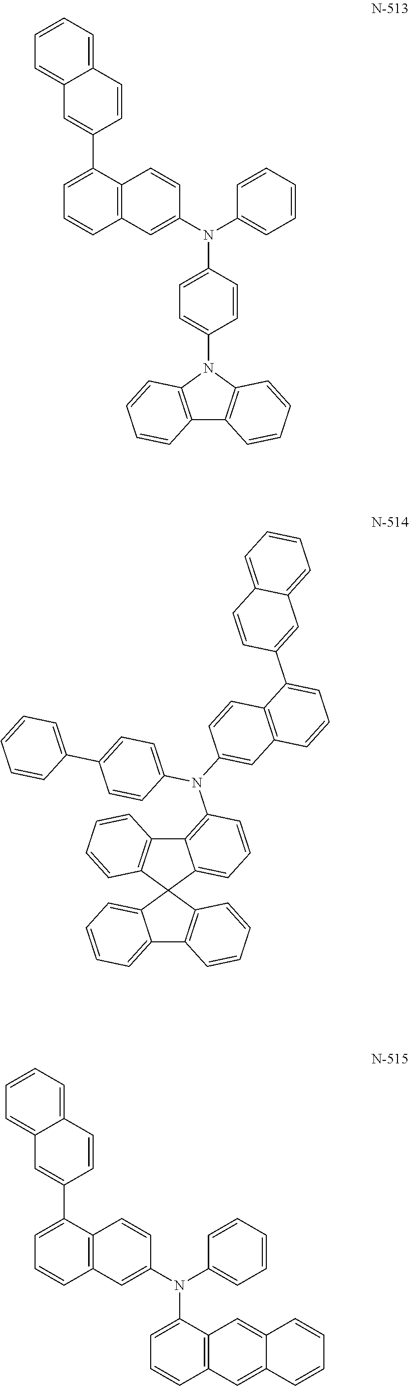 [SMILES: c1ccc(N(c2ccc(-n3c4ccccc4c4ccccc43)cc2)c2ccc3c(-c4ccc5ccccc5c4)cccc3c2)cc1 ; c1ccc(-c2ccc(N(c3ccc4c(-c5ccc6ccccc6c5)cccc4c3)c3cccc4c3-c3ccccc3C43c4ccccc4-c4ccccc43)cc2)cc1 ; c1ccc(N(c2ccc3c(-c4ccc5ccccc5c4)cccc3c2)c2cccc3cc4ccccc4cc23)cc1]

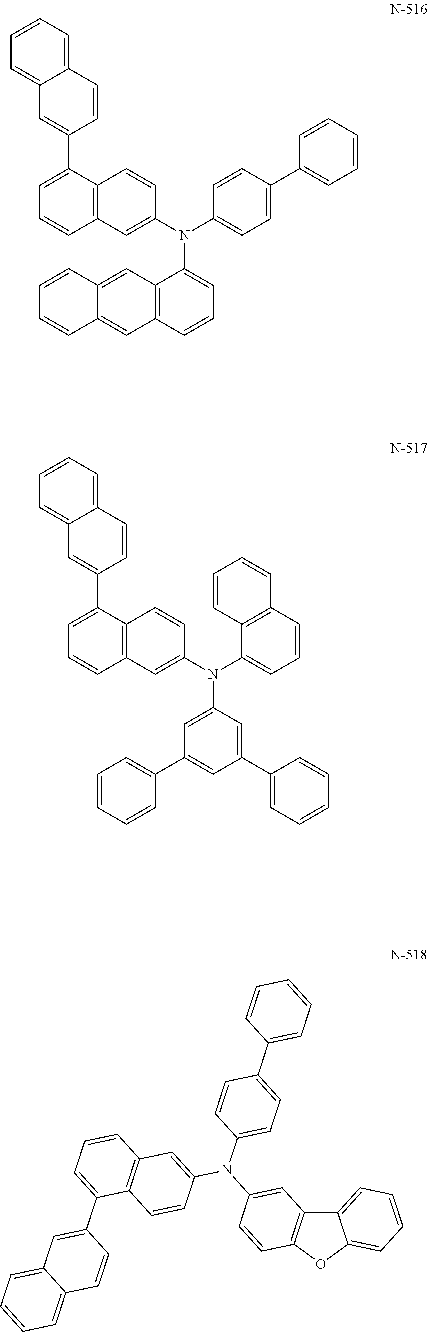 [SMILES: c1ccc(-c2ccc(N(c3ccc4c(-c5ccc6ccccc6c5)cccc4c3)c3cccc4cc5ccccc5cc34)cc2)cc1 ; c1ccc(-c2cc(-c3ccccc3)cc(N(c3ccc4c(-c5ccc6ccccc6c5)cccc4c3)c3cccc4ccccc34)c2)cc1 ; c1ccc(-c2ccc(N(c3ccc4c(-c5ccc6ccccc6c5)cccc4c3)c3ccc4oc5ccccc5c4c3)cc2)cc1]

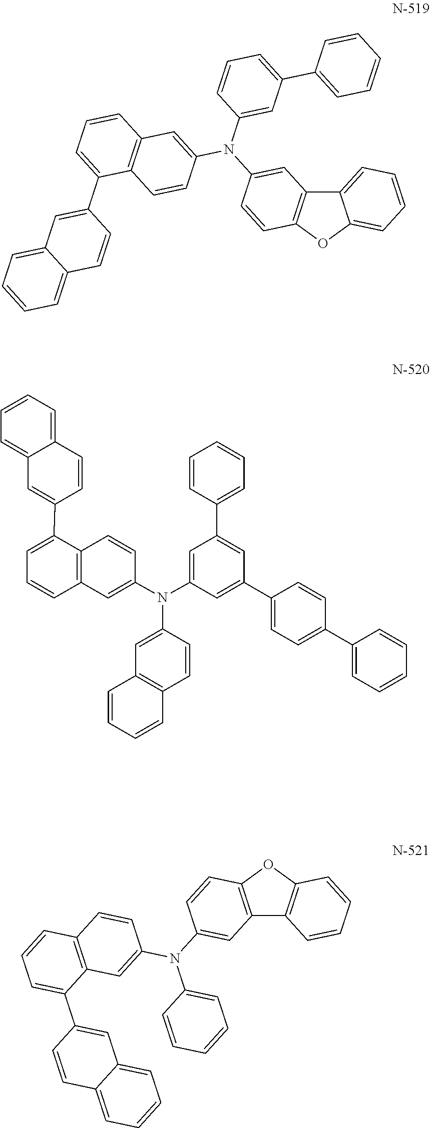 [SMILES: c1ccc(-c2cccc(N(c3ccc4c(-c5ccc6ccccc6c5)cccc4c3)c3ccc4oc5ccccc5c4c3)c2)cc1 ; c1ccc(-c2ccc(-c3cc(-c4ccccc4)cc(N(c4ccc5ccccc5c4)c4ccc5c(-c6ccc7ccccc7c6)cccc5c4)c3)cc2)cc1 ; c1ccc(N(c2ccc3cccc(-c4ccc5ccccc5c4)c3c2)c2ccc3oc4ccccc4c3c2)cc1]

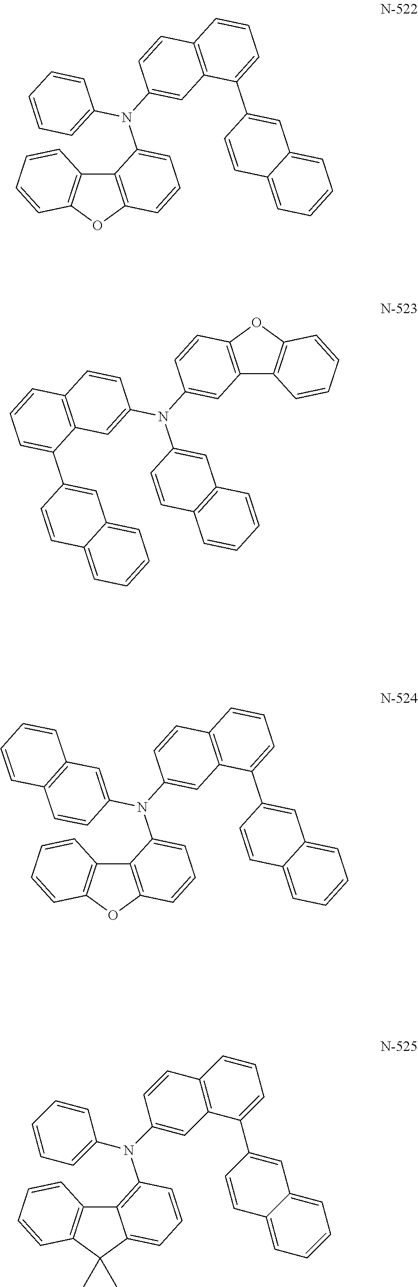 [SMILES: c1ccc(N(c2ccc3cccc(-c4ccc5ccccc5c4)c3c2)c2cccc3oc4ccccc4c23)cc1 ; c1ccc2cc(-c3cccc4ccc(N(c5ccc6ccccc6c5)c5ccc6oc7ccccc7c6c5)cc34)ccc2c1 ; c1ccc2cc(-c3cccc4ccc(N(c5ccc6ccccc6c5)c5cccc6oc7ccccc7c56)cc34)ccc2c1 ; CC1(C)c2ccccc2-c2c(N(c3ccccc3)c3ccc4cccc(-c5ccc6ccccc6c5)c4c3)cccc21]

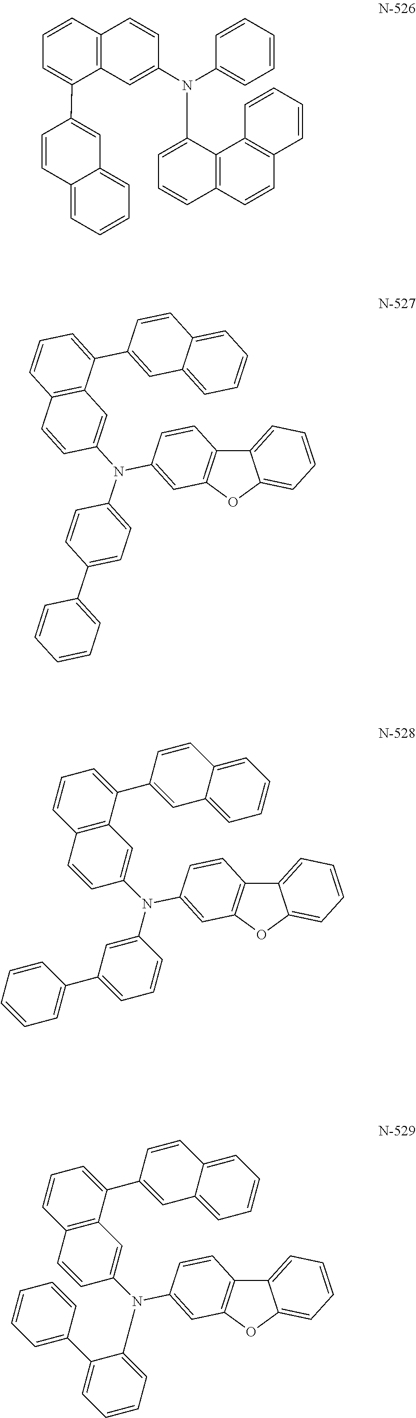 [SMILES: c1ccc(N(c2ccc3cccc(-c4ccc5ccccc5c4)c3c2)c2cccc3ccc4ccccc4c23)cc1 ; c1ccc(-c2ccc(N(c3ccc4c(c3)oc3ccccc34)c3ccc4cccc(-c5ccc6ccccc6c5)c4c3)cc2)cc1 ; c1ccc(-c2cccc(N(c3ccc4c(c3)oc3ccccc34)c3ccc4cccc(-c5ccc6ccccc6c5)c4c3)c2)cc1 ; c1ccc(-c2ccccc2N(c2ccc3c(c2)oc2ccccc23)c2ccc3cccc(-c4ccc5ccccc5c4)c3c2)cc1]

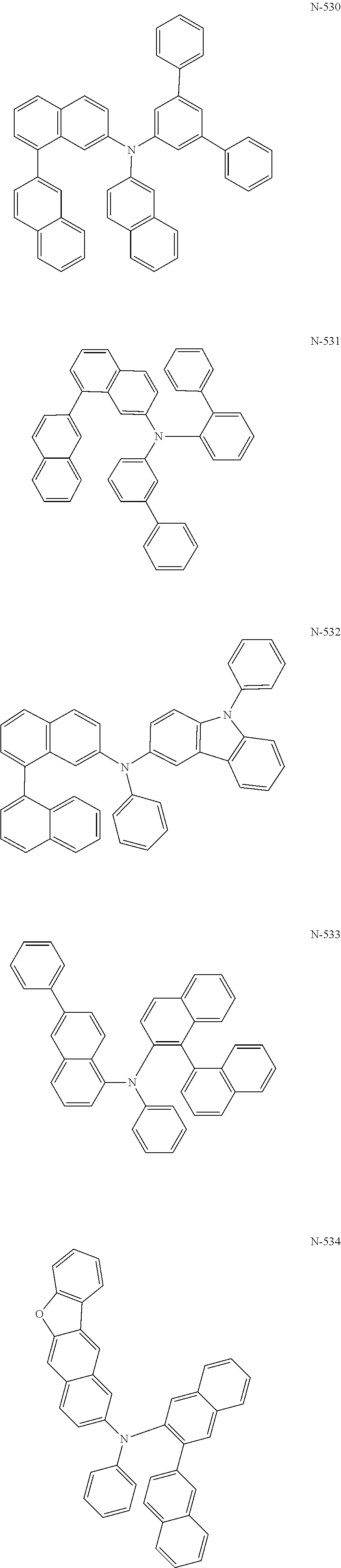 [SMILES: c1ccc(-c2cc(-c3ccccc3)cc(N(c3ccc4ccccc4c3)c3ccc4cccc(-c5ccc6ccccc6c5)c4c3)c2)cc1 ; c1ccc(-c2cccc(N(c3ccc4cccc(-c5ccc6ccccc6c5)c4c3)c3ccccc3-c3ccccc3)c2)cc1 ; c1ccc(N(c2ccc3cccc(-c4cccc5ccccc45)c3c2)c2ccc3c(c2)c2ccccc2n3-c2ccccc2)cc1 ; c1ccc(-c2ccc3c(N(c4ccccc4)c4ccc5ccccc5c4-c4cccc5ccccc45)cccc3c2)cc1 ; c1ccc(N(c2ccc3cc4oc5ccccc5c4cc3c2)c2cc3ccccc3cc2-c2ccc3ccccc3c2)cc1]

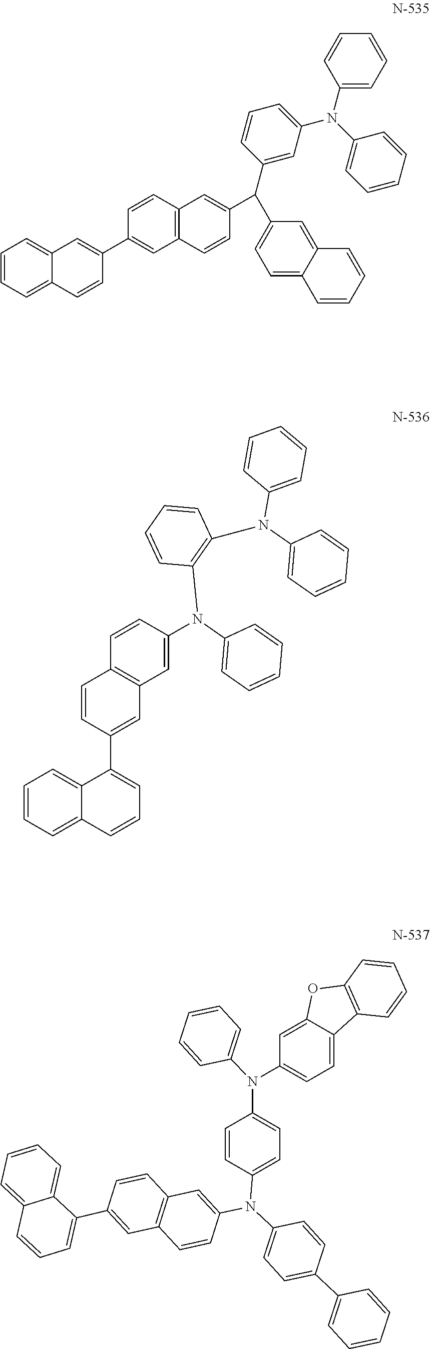 [SMILES: c1ccc(N(c2ccccc2)c2cccc(C(c3ccc4ccccc4c3)c3ccc4cc(-c5ccc6ccccc6c5)ccc4c3)c2)cc1 ; c1ccc(N(c2ccccc2)c2ccccc2N(c2ccccc2)c2ccc3ccc(-c4cccc5ccccc45)cc3c2)cc1 ; c1ccc(-c2ccc(N(c3ccc(N(c4ccccc4)c4ccc5c(c4)oc4ccccc45)cc3)c3ccc4cc(-c5cccc6ccccc56)ccc4c3)cc2)cc1]

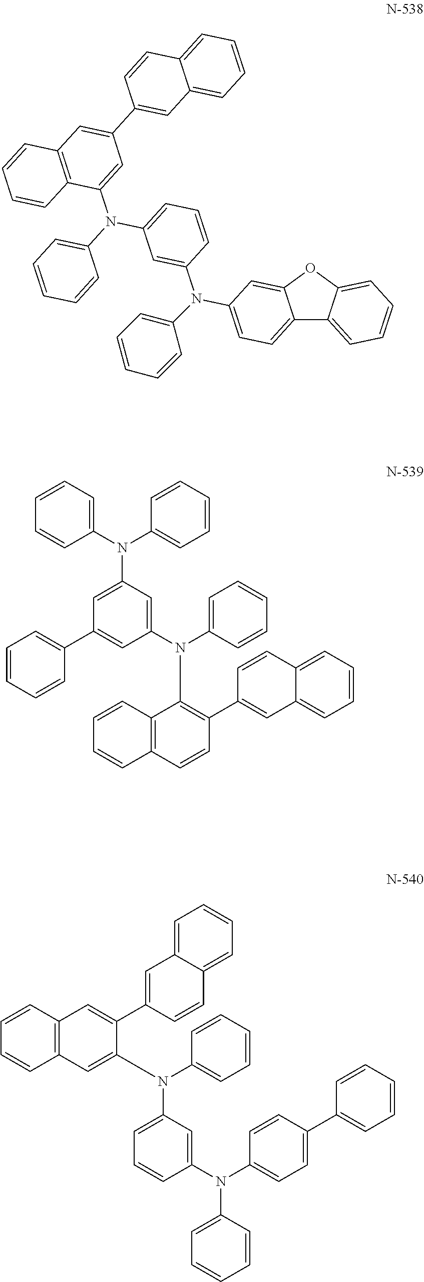 [SMILES: c1ccc(N(c2cccc(N(c3ccccc3)c3cc(-c4ccc5ccccc5c4)cc4ccccc34)c2)c2ccc3c(c2)oc2ccccc23)cc1 ; c1ccc(-c2cc(N(c3ccccc3)c3ccccc3)cc(N(c3ccccc3)c3c(-c4ccc5ccccc5c4)ccc4ccccc34)c2)cc1 ; c1ccc(-c2ccc(N(c3ccccc3)c3cccc(N(c4ccccc4)c4cc5ccccc5cc4-c4ccc5ccccc5c4)c3)cc2)cc1]

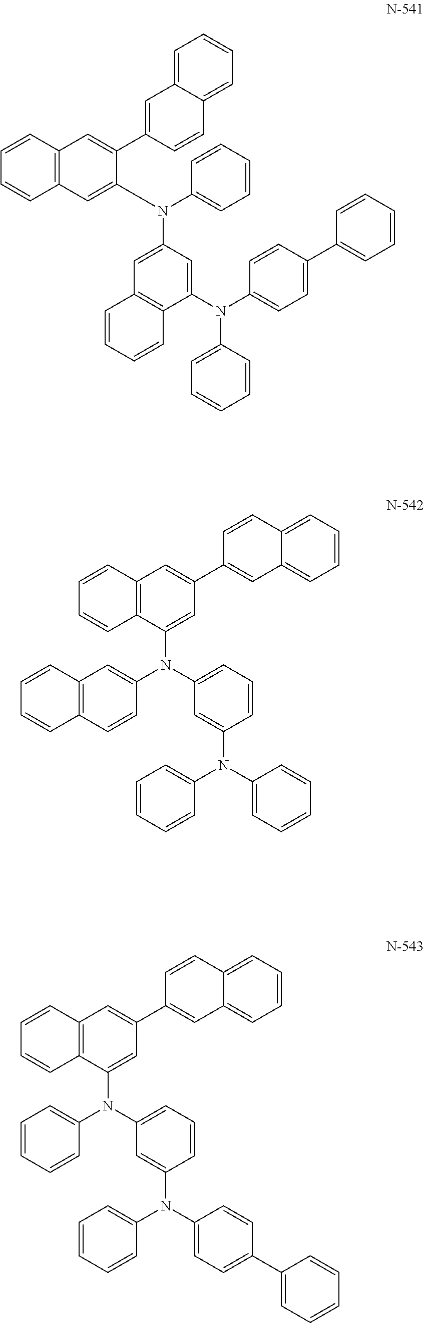 [SMILES: c1ccc(-c2ccc(N(c3ccccc3)c3cc(N(c4ccccc4)c4cc5ccccc5cc4-c4ccc5ccccc5c4)cc4ccccc34)cc2)cc1 ; c1ccc(N(c2ccccc2)c2cccc(N(c3ccc4ccccc4c3)c3cc(-c4ccc5ccccc5c4)cc4ccccc34)c2)cc1 ; c1ccc(-c2ccc(N(c3ccccc3)c3cccc(N(c4ccccc4)c4cc(-c5ccc6ccccc6c5)cc5ccccc45)c3)cc2)cc1]

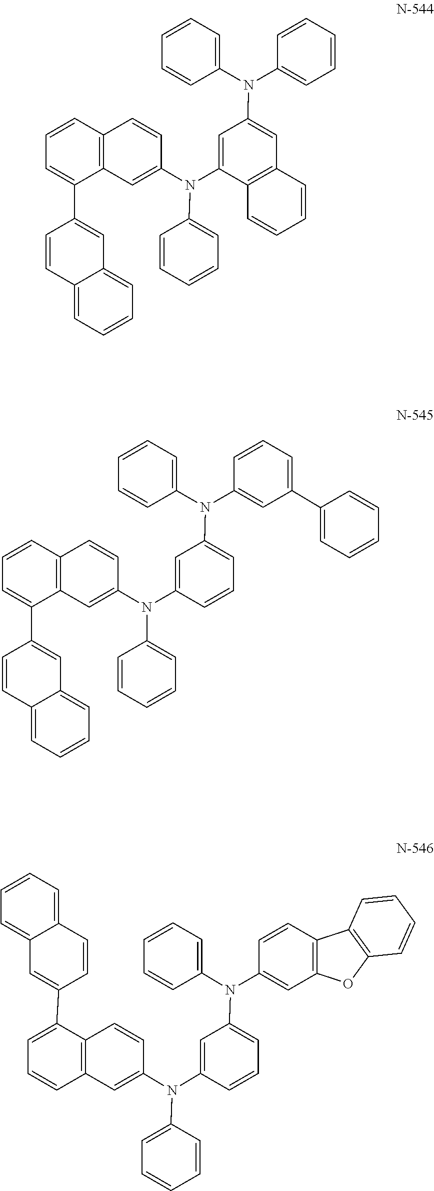 [SMILES: c1ccc(N(c2ccccc2)c2cc(N(c3ccccc3)c3ccc4cccc(-c5ccc6ccccc6c5)c4c3)c3ccccc3c2)cc1 ; c1ccc(-c2cccc(N(c3ccccc3)c3cccc(N(c4ccccc4)c4ccc5cccc(-c6ccc7ccccc7c6)c5c4)c3)c2)cc1 ; c1ccc(N(c2cccc(N(c3ccccc3)c3ccc4c(c3)oc3ccccc34)c2)c2ccc3c(-c4ccc5ccccc5c4)cccc3c2)cc1]

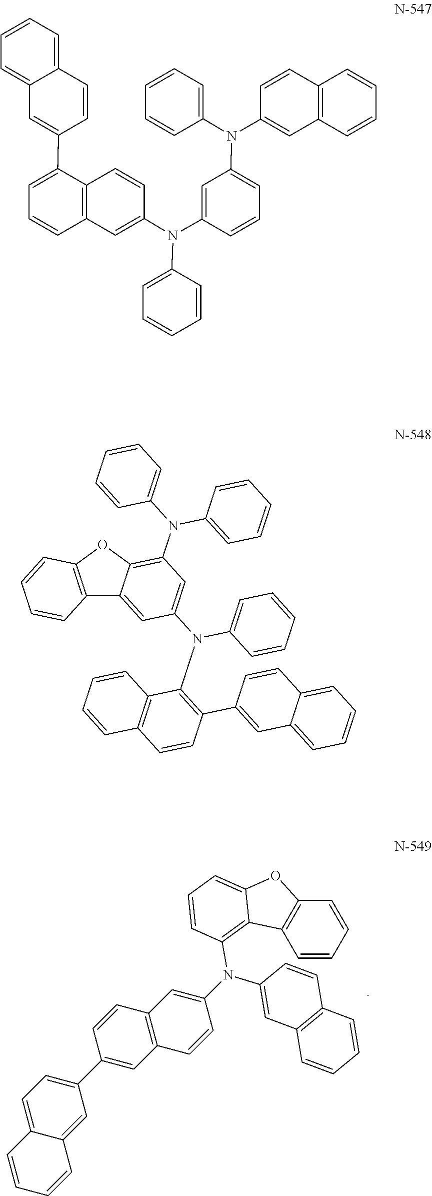 [SMILES: c1ccc(N(c2cccc(N(c3ccccc3)c3ccc4c(-c5ccc6ccccc6c5)cccc4c3)c2)c2ccc3ccccc3c2)cc1 ; c1ccc(N(c2cc(N(c3ccccc3)c3ccccc3)c3oc4ccccc4c3c2)c2c(-c3ccc4ccccc4c3)ccc3ccccc23)cc1 ; c1ccc2cc(-c3ccc4cc(N(c5ccc6ccccc6c5)c5cccc6oc7ccccc7c56)ccc4c3)ccc2c1]

The present application also provides an organic electroluminescent material composition, comprising the above organic electroluminescent compound N and compound M, the compound M are compounds represented by Formula (2):

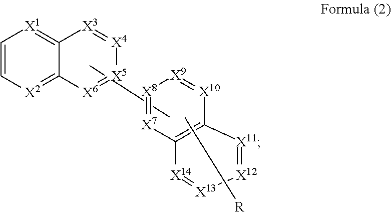

R is selected from the following structure:

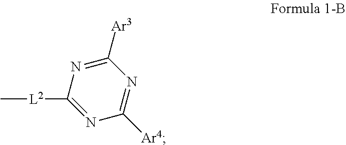

wherein, X¹-X¹⁴ are each independently selected from N or CR⁸, and R⁸ is selected from hydrogen or deuterium;
L² is each independently selected from a connecting bond, substituted or unsubstituted C6-C30 arylenyl, and substituted or unsubstituted C3-C30 heteroarylenyl;
Ar³-Ar⁴ are each independently selected from substituted or unsubstituted C6-C30 aryl, substituted or unsubstituted C3-C30 heteroaryl, substituted or unsubstituted C6-C60 arylamine group, and substituted or unsubstituted C3-C60 heteroarylamine group;
the substituents in the substituted C6-C30 arylenyl, the substituted C3-C30 heteroarylenyl, the substituted C6-C30 aryl, the substituted C3-C30 heteroaryl, the substituted C6-C60 arylamine group, and the substituted C3-C60 heteroarylamine group are selected from one of or a combination of two of deuterium, halogen, cyano, C1-C6 alkyl, C3-C30 cycloalkyl, C6-C30 aryl, and C3-C30 heteroaryl.

Preferably, in Formula (2), X¹-X¹⁴ are all selected from CR⁸, and R⁸ is selected from hydrogen or deuterium;
preferably, X¹-X¹⁴ are all selected from CR⁸, and R⁸ is hydrogen;
preferably, any one of X¹-X⁶ is selected from N, and the remaining is CR⁸;
preferably, any one of X¹-X⁶ is selected from N, and the remaining is CR⁸; any one of X⁷-X¹⁴ is selected from N, and the remaining is CR⁸;
wherein, R⁸ is each present independently, and can be the same or different; R⁸ is defined the same as above; and
preferably, Ar¹-Ar⁴ are each independently selected from substituted or unsubstituted A groups;
the A group comprises: phenyl, naphthyl, biphenyl, triphenyl, phenanthryl, fluoranthenyl, triphenylenyl, fluorenyl, dimethylfluorenyl, diphenylfluorenyl, spirobifluorenyl, benzodimethylfluorenyl, benzodiphenylfluorenyl, benzospirobifluorenyl, benzofuryl, dibenzofuryl, naphthobenzofuryl, dinaphthalofuryl, benzothienyl, dibenzothienyl, naphthobenzothienyl, carbazolyl, phenylcarbazolyl, benzophenylcarbazolyl, dibenzophenylcarbazolyl, biphenylcarbazolyl, phenanthrobenzofuryl, dibenzofuranofuryl, and phenylcarbazolobenzofuryl;
wherein, the substituent of the substituted A group is selected from one of or a combination of two of deuterium, halogen, cyano, C1-C6 alkyl, C3-C30 cycloalkyl, C6-C30 aryl, C3-C30 heteroaryl, C6-C60 arylamine group, and C3-C60 heteroarylamine group;
preferably, Ar³-Ar⁴ are each independently selected from phenyl, naphthyl, biphenyl, triphenyl, phenanthryl, fluoranthenyl, triphenylenyl, fluorenyl, dimethylfluorenyl, diphenylfluorenyl, spirobifluorenyl, benzodimethylfluorenyl, benzodiphenylfluorenyl, benzospirobifluorenyl, dibenzofuryl, naphthobenzofuryl, dibenzothienyl, naphthobenzothienyl, carbazolyl, phenylcarbazolyl, benzocarbazolyl, dibenzocarbazolyl;
preferably, L² is each independently selected from a connecting bond, and substituted or unsubstituted C6-C18 arylenyl;
preferably, L² is selected from a connecting bond and phenylenyl.

Preferably, the compound M has a structure as shown in any one of Formula 2-1 to Formula 2-28:

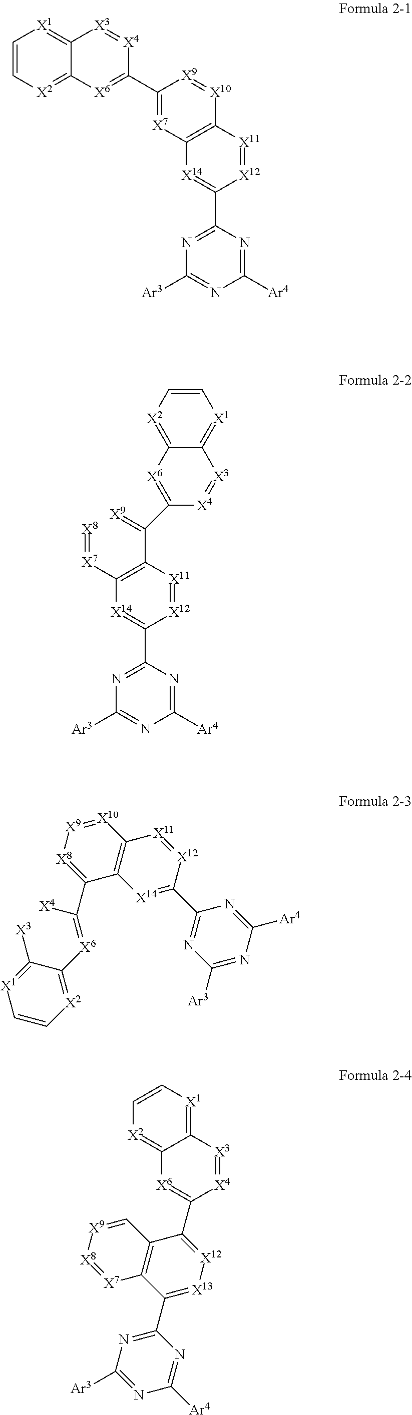

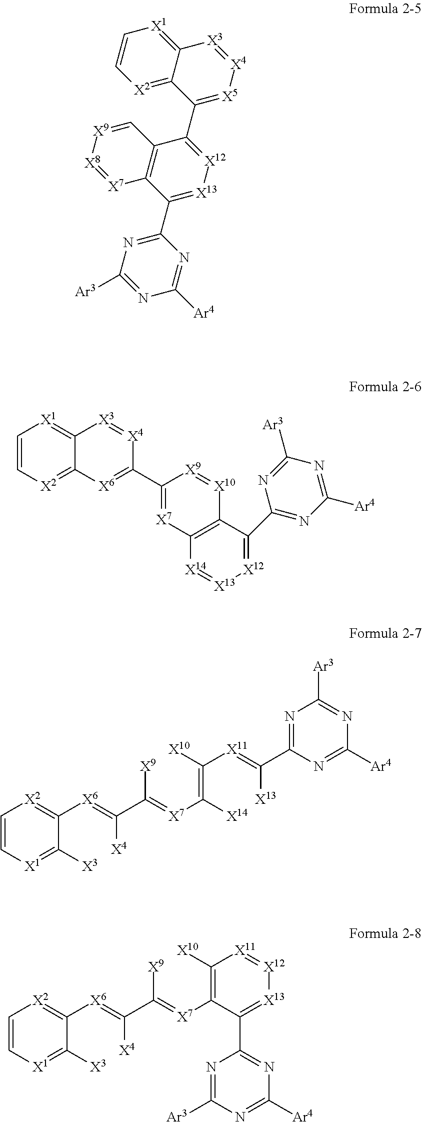

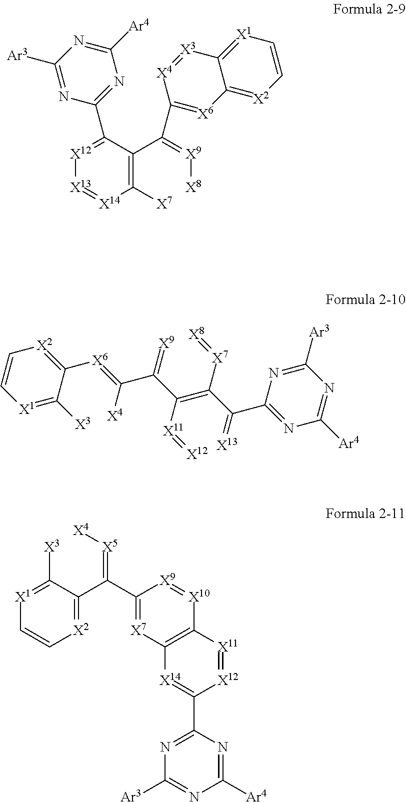

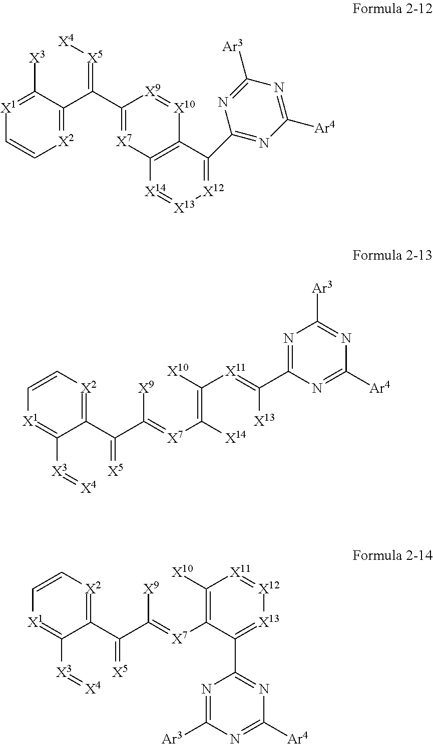

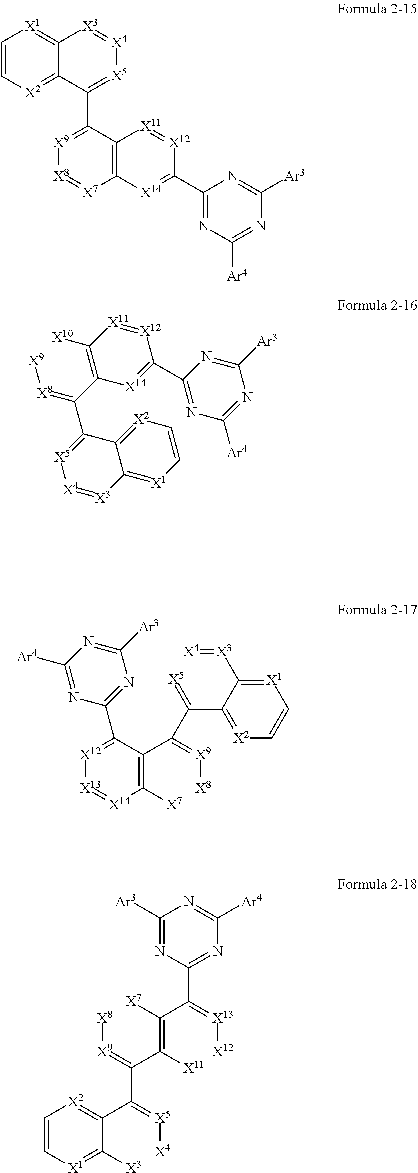

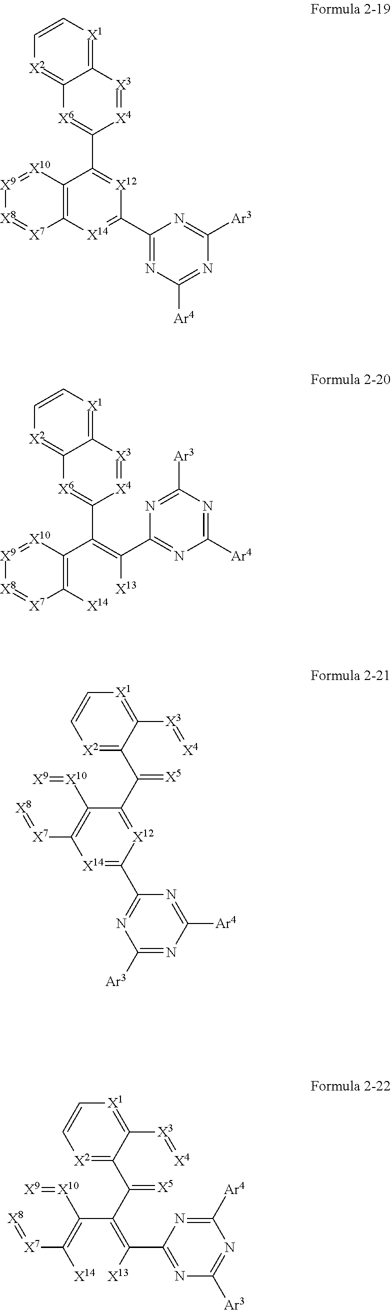

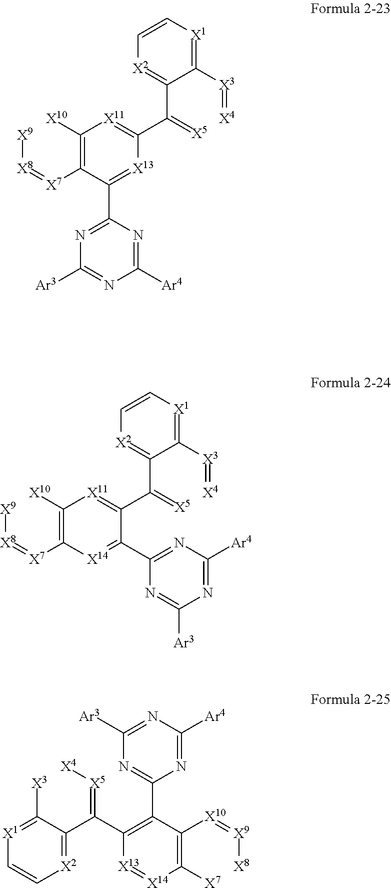

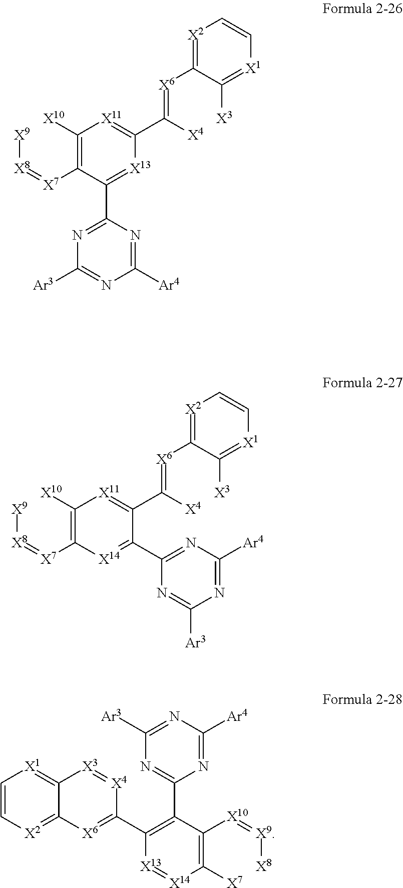

wherein Ar³-Ar⁴ is defined the same as above.

Preferably, the structure of compound M is shown as any one in M-1 to M-619:

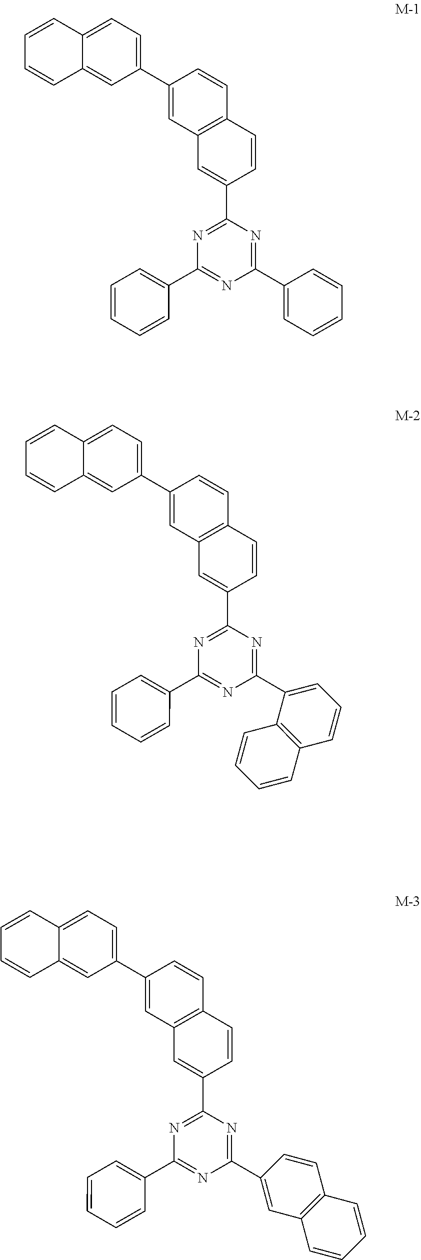 [SMILES: c1ccc(-c2nc(-c3ccccc3)nc(-c3ccc4ccc(-c5ccc6ccccc6c5)cc4c3)n2)cc1 ; c1ccc(-c2nc(-c3ccc4ccc(-c5ccc6ccccc6c5)cc4c3)nc(-c3cccc4ccccc34)n2)cc1 ; c1ccc(-c2nc(-c3ccc4ccccc4c3)nc(-c3ccc4ccc(-c5ccc6ccccc6c5)cc4c3)n2)cc1]

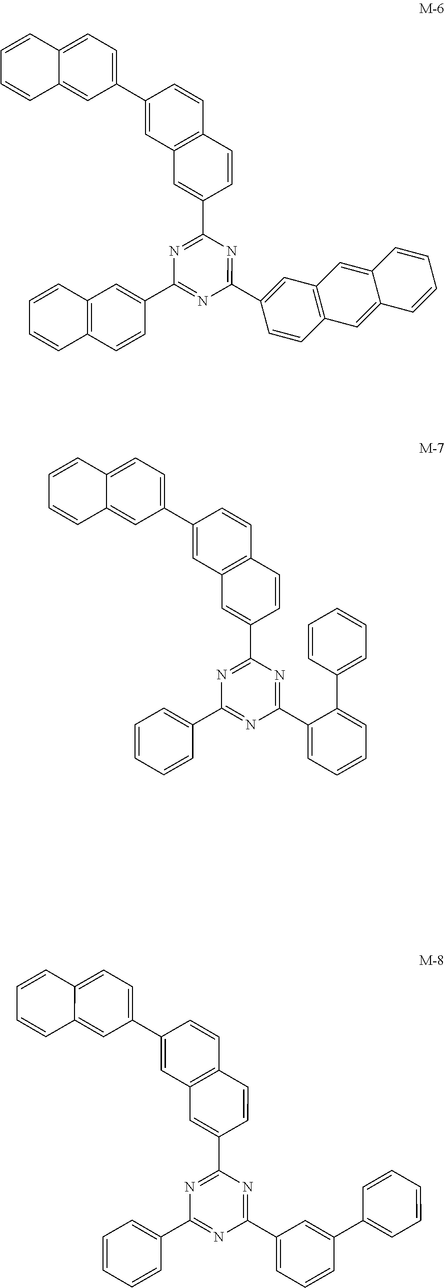 [SMILES: c1ccc2cc(-c3ccc4ccc(-c5nc(-c6ccc7ccccc7c6)nc(-c6ccc7cc8ccccc8cc7c6)n5)cc4c3)ccc2c1 ; c1ccc(-c2nc(-c3ccc4ccc(-c5ccc6ccccc6c5)cc4c3)nc(-c3ccccc3-c3ccccc3)n2)cc1 ; c1ccc(-c2cccc(-c3nc(-c4ccccc4)nc(-c4ccc5ccc(-c6ccc7ccccc7c6)cc5c4)n3)c2)cc1]

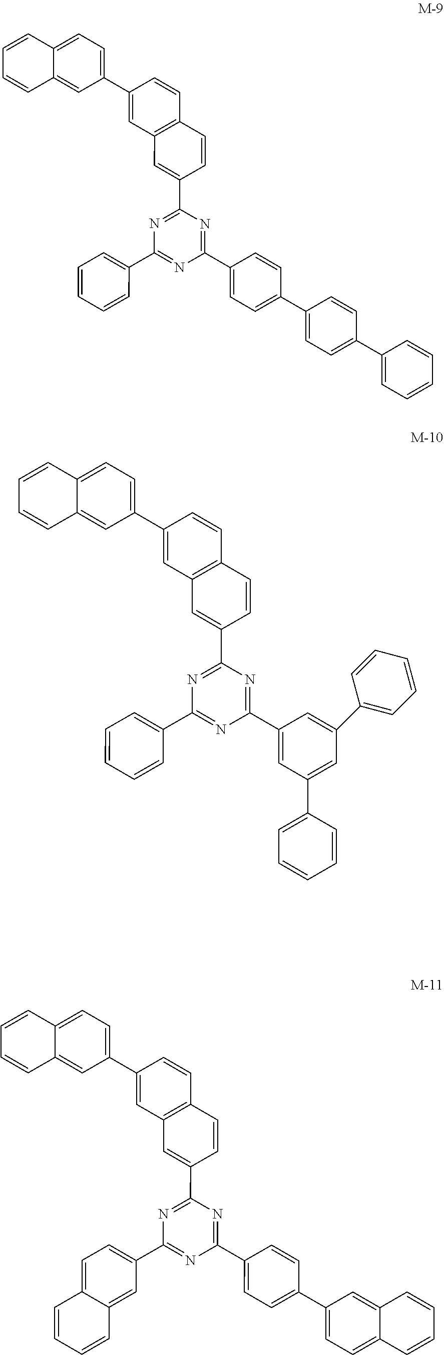 [SMILES: c1ccc(-c2ccc(-c3ccc(-c4nc(-c5ccccc5)nc(-c5ccc6ccc(-c7ccc8ccccc8c7)cc6c5)n4)cc3)cc2)cc1 ; c1ccc(-c2cc(-c3ccccc3)cc(-c3nc(-c4ccccc4)nc(-c4ccc5ccc(-c6ccc7ccccc7c6)cc5c4)n3)c2)cc1 ; c1ccc2cc(-c3ccc(-c4nc(-c5ccc6ccccc6c5)nc(-c5ccc6ccc(-c7ccc8ccccc8c7)cc6c5)n4)cc3)ccc2c1]

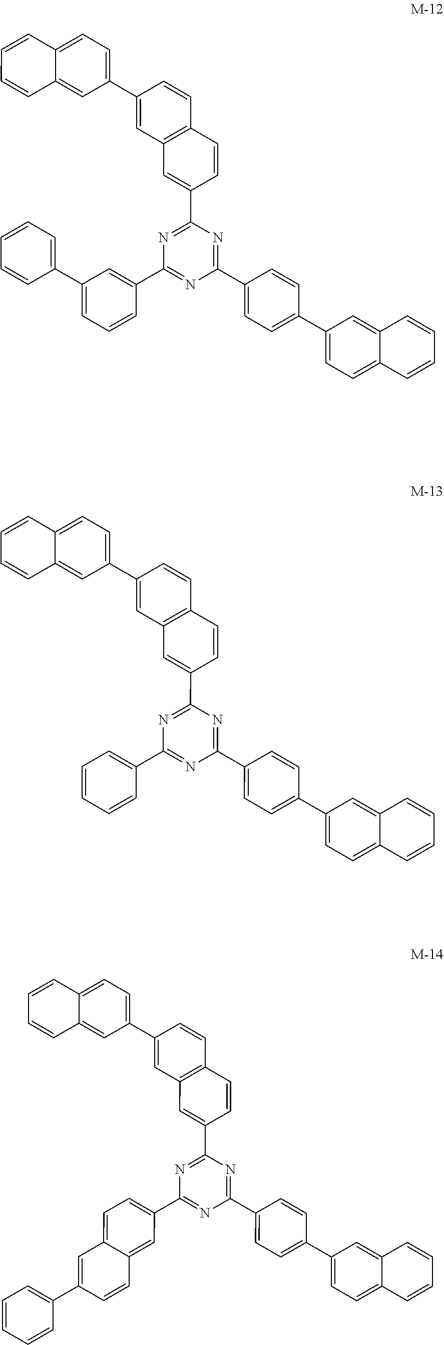 [SMILES: c1ccc(-c2cccc(-c3nc(-c4ccc(-c5ccc6ccccc6c5)cc4)nc(-c4ccc5ccc(-c6ccc7ccccc7c6)cc5c4)n3)c2)cc1 ; c1ccc(-c2nc(-c3ccc(-c4ccc5ccccc5c4)cc3)nc(-c3ccc4ccc(-c5ccc6ccccc6c5)cc4c3)n2)cc1 ; c1ccc(-c2ccc3cc(-c4nc(-c5ccc(-c6ccc7ccccc7c6)cc5)nc(-c5ccc6ccc(-c7ccc8ccccc8c7)cc6c5)n4)ccc3c2)cc1]

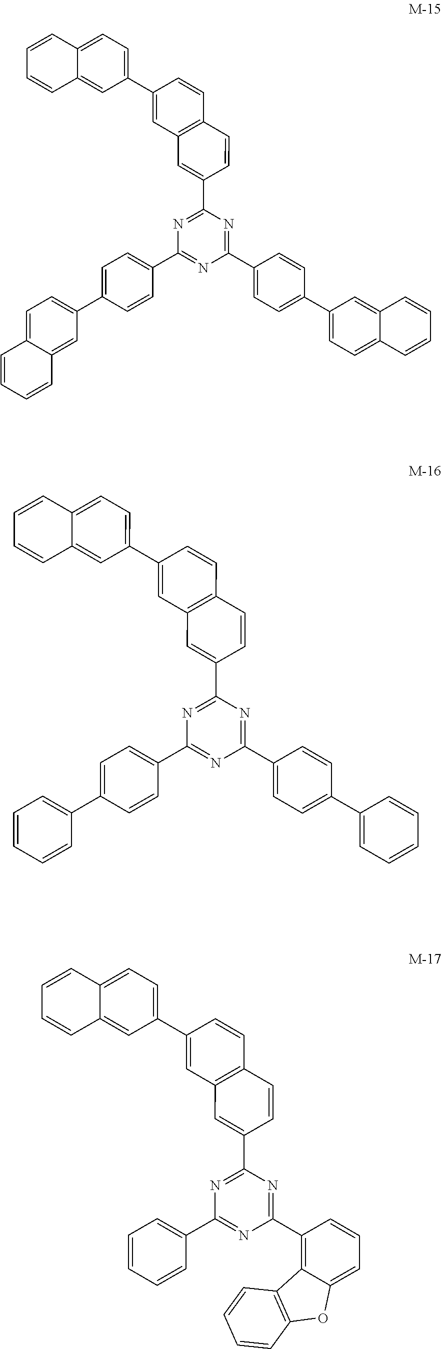 [SMILES: c1ccc2cc(-c3ccc(-c4nc(-c5ccc(-c6ccc7ccccc7c6)cc5)nc(-c5ccc6ccc(-c7ccc8ccccc8c7)cc6c5)n4)cc3)ccc2c1 ; c1ccc(-c2ccc(-c3nc(-c4ccc(-c5ccccc5)cc4)nc(-c4ccc5ccc(-c6ccc7ccccc7c6)cc5c4)n3)cc2)cc1 ; c1ccc(-c2nc(-c3ccc4ccc(-c5ccc6ccccc6c5)cc4c3)nc(-c3cccc4oc5ccccc5c34)n2)cc1]

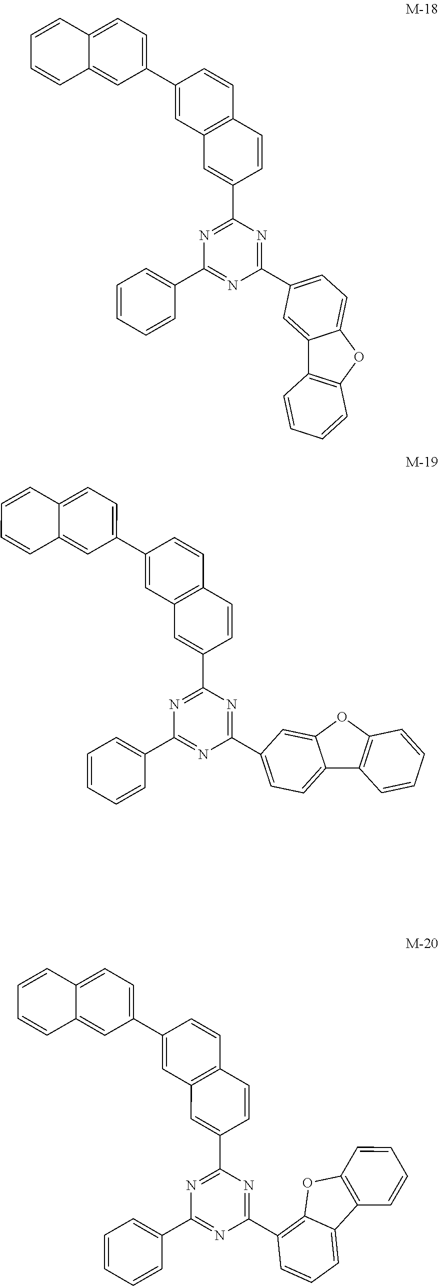 [SMILES: c1ccc(-c2nc(-c3ccc4ccc(-c5ccc6ccccc6c5)cc4c3)nc(-c3ccc4oc5ccccc5c4c3)n2)cc1 ; c1ccc(-c2nc(-c3ccc4ccc(-c5ccc6ccccc6c5)cc4c3)nc(-c3ccc4c(c3)oc3ccccc34)n2)cc1 ; c1ccc(-c2nc(-c3ccc4ccc(-c5ccc6ccccc6c5)cc4c3)nc(-c3cccc4c3oc3ccccc34)n2)cc1]

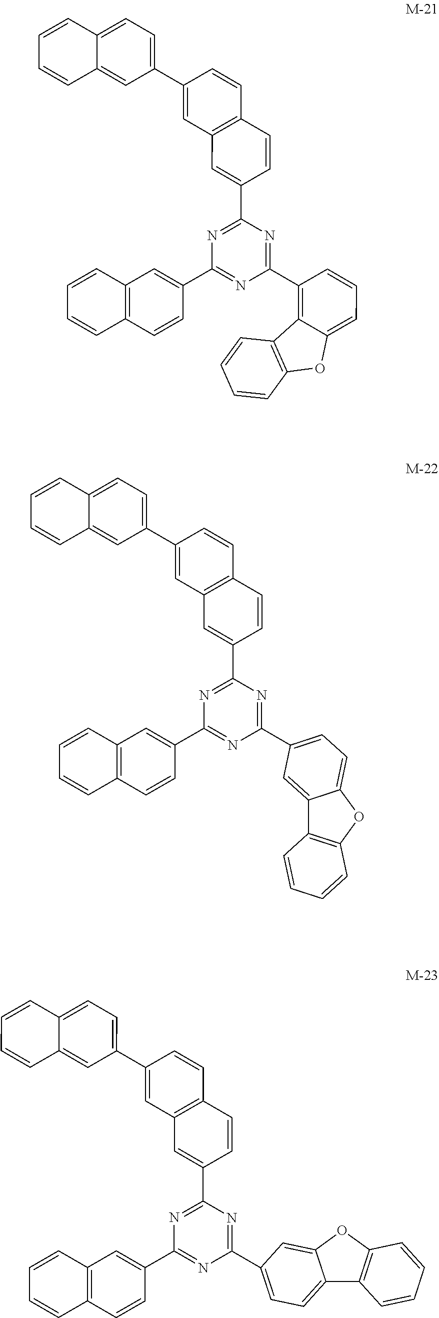 [SMILES: c1ccc2cc(-c3ccc4ccc(-c5nc(-c6ccc7ccccc7c6)nc(-c6cccc7oc8ccccc8c67)n5)cc4c3)ccc2c1 ; c1ccc2cc(-c3ccc4ccc(-c5nc(-c6ccc7ccccc7c6)nc(-c6ccc7oc8ccccc8c7c6)n5)cc4c3)ccc2c1 ; c1ccc2cc(-c3ccc4ccc(-c5nc(-c6ccc7ccccc7c6)nc(-c6ccc7c(c6)oc6ccccc67)n5)cc4c3)ccc2c1]

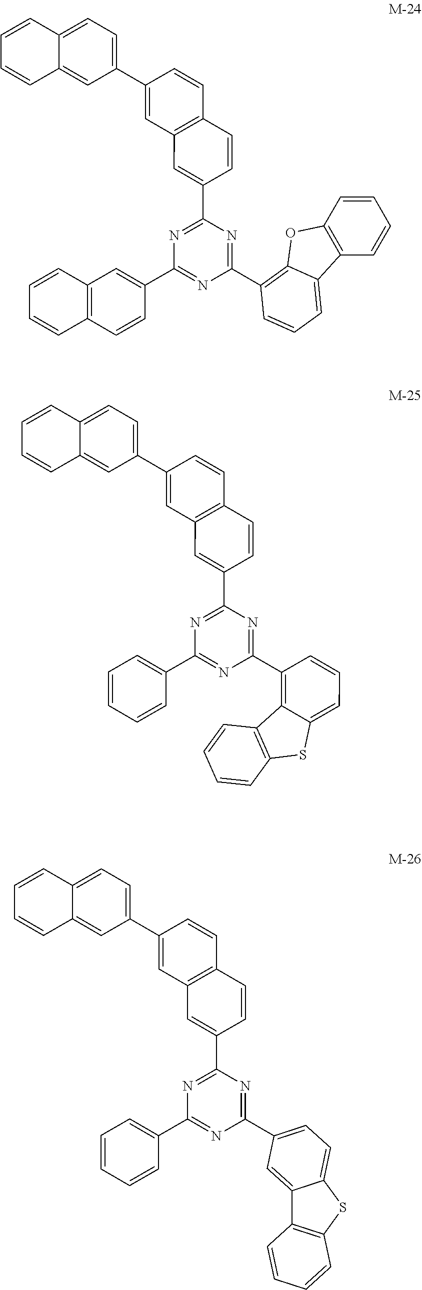 [SMILES: c1ccc2cc(-c3ccc4ccc(-c5nc(-c6ccc7ccccc7c6)nc(-c6cccc7c6oc6ccccc67)n5)cc4c3)ccc2c1 ; c1ccc(-c2nc(-c3ccc4ccc(-c5ccc6ccccc6c5)cc4c3)nc(-c3cccc4sc5ccccc5c34)n2)cc1 ; c1ccc(-c2nc(-c3ccc4ccc(-c5ccc6ccccc6c5)cc4c3)nc(-c3ccc4sc5ccccc5c4c3)n2)cc1]

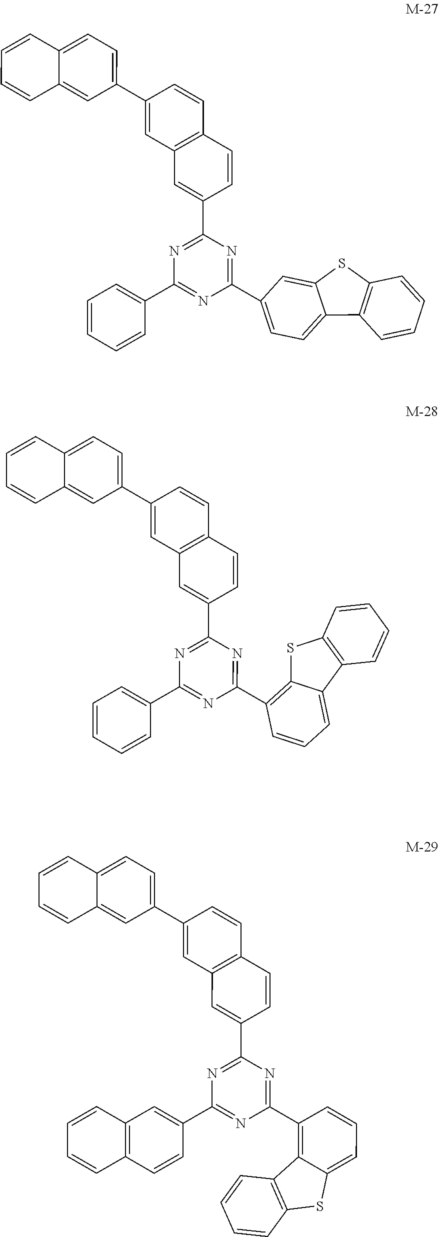 [SMILES: c1ccc(-c2nc(-c3ccc4ccc(-c5ccc6ccccc6c5)cc4c3)nc(-c3ccc4c(c3)sc3ccccc34)n2)cc1 ; c1ccc(-c2nc(-c3ccc4ccc(-c5ccc6ccccc6c5)cc4c3)nc(-c3cccc4c3sc3ccccc34)n2)cc1 ; c1ccc2cc(-c3ccc4ccc(-c5nc(-c6ccc7ccccc7c6)nc(-c6cccc7sc8ccccc8c67)n5)cc4c3)ccc2c1]

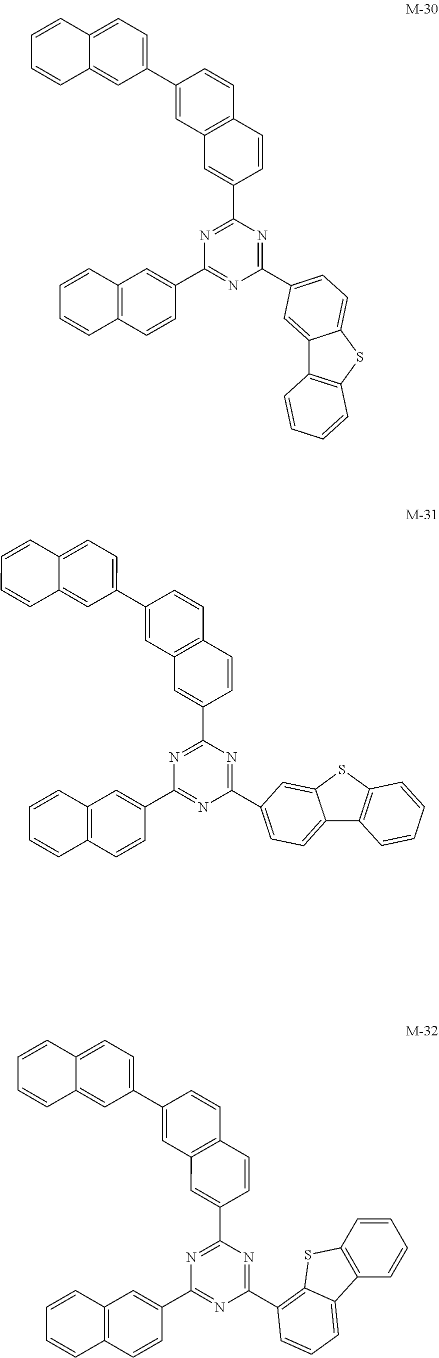 [SMILES: c1ccc2cc(-c3ccc4ccc(-c5nc(-c6ccc7ccccc7c6)nc(-c6ccc7sc8ccccc8c7c6)n5)cc4c3)ccc2c1 ; c1ccc2cc(-c3ccc4ccc(-c5nc(-c6ccc7ccccc7c6)nc(-c6ccc7c(c6)sc6ccccc67)n5)cc4c3)ccc2c1 ; c1ccc2cc(-c3ccc4ccc(-c5nc(-c6ccc7ccccc7c6)nc(-c6cccc7c6sc6ccccc67)n5)cc4c3)ccc2c1]

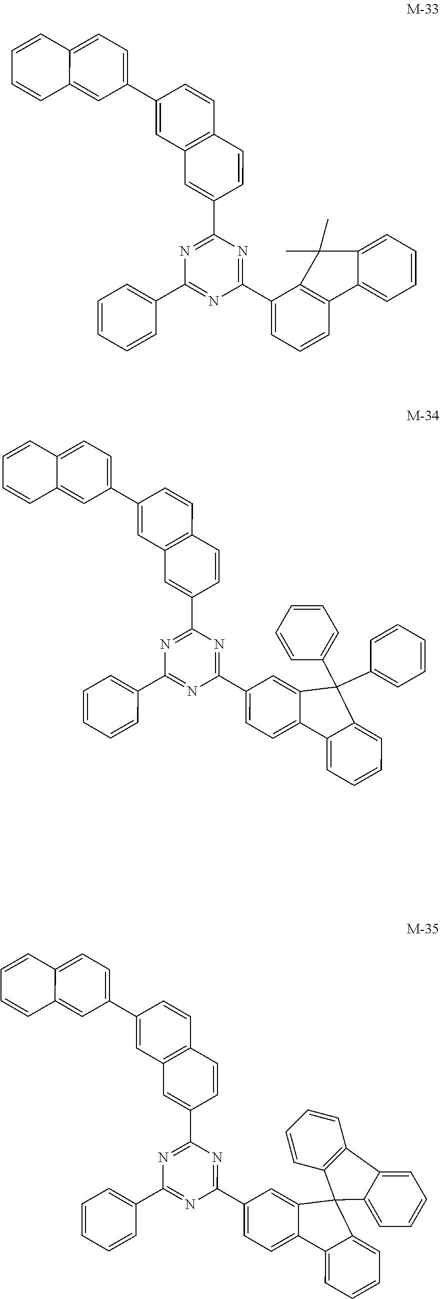 [SMILES: CC1(C)c2ccccc2-c2cccc(-c3nc(-c4ccccc4)nc(-c4ccc5ccc(-c6ccc7ccccc7c6)cc5c4)n3)c21 ; c1ccc(-c2nc(-c3ccc4c(c3)C(c3ccccc3)(c3ccccc3)c3ccccc3-4)nc(-c3ccc4ccc(-c5ccc6ccccc6c5)cc4c3)n2)cc1 ; c1ccc(-c2nc(-c3ccc4c(c3)C3(c5ccccc5-c5ccccc53)c3ccccc3-4)nc(-c3ccc4ccc(-c5ccc6ccccc6c5)cc4c3)n2)cc1]

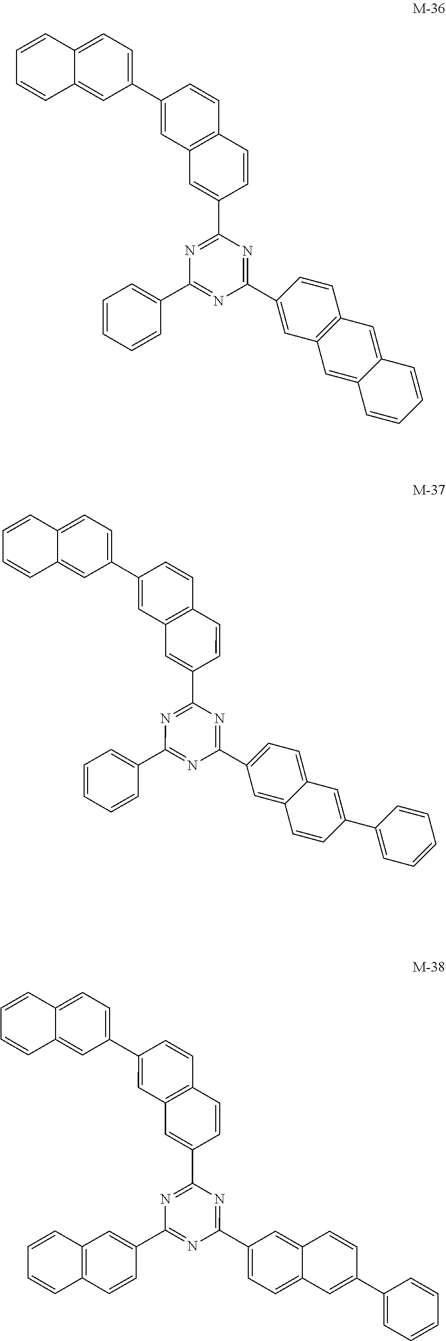 [SMILES: c1ccc(-c2nc(-c3ccc4ccc(-c5ccc6ccccc6c5)cc4c3)nc(-c3ccc4cc5ccccc5cc4c3)n2)cc1 ; c1ccc(-c2ccc3cc(-c4nc(-c5ccccc5)nc(-c5ccc6ccc(-c7ccc8ccccc8c7)cc6c5)n4)ccc3c2)cc1 ; c1ccc(-c2ccc3cc(-c4nc(-c5ccc6ccccc6c5)nc(-c5ccc6ccc(-c7ccc8ccccc8c7)cc6c5)n4)ccc3c2)cc1]

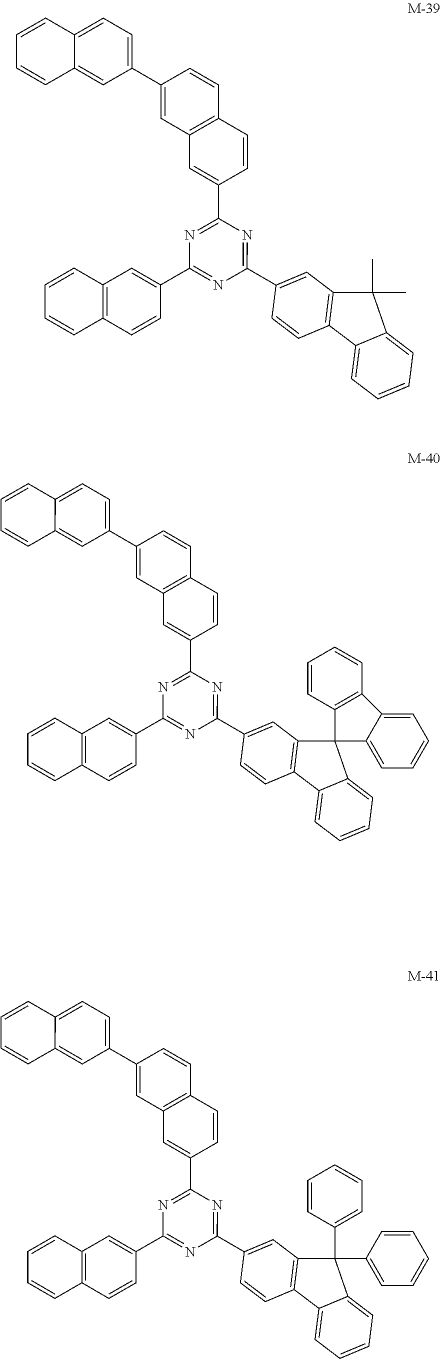 [SMILES: CC1(C)c2ccccc2-c2ccc(-c3nc(-c4ccc5ccccc5c4)nc(-c4ccc5ccc(-c6ccc7ccccc7c6)cc5c4)n3)cc21 ; c1ccc2c(c1)-c1ccccc1C21c2ccccc2-c2ccc(-c3nc(-c4ccc5ccccc5c4)nc(-c4ccc5ccc(-c6ccc7ccccc7c6)cc5c4)n3)cc21 ; c1ccc(C2(c3ccccc3)c3ccccc3-c3ccc(-c4nc(-c5ccc6ccccc6c5)nc(-c5ccc6ccc(-c7ccc8ccccc8c7)cc6c5)n4)cc32)cc1]

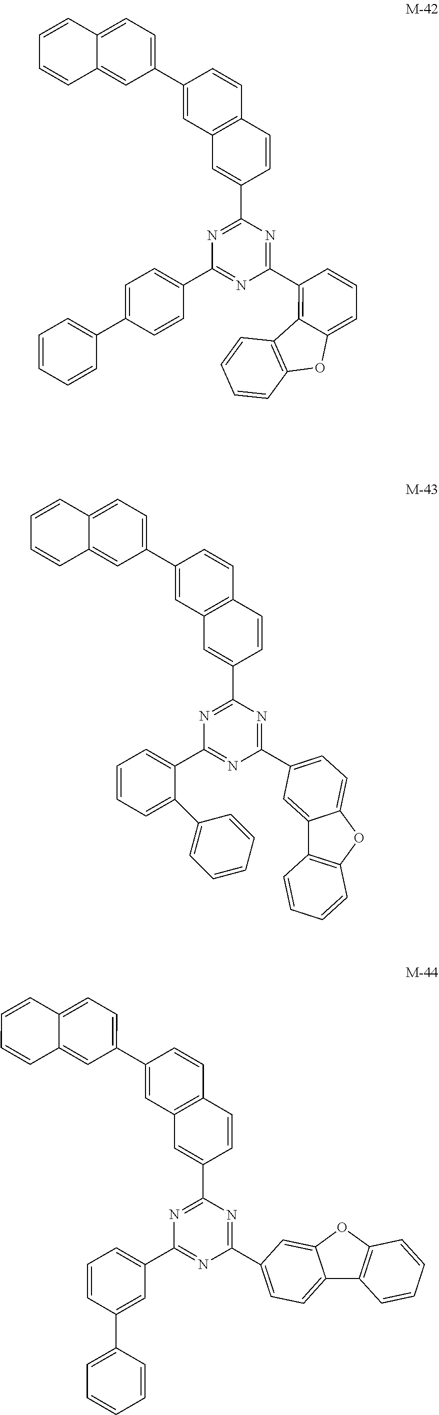 [SMILES: c1ccc(-c2ccc(-c3nc(-c4ccc5ccc(-c6ccc7ccccc7c6)cc5c4)nc(-c4cccc5oc6ccccc6c45)n3)cc2)cc1 ; c1ccc(-c2ccccc2-c2nc(-c3ccc4ccc(-c5ccc6ccccc6c5)cc4c3)nc(-c3ccc4oc5ccccc5c4c3)n2)cc1 ; c1ccc(-c2cccc(-c3nc(-c4ccc5ccc(-c6ccc7ccccc7c6)cc5c4)nc(-c4ccc5c(c4)oc4ccccc45)n3)c2)cc1]

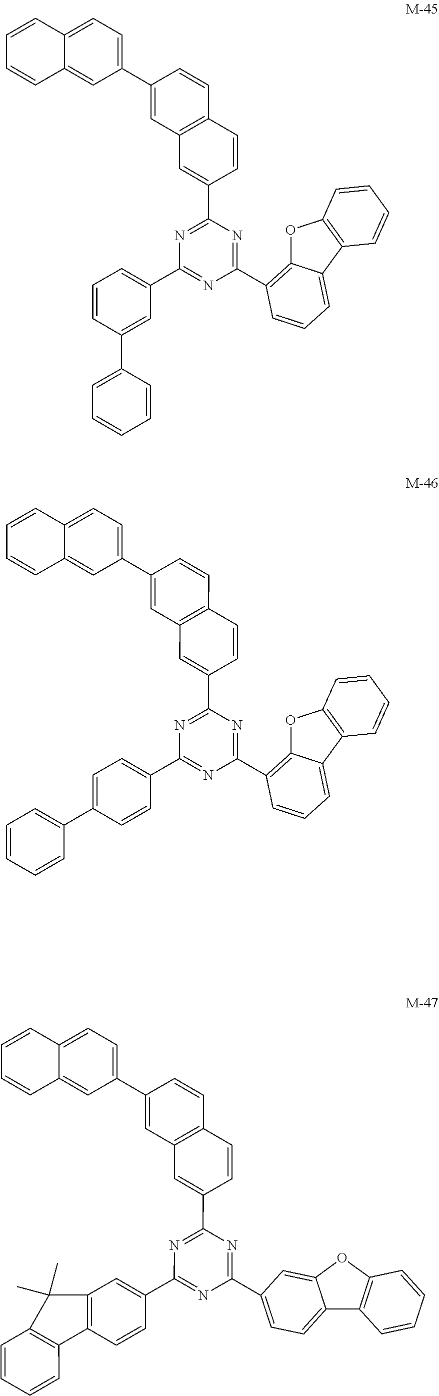 [SMILES: c1ccc(-c2cccc(-c3nc(-c4ccc5ccc(-c6ccc7ccccc7c6)cc5c4)nc(-c4cccc5c4oc4ccccc45)n3)c2)cc1 ; c1ccc(-c2ccc(-c3nc(-c4ccc5ccc(-c6ccc7ccccc7c6)cc5c4)nc(-c4cccc5c4oc4ccccc45)n3)cc2)cc1 ; CC1(C)c2ccccc2-c2ccc(-c3nc(-c4ccc5ccc(-c6ccc7ccccc7c6)cc5c4)nc(-c4ccc5c(c4)oc4ccccc45)n3)cc21]

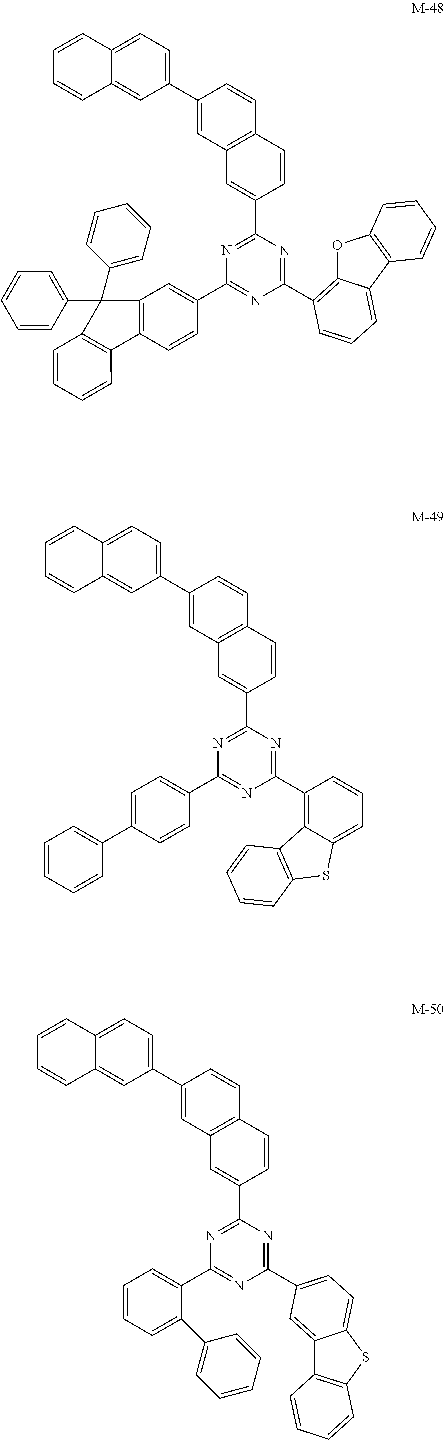 [SMILES: c1ccc(C2(c3ccccc3)c3ccccc3-c3ccc(-c4nc(-c5ccc6ccc(-c7ccc8ccccc8c7)cc6c5)nc(-c5cccc6c5oc5ccccc56)n4)cc32)cc1 ; c1ccc(-c2ccc(-c3nc(-c4ccc5ccc(-c6ccc7ccccc7c6)cc5c4)nc(-c4cccc5sc6ccccc6c45)n3)cc2)cc1 ; c1ccc(-c2ccccc2-c2nc(-c3ccc4ccc(-c5ccc6ccccc6c5)cc4c3)nc(-c3ccc4sc5ccccc5c4c3)n2)cc1]

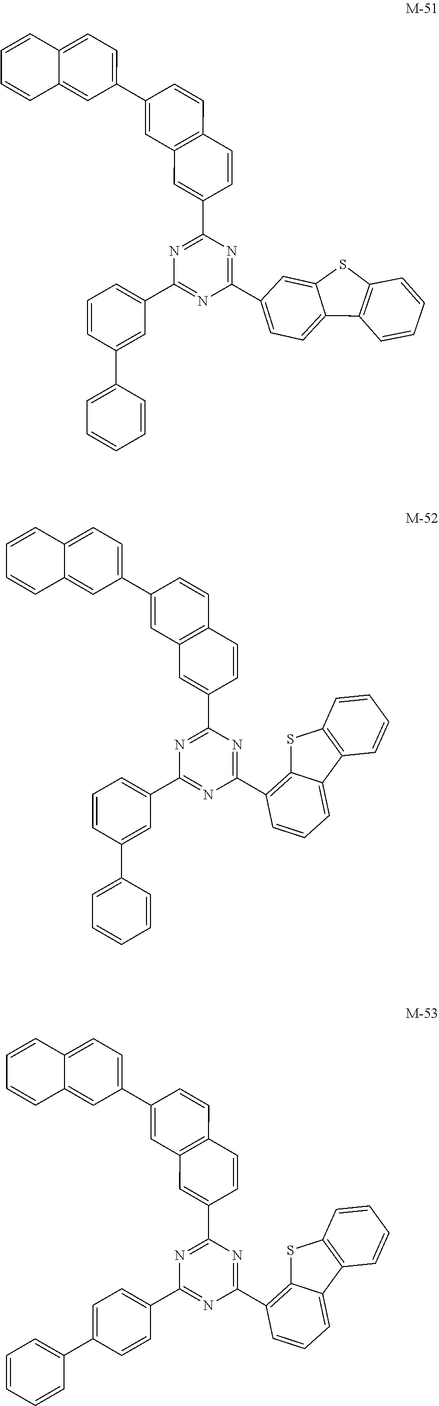 [SMILES: c1ccc(-c2cccc(-c3nc(-c4ccc5ccc(-c6ccc7ccccc7c6)cc5c4)nc(-c4ccc5c(c4)sc4ccccc45)n3)c2)cc1 ; c1ccc(-c2cccc(-c3nc(-c4ccc5ccc(-c6ccc7ccccc7c6)cc5c4)nc(-c4cccc5c4sc4ccccc45)n3)c2)cc1 ; c1ccc(-c2ccc(-c3nc(-c4ccc5ccc(-c6ccc7ccccc7c6)cc5c4)nc(-c4cccc5c4sc4ccccc45)n3)cc2)cc1]

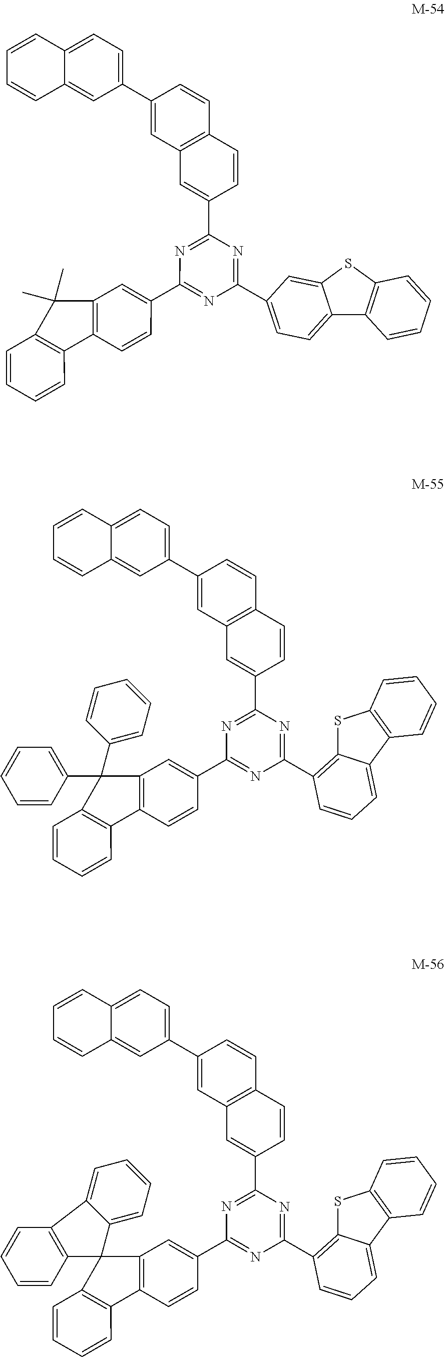 [SMILES: CC1(C)c2ccccc2-c2ccc(-c3nc(-c4ccc5ccc(-c6ccc7ccccc7c6)cc5c4)nc(-c4ccc5c(c4)sc4ccccc45)n3)cc21 ; c1ccc(C2(c3ccccc3)c3ccccc3-c3ccc(-c4nc(-c5ccc6ccc(-c7ccc8ccccc8c7)cc6c5)nc(-c5cccc6c5sc5ccccc56)n4)cc32)cc1 ; c1ccc2c(c1)-c1ccccc1C21c2ccccc2-c2ccc(-c3nc(-c4ccc5ccc(-c6ccc7ccccc7c6)cc5c4)nc(-c4cccc5c4sc4ccccc45)n3)cc21]

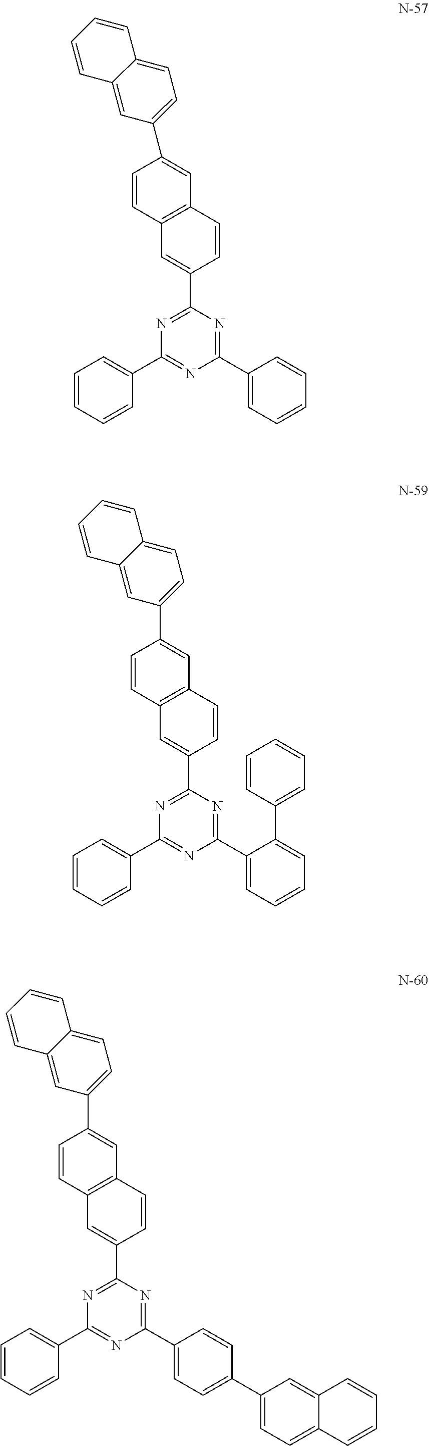 [SMILES: c1ccc(-c2nc(-c3ccccc3)nc(-c3ccc4cc(-c5ccc6ccccc6c5)ccc4c3)n2)cc1 ; c1ccc(-c2nc(-c3ccc4cc(-c5ccc6ccccc6c5)ccc4c3)nc(-c3ccccc3-c3ccccc3)n2)cc1 ; c1ccc(-c2nc(-c3ccc(-c4ccc5ccccc5c4)cc3)nc(-c3ccc4cc(-c5ccc6ccccc6c5)ccc4c3)n2)cc1]

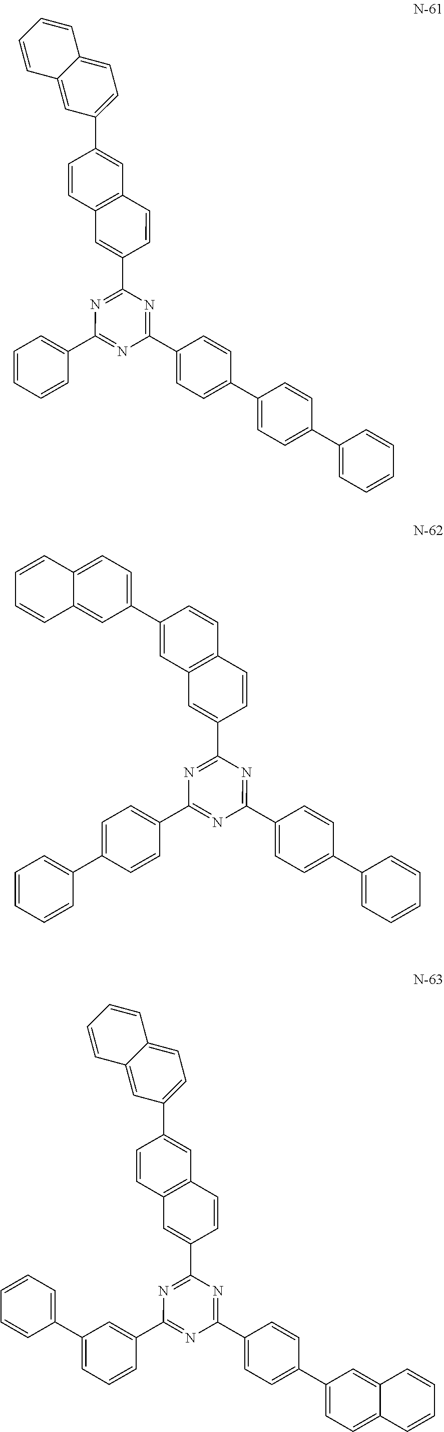 [SMILES: c1ccc(-c2ccc(-c3ccc(-c4nc(-c5ccccc5)nc(-c5ccc6cc(-c7ccc8ccccc8c7)ccc6c5)n4)cc3)cc2)cc1 ; c1ccc(-c2ccc(-c3nc(-c4ccc(-c5ccccc5)cc4)nc(-c4ccc5ccc(-c6ccc7ccccc7c6)cc5c4)n3)cc2)cc1 ; c1ccc(-c2cccc(-c3nc(-c4ccc(-c5ccc6ccccc6c5)cc4)nc(-c4ccc5cc(-c6ccc7ccccc7c6)ccc5c4)n3)c2)cc1]

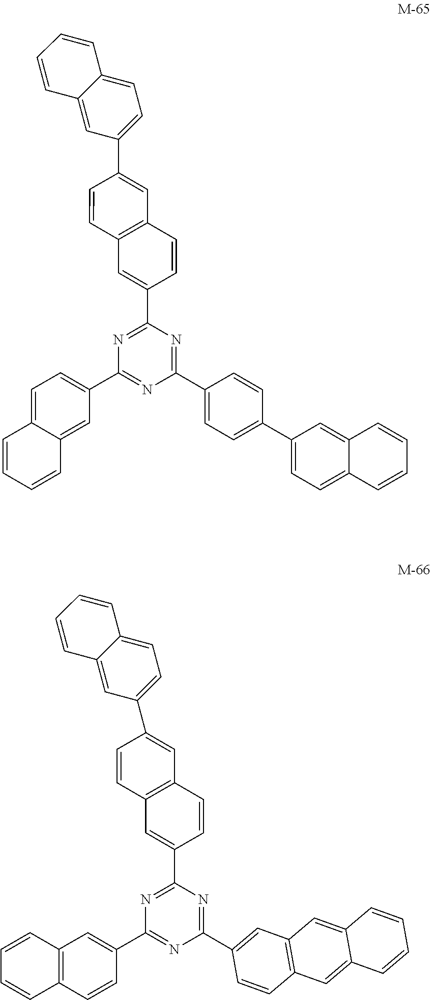 [SMILES: c1ccc2cc(-c3ccc(-c4nc(-c5ccc6ccccc6c5)nc(-c5ccc6cc(-c7ccc8ccccc8c7)ccc6c5)n4)cc3)ccc2c1 ; c1ccc2cc(-c3ccc4cc(-c5nc(-c6ccc7ccccc7c6)nc(-c6ccc7cc8ccccc8cc7c6)n5)ccc4c3)ccc2c1]

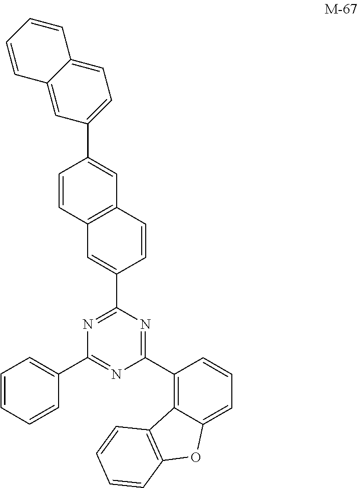 [SMILES: c1ccc(-c2nc(-c3ccc4cc(-c5ccc6ccccc6c5)ccc4c3)nc(-c3cccc4oc5ccccc5c34)n2)cc1]

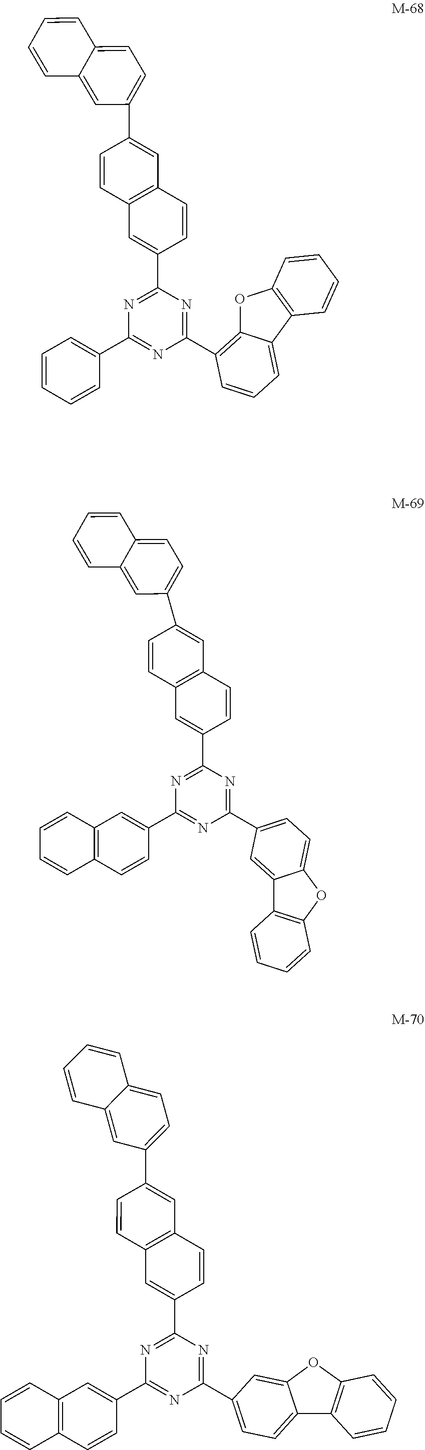 [SMILES: c1ccc(-c2nc(-c3ccc4cc(-c5ccc6ccccc6c5)ccc4c3)nc(-c3cccc4c3oc3ccccc34)n2)cc1 ; c1ccc2cc(-c3ccc4cc(-c5nc(-c6ccc7ccccc7c6)nc(-c6ccc7oc8ccccc8c7c6)n5)ccc4c3)ccc2c1 ; c1ccc2cc(-c3ccc4cc(-c5nc(-c6ccc7ccccc7c6)nc(-c6ccc7c(c6)oc6ccccc67)n5)ccc4c3)ccc2c1]

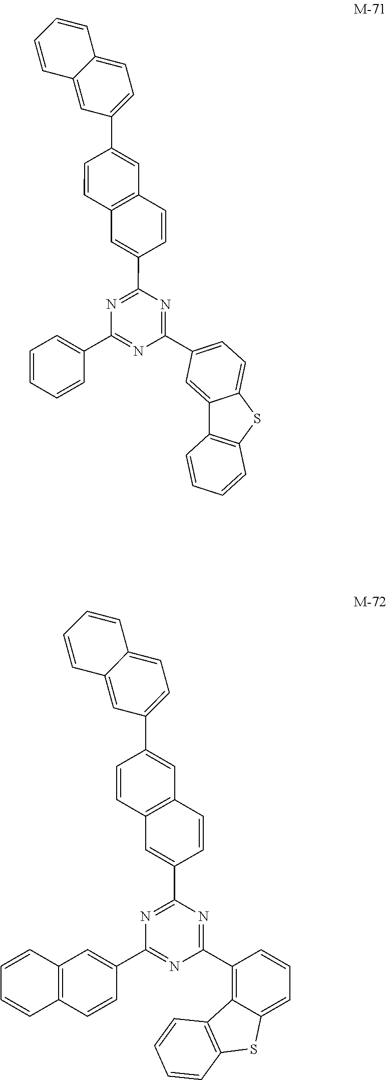 [SMILES: c1ccc(-c2nc(-c3ccc4cc(-c5ccc6ccccc6c5)ccc4c3)nc(-c3ccc4sc5ccccc5c4c3)n2)cc1 ; c1ccc2cc(-c3ccc4cc(-c5nc(-c6ccc7ccccc7c6)nc(-c6cccc7sc8ccccc8c67)n5)ccc4c3)ccc2c1]

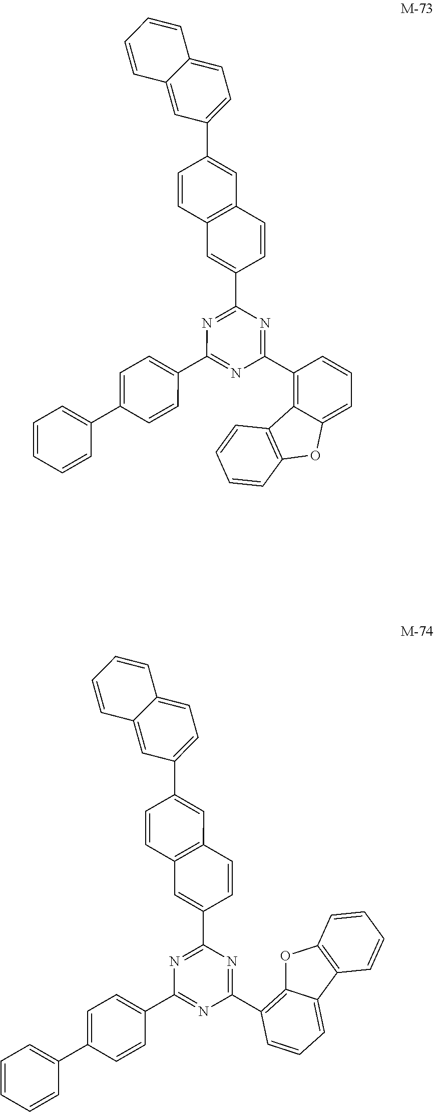 [SMILES: c1ccc(-c2ccc(-c3nc(-c4ccc5cc(-c6ccc7ccccc7c6)ccc5c4)nc(-c4cccc5oc6ccccc6c45)n3)cc2)cc1 ; c1ccc(-c2ccc(-c3nc(-c4ccc5cc(-c6ccc7ccccc7c6)ccc5c4)nc(-c4cccc5c4oc4ccccc45)n3)cc2)cc1]

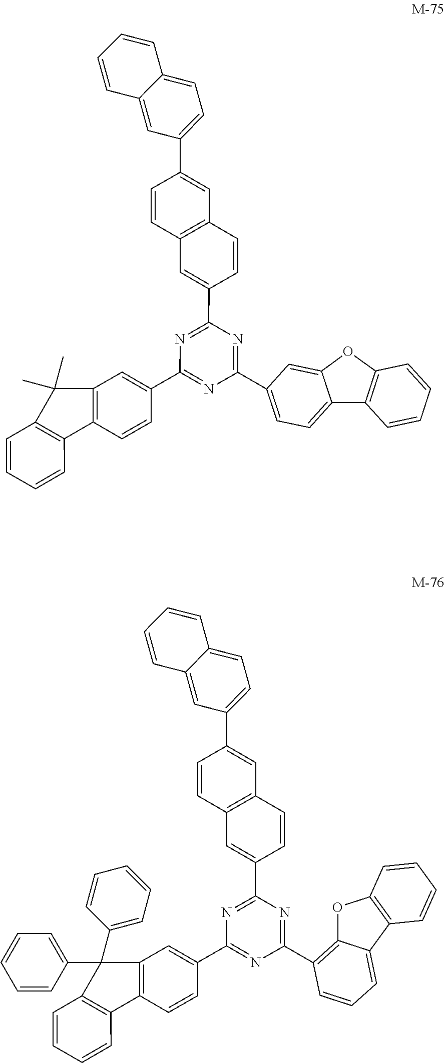 [SMILES: CC1(C)c2ccccc2-c2ccc(-c3nc(-c4ccc5cc(-c6ccc7ccccc7c6)ccc5c4)nc(-c4ccc5c(c4)oc4ccccc45)n3)cc21 ; c1ccc(C2(c3ccccc3)c3ccccc3-c3ccc(-c4nc(-c5ccc6cc(-c7ccc8ccccc8c7)ccc6c5)nc(-c5cccc6c5oc5ccccc56)n4)cc32)cc1]

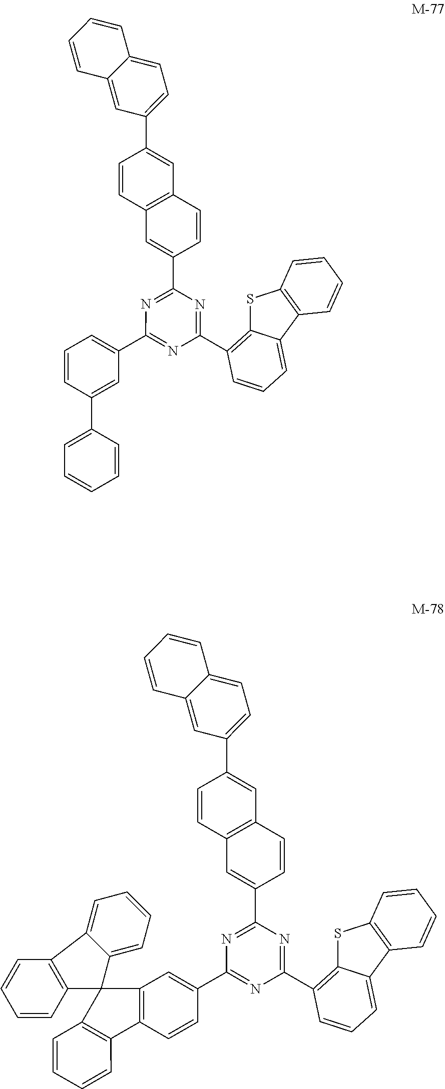 [SMILES: c1ccc(-c2cccc(-c3nc(-c4ccc5cc(-c6ccc7ccccc7c6)ccc5c4)nc(-c4cccc5c4sc4ccccc45)n3)c2)cc1 ; c1ccc2c(c1)-c1ccccc1C21c2ccccc2-c2ccc(-c3nc(-c4ccc5cc(-c6ccc7ccccc7c6)ccc5c4)nc(-c4cccc5c4sc4ccccc45)n3)cc21]

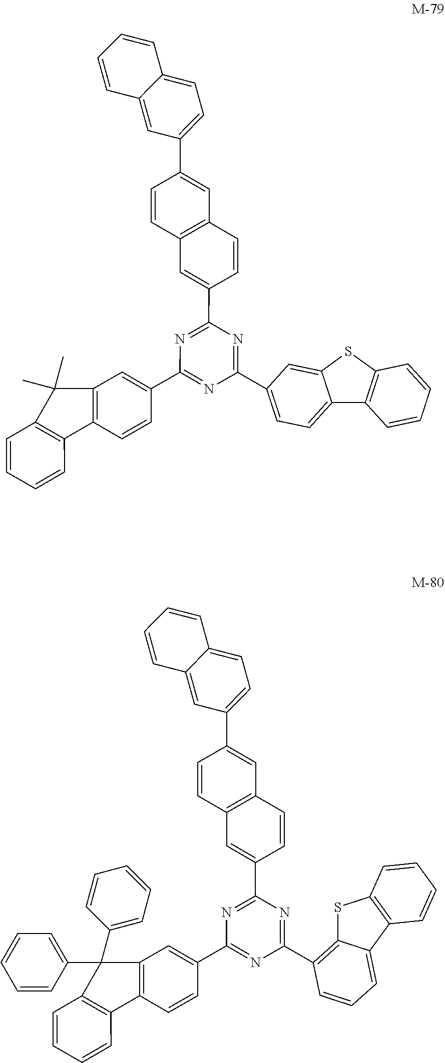 [SMILES: CC1(C)c2ccccc2-c2ccc(-c3nc(-c4ccc5cc(-c6ccc7ccccc7c6)ccc5c4)nc(-c4ccc5c(c4)sc4ccccc45)n3)cc21 ; c1ccc(C2(c3ccccc3)c3ccccc3-c3ccc(-c4nc(-c5ccc6cc(-c7ccc8ccccc8c7)ccc6c5)nc(-c5cccc6c5sc5ccccc56)n4)cc32)cc1]

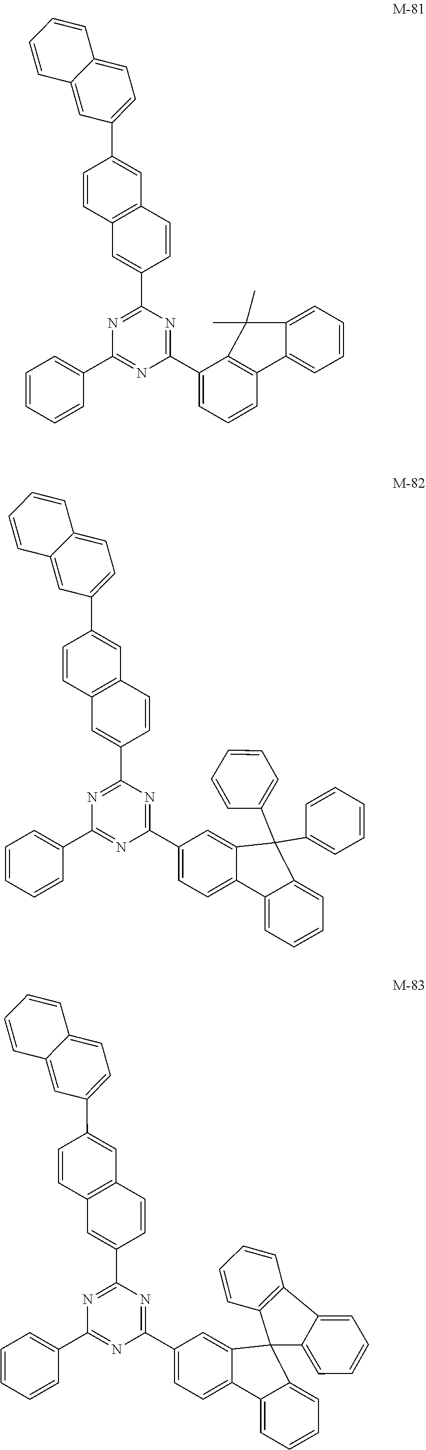 [SMILES: CC1(C)c2ccccc2-c2cccc(-c3nc(-c4ccccc4)nc(-c4ccc5cc(-c6ccc7ccccc7c6)ccc5c4)n3)c21 ; c1ccc(-c2nc(-c3ccc4c(c3)C(c3ccccc3)(c3ccccc3)c3ccccc3-4)nc(-c3ccc4cc(-c5ccc6ccccc6c5)ccc4c3)n2)cc1 ; c1ccc(-c2nc(-c3ccc4c(c3)C3(c5ccccc5-c5ccccc53)c3ccccc3-4)nc(-c3ccc4cc(-c5ccc6ccccc6c5)ccc4c3)n2)cc1]

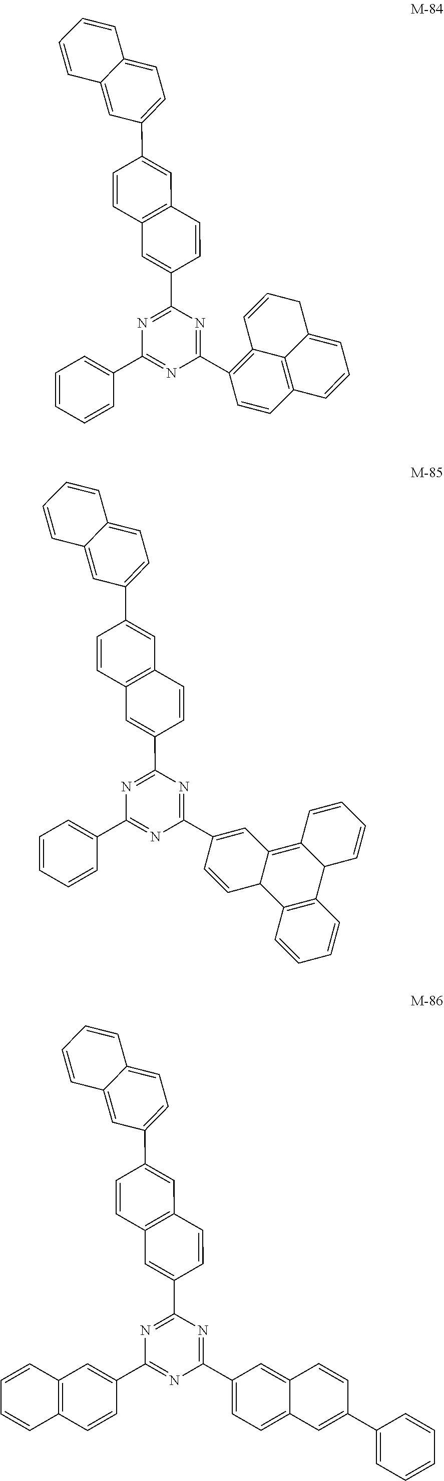 [SMILES: C1=Cc2c(-c3nc(-c4ccccc4)nc(-c4ccc5cc(-c6ccc7ccccc7c6)ccc5c4)n3)ccc3cccc(c23)C1 ; C1=CC2=C3C=C(c4nc(-c5ccccc5)nc(-c5ccc6cc(-c7ccc8ccccc8c7)ccc6c5)n4)C=CC3c3ccccc3C2C=C1 ; c1ccc(-c2ccc3cc(-c4nc(-c5ccc6ccccc6c5)nc(-c5ccc6cc(-c7ccc8ccccc8c7)ccc6c5)n4)ccc3c2)cc1]

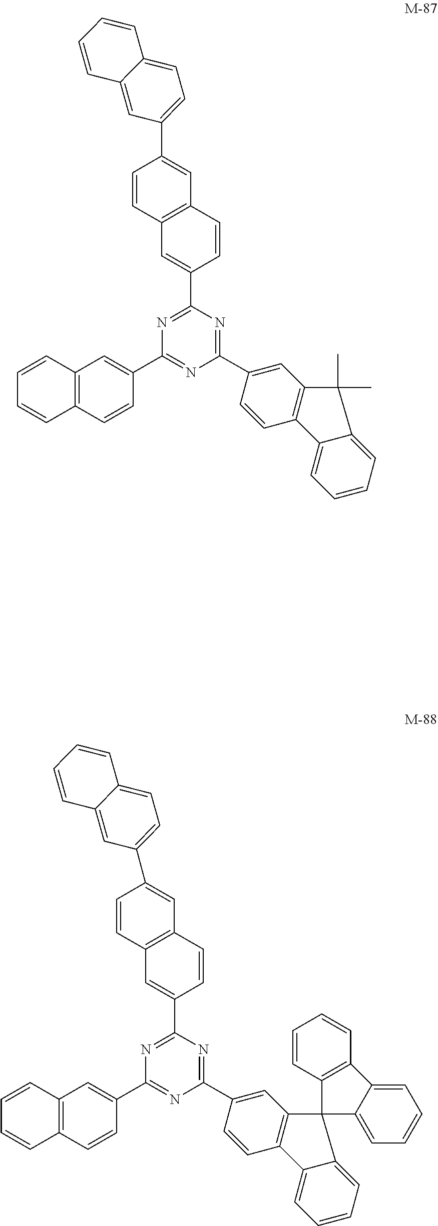 [SMILES: CC1(C)c2ccccc2-c2ccc(-c3nc(-c4ccc5ccccc5c4)nc(-c4ccc5cc(-c6ccc7ccccc7c6)ccc5c4)n3)cc21 ; c1ccc2c(c1)-c1ccccc1C21c2ccccc2-c2ccc(-c3nc(-c4ccc5ccccc5c4)nc(-c4ccc5cc(-c6ccc7ccccc7c6)ccc5c4)n3)cc21]

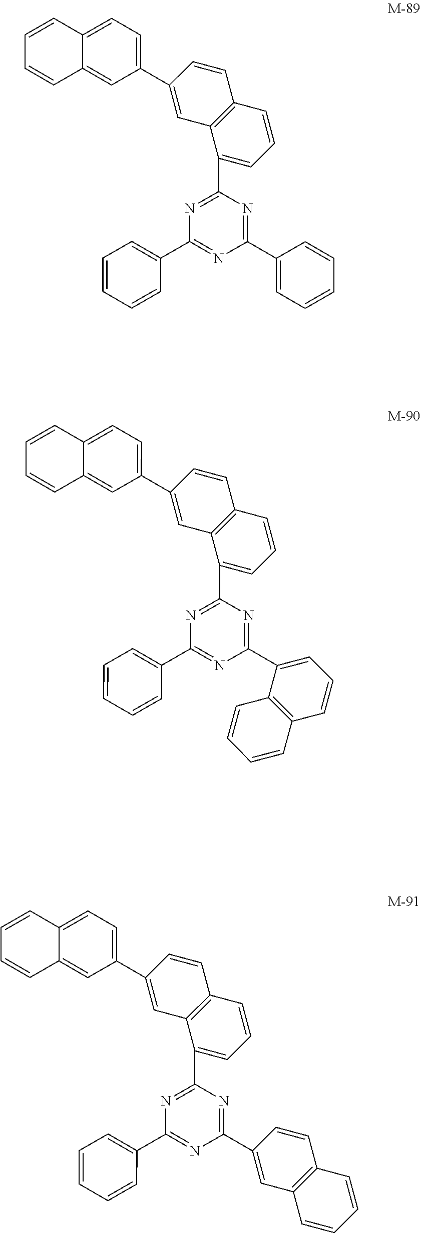 [SMILES: c1ccc(-c2nc(-c3ccccc3)nc(-c3cccc4ccc(-c5ccc6ccccc6c5)cc34)n2)cc1 ; c1ccc(-c2nc(-c3cccc4ccccc34)nc(-c3cccc4ccc(-c5ccc6ccccc6c5)cc34)n2)cc1 ; c1ccc(-c2nc(-c3ccc4ccccc4c3)nc(-c3cccc4ccc(-c5ccc6ccccc6c5)cc34)n2)cc1]

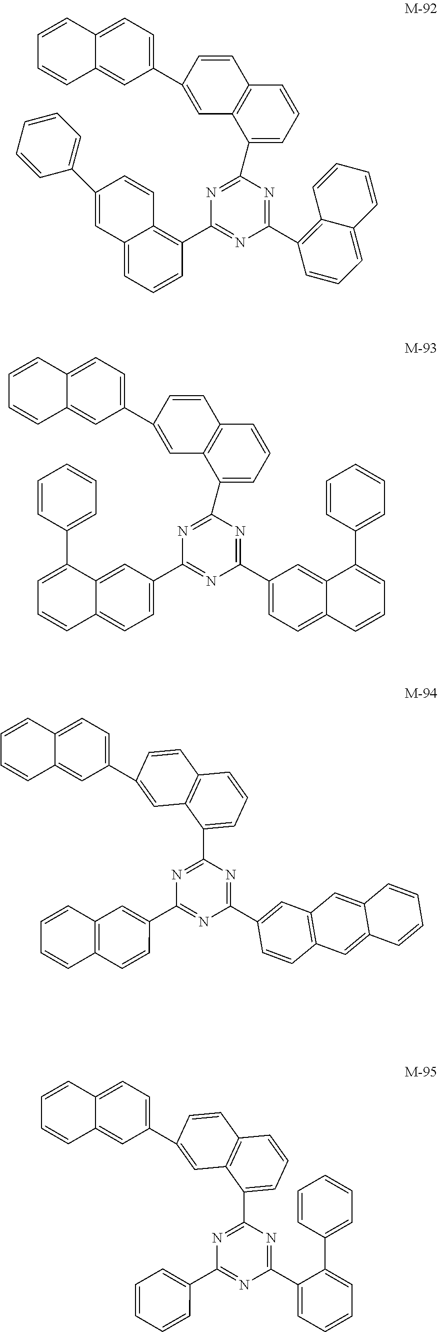 [SMILES: c1ccc(-c2ccc3c(-c4nc(-c5cccc6ccccc56)nc(-c5cccc6ccc(-c7ccc8ccccc8c7)cc56)n4)cccc3c2)cc1 ; c1ccc(-c2cccc3ccc(-c4nc(-c5ccc6cccc(-c7ccccc7)c6c5)nc(-c5cccc6ccc(-c7ccc8ccccc8c7)cc56)n4)cc23)cc1 ; c1ccc2cc(-c3ccc4cccc(-c5nc(-c6ccc7ccccc7c6)nc(-c6ccc7cc8ccccc8cc7c6)n5)c4c3)ccc2c1 ; c1ccc(-c2nc(-c3ccccc3-c3ccccc3)nc(-c3cccc4ccc(-c5ccc6ccccc6c5)cc34)n2)cc1]

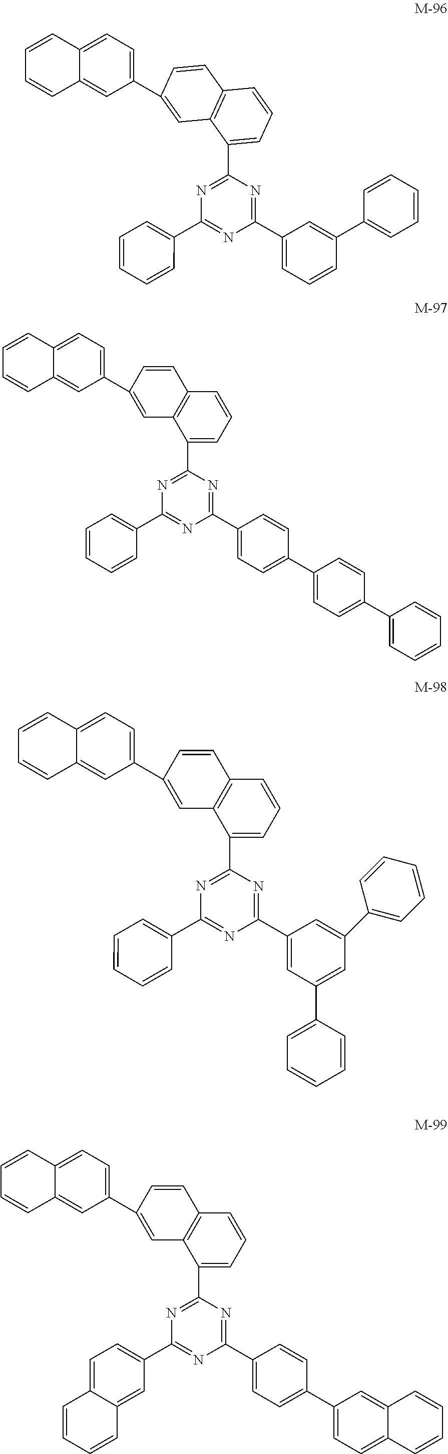 [SMILES: c1ccc(-c2cccc(-c3nc(-c4ccccc4)nc(-c4cccc5ccc(-c6ccc7ccccc7c6)cc45)n3)c2)cc1 ; c1ccc(-c2ccc(-c3ccc(-c4nc(-c5ccccc5)nc(-c5cccc6ccc(-c7ccc8ccccc8c7)cc56)n4)cc3)cc2)cc1 ; c1ccc(-c2cc(-c3ccccc3)cc(-c3nc(-c4ccccc4)nc(-c4cccc5ccc(-c6ccc7ccccc7c6)cc45)n3)c2)cc1 ; c1ccc2cc(-c3ccc(-c4nc(-c5ccc6ccccc6c5)nc(-c5cccc6ccc(-c7ccc8ccccc8c7)cc56)n4)cc3)ccc2c1]

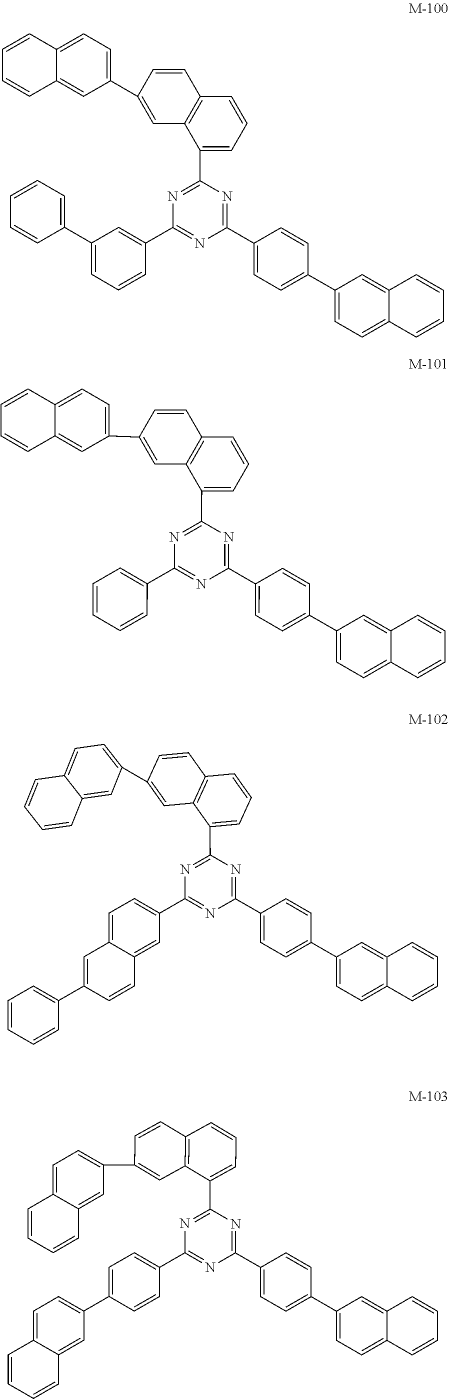 [SMILES: c1ccc(-c2cccc(-c3nc(-c4ccc(-c5ccc6ccccc6c5)cc4)nc(-c4cccc5ccc(-c6ccc7ccccc7c6)cc45)n3)c2)cc1 ; c1ccc(-c2nc(-c3ccc(-c4ccc5ccccc5c4)cc3)nc(-c3cccc4ccc(-c5ccc6ccccc6c5)cc34)n2)cc1 ; c1ccc(-c2ccc3cc(-c4nc(-c5ccc(-c6ccc7ccccc7c6)cc5)nc(-c5cccc6ccc(-c7ccc8ccccc8c7)cc56)n4)ccc3c2)cc1 ; c1ccc2cc(-c3ccc(-c4nc(-c5ccc(-c6ccc7ccccc7c6)cc5)nc(-c5cccc6ccc(-c7ccc8ccccc8c7)cc56)n4)cc3)ccc2c1]

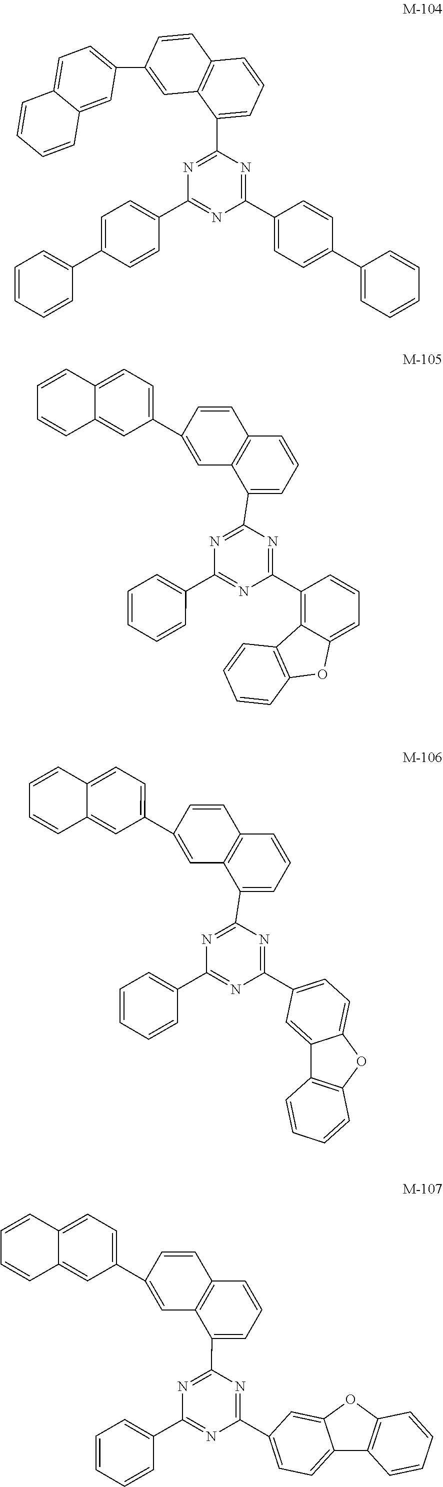 [SMILES: c1ccc(-c2ccc(-c3nc(-c4ccc(-c5ccccc5)cc4)nc(-c4cccc5ccc(-c6ccc7ccccc7c6)cc45)n3)cc2)cc1 ; c1ccc(-c2nc(-c3cccc4ccc(-c5ccc6ccccc6c5)cc34)nc(-c3cccc4oc5ccccc5c34)n2)cc1 ; c1ccc(-c2nc(-c3ccc4oc5ccccc5c4c3)nc(-c3cccc4ccc(-c5ccc6ccccc6c5)cc34)n2)cc1 ; c1ccc(-c2nc(-c3ccc4c(c3)oc3ccccc34)nc(-c3cccc4ccc(-c5ccc6ccccc6c5)cc34)n2)cc1]

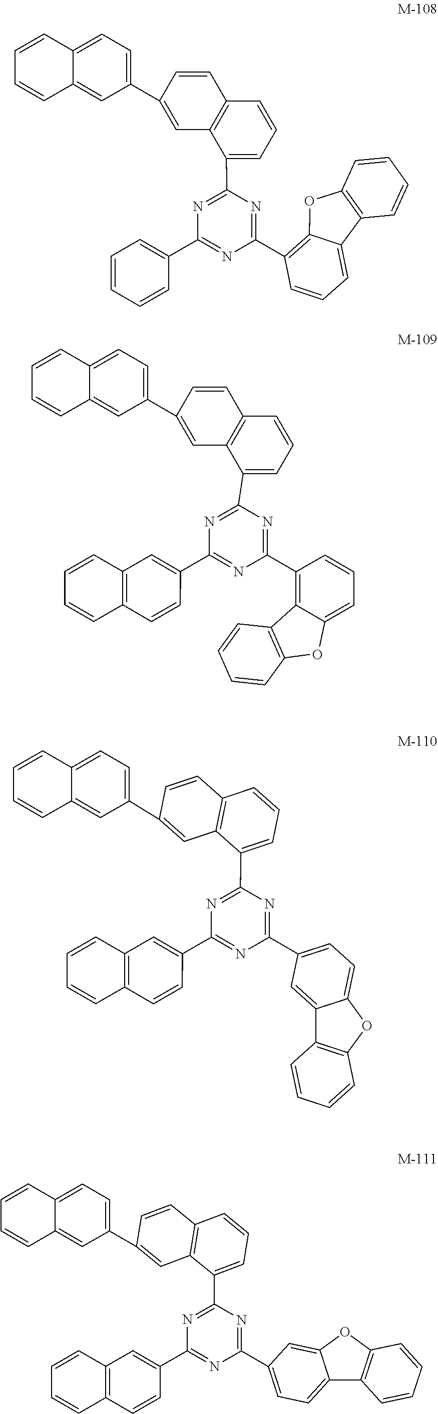 [SMILES: c1ccc(-c2nc(-c3cccc4ccc(-c5ccc6ccccc6c5)cc34)nc(-c3cccc4c3oc3ccccc34)n2)cc1 ; c1ccc2cc(-c3ccc4cccc(-c5nc(-c6ccc7ccccc7c6)nc(-c6cccc7oc8ccccc8c67)n5)c4c3)ccc2c1 ; c1ccc2cc(-c3ccc4cccc(-c5nc(-c6ccc7ccccc7c6)nc(-c6ccc7oc8ccccc8c7c6)n5)c4c3)ccc2c1 ; c1ccc2cc(-c3ccc4cccc(-c5nc(-c6ccc7ccccc7c6)nc(-c6ccc7c(c6)oc6ccccc67)n5)c4c3)ccc2c1]

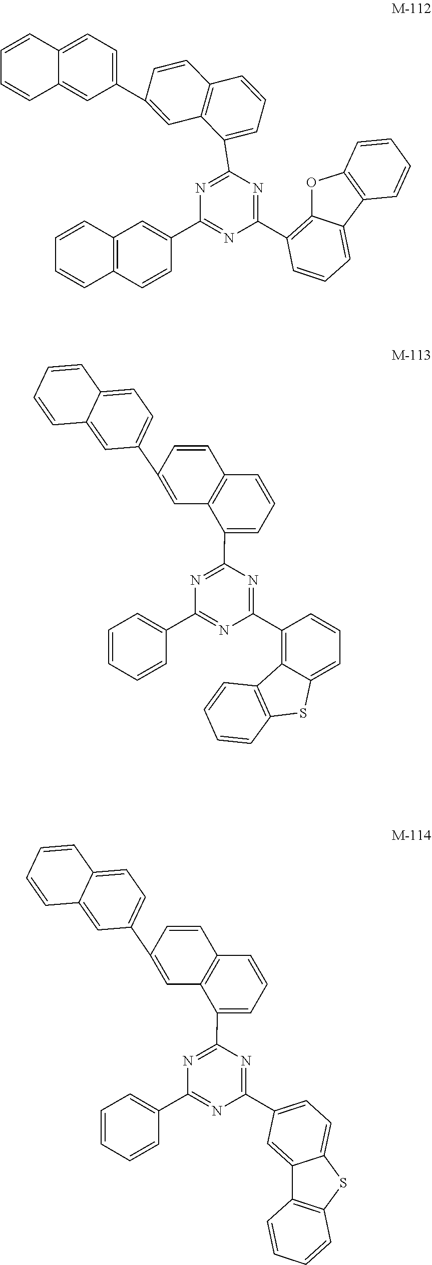 [SMILES: c1ccc2cc(-c3ccc4cccc(-c5nc(-c6ccc7ccccc7c6)nc(-c6cccc7c6oc6ccccc67)n5)c4c3)ccc2c1 ; c1ccc(-c2nc(-c3cccc4ccc(-c5ccc6ccccc6c5)cc34)nc(-c3cccc4sc5ccccc5c34)n2)cc1 ; c1ccc(-c2nc(-c3ccc4sc5ccccc5c4c3)nc(-c3cccc4ccc(-c5ccc6ccccc6c5)cc34)n2)cc1]

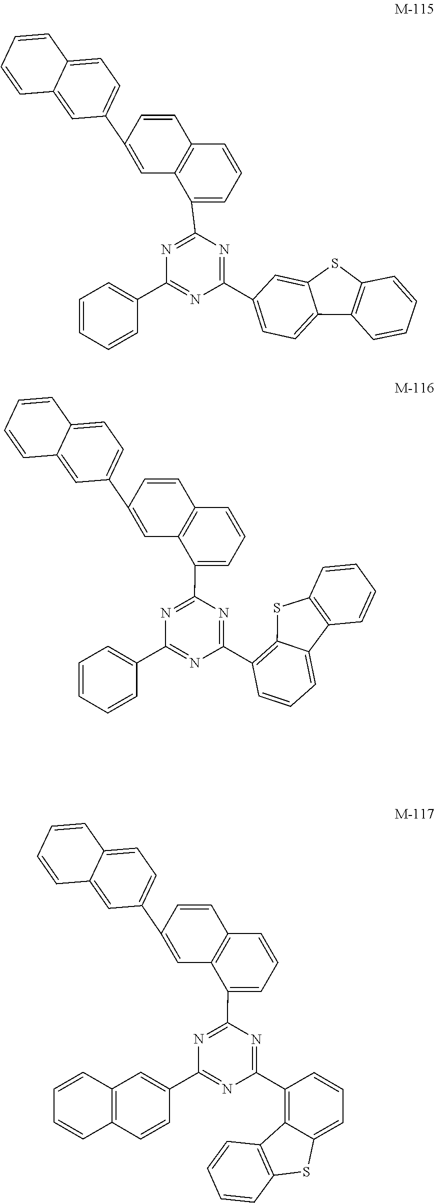 [SMILES: c1ccc(-c2nc(-c3ccc4c(c3)sc3ccccc34)nc(-c3cccc4ccc(-c5ccc6ccccc6c5)cc34)n2)cc1 ; c1ccc(-c2nc(-c3cccc4ccc(-c5ccc6ccccc6c5)cc34)nc(-c3cccc4c3sc3ccccc34)n2)cc1 ; c1ccc2cc(-c3ccc4cccc(-c5nc(-c6ccc7ccccc7c6)nc(-c6cccc7sc8ccccc8c67)n5)c4c3)ccc2c1]

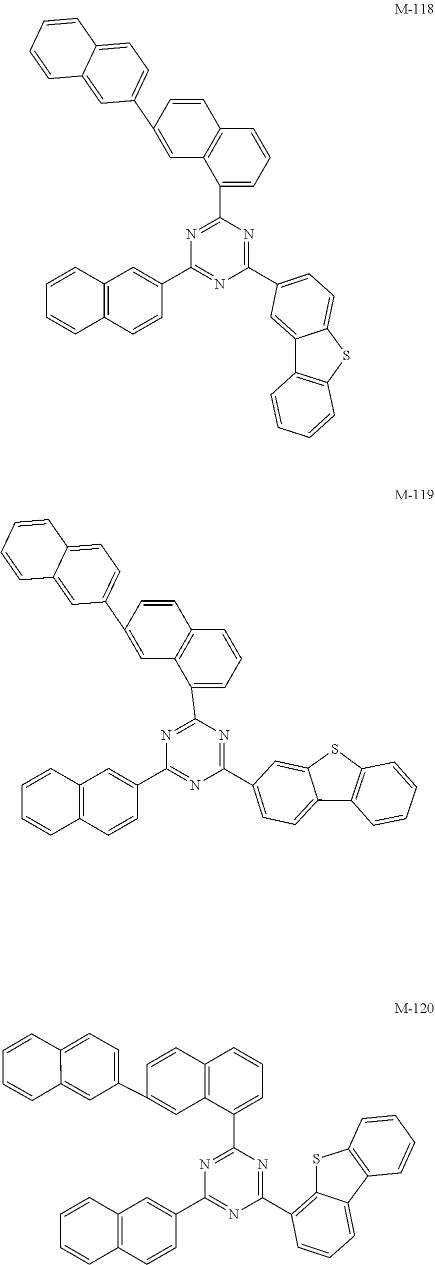 [SMILES: c1ccc2cc(-c3ccc4cccc(-c5nc(-c6ccc7ccccc7c6)nc(-c6ccc7sc8ccccc8c7c6)n5)c4c3)ccc2c1 ; c1ccc2cc(-c3ccc4cccc(-c5nc(-c6ccc7ccccc7c6)nc(-c6ccc7c(c6)sc6ccccc67)n5)c4c3)ccc2c1 ; c1ccc2cc(-c3ccc4cccc(-c5nc(-c6ccc7ccccc7c6)nc(-c6cccc7c6sc6ccccc67)n5)c4c3)ccc2c1]

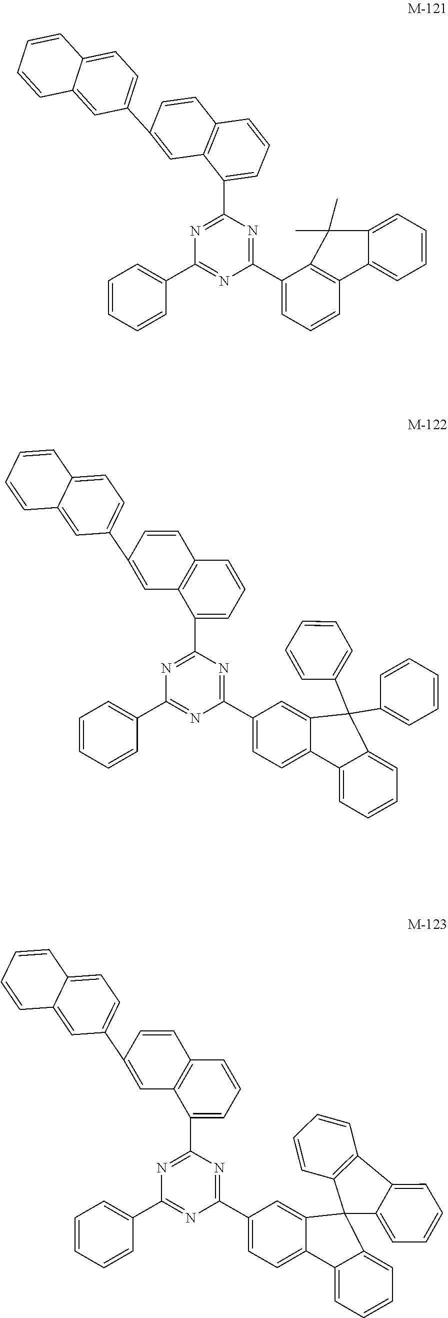 [SMILES: CC1(C)c2ccccc2-c2cccc(-c3nc(-c4ccccc4)nc(-c4cccc5ccc(-c6ccc7ccccc7c6)cc45)n3)c21 ; c1ccc(-c2nc(-c3ccc4c(c3)C(c3ccccc3)(c3ccccc3)c3ccccc3-4)nc(-c3cccc4ccc(-c5ccc6ccccc6c5)cc34)n2)cc1 ; c1ccc(-c2nc(-c3ccc4c(c3)C3(c5ccccc5-c5ccccc53)c3ccccc3-4)nc(-c3cccc4ccc(-c5ccc6ccccc6c5)cc34)n2)cc1]

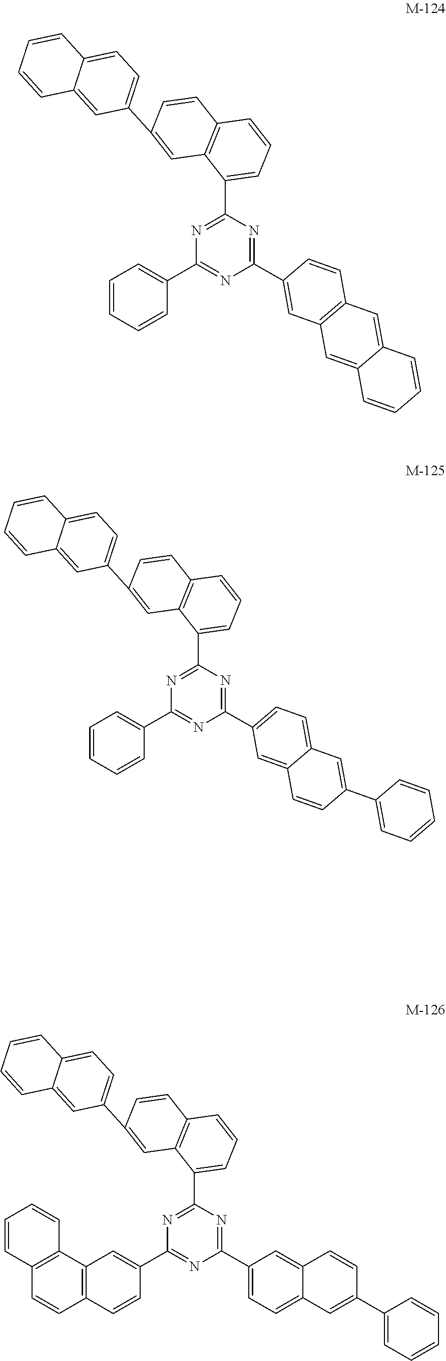 [SMILES: c1ccc(-c2nc(-c3ccc4cc5ccccc5cc4c3)nc(-c3cccc4ccc(-c5ccc6ccccc6c5)cc34)n2)cc1 ; c1ccc(-c2ccc3cc(-c4nc(-c5ccccc5)nc(-c5cccc6ccc(-c7ccc8ccccc8c7)cc56)n4)ccc3c2)cc1 ; c1ccc(-c2ccc3cc(-c4nc(-c5ccc6ccc7ccccc7c6c5)nc(-c5cccc6ccc(-c7ccc8ccccc8c7)cc56)n4)ccc3c2)cc1]

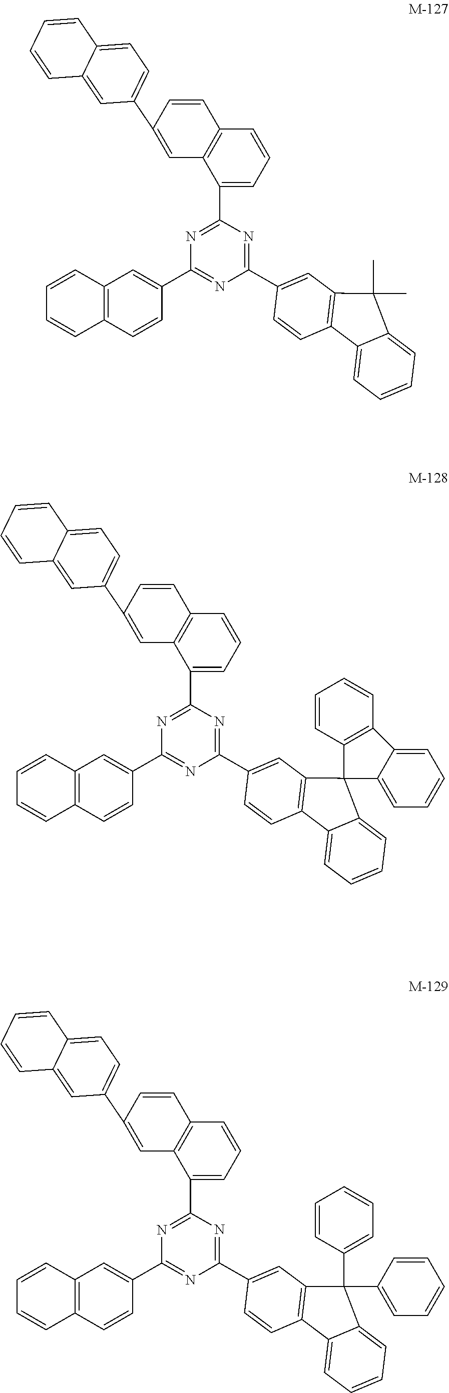 [SMILES: CC1(C)c2ccccc2-c2ccc(-c3nc(-c4ccc5ccccc5c4)nc(-c4cccc5ccc(-c6ccc7ccccc7c6)cc45)n3)cc21 ; c1ccc2c(c1)-c1ccccc1C21c2ccccc2-c2ccc(-c3nc(-c4ccc5ccccc5c4)nc(-c4cccc5ccc(-c6ccc7ccccc7c6)cc45)n3)cc21 ; c1ccc(C2(c3ccccc3)c3ccccc3-c3ccc(-c4nc(-c5ccc6ccccc6c5)nc(-c5cccc6ccc(-c7ccc8ccccc8c7)cc56)n4)cc32)cc1]

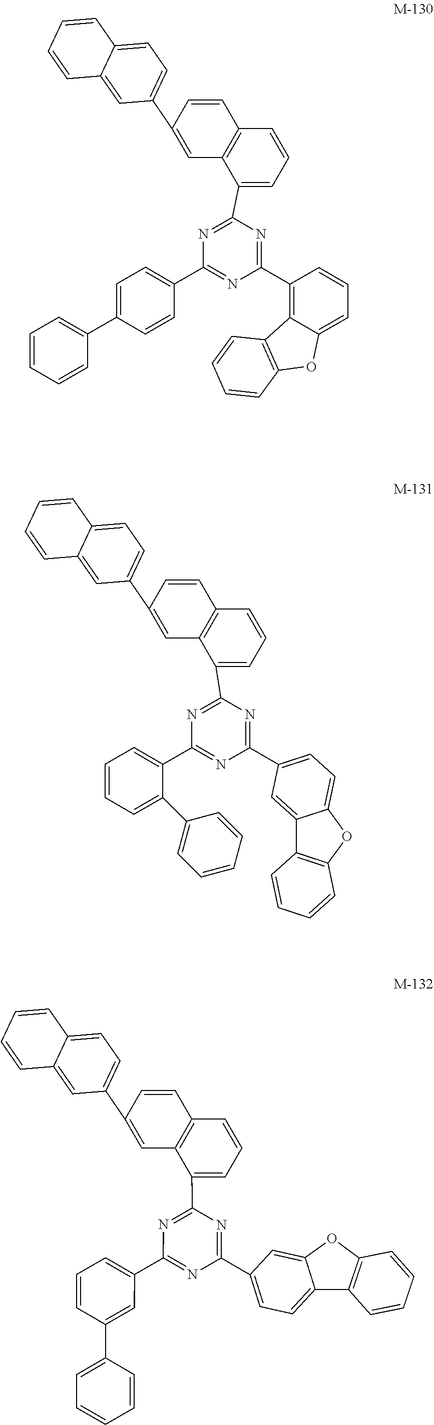 [SMILES: c1ccc(-c2ccc(-c3nc(-c4cccc5ccc(-c6ccc7ccccc7c6)cc45)nc(-c4cccc5oc6ccccc6c45)n3)cc2)cc1 ; c1ccc(-c2ccccc2-c2nc(-c3ccc4oc5ccccc5c4c3)nc(-c3cccc4ccc(-c5ccc6ccccc6c5)cc34)n2)cc1 ; c1ccc(-c2cccc(-c3nc(-c4ccc5c(c4)oc4ccccc45)nc(-c4cccc5ccc(-c6ccc7ccccc7c6)cc45)n3)c2)cc1]

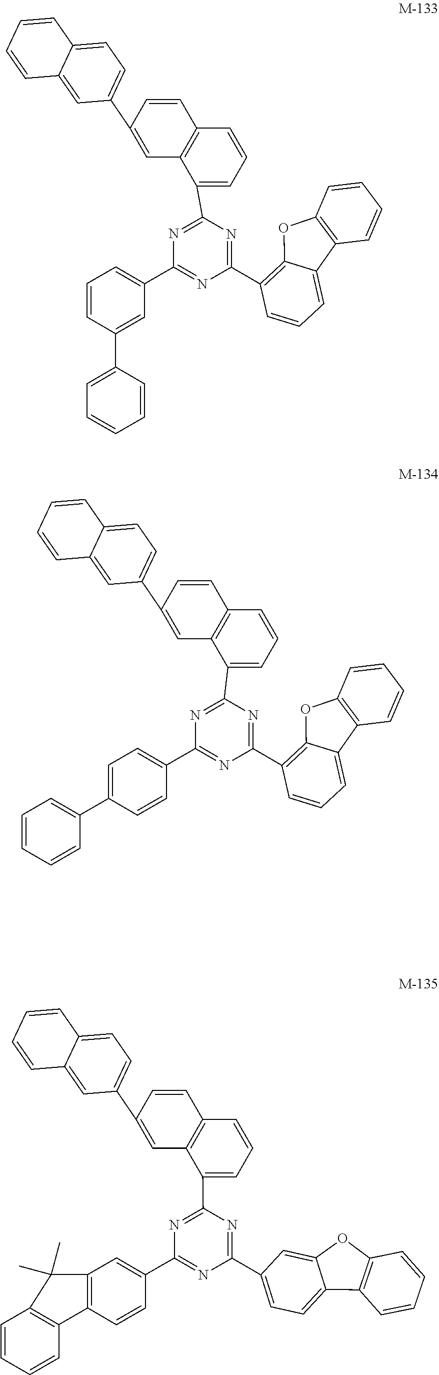 [SMILES: c1ccc(-c2cccc(-c3nc(-c4cccc5ccc(-c6ccc7ccccc7c6)cc45)nc(-c4cccc5c4oc4ccccc45)n3)c2)cc1 ; c1ccc(-c2ccc(-c3nc(-c4cccc5ccc(-c6ccc7ccccc7c6)cc45)nc(-c4cccc5c4oc4ccccc45)n3)cc2)cc1 ; CC1(C)c2ccccc2-c2ccc(-c3nc(-c4ccc5c(c4)oc4ccccc45)nc(-c4cccc5ccc(-c6ccc7ccccc7c6)cc45)n3)cc21]

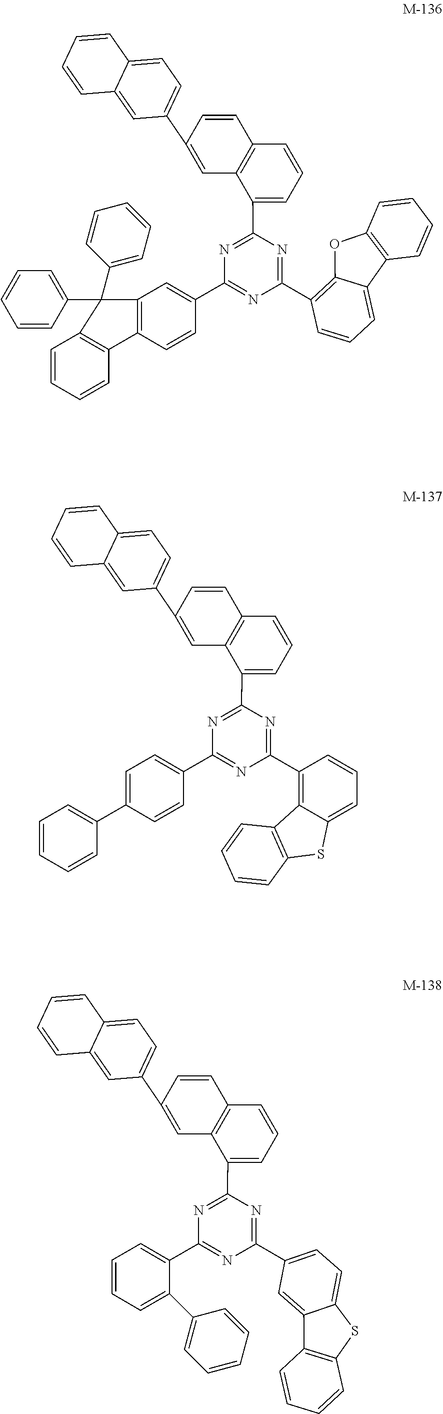 [SMILES: c1ccc(C2(c3ccccc3)c3ccccc3-c3ccc(-c4nc(-c5cccc6ccc(-c7ccc8ccccc8c7)cc56)nc(-c5cccc6c5oc5ccccc56)n4)cc32)cc1 ; c1ccc(-c2ccc(-c3nc(-c4cccc5ccc(-c6ccc7ccccc7c6)cc45)nc(-c4cccc5sc6ccccc6c45)n3)cc2)cc1 ; c1ccc(-c2ccccc2-c2nc(-c3ccc4sc5ccccc5c4c3)nc(-c3cccc4ccc(-c5ccc6ccccc6c5)cc34)n2)cc1]

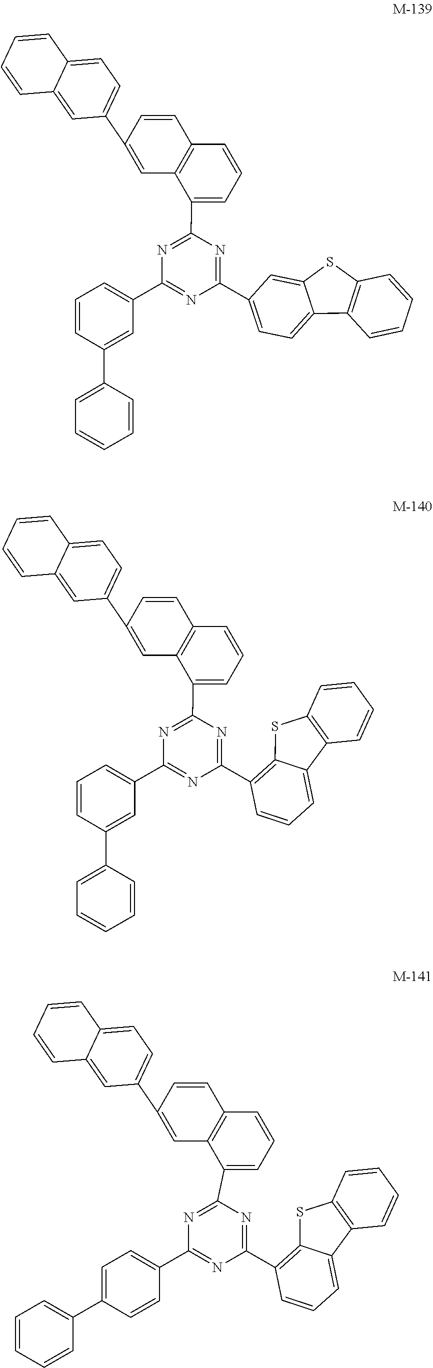 [SMILES: c1ccc(-c2cccc(-c3nc(-c4ccc5c(c4)sc4ccccc45)nc(-c4cccc5ccc(-c6ccc7ccccc7c6)cc45)n3)c2)cc1 ; c1ccc(-c2cccc(-c3nc(-c4cccc5ccc(-c6ccc7ccccc7c6)cc45)nc(-c4cccc5c4sc4ccccc45)n3)c2)cc1 ; c1ccc(-c2ccc(-c3nc(-c4cccc5ccc(-c6ccc7ccccc7c6)cc45)nc(-c4cccc5c4sc4ccccc45)n3)cc2)cc1]

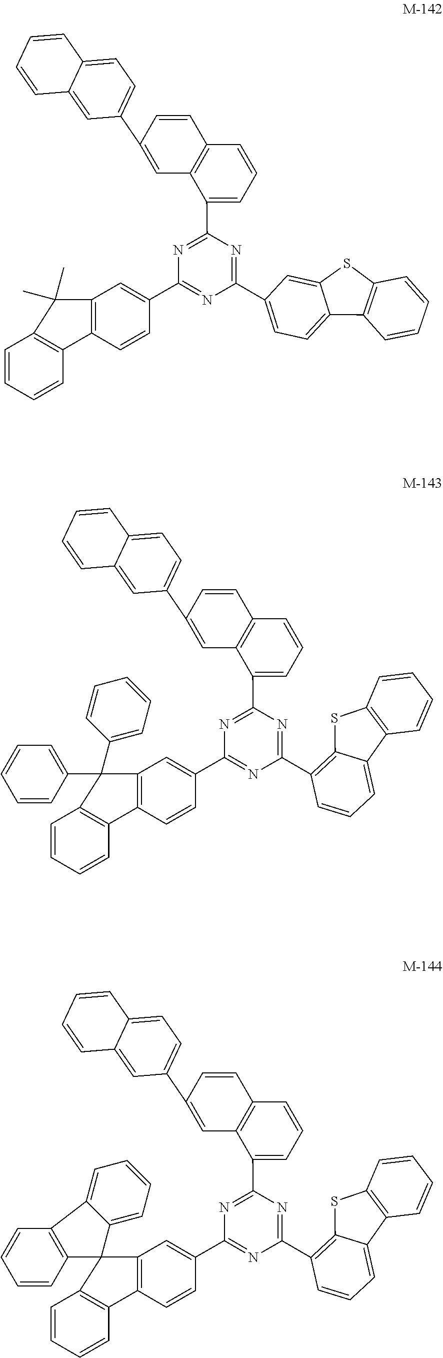 [SMILES: CC1(C)c2ccccc2-c2ccc(-c3nc(-c4ccc5c(c4)sc4ccccc45)nc(-c4cccc5ccc(-c6ccc7ccccc7c6)cc45)n3)cc21 ; c1ccc(C2(c3ccccc3)c3ccccc3-c3ccc(-c4nc(-c5cccc6ccc(-c7ccc8ccccc8c7)cc56)nc(-c5cccc6c5sc5ccccc56)n4)cc32)cc1 ; c1ccc2c(c1)-c1ccccc1C21c2ccccc2-c2ccc(-c3nc(-c4cccc5ccc(-c6ccc7ccccc7c6)cc45)nc(-c4cccc5c4sc4ccccc45)n3)cc21]

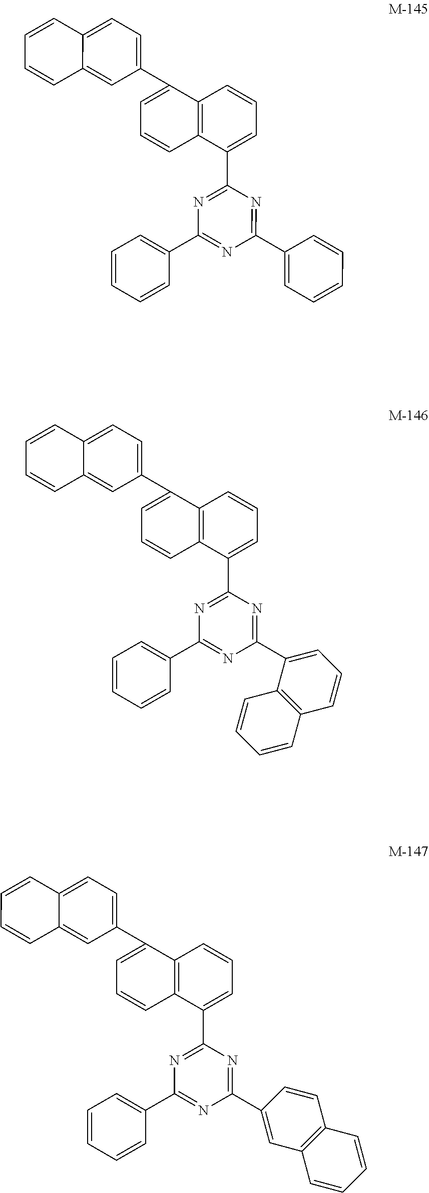 [SMILES: c1ccc(-c2nc(-c3ccccc3)nc(-c3cccc4c(-c5ccc6ccccc6c5)cccc34)n2)cc1 ; c1ccc(-c2nc(-c3cccc4ccccc34)nc(-c3cccc4c(-c5ccc6ccccc6c5)cccc34)n2)cc1 ; c1ccc(-c2nc(-c3ccc4ccccc4c3)nc(-c3cccc4c(-c5ccc6ccccc6c5)cccc34)n2)cc1]

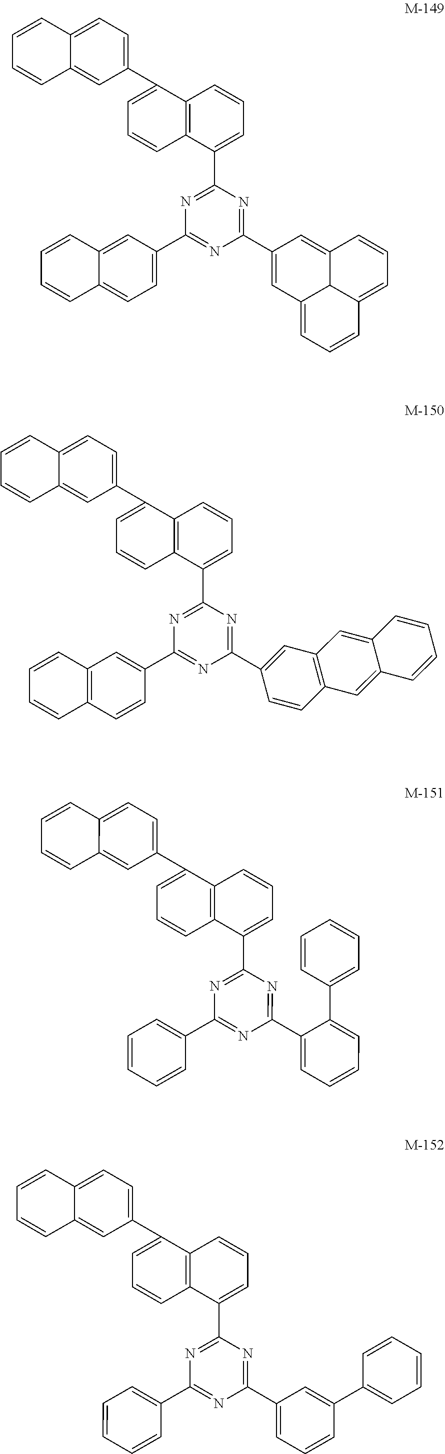 [SMILES: C1=CC2=CC=CC3=CC(c4nc(-c5ccc6ccccc6c5)nc(-c5cccc6c(-c7ccc8ccccc8c7)cccc56)n4)=CC(=C1)C23 ; c1ccc2cc(-c3nc(-c4ccc5cc6ccccc6cc5c4)nc(-c4cccc5c(-c6ccc7ccccc7c6)cccc45)n3)ccc2c1 ; c1ccc(-c2nc(-c3ccccc3-c3ccccc3)nc(-c3cccc4c(-c5ccc6ccccc6c5)cccc34)n2)cc1 ; c1ccc(-c2cccc(-c3nc(-c4ccccc4)nc(-c4cccc5c(-c6ccc7ccccc7c6)cccc45)n3)c2)cc1]

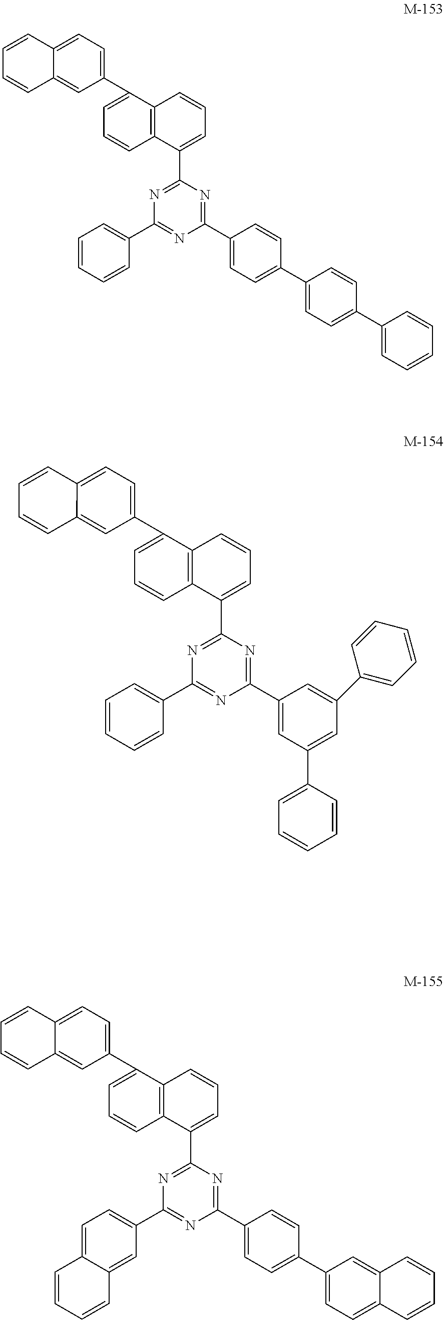 [SMILES: c1ccc(-c2ccc(-c3ccc(-c4nc(-c5ccccc5)nc(-c5cccc6c(-c7ccc8ccccc8c7)cccc56)n4)cc3)cc2)cc1 ; c1ccc(-c2cc(-c3ccccc3)cc(-c3nc(-c4ccccc4)nc(-c4cccc5c(-c6ccc7ccccc7c6)cccc45)n3)c2)cc1 ; c1ccc2cc(-c3ccc(-c4nc(-c5ccc6ccccc6c5)nc(-c5cccc6c(-c7ccc8ccccc8c7)cccc56)n4)cc3)ccc2c1]

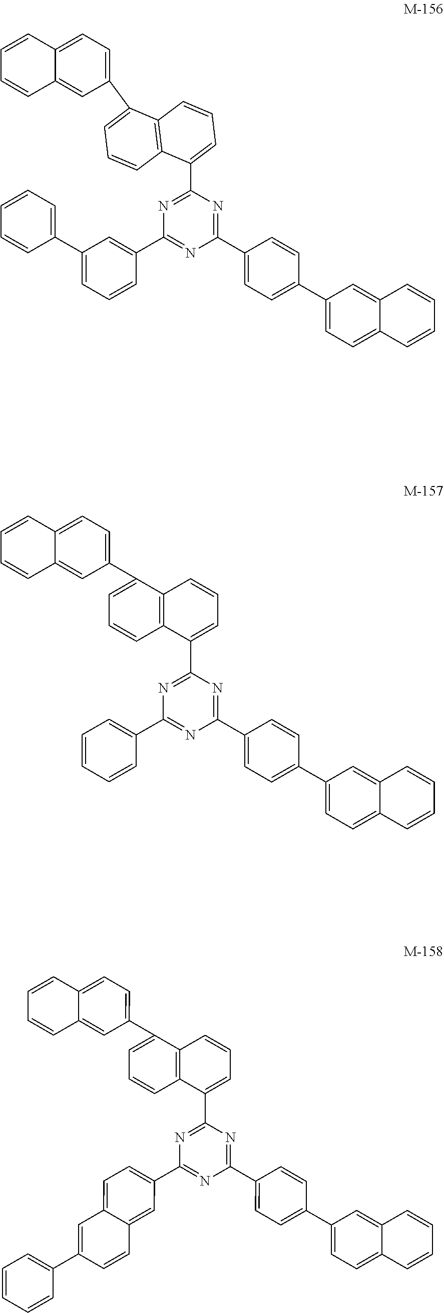 [SMILES: c1ccc(-c2cccc(-c3nc(-c4ccc(-c5ccc6ccccc6c5)cc4)nc(-c4cccc5c(-c6ccc7ccccc7c6)cccc45)n3)c2)cc1 ; c1ccc(-c2nc(-c3ccc(-c4ccc5ccccc5c4)cc3)nc(-c3cccc4c(-c5ccc6ccccc6c5)cccc34)n2)cc1 ; c1ccc(-c2ccc3cc(-c4nc(-c5ccc(-c6ccc7ccccc7c6)cc5)nc(-c5cccc6c(-c7ccc8ccccc8c7)cccc56)n4)ccc3c2)cc1]

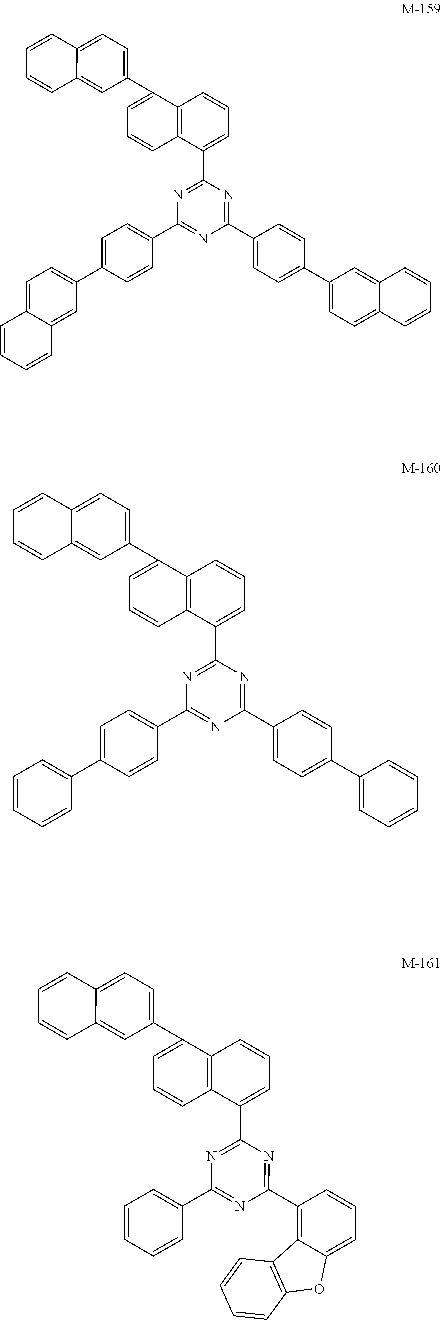 [SMILES: c1ccc2cc(-c3ccc(-c4nc(-c5ccc(-c6ccc7ccccc7c6)cc5)nc(-c5cccc6c(-c7ccc8ccccc8c7)cccc56)n4)cc3)ccc2c1 ; c1ccc(-c2ccc(-c3nc(-c4ccc(-c5ccccc5)cc4)nc(-c4cccc5c(-c6ccc7ccccc7c6)cccc45)n3)cc2)cc1 ; c1ccc(-c2nc(-c3cccc4c(-c5ccc6ccccc6c5)cccc34)nc(-c3cccc4oc5ccccc5c34)n2)cc1]

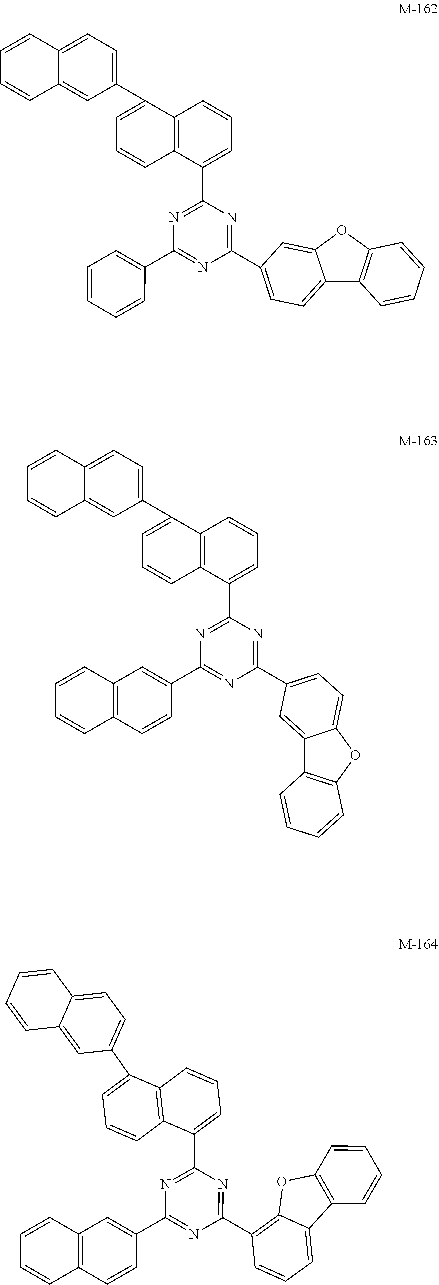 [SMILES: c1ccc(-c2nc(-c3ccc4c(c3)oc3ccccc34)nc(-c3cccc4c(-c5ccc6ccccc6c5)cccc34)n2)cc1 ; c1ccc2cc(-c3nc(-c4ccc5oc6ccccc6c5c4)nc(-c4cccc5c(-c6ccc7ccccc7c6)cccc45)n3)ccc2c1 ; c1ccc2cc(-c3nc(-c4cccc5c(-c6ccc7ccccc7c6)cccc45)nc(-c4cccc5c4oc4ccccc45)n3)ccc2c1]

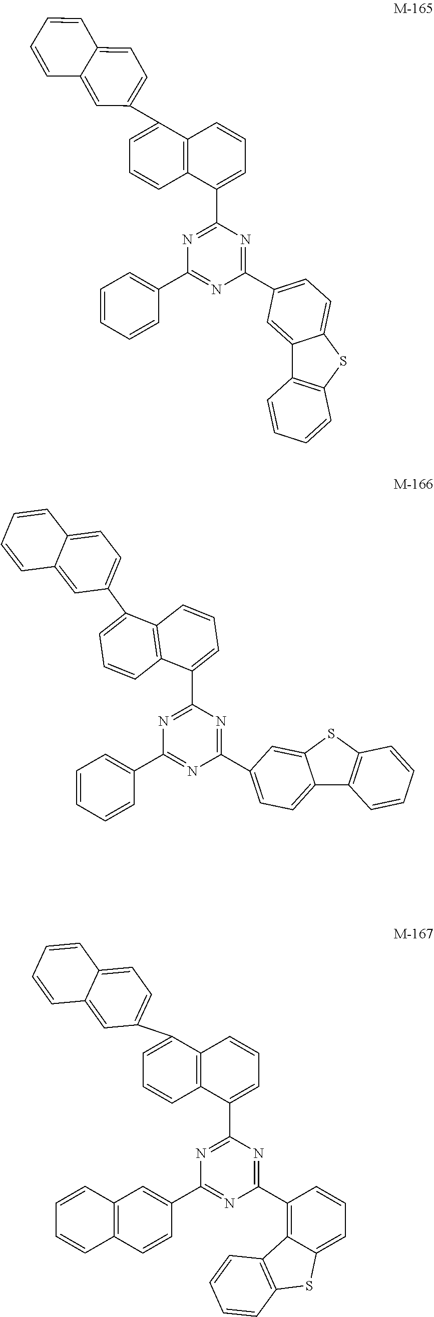 [SMILES: c1ccc(-c2nc(-c3ccc4sc5ccccc5c4c3)nc(-c3cccc4c(-c5ccc6ccccc6c5)cccc34)n2)cc1 ; c1ccc(-c2nc(-c3ccc4c(c3)sc3ccccc34)nc(-c3cccc4c(-c5ccc6ccccc6c5)cccc34)n2)cc1 ; c1ccc2cc(-c3nc(-c4cccc5c(-c6ccc7ccccc7c6)cccc45)nc(-c4cccc5sc6ccccc6c45)n3)ccc2c1]

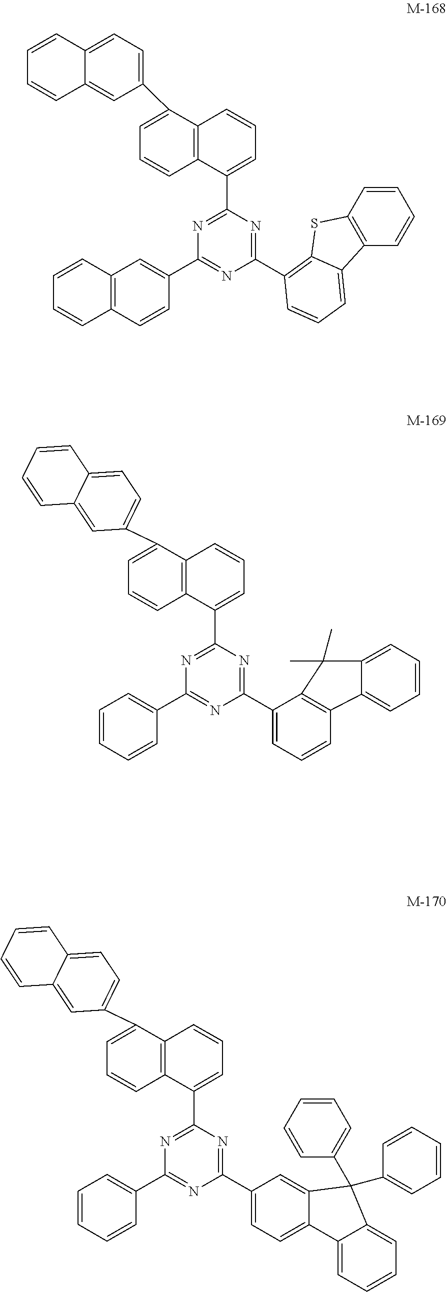 [SMILES: c1ccc2cc(-c3nc(-c4cccc5c(-c6ccc7ccccc7c6)cccc45)nc(-c4cccc5c4sc4ccccc45)n3)ccc2c1 ; CC1(C)c2ccccc2-c2cccc(-c3nc(-c4ccccc4)nc(-c4cccc5c(-c6ccc7ccccc7c6)cccc45)n3)c21 ; c1ccc(-c2nc(-c3ccc4c(c3)C(c3ccccc3)(c3ccccc3)c3ccccc3-4)nc(-c3cccc4c(-c5ccc6ccccc6c5)cccc34)n2)cc1]

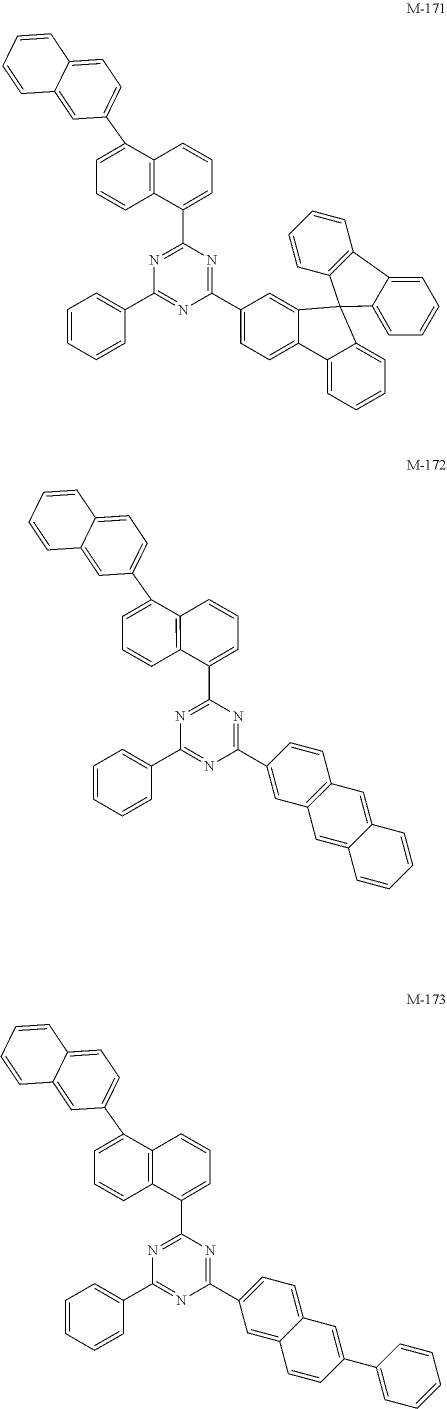 [SMILES: c1ccc(-c2nc(-c3ccc4c(c3)C3(c5ccccc5-c5ccccc53)c3ccccc3-4)nc(-c3cccc4c(-c5ccc6ccccc6c5)cccc34)n2)cc1 ; c1ccc(-c2nc(-c3ccc4cc5ccccc5cc4c3)nc(-c3cccc4c(-c5ccc6ccccc6c5)cccc34)n2)cc1 ; c1ccc(-c2ccc3cc(-c4nc(-c5ccccc5)nc(-c5cccc6c(-c7ccc8ccccc8c7)cccc56)n4)ccc3c2)cc1]

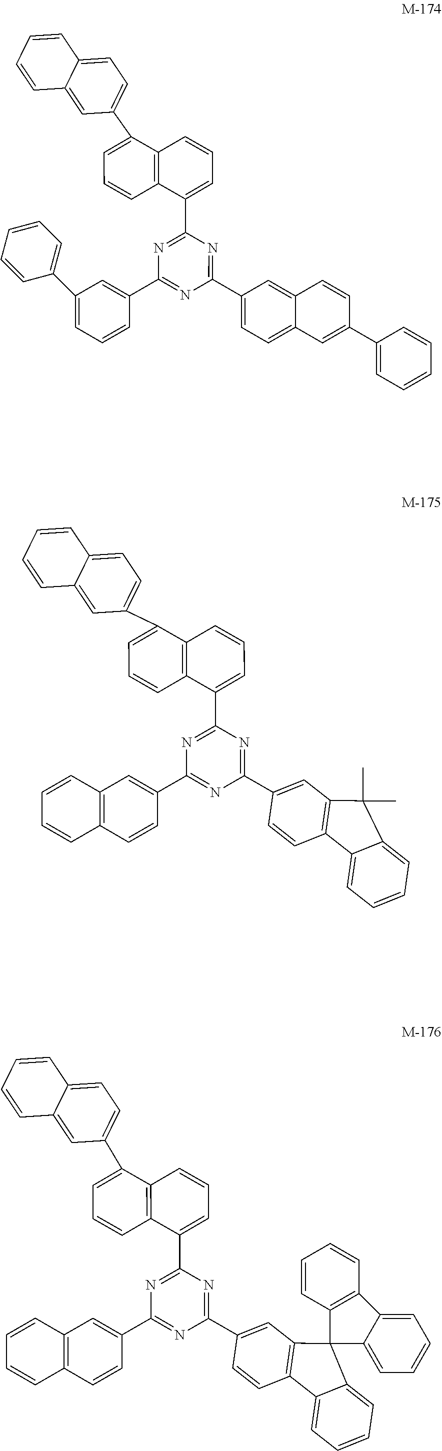 [SMILES: c1ccc(-c2cccc(-c3nc(-c4ccc5cc(-c6ccccc6)ccc5c4)nc(-c4cccc5c(-c6ccc7ccccc7c6)cccc45)n3)c2)cc1 ; CC1(C)c2ccccc2-c2ccc(-c3nc(-c4ccc5ccccc5c4)nc(-c4cccc5c(-c6ccc7ccccc7c6)cccc45)n3)cc21 ; c1ccc2c(c1)-c1ccccc1C21c2ccccc2-c2ccc(-c3nc(-c4ccc5ccccc5c4)nc(-c4cccc5c(-c6ccc7ccccc7c6)cccc45)n3)cc21]

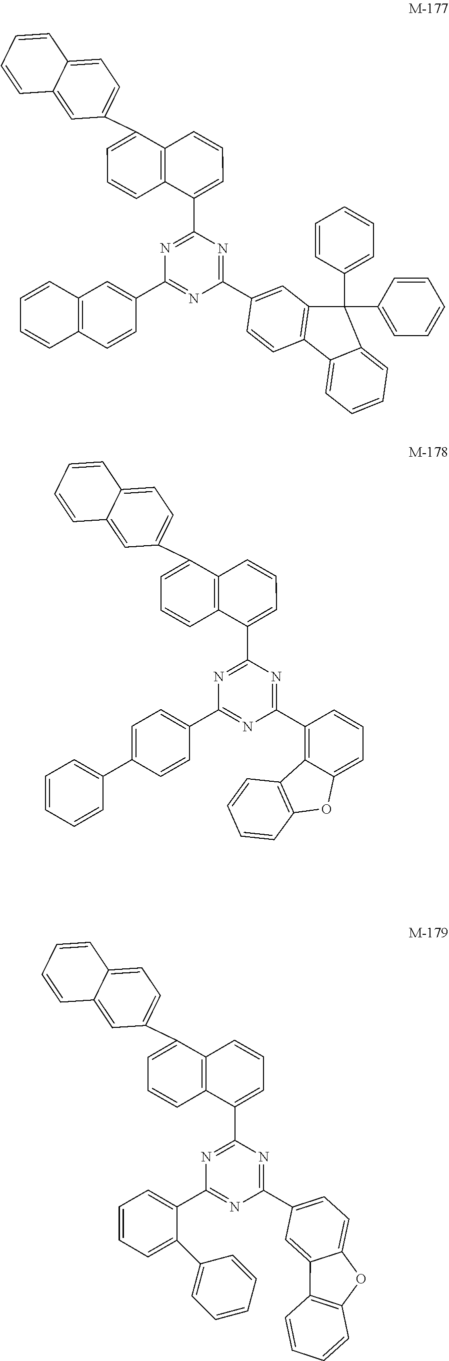 [SMILES: c1ccc(C2(c3ccccc3)c3ccccc3-c3ccc(-c4nc(-c5ccc6ccccc6c5)nc(-c5cccc6c(-c7ccc8ccccc8c7)cccc56)n4)cc32)cc1 ; c1ccc(-c2ccc(-c3nc(-c4cccc5c(-c6ccc7ccccc7c6)cccc45)nc(-c4cccc5oc6ccccc6c45)n3)cc2)cc1 ; c1ccc(-c2ccccc2-c2nc(-c3ccc4oc5ccccc5c4c3)nc(-c3cccc4c(-c5ccc6ccccc6c5)cccc34)n2)cc1]

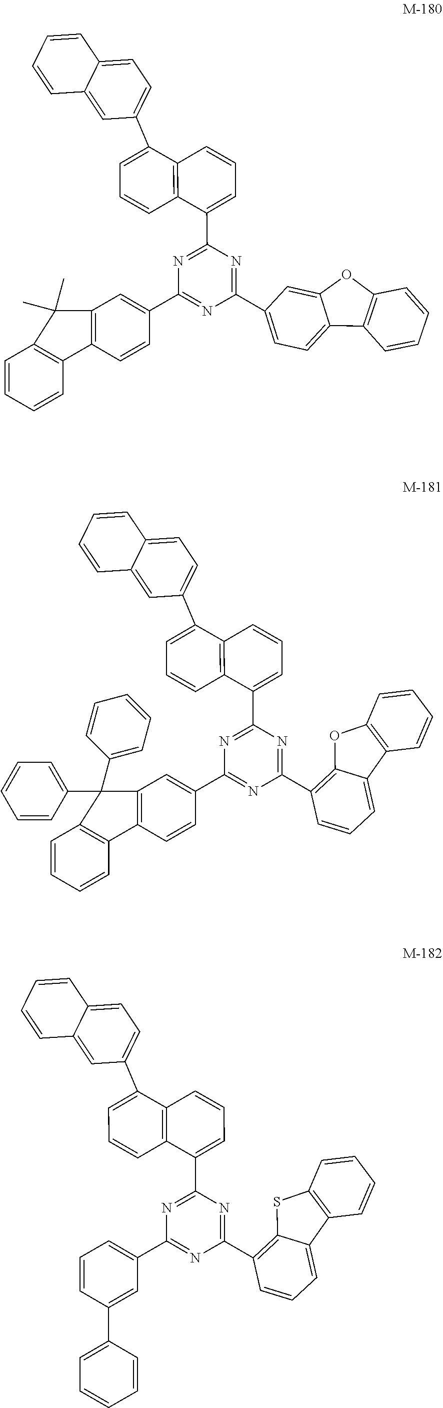 [SMILES: CC1(C)c2ccccc2-c2ccc(-c3nc(-c4ccc5c(c4)oc4ccccc45)nc(-c4cccc5c(-c6ccc7ccccc7c6)cccc45)n3)cc21 ; c1ccc(C2(c3ccccc3)c3ccccc3-c3ccc(-c4nc(-c5cccc6c(-c7ccc8ccccc8c7)cccc56)nc(-c5cccc6c5oc5ccccc56)n4)cc32)cc1 ; c1ccc(-c2cccc(-c3nc(-c4cccc5c(-c6ccc7ccccc7c6)cccc45)nc(-c4cccc5c4sc4ccccc45)n3)c2)cc1]

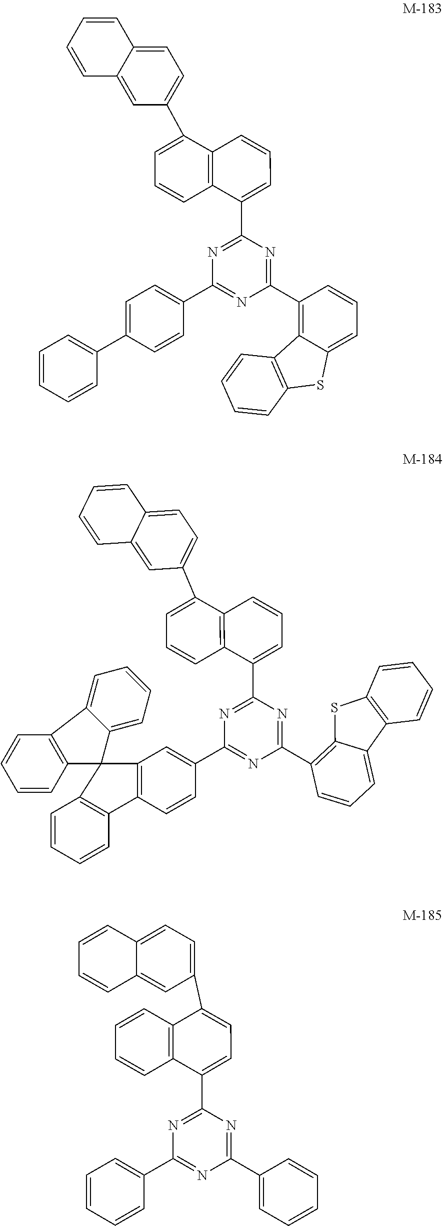 [SMILES: c1ccc(-c2ccc(-c3nc(-c4cccc5c(-c6ccc7ccccc7c6)cccc45)nc(-c4cccc5sc6ccccc6c45)n3)cc2)cc1 ; c1ccc2c(c1)-c1ccccc1C21c2ccccc2-c2ccc(-c3nc(-c4cccc5c(-c6ccc7ccccc7c6)cccc45)nc(-c4cccc5c4sc4ccccc45)n3)cc21 ; c1ccc(-c2nc(-c3ccccc3)nc(-c3ccc(-c4ccc5ccccc5c4)c4ccccc34)n2)cc1]

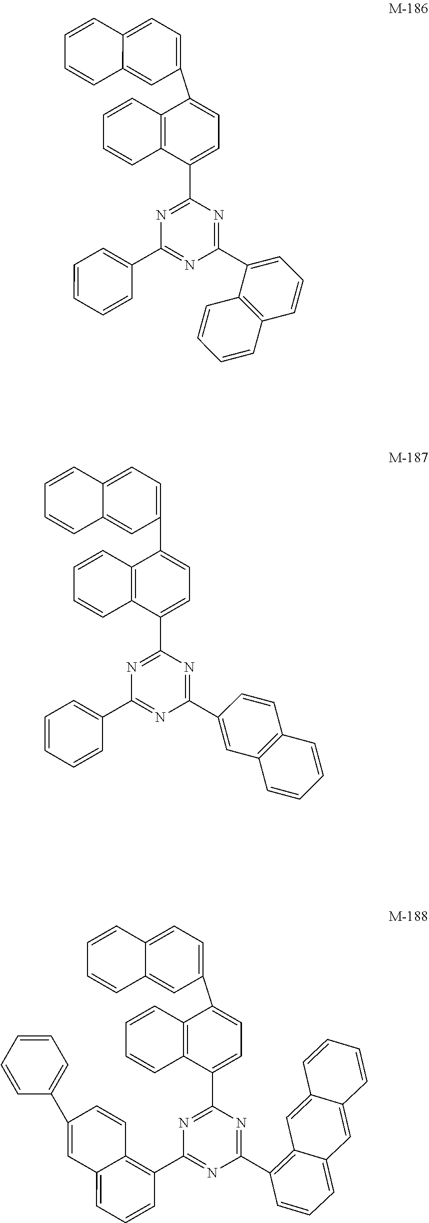 [SMILES: c1ccc(-c2nc(-c3cccc4ccccc34)nc(-c3ccc(-c4ccc5ccccc5c4)c4ccccc34)n2)cc1 ; c1ccc(-c2nc(-c3ccc4ccccc4c3)nc(-c3ccc(-c4ccc5ccccc5c4)c4ccccc34)n2)cc1 ; c1ccc(-c2ccc3c(-c4nc(-c5cccc6cc7ccccc7cc56)nc(-c5ccc(-c6ccc7ccccc7c6)c6ccccc56)n4)cccc3c2)cc1]

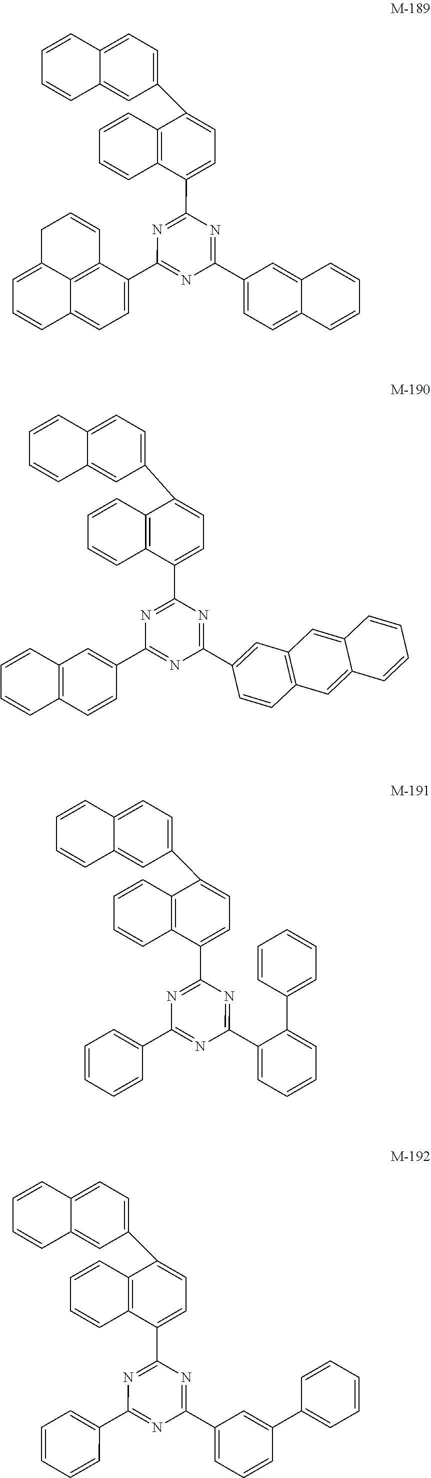 [SMILES: C1=Cc2c(-c3nc(-c4ccc5ccccc5c4)nc(-c4ccc(-c5ccc6ccccc6c5)c5ccccc45)n3)ccc3cccc(c23)C1 ; c1ccc2cc(-c3nc(-c4ccc5cc6ccccc6cc5c4)nc(-c4ccc(-c5ccc6ccccc6c5)c5ccccc45)n3)ccc2c1 ; c1ccc(-c2nc(-c3ccccc3-c3ccccc3)nc(-c3ccc(-c4ccc5ccccc5c4)c4ccccc34)n2)cc1 ; c1ccc(-c2cccc(-c3nc(-c4ccccc4)nc(-c4ccc(-c5ccc6ccccc6c5)c5ccccc45)n3)c2)cc1]

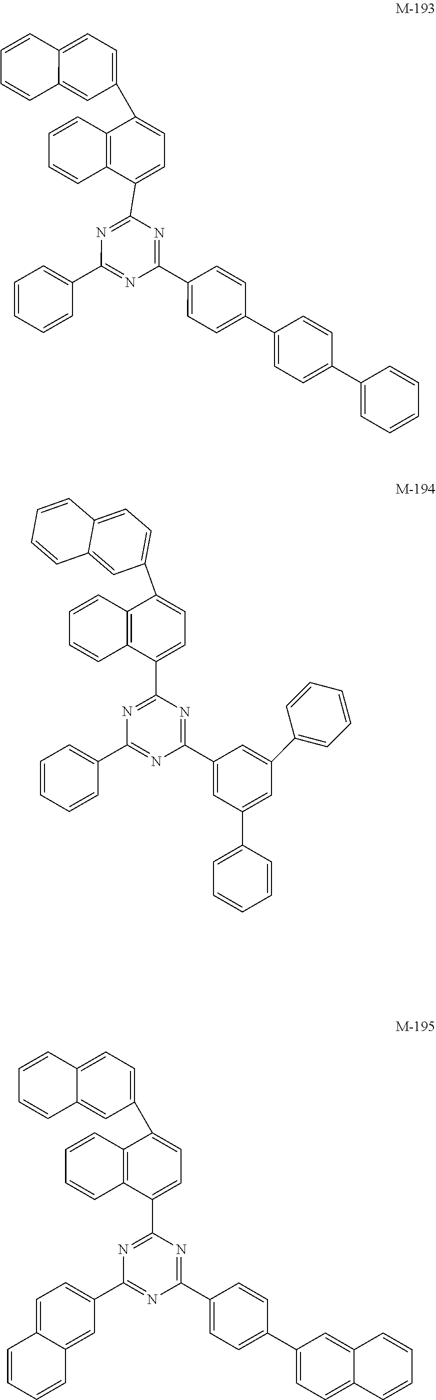 [SMILES: c1ccc(-c2ccc(-c3ccc(-c4nc(-c5ccccc5)nc(-c5ccc(-c6ccc7ccccc7c6)c6ccccc56)n4)cc3)cc2)cc1 ; c1ccc(-c2cc(-c3ccccc3)cc(-c3nc(-c4ccccc4)nc(-c4ccc(-c5ccc6ccccc6c5)c5ccccc45)n3)c2)cc1 ; c1ccc2cc(-c3ccc(-c4nc(-c5ccc6ccccc6c5)nc(-c5ccc(-c6ccc7ccccc7c6)c6ccccc56)n4)cc3)ccc2c1]

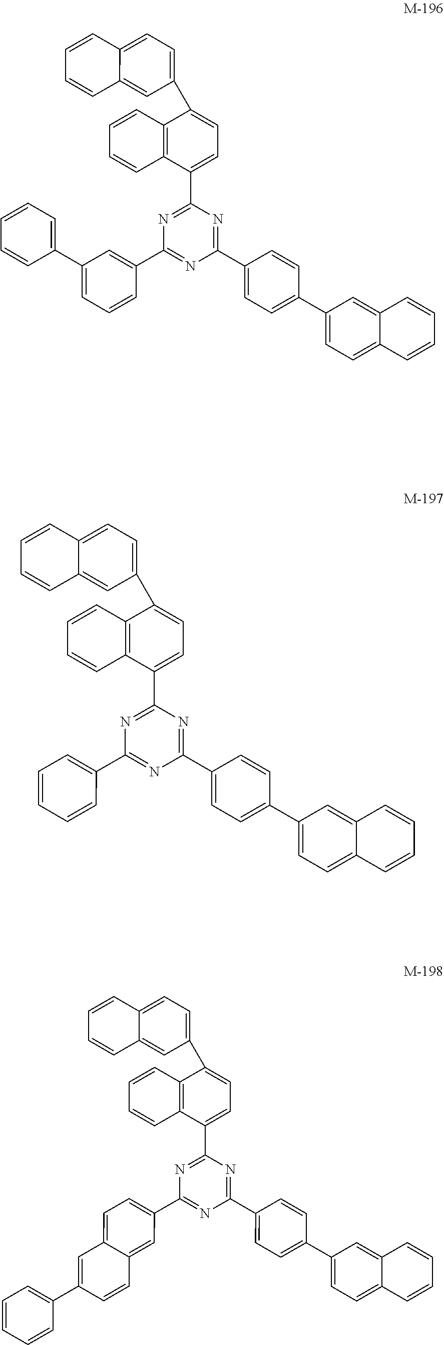 [SMILES: c1ccc(-c2cccc(-c3nc(-c4ccc(-c5ccc6ccccc6c5)cc4)nc(-c4ccc(-c5ccc6ccccc6c5)c5ccccc45)n3)c2)cc1 ; c1ccc(-c2nc(-c3ccc(-c4ccc5ccccc5c4)cc3)nc(-c3ccc(-c4ccc5ccccc5c4)c4ccccc34)n2)cc1 ; c1ccc(-c2ccc3cc(-c4nc(-c5ccc(-c6ccc7ccccc7c6)cc5)nc(-c5ccc(-c6ccc7ccccc7c6)c6ccccc56)n4)ccc3c2)cc1]

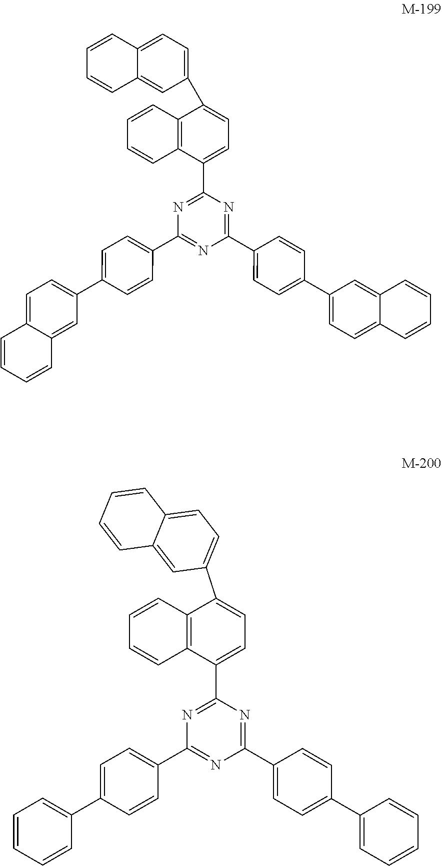 [SMILES: c1ccc2cc(-c3ccc(-c4nc(-c5ccc(-c6ccc7ccccc7c6)cc5)nc(-c5ccc(-c6ccc7ccccc7c6)c6ccccc56)n4)cc3)ccc2c1 ; c1ccc(-c2ccc(-c3nc(-c4ccc(-c5ccccc5)cc4)nc(-c4ccc(-c5ccc6ccccc6c5)c5ccccc45)n3)cc2)cc1]

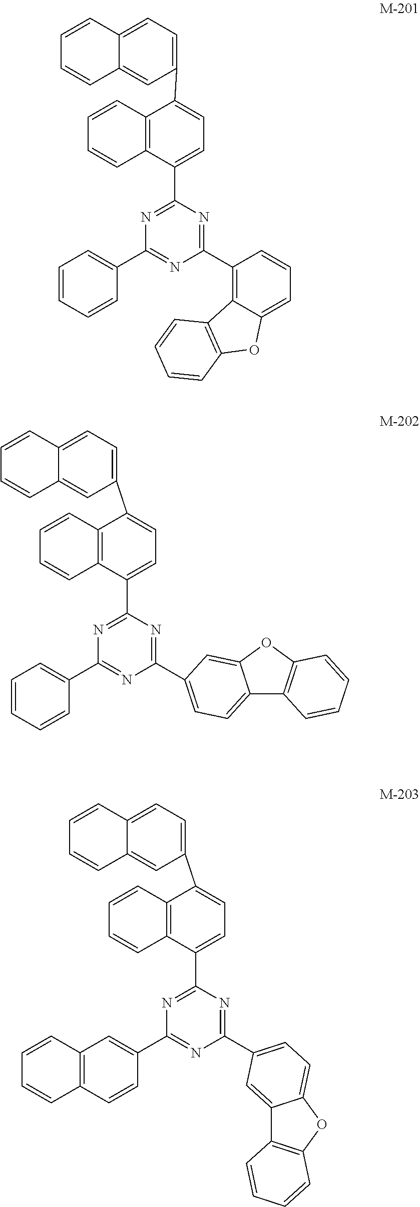 [SMILES: c1ccc(-c2nc(-c3ccc(-c4ccc5ccccc5c4)c4ccccc34)nc(-c3cccc4oc5ccccc5c34)n2)cc1 ; c1ccc(-c2nc(-c3ccc4c(c3)oc3ccccc34)nc(-c3ccc(-c4ccc5ccccc5c4)c4ccccc34)n2)cc1 ; c1ccc2cc(-c3nc(-c4ccc5oc6ccccc6c5c4)nc(-c4ccc(-c5ccc6ccccc6c5)c5ccccc45)n3)ccc2c1]

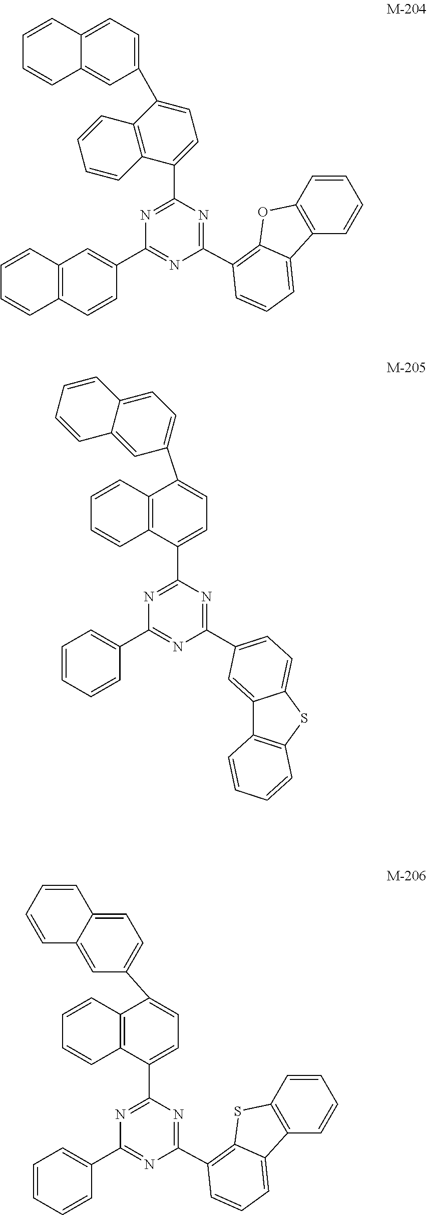 [SMILES: c1ccc2cc(-c3nc(-c4ccc(-c5ccc6ccccc6c5)c5ccccc45)nc(-c4cccc5c4oc4ccccc45)n3)ccc2c1 ; c1ccc(-c2nc(-c3ccc4sc5ccccc5c4c3)nc(-c3ccc(-c4ccc5ccccc5c4)c4ccccc34)n2)cc1 ; c1ccc(-c2nc(-c3ccc(-c4ccc5ccccc5c4)c4ccccc34)nc(-c3cccc4c3sc3ccccc34)n2)cc1]

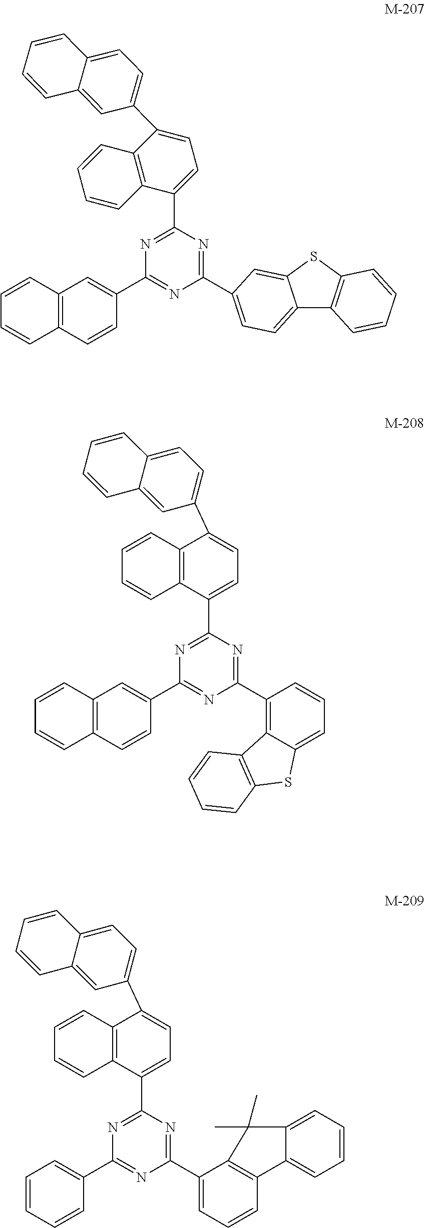 [SMILES: c1ccc2cc(-c3nc(-c4ccc5c(c4)sc4ccccc45)nc(-c4ccc(-c5ccc6ccccc6c5)c5ccccc45)n3)ccc2c1 ; c1ccc2cc(-c3nc(-c4ccc(-c5ccc6ccccc6c5)c5ccccc45)nc(-c4cccc5sc6ccccc6c45)n3)ccc2c1 ; CC1(C)c2ccccc2-c2cccc(-c3nc(-c4ccccc4)nc(-c4ccc(-c5ccc6ccccc6c5)c5ccccc45)n3)c21]

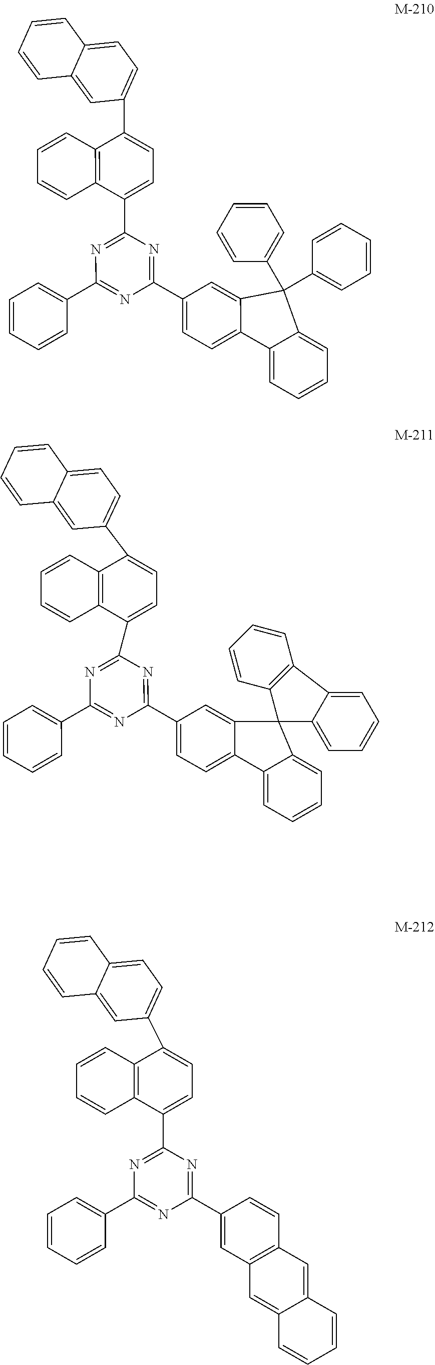 [SMILES: c1ccc(-c2nc(-c3ccc4c(c3)C(c3ccccc3)(c3ccccc3)c3ccccc3-4)nc(-c3ccc(-c4ccc5ccccc5c4)c4ccccc34)n2)cc1 ; c1ccc(-c2nc(-c3ccc4c(c3)C3(c5ccccc5-c5ccccc53)c3ccccc3-4)nc(-c3ccc(-c4ccc5ccccc5c4)c4ccccc34)n2)cc1 ; c1ccc(-c2nc(-c3ccc4cc5ccccc5cc4c3)nc(-c3ccc(-c4ccc5ccccc5c4)c4ccccc34)n2)cc1]

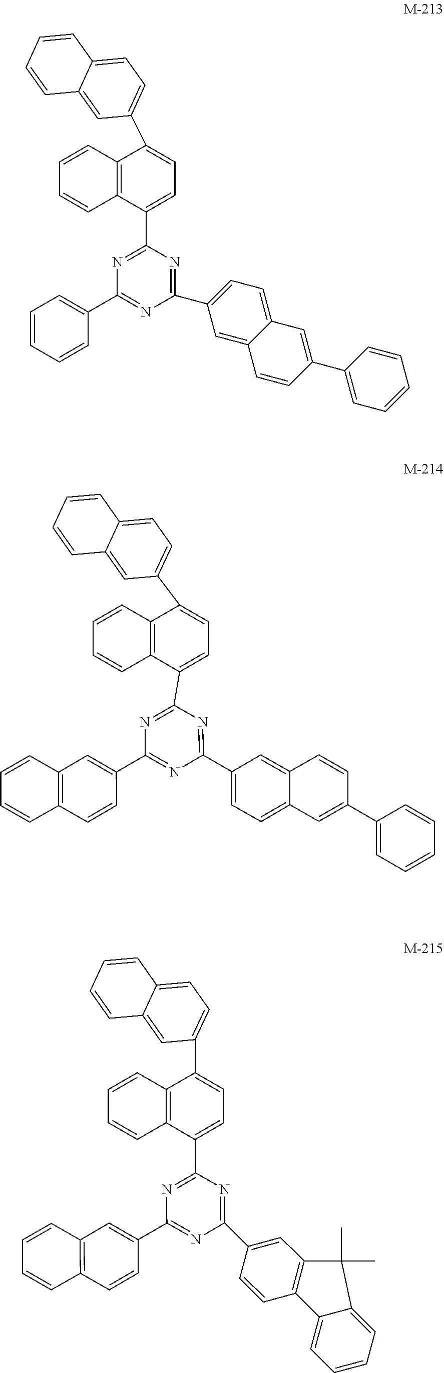 [SMILES: c1ccc(-c2ccc3cc(-c4nc(-c5ccccc5)nc(-c5ccc(-c6ccc7ccccc7c6)c6ccccc56)n4)ccc3c2)cc1 ; c1ccc(-c2ccc3cc(-c4nc(-c5ccc6ccccc6c5)nc(-c5ccc(-c6ccc7ccccc7c6)c6ccccc56)n4)ccc3c2)cc1 ; CC1(C)c2ccccc2-c2ccc(-c3nc(-c4ccc5ccccc5c4)nc(-c4ccc(-c5ccc6ccccc6c5)c5ccccc45)n3)cc21]

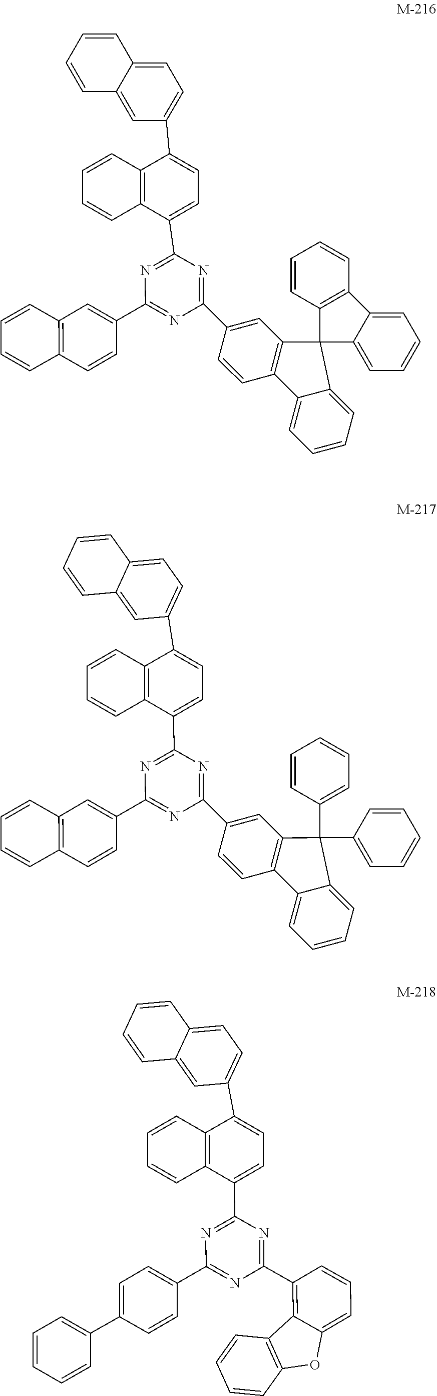 [SMILES: c1ccc2c(c1)-c1ccccc1C21c2ccccc2-c2ccc(-c3nc(-c4ccc5ccccc5c4)nc(-c4ccc(-c5ccc6ccccc6c5)c5ccccc45)n3)cc21 ; c1ccc(C2(c3ccccc3)c3ccccc3-c3ccc(-c4nc(-c5ccc6ccccc6c5)nc(-c5ccc(-c6ccc7ccccc7c6)c6ccccc56)n4)cc32)cc1 ; c1ccc(-c2ccc(-c3nc(-c4ccc(-c5ccc6ccccc6c5)c5ccccc45)nc(-c4cccc5oc6ccccc6c45)n3)cc2)cc1]

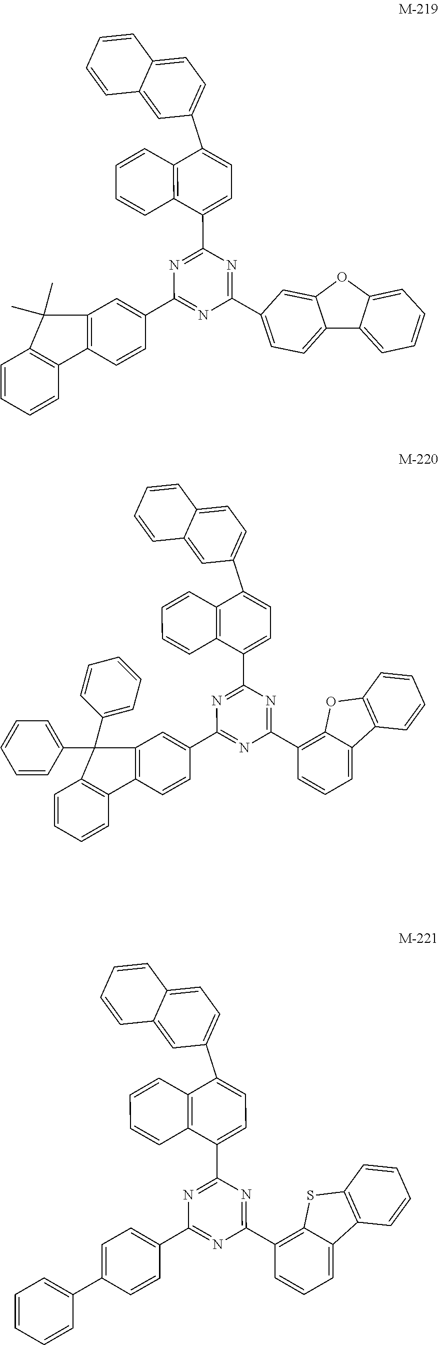 [SMILES: CC1(C)c2ccccc2-c2ccc(-c3nc(-c4ccc5c(c4)oc4ccccc45)nc(-c4ccc(-c5ccc6ccccc6c5)c5ccccc45)n3)cc21 ; c1ccc(C2(c3ccccc3)c3ccccc3-c3ccc(-c4nc(-c5ccc(-c6ccc7ccccc7c6)c6ccccc56)nc(-c5cccc6c5oc5ccccc56)n4)cc32)cc1 ; c1ccc(-c2ccc(-c3nc(-c4ccc(-c5ccc6ccccc6c5)c5ccccc45)nc(-c4cccc5c4sc4ccccc45)n3)cc2)cc1]

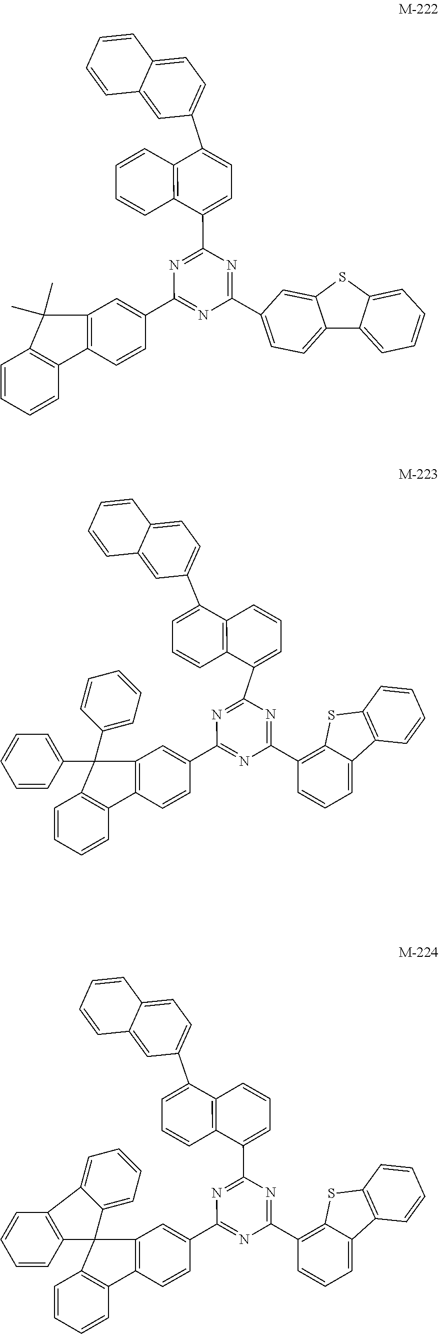 [SMILES: CC1(C)c2ccccc2-c2ccc(-c3nc(-c4ccc5c(c4)sc4ccccc45)nc(-c4ccc(-c5ccc6ccccc6c5)c5ccccc45)n3)cc21 ; c1ccc(C2(c3ccccc3)c3ccccc3-c3ccc(-c4nc(-c5cccc6c(-c7ccc8ccccc8c7)cccc56)nc(-c5cccc6c5sc5ccccc56)n4)cc32)cc1 ; c1ccc2c(c1)-c1ccccc1C21c2ccccc2-c2ccc(-c3nc(-c4cccc5c(-c6ccc7ccccc7c6)cccc45)nc(-c4cccc5c4sc4ccccc45)n3)cc21]

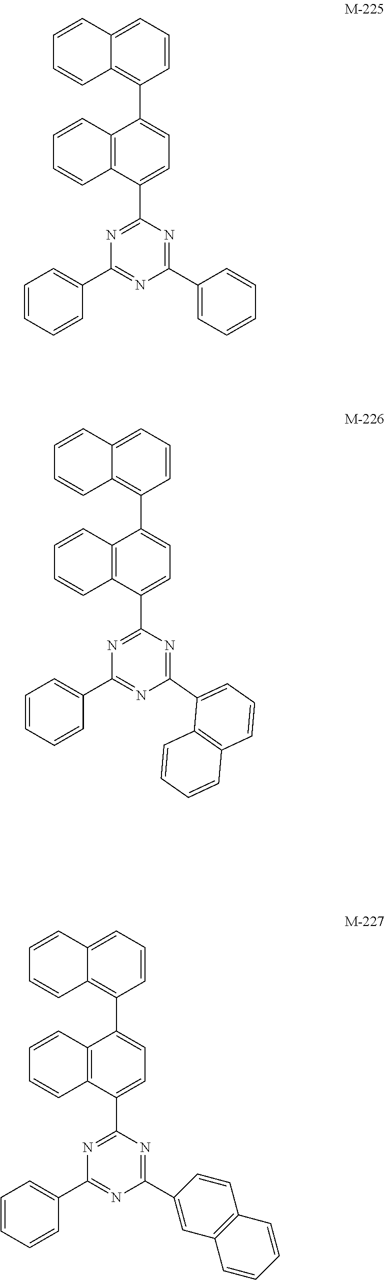 [SMILES: c1ccc(-c2nc(-c3ccccc3)nc(-c3ccc(-c4cccc5ccccc45)c4ccccc34)n2)cc1 ; c1ccc(-c2nc(-c3cccc4ccccc34)nc(-c3ccc(-c4cccc5ccccc45)c4ccccc34)n2)cc1 ; c1ccc(-c2nc(-c3ccc4ccccc4c3)nc(-c3ccc(-c4cccc5ccccc45)c4ccccc34)n2)cc1]

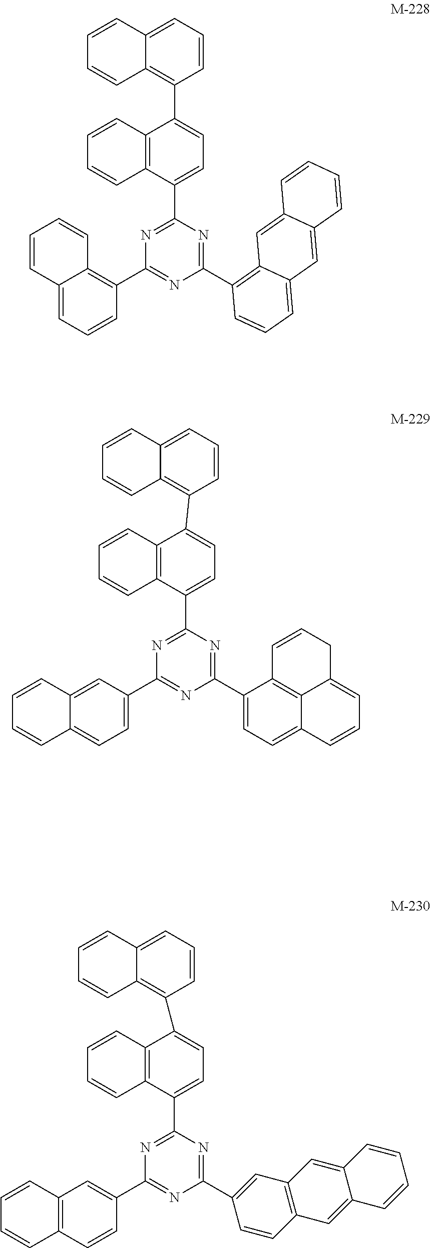 [SMILES: c1ccc2cc3c(-c4nc(-c5cccc6ccccc56)nc(-c5ccc(-c6cccc7ccccc67)c6ccccc56)n4)cccc3cc2c1 ; C1=Cc2c(-c3nc(-c4ccc5ccccc5c4)nc(-c4ccc(-c5cccc6ccccc56)c5ccccc45)n3)ccc3cccc(c23)C1 ; c1ccc2cc(-c3nc(-c4ccc5cc6ccccc6cc5c4)nc(-c4ccc(-c5cccc6ccccc56)c5ccccc45)n3)ccc2c1]

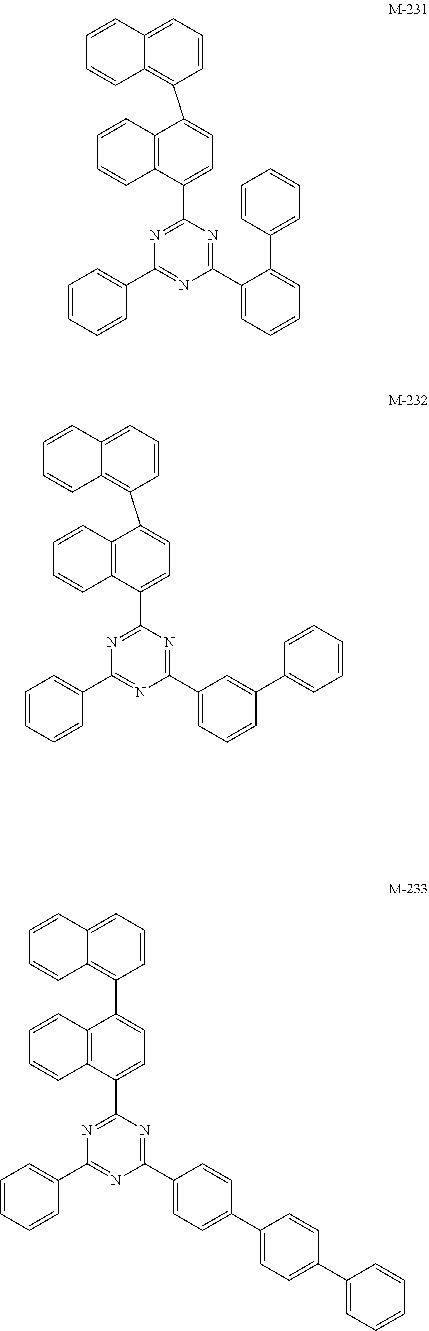 [SMILES: c1ccc(-c2nc(-c3ccccc3-c3ccccc3)nc(-c3ccc(-c4cccc5ccccc45)c4ccccc34)n2)cc1 ; c1ccc(-c2cccc(-c3nc(-c4ccccc4)nc(-c4ccc(-c5cccc6ccccc56)c5ccccc45)n3)c2)cc1 ; c1ccc(-c2ccc(-c3ccc(-c4nc(-c5ccccc5)nc(-c5ccc(-c6cccc7ccccc67)c6ccccc56)n4)cc3)cc2)cc1]

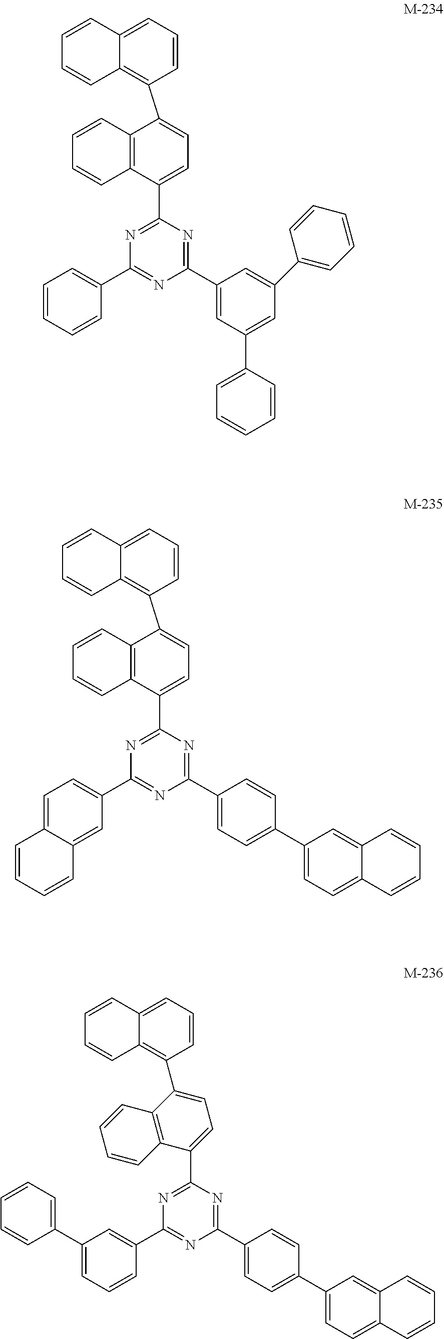 [SMILES: c1ccc(-c2cc(-c3ccccc3)cc(-c3nc(-c4ccccc4)nc(-c4ccc(-c5cccc6ccccc56)c5ccccc45)n3)c2)cc1 ; c1ccc2cc(-c3ccc(-c4nc(-c5ccc6ccccc6c5)nc(-c5ccc(-c6cccc7ccccc67)c6ccccc56)n4)cc3)ccc2c1 ; c1ccc(-c2cccc(-c3nc(-c4ccc(-c5ccc6ccccc6c5)cc4)nc(-c4ccc(-c5cccc6ccccc56)c5ccccc45)n3)c2)cc1]

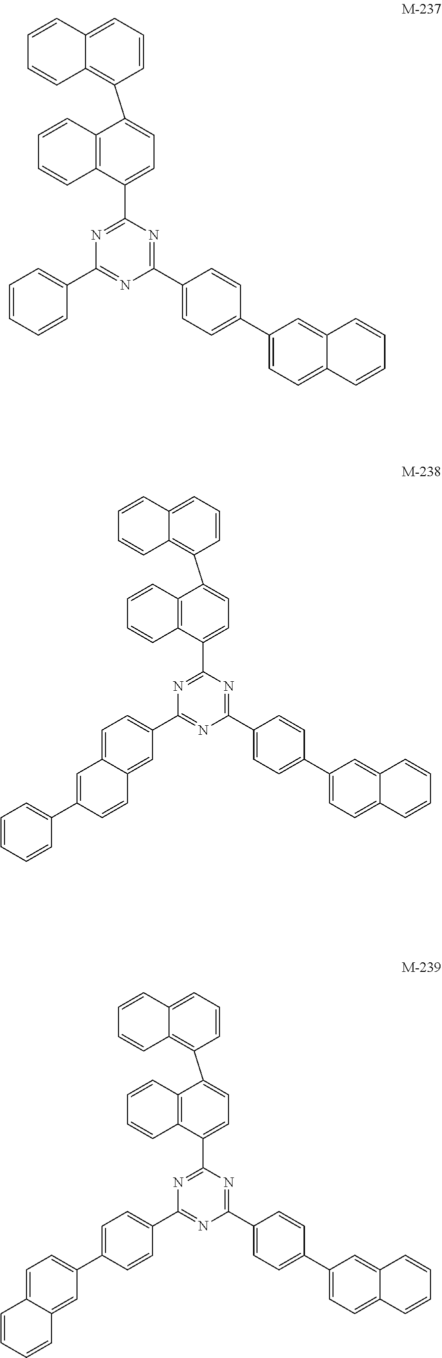 [SMILES: c1ccc(-c2nc(-c3ccc(-c4ccc5ccccc5c4)cc3)nc(-c3ccc(-c4cccc5ccccc45)c4ccccc34)n2)cc1 ; c1ccc(-c2ccc3cc(-c4nc(-c5ccc(-c6ccc7ccccc7c6)cc5)nc(-c5ccc(-c6cccc7ccccc67)c6ccccc56)n4)ccc3c2)cc1 ; c1ccc2cc(-c3ccc(-c4nc(-c5ccc(-c6ccc7ccccc7c6)cc5)nc(-c5ccc(-c6cccc7ccccc67)c6ccccc56)n4)cc3)ccc2c1]

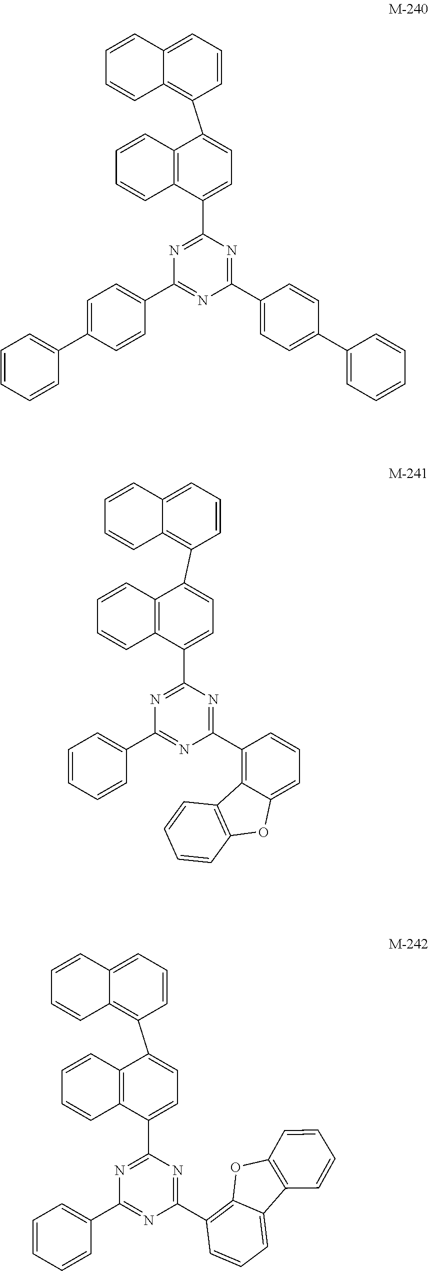 [SMILES: c1ccc(-c2ccc(-c3nc(-c4ccc(-c5ccccc5)cc4)nc(-c4ccc(-c5cccc6ccccc56)c5ccccc45)n3)cc2)cc1 ; c1ccc(-c2nc(-c3ccc(-c4cccc5ccccc45)c4ccccc34)nc(-c3cccc4oc5ccccc5c34)n2)cc1 ; c1ccc(-c2nc(-c3ccc(-c4cccc5ccccc45)c4ccccc34)nc(-c3cccc4c3oc3ccccc34)n2)cc1]

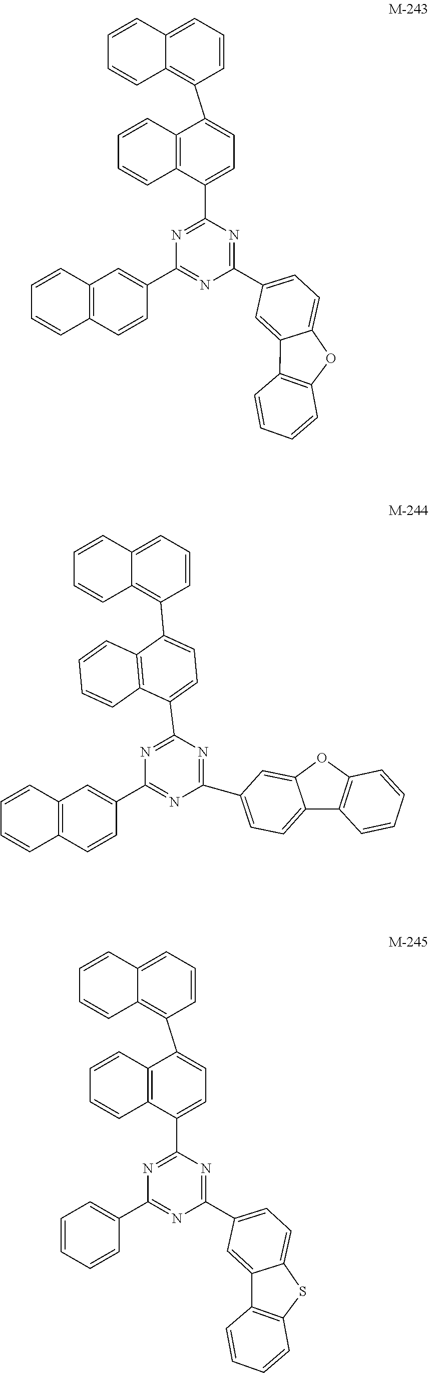 [SMILES: c1ccc2cc(-c3nc(-c4ccc5oc6ccccc6c5c4)nc(-c4ccc(-c5cccc6ccccc56)c5ccccc45)n3)ccc2c1 ; c1ccc2cc(-c3nc(-c4ccc5c(c4)oc4ccccc45)nc(-c4ccc(-c5cccc6ccccc56)c5ccccc45)n3)ccc2c1 ; c1ccc(-c2nc(-c3ccc4sc5ccccc5c4c3)nc(-c3ccc(-c4cccc5ccccc45)c4ccccc34)n2)cc1]

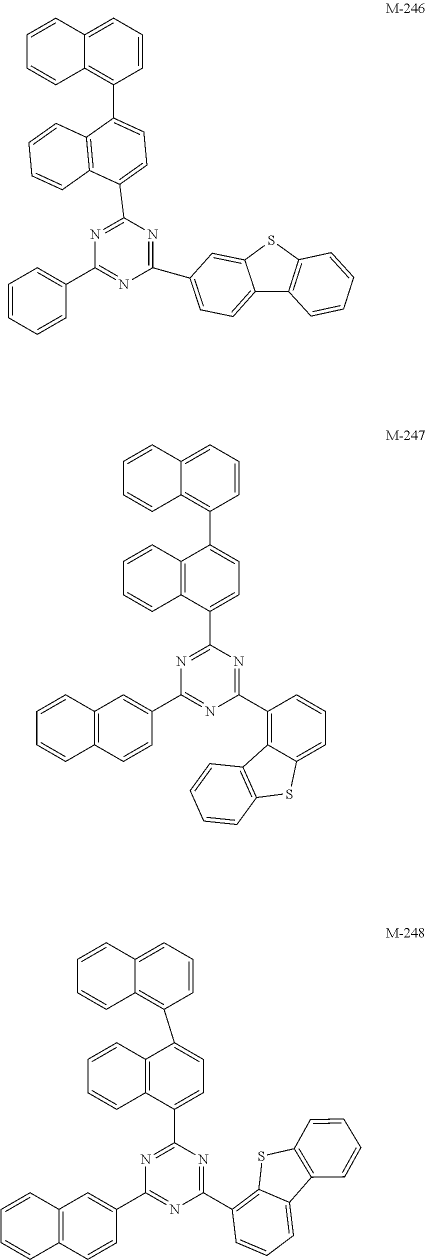 [SMILES: c1ccc(-c2nc(-c3ccc4c(c3)sc3ccccc34)nc(-c3ccc(-c4cccc5ccccc45)c4ccccc34)n2)cc1 ; c1ccc2cc(-c3nc(-c4ccc(-c5cccc6ccccc56)c5ccccc45)nc(-c4cccc5sc6ccccc6c45)n3)ccc2c1 ; c1ccc2cc(-c3nc(-c4ccc(-c5cccc6ccccc56)c5ccccc45)nc(-c4cccc5c4sc4ccccc45)n3)ccc2c1]

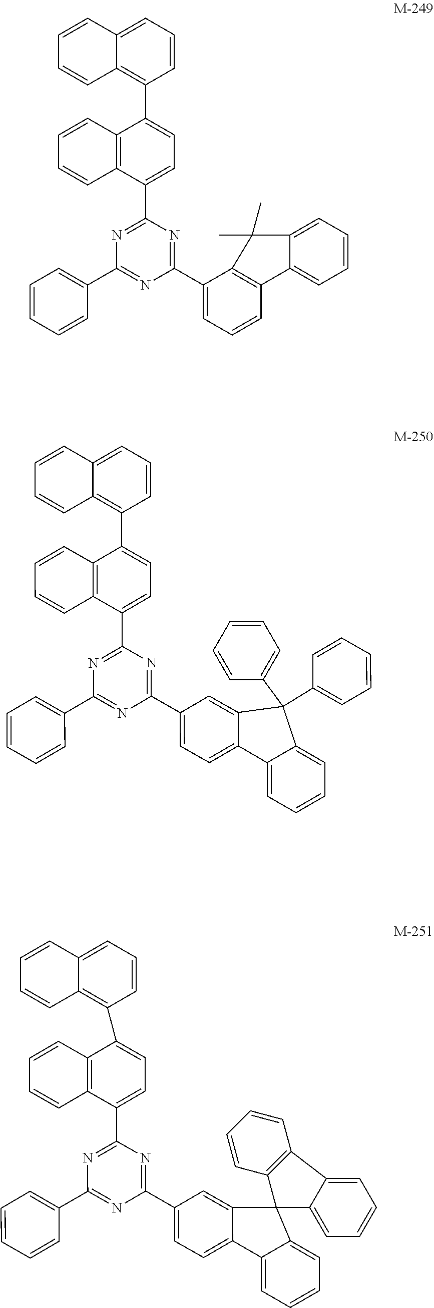 [SMILES: CC1(C)c2ccccc2-c2cccc(-c3nc(-c4ccccc4)nc(-c4ccc(-c5cccc6ccccc56)c5ccccc45)n3)c21 ; c1ccc(-c2nc(-c3ccc4c(c3)C(c3ccccc3)(c3ccccc3)c3ccccc3-4)nc(-c3ccc(-c4cccc5ccccc45)c4ccccc34)n2)cc1 ; c1ccc(-c2nc(-c3ccc4c(c3)C3(c5ccccc5-c5ccccc53)c3ccccc3-4)nc(-c3ccc(-c4cccc5ccccc45)c4ccccc34)n2)cc1]

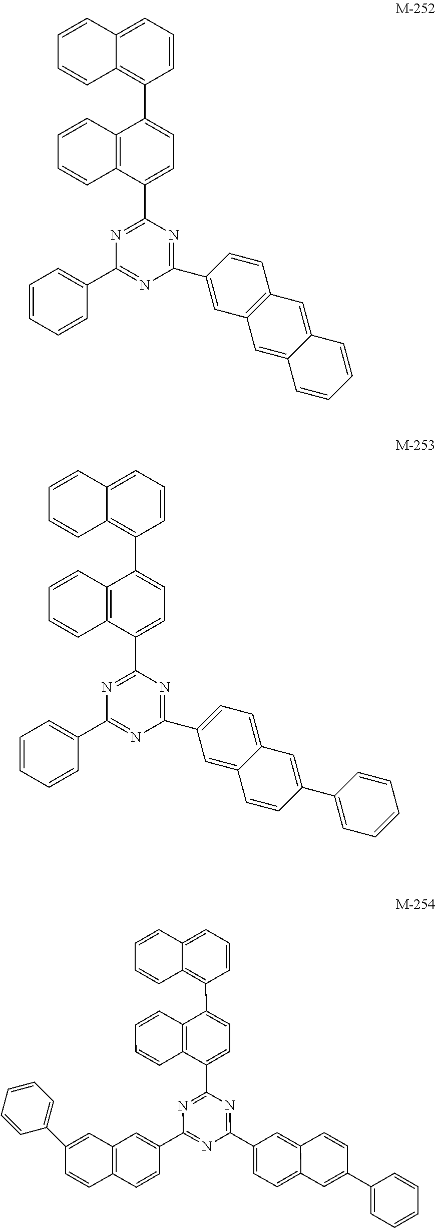 [SMILES: c1ccc(-c2nc(-c3ccc4cc5ccccc5cc4c3)nc(-c3ccc(-c4cccc5ccccc45)c4ccccc34)n2)cc1 ; c1ccc(-c2ccc3cc(-c4nc(-c5ccccc5)nc(-c5ccc(-c6cccc7ccccc67)c6ccccc56)n4)ccc3c2)cc1 ; c1ccc(-c2ccc3cc(-c4nc(-c5ccc6ccc(-c7ccccc7)cc6c5)nc(-c5ccc(-c6cccc7ccccc67)c6ccccc56)n4)ccc3c2)cc1]

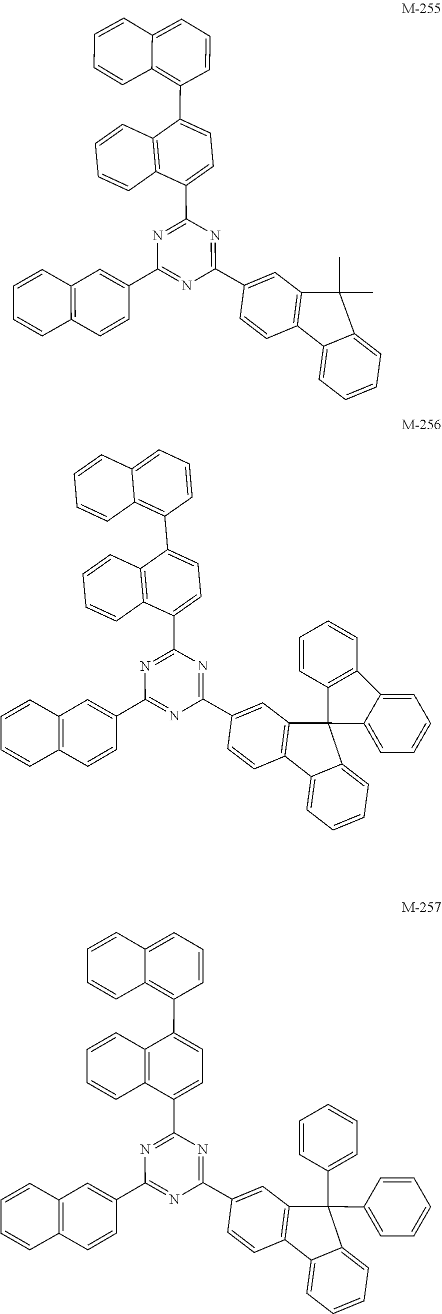 [SMILES: CC1(C)c2ccccc2-c2ccc(-c3nc(-c4ccc5ccccc5c4)nc(-c4ccc(-c5cccc6ccccc56)c5ccccc45)n3)cc21 ; c1ccc2c(c1)-c1ccccc1C21c2ccccc2-c2ccc(-c3nc(-c4ccc5ccccc5c4)nc(-c4ccc(-c5cccc6ccccc56)c5ccccc45)n3)cc21 ; c1ccc(C2(c3ccccc3)c3ccccc3-c3ccc(-c4nc(-c5ccc6ccccc6c5)nc(-c5ccc(-c6cccc7ccccc67)c6ccccc56)n4)cc32)cc1]

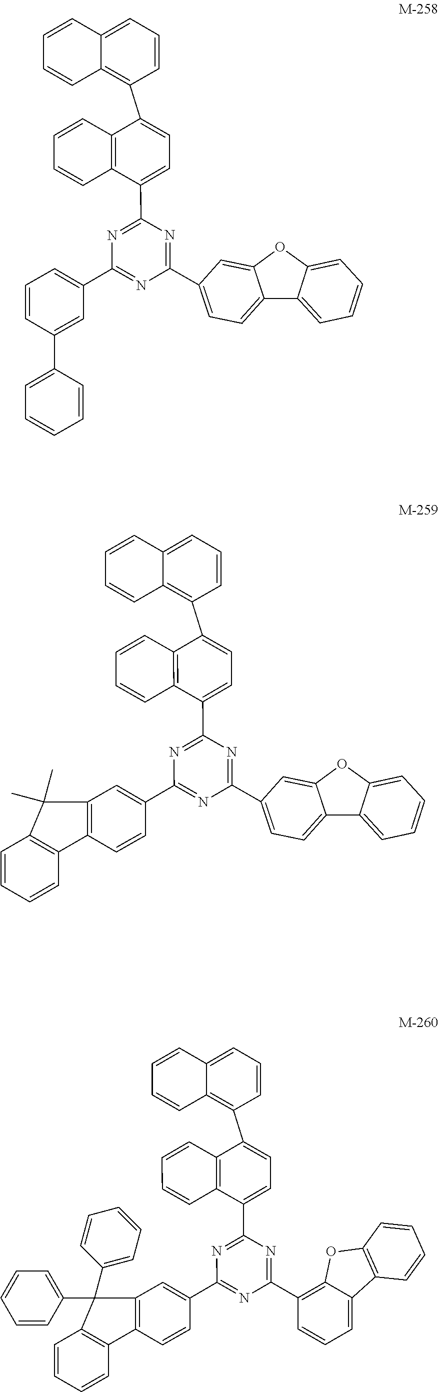 [SMILES: c1ccc(-c2cccc(-c3nc(-c4ccc5c(c4)oc4ccccc45)nc(-c4ccc(-c5cccc6ccccc56)c5ccccc45)n3)c2)cc1 ; CC1(C)c2ccccc2-c2ccc(-c3nc(-c4ccc5c(c4)oc4ccccc45)nc(-c4ccc(-c5cccc6ccccc56)c5ccccc45)n3)cc21 ; c1ccc(C2(c3ccccc3)c3ccccc3-c3ccc(-c4nc(-c5ccc(-c6cccc7ccccc67)c6ccccc56)nc(-c5cccc6c5oc5ccccc56)n4)cc32)cc1]

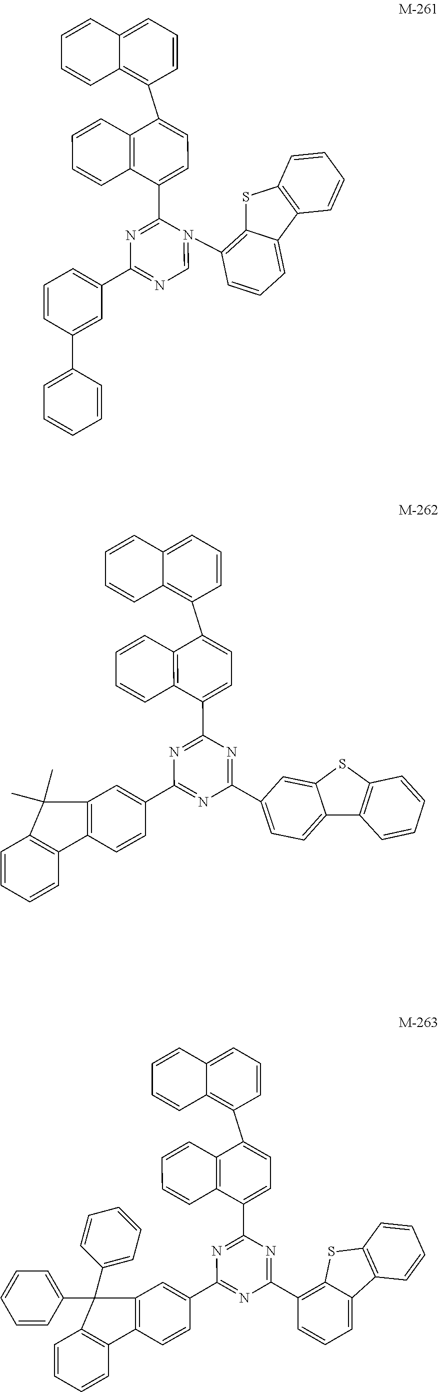

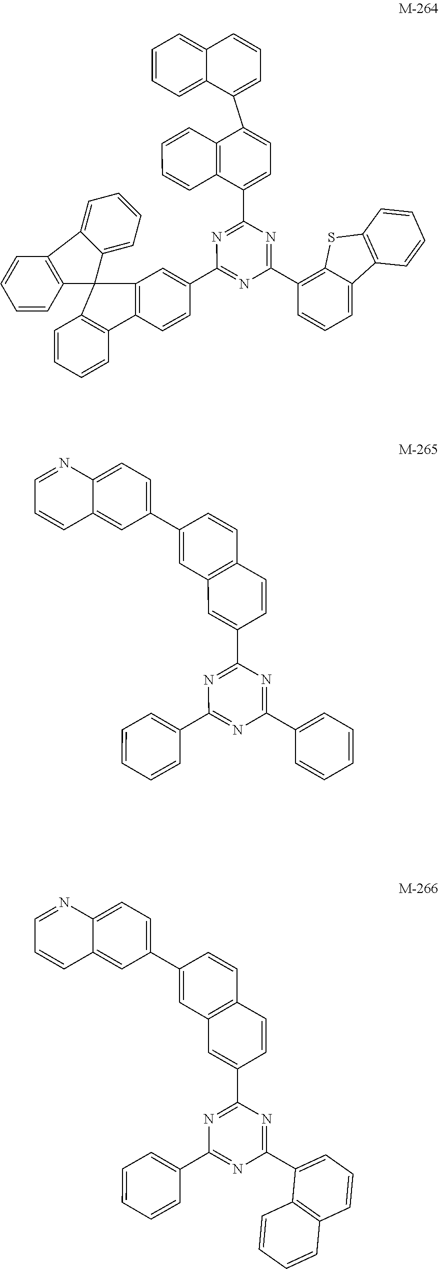 [SMILES: c1ccc2c(c1)-c1ccccc1C21c2ccccc2-c2ccc(-c3nc(-c4ccc(-c5cccc6ccccc56)c5ccccc45)nc(-c4cccc5c4sc4ccccc45)n3)cc21 ; c1ccc(-c2nc(-c3ccccc3)nc(-c3ccc4ccc(-c5ccc6ncccc6c5)cc4c3)n2)cc1 ; c1ccc(-c2nc(-c3ccc4ccc(-c5ccc6ncccc6c5)cc4c3)nc(-c3cccc4ccccc34)n2)cc1]

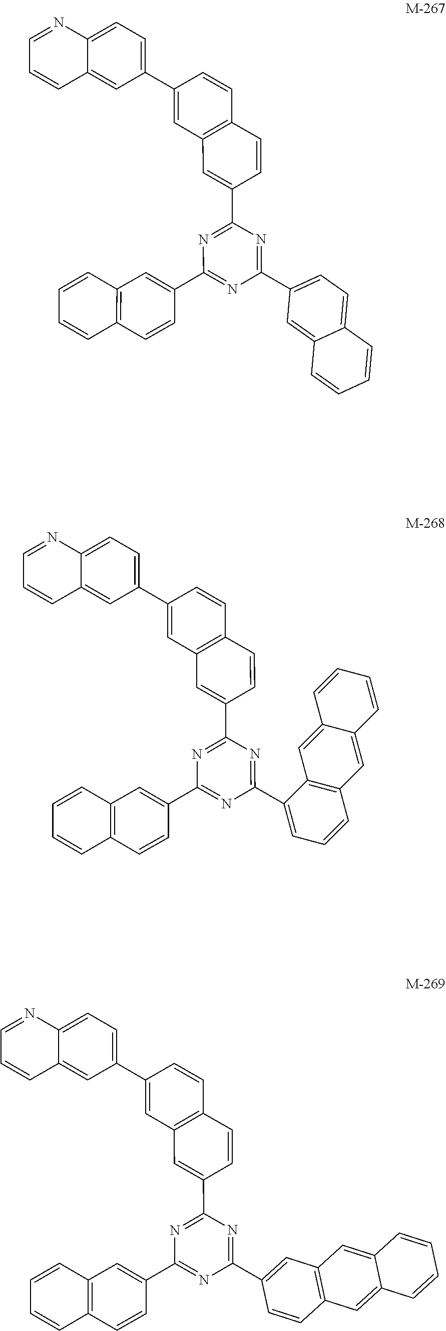 [SMILES: c1ccc2cc(-c3nc(-c4ccc5ccccc5c4)nc(-c4ccc5ccc(-c6ccc7ncccc7c6)cc5c4)n3)ccc2c1 ; c1ccc2cc(-c3nc(-c4ccc5ccc(-c6ccc7ncccc7c6)cc5c4)nc(-c4cccc5cc6ccccc6cc45)n3)ccc2c1 ; c1ccc2cc(-c3nc(-c4ccc5ccc(-c6ccc7ncccc7c6)cc5c4)nc(-c4ccc5cc6ccccc6cc5c4)n3)ccc2c1]

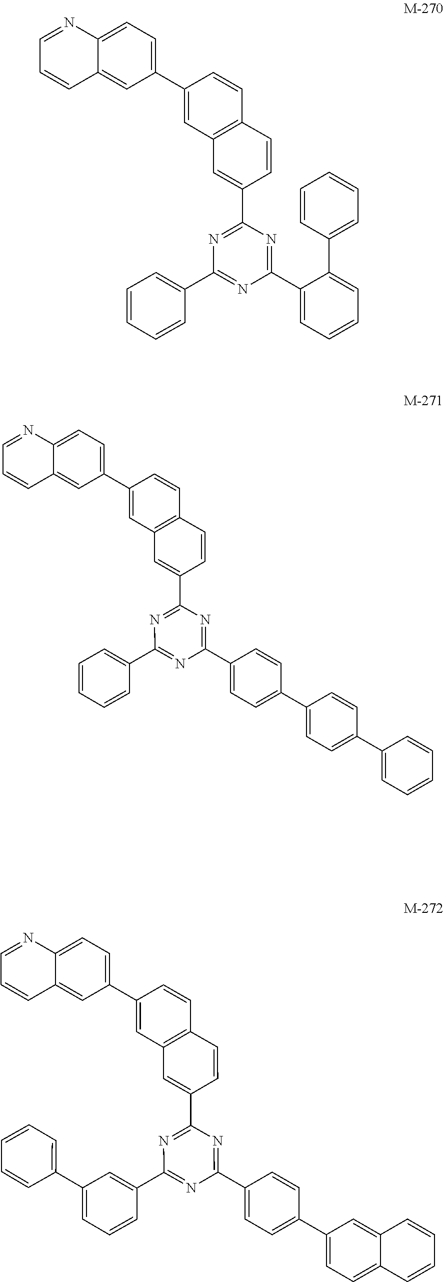 [SMILES: c1ccc(-c2nc(-c3ccc4ccc(-c5ccc6ncccc6c5)cc4c3)nc(-c3ccccc3-c3ccccc3)n2)cc1 ; c1ccc(-c2ccc(-c3ccc(-c4nc(-c5ccccc5)nc(-c5ccc6ccc(-c7ccc8ncccc8c7)cc6c5)n4)cc3)cc2)cc1 ; c1ccc(-c2cccc(-c3nc(-c4ccc(-c5ccc6ccccc6c5)cc4)nc(-c4ccc5ccc(-c6ccc7ncccc7c6)cc5c4)n3)c2)cc1]

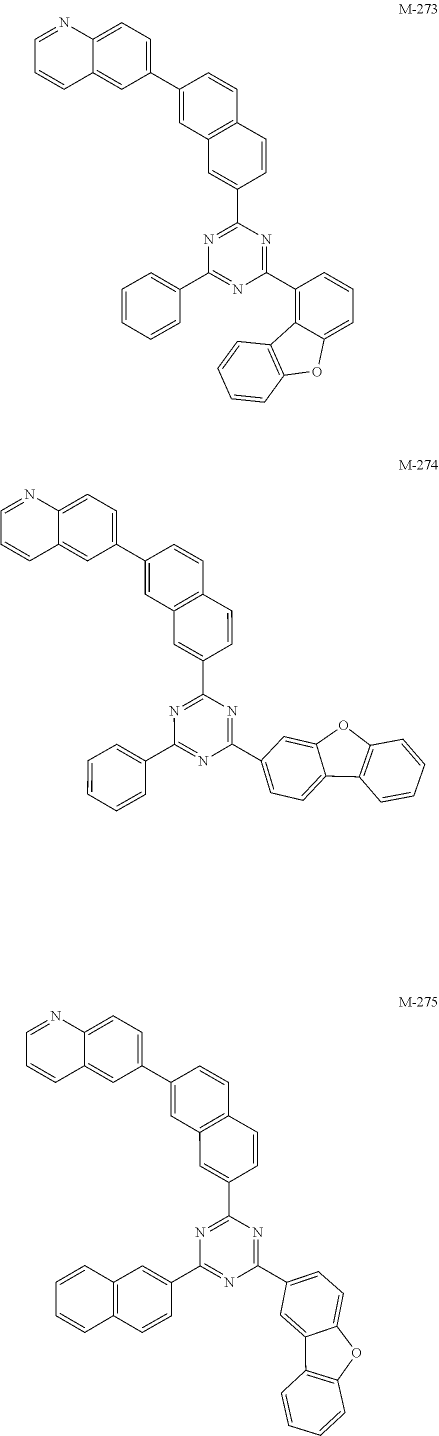 [SMILES: c1ccc(-c2nc(-c3ccc4ccc(-c5ccc6ncccc6c5)cc4c3)nc(-c3cccc4oc5ccccc5c34)n2)cc1 ; c1ccc(-c2nc(-c3ccc4ccc(-c5ccc6ncccc6c5)cc4c3)nc(-c3ccc4c(c3)oc3ccccc34)n2)cc1 ; c1ccc2cc(-c3nc(-c4ccc5ccc(-c6ccc7ncccc7c6)cc5c4)nc(-c4ccc5oc6ccccc6c5c4)n3)ccc2c1]

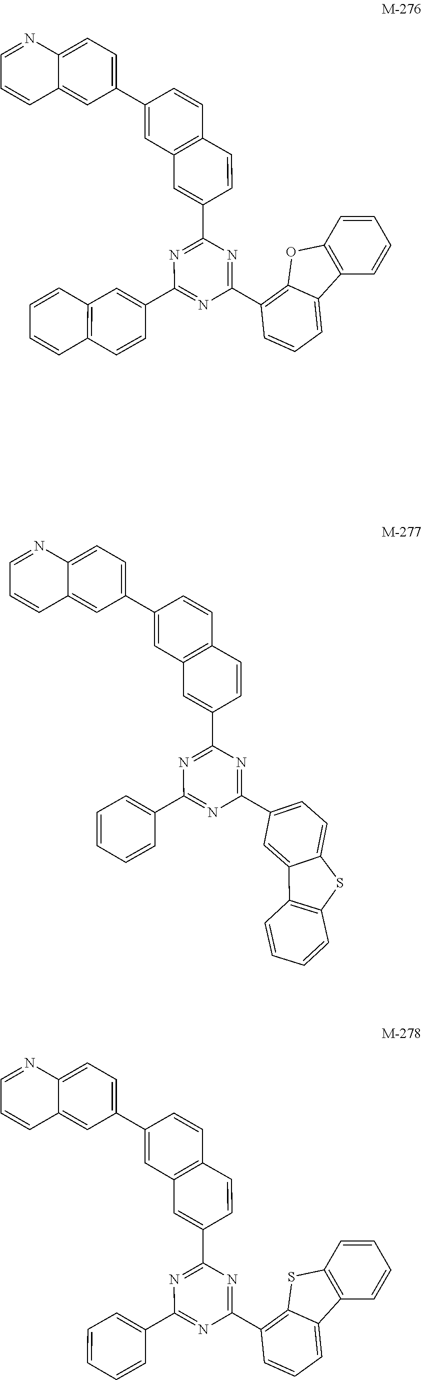 [SMILES: c1ccc2cc(-c3nc(-c4ccc5ccc(-c6ccc7ncccc7c6)cc5c4)nc(-c4cccc5c4oc4ccccc45)n3)ccc2c1 ; c1ccc(-c2nc(-c3ccc4ccc(-c5ccc6ncccc6c5)cc4c3)nc(-c3ccc4sc5ccccc5c4c3)n2)cc1 ; c1ccc(-c2nc(-c3ccc4ccc(-c5ccc6ncccc6c5)cc4c3)nc(-c3cccc4c3sc3ccccc34)n2)cc1]

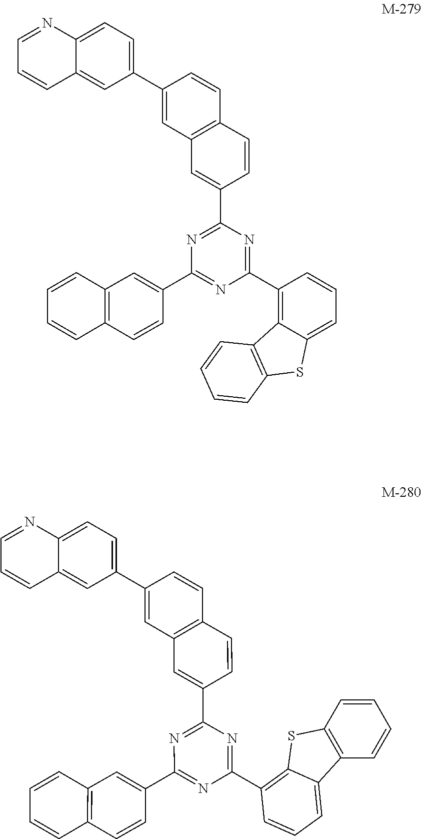 [SMILES: c1ccc2cc(-c3nc(-c4ccc5ccc(-c6ccc7ncccc7c6)cc5c4)nc(-c4cccc5sc6ccccc6c45)n3)ccc2c1 ; c1ccc2cc(-c3nc(-c4ccc5ccc(-c6ccc7ncccc7c6)cc5c4)nc(-c4cccc5c4sc4ccccc45)n3)ccc2c1]

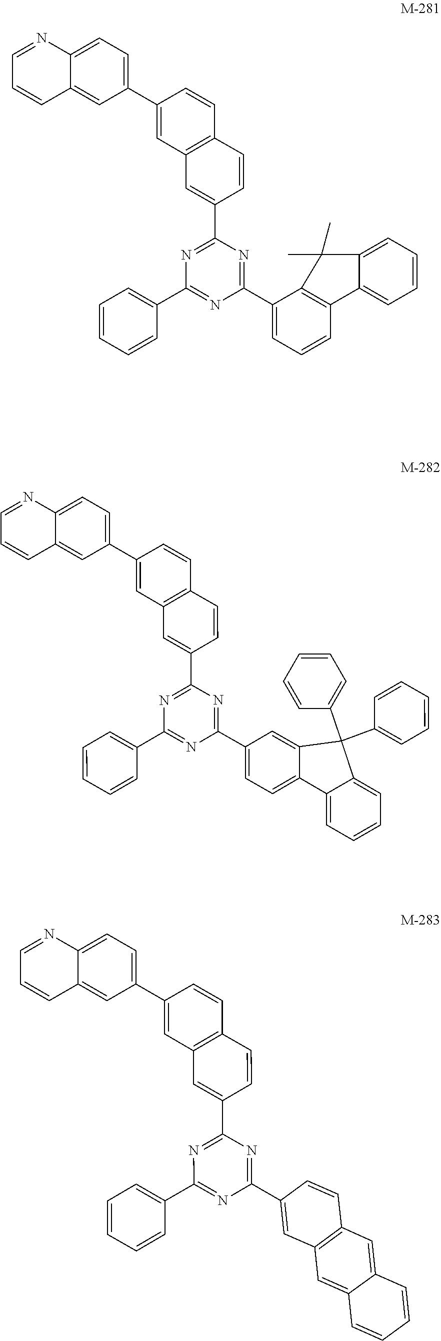 [SMILES: CC1(C)c2ccccc2-c2cccc(-c3nc(-c4ccccc4)nc(-c4ccc5ccc(-c6ccc7ncccc7c6)cc5c4)n3)c21 ; c1ccc(-c2nc(-c3ccc4c(c3)C(c3ccccc3)(c3ccccc3)c3ccccc3-4)nc(-c3ccc4ccc(-c5ccc6ncccc6c5)cc4c3)n2)cc1 ; c1ccc(-c2nc(-c3ccc4ccc(-c5ccc6ncccc6c5)cc4c3)nc(-c3ccc4cc5ccccc5cc4c3)n2)cc1]

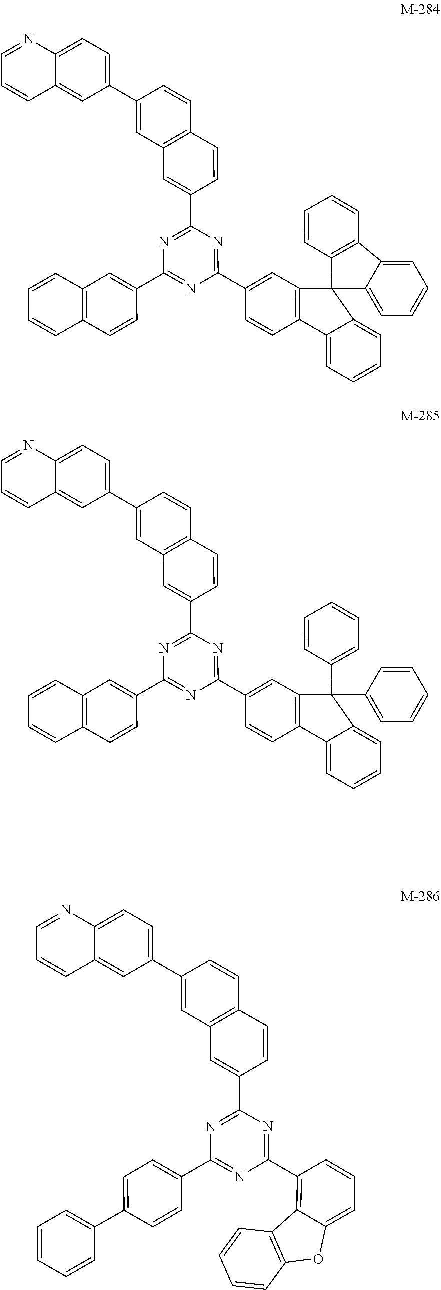 [SMILES: c1ccc2c(c1)-c1ccccc1C21c2ccccc2-c2ccc(-c3nc(-c4ccc5ccccc5c4)nc(-c4ccc5ccc(-c6ccc7ncccc7c6)cc5c4)n3)cc21 ; c1ccc(C2(c3ccccc3)c3ccccc3-c3ccc(-c4nc(-c5ccc6ccccc6c5)nc(-c5ccc6ccc(-c7ccc8ncccc8c7)cc6c5)n4)cc32)cc1 ; c1ccc(-c2ccc(-c3nc(-c4ccc5ccc(-c6ccc7ncccc7c6)cc5c4)nc(-c4cccc5oc6ccccc6c45)n3)cc2)cc1]

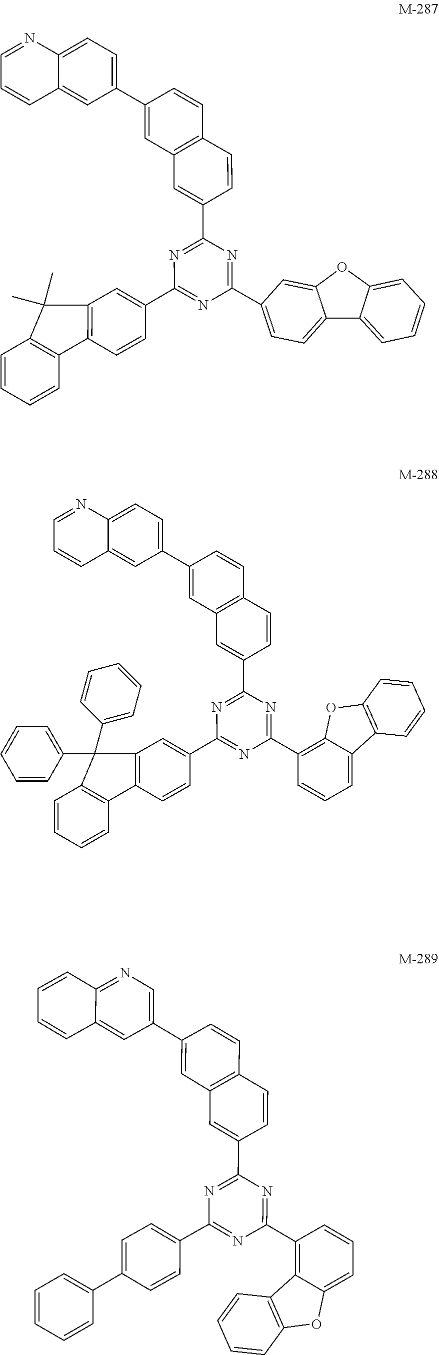 [SMILES: CC1(C)c2ccccc2-c2ccc(-c3nc(-c4ccc5ccc(-c6ccc7ncccc7c6)cc5c4)nc(-c4ccc5c(c4)oc4ccccc45)n3)cc21 ; c1ccc(C2(c3ccccc3)c3ccccc3-c3ccc(-c4nc(-c5ccc6ccc(-c7ccc8ncccc8c7)cc6c5)nc(-c5cccc6c5oc5ccccc56)n4)cc32)cc1 ; c1ccc(-c2ccc(-c3nc(-c4ccc5ccc(-c6cnc7ccccc7c6)cc5c4)nc(-c4cccc5oc6ccccc6c45)n3)cc2)cc1]

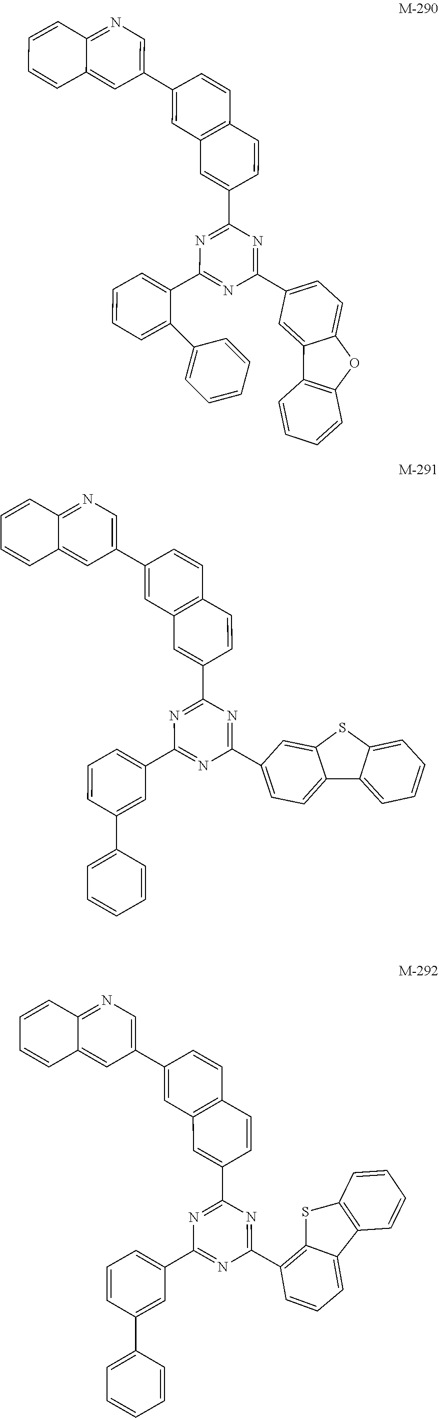 [SMILES: c1ccc(-c2ccccc2-c2nc(-c3ccc4ccc(-c5cnc6ccccc6c5)cc4c3)nc(-c3ccc4oc5ccccc5c4c3)n2)cc1 ; c1ccc(-c2cccc(-c3nc(-c4ccc5ccc(-c6cnc7ccccc7c6)cc5c4)nc(-c4ccc5c(c4)sc4ccccc45)n3)c2)cc1 ; c1ccc(-c2cccc(-c3nc(-c4ccc5ccc(-c6cnc7ccccc7c6)cc5c4)nc(-c4cccc5c4sc4ccccc45)n3)c2)cc1]

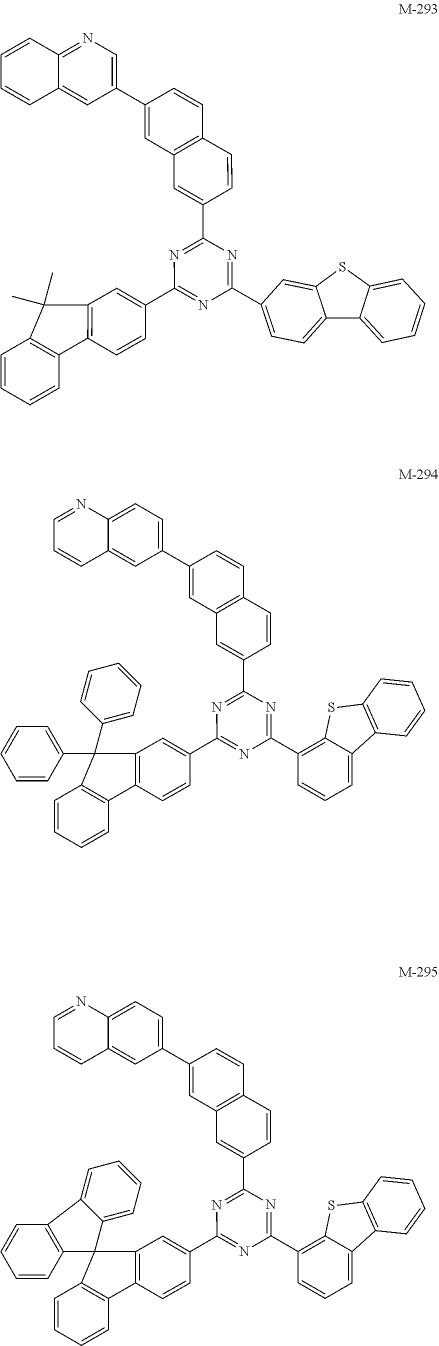 [SMILES: CC1(C)c2ccccc2-c2ccc(-c3nc(-c4ccc5ccc(-c6cnc7ccccc7c6)cc5c4)nc(-c4ccc5c(c4)sc4ccccc45)n3)cc21 ; c1ccc(C2(c3ccccc3)c3ccccc3-c3ccc(-c4nc(-c5ccc6ccc(-c7ccc8ncccc8c7)cc6c5)nc(-c5cccc6c5sc5ccccc56)n4)cc32)cc1 ; c1ccc2c(c1)-c1ccccc1C21c2ccccc2-c2ccc(-c3nc(-c4ccc5ccc(-c6ccc7ncccc7c6)cc5c4)nc(-c4cccc5c4sc4ccccc45)n3)cc21]

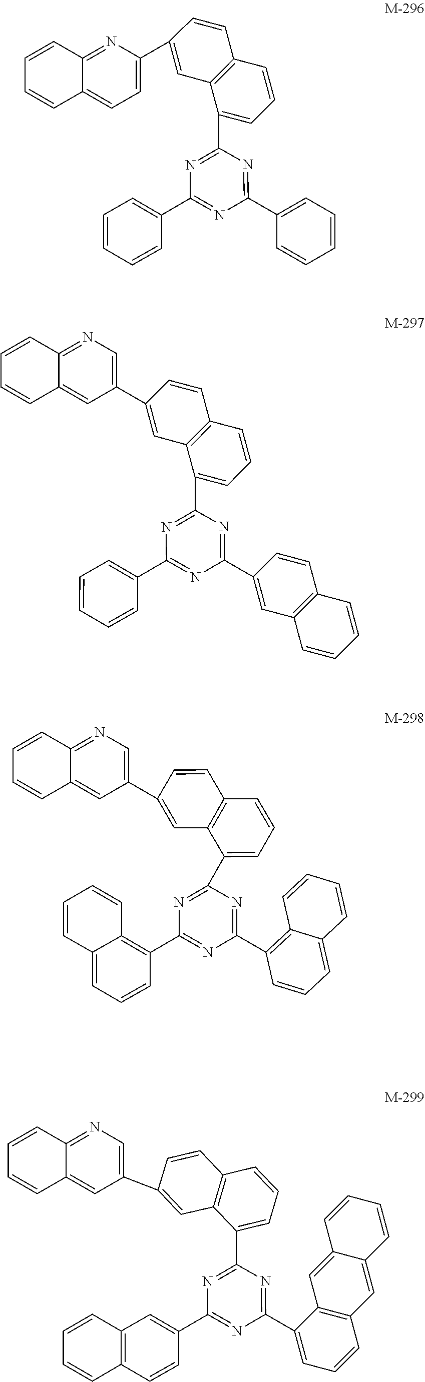 [SMILES: c1ccc(-c2nc(-c3ccccc3)nc(-c3cccc4ccc(-c5ccc6ccccc6n5)cc34)n2)cc1 ; c1ccc(-c2nc(-c3ccc4ccccc4c3)nc(-c3cccc4ccc(-c5cnc6ccccc6c5)cc34)n2)cc1 ; c1ccc2ncc(-c3ccc4cccc(-c5nc(-c6cccc7ccccc67)nc(-c6cccc7ccccc67)n5)c4c3)cc2c1 ; c1ccc2cc(-c3nc(-c4cccc5ccc(-c6cnc7ccccc7c6)cc45)nc(-c4cccc5cc6ccccc6cc45)n3)ccc2c1]

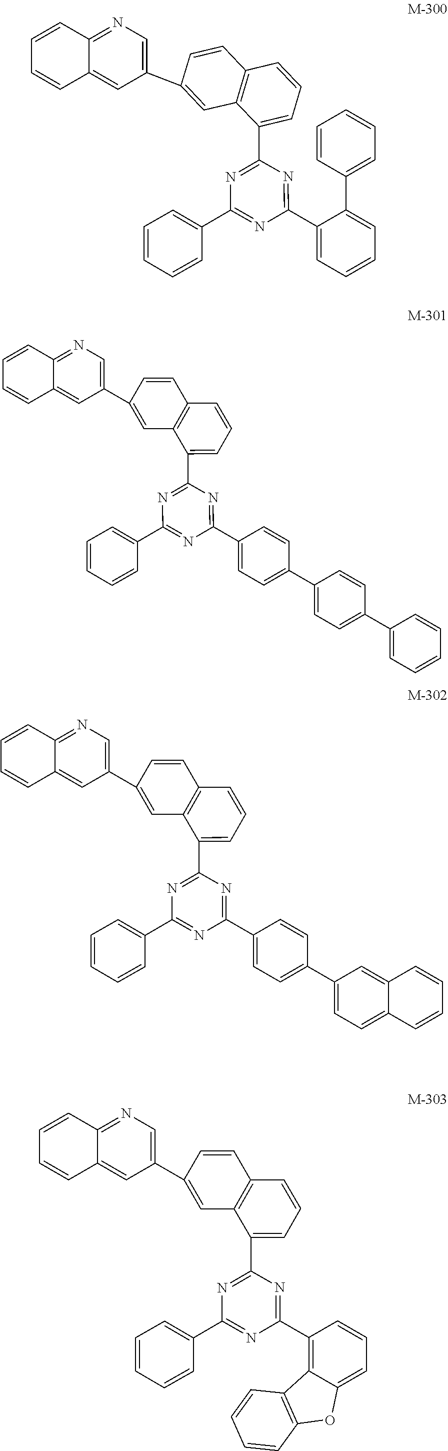 [SMILES: c1ccc(-c2nc(-c3ccccc3-c3ccccc3)nc(-c3cccc4ccc(-c5cnc6ccccc6c5)cc34)n2)cc1 ; c1ccc(-c2ccc(-c3ccc(-c4nc(-c5ccccc5)nc(-c5cccc6ccc(-c7cnc8ccccc8c7)cc56)n4)cc3)cc2)cc1 ; c1ccc(-c2nc(-c3ccc(-c4ccc5ccccc5c4)cc3)nc(-c3cccc4ccc(-c5cnc6ccccc6c5)cc34)n2)cc1 ; c1ccc(-c2nc(-c3cccc4ccc(-c5cnc6ccccc6c5)cc34)nc(-c3cccc4oc5ccccc5c34)n2)cc1]

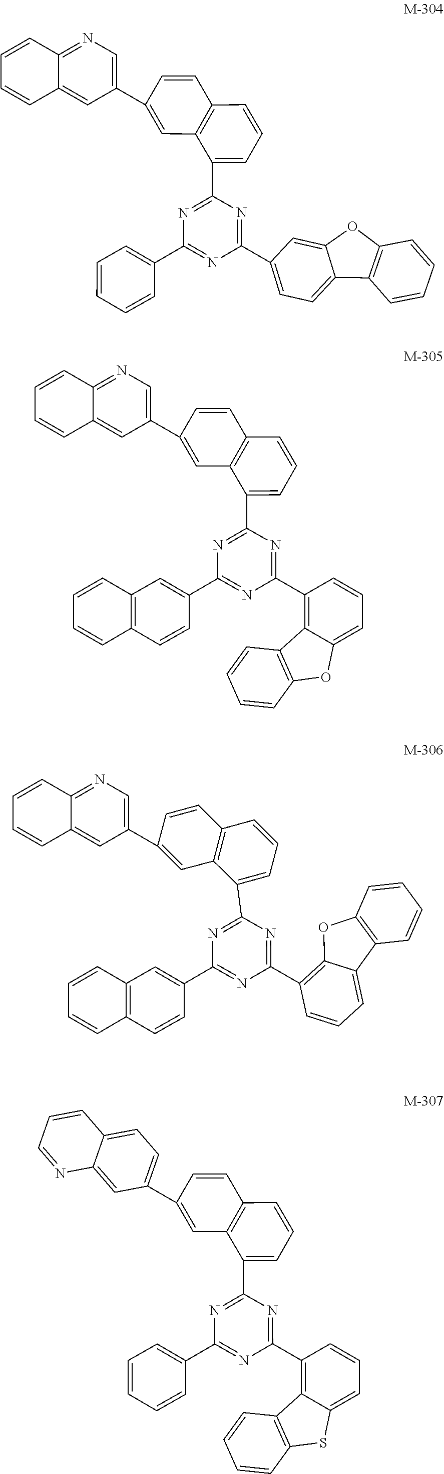 [SMILES: c1ccc(-c2nc(-c3ccc4c(c3)oc3ccccc34)nc(-c3cccc4ccc(-c5cnc6ccccc6c5)cc34)n2)cc1 ; c1ccc2cc(-c3nc(-c4cccc5ccc(-c6cnc7ccccc7c6)cc45)nc(-c4cccc5oc6ccccc6c45)n3)ccc2c1 ; c1ccc2cc(-c3nc(-c4cccc5ccc(-c6cnc7ccccc7c6)cc45)nc(-c4cccc5c4oc4ccccc45)n3)ccc2c1 ; c1ccc(-c2nc(-c3cccc4ccc(-c5ccc6cccnc6c5)cc34)nc(-c3cccc4sc5ccccc5c34)n2)cc1]

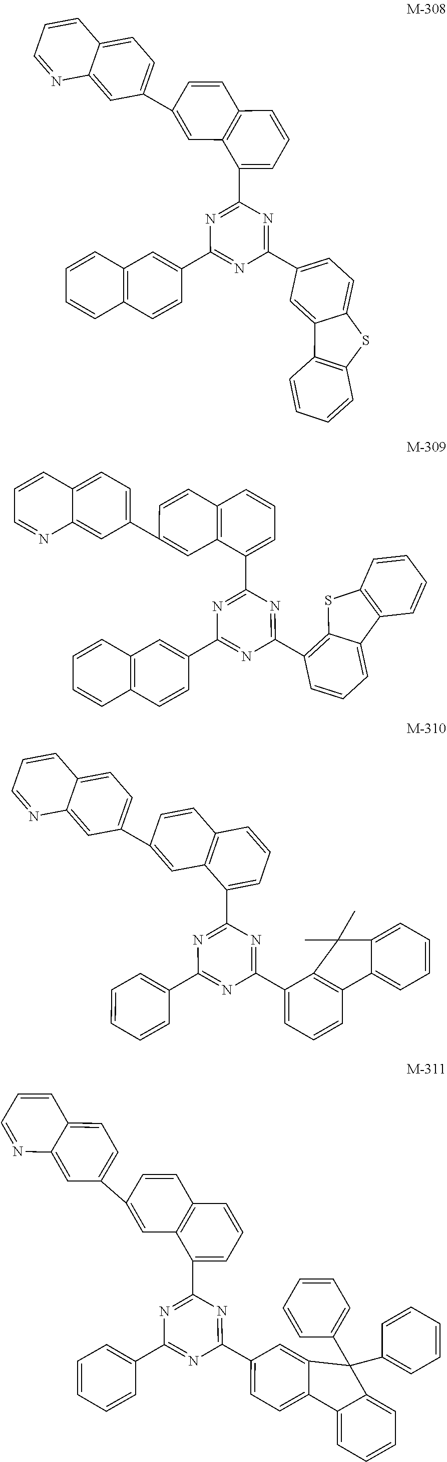 [SMILES: c1ccc2cc(-c3nc(-c4ccc5sc6ccccc6c5c4)nc(-c4cccc5ccc(-c6ccc7cccnc7c6)cc45)n3)ccc2c1 ; c1ccc2cc(-c3nc(-c4cccc5ccc(-c6ccc7cccnc7c6)cc45)nc(-c4cccc5c4sc4ccccc45)n3)ccc2c1 ; CC1(C)c2ccccc2-c2cccc(-c3nc(-c4ccccc4)nc(-c4cccc5ccc(-c6ccc7cccnc7c6)cc45)n3)c21 ; c1ccc(-c2nc(-c3ccc4c(c3)C(c3ccccc3)(c3ccccc3)c3ccccc3-4)nc(-c3cccc4ccc(-c5ccc6cccnc6c5)cc34)n2)cc1]

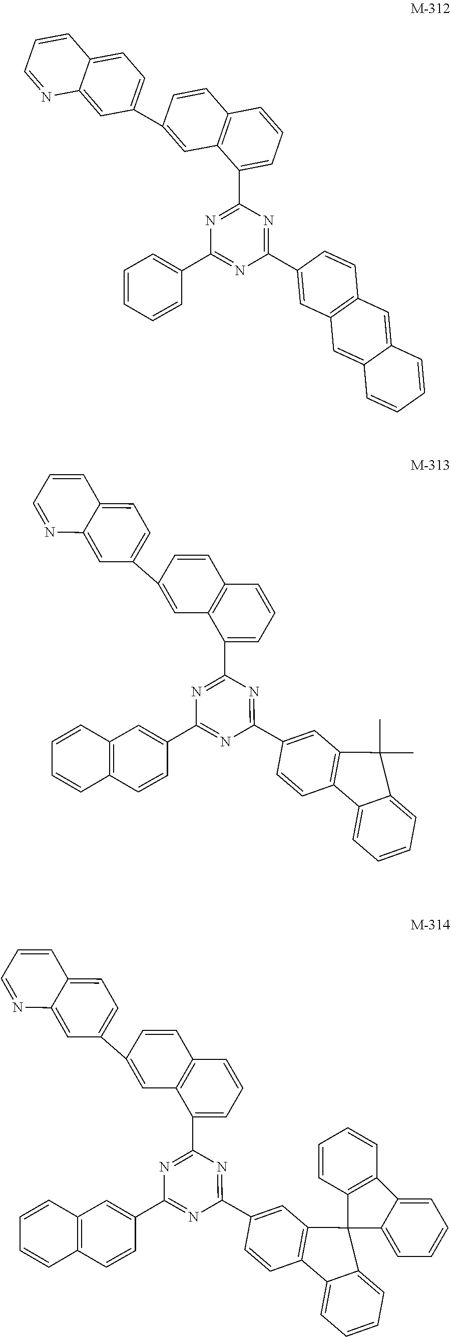 [SMILES: c1ccc(-c2nc(-c3ccc4cc5ccccc5cc4c3)nc(-c3cccc4ccc(-c5ccc6cccnc6c5)cc34)n2)cc1 ; CC1(C)c2ccccc2-c2ccc(-c3nc(-c4ccc5ccccc5c4)nc(-c4cccc5ccc(-c6ccc7cccnc7c6)cc45)n3)cc21 ; c1ccc2c(c1)-c1ccccc1C21c2ccccc2-c2ccc(-c3nc(-c4ccc5ccccc5c4)nc(-c4cccc5ccc(-c6ccc7cccnc7c6)cc45)n3)cc21]

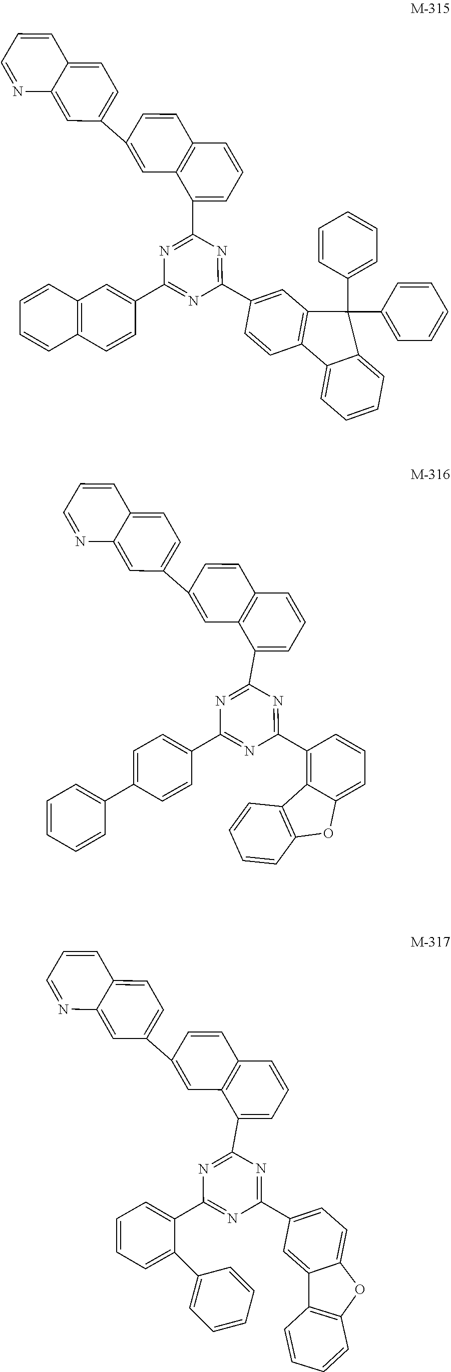 [SMILES: c1ccc(C2(c3ccccc3)c3ccccc3-c3ccc(-c4nc(-c5ccc6ccccc6c5)nc(-c5cccc6ccc(-c7ccc8cccnc8c7)cc56)n4)cc32)cc1 ; c1ccc(-c2ccc(-c3nc(-c4cccc5ccc(-c6ccc7cccnc7c6)cc45)nc(-c4cccc5oc6ccccc6c45)n3)cc2)cc1 ; c1ccc(-c2ccccc2-c2nc(-c3ccc4oc5ccccc5c4c3)nc(-c3cccc4ccc(-c5ccc6cccnc6c5)cc34)n2)cc1]

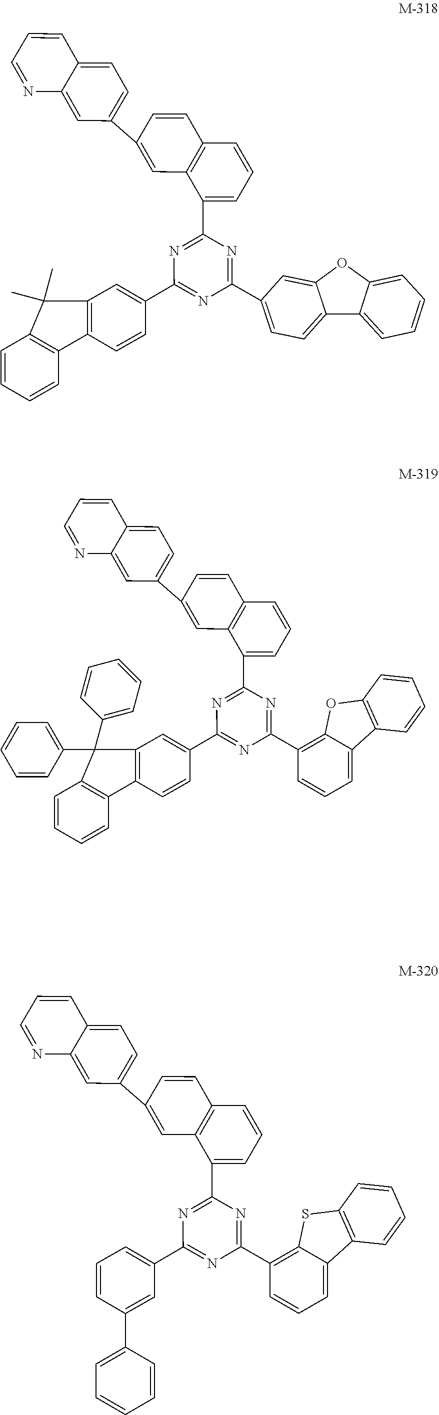 [SMILES: CC1(C)c2ccccc2-c2ccc(-c3nc(-c4ccc5c(c4)oc4ccccc45)nc(-c4cccc5ccc(-c6ccc7cccnc7c6)cc45)n3)cc21 ; c1ccc(C2(c3ccccc3)c3ccccc3-c3ccc(-c4nc(-c5cccc6ccc(-c7ccc8cccnc8c7)cc56)nc(-c5cccc6c5oc5ccccc56)n4)cc32)cc1 ; c1ccc(-c2cccc(-c3nc(-c4cccc5ccc(-c6ccc7cccnc7c6)cc45)nc(-c4cccc5c4sc4ccccc45)n3)c2)cc1]

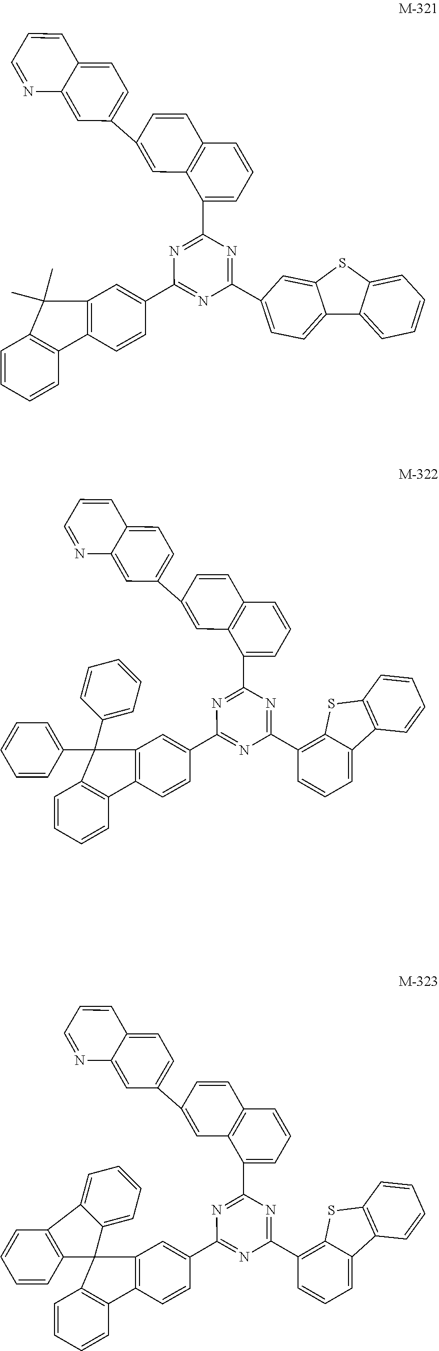 [SMILES: CC1(C)c2ccccc2-c2ccc(-c3nc(-c4ccc5c(c4)sc4ccccc45)nc(-c4cccc5ccc(-c6ccc7cccnc7c6)cc45)n3)cc21 ; c1ccc(C2(c3ccccc3)c3ccccc3-c3ccc(-c4nc(-c5cccc6ccc(-c7ccc8cccnc8c7)cc56)nc(-c5cccc6c5sc5ccccc56)n4)cc32)cc1 ; c1ccc2c(c1)-c1ccccc1C21c2ccccc2-c2ccc(-c3nc(-c4cccc5ccc(-c6ccc7cccnc7c6)cc45)nc(-c4cccc5c4sc4ccccc45)n3)cc21]

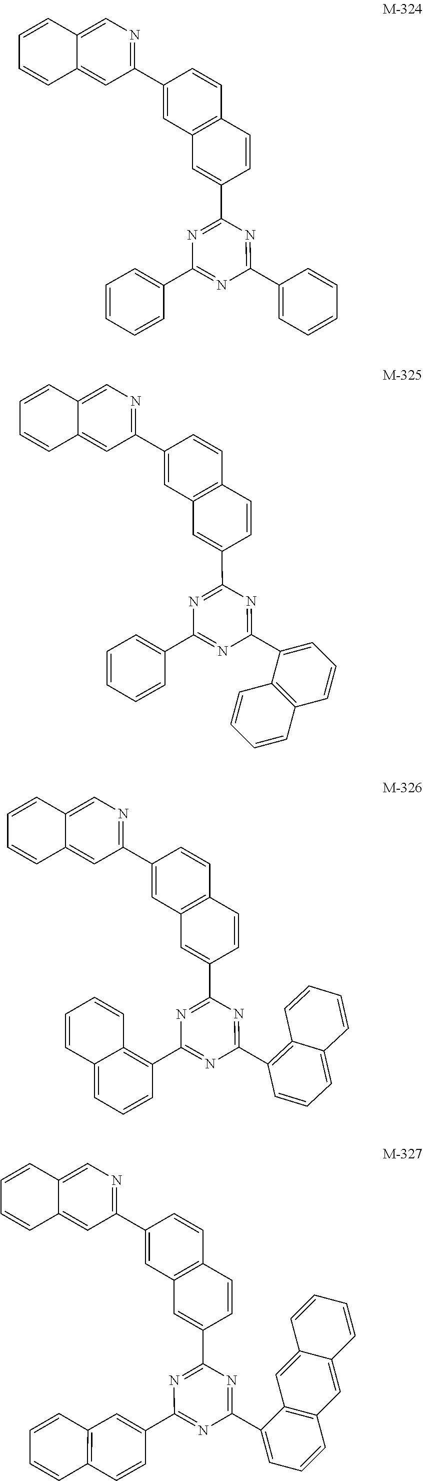 [SMILES: c1ccc(-c2nc(-c3ccccc3)nc(-c3ccc4ccc(-c5cc6ccccc6cn5)cc4c3)n2)cc1 ; c1ccc(-c2nc(-c3ccc4ccc(-c5cc6ccccc6cn5)cc4c3)nc(-c3cccc4ccccc34)n2)cc1 ; c1ccc2cc(-c3ccc4ccc(-c5nc(-c6cccc7ccccc67)nc(-c6cccc7ccccc67)n5)cc4c3)ncc2c1 ; c1ccc2cc(-c3nc(-c4ccc5ccc(-c6cc7ccccc7cn6)cc5c4)nc(-c4cccc5cc6ccccc6cc45)n3)ccc2c1]

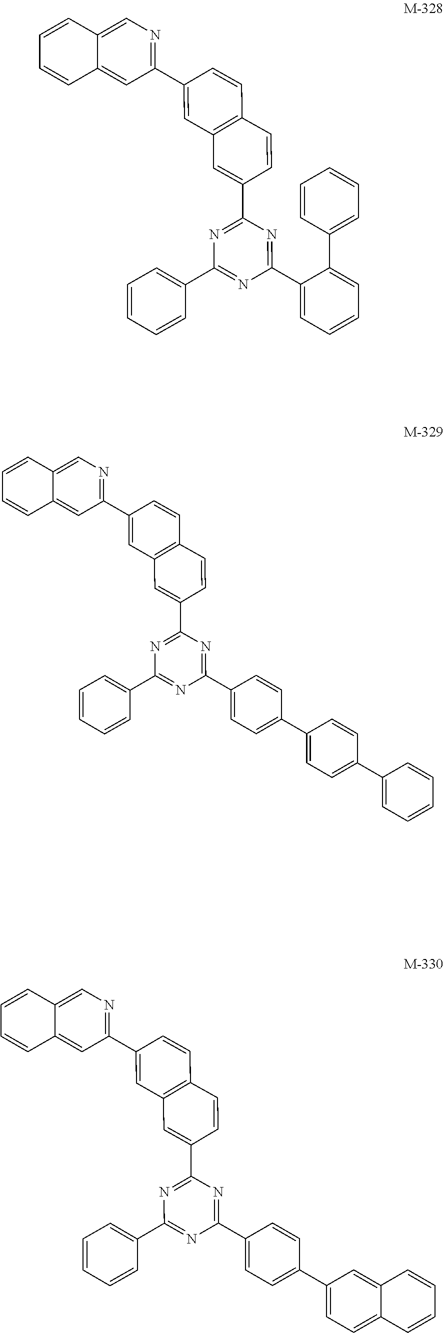 [SMILES: c1ccc(-c2nc(-c3ccc4ccc(-c5cc6ccccc6cn5)cc4c3)nc(-c3ccccc3-c3ccccc3)n2)cc1 ; c1ccc(-c2ccc(-c3ccc(-c4nc(-c5ccccc5)nc(-c5ccc6ccc(-c7cc8ccccc8cn7)cc6c5)n4)cc3)cc2)cc1 ; c1ccc(-c2nc(-c3ccc(-c4ccc5ccccc5c4)cc3)nc(-c3ccc4ccc(-c5cc6ccccc6cn5)cc4c3)n2)cc1]

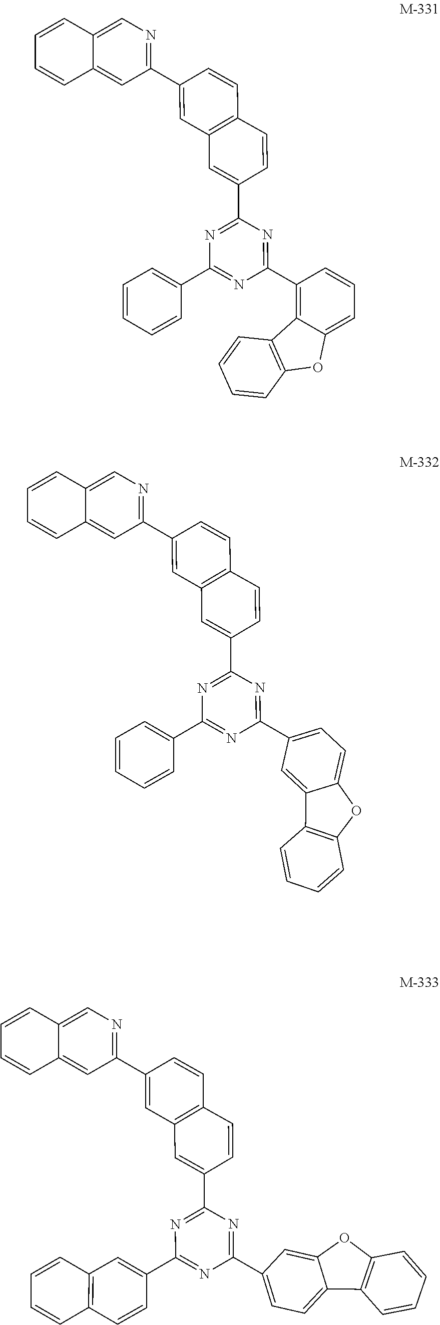 [SMILES: c1ccc(-c2nc(-c3ccc4ccc(-c5cc6ccccc6cn5)cc4c3)nc(-c3cccc4oc5ccccc5c34)n2)cc1 ; c1ccc(-c2nc(-c3ccc4ccc(-c5cc6ccccc6cn5)cc4c3)nc(-c3ccc4oc5ccccc5c4c3)n2)cc1 ; c1ccc2cc(-c3nc(-c4ccc5ccc(-c6cc7ccccc7cn6)cc5c4)nc(-c4ccc5c(c4)oc4ccccc45)n3)ccc2c1]

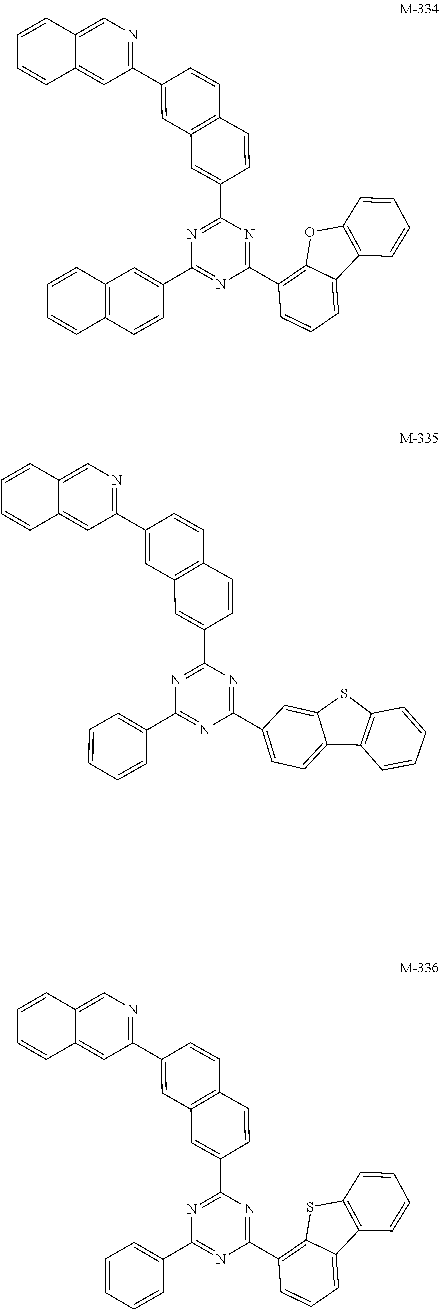 [SMILES: c1ccc2cc(-c3nc(-c4ccc5ccc(-c6cc7ccccc7cn6)cc5c4)nc(-c4cccc5c4oc4ccccc45)n3)ccc2c1 ; c1ccc(-c2nc(-c3ccc4ccc(-c5cc6ccccc6cn5)cc4c3)nc(-c3ccc4c(c3)sc3ccccc34)n2)cc1 ; c1ccc(-c2nc(-c3ccc4ccc(-c5cc6ccccc6cn5)cc4c3)nc(-c3cccc4c3sc3ccccc34)n2)cc1]

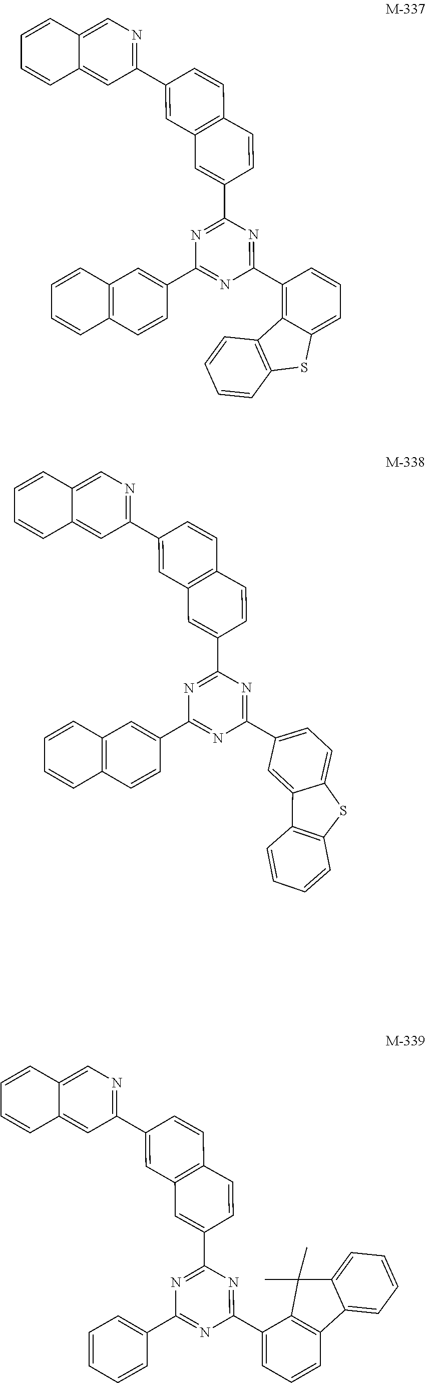 [SMILES: c1ccc2cc(-c3nc(-c4ccc5ccc(-c6cc7ccccc7cn6)cc5c4)nc(-c4cccc5sc6ccccc6c45)n3)ccc2c1 ; c1ccc2cc(-c3nc(-c4ccc5ccc(-c6cc7ccccc7cn6)cc5c4)nc(-c4ccc5sc6ccccc6c5c4)n3)ccc2c1 ; CC1(C)c2ccccc2-c2cccc(-c3nc(-c4ccccc4)nc(-c4ccc5ccc(-c6cc7ccccc7cn6)cc5c4)n3)c21]

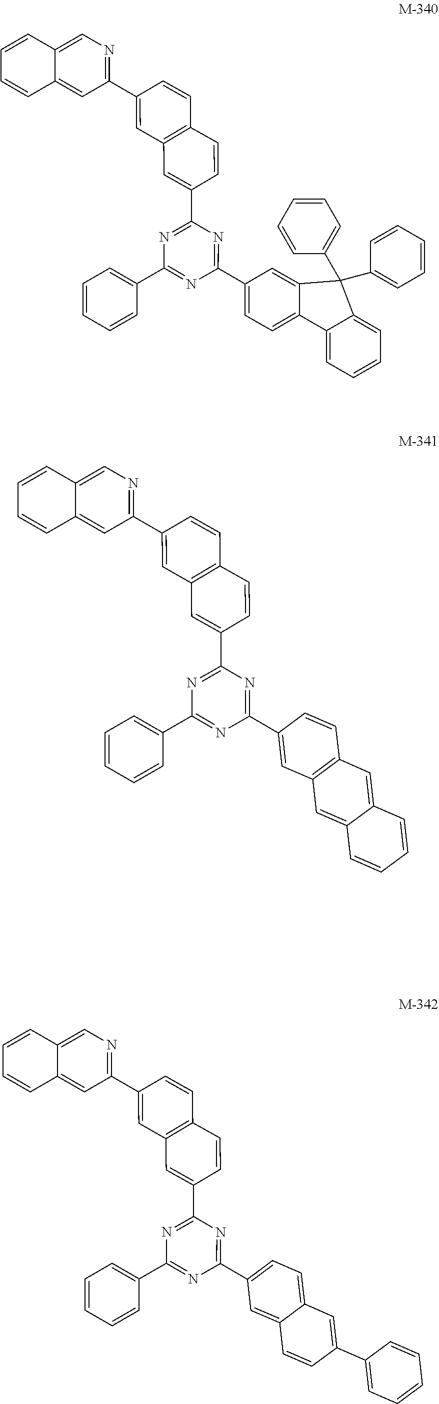 [SMILES: c1ccc(-c2nc(-c3ccc4c(c3)C(c3ccccc3)(c3ccccc3)c3ccccc3-4)nc(-c3ccc4ccc(-c5cc6ccccc6cn5)cc4c3)n2)cc1 ; c1ccc(-c2nc(-c3ccc4ccc(-c5cc6ccccc6cn5)cc4c3)nc(-c3ccc4cc5ccccc5cc4c3)n2)cc1 ; c1ccc(-c2ccc3cc(-c4nc(-c5ccccc5)nc(-c5ccc6ccc(-c7cc8ccccc8cn7)cc6c5)n4)ccc3c2)cc1]

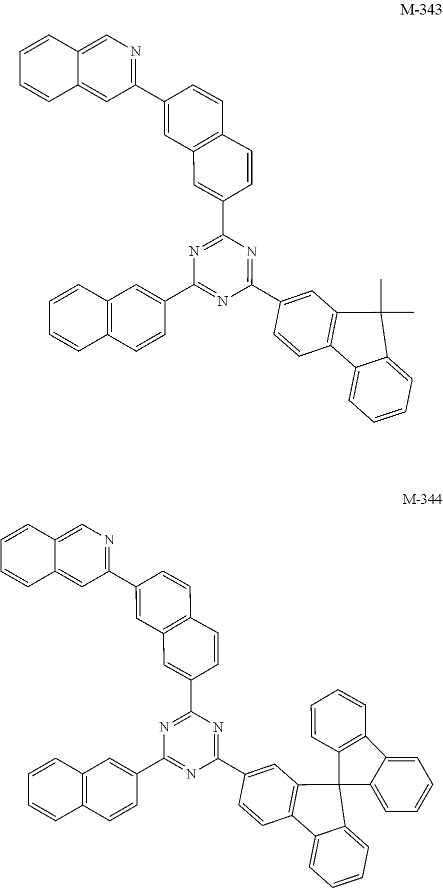 [SMILES: CC1(C)c2ccccc2-c2ccc(-c3nc(-c4ccc5ccccc5c4)nc(-c4ccc5ccc(-c6cc7ccccc7cn6)cc5c4)n3)cc21 ; c1ccc2c(c1)-c1ccccc1C21c2ccccc2-c2ccc(-c3nc(-c4ccc5ccccc5c4)nc(-c4ccc5ccc(-c6cc7ccccc7cn6)cc5c4)n3)cc21]

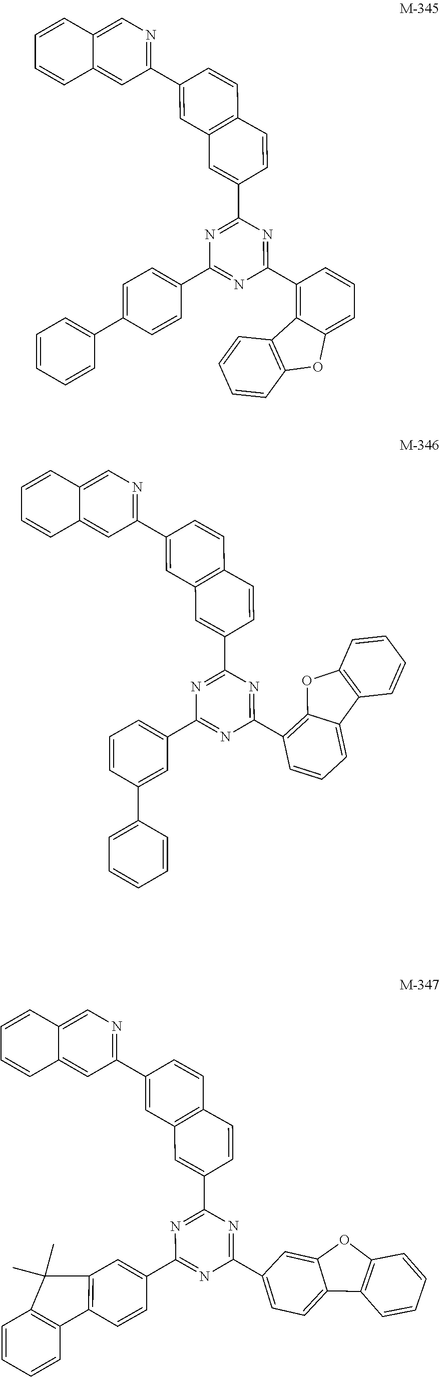 [SMILES: c1ccc(-c2ccc(-c3nc(-c4ccc5ccc(-c6cc7ccccc7cn6)cc5c4)nc(-c4cccc5oc6ccccc6c45)n3)cc2)cc1 ; c1ccc(-c2cccc(-c3nc(-c4ccc5ccc(-c6cc7ccccc7cn6)cc5c4)nc(-c4cccc5c4oc4ccccc45)n3)c2)cc1 ; CC1(C)c2ccccc2-c2ccc(-c3nc(-c4ccc5ccc(-c6cc7ccccc7cn6)cc5c4)nc(-c4ccc5c(c4)oc4ccccc45)n3)cc21]

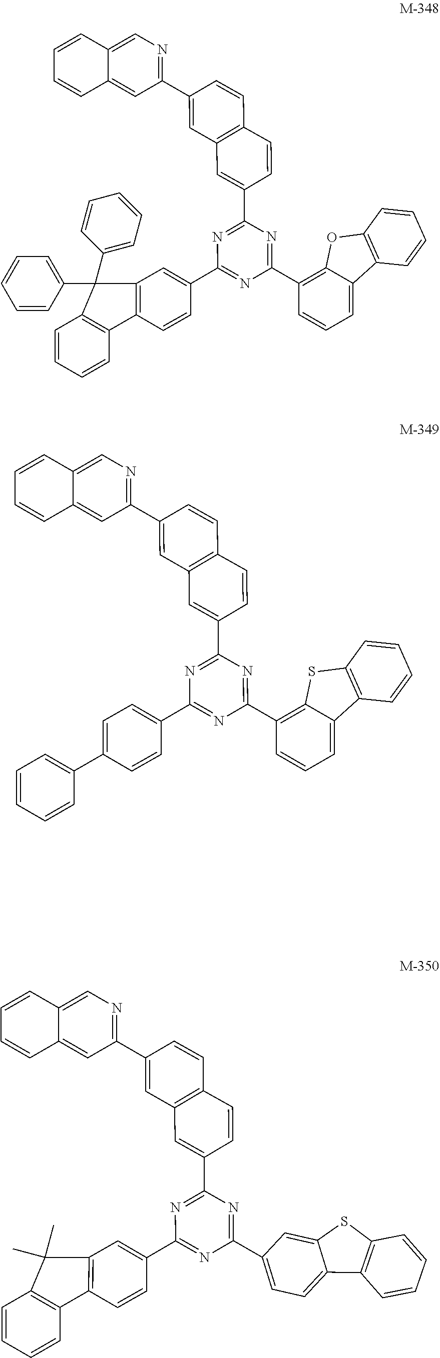 [SMILES: c1ccc(C2(c3ccccc3)c3ccccc3-c3ccc(-c4nc(-c5ccc6ccc(-c7cc8ccccc8cn7)cc6c5)nc(-c5cccc6c5oc5ccccc56)n4)cc32)cc1 ; c1ccc(-c2ccc(-c3nc(-c4ccc5ccc(-c6cc7ccccc7cn6)cc5c4)nc(-c4cccc5c4sc4ccccc45)n3)cc2)cc1 ; CC1(C)c2ccccc2-c2ccc(-c3nc(-c4ccc5ccc(-c6cc7ccccc7cn6)cc5c4)nc(-c4ccc5c(c4)sc4ccccc45)n3)cc21]

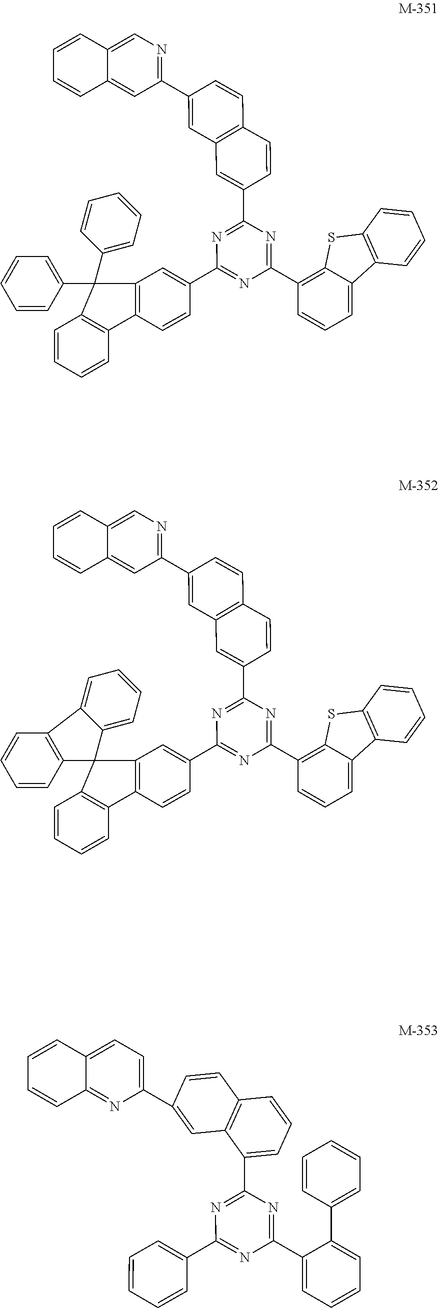 [SMILES: c1ccc(C2(c3ccccc3)c3ccccc3-c3ccc(-c4nc(-c5ccc6ccc(-c7cc8ccccc8cn7)cc6c5)nc(-c5cccc6c5sc5ccccc56)n4)cc32)cc1 ; c1ccc2c(c1)-c1ccccc1C21c2ccccc2-c2ccc(-c3nc(-c4ccc5ccc(-c6cc7ccccc7cn6)cc5c4)nc(-c4cccc5c4sc4ccccc45)n3)cc21 ; c1ccc(-c2nc(-c3ccccc3-c3ccccc3)nc(-c3cccc4ccc(-c5ccc6ccccc6n5)cc34)n2)cc1]

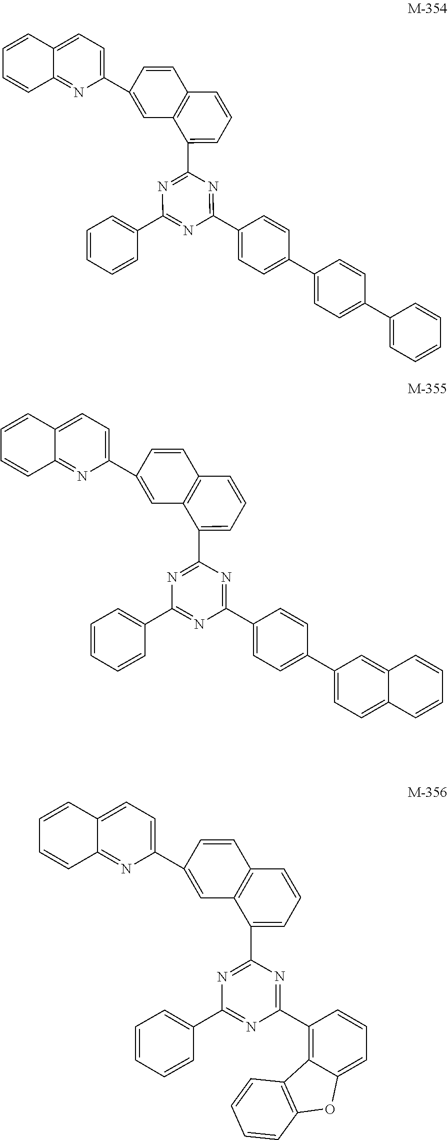 [SMILES: c1ccc(-c2ccc(-c3ccc(-c4nc(-c5ccccc5)nc(-c5cccc6ccc(-c7ccc8ccccc8n7)cc56)n4)cc3)cc2)cc1 ; c1ccc(-c2nc(-c3ccc(-c4ccc5ccccc5c4)cc3)nc(-c3cccc4ccc(-c5ccc6ccccc6n5)cc34)n2)cc1 ; c1ccc(-c2nc(-c3cccc4ccc(-c5ccc6ccccc6n5)cc34)nc(-c3cccc4oc5ccccc5c34)n2)cc1]

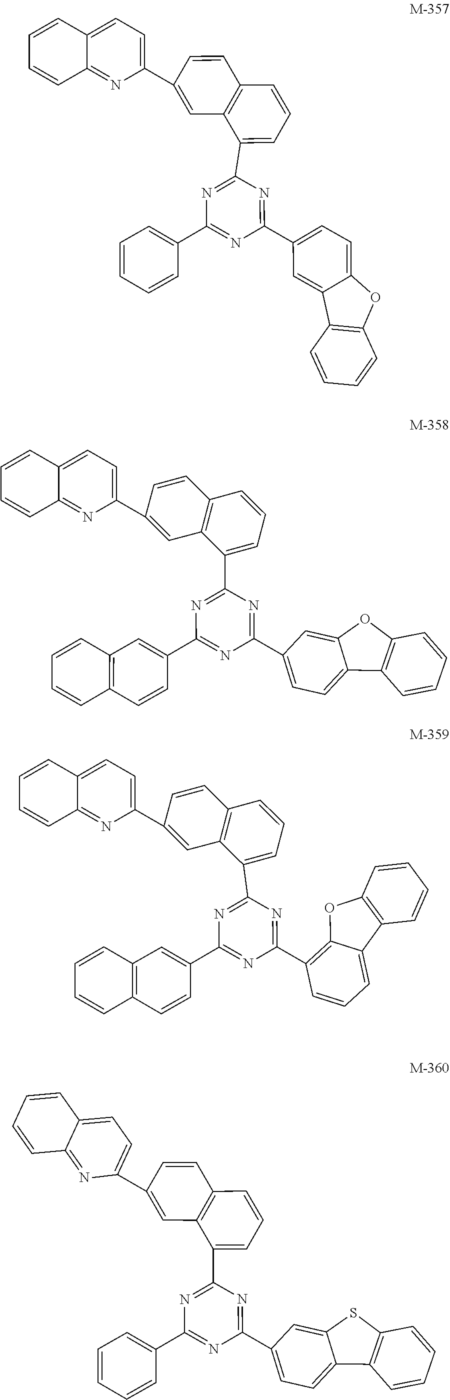 [SMILES: c1ccc(-c2nc(-c3ccc4oc5ccccc5c4c3)nc(-c3cccc4ccc(-c5ccc6ccccc6n5)cc34)n2)cc1 ; c1ccc2cc(-c3nc(-c4ccc5c(c4)oc4ccccc45)nc(-c4cccc5ccc(-c6ccc7ccccc7n6)cc45)n3)ccc2c1 ; c1ccc2cc(-c3nc(-c4cccc5ccc(-c6ccc7ccccc7n6)cc45)nc(-c4cccc5c4oc4ccccc45)n3)ccc2c1 ; c1ccc(-c2nc(-c3ccc4c(c3)sc3ccccc34)nc(-c3cccc4ccc(-c5ccc6ccccc6n5)cc34)n2)cc1]

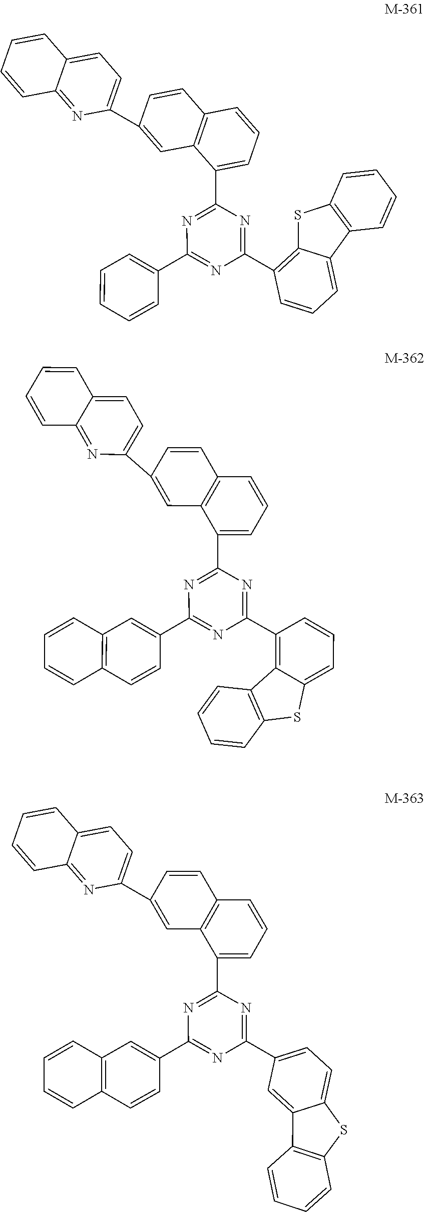 [SMILES: c1ccc(-c2nc(-c3cccc4ccc(-c5ccc6ccccc6n5)cc34)nc(-c3cccc4c3sc3ccccc34)n2)cc1 ; c1ccc2cc(-c3nc(-c4cccc5ccc(-c6ccc7ccccc7n6)cc45)nc(-c4cccc5sc6ccccc6c45)n3)ccc2c1 ; c1ccc2cc(-c3nc(-c4ccc5sc6ccccc6c5c4)nc(-c4cccc5ccc(-c6ccc7ccccc7n6)cc45)n3)ccc2c1]

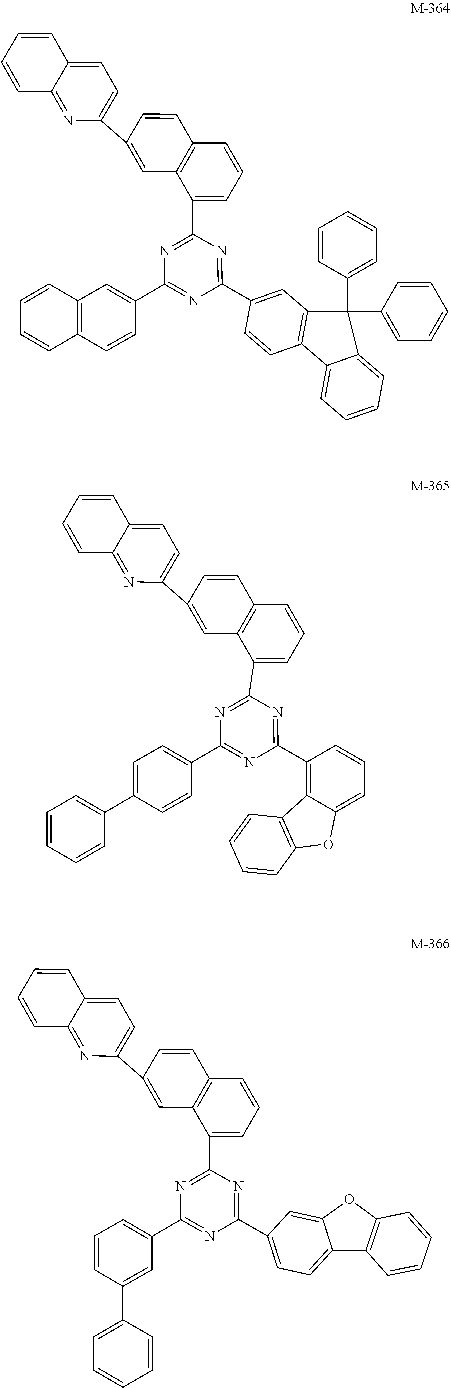 [SMILES: c1ccc(C2(c3ccccc3)c3ccccc3-c3ccc(-c4nc(-c5ccc6ccccc6c5)nc(-c5cccc6ccc(-c7ccc8ccccc8n7)cc56)n4)cc32)cc1 ; c1ccc(-c2ccc(-c3nc(-c4cccc5ccc(-c6ccc7ccccc7n6)cc45)nc(-c4cccc5oc6ccccc6c45)n3)cc2)cc1 ; c1ccc(-c2cccc(-c3nc(-c4ccc5c(c4)oc4ccccc45)nc(-c4cccc5ccc(-c6ccc7ccccc7n6)cc45)n3)c2)cc1]

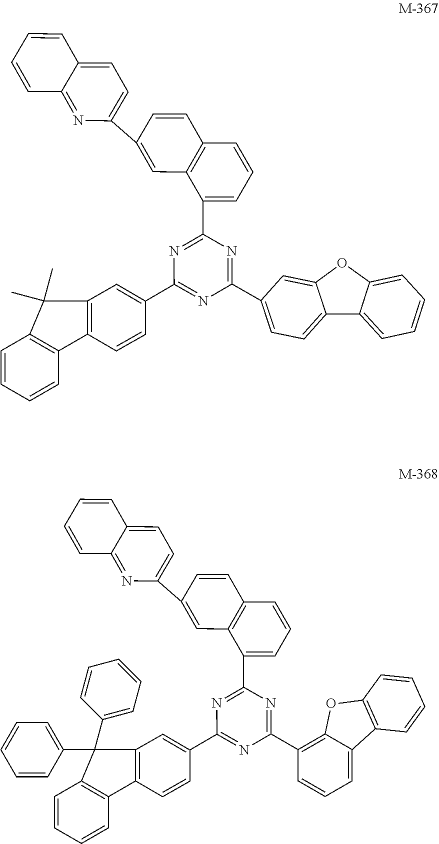 [SMILES: CC1(C)c2ccccc2-c2ccc(-c3nc(-c4ccc5c(c4)oc4ccccc45)nc(-c4cccc5ccc(-c6ccc7ccccc7n6)cc45)n3)cc21 ; c1ccc(C2(c3ccccc3)c3ccccc3-c3ccc(-c4nc(-c5cccc6ccc(-c7ccc8ccccc8n7)cc56)nc(-c5cccc6c5oc5ccccc56)n4)cc32)cc1]

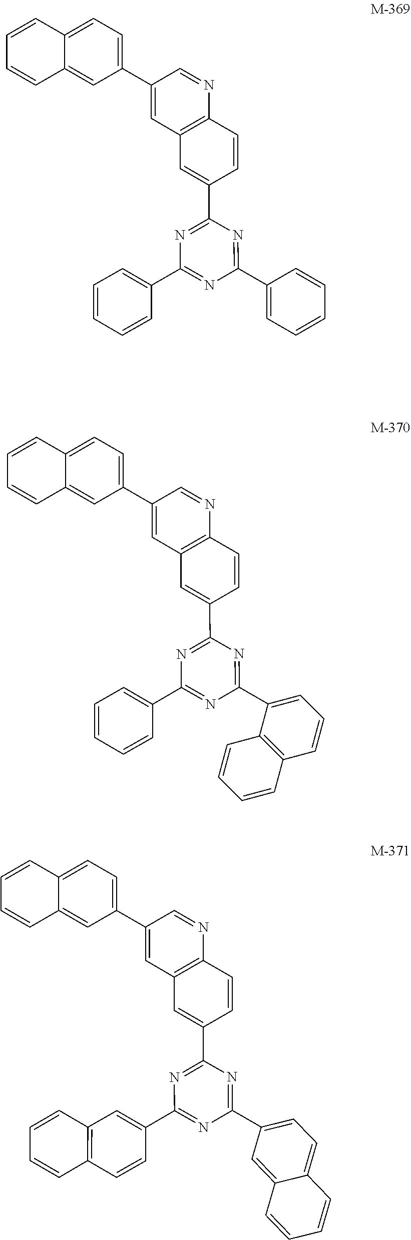 [SMILES: c1ccc(-c2nc(-c3ccccc3)nc(-c3ccc4ncc(-c5ccc6ccccc6c5)cc4c3)n2)cc1 ; c1ccc(-c2nc(-c3ccc4ncc(-c5ccc6ccccc6c5)cc4c3)nc(-c3cccc4ccccc34)n2)cc1 ; c1ccc2cc(-c3cnc4ccc(-c5nc(-c6ccc7ccccc7c6)nc(-c6ccc7ccccc7c6)n5)cc4c3)ccc2c1]

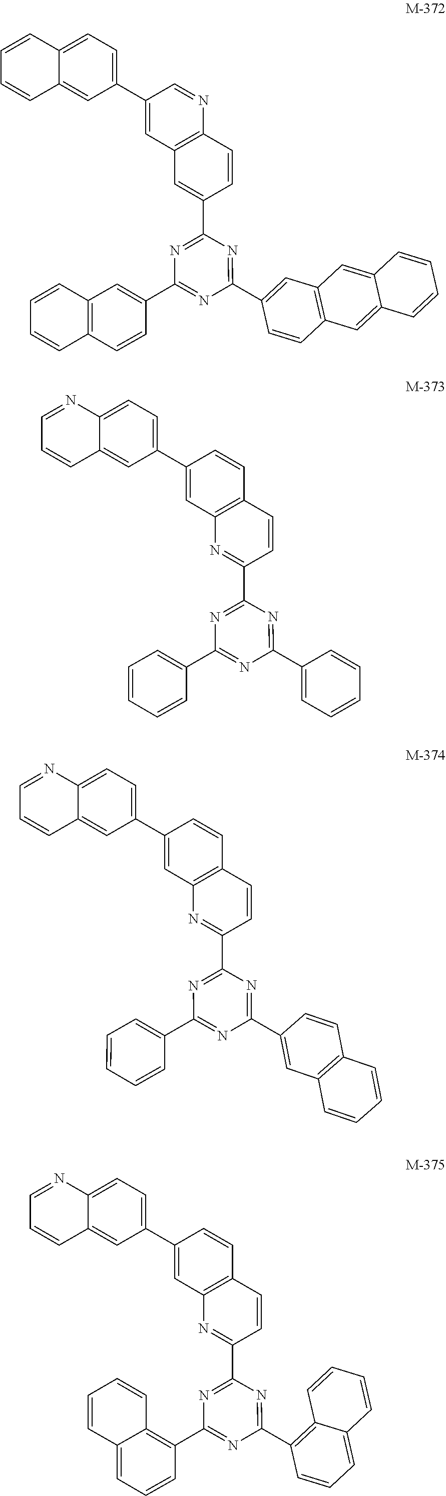 [SMILES: c1ccc2cc(-c3cnc4ccc(-c5nc(-c6ccc7ccccc7c6)nc(-c6ccc7cc8ccccc8cc7c6)n5)cc4c3)ccc2c1 ; c1ccc(-c2nc(-c3ccccc3)nc(-c3ccc4ccc(-c5ccc6ncccc6c5)cc4n3)n2)cc1 ; c1ccc(-c2nc(-c3ccc4ccccc4c3)nc(-c3ccc4ccc(-c5ccc6ncccc6c5)cc4n3)n2)cc1 ; c1cnc2ccc(-c3ccc4ccc(-c5nc(-c6cccc7ccccc67)nc(-c6cccc7ccccc67)n5)nc4c3)cc2c1]

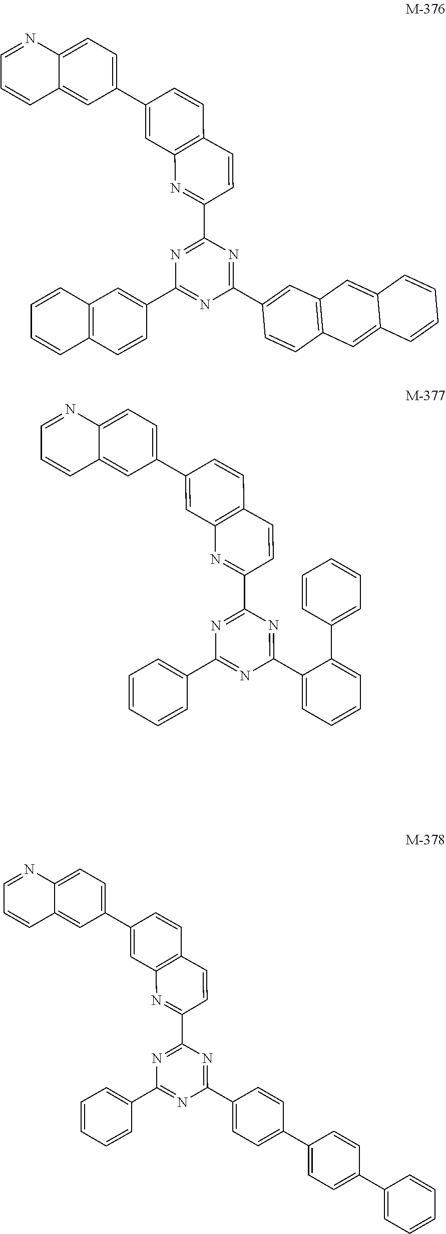 [SMILES: c1ccc2cc(-c3nc(-c4ccc5cc6ccccc6cc5c4)nc(-c4ccc5ccc(-c6ccc7ncccc7c6)cc5n4)n3)ccc2c1 ; c1ccc(-c2nc(-c3ccc4ccc(-c5ccc6ncccc6c5)cc4n3)nc(-c3ccccc3-c3ccccc3)n2)cc1 ; c1ccc(-c2ccc(-c3ccc(-c4nc(-c5ccccc5)nc(-c5ccc6ccc(-c7ccc8ncccc8c7)cc6n5)n4)cc3)cc2)cc1]

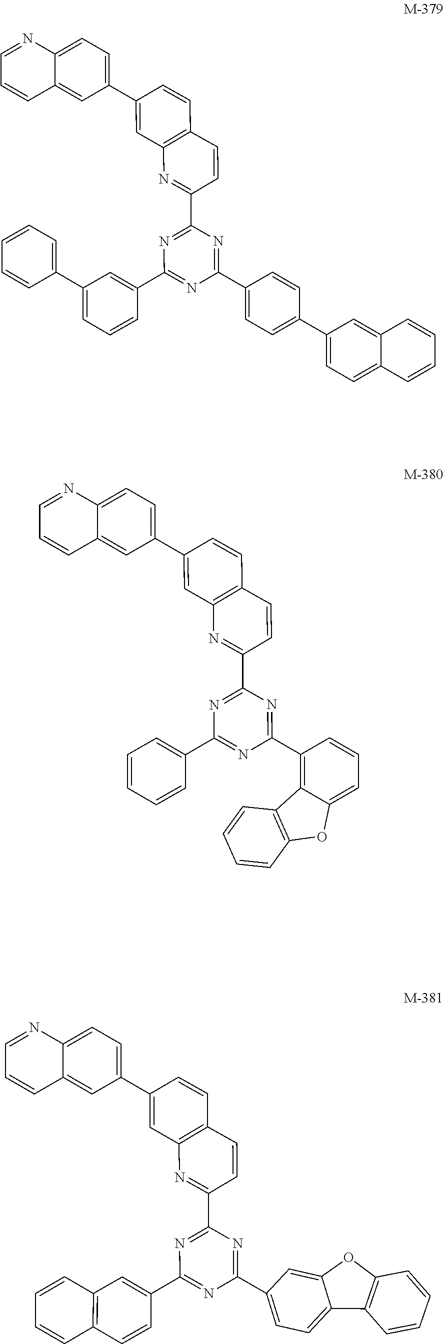 [SMILES: c1ccc(-c2cccc(-c3nc(-c4ccc(-c5ccc6ccccc6c5)cc4)nc(-c4ccc5ccc(-c6ccc7ncccc7c6)cc5n4)n3)c2)cc1 ; c1ccc(-c2nc(-c3ccc4ccc(-c5ccc6ncccc6c5)cc4n3)nc(-c3cccc4oc5ccccc5c34)n2)cc1 ; c1ccc2cc(-c3nc(-c4ccc5c(c4)oc4ccccc45)nc(-c4ccc5ccc(-c6ccc7ncccc7c6)cc5n4)n3)ccc2c1]

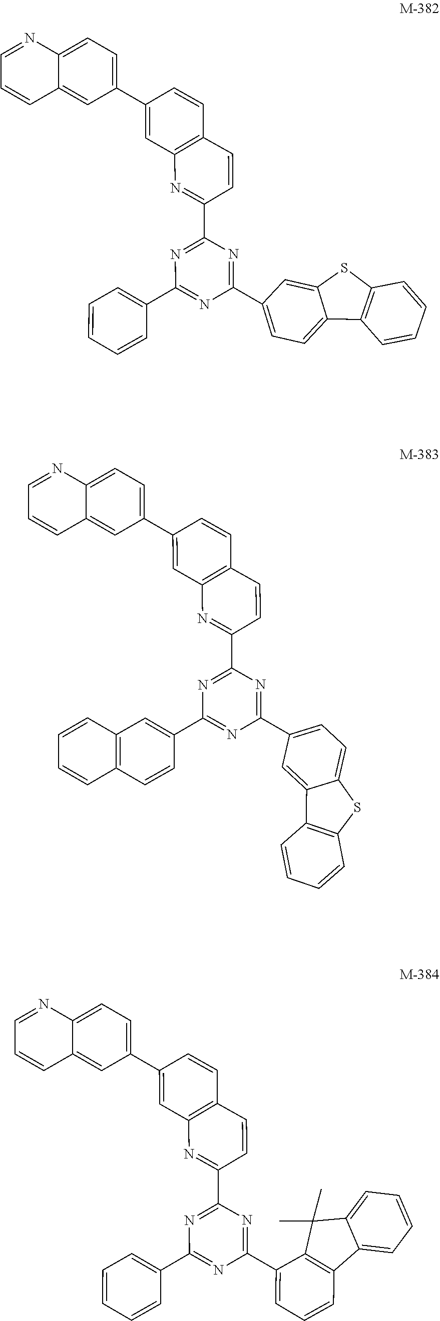 [SMILES: c1ccc(-c2nc(-c3ccc4c(c3)sc3ccccc34)nc(-c3ccc4ccc(-c5ccc6ncccc6c5)cc4n3)n2)cc1 ; c1ccc2cc(-c3nc(-c4ccc5sc6ccccc6c5c4)nc(-c4ccc5ccc(-c6ccc7ncccc7c6)cc5n4)n3)ccc2c1 ; CC1(C)c2ccccc2-c2cccc(-c3nc(-c4ccccc4)nc(-c4ccc5ccc(-c6ccc7ncccc7c6)cc5n4)n3)c21]

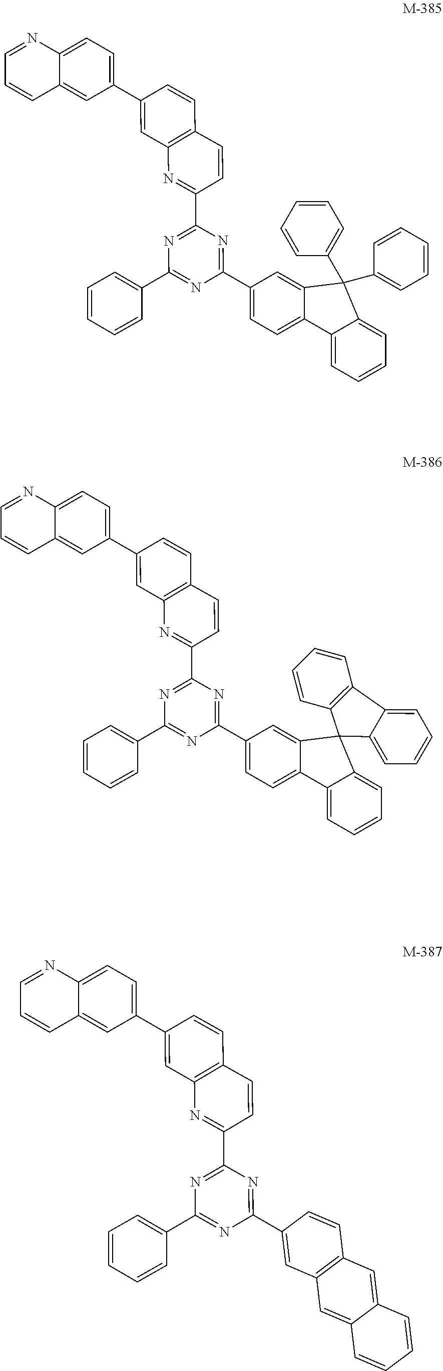 [SMILES: c1ccc(-c2nc(-c3ccc4c(c3)C(c3ccccc3)(c3ccccc3)c3ccccc3-4)nc(-c3ccc4ccc(-c5ccc6ncccc6c5)cc4n3)n2)cc1 ; c1ccc(-c2nc(-c3ccc4c(c3)C3(c5ccccc5-c5ccccc53)c3ccccc3-4)nc(-c3ccc4ccc(-c5ccc6ncccc6c5)cc4n3)n2)cc1 ; c1ccc(-c2nc(-c3ccc4cc5ccccc5cc4c3)nc(-c3ccc4ccc(-c5ccc6ncccc6c5)cc4n3)n2)cc1]

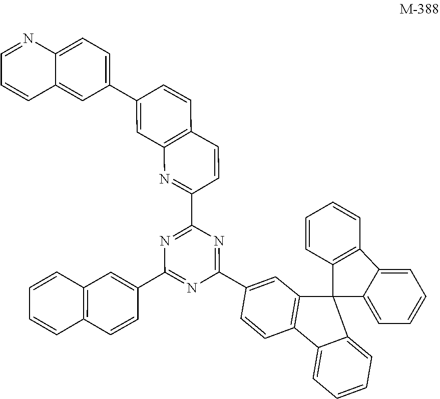 [SMILES: c1ccc2c(c1)-c1ccccc1C21c2ccccc2-c2ccc(-c3nc(-c4ccc5ccccc5c4)nc(-c4ccc5ccc(-c6ccc7ncccc7c6)cc5n4)n3)cc21]

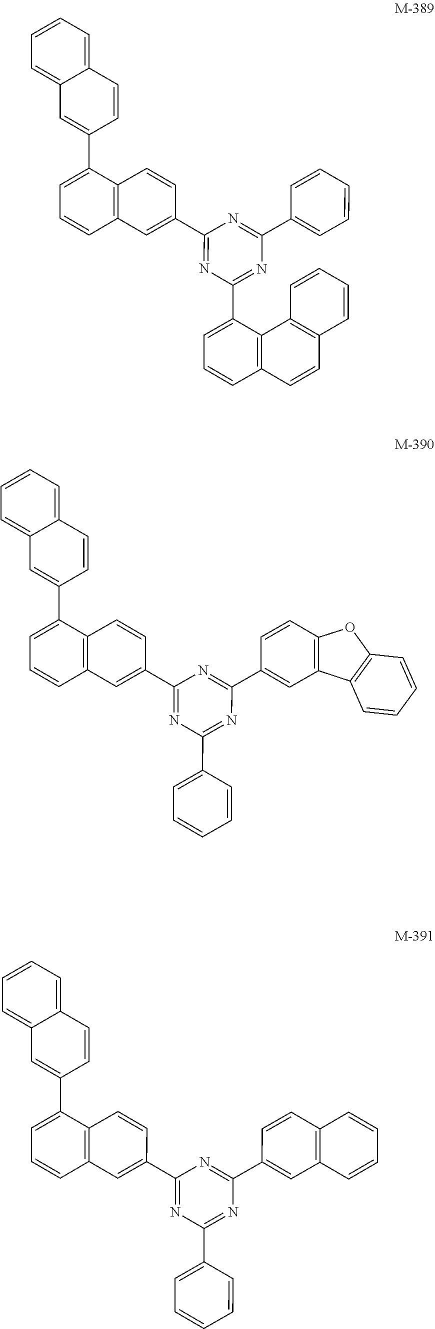 [SMILES: c1ccc(-c2nc(-c3ccc4c(-c5ccc6ccccc6c5)cccc4c3)nc(-c3cccc4ccc5ccccc5c34)n2)cc1 ; c1ccc(-c2nc(-c3ccc4c(-c5ccc6ccccc6c5)cccc4c3)nc(-c3ccc4oc5ccccc5c4c3)n2)cc1 ; c1ccc(-c2nc(-c3ccc4ccccc4c3)nc(-c3ccc4c(-c5ccc6ccccc6c5)cccc4c3)n2)cc1]

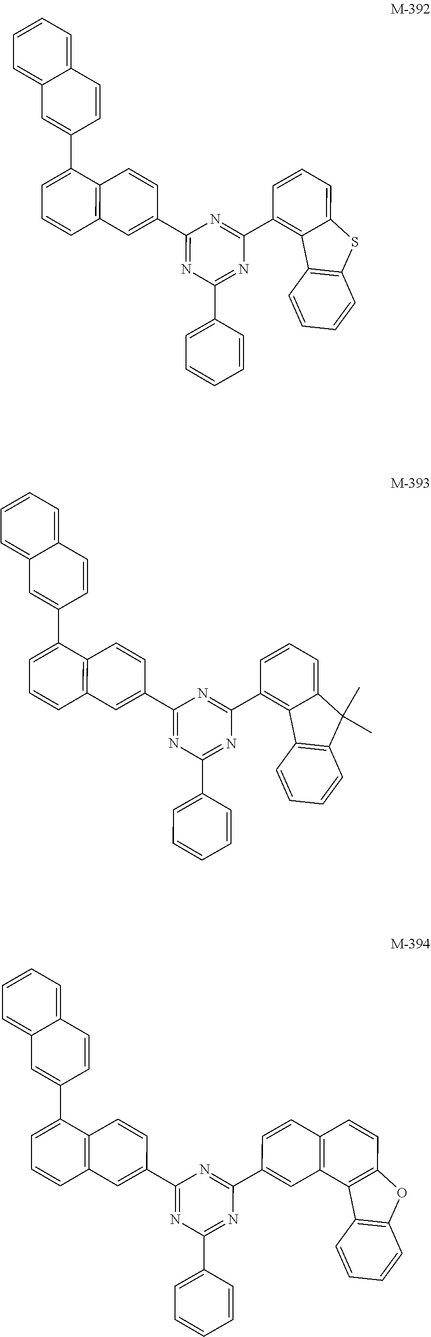 [SMILES: c1ccc(-c2nc(-c3ccc4c(-c5ccc6ccccc6c5)cccc4c3)nc(-c3cccc4sc5ccccc5c34)n2)cc1 ; CC1(C)c2ccccc2-c2c(-c3nc(-c4ccccc4)nc(-c4ccc5c(-c6ccc7ccccc7c6)cccc5c4)n3)cccc21 ; c1ccc(-c2nc(-c3ccc4c(-c5ccc6ccccc6c5)cccc4c3)nc(-c3ccc4ccc5oc6ccccc6c5c4c3)n2)cc1]

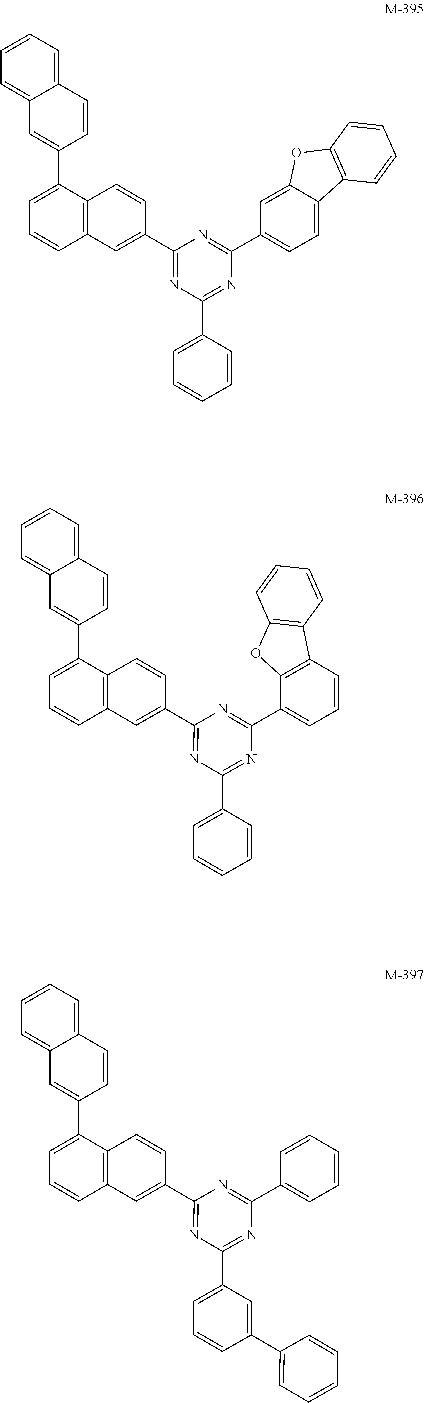 [SMILES: c1ccc(-c2nc(-c3ccc4c(-c5ccc6ccccc6c5)cccc4c3)nc(-c3ccc4c(c3)oc3ccccc34)n2)cc1 ; c1ccc(-c2nc(-c3ccc4c(-c5ccc6ccccc6c5)cccc4c3)nc(-c3cccc4c3oc3ccccc34)n2)cc1 ; c1ccc(-c2cccc(-c3nc(-c4ccccc4)nc(-c4ccc5c(-c6ccc7ccccc7c6)cccc5c4)n3)c2)cc1]

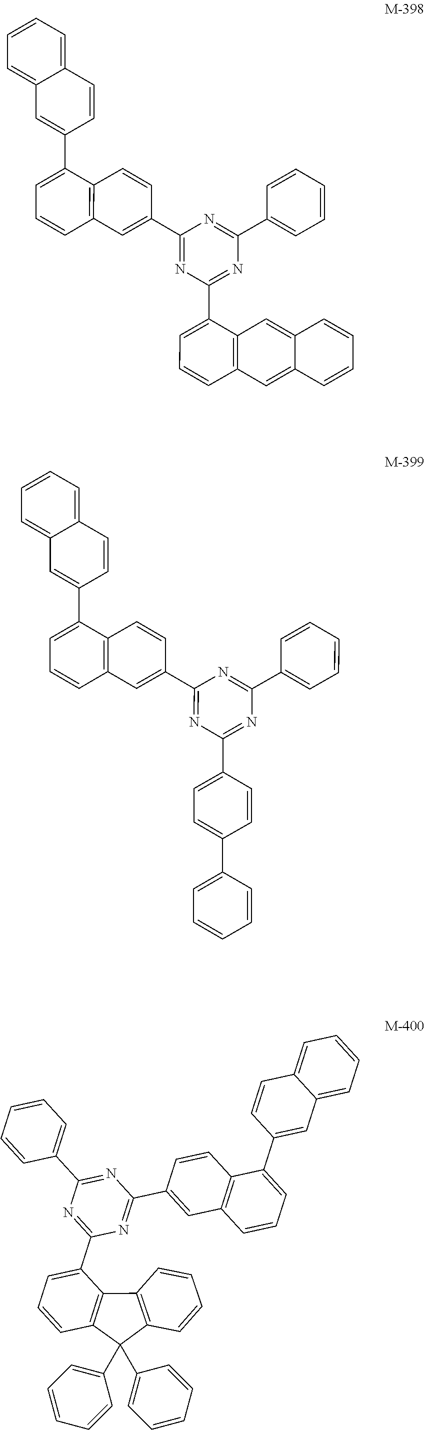 [SMILES: c1ccc(-c2nc(-c3ccc4c(-c5ccc6ccccc6c5)cccc4c3)nc(-c3cccc4cc5ccccc5cc34)n2)cc1 ; c1ccc(-c2ccc(-c3nc(-c4ccccc4)nc(-c4ccc5c(-c6ccc7ccccc7c6)cccc5c4)n3)cc2)cc1 ; c1ccc(-c2nc(-c3ccc4c(-c5ccc6ccccc6c5)cccc4c3)nc(-c3cccc4c3-c3ccccc3C4(c3ccccc3)c3ccccc3)n2)cc1]

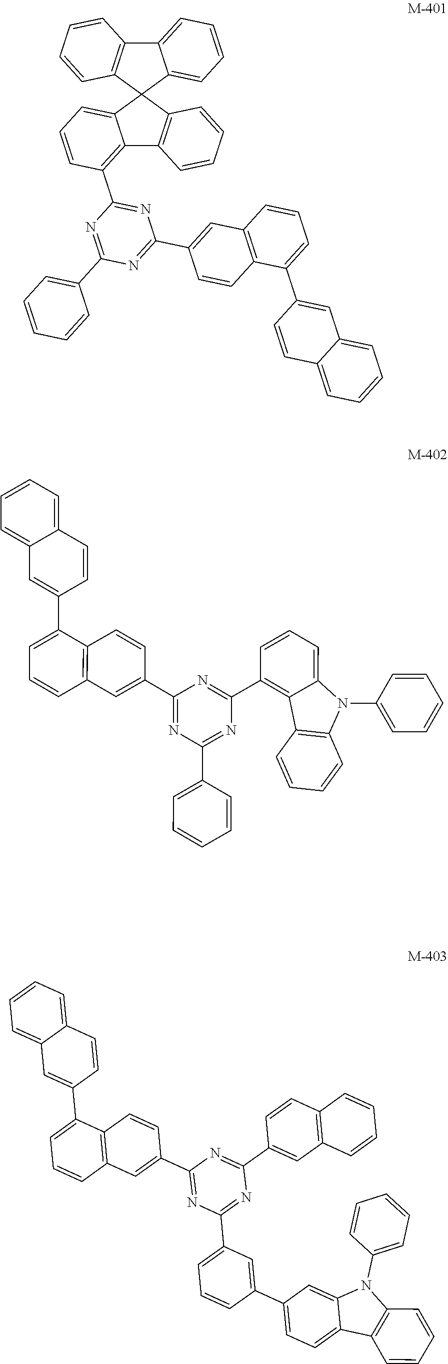 [SMILES: c1ccc(-c2nc(-c3ccc4c(-c5ccc6ccccc6c5)cccc4c3)nc(-c3cccc4c3-c3ccccc3C43c4ccccc4-c4ccccc43)n2)cc1 ; c1ccc(-c2nc(-c3ccc4c(-c5ccc6ccccc6c5)cccc4c3)nc(-c3cccc4c3c3ccccc3n4-c3ccccc3)n2)cc1 ; c1ccc(-n2c3ccccc3c3ccc(-c4cccc(-c5nc(-c6ccc7ccccc7c6)nc(-c6ccc7c(-c8ccc9ccccc9c8)cccc7c6)n5)c4)cc32)cc1]

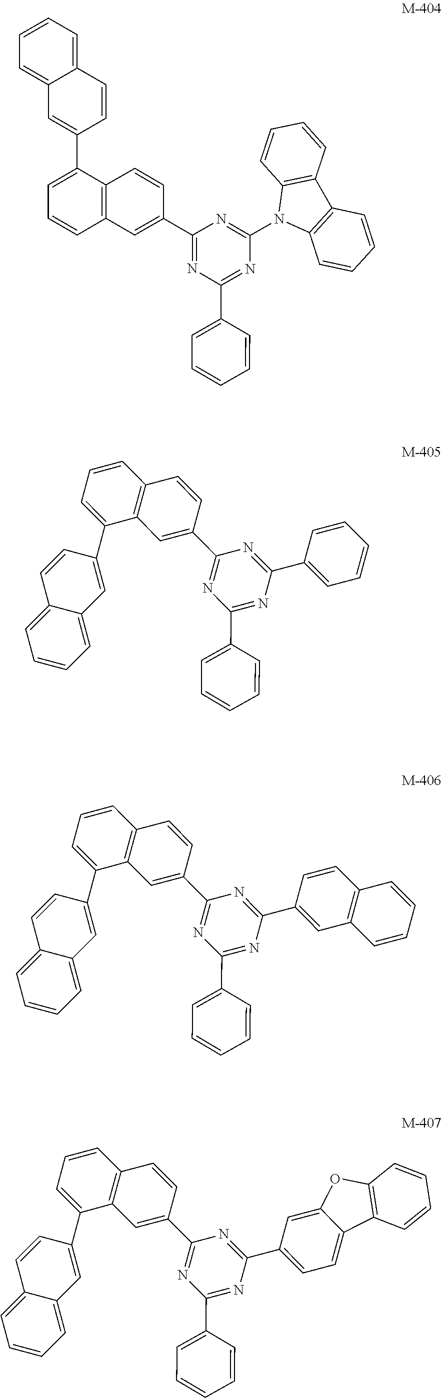 [SMILES: c1ccc(-c2nc(-c3ccc4c(-c5ccc6ccccc6c5)cccc4c3)nc(-n3c4ccccc4c4ccccc43)n2)cc1 ; c1ccc(-c2nc(-c3ccccc3)nc(-c3ccc4cccc(-c5ccc6ccccc6c5)c4c3)n2)cc1 ; c1ccc(-c2nc(-c3ccc4ccccc4c3)nc(-c3ccc4cccc(-c5ccc6ccccc6c5)c4c3)n2)cc1 ; c1ccc(-c2nc(-c3ccc4c(c3)oc3ccccc34)nc(-c3ccc4cccc(-c5ccc6ccccc6c5)c4c3)n2)cc1]

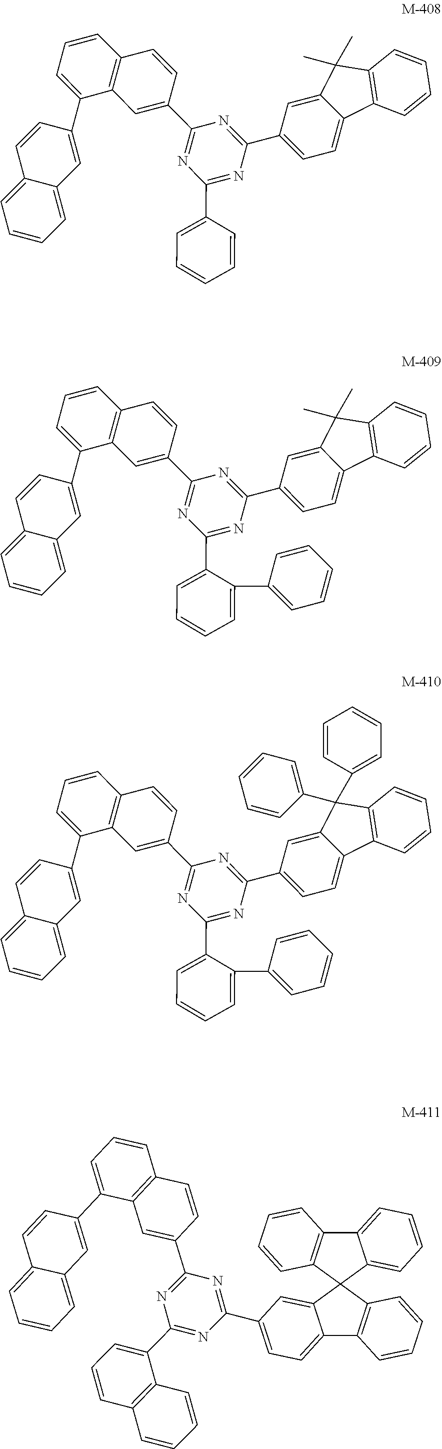 [SMILES: CC1(C)c2ccccc2-c2ccc(-c3nc(-c4ccccc4)nc(-c4ccc5cccc(-c6ccc7ccccc7c6)c5c4)n3)cc21 ; CC1(C)c2ccccc2-c2ccc(-c3nc(-c4ccc5cccc(-c6ccc7ccccc7c6)c5c4)nc(-c4ccccc4-c4ccccc4)n3)cc21 ; c1ccc(-c2ccccc2-c2nc(-c3ccc4c(c3)C(c3ccccc3)(c3ccccc3)c3ccccc3-4)nc(-c3ccc4cccc(-c5ccc6ccccc6c5)c4c3)n2)cc1 ; c1ccc2c(c1)-c1ccccc1C21c2ccccc2-c2ccc(-c3nc(-c4ccc5cccc(-c6ccc7ccccc7c6)c5c4)nc(-c4cccc5ccccc45)n3)cc21]

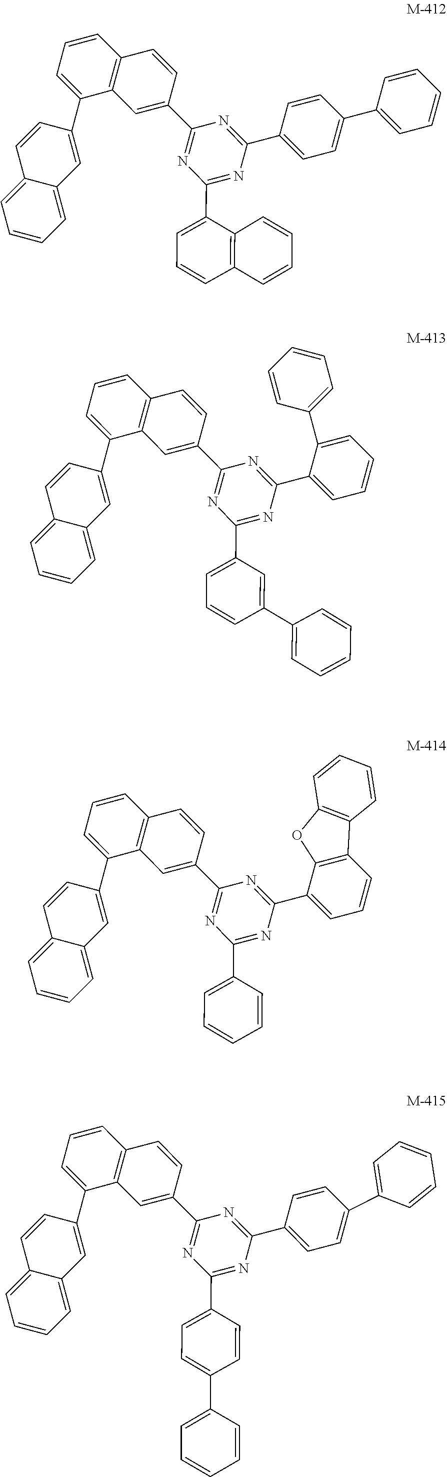 [SMILES: c1ccc(-c2ccc(-c3nc(-c4ccc5cccc(-c6ccc7ccccc7c6)c5c4)nc(-c4cccc5ccccc45)n3)cc2)cc1 ; c1ccc(-c2cccc(-c3nc(-c4ccc5cccc(-c6ccc7ccccc7c6)c5c4)nc(-c4ccccc4-c4ccccc4)n3)c2)cc1 ; c1ccc(-c2nc(-c3ccc4cccc(-c5ccc6ccccc6c5)c4c3)nc(-c3cccc4c3oc3ccccc34)n2)cc1 ; c1ccc(-c2ccc(-c3nc(-c4ccc(-c5ccccc5)cc4)nc(-c4ccc5cccc(-c6ccc7ccccc7c6)c5c4)n3)cc2)cc1]

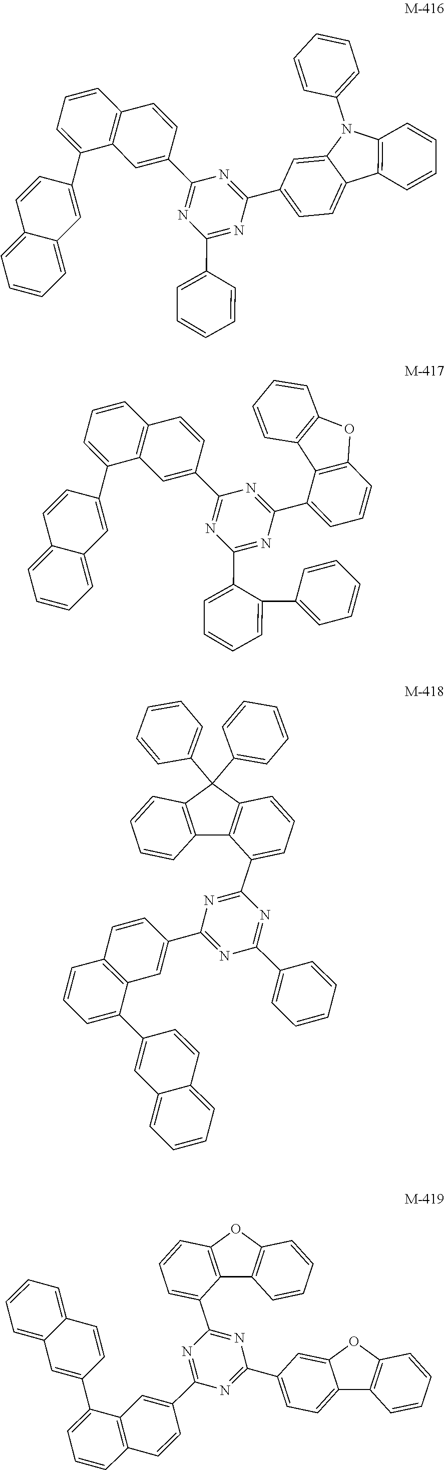 [SMILES: c1ccc(-c2nc(-c3ccc4cccc(-c5ccc6ccccc6c5)c4c3)nc(-c3ccc4c5ccccc5n(-c5ccccc5)c4c3)n2)cc1 ; c1ccc(-c2ccccc2-c2nc(-c3ccc4cccc(-c5ccc6ccccc6c5)c4c3)nc(-c3cccc4oc5ccccc5c34)n2)cc1 ; c1ccc(-c2nc(-c3ccc4cccc(-c5ccc6ccccc6c5)c4c3)nc(-c3cccc4c3-c3ccccc3C4(c3ccccc3)c3ccccc3)n2)cc1 ; c1ccc2cc(-c3cccc4ccc(-c5nc(-c6ccc7c(c6)oc6ccccc67)nc(-c6cccc7oc8ccccc8c67)n5)cc34)ccc2c1]

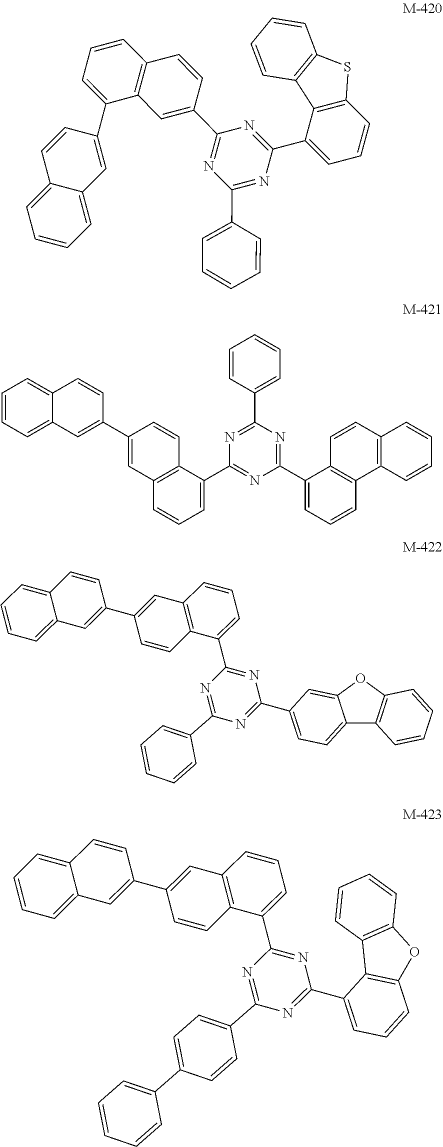 [SMILES: c1ccc(-c2nc(-c3ccc4cccc(-c5ccc6ccccc6c5)c4c3)nc(-c3cccc4sc5ccccc5c34)n2)cc1 ; c1ccc(-c2nc(-c3cccc4cc(-c5ccc6ccccc6c5)ccc34)nc(-c3cccc4c3ccc3ccccc34)n2)cc1 ; c1ccc(-c2nc(-c3ccc4c(c3)oc3ccccc34)nc(-c3cccc4cc(-c5ccc6ccccc6c5)ccc34)n2)cc1 ; c1ccc(-c2ccc(-c3nc(-c4cccc5cc(-c6ccc7ccccc7c6)ccc45)nc(-c4cccc5oc6ccccc6c45)n3)cc2)cc1]

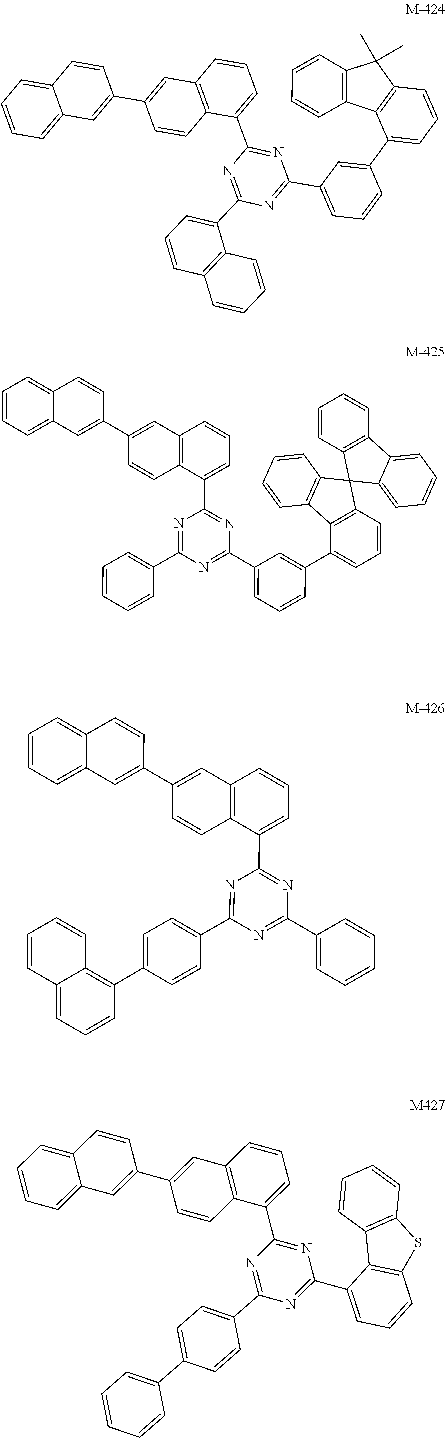 [SMILES: CC1(C)c2ccccc2-c2c(-c3cccc(-c4nc(-c5cccc6ccccc56)nc(-c5cccc6cc(-c7ccc8ccccc8c7)ccc56)n4)c3)cccc21 ; c1ccc(-c2nc(-c3cccc(-c4cccc5c4-c4ccccc4C54c5ccccc5-c5ccccc54)c3)nc(-c3cccc4cc(-c5ccc6ccccc6c5)ccc34)n2)cc1 ; c1ccc(-c2nc(-c3ccc(-c4cccc5ccccc45)cc3)nc(-c3cccc4cc(-c5ccc6ccccc6c5)ccc34)n2)cc1 ; c1ccc(-c2ccc(-c3nc(-c4cccc5cc(-c6ccc7ccccc7c6)ccc45)nc(-c4cccc5sc6ccccc6c45)n3)cc2)cc1]

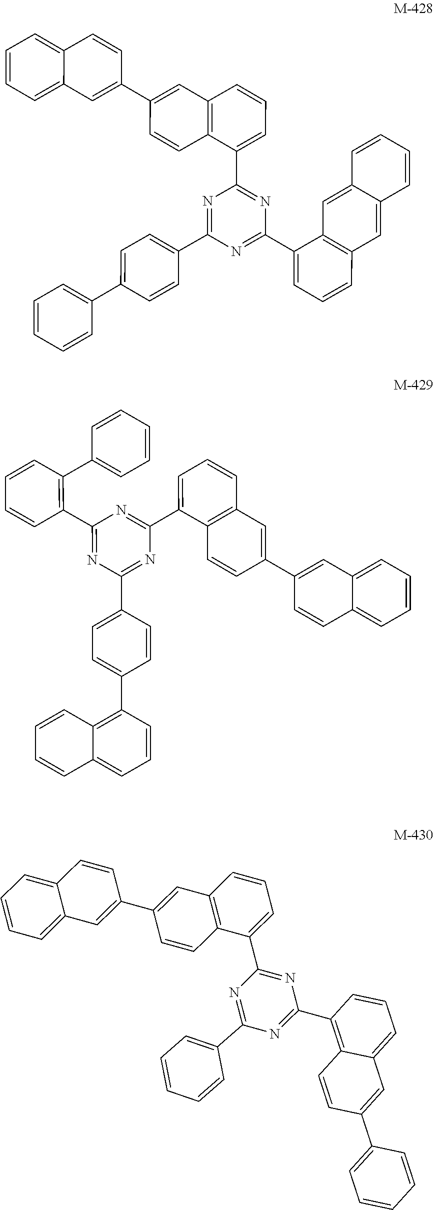 [SMILES: c1ccc(-c2ccc(-c3nc(-c4cccc5cc(-c6ccc7ccccc7c6)ccc45)nc(-c4cccc5cc6ccccc6cc45)n3)cc2)cc1 ; c1ccc(-c2ccccc2-c2nc(-c3ccc(-c4cccc5ccccc45)cc3)nc(-c3cccc4cc(-c5ccc6ccccc6c5)ccc34)n2)cc1 ; c1ccc(-c2ccc3c(-c4nc(-c5ccccc5)nc(-c5cccc6cc(-c7ccc8ccccc8c7)ccc56)n4)cccc3c2)cc1]

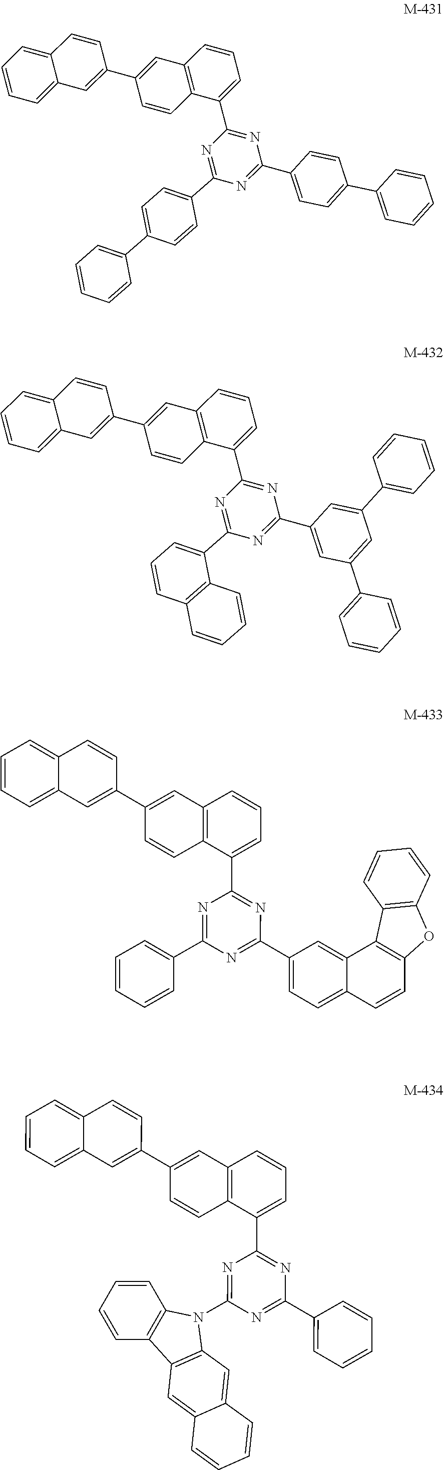 [SMILES: c1ccc(-c2ccc(-c3nc(-c4ccc(-c5ccccc5)cc4)nc(-c4cccc5cc(-c6ccc7ccccc7c6)ccc45)n3)cc2)cc1 ; c1ccc(-c2cc(-c3ccccc3)cc(-c3nc(-c4cccc5ccccc45)nc(-c4cccc5cc(-c6ccc7ccccc7c6)ccc45)n3)c2)cc1 ; c1ccc(-c2nc(-c3ccc4ccc5oc6ccccc6c5c4c3)nc(-c3cccc4cc(-c5ccc6ccccc6c5)ccc34)n2)cc1 ; c1ccc(-c2nc(-c3cccc4cc(-c5ccc6ccccc6c5)ccc34)nc(-n3c4ccccc4c4cc5ccccc5cc43)n2)cc1]

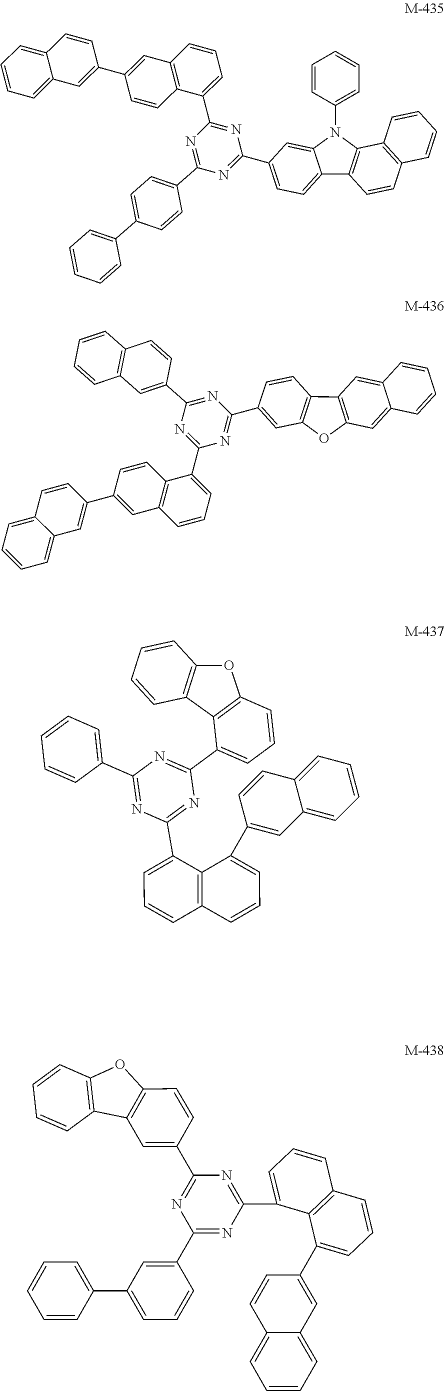 [SMILES: c1ccc(-c2ccc(-c3nc(-c4ccc5c6ccc7ccccc7c6n(-c6ccccc6)c5c4)nc(-c4cccc5cc(-c6ccc7ccccc7c6)ccc45)n3)cc2)cc1 ; c1ccc2cc(-c3ccc4c(-c5nc(-c6ccc7ccccc7c6)nc(-c6ccc7c(c6)oc6cc8ccccc8cc67)n5)cccc4c3)ccc2c1 ; c1ccc(-c2nc(-c3cccc4cccc(-c5ccc6ccccc6c5)c34)nc(-c3cccc4oc5ccccc5c34)n2)cc1 ; c1ccc(-c2cccc(-c3nc(-c4ccc5oc6ccccc6c5c4)nc(-c4cccc5cccc(-c6ccc7ccccc7c6)c45)n3)c2)cc1]

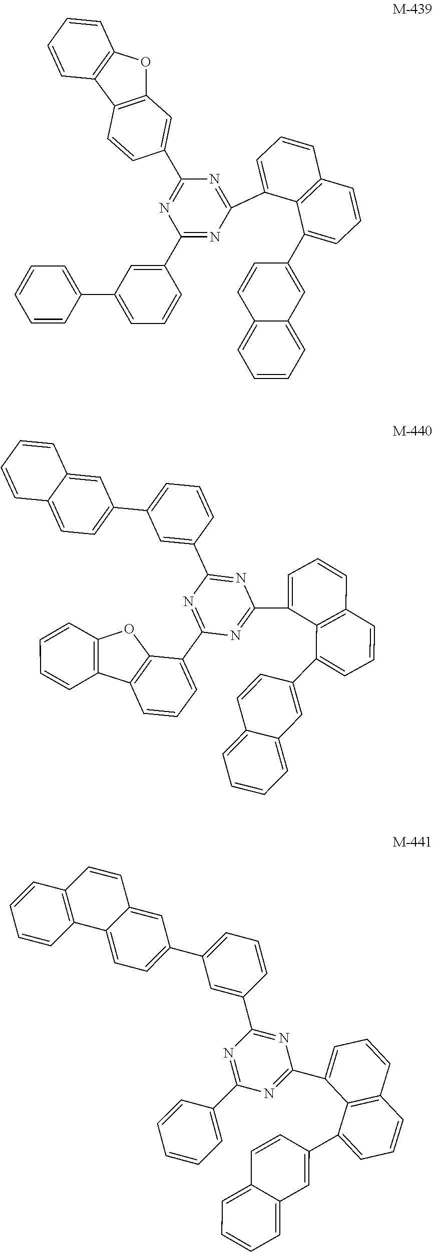 [SMILES: c1ccc(-c2cccc(-c3nc(-c4ccc5c(c4)oc4ccccc45)nc(-c4cccc5cccc(-c6ccc7ccccc7c6)c45)n3)c2)cc1 ; c1cc(-c2ccc3ccccc3c2)cc(-c2nc(-c3cccc4c3oc3ccccc34)nc(-c3cccc4cccc(-c5ccc6ccccc6c5)c34)n2)c1 ; c1ccc(-c2nc(-c3cccc(-c4ccc5c(ccc6ccccc65)c4)c3)nc(-c3cccc4cccc(-c5ccc6ccccc6c5)c34)n2)cc1]

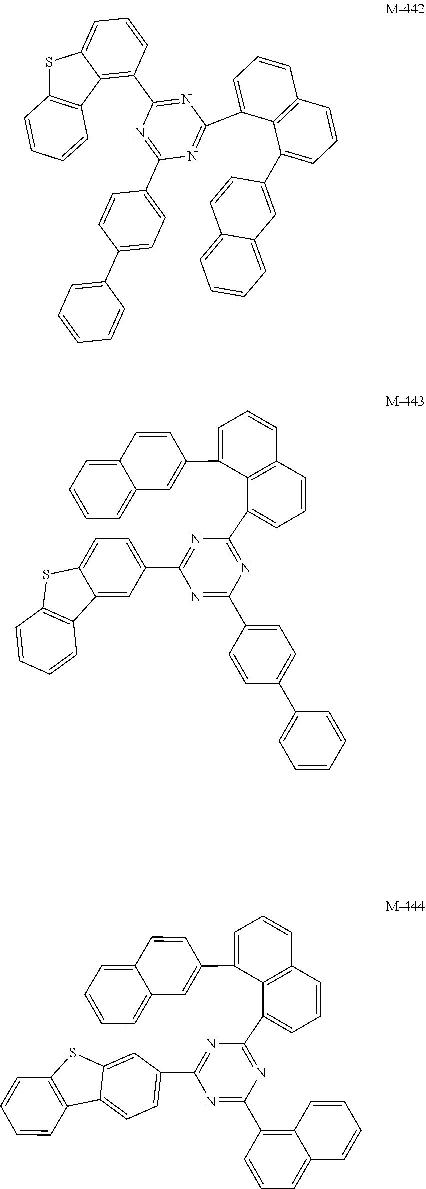 [SMILES: c1ccc(-c2ccc(-c3nc(-c4cccc5cccc(-c6ccc7ccccc7c6)c45)nc(-c4cccc5sc6ccccc6c45)n3)cc2)cc1 ; c1ccc(-c2ccc(-c3nc(-c4ccc5sc6ccccc6c5c4)nc(-c4cccc5cccc(-c6ccc7ccccc7c6)c45)n3)cc2)cc1 ; c1ccc2cc(-c3cccc4cccc(-c5nc(-c6ccc7c(c6)sc6ccccc67)nc(-c6cccc7ccccc67)n5)c34)ccc2c1]

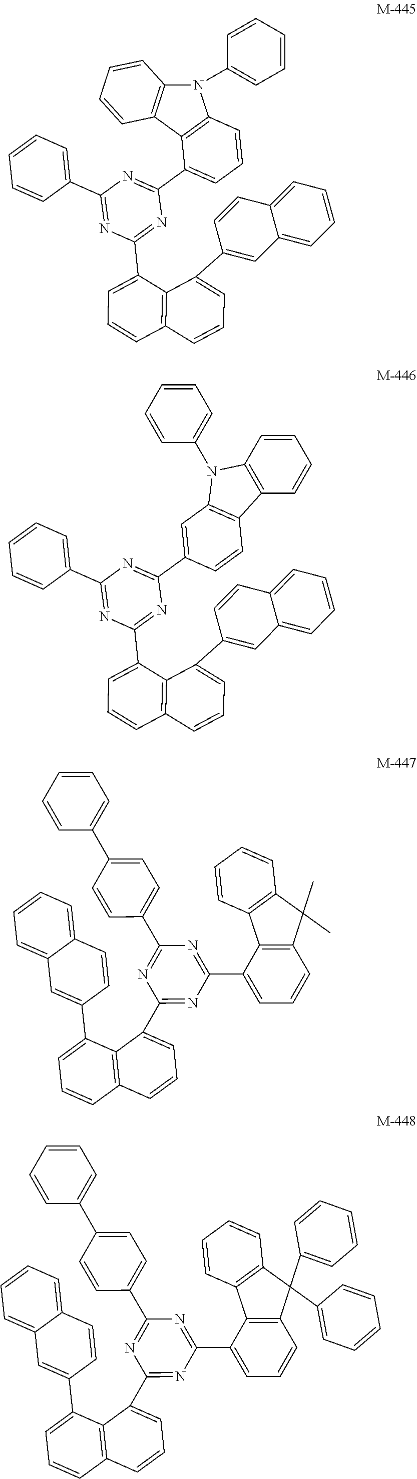

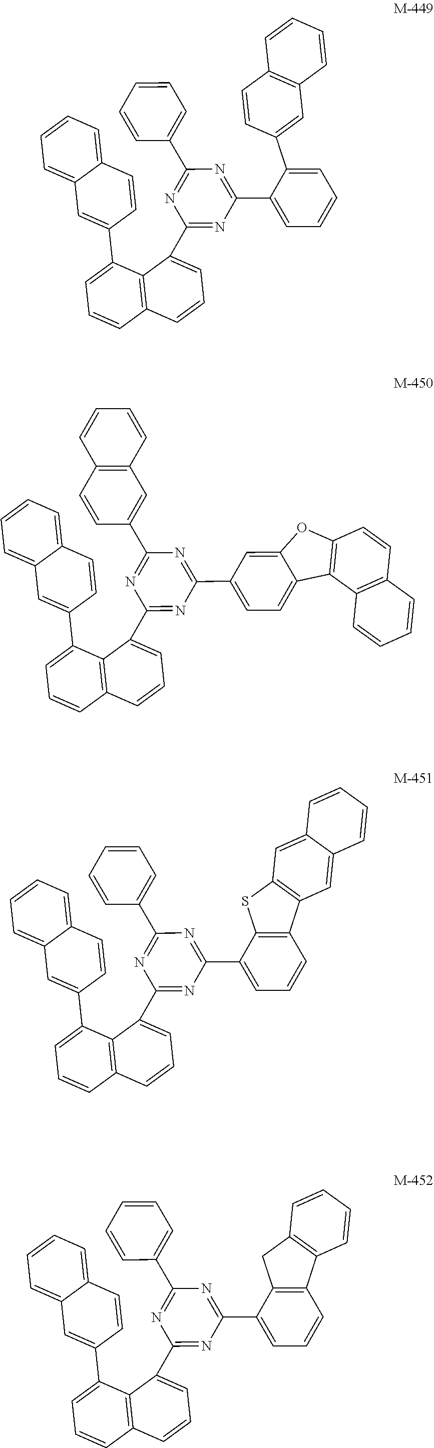 [SMILES: c1ccc(-c2nc(-c3ccccc3-c3ccc4ccccc4c3)nc(-c3cccc4cccc(-c5ccc6ccccc6c5)c34)n2)cc1 ; c1ccc2cc(-c3nc(-c4ccc5c(c4)oc4ccc6ccccc6c45)nc(-c4cccc5cccc(-c6ccc7ccccc7c6)c45)n3)ccc2c1 ; c1ccc(-c2nc(-c3cccc4c3sc3cc5ccccc5cc34)nc(-c3cccc4cccc(-c5ccc6ccccc6c5)c34)n2)cc1 ; c1ccc(-c2nc(-c3cccc4c3Cc3ccccc3-4)nc(-c3cccc4cccc(-c5ccc6ccccc6c5)c34)n2)cc1]

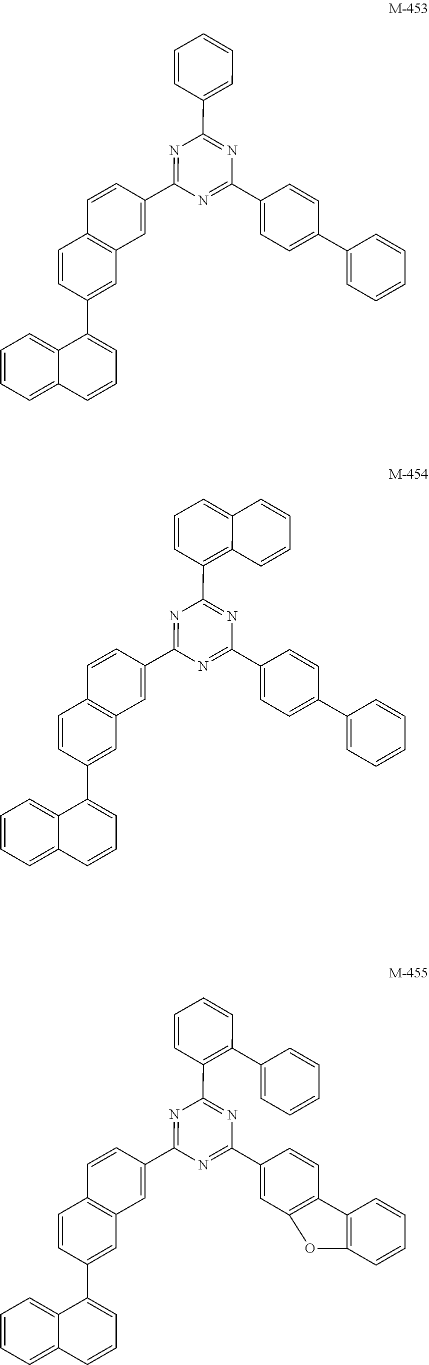 [SMILES: c1ccc(-c2ccc(-c3nc(-c4ccccc4)nc(-c4ccc5ccc(-c6cccc7ccccc67)cc5c4)n3)cc2)cc1 ; c1ccc(-c2ccc(-c3nc(-c4ccc5ccc(-c6cccc7ccccc67)cc5c4)nc(-c4cccc5ccccc45)n3)cc2)cc1 ; c1ccc(-c2ccccc2-c2nc(-c3ccc4ccc(-c5cccc6ccccc56)cc4c3)nc(-c3ccc4c(c3)oc3ccccc34)n2)cc1]

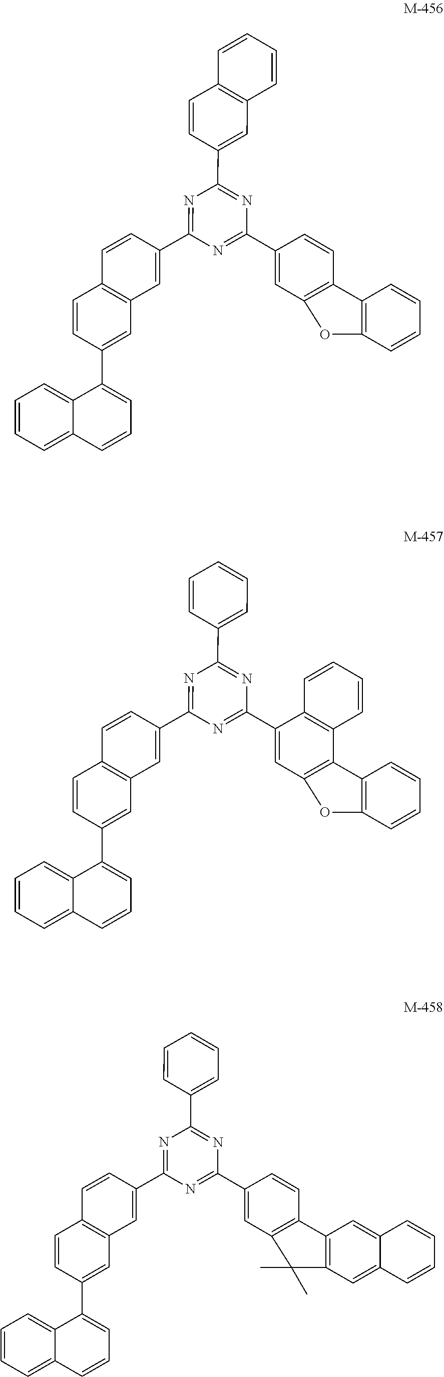 [SMILES: c1ccc2cc(-c3nc(-c4ccc5ccc(-c6cccc7ccccc67)cc5c4)nc(-c4ccc5c(c4)oc4ccccc45)n3)ccc2c1 ; c1ccc(-c2nc(-c3ccc4ccc(-c5cccc6ccccc56)cc4c3)nc(-c3cc4oc5ccccc5c4c4ccccc34)n2)cc1 ; CC1(C)c2cc(-c3nc(-c4ccccc4)nc(-c4ccc5ccc(-c6cccc7ccccc67)cc5c4)n3)ccc2-c2cc3ccccc3cc21]

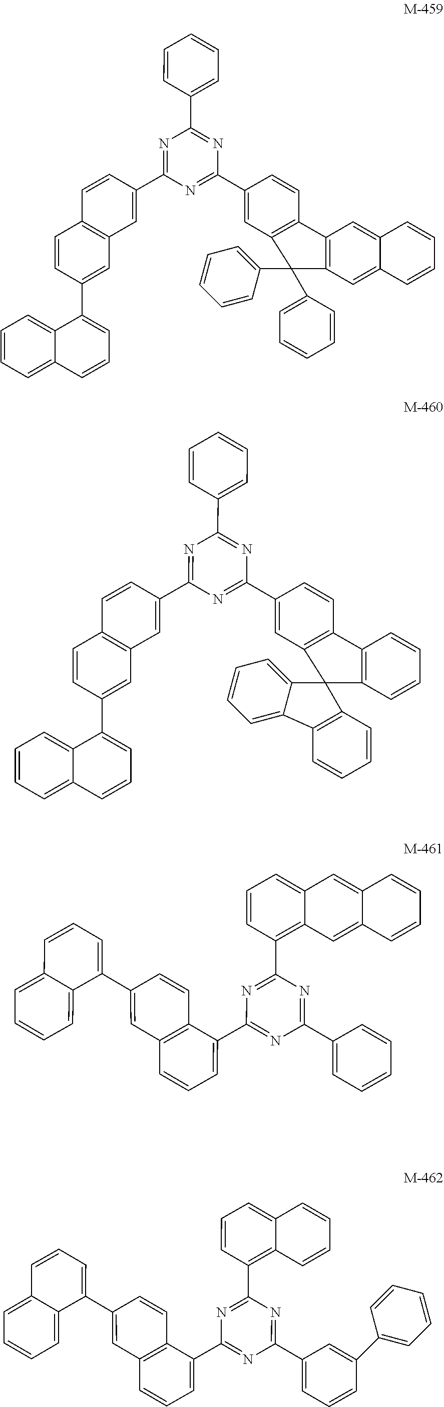 [SMILES: c1ccc(-c2nc(-c3ccc4c(c3)C(c3ccccc3)(c3ccccc3)c3cc5ccccc5cc3-4)nc(-c3ccc4ccc(-c5cccc6ccccc56)cc4c3)n2)cc1 ; c1ccc(-c2nc(-c3ccc4c(c3)C3(c5ccccc5-c5ccccc53)c3ccccc3-4)nc(-c3ccc4ccc(-c5cccc6ccccc56)cc4c3)n2)cc1 ; c1ccc(-c2nc(-c3cccc4cc(-c5cccc6ccccc56)ccc34)nc(-c3cccc4cc5ccccc5cc34)n2)cc1 ; c1ccc(-c2cccc(-c3nc(-c4cccc5ccccc45)nc(-c4cccc5cc(-c6cccc7ccccc67)ccc45)n3)c2)cc1]

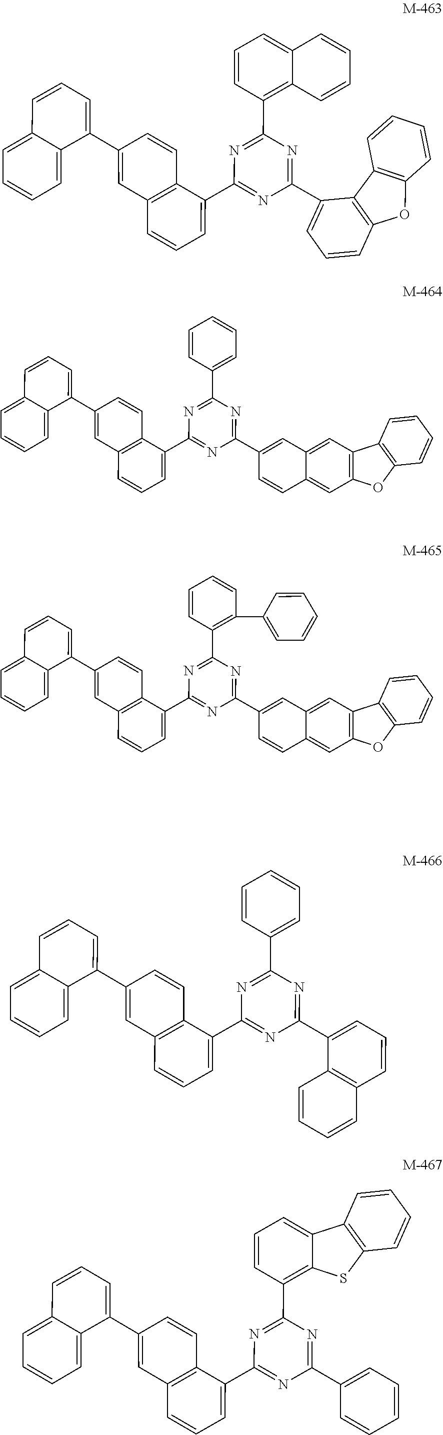 [SMILES: c1ccc2c(-c3ccc4c(-c5nc(-c6cccc7ccccc67)nc(-c6cccc7oc8ccccc8c67)n5)cccc4c3)cccc2c1 ; c1ccc(-c2nc(-c3ccc4cc5oc6ccccc6c5cc4c3)nc(-c3cccc4cc(-c5cccc6ccccc56)ccc34)n2)cc1 ; c1ccc(-c2ccccc2-c2nc(-c3ccc4cc5oc6ccccc6c5cc4c3)nc(-c3cccc4cc(-c5cccc6ccccc56)ccc34)n2)cc1 ; c1ccc(-c2nc(-c3cccc4ccccc34)nc(-c3cccc4cc(-c5cccc6ccccc56)ccc34)n2)cc1 ; c1ccc(-c2nc(-c3cccc4cc(-c5cccc6ccccc56)ccc34)nc(-c3cccc4c3sc3ccccc34)n2)cc1]

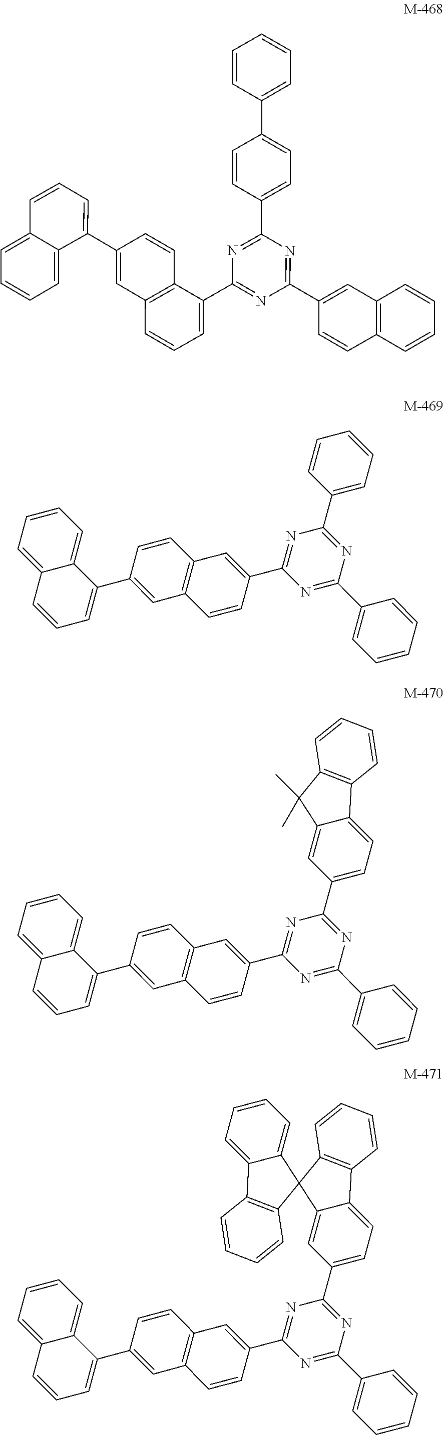 [SMILES: c1ccc(-c2ccc(-c3nc(-c4ccc5ccccc5c4)nc(-c4cccc5cc(-c6cccc7ccccc67)ccc45)n3)cc2)cc1 ; c1ccc(-c2nc(-c3ccccc3)nc(-c3ccc4cc(-c5cccc6ccccc56)ccc4c3)n2)cc1 ; CC1(C)c2ccccc2-c2ccc(-c3nc(-c4ccccc4)nc(-c4ccc5cc(-c6cccc7ccccc67)ccc5c4)n3)cc21 ; c1ccc(-c2nc(-c3ccc4c(c3)C3(c5ccccc5-c5ccccc53)c3ccccc3-4)nc(-c3ccc4cc(-c5cccc6ccccc56)ccc4c3)n2)cc1]

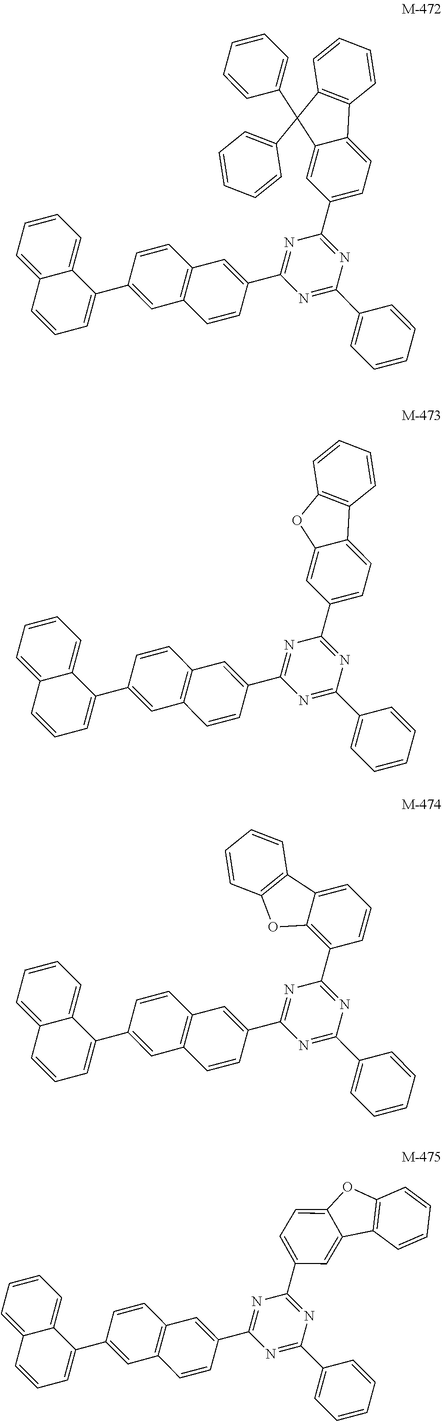 [SMILES: c1ccc(-c2nc(-c3ccc4c(c3)C(c3ccccc3)(c3ccccc3)c3ccccc3-4)nc(-c3ccc4cc(-c5cccc6ccccc56)ccc4c3)n2)cc1 ; c1ccc(-c2nc(-c3ccc4cc(-c5cccc6ccccc56)ccc4c3)nc(-c3ccc4c(c3)oc3ccccc34)n2)cc1 ; c1ccc(-c2nc(-c3ccc4cc(-c5cccc6ccccc56)ccc4c3)nc(-c3cccc4c3oc3ccccc34)n2)cc1 ; c1ccc(-c2nc(-c3ccc4cc(-c5cccc6ccccc56)ccc4c3)nc(-c3ccc4oc5ccccc5c4c3)n2)cc1]

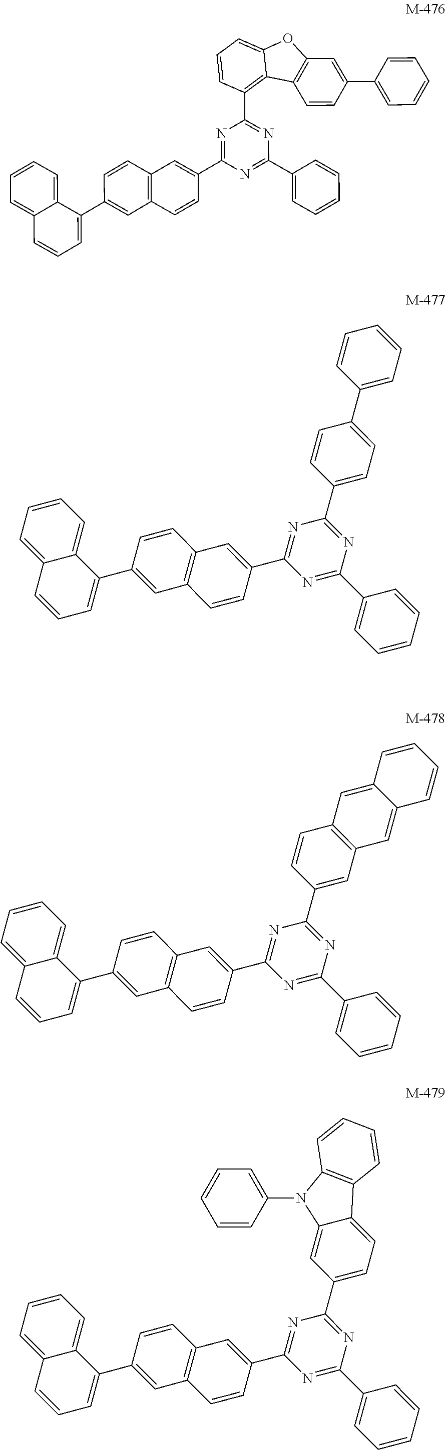 [SMILES: c1ccc(-c2ccc3c(c2)oc2cccc(-c4nc(-c5ccccc5)nc(-c5ccc6cc(-c7cccc8ccccc78)ccc6c5)n4)c23)cc1 ; c1ccc(-c2ccc(-c3nc(-c4ccccc4)nc(-c4ccc5cc(-c6cccc7ccccc67)ccc5c4)n3)cc2)cc1 ; c1ccc(-c2nc(-c3ccc4cc(-c5cccc6ccccc56)ccc4c3)nc(-c3ccc4cc5ccccc5cc4c3)n2)cc1 ; c1ccc(-c2nc(-c3ccc4cc(-c5cccc6ccccc56)ccc4c3)nc(-c3ccc4c5ccccc5n(-c5ccccc5)c4c3)n2)cc1]

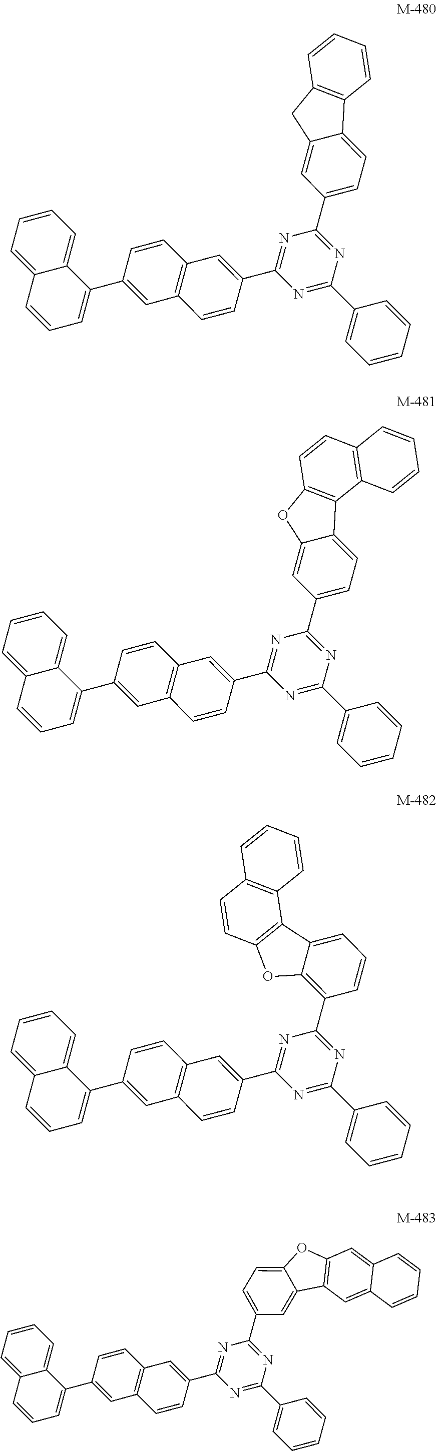 [SMILES: c1ccc(-c2nc(-c3ccc4c(c3)Cc3ccccc3-4)nc(-c3ccc4cc(-c5cccc6ccccc56)ccc4c3)n2)cc1 ; c1ccc(-c2nc(-c3ccc4cc(-c5cccc6ccccc56)ccc4c3)nc(-c3ccc4c(c3)oc3ccc5ccccc5c34)n2)cc1 ; c1ccc(-c2nc(-c3ccc4cc(-c5cccc6ccccc56)ccc4c3)nc(-c3cccc4c3oc3ccc5ccccc5c34)n2)cc1 ; c1ccc(-c2nc(-c3ccc4cc(-c5cccc6ccccc56)ccc4c3)nc(-c3ccc4oc5cc6ccccc6cc5c4c3)n2)cc1]

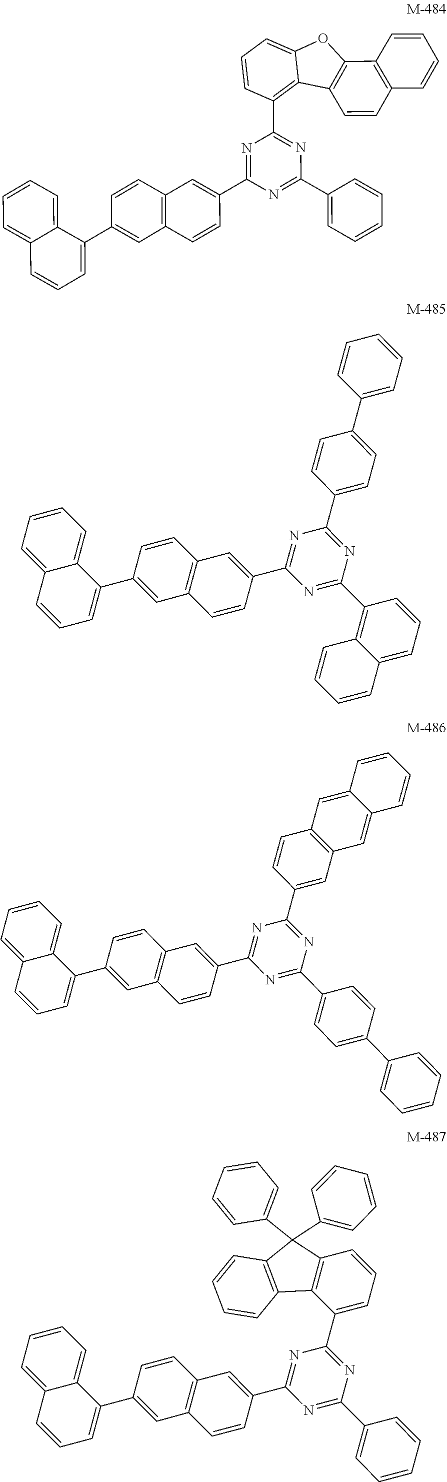 [SMILES: c1ccc(-c2nc(-c3ccc4cc(-c5cccc6ccccc56)ccc4c3)nc(-c3cccc4oc5c6ccccc6ccc5c34)n2)cc1 ; c1ccc(-c2ccc(-c3nc(-c4ccc5cc(-c6cccc7ccccc67)ccc5c4)nc(-c4cccc5ccccc45)n3)cc2)cc1 ; c1ccc(-c2ccc(-c3nc(-c4ccc5cc(-c6cccc7ccccc67)ccc5c4)nc(-c4ccc5cc6ccccc6cc5c4)n3)cc2)cc1 ; c1ccc(-c2nc(-c3ccc4cc(-c5cccc6ccccc56)ccc4c3)nc(-c3cccc4c3-c3ccccc3C4(c3ccccc3)c3ccccc3)n2)cc1]

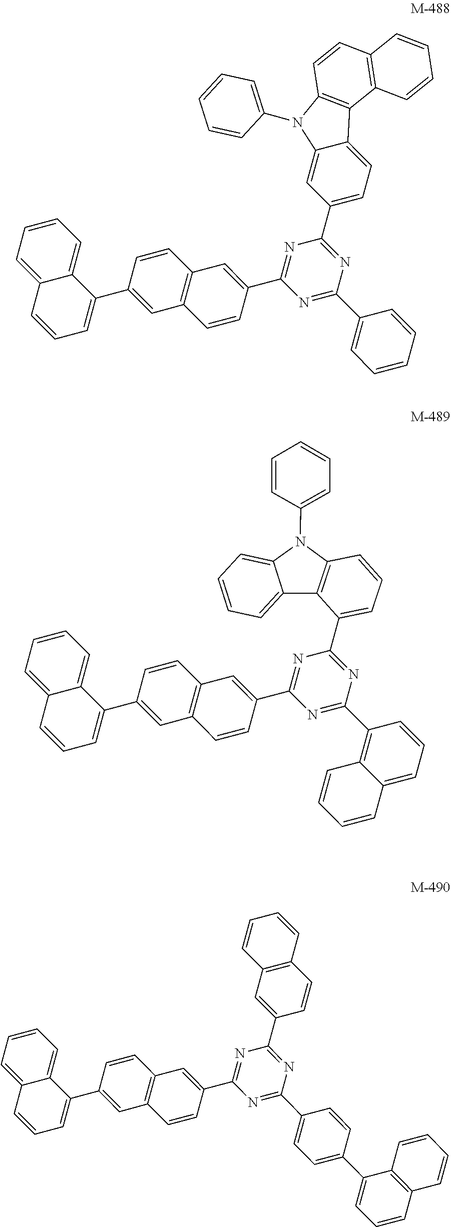 [SMILES: c1ccc(-c2nc(-c3ccc4cc(-c5cccc6ccccc56)ccc4c3)nc(-c3ccc4c5c6ccccc6ccc5n(-c5ccccc5)c4c3)n2)cc1 ; c1ccc(-n2c3ccccc3c3c(-c4nc(-c5ccc6cc(-c7cccc8ccccc78)ccc6c5)nc(-c5cccc6ccccc56)n4)cccc32)cc1 ; c1ccc2cc(-c3nc(-c4ccc(-c5cccc6ccccc56)cc4)nc(-c4ccc5cc(-c6cccc7ccccc67)ccc5c4)n3)ccc2c1]

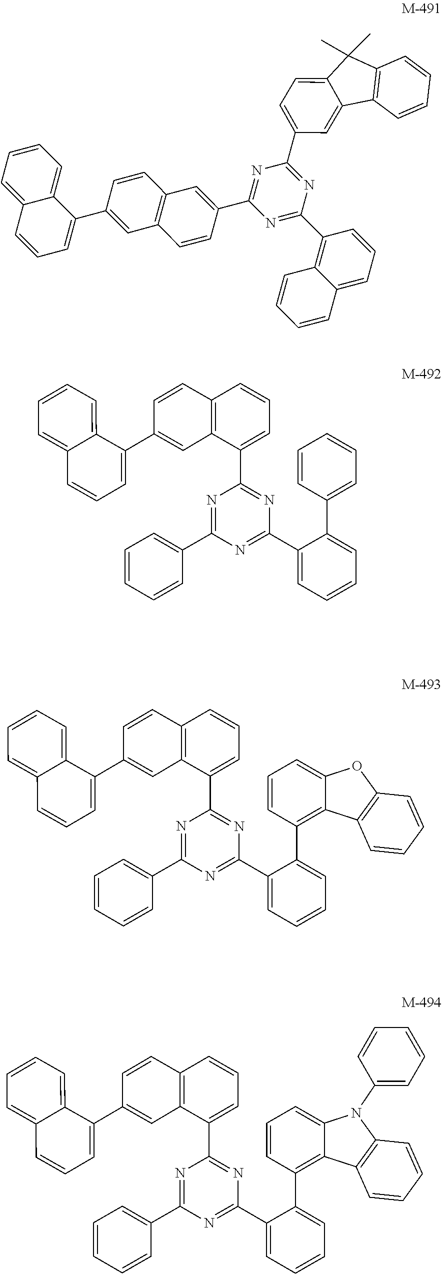 [SMILES: CC1(C)c2ccccc2-c2cc(-c3nc(-c4ccc5cc(-c6cccc7ccccc67)ccc5c4)nc(-c4cccc5ccccc45)n3)ccc21 ; c1ccc(-c2nc(-c3ccccc3-c3ccccc3)nc(-c3cccc4ccc(-c5cccc6ccccc56)cc34)n2)cc1 ; c1ccc(-c2nc(-c3ccccc3-c3cccc4oc5ccccc5c34)nc(-c3cccc4ccc(-c5cccc6ccccc56)cc34)n2)cc1 ; c1ccc(-c2nc(-c3ccccc3-c3cccc4c3c3ccccc3n4-c3ccccc3)nc(-c3cccc4ccc(-c5cccc6ccccc56)cc34)n2)cc1]

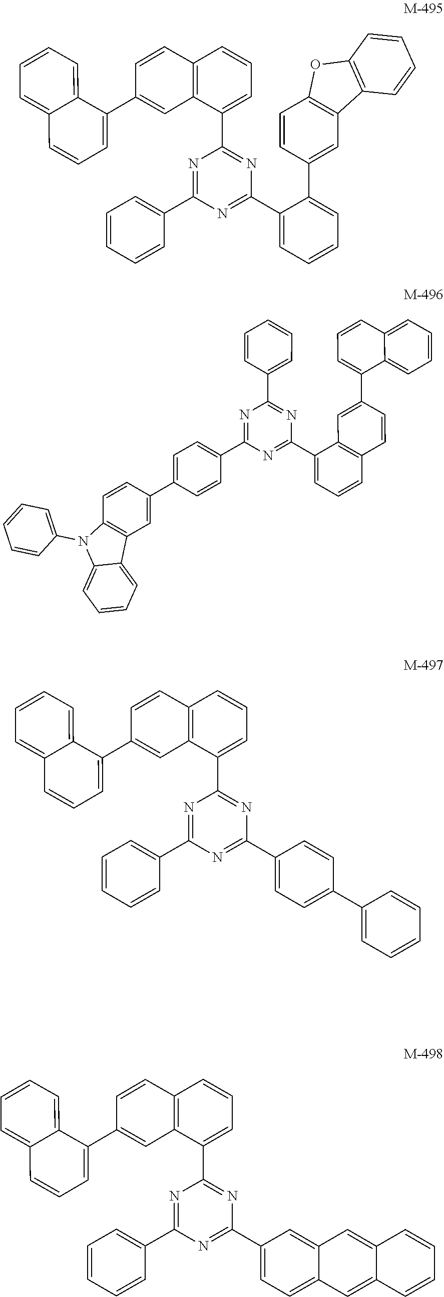 [SMILES: c1ccc(-c2nc(-c3ccccc3-c3ccc4oc5ccccc5c4c3)nc(-c3cccc4ccc(-c5cccc6ccccc56)cc34)n2)cc1 ; c1ccc(-c2nc(-c3ccc(-c4ccc5c(c4)c4ccccc4n5-c4ccccc4)cc3)nc(-c3cccc4ccc(-c5cccc6ccccc56)cc34)n2)cc1 ; c1ccc(-c2ccc(-c3nc(-c4ccccc4)nc(-c4cccc5ccc(-c6cccc7ccccc67)cc45)n3)cc2)cc1 ; c1ccc(-c2nc(-c3ccc4cc5ccccc5cc4c3)nc(-c3cccc4ccc(-c5cccc6ccccc56)cc34)n2)cc1]

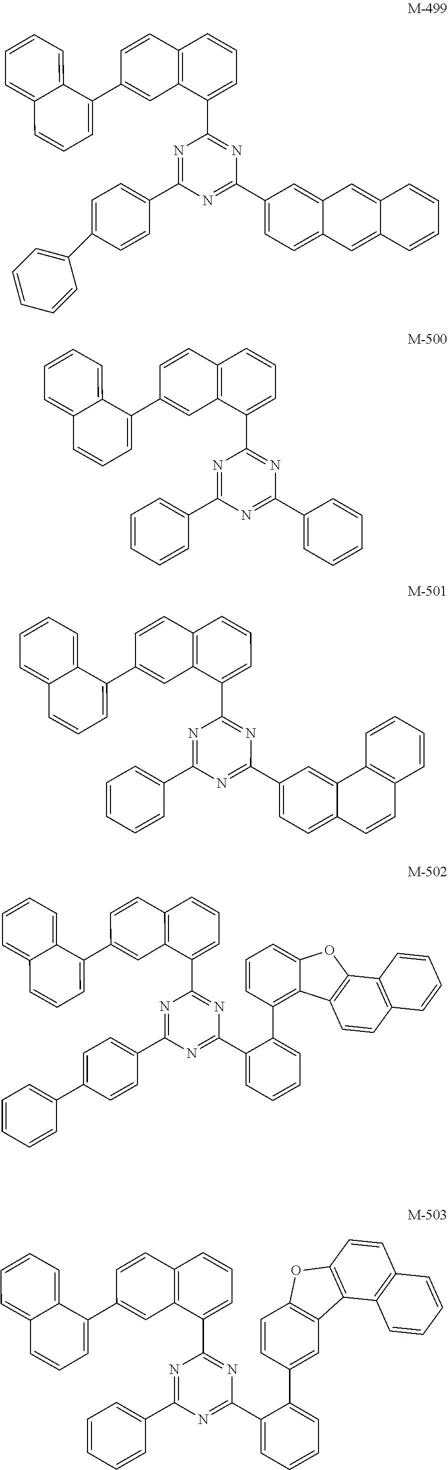

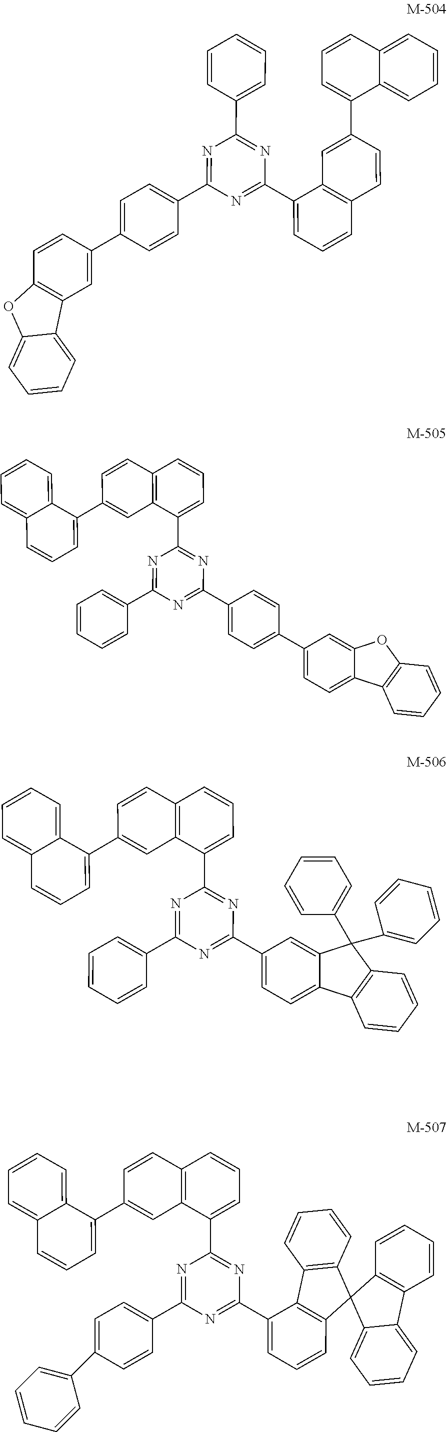 [SMILES: c1ccc(-c2nc(-c3ccc(-c4ccc5oc6ccccc6c5c4)cc3)nc(-c3cccc4ccc(-c5cccc6ccccc56)cc34)n2)cc1 ; c1ccc(-c2nc(-c3ccc(-c4ccc5c(c4)oc4ccccc45)cc3)nc(-c3cccc4ccc(-c5cccc6ccccc56)cc34)n2)cc1 ; c1ccc(-c2nc(-c3ccc4c(c3)C(c3ccccc3)(c3ccccc3)c3ccccc3-4)nc(-c3cccc4ccc(-c5cccc6ccccc56)cc34)n2)cc1 ; c1ccc(-c2ccc(-c3nc(-c4cccc5c4-c4ccccc4C54c5ccccc5-c5ccccc54)nc(-c4cccc5ccc(-c6cccc7ccccc67)cc45)n3)cc2)cc1]

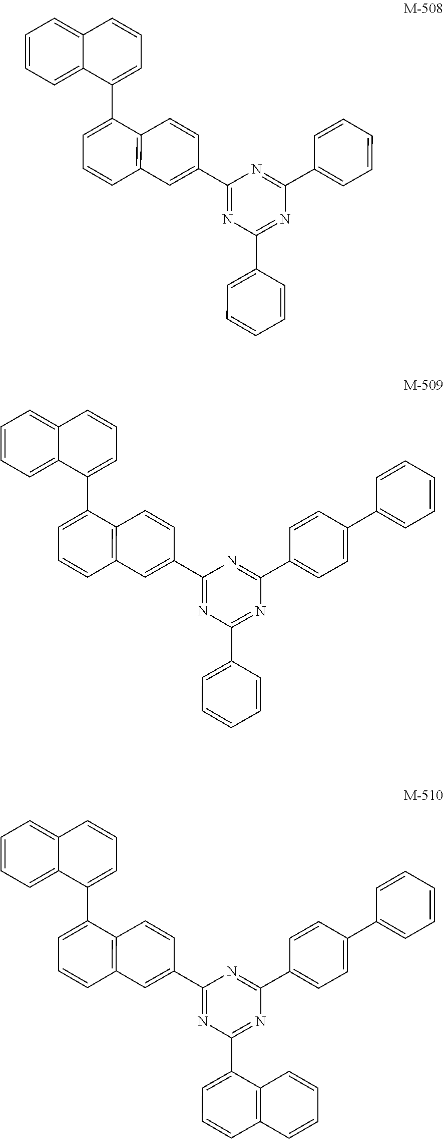 [SMILES: c1ccc(-c2nc(-c3ccccc3)nc(-c3ccc4c(-c5cccc6ccccc56)cccc4c3)n2)cc1 ; c1ccc(-c2ccc(-c3nc(-c4ccccc4)nc(-c4ccc5c(-c6cccc7ccccc67)cccc5c4)n3)cc2)cc1 ; c1ccc(-c2ccc(-c3nc(-c4ccc5c(-c6cccc7ccccc67)cccc5c4)nc(-c4cccc5ccccc45)n3)cc2)cc1]

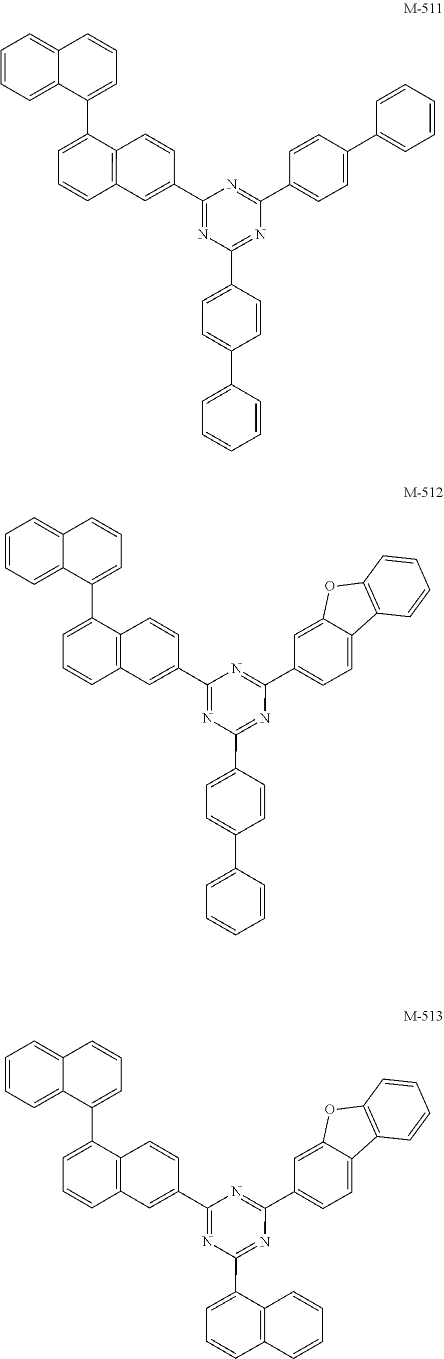 [SMILES: c1ccc(-c2ccc(-c3nc(-c4ccc(-c5ccccc5)cc4)nc(-c4ccc5c(-c6cccc7ccccc67)cccc5c4)n3)cc2)cc1 ; c1ccc(-c2ccc(-c3nc(-c4ccc5c(-c6cccc7ccccc67)cccc5c4)nc(-c4ccc5c(c4)oc4ccccc45)n3)cc2)cc1 ; c1ccc2c(-c3nc(-c4ccc5c(-c6cccc7ccccc67)cccc5c4)nc(-c4ccc5c(c4)oc4ccccc45)n3)cccc2c1]

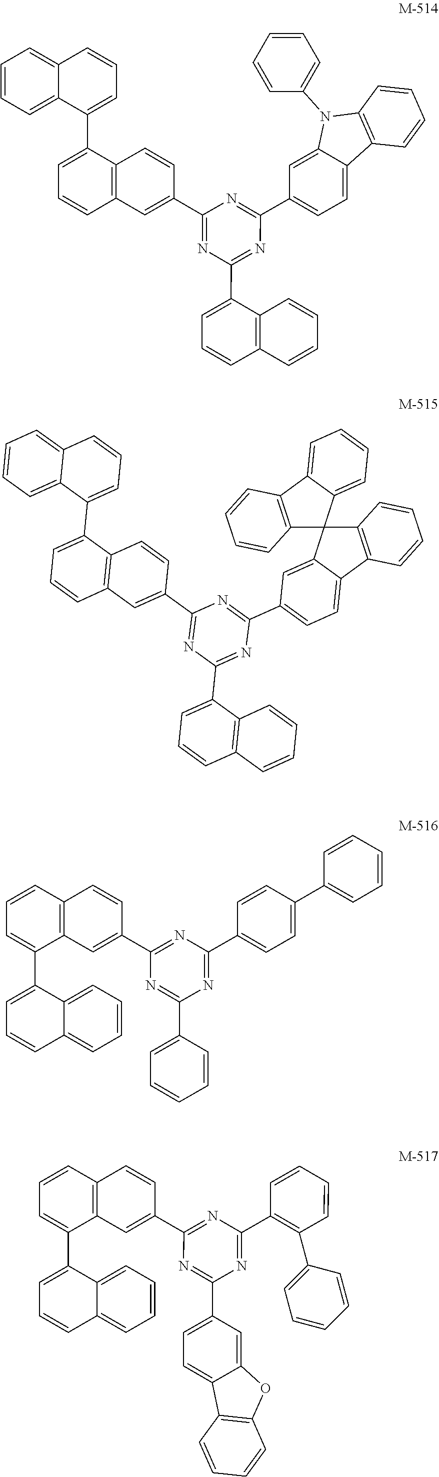 [SMILES: c1ccc(-n2c3ccccc3c3ccc(-c4nc(-c5ccc6c(-c7cccc8ccccc78)cccc6c5)nc(-c5cccc6ccccc56)n4)cc32)cc1 ; c1ccc2c(c1)-c1ccccc1C21c2ccccc2-c2ccc(-c3nc(-c4ccc5c(-c6cccc7ccccc67)cccc5c4)nc(-c4cccc5ccccc45)n3)cc21 ; c1ccc(-c2ccc(-c3nc(-c4ccccc4)nc(-c4ccc5cccc(-c6cccc7ccccc67)c5c4)n3)cc2)cc1 ; c1ccc(-c2ccccc2-c2nc(-c3ccc4c(c3)oc3ccccc34)nc(-c3ccc4cccc(-c5cccc6ccccc56)c4c3)n2)cc1]

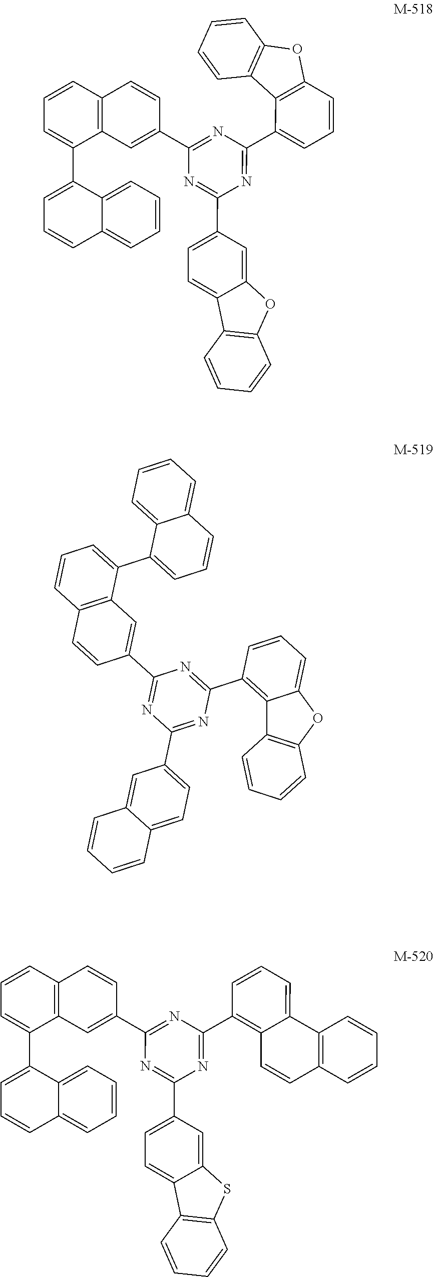 [SMILES: c1ccc2c(-c3cccc4ccc(-c5nc(-c6ccc7c(c6)oc6ccccc67)nc(-c6cccc7oc8ccccc8c67)n5)cc34)cccc2c1 ; c1ccc2cc(-c3nc(-c4ccc5cccc(-c6cccc7ccccc67)c5c4)nc(-c4cccc5oc6ccccc6c45)n3)ccc2c1 ; c1ccc2c(-c3cccc4ccc(-c5nc(-c6ccc7c(c6)sc6ccccc67)nc(-c6cccc7c6ccc6ccccc67)n5)cc34)cccc2c1]

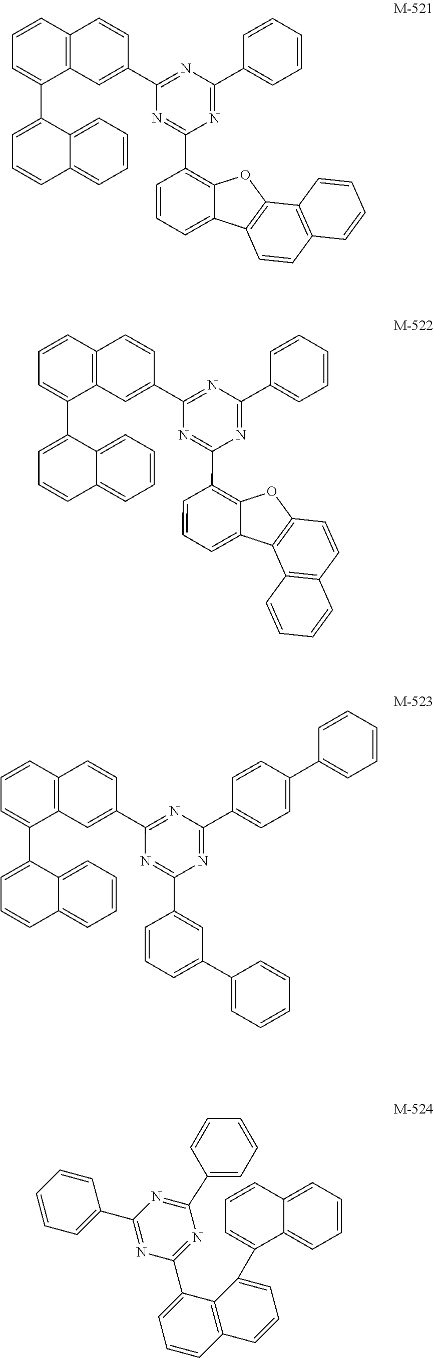 [SMILES: c1ccc(-c2nc(-c3ccc4cccc(-c5cccc6ccccc56)c4c3)nc(-c3cccc4c3oc3c5ccccc5ccc43)n2)cc1 ; c1ccc(-c2nc(-c3ccc4cccc(-c5cccc6ccccc56)c4c3)nc(-c3cccc4c3oc3ccc5ccccc5c34)n2)cc1 ; c1ccc(-c2ccc(-c3nc(-c4cccc(-c5ccccc5)c4)nc(-c4ccc5cccc(-c6cccc7ccccc67)c5c4)n3)cc2)cc1 ; c1ccc(-c2nc(-c3ccccc3)nc(-c3cccc4cccc(-c5cccc6ccccc56)c34)n2)cc1]

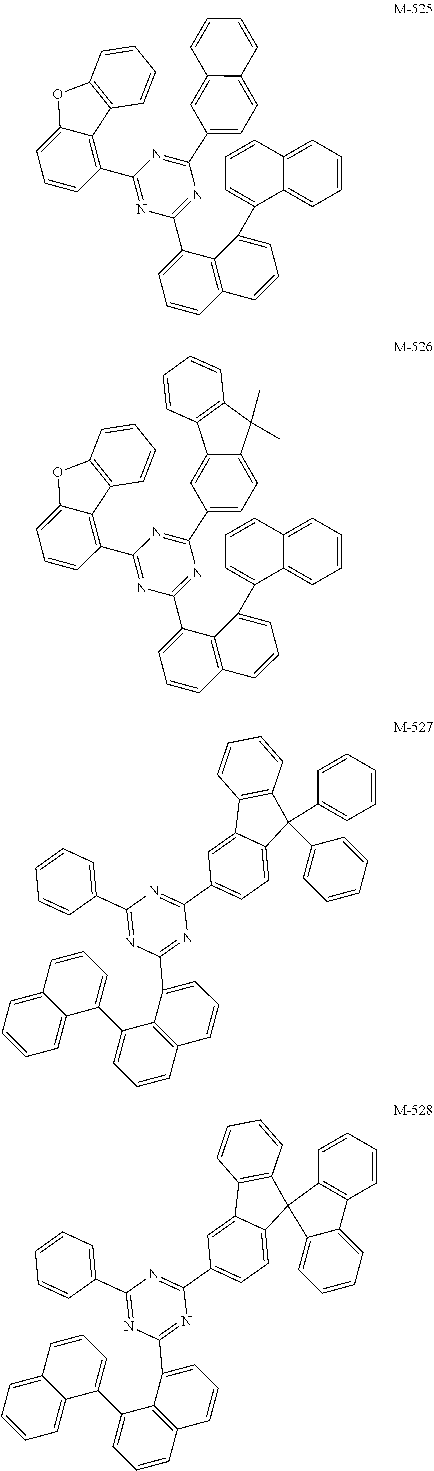

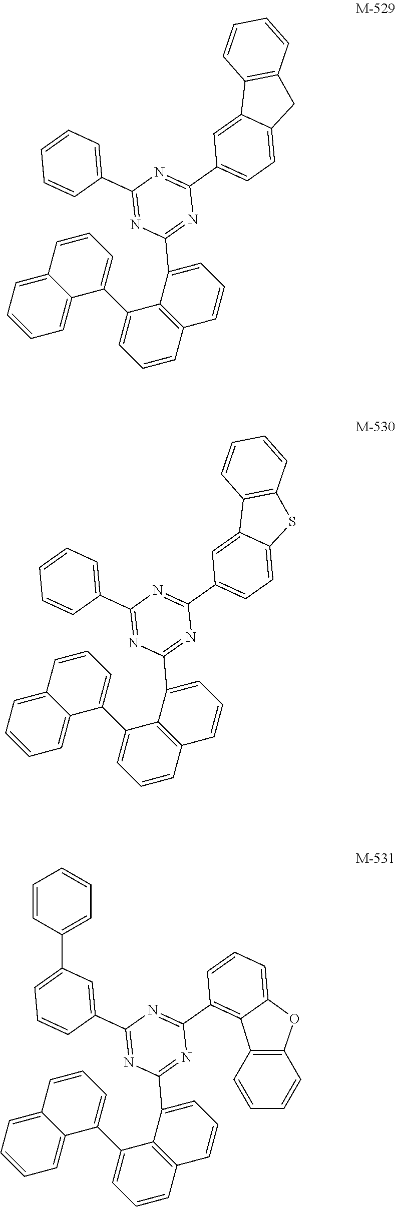 [SMILES: c1ccc(-c2nc(-c3ccc4c(c3)-c3ccccc3C4)nc(-c3cccc4cccc(-c5cccc6ccccc56)c34)n2)cc1 ; c1ccc(-c2nc(-c3ccc4sc5ccccc5c4c3)nc(-c3cccc4cccc(-c5cccc6ccccc56)c34)n2)cc1 ; c1ccc(-c2cccc(-c3nc(-c4cccc5cccc(-c6cccc7ccccc67)c45)nc(-c4cccc5oc6ccccc6c45)n3)c2)cc1]

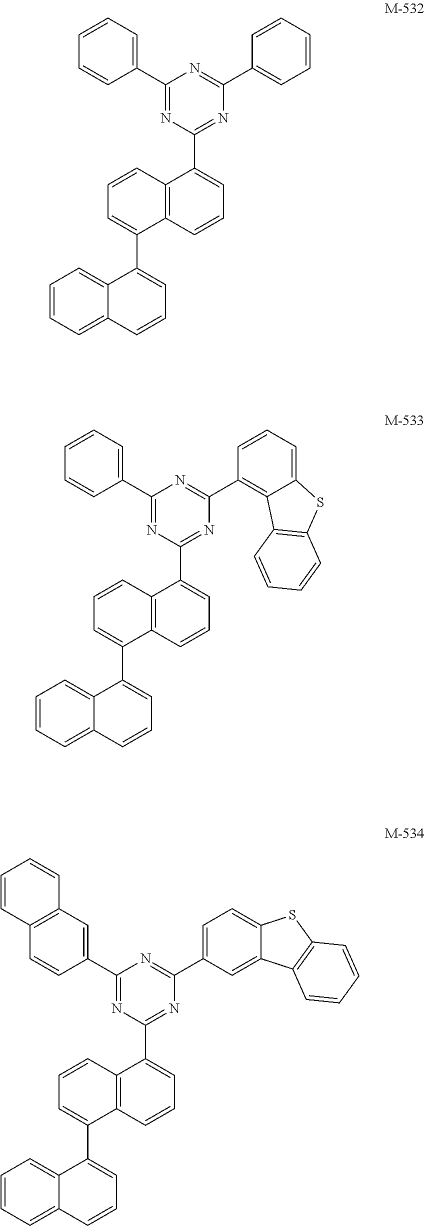 [SMILES: c1ccc(-c2nc(-c3ccccc3)nc(-c3cccc4c(-c5cccc6ccccc56)cccc34)n2)cc1 ; c1ccc(-c2nc(-c3cccc4c(-c5cccc6ccccc56)cccc34)nc(-c3cccc4sc5ccccc5c34)n2)cc1 ; c1ccc2cc(-c3nc(-c4ccc5sc6ccccc6c5c4)nc(-c4cccc5c(-c6cccc7ccccc67)cccc45)n3)ccc2c1]

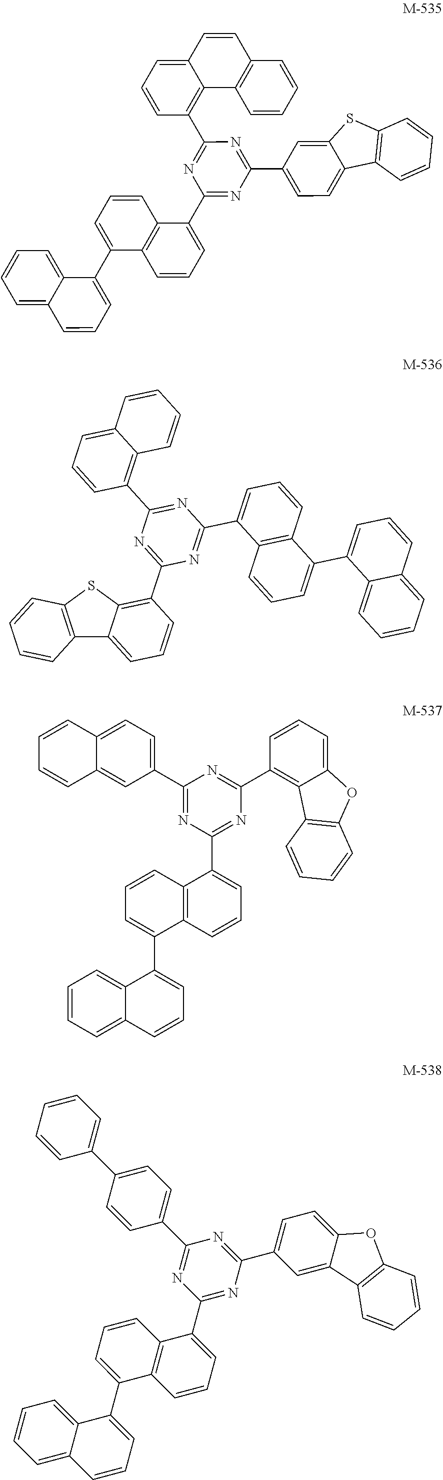 [SMILES: c1ccc2c(-c3cccc4c(-c5nc(-c6ccc7c(c6)sc6ccccc67)nc(-c6cccc7ccc8ccccc8c67)n5)cccc34)cccc2c1 ; c1ccc2c(-c3nc(-c4cccc5c(-c6cccc7ccccc67)cccc45)nc(-c4cccc5c4sc4ccccc45)n3)cccc2c1 ; c1ccc2cc(-c3nc(-c4cccc5c(-c6cccc7ccccc67)cccc45)nc(-c4cccc5oc6ccccc6c45)n3)ccc2c1 ; c1ccc(-c2ccc(-c3nc(-c4ccc5oc6ccccc6c5c4)nc(-c4cccc5c(-c6cccc7ccccc67)cccc45)n3)cc2)cc1]

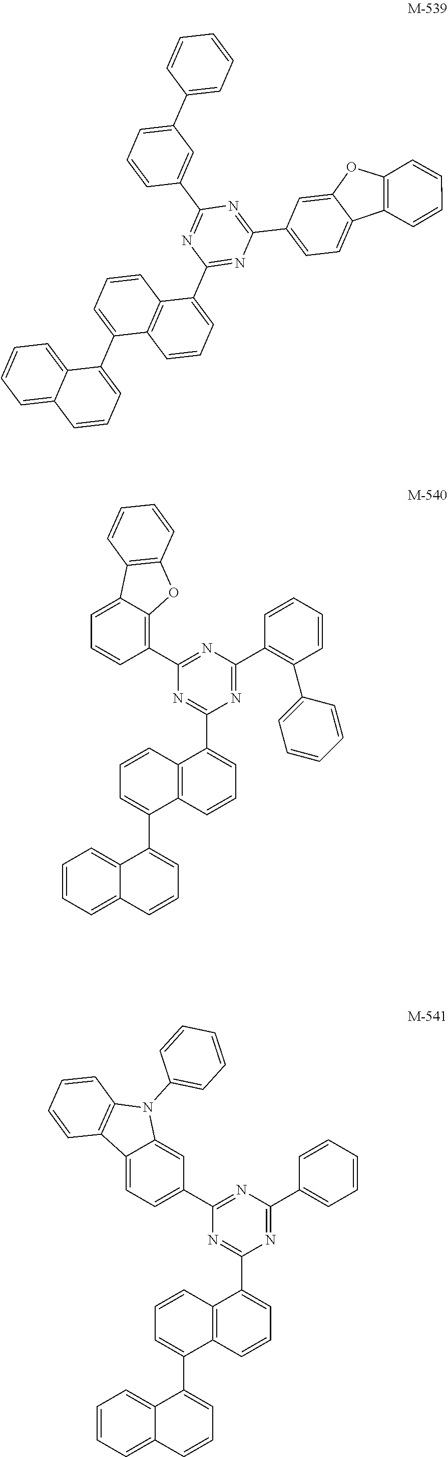 [SMILES: c1ccc(-c2cccc(-c3nc(-c4ccc5c(c4)oc4ccccc45)nc(-c4cccc5c(-c6cccc7ccccc67)cccc45)n3)c2)cc1 ; c1ccc(-c2ccccc2-c2nc(-c3cccc4c(-c5cccc6ccccc56)cccc34)nc(-c3cccc4c3oc3ccccc34)n2)cc1 ; c1ccc(-c2nc(-c3ccc4c5ccccc5n(-c5ccccc5)c4c3)nc(-c3cccc4c(-c5cccc6ccccc56)cccc34)n2)cc1]

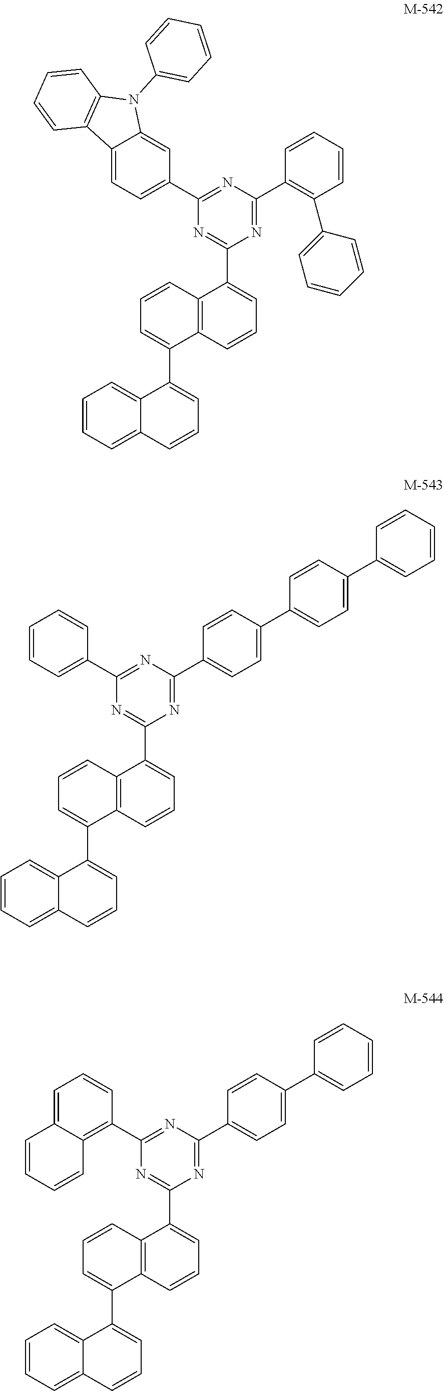 [SMILES: c1ccc(-c2ccccc2-c2nc(-c3ccc4c5ccccc5n(-c5ccccc5)c4c3)nc(-c3cccc4c(-c5cccc6ccccc56)cccc34)n2)cc1 ; c1ccc(-c2ccc(-c3ccc(-c4nc(-c5ccccc5)nc(-c5cccc6c(-c7cccc8ccccc78)cccc56)n4)cc3)cc2)cc1 ; c1ccc(-c2ccc(-c3nc(-c4cccc5ccccc45)nc(-c4cccc5c(-c6cccc7ccccc67)cccc45)n3)cc2)cc1]

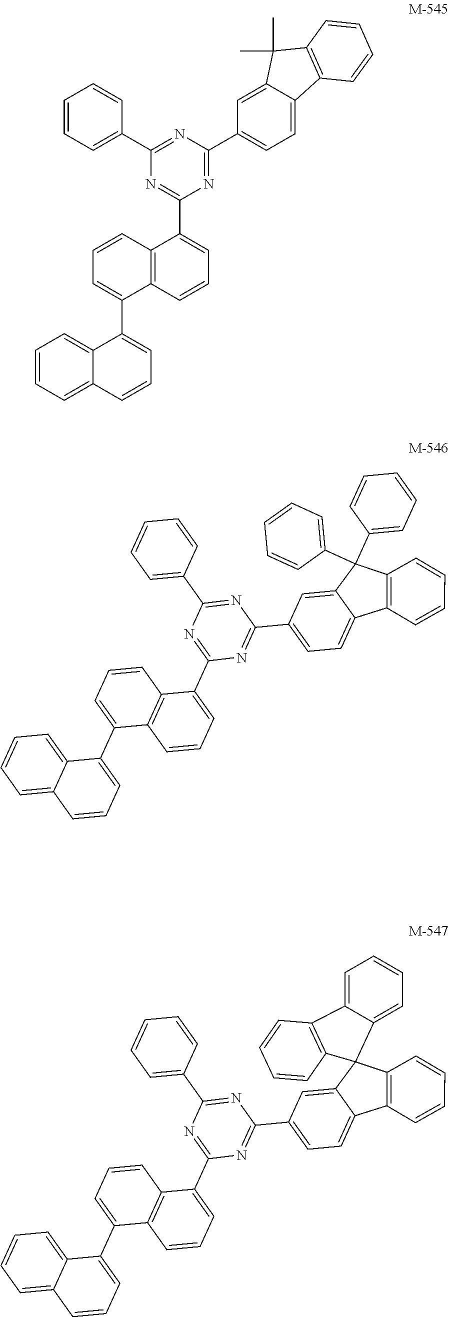 [SMILES: CC1(C)c2ccccc2-c2ccc(-c3nc(-c4ccccc4)nc(-c4cccc5c(-c6cccc7ccccc67)cccc45)n3)cc21 ; c1ccc(-c2nc(-c3ccc4c(c3)C(c3ccccc3)(c3ccccc3)c3ccccc3-4)nc(-c3cccc4c(-c5cccc6ccccc56)cccc34)n2)cc1 ; c1ccc(-c2nc(-c3ccc4c(c3)C3(c5ccccc5-c5ccccc53)c3ccccc3-4)nc(-c3cccc4c(-c5cccc6ccccc56)cccc34)n2)cc1]

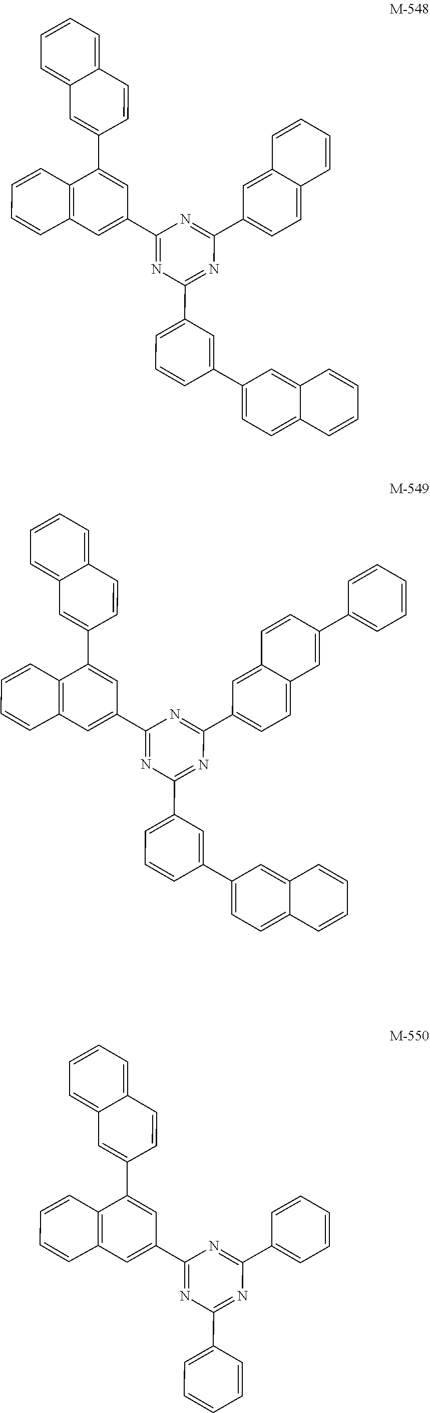 [SMILES: c1cc(-c2ccc3ccccc3c2)cc(-c2nc(-c3ccc4ccccc4c3)nc(-c3cc(-c4ccc5ccccc5c4)c4ccccc4c3)n2)c1 ; c1ccc(-c2ccc3cc(-c4nc(-c5cccc(-c6ccc7ccccc7c6)c5)nc(-c5cc(-c6ccc7ccccc7c6)c6ccccc6c5)n4)ccc3c2)cc1 ; c1ccc(-c2nc(-c3ccccc3)nc(-c3cc(-c4ccc5ccccc5c4)c4ccccc4c3)n2)cc1]

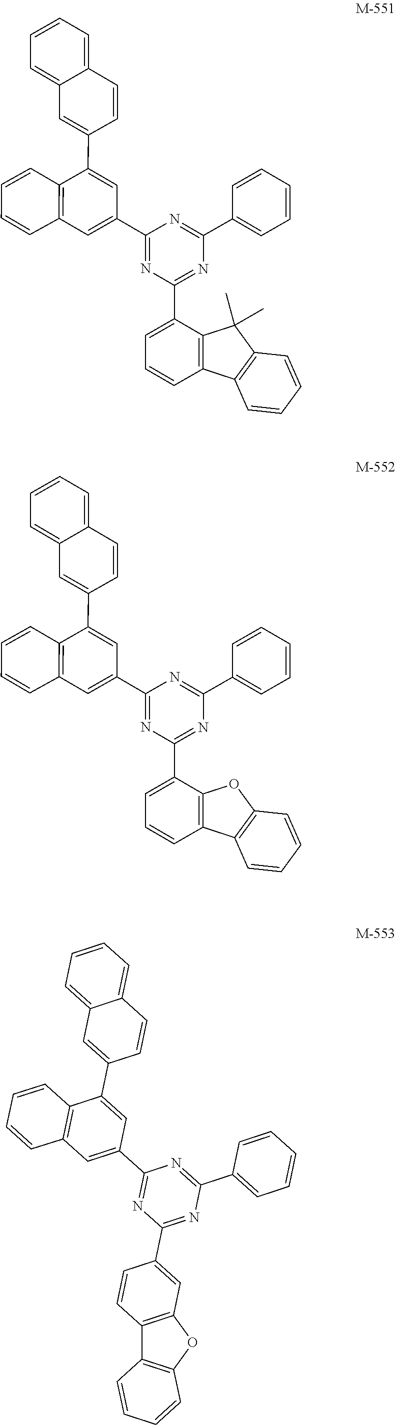 [SMILES: CC1(C)c2ccccc2-c2cccc(-c3nc(-c4ccccc4)nc(-c4cc(-c5ccc6ccccc6c5)c5ccccc5c4)n3)c21 ; c1ccc(-c2nc(-c3cc(-c4ccc5ccccc5c4)c4ccccc4c3)nc(-c3cccc4c3oc3ccccc34)n2)cc1 ; c1ccc(-c2nc(-c3cc(-c4ccc5ccccc5c4)c4ccccc4c3)nc(-c3ccc4c(c3)oc3ccccc34)n2)cc1]

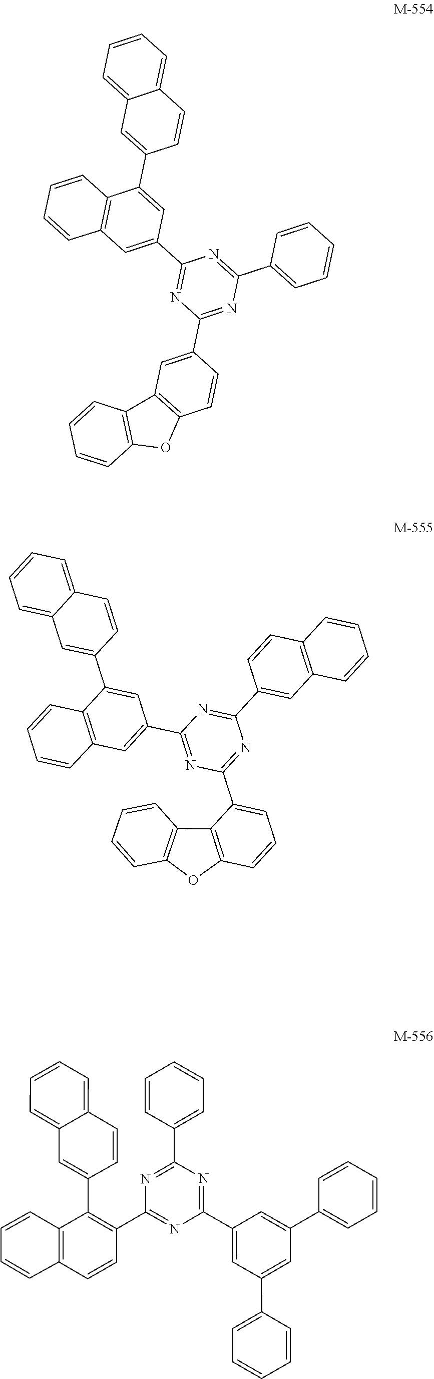 [SMILES: c1ccc(-c2nc(-c3cc(-c4ccc5ccccc5c4)c4ccccc4c3)nc(-c3ccc4oc5ccccc5c4c3)n2)cc1 ; c1ccc2cc(-c3nc(-c4cc(-c5ccc6ccccc6c5)c5ccccc5c4)nc(-c4cccc5oc6ccccc6c45)n3)ccc2c1 ; c1ccc(-c2cc(-c3ccccc3)cc(-c3nc(-c4ccccc4)nc(-c4ccc5ccccc5c4-c4ccc5ccccc5c4)n3)c2)cc1]

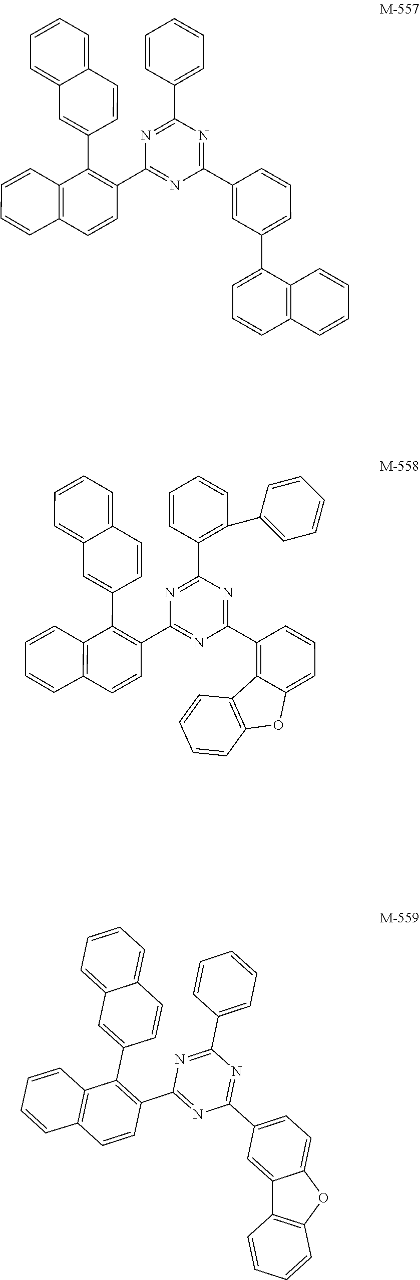 [SMILES: c1ccc(-c2nc(-c3cccc(-c4cccc5ccccc45)c3)nc(-c3ccc4ccccc4c3-c3ccc4ccccc4c3)n2)cc1 ; c1ccc(-c2ccccc2-c2nc(-c3ccc4ccccc4c3-c3ccc4ccccc4c3)nc(-c3cccc4oc5ccccc5c34)n2)cc1 ; c1ccc(-c2nc(-c3ccc4oc5ccccc5c4c3)nc(-c3ccc4ccccc4c3-c3ccc4ccccc4c3)n2)cc1]

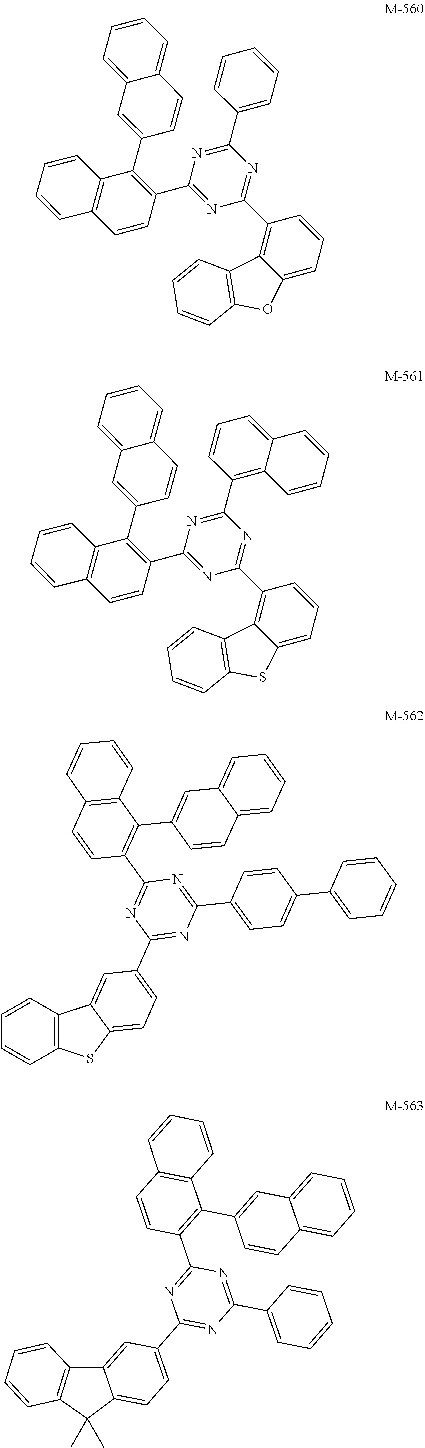 [SMILES: c1ccc(-c2nc(-c3ccc4ccccc4c3-c3ccc4ccccc4c3)nc(-c3cccc4oc5ccccc5c34)n2)cc1 ; c1ccc2cc(-c3c(-c4nc(-c5cccc6ccccc56)nc(-c5cccc6sc7ccccc7c56)n4)ccc4ccccc34)ccc2c1 ; c1ccc(-c2ccc(-c3nc(-c4ccc5sc6ccccc6c5c4)nc(-c4ccc5ccccc5c4-c4ccc5ccccc5c4)n3)cc2)cc1 ; CC1(C)c2ccccc2-c2cc(-c3nc(-c4ccccc4)nc(-c4ccc5ccccc5c4-c4ccc5ccccc5c4)n3)ccc21]

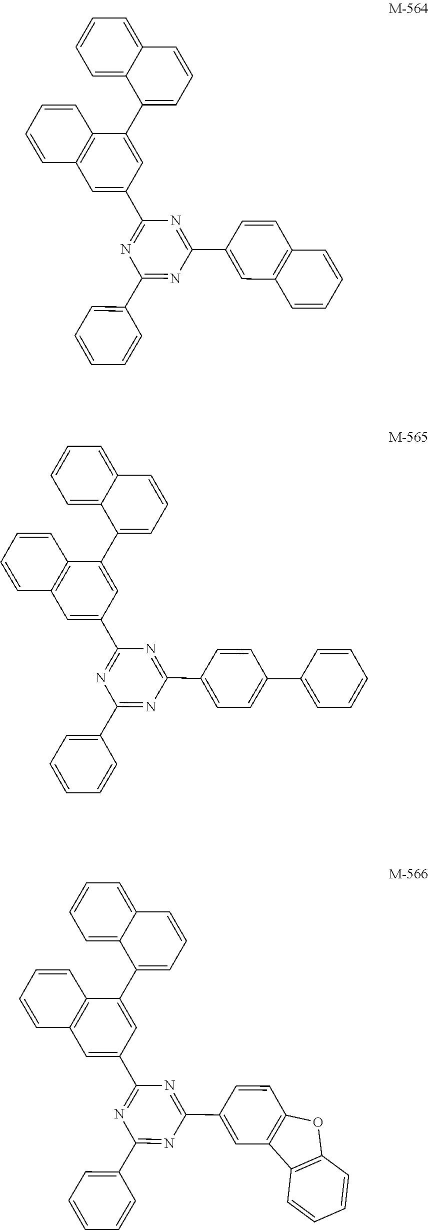 [SMILES: c1ccc(-c2nc(-c3ccc4ccccc4c3)nc(-c3cc(-c4cccc5ccccc45)c4ccccc4c3)n2)cc1 ; c1ccc(-c2ccc(-c3nc(-c4ccccc4)nc(-c4cc(-c5cccc6ccccc56)c5ccccc5c4)n3)cc2)cc1 ; c1ccc(-c2nc(-c3cc(-c4cccc5ccccc45)c4ccccc4c3)nc(-c3ccc4oc5ccccc5c4c3)n2)cc1]

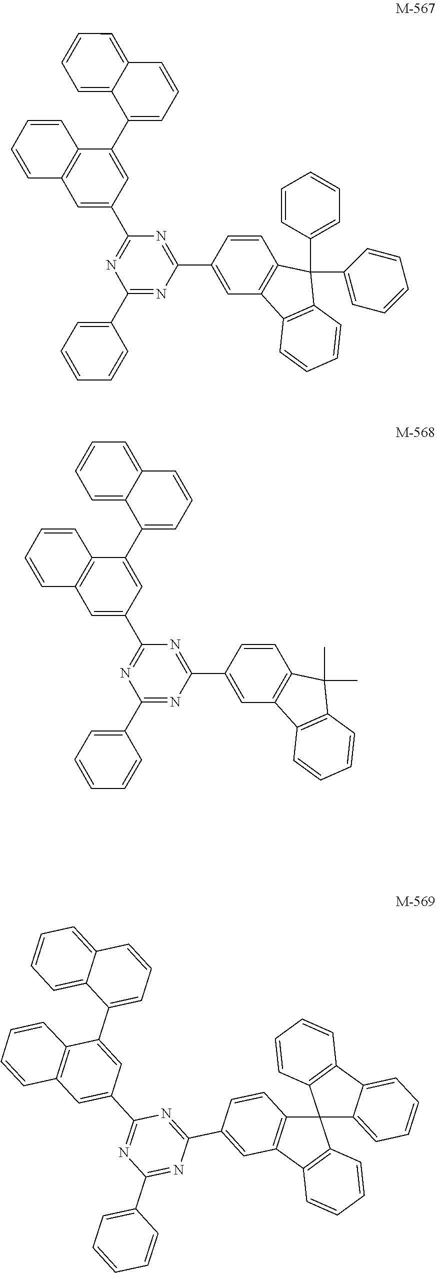 [SMILES: c1ccc(-c2nc(-c3ccc4c(c3)-c3ccccc3C4(c3ccccc3)c3ccccc3)nc(-c3cc(-c4cccc5ccccc45)c4ccccc4c3)n2)cc1 ; CC1(C)c2ccccc2-c2cc(-c3nc(-c4ccccc4)nc(-c4cc(-c5cccc6ccccc56)c5ccccc5c4)n3)ccc21 ; c1ccc(-c2nc(-c3ccc4c(c3)-c3ccccc3C43c4ccccc4-c4ccccc43)nc(-c3cc(-c4cccc5ccccc45)c4ccccc4c3)n2)cc1]

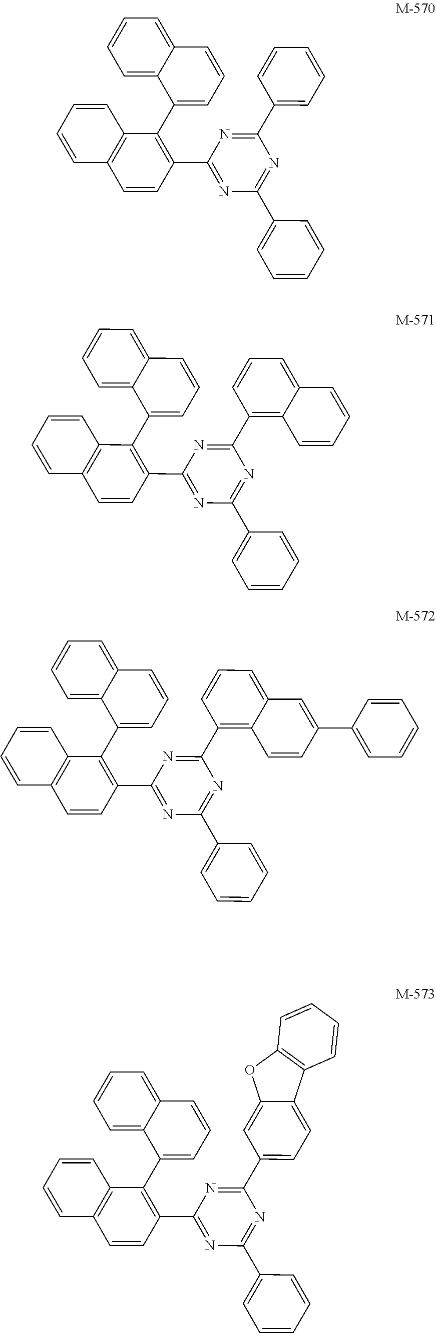 [SMILES: c1ccc(-c2nc(-c3ccccc3)nc(-c3ccc4ccccc4c3-c3cccc4ccccc34)n2)cc1 ; c1ccc(-c2nc(-c3ccc4ccccc4c3-c3cccc4ccccc34)nc(-c3cccc4ccccc34)n2)cc1 ; c1ccc(-c2ccc3c(-c4nc(-c5ccccc5)nc(-c5ccc6ccccc6c5-c5cccc6ccccc56)n4)cccc3c2)cc1 ; c1ccc(-c2nc(-c3ccc4c(c3)oc3ccccc34)nc(-c3ccc4ccccc4c3-c3cccc4ccccc34)n2)cc1]

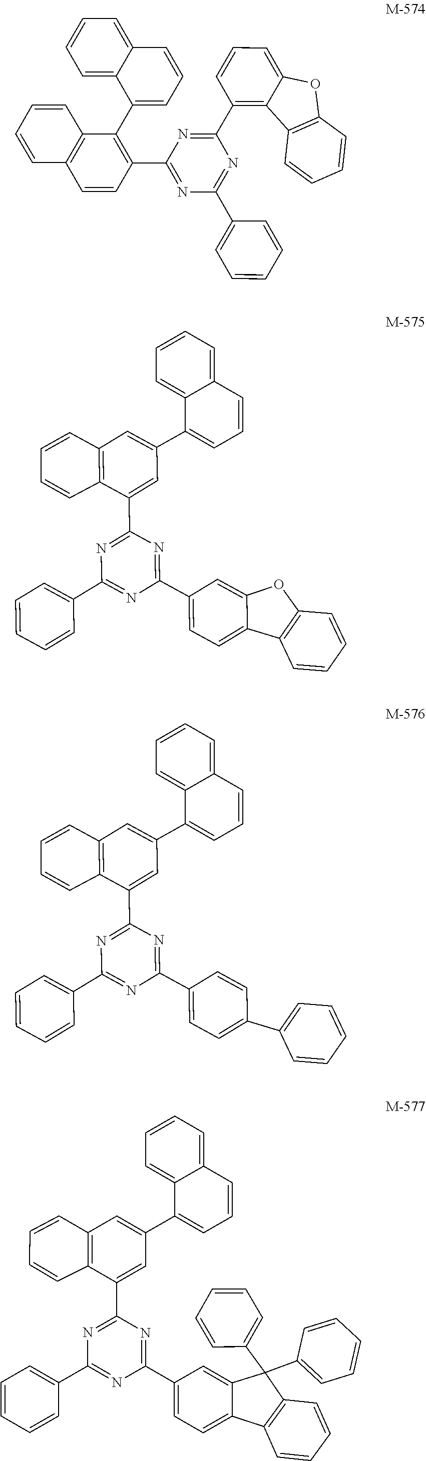 [SMILES: c1ccc(-c2nc(-c3ccc4ccccc4c3-c3cccc4ccccc34)nc(-c3cccc4oc5ccccc5c34)n2)cc1 ; c1ccc(-c2nc(-c3ccc4c(c3)oc3ccccc34)nc(-c3cc(-c4cccc5ccccc45)cc4ccccc34)n2)cc1 ; c1ccc(-c2ccc(-c3nc(-c4ccccc4)nc(-c4cc(-c5cccc6ccccc56)cc5ccccc45)n3)cc2)cc1 ; c1ccc(-c2nc(-c3ccc4c(c3)C(c3ccccc3)(c3ccccc3)c3ccccc3-4)nc(-c3cc(-c4cccc5ccccc45)cc4ccccc34)n2)cc1]

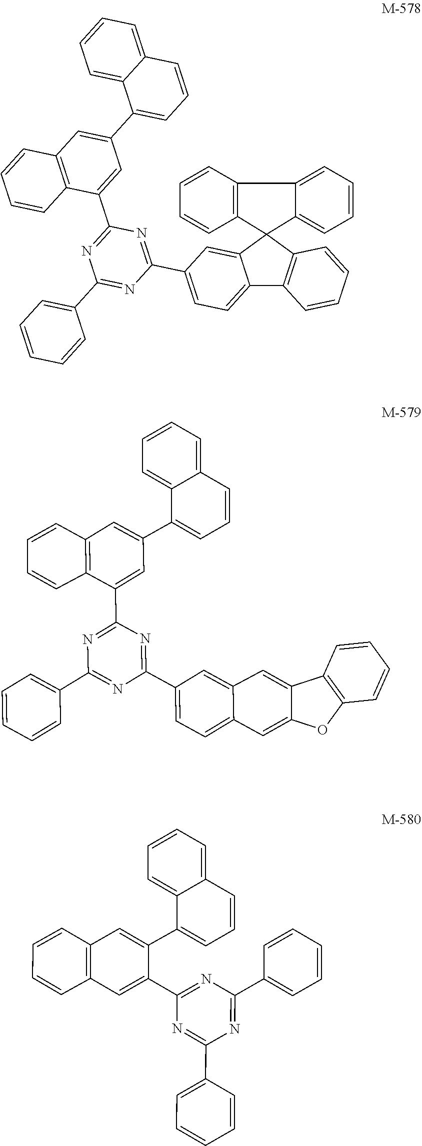 [SMILES: c1ccc(-c2nc(-c3ccc4c(c3)C3(c5ccccc5-c5ccccc53)c3ccccc3-4)nc(-c3cc(-c4cccc5ccccc45)cc4ccccc34)n2)cc1 ; c1ccc(-c2nc(-c3ccc4cc5oc6ccccc6c5cc4c3)nc(-c3cc(-c4cccc5ccccc45)cc4ccccc34)n2)cc1 ; c1ccc(-c2nc(-c3ccccc3)nc(-c3cc4ccccc4cc3-c3cccc4ccccc34)n2)cc1]

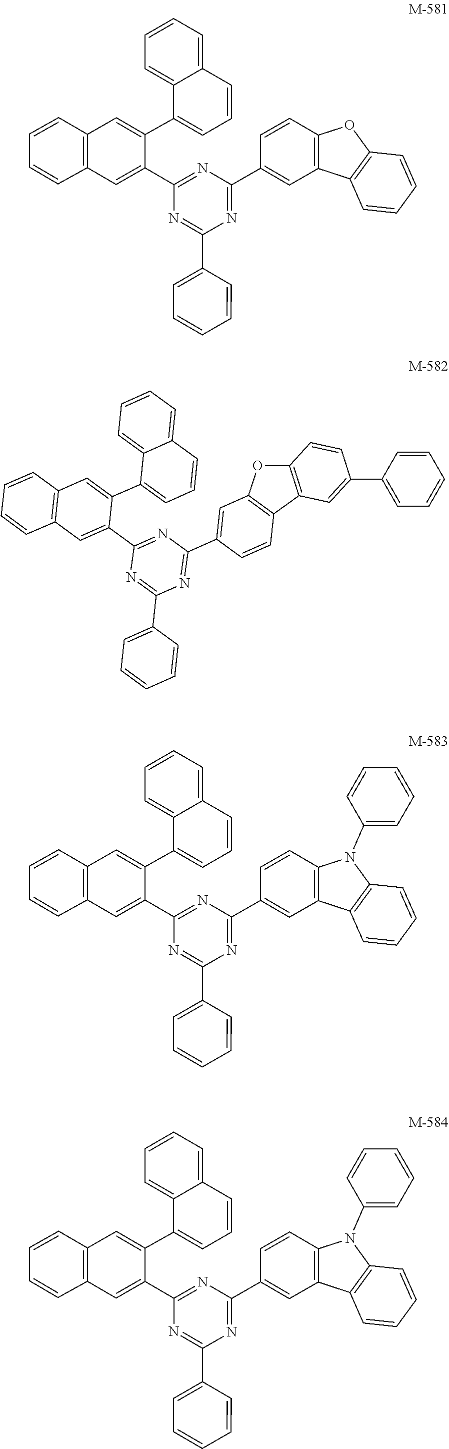 [SMILES: c1ccc(-c2nc(-c3ccc4oc5ccccc5c4c3)nc(-c3cc4ccccc4cc3-c3cccc4ccccc34)n2)cc1 ; c1ccc(-c2ccc3oc4cc(-c5nc(-c6ccccc6)nc(-c6cc7ccccc7cc6-c6cccc7ccccc67)n5)ccc4c3c2)cc1 ; c1ccc(-c2nc(-c3ccc4c(c3)c3ccccc3n4-c3ccccc3)nc(-c3cc4ccccc4cc3-c3cccc4ccccc34)n2)cc1 ; c1ccc(-c2nc(-c3ccc4c(c3)c3ccccc3n4-c3ccccc3)nc(-c3cc4ccccc4cc3-c3cccc4ccccc34)n2)cc1]

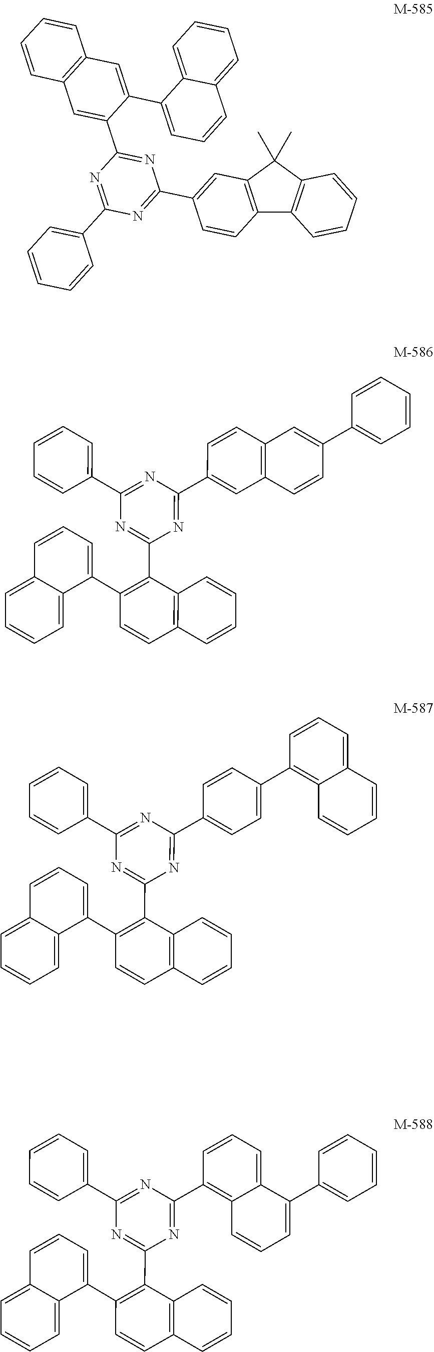 [SMILES: CC1(C)c2ccccc2-c2ccc(-c3nc(-c4ccccc4)nc(-c4cc5ccccc5cc4-c4cccc5ccccc45)n3)cc21 ; c1ccc(-c2ccc3cc(-c4nc(-c5ccccc5)nc(-c5c(-c6cccc7ccccc67)ccc6ccccc56)n4)ccc3c2)cc1 ; c1ccc(-c2nc(-c3ccc(-c4cccc5ccccc45)cc3)nc(-c3c(-c4cccc5ccccc45)ccc4ccccc34)n2)cc1 ; c1ccc(-c2nc(-c3c(-c4cccc5ccccc45)ccc4ccccc34)nc(-c3cccc4c(-c5ccccc5)cccc34)n2)cc1]

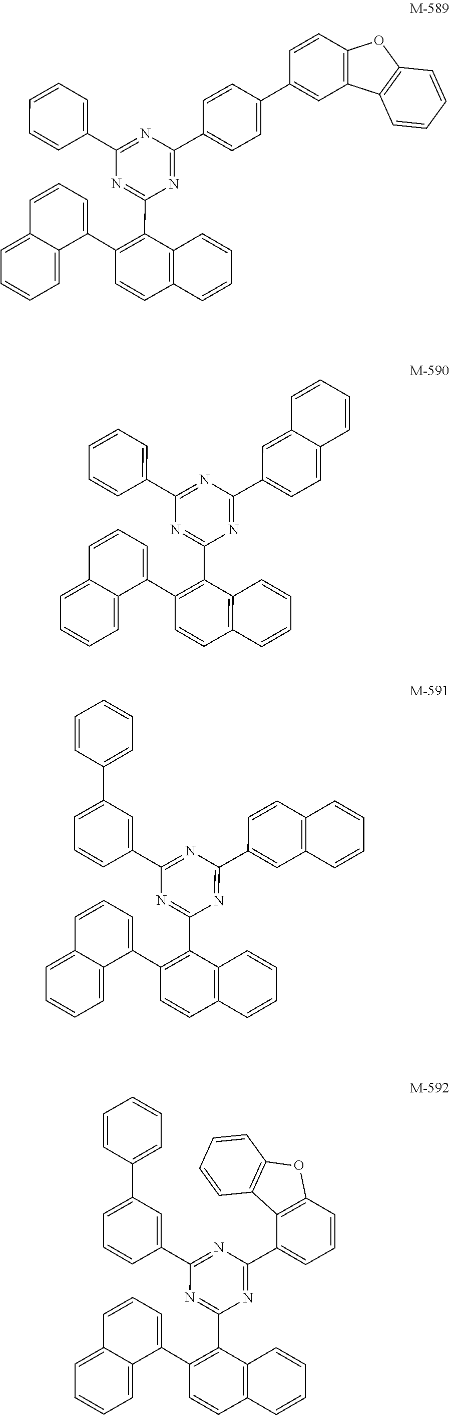 [SMILES: c1ccc(-c2nc(-c3ccc(-c4ccc5oc6ccccc6c5c4)cc3)nc(-c3c(-c4cccc5ccccc45)ccc4ccccc34)n2)cc1 ; c1ccc(-c2nc(-c3ccc4ccccc4c3)nc(-c3c(-c4cccc5ccccc45)ccc4ccccc34)n2)cc1 ; c1ccc(-c2cccc(-c3nc(-c4ccc5ccccc5c4)nc(-c4c(-c5cccc6ccccc56)ccc5ccccc45)n3)c2)cc1 ; c1ccc(-c2cccc(-c3nc(-c4c(-c5cccc6ccccc56)ccc5ccccc45)nc(-c4cccc5oc6ccccc6c45)n3)c2)cc1]

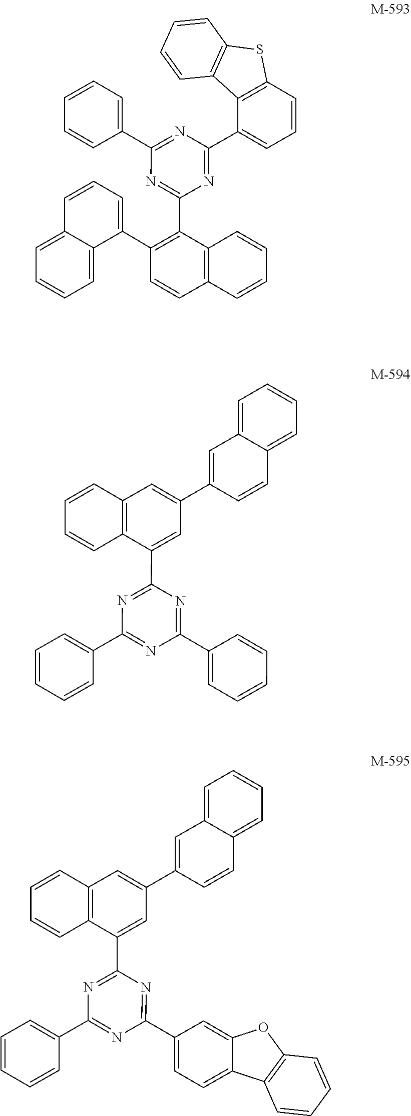 [SMILES: c1ccc(-c2nc(-c3c(-c4cccc5ccccc45)ccc4ccccc34)nc(-c3cccc4sc5ccccc5c34)n2)cc1 ; c1ccc(-c2nc(-c3ccccc3)nc(-c3cc(-c4ccc5ccccc5c4)cc4ccccc34)n2)cc1 ; c1ccc(-c2nc(-c3ccc4c(c3)oc3ccccc34)nc(-c3cc(-c4ccc5ccccc5c4)cc4ccccc34)n2)cc1]

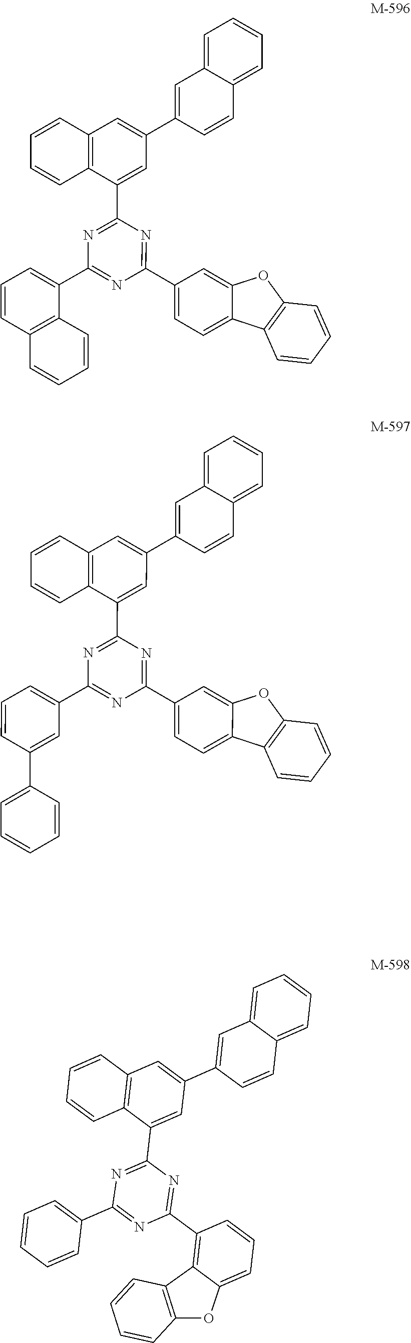 [SMILES: c1ccc2cc(-c3cc(-c4nc(-c5ccc6c(c5)oc5ccccc56)nc(-c5cccc6ccccc56)n4)c4ccccc4c3)ccc2c1 ; c1ccc(-c2cccc(-c3nc(-c4ccc5c(c4)oc4ccccc45)nc(-c4cc(-c5ccc6ccccc6c5)cc5ccccc45)n3)c2)cc1 ; c1ccc(-c2nc(-c3cc(-c4ccc5ccccc5c4)cc4ccccc34)nc(-c3cccc4oc5ccccc5c34)n2)cc1]

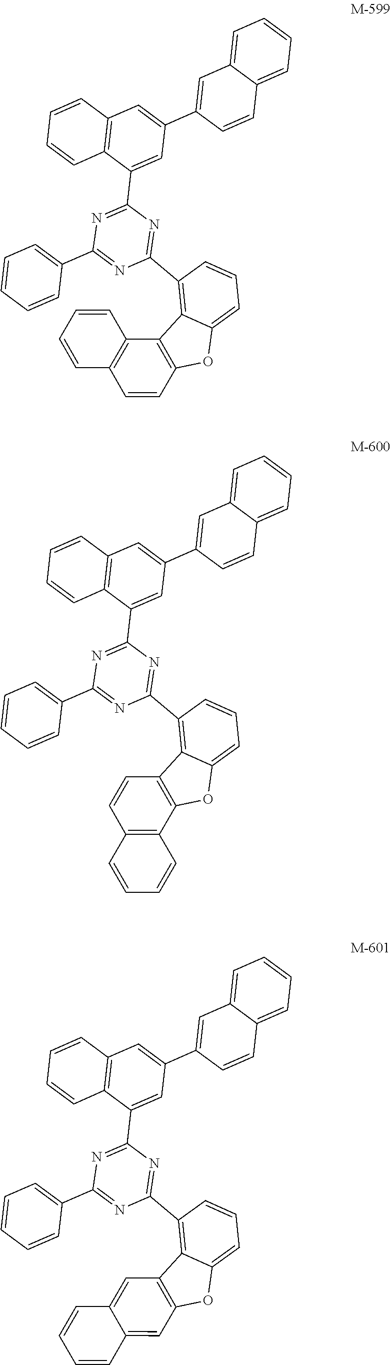 [SMILES: c1ccc(-c2nc(-c3cc(-c4ccc5ccccc5c4)cc4ccccc34)nc(-c3cccc4oc5ccc6ccccc6c5c34)n2)cc1 ; c1ccc(-c2nc(-c3cc(-c4ccc5ccccc5c4)cc4ccccc34)nc(-c3cccc4oc5c6ccccc6ccc5c34)n2)cc1 ; c1ccc(-c2nc(-c3cc(-c4ccc5ccccc5c4)cc4ccccc34)nc(-c3cccc4oc5cc6ccccc6cc5c34)n2)cc1]

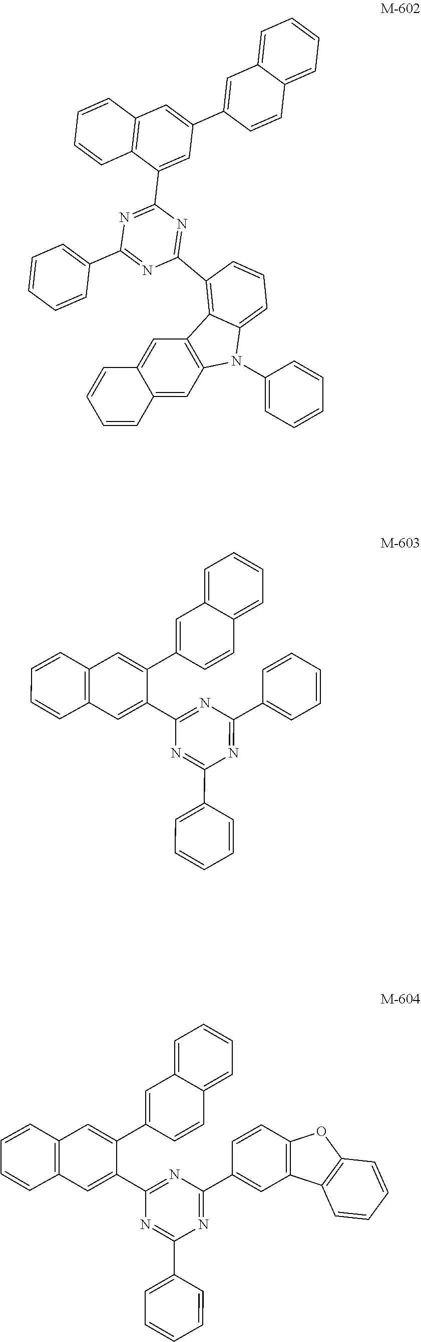 [SMILES: c1ccc(-c2nc(-c3cc(-c4ccc5ccccc5c4)cc4ccccc34)nc(-c3cccc4c3c3cc5ccccc5cc3n4-c3ccccc3)n2)cc1 ; c1ccc(-c2nc(-c3ccccc3)nc(-c3cc4ccccc4cc3-c3ccc4ccccc4c3)n2)cc1 ; c1ccc(-c2nc(-c3ccc4oc5ccccc5c4c3)nc(-c3cc4ccccc4cc3-c3ccc4ccccc4c3)n2)cc1]

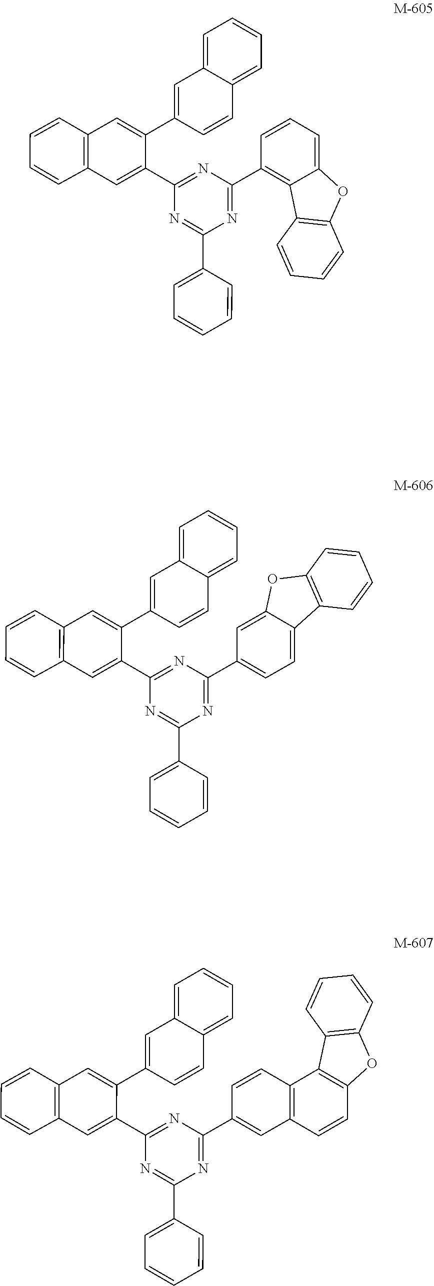 [SMILES: c1ccc(-c2nc(-c3cc4ccccc4cc3-c3ccc4ccccc4c3)nc(-c3cccc4oc5ccccc5c34)n2)cc1 ; c1ccc(-c2nc(-c3ccc4c(c3)oc3ccccc34)nc(-c3cc4ccccc4cc3-c3ccc4ccccc4c3)n2)cc1 ; c1ccc(-c2nc(-c3ccc4c(ccc5oc6ccccc6c54)c3)nc(-c3cc4ccccc4cc3-c3ccc4ccccc4c3)n2)cc1]

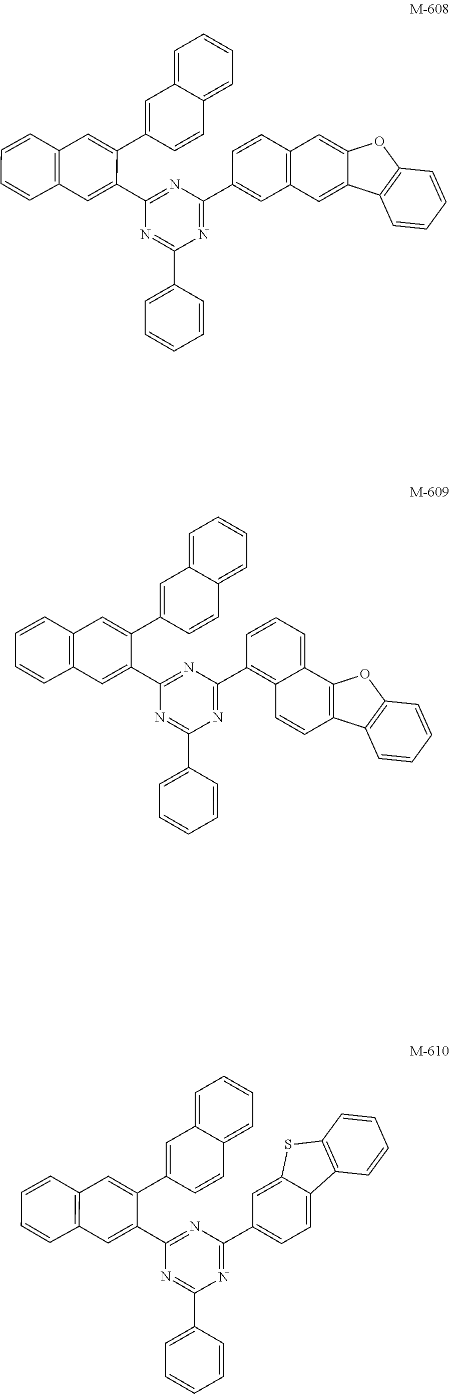 [SMILES: c1ccc(-c2nc(-c3ccc4cc5oc6ccccc6c5cc4c3)nc(-c3cc4ccccc4cc3-c3ccc4ccccc4c3)n2)cc1 ; c1ccc(-c2nc(-c3cc4ccccc4cc3-c3ccc4ccccc4c3)nc(-c3cccc4c3ccc3c5ccccc5oc43)n2)cc1 ; c1ccc(-c2nc(-c3ccc4c(c3)sc3ccccc34)nc(-c3cc4ccccc4cc3-c3ccc4ccccc4c3)n2)cc1]

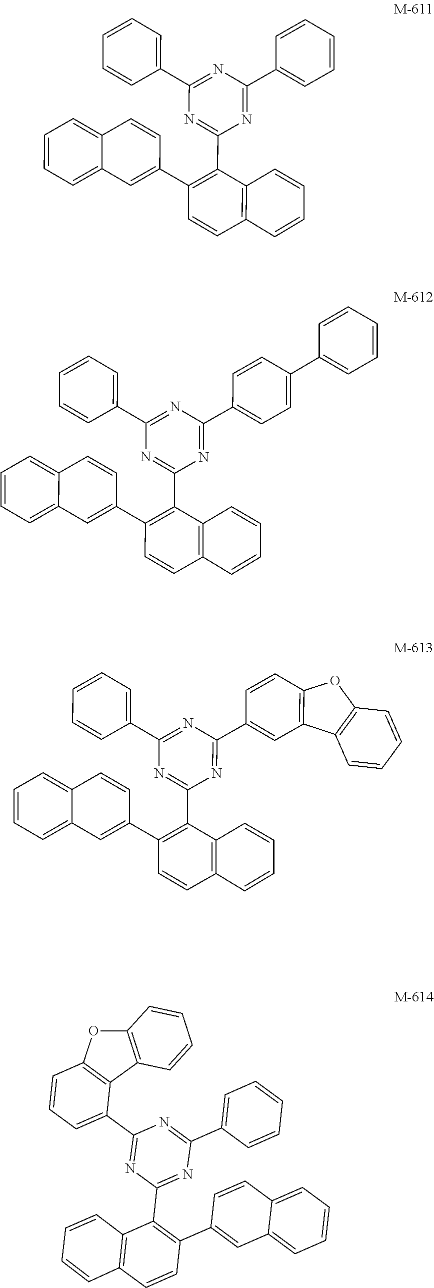 [SMILES: c1ccc(-c2nc(-c3ccccc3)nc(-c3c(-c4ccc5ccccc5c4)ccc4ccccc34)n2)cc1 ; c1ccc(-c2ccc(-c3nc(-c4ccccc4)nc(-c4c(-c5ccc6ccccc6c5)ccc5ccccc45)n3)cc2)cc1 ; c1ccc(-c2nc(-c3ccc4oc5ccccc5c4c3)nc(-c3c(-c4ccc5ccccc5c4)ccc4ccccc34)n2)cc1 ; c1ccc(-c2nc(-c3c(-c4ccc5ccccc5c4)ccc4ccccc34)nc(-c3cccc4oc5ccccc5c34)n2)cc1]

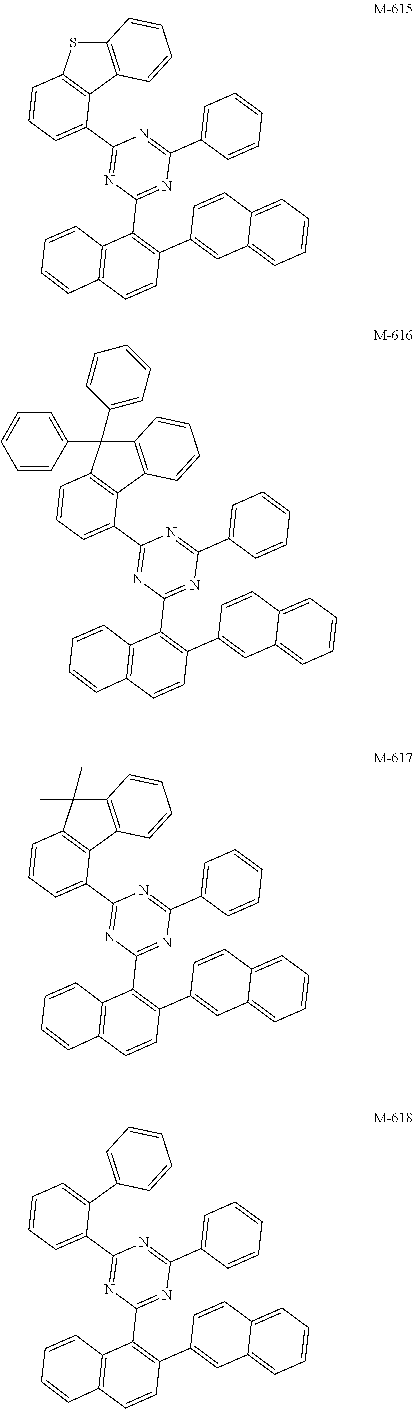

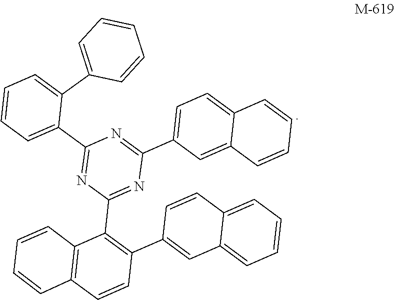 [SMILES: c1ccc(-c2ccccc2-c2nc(-c3ccc4ccccc4c3)nc(-c3c(-c4ccc5ccccc5c4)ccc4ccccc34)n2)cc1]

The present application also provides an organic electroluminescent device, the organic electroluminescent device comprises a cathode, an anode, and an organic layer arranged between the cathode and the anode; the organic layer comprises the organic electroluminescent compound above.

Preferably, the organic layer further comprises the organic electroluminescent material composition above.

Preferably, the organic layer comprises a hole transport layer, and the hole transport layer comprises the organic electroluminescent compound above.

Preferably, the organic layer comprises a electron transport layer, and the electron transport layer further comprises the organic electroluminescent compound above.

Preferably, the organic layer comprises a light emitting layer, and the light emitting layer comprises the organic electroluminescent compound above.

Preferably, the light emitting layer comprises the organic electroluminescent material composition above.

Alternatively, the material of the light emitting layer comprises a host material and a guest material; the host material comprises the organic electroluminescent compound above.

In an embodiment of an organic electroluminescent device of the present application, the organic layer is composed of sequentially stacked hole injection layer, hole transport layer, light emitting layer, electron transport layer, and electron injection layer. The organic electroluminescent device further comprises a substrate located on the surface of the anode facing away from the cathode.

The present application also provides applications of the organic electroluminescent device above in fiber optic devices, lighting devices, electronic photographic photosensitive devices, photoelectric converters, organic solar cells, switching element devices, organic luminescent field-effect transistors, image sensors, or dye lasers.

The beneficial effects of the present application are as follows:

The organic electroluminescent compound provided by the present application is based on the structure of Formula (1) and further defines the structure of R. It has the characteristic of high carrier mobility, thereby improving the device's luminous efficiency. The HOMO and LUMO energy levels of the organic electroluminescent compound have a high degree of matching with adjacent energy levels, resulting in higher stability and better balanced carrier mobility of the organic electroluminescent compound. This in turn enables the organic electroluminescent device containing the organic electroluminescent compound to have lower driving voltage, higher luminous efficiency, and longer lifespan.

1. An organic electroluminescent compound, wherein the organic electroluminescent compound has a structure of the following Formula (1): 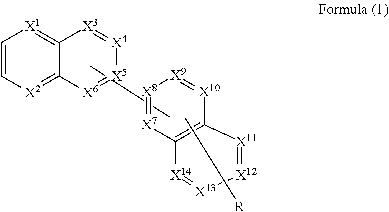
R is selected from the following structure: 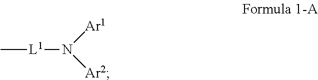
wherein, X¹-X¹⁴ are each independently selected from N or CR⁸, and R⁸ is selected from hydrogen or deuterium, wherein R⁸ is each present independently, and can be the same or different;
L¹ is selected from a connecting bond, a substituted or unsubstituted C6-C30 arylenyl, and a substituted or unsubstituted C3-C30 heteroarylenyl;
Ar¹ and Ar² are each independently selected from a substituted or unsubstituted C6-C30 aryl, a substituted or unsubstituted C3-C30 heteroaryl, a substituted or unsubstituted C6-C60 arylamine group, and a substituted or unsubstituted C3-C60 heteroarylamine group;
the substituents in the substituted C6-C30 arylenyl, the substituted C3-C30 heteroarylenyl, the substituted C6-C30 aryl, the substituted C3-C30 heteroaryl, the substituted C6-C60 arylamine group, and the substituted C3-C60 heteroarylamine group are selected from one of or a combination of two of deuterium, halogen, cyano, a C1-C6 alkyl, a C3-C30 cycloalkyl, a C6-C30 aryl, a C3-C30 heteroaryl, a C6-C60 arylamine group and a C3-C60 heteroarylamine group.

2. The organic electroluminescent compound according to claim 1, wherein in Formula (1), X¹-X¹⁴ are all selected from CR⁸, and R⁸ is selected from hydrogen or deuterium;
preferably, X¹-X¹⁴ are all selected from CR⁸, wherein R⁸ is hydrogen;
preferably, any one of X¹-X⁶ is selected from N, and the remaining is CR⁸;
preferably, any one of X¹-X⁶ is selected from N, and the remaining is CR⁸; any one of X⁷-X¹⁴ is selected from N, and the remaining is CR⁸;
wherein R⁸ is each present independently, and can be the same or different; and
preferably, Ar¹-Ar² are each independently selected from a substituted or unsubstituted A group;
the A group comprises: phenyl, naphthyl, biphenyl, triphenyl, phenanthryl, fluoranthenyl, triphenylenyl, fluorenyl, dimethylfluorenyl, diphenylfluorenyl, spirobifluorenyl, benzodimethylfluorenyl, benzodiphenylfluorenyl, benzospirobifluorenyl, benzofuryl, dibenzofuryl, naphthobenzofuryl, dinaphthalofuryl, benzothienyl, dibenzothienyl, naphthobenzothienyl, carbazolyl, phenylcarbazolyl, benzomethylcarbazolyl, dibenzocarbazolyl, biphenylcarbazolyl, phenanthrobenzofuryl, dibenzofuranofuryl, and phenylcarbazolobenzofuryl;
wherein, the substituent of the substituted A group is selected from one of or a combination of two of deuterium, halogen, cyano, a C1-C6 alkyl, a C3-C30 cycloalkyl, a C6-C30 aryl, a C3-C30 heteroaryl, a C6-C60 arylamine group, and a C3-C60 heteroarylamine group; and
preferably, L¹ is selected from a connecting bond, and a substituted or unsubstituted C6-C18 arylenyl.

3. The organic electroluminescent compound according to claim 1, wherein Ar¹-Ar² are each independently selected from phenyl, naphthyl, biphenyl, triphenyl, phenanthryl, fluoranthenyl, triphenylenyl, fluorenyl, dimethylfluorenyl, diphenylfluorenyl, spirobifluorenyl, benzodimethylfluorenyl, benzodiphenylfluorenyl, benzospirobifluorenyl, dibenzofuryl, naphthobenzofuryl, dibenzothienyl, naphthobenzothienyl, phenylcarbazolyl, benzophenylcarbazolyl, dibenzophenylcarbazolyl, or a group with the following structure: 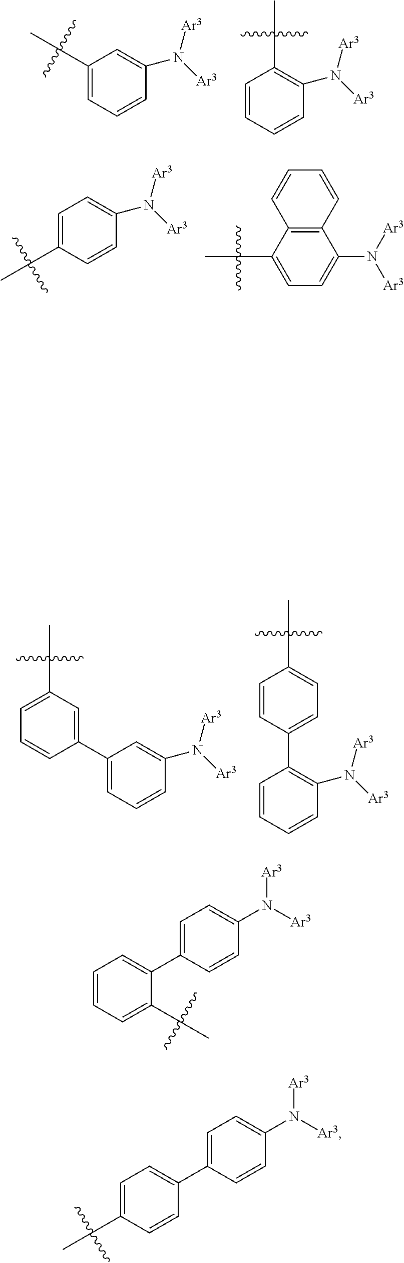
when a plurality of Ar³ are each present independently, the plurality of Ar³ can be the same or different;
wherein, Ar³ is each independently selected from a substituted or unsubstituted B group, and the B group comprises: phenyl, naphthyl, biphenyl, triphenyl, phenanthryl, fluoranthenyl, triphenylenyl, fluorenyl, dimethylfluorenyl, diphenylfluorenyl, spirobifluorenyl, benzodimethylfluorenyl, benzodiphenylfluorenyl, benzospirobifluorenyl, benzofuryl, dibenzofuryl, naphthobenzofuryl, benzothienyl, dibenzothienyl, naphthobenzothienyl, carbazolyl, phenylcarbazolyl, benzophenylcarbazolyl, and dibenzophenylcarbazolyl;
wherein, the substituent of the substituted B group is selected from one of or a combination of two of deuterium, halogen, cyano, a C1-C6 alkyl, a C3-C30 cycloalkyl, a C6-C30 aryl, a C3-C30 heteroaryl, a C6-C60 arylamine group, and a C3-C60 heteroarylamine group; and
preferably, L¹ is selected from a connecting bond, phenylenyl and naphthenyl.

4. The organic electroluminescent compound according to claim 1, wherein the structure of the organic electroluminescent compound is shown as any one of Formula 1-1 to Formula 1-64: 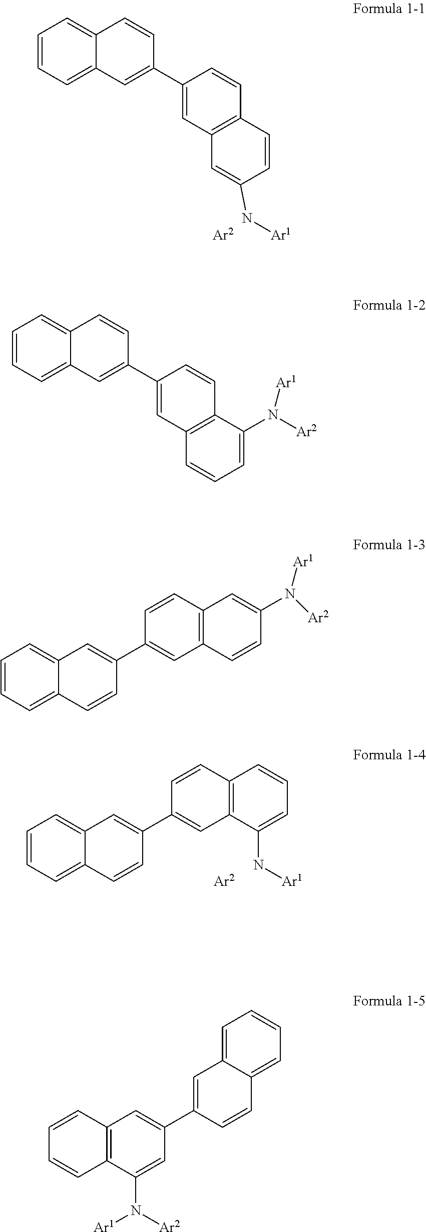 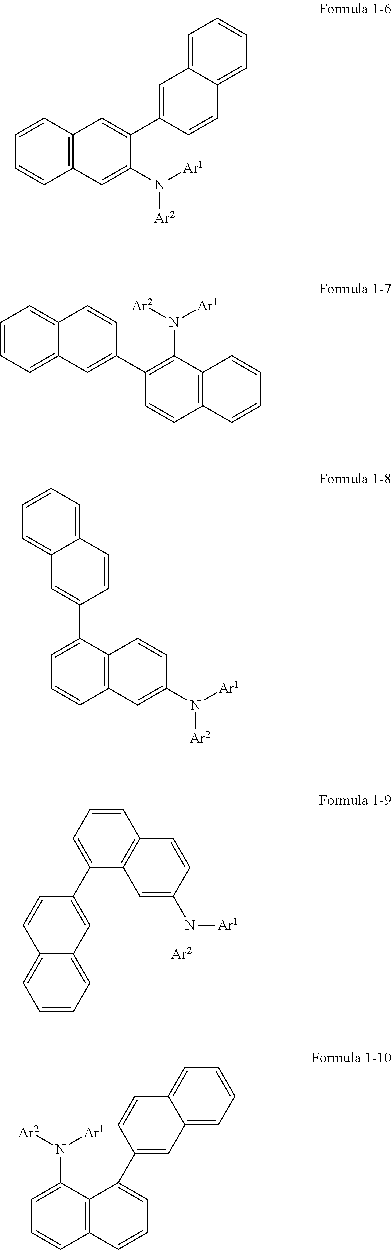 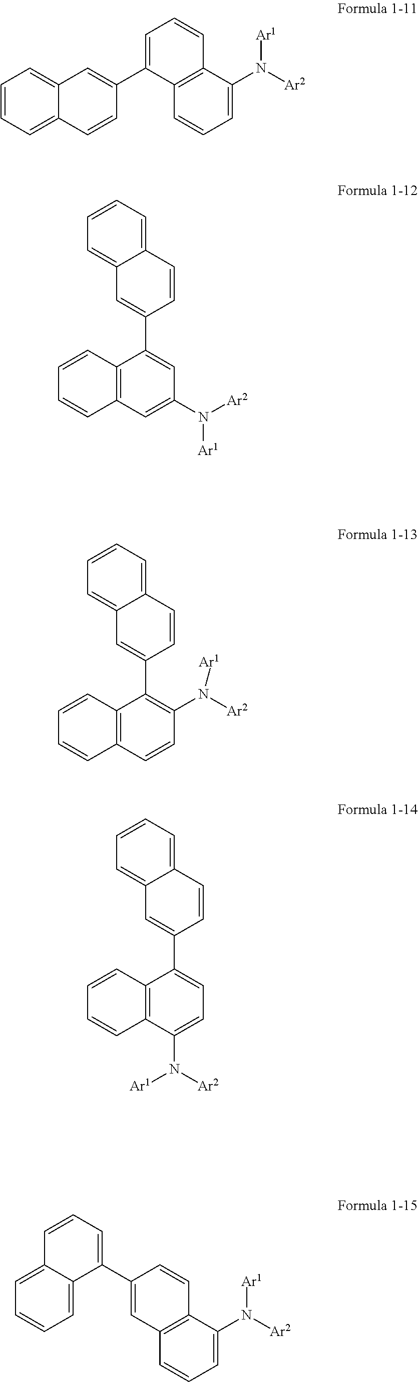 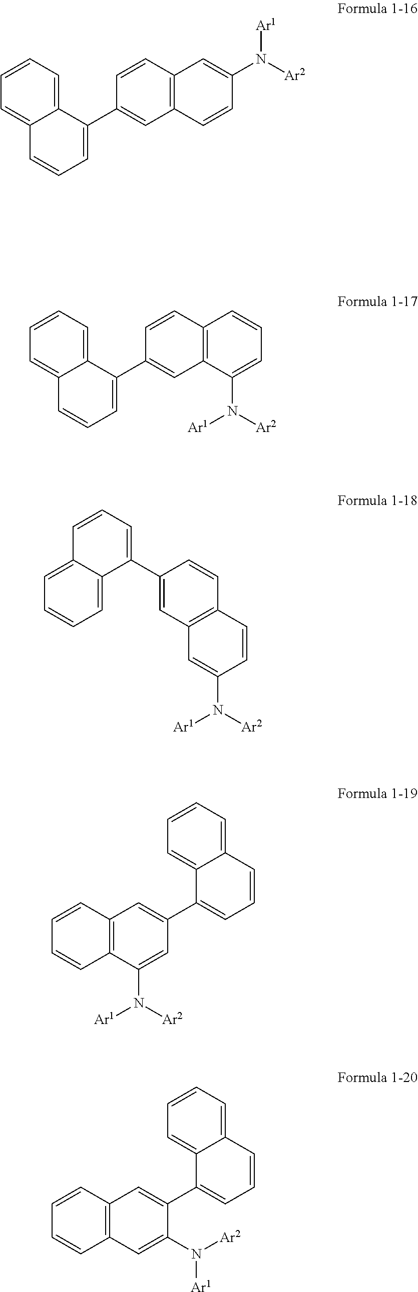 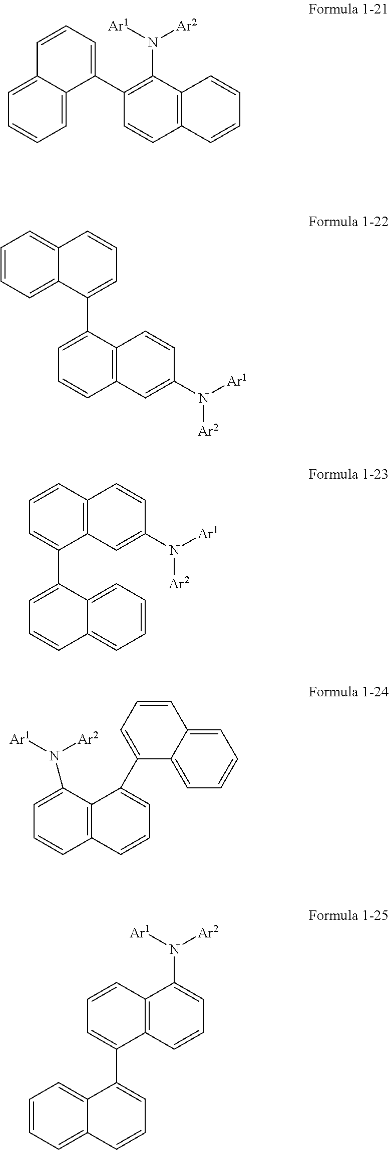 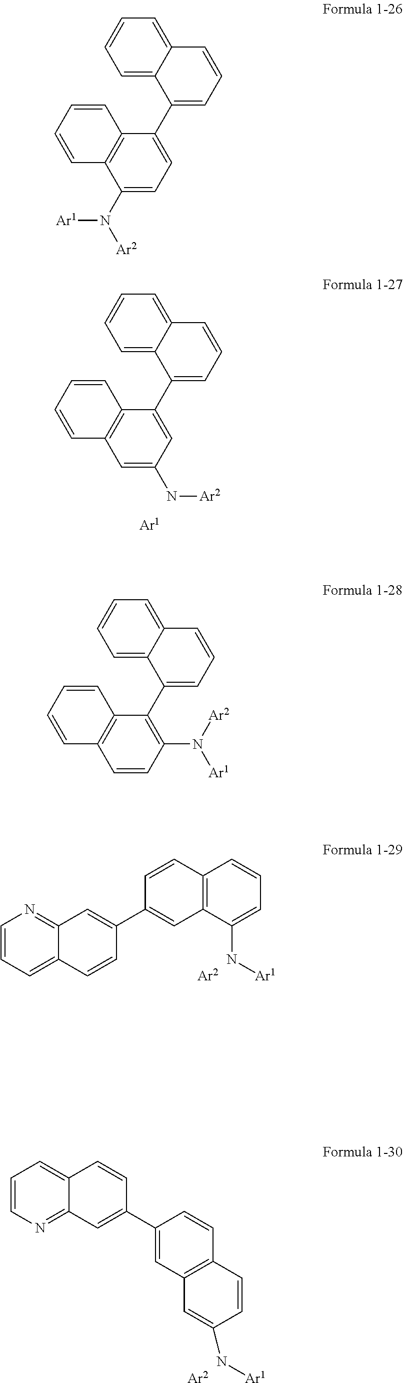 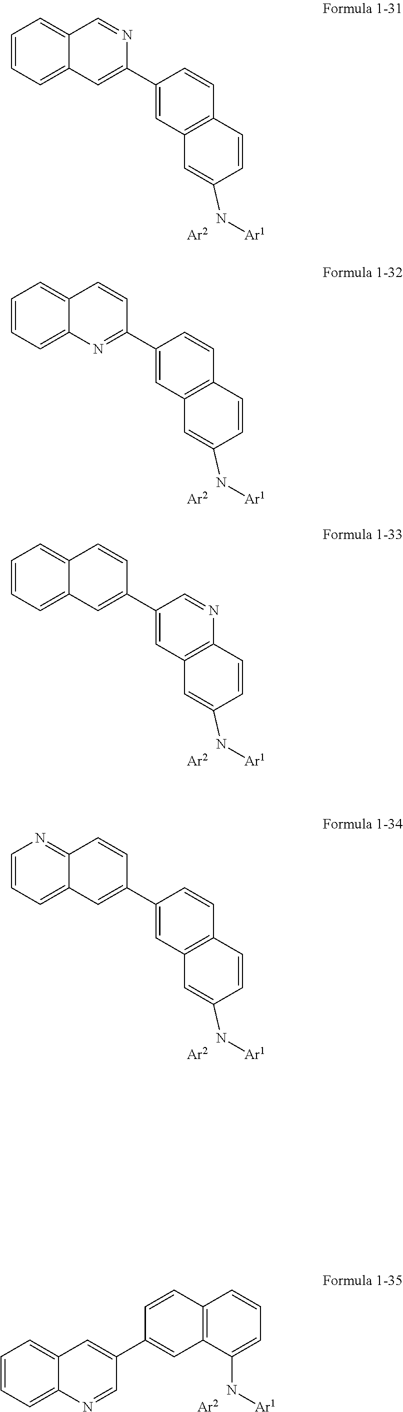 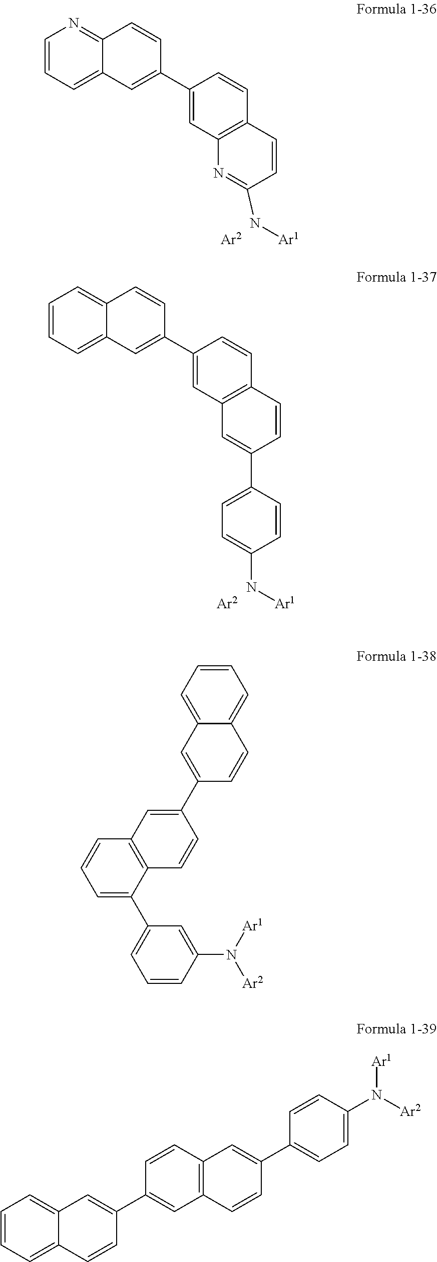 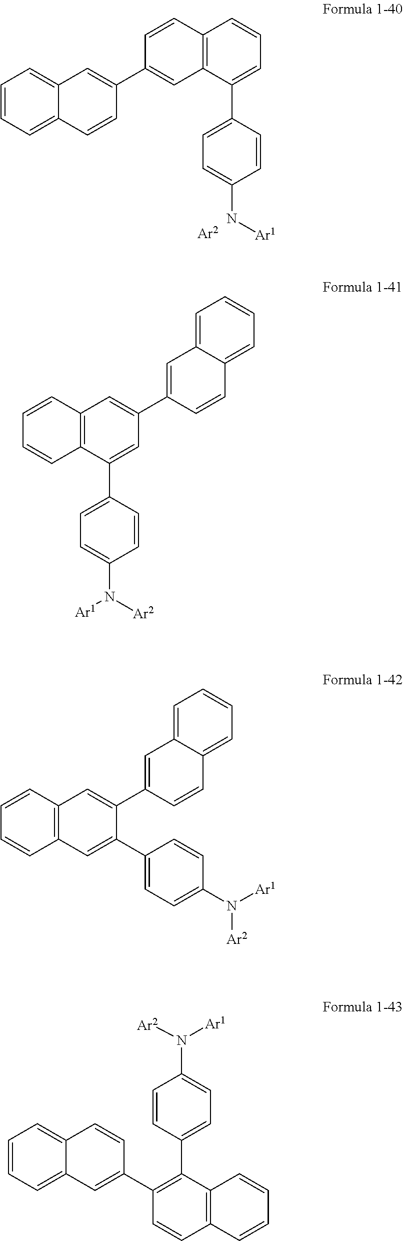 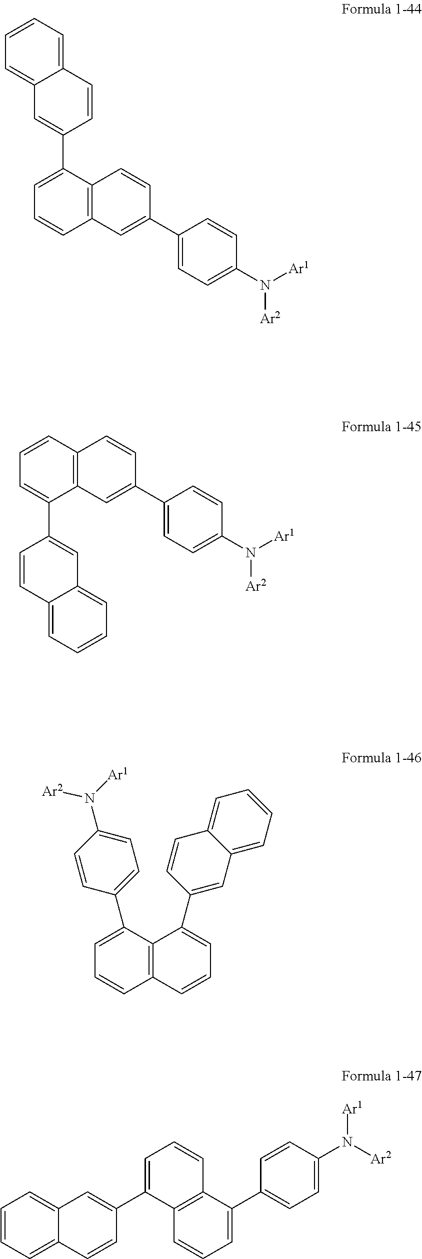 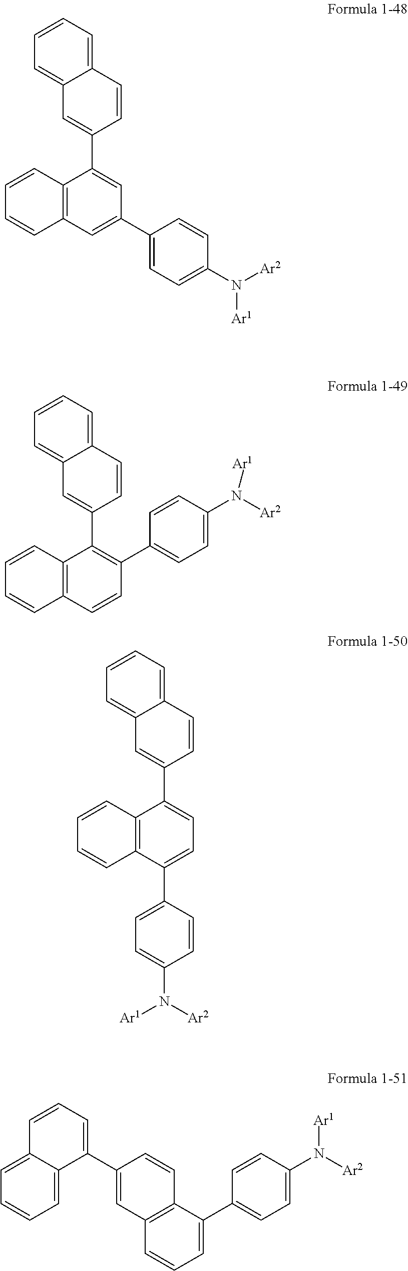 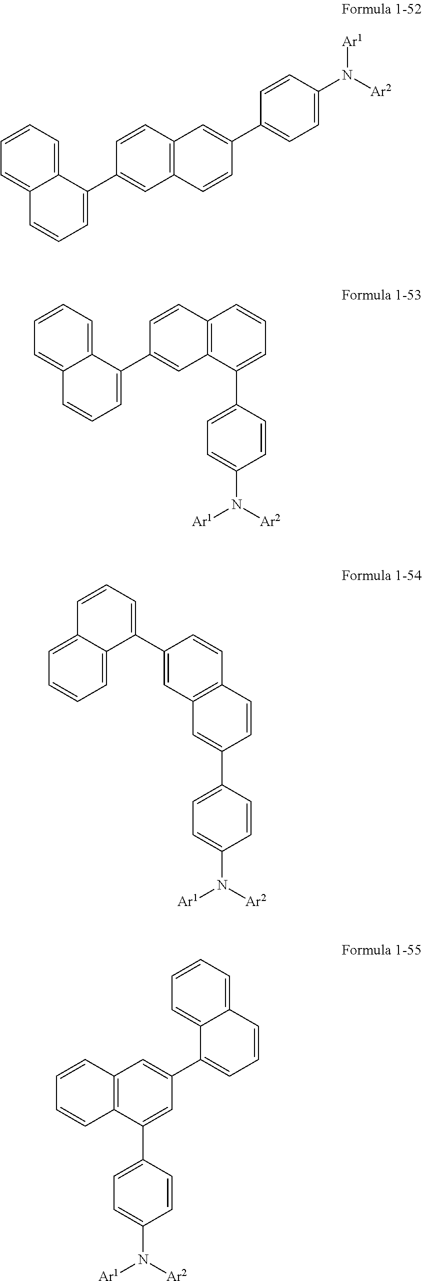 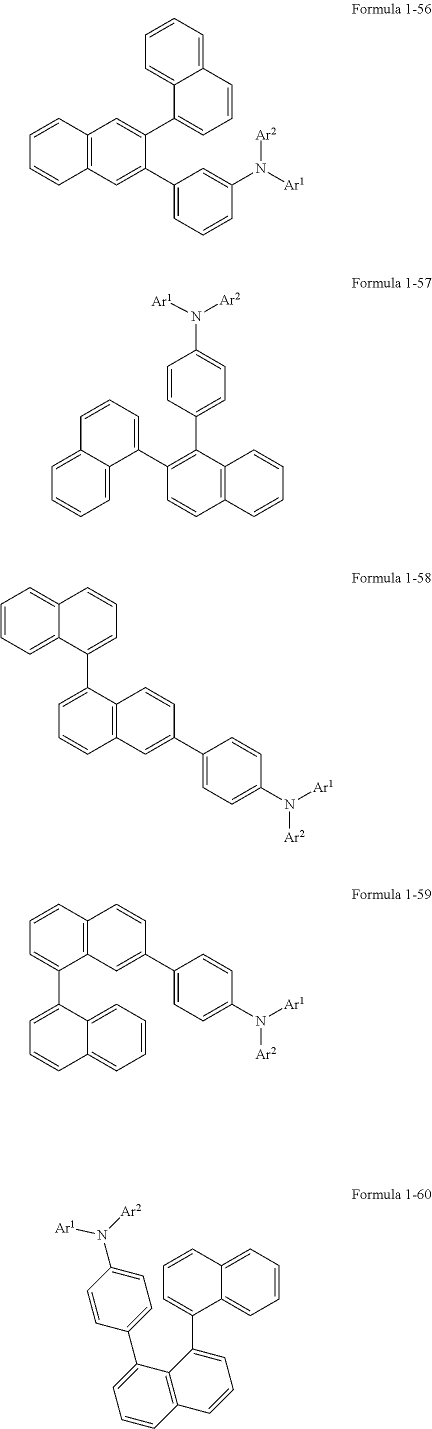 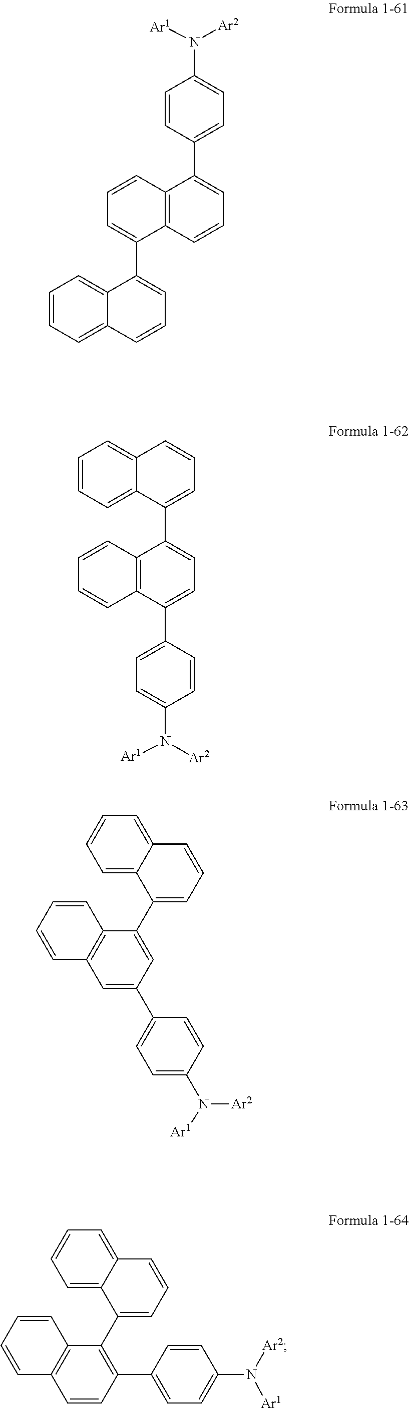
wherein Ar¹-Ar² are defined the same as that in claim 1.

5. The organic electroluminescent compound according to claim 1, wherein in Formula 1-A, Ar² is selected from the following groups: 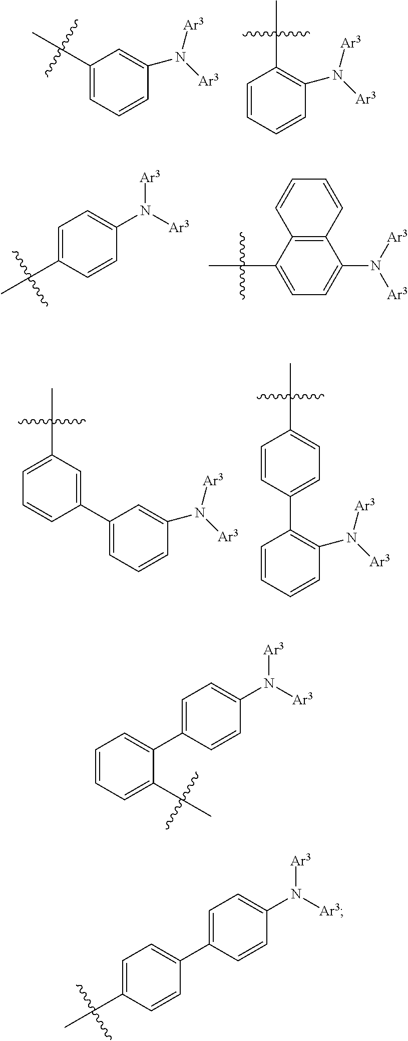
wherein, Ar³ is each independently selected from a substituted or unsubstituted B group, and the B group comprises: phenyl, naphthyl, biphenyl, triphenyl, phenanthryl, fluoranthenyl, triphenylenyl, fluorenyl, dimethylfluorenyl, diphenylfluorenyl, spirobifluorenyl, benzodimethylfluorenyl, benzodiphenylfluorenyl, benzospirobifluorenyl, benzofuryl, dibenzofuryl, naphthobenzofuryl, benzothienyl, dibenzothienyl, naphthobenzothienyl, carbazolyl, phenylcarbazolyl, benzophenylcarbazolyl, and dibenzophenylcarbazolyl.

6. The organic electroluminescent compound according to claim 1, wherein the organic electroluminescent compound has a structure as shown in any one of Formula 1-a to Formula 1-h:

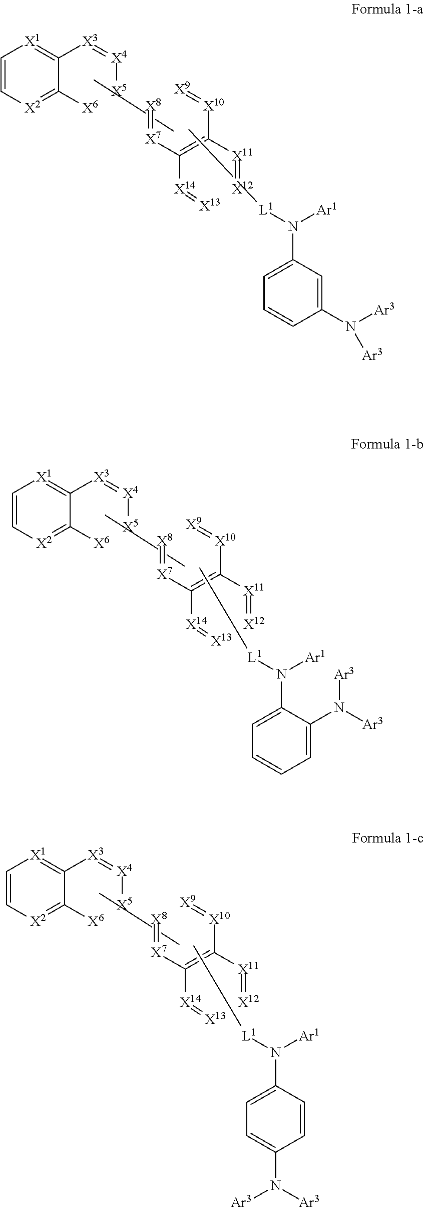

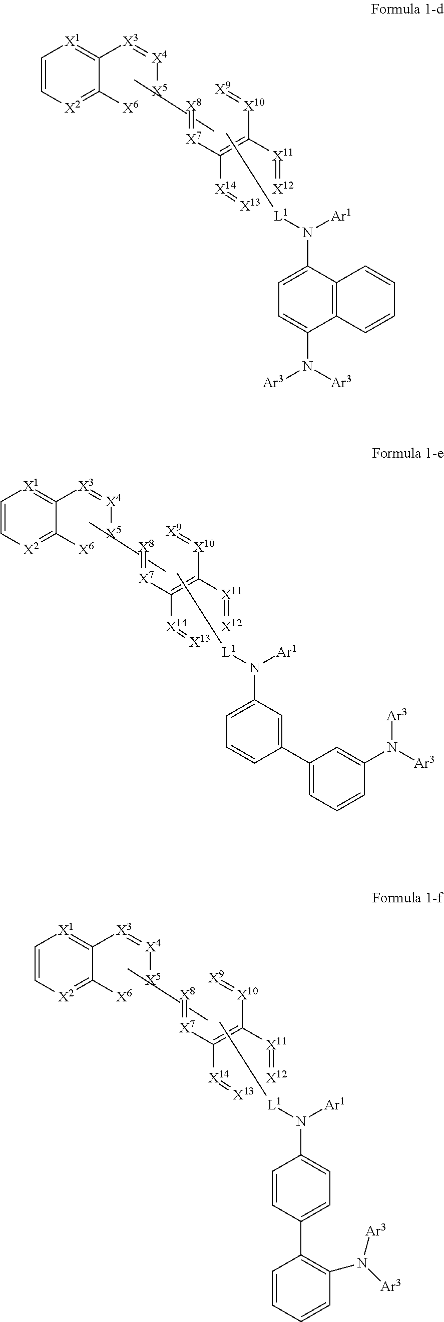

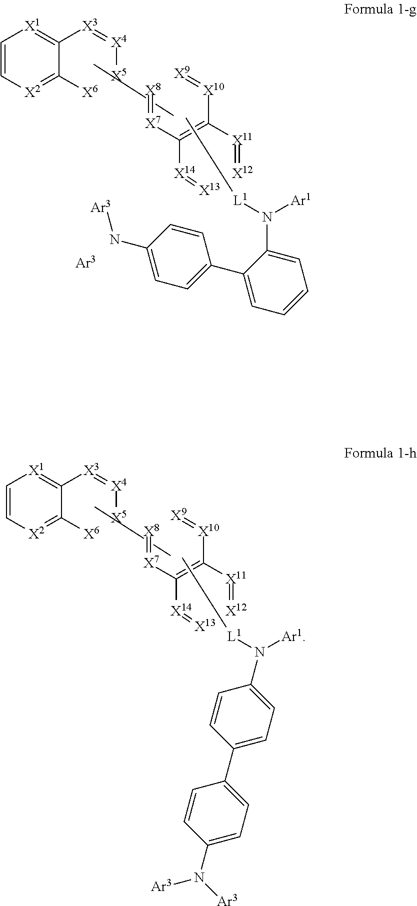

7. The organic electroluminescent compound according to claim 1, wherein the organic electroluminescent compound has a structure as shown in any one of Formula 1-i or Formula 1-ii: 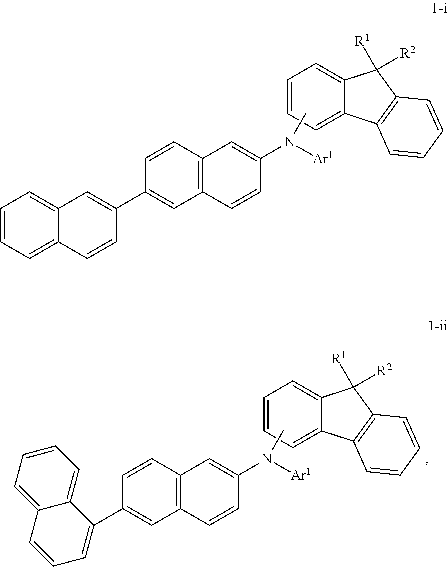
wherein, Ar¹ is defined the same as that in claim 1; R¹-R² are each independently selected from one of or a combination of two of hydrogen, deuterium, a substituted or unsubstituted C1-C6 alkyl, a substituted or unsubstituted C3-C30 cycloalkyl, a substituted or unsubstituted C6-C30 aryl, and a substituted or unsubstituted C3-C30 heteroaryl;
the substituent in the substituted C1-C6 alkyl, the substituted C3-C30 cycloalkyl, the substituted C6-C30 aryl, and the substituted C3-C30 heteroaryl is selected from one of or a combination of two of deuterium, halogen, cyano, a C1-C6 alkyl, a C3-C30 cycloalkyl, a C6-C30 aryl, a C3-C30 heteroaryl, a C6-C60 arylamine group, and a C3-C60 heteroarylamine group.

8. The organic electroluminescent compound according to claim 1, wherein in Formula 1-i or Formula 1-ii, R¹-R² are each independently selected from a C3-C15 cycloalkyl and a C6-C15 aryl;
preferably, R¹ and R² are each independently selected from methyl, phenyl, and fluorenyl;
preferably, Ar¹ is selected from a substituted or unsubstituted C6-C15 aryl and a substituted or unsubstituted C3-C15 heteroaryl; wherein, the substituent of the substituted C6-C15 aryl and the substituted C3-C15 heteroaryl is selected from deuterium, a C1-C3 alkyl, a C3-C10 cycloalkyl, and a C6-C12 aryl;
preferably, Ar¹ is selected from tolyl, phenyl, biphenyl, and dibenzofuryl.

9. The organic electroluminescent compound according to claim 1, wherein the organic electroluminescent compound has a structure as shown in any one of Formula N-i-1 to Formula N-i-72:

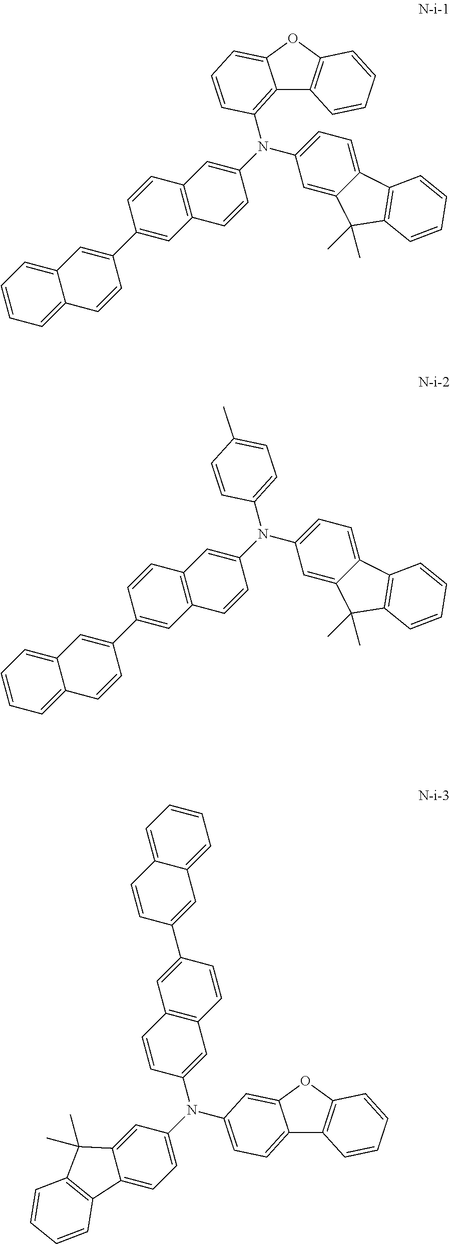 [SMILES: CC1(C)c2ccccc2-c2ccc(N(c3ccc4cc(-c5ccc6ccccc6c5)ccc4c3)c3cccc4oc5ccccc5c34)cc21 ; Cc1ccc(N(c2ccc3c(c2)C(C)(C)c2ccccc2-3)c2ccc3cc(-c4ccc5ccccc5c4)ccc3c2)cc1 ; CC1(C)c2ccccc2-c2ccc(N(c3ccc4cc(-c5ccc6ccccc6c5)ccc4c3)c3ccc4c(c3)oc3ccccc34)cc21]

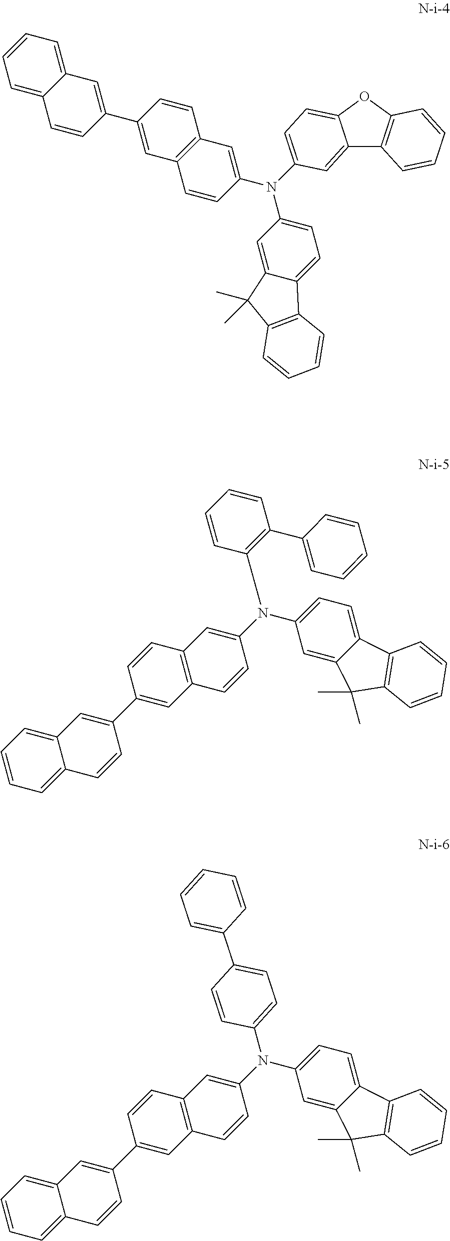 [SMILES: CC1(C)c2ccccc2-c2ccc(N(c3ccc4cc(-c5ccc6ccccc6c5)ccc4c3)c3ccc4oc5ccccc5c4c3)cc21 ; CC1(C)c2ccccc2-c2ccc(N(c3ccc4cc(-c5ccc6ccccc6c5)ccc4c3)c3ccccc3-c3ccccc3)cc21 ; CC1(C)c2ccccc2-c2ccc(N(c3ccc(-c4ccccc4)cc3)c3ccc4cc(-c5ccc6ccccc6c5)ccc4c3)cc21]

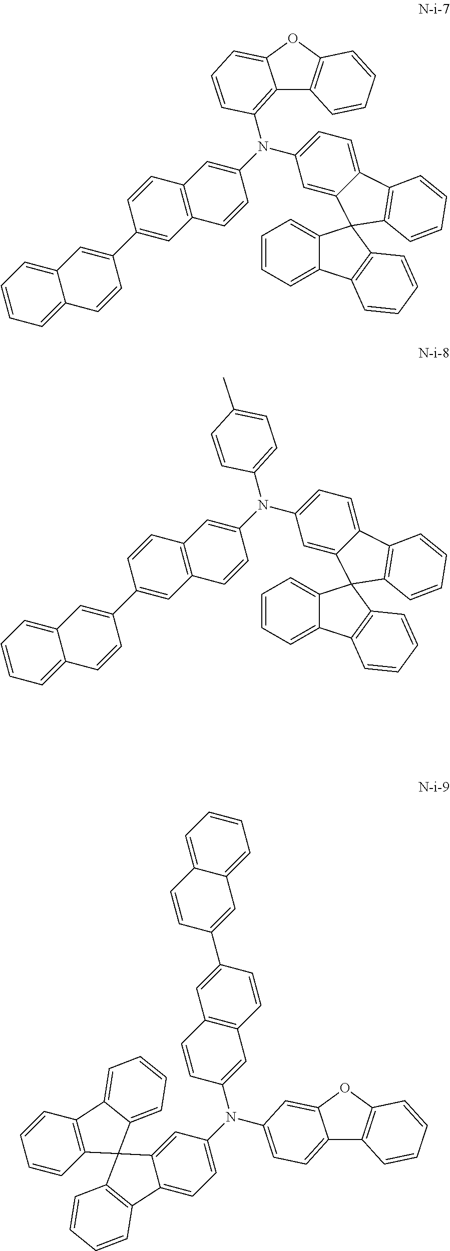 [SMILES: c1ccc2c(c1)-c1ccccc1C21c2ccccc2-c2ccc(N(c3ccc4cc(-c5ccc6ccccc6c5)ccc4c3)c3cccc4oc5ccccc5c34)cc21 ; Cc1ccc(N(c2ccc3c(c2)C2(c4ccccc4-c4ccccc42)c2ccccc2-3)c2ccc3cc(-c4ccc5ccccc5c4)ccc3c2)cc1 ; c1ccc2c(c1)-c1ccccc1C21c2ccccc2-c2ccc(N(c3ccc4cc(-c5ccc6ccccc6c5)ccc4c3)c3ccc4c(c3)oc3ccccc34)cc21]

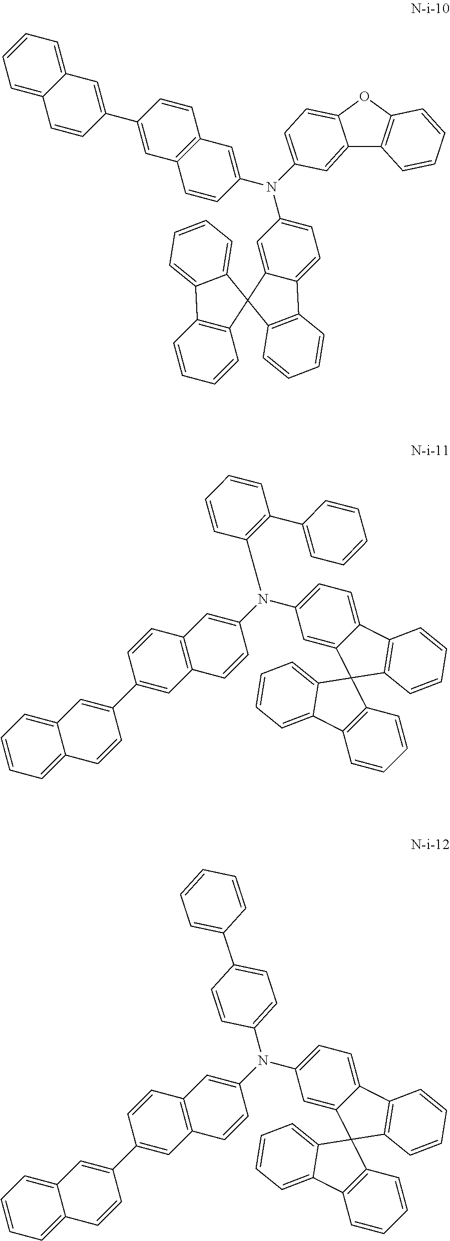 [SMILES: c1ccc2c(c1)-c1ccccc1C21c2ccccc2-c2ccc(N(c3ccc4cc(-c5ccc6ccccc6c5)ccc4c3)c3ccc4oc5ccccc5c4c3)cc21 ; c1ccc(-c2ccccc2N(c2ccc3c(c2)C2(c4ccccc4-c4ccccc42)c2ccccc2-3)c2ccc3cc(-c4ccc5ccccc5c4)ccc3c2)cc1 ; c1ccc(-c2ccc(N(c3ccc4c(c3)C3(c5ccccc5-c5ccccc53)c3ccccc3-4)c3ccc4cc(-c5ccc6ccccc6c5)ccc4c3)cc2)cc1]

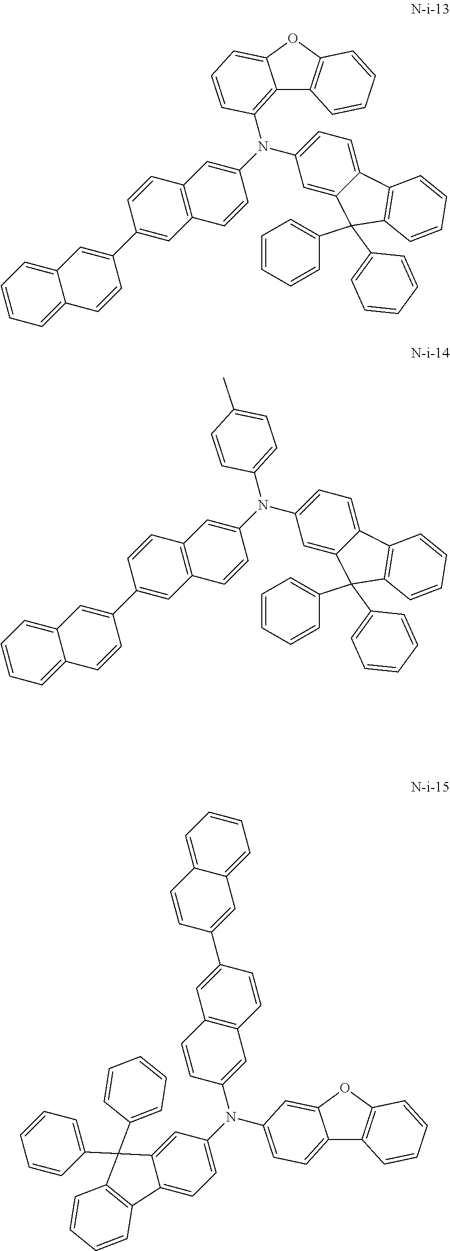 [SMILES: c1ccc(C2(c3ccccc3)c3ccccc3-c3ccc(N(c4ccc5cc(-c6ccc7ccccc7c6)ccc5c4)c4cccc5oc6ccccc6c45)cc32)cc1 ; Cc1ccc(N(c2ccc3c(c2)C(c2ccccc2)(c2ccccc2)c2ccccc2-3)c2ccc3cc(-c4ccc5ccccc5c4)ccc3c2)cc1 ; c1ccc(C2(c3ccccc3)c3ccccc3-c3ccc(N(c4ccc5cc(-c6ccc7ccccc7c6)ccc5c4)c4ccc5c(c4)oc4ccccc45)cc32)cc1]

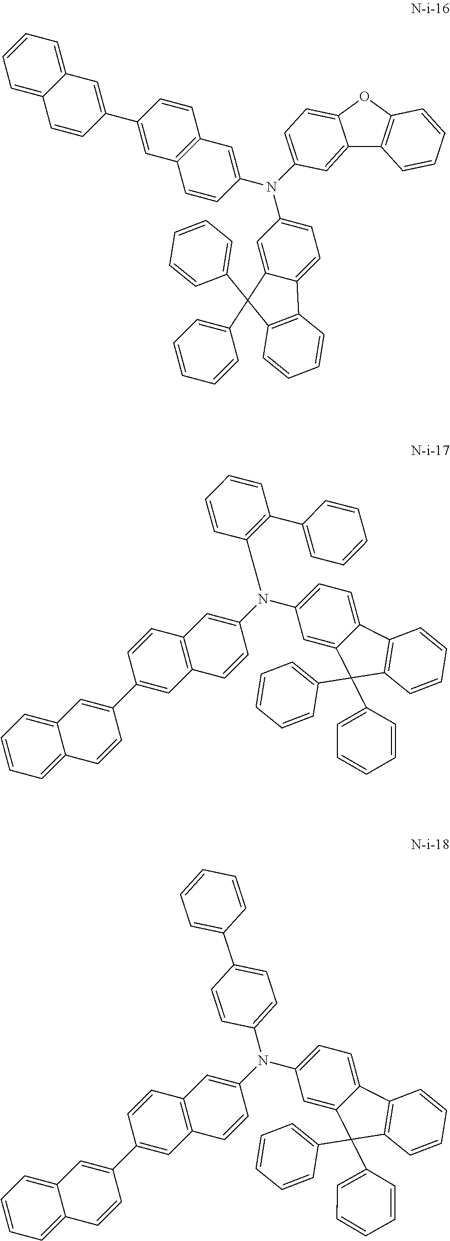 [SMILES: c1ccc(C2(c3ccccc3)c3ccccc3-c3ccc(N(c4ccc5cc(-c6ccc7ccccc7c6)ccc5c4)c4ccc5oc6ccccc6c5c4)cc32)cc1 ; c1ccc(-c2ccccc2N(c2ccc3c(c2)C(c2ccccc2)(c2ccccc2)c2ccccc2-3)c2ccc3cc(-c4ccc5ccccc5c4)ccc3c2)cc1 ; c1ccc(-c2ccc(N(c3ccc4c(c3)C(c3ccccc3)(c3ccccc3)c3ccccc3-4)c3ccc4cc(-c5ccc6ccccc6c5)ccc4c3)cc2)cc1]

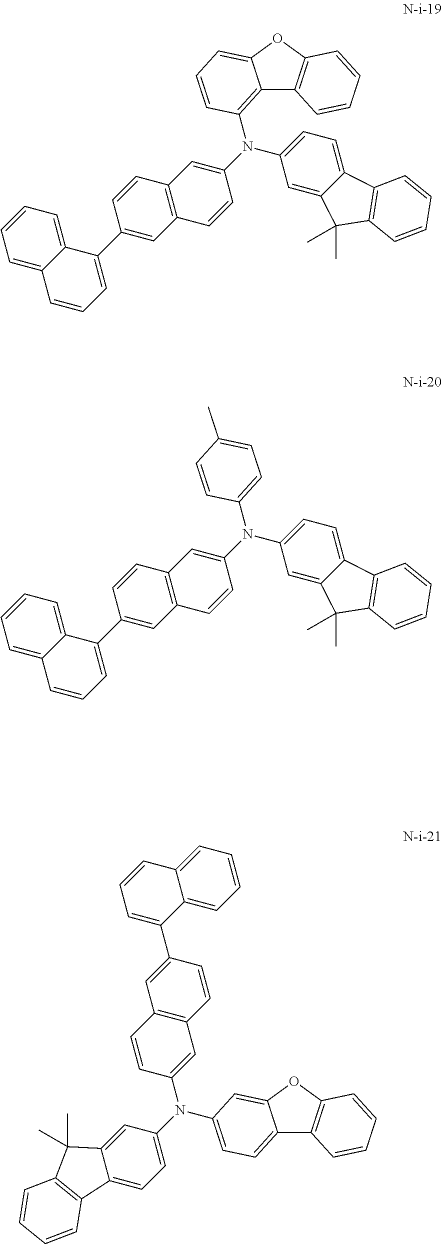 [SMILES: CC1(C)c2ccccc2-c2ccc(N(c3ccc4cc(-c5cccc6ccccc56)ccc4c3)c3cccc4oc5ccccc5c34)cc21 ; Cc1ccc(N(c2ccc3c(c2)C(C)(C)c2ccccc2-3)c2ccc3cc(-c4cccc5ccccc45)ccc3c2)cc1 ; CC1(C)c2ccccc2-c2ccc(N(c3ccc4cc(-c5cccc6ccccc56)ccc4c3)c3ccc4c(c3)oc3ccccc34)cc21]

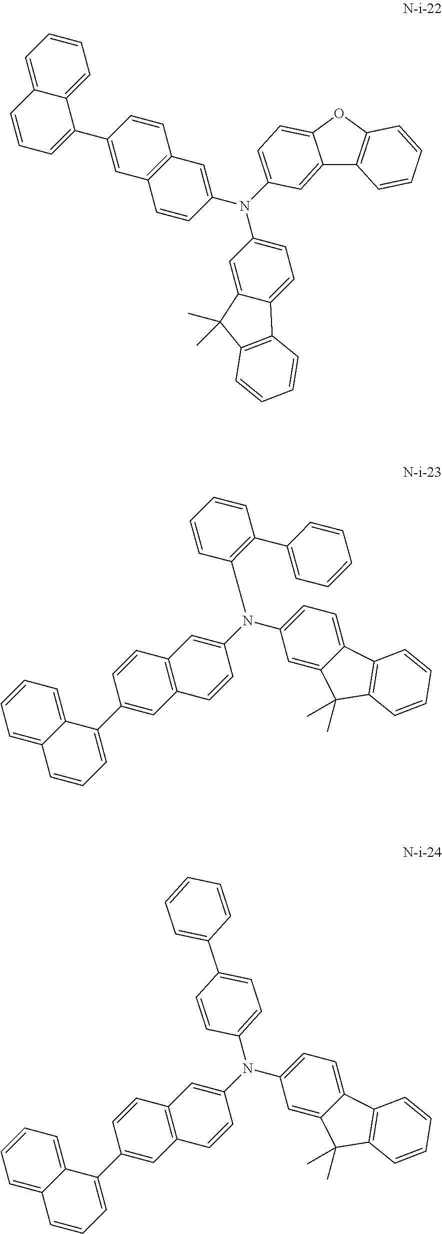 [SMILES: CC1(C)c2ccccc2-c2ccc(N(c3ccc4cc(-c5cccc6ccccc56)ccc4c3)c3ccc4oc5ccccc5c4c3)cc21 ; CC1(C)c2ccccc2-c2ccc(N(c3ccc4cc(-c5cccc6ccccc56)ccc4c3)c3ccccc3-c3ccccc3)cc21 ; CC1(C)c2ccccc2-c2ccc(N(c3ccc(-c4ccccc4)cc3)c3ccc4cc(-c5cccc6ccccc56)ccc4c3)cc21]

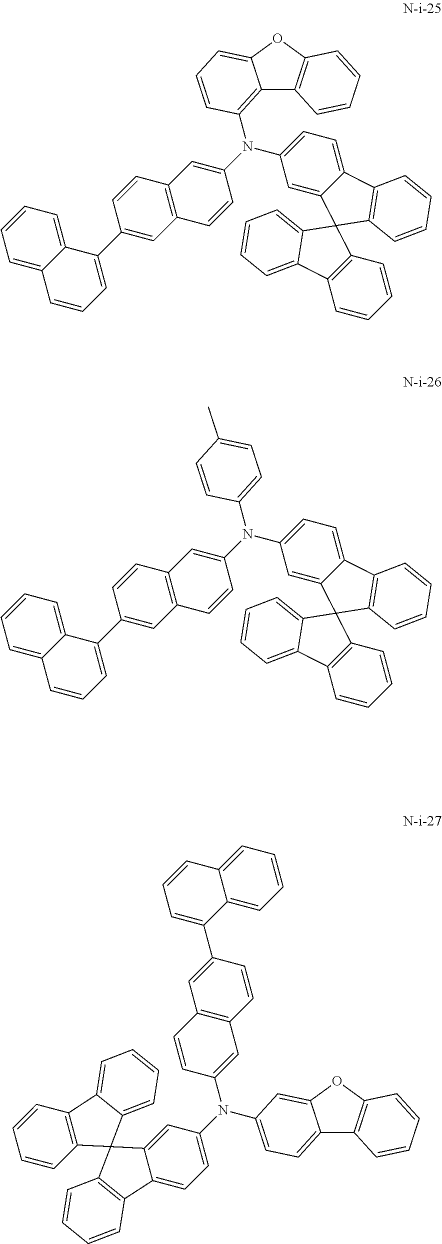 [SMILES: c1ccc2c(c1)-c1ccccc1C21c2ccccc2-c2ccc(N(c3ccc4cc(-c5cccc6ccccc56)ccc4c3)c3cccc4oc5ccccc5c34)cc21 ; Cc1ccc(N(c2ccc3c(c2)C2(c4ccccc4-c4ccccc42)c2ccccc2-3)c2ccc3cc(-c4cccc5ccccc45)ccc3c2)cc1 ; c1ccc2c(c1)-c1ccccc1C21c2ccccc2-c2ccc(N(c3ccc4cc(-c5cccc6ccccc56)ccc4c3)c3ccc4c(c3)oc3ccccc34)cc21]

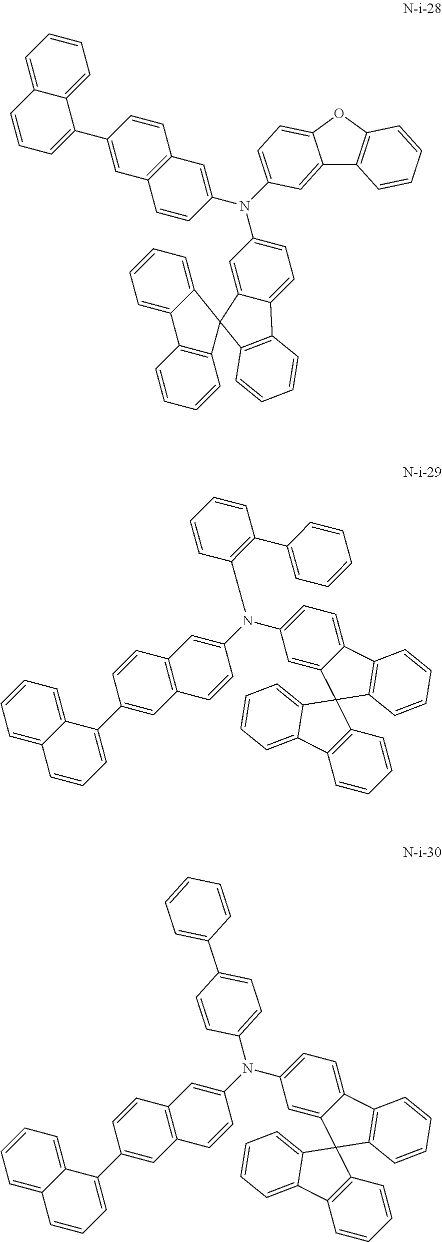 [SMILES: c1ccc2c(c1)-c1ccccc1C21c2ccccc2-c2ccc(N(c3ccc4cc(-c5cccc6ccccc56)ccc4c3)c3ccc4oc5ccccc5c4c3)cc21 ; c1ccc(-c2ccccc2N(c2ccc3c(c2)C2(c4ccccc4-c4ccccc42)c2ccccc2-3)c2ccc3cc(-c4cccc5ccccc45)ccc3c2)cc1 ; c1ccc(-c2ccc(N(c3ccc4c(c3)C3(c5ccccc5-c5ccccc53)c3ccccc3-4)c3ccc4cc(-c5cccc6ccccc56)ccc4c3)cc2)cc1]

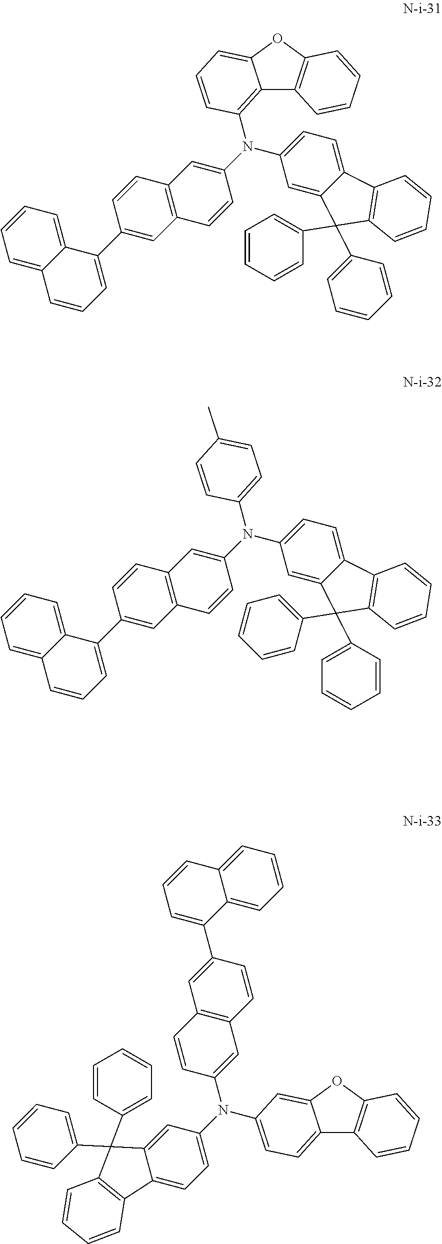 [SMILES: c1ccc(C2(c3ccccc3)c3ccccc3-c3ccc(N(c4ccc5cc(-c6cccc7ccccc67)ccc5c4)c4cccc5oc6ccccc6c45)cc32)cc1 ; Cc1ccc(N(c2ccc3c(c2)C(c2ccccc2)(c2ccccc2)c2ccccc2-3)c2ccc3cc(-c4cccc5ccccc45)ccc3c2)cc1 ; c1ccc(C2(c3ccccc3)c3ccccc3-c3ccc(N(c4ccc5cc(-c6cccc7ccccc67)ccc5c4)c4ccc5c(c4)oc4ccccc45)cc32)cc1]

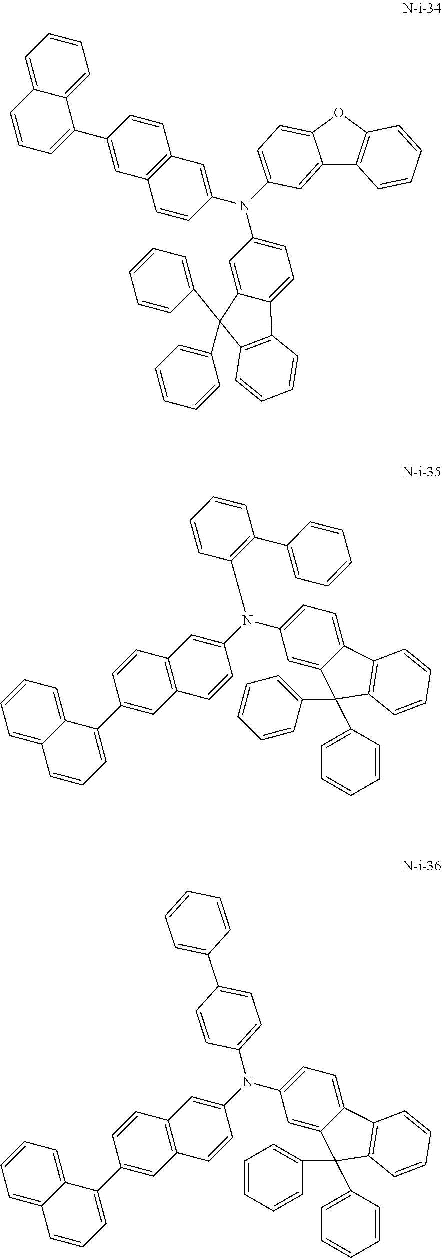 [SMILES: c1ccc(C2(c3ccccc3)c3ccccc3-c3ccc(N(c4ccc5cc(-c6cccc7ccccc67)ccc5c4)c4ccc5oc6ccccc6c5c4)cc32)cc1 ; c1ccc(-c2ccccc2N(c2ccc3c(c2)C(c2ccccc2)(c2ccccc2)c2ccccc2-3)c2ccc3cc(-c4cccc5ccccc45)ccc3c2)cc1 ; c1ccc(-c2ccc(N(c3ccc4c(c3)C(c3ccccc3)(c3ccccc3)c3ccccc3-4)c3ccc4cc(-c5cccc6ccccc56)ccc4c3)cc2)cc1]

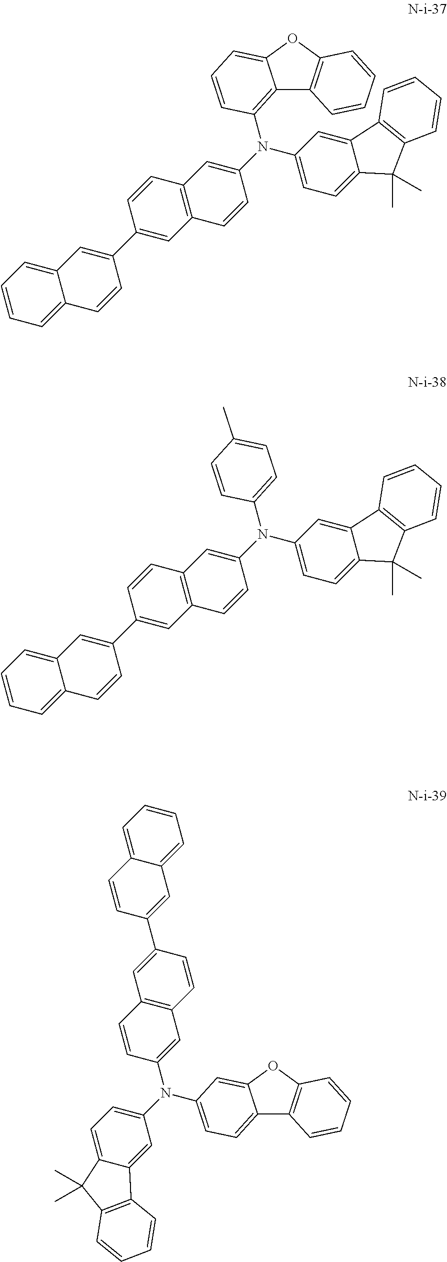 [SMILES: CC1(C)c2ccccc2-c2cc(N(c3ccc4cc(-c5ccc6ccccc6c5)ccc4c3)c3cccc4oc5ccccc5c34)ccc21 ; Cc1ccc(N(c2ccc3c(c2)-c2ccccc2C3(C)C)c2ccc3cc(-c4ccc5ccccc5c4)ccc3c2)cc1 ; CC1(C)c2ccccc2-c2cc(N(c3ccc4cc(-c5ccc6ccccc6c5)ccc4c3)c3ccc4c(c3)oc3ccccc34)ccc21]

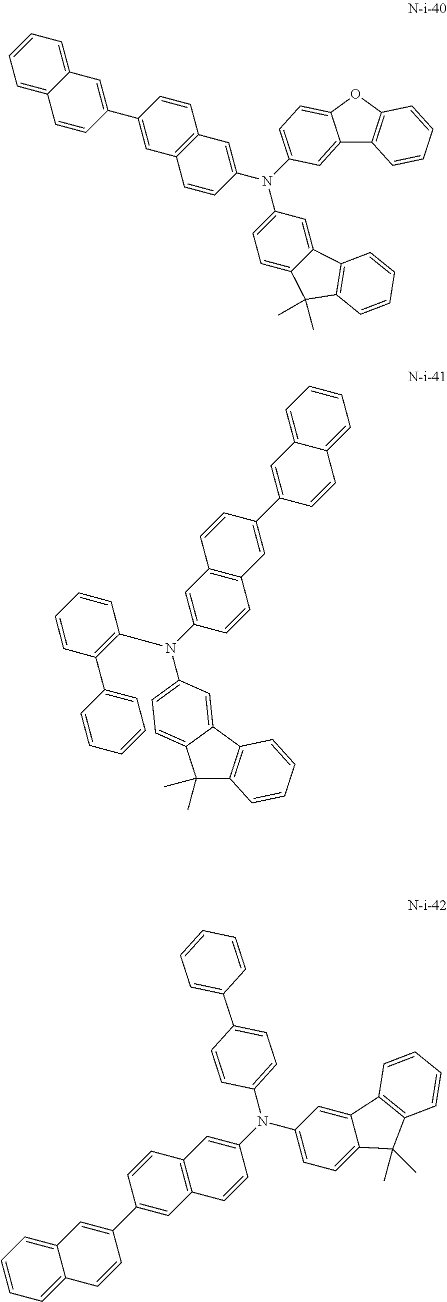 [SMILES: CC1(C)c2ccccc2-c2cc(N(c3ccc4cc(-c5ccc6ccccc6c5)ccc4c3)c3ccc4oc5ccccc5c4c3)ccc21 ; CC1(C)c2ccccc2-c2cc(N(c3ccc4cc(-c5ccc6ccccc6c5)ccc4c3)c3ccccc3-c3ccccc3)ccc21 ; CC1(C)c2ccccc2-c2cc(N(c3ccc(-c4ccccc4)cc3)c3ccc4cc(-c5ccc6ccccc6c5)ccc4c3)ccc21]

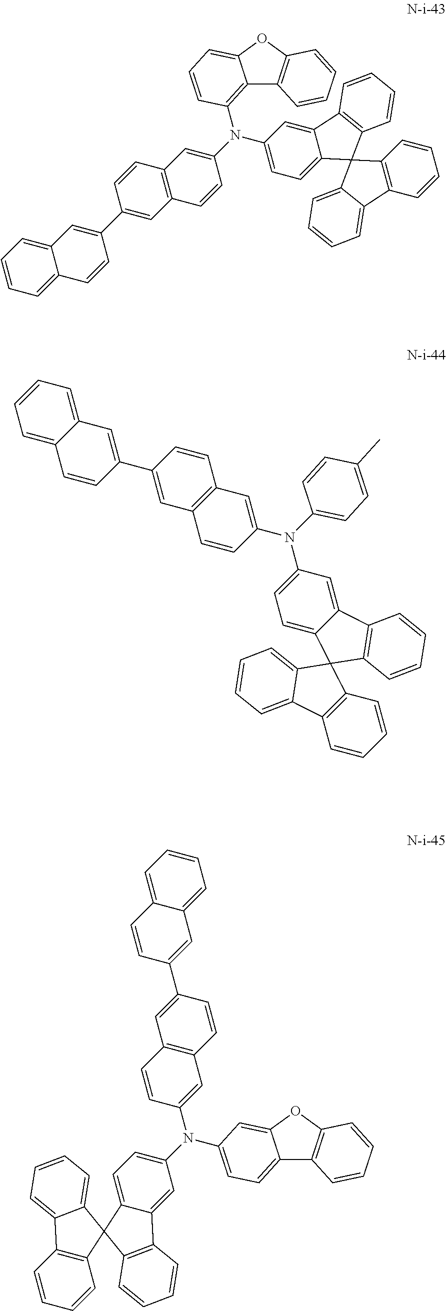 [SMILES: c1ccc2c(c1)-c1ccccc1C21c2ccccc2-c2cc(N(c3ccc4cc(-c5ccc6ccccc6c5)ccc4c3)c3cccc4oc5ccccc5c34)ccc21 ; Cc1ccc(N(c2ccc3c(c2)-c2ccccc2C32c3ccccc3-c3ccccc32)c2ccc3cc(-c4ccc5ccccc5c4)ccc3c2)cc1 ; c1ccc2c(c1)-c1ccccc1C21c2ccccc2-c2cc(N(c3ccc4cc(-c5ccc6ccccc6c5)ccc4c3)c3ccc4c(c3)oc3ccccc34)ccc21]

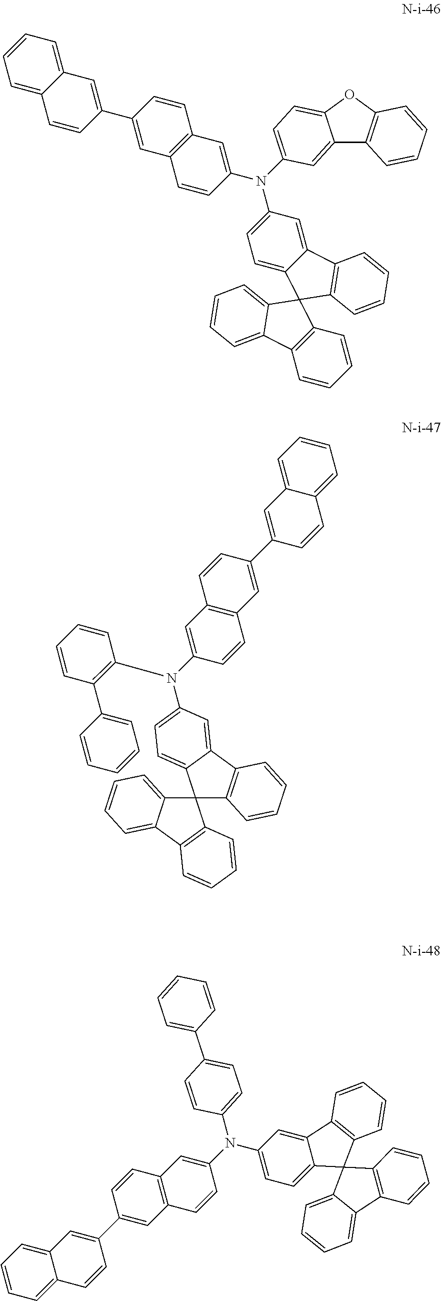 [SMILES: c1ccc2c(c1)-c1ccccc1C21c2ccccc2-c2cc(N(c3ccc4cc(-c5ccc6ccccc6c5)ccc4c3)c3ccc4oc5ccccc5c4c3)ccc21 ; c1ccc(-c2ccccc2N(c2ccc3c(c2)-c2ccccc2C32c3ccccc3-c3ccccc32)c2ccc3cc(-c4ccc5ccccc5c4)ccc3c2)cc1 ; c1ccc(-c2ccc(N(c3ccc4c(c3)-c3ccccc3C43c4ccccc4-c4ccccc43)c3ccc4cc(-c5ccc6ccccc6c5)ccc4c3)cc2)cc1]

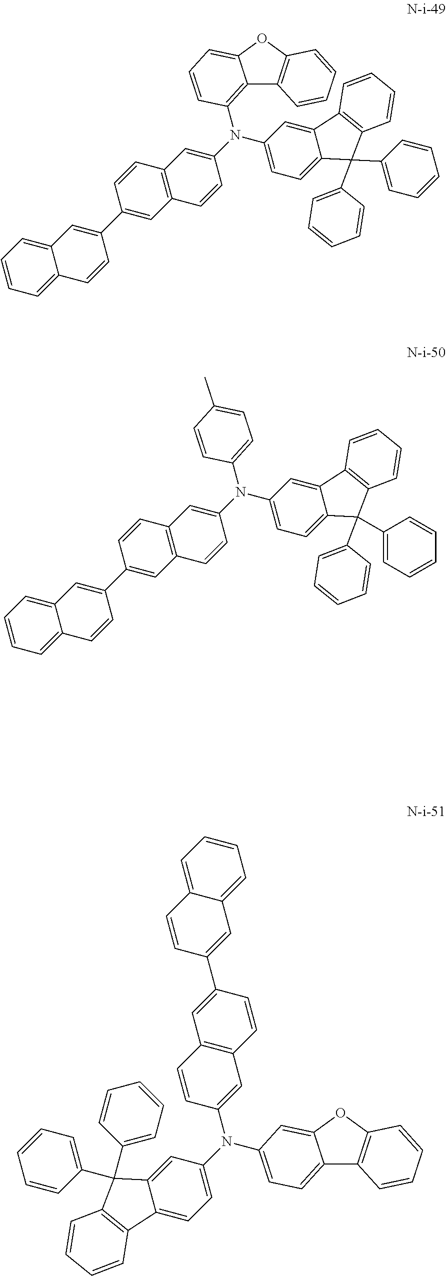 [SMILES: c1ccc(C2(c3ccccc3)c3ccccc3-c3cc(N(c4ccc5cc(-c6ccc7ccccc7c6)ccc5c4)c4cccc5oc6ccccc6c45)ccc32)cc1 ; Cc1ccc(N(c2ccc3c(c2)-c2ccccc2C3(c2ccccc2)c2ccccc2)c2ccc3cc(-c4ccc5ccccc5c4)ccc3c2)cc1 ; c1ccc(C2(c3ccccc3)c3ccccc3-c3ccc(N(c4ccc5cc(-c6ccc7ccccc7c6)ccc5c4)c4ccc5c(c4)oc4ccccc45)cc32)cc1]

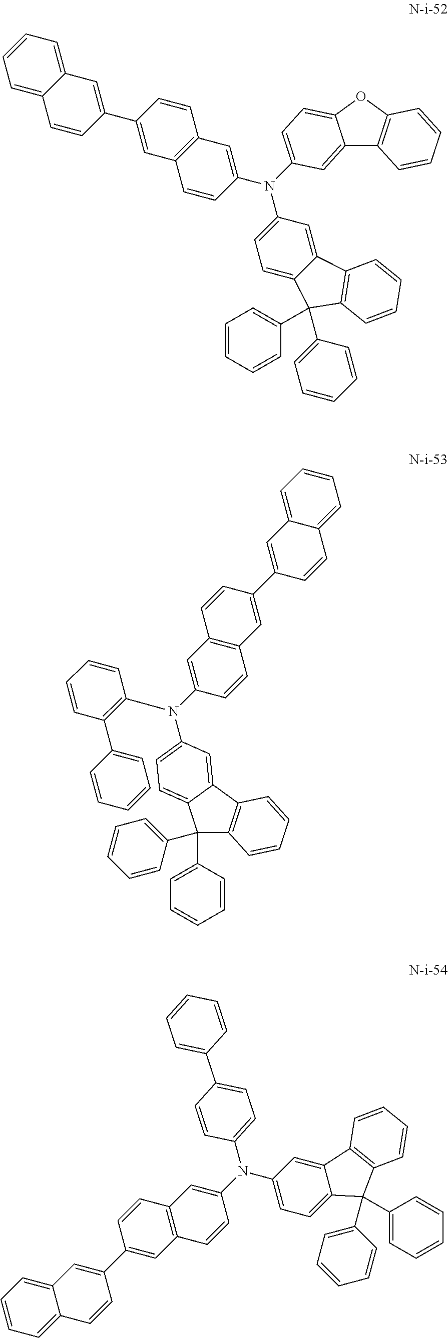 [SMILES: c1ccc(C2(c3ccccc3)c3ccccc3-c3cc(N(c4ccc5cc(-c6ccc7ccccc7c6)ccc5c4)c4ccc5oc6ccccc6c5c4)ccc32)cc1 ; c1ccc(-c2ccccc2N(c2ccc3c(c2)-c2ccccc2C3(c2ccccc2)c2ccccc2)c2ccc3cc(-c4ccc5ccccc5c4)ccc3c2)cc1 ; c1ccc(-c2ccc(N(c3ccc4c(c3)-c3ccccc3C4(c3ccccc3)c3ccccc3)c3ccc4cc(-c5ccc6ccccc6c5)ccc4c3)cc2)cc1]

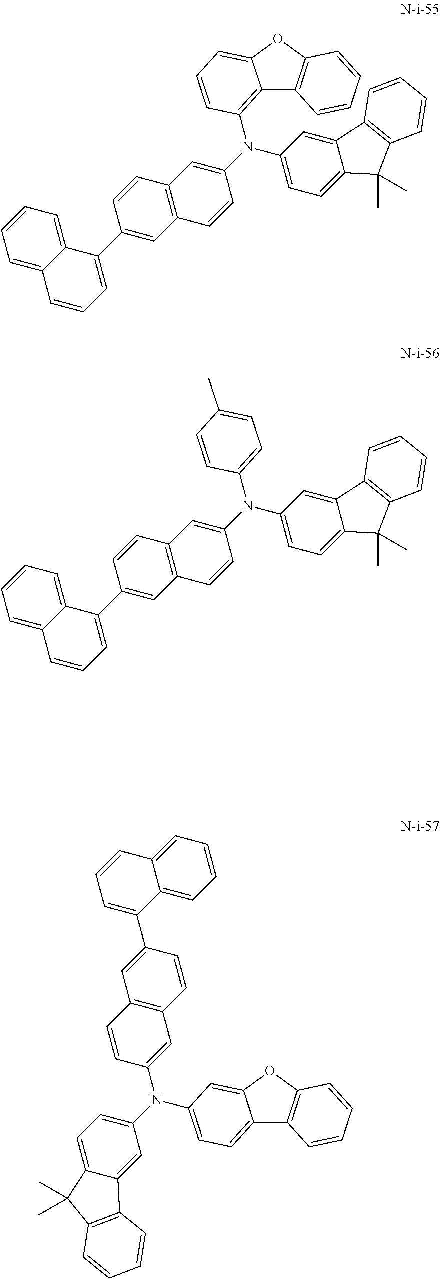 [SMILES: CC1(C)c2ccccc2-c2cc(N(c3ccc4cc(-c5cccc6ccccc56)ccc4c3)c3cccc4oc5ccccc5c34)ccc21 ; Cc1ccc(N(c2ccc3c(c2)-c2ccccc2C3(C)C)c2ccc3cc(-c4cccc5ccccc45)ccc3c2)cc1 ; CC1(C)c2ccccc2-c2cc(N(c3ccc4cc(-c5cccc6ccccc56)ccc4c3)c3ccc4c(c3)oc3ccccc34)ccc21]

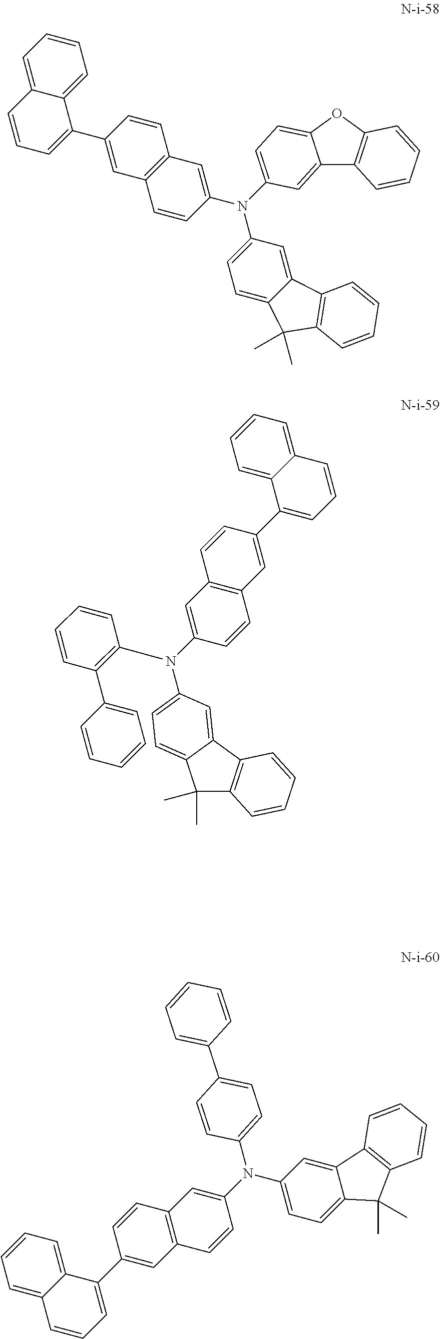 [SMILES: CC1(C)c2ccccc2-c2cc(N(c3ccc4cc(-c5cccc6ccccc56)ccc4c3)c3ccc4oc5ccccc5c4c3)ccc21 ; CC1(C)c2ccccc2-c2cc(N(c3ccc4cc(-c5cccc6ccccc56)ccc4c3)c3ccccc3-c3ccccc3)ccc21 ; CC1(C)c2ccccc2-c2cc(N(c3ccc(-c4ccccc4)cc3)c3ccc4cc(-c5cccc6ccccc56)ccc4c3)ccc21]

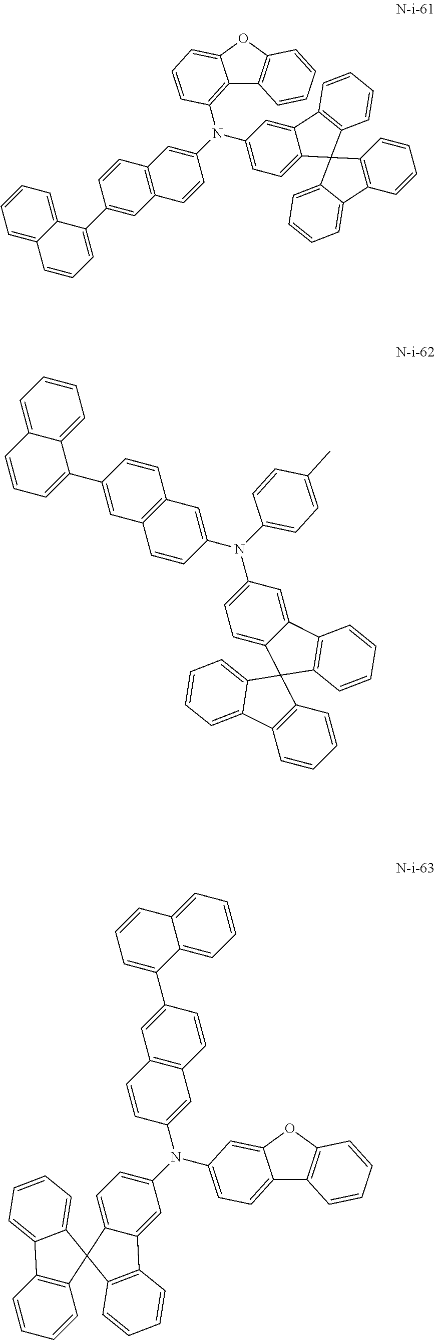 [SMILES: c1ccc2c(c1)-c1ccccc1C21c2ccccc2-c2cc(N(c3ccc4cc(-c5cccc6ccccc56)ccc4c3)c3cccc4oc5ccccc5c34)ccc21 ; Cc1ccc(N(c2ccc3c(c2)-c2ccccc2C32c3ccccc3-c3ccccc32)c2ccc3cc(-c4cccc5ccccc45)ccc3c2)cc1 ; c1ccc2c(c1)-c1ccccc1C21c2ccccc2-c2cc(N(c3ccc4cc(-c5cccc6ccccc56)ccc4c3)c3ccc4c(c3)oc3ccccc34)ccc21]

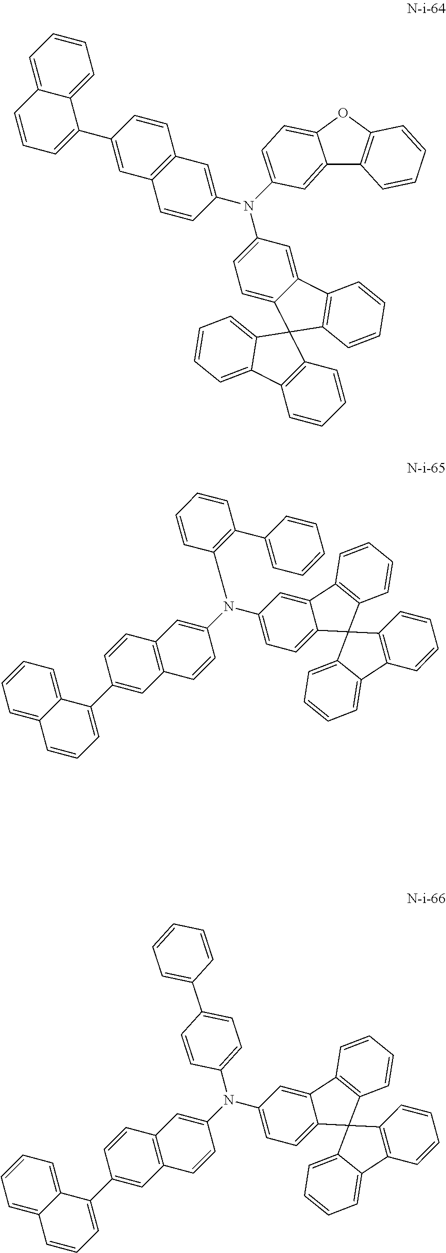 [SMILES: c1ccc2c(c1)-c1ccccc1C21c2ccccc2-c2cc(N(c3ccc4cc(-c5cccc6ccccc56)ccc4c3)c3ccc4oc5ccccc5c4c3)ccc21 ; c1ccc(-c2ccccc2N(c2ccc3c(c2)-c2ccccc2C32c3ccccc3-c3ccccc32)c2ccc3cc(-c4cccc5ccccc45)ccc3c2)cc1 ; c1ccc(-c2ccc(N(c3ccc4c(c3)-c3ccccc3C43c4ccccc4-c4ccccc43)c3ccc4cc(-c5cccc6ccccc56)ccc4c3)cc2)cc1]

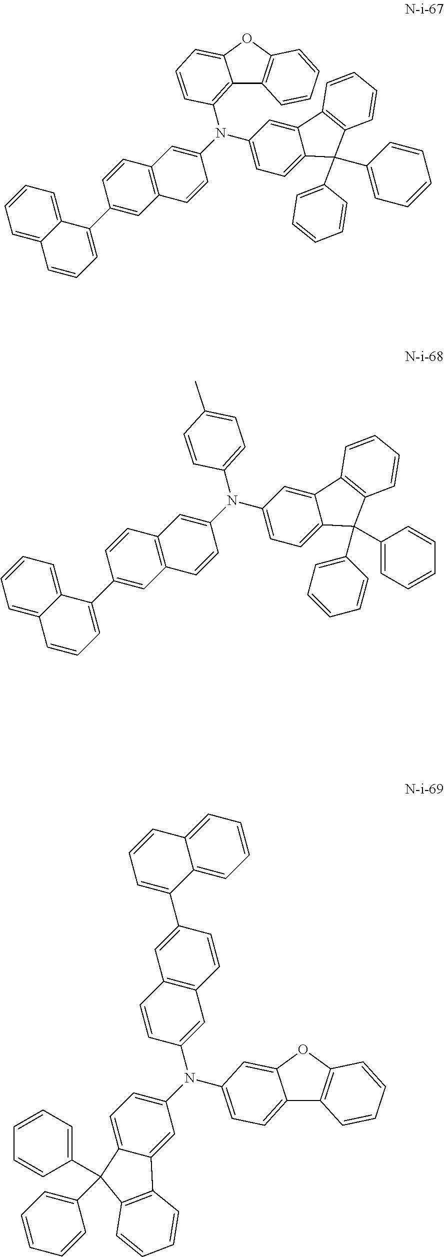 [SMILES: c1ccc(C2(c3ccccc3)c3ccccc3-c3cc(N(c4ccc5cc(-c6cccc7ccccc67)ccc5c4)c4cccc5oc6ccccc6c45)ccc32)cc1 ; Cc1ccc(N(c2ccc3c(c2)-c2ccccc2C3(c2ccccc2)c2ccccc2)c2ccc3cc(-c4cccc5ccccc45)ccc3c2)cc1 ; c1ccc(C2(c3ccccc3)c3ccccc3-c3cc(N(c4ccc5cc(-c6cccc7ccccc67)ccc5c4)c4ccc5c(c4)oc4ccccc45)ccc32)cc1]

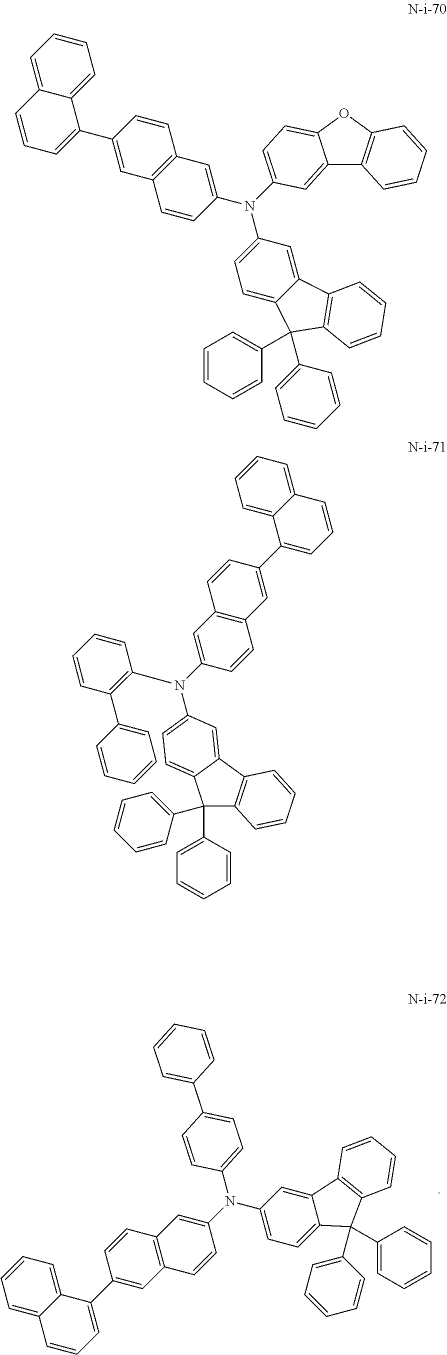 [SMILES: c1ccc(C2(c3ccccc3)c3ccccc3-c3cc(N(c4ccc5cc(-c6cccc7ccccc67)ccc5c4)c4ccc5oc6ccccc6c5c4)ccc32)cc1 ; c1ccc(-c2ccccc2N(c2ccc3c(c2)-c2ccccc2C3(c2ccccc2)c2ccccc2)c2ccc3cc(-c4cccc5ccccc45)ccc3c2)cc1 ; c1ccc(-c2ccc(N(c3ccc4c(c3)-c3ccccc3C4(c3ccccc3)c3ccccc3)c3ccc4cc(-c5cccc6ccccc56)ccc4c3)cc2)cc1]

10. The organic electroluminescent compound according to claim 1, wherein the organic electroluminescent compound has a structure as shown in any one of N-1 to N-549:

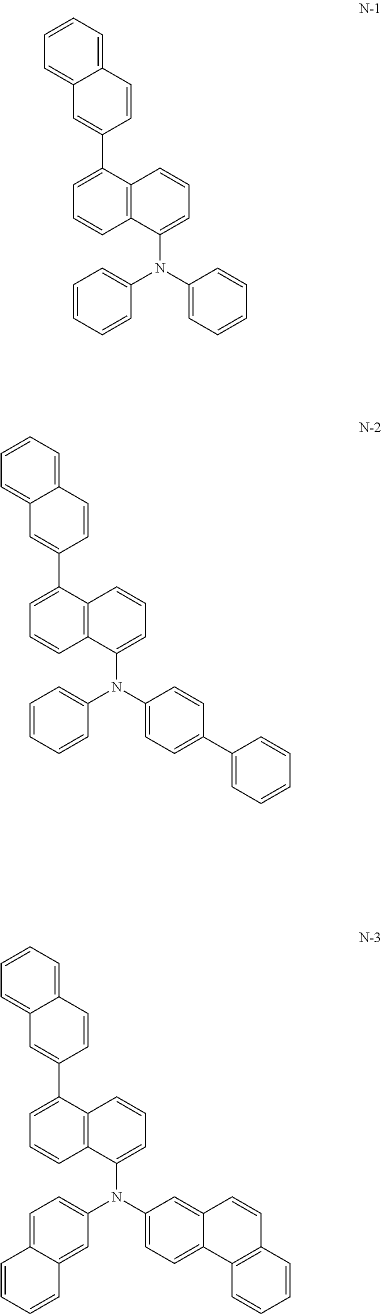 [SMILES: c1ccc(N(c2ccccc2)c2cccc3c(-c4ccc5ccccc5c4)cccc23)cc1 ; c1ccc(-c2ccc(N(c3ccccc3)c3cccc4c(-c5ccc6ccccc6c5)cccc34)cc2)cc1 ; c1ccc2cc(-c3cccc4c(N(c5ccc6ccccc6c5)c5ccc6c(ccc7ccccc76)c5)cccc34)ccc2c1]

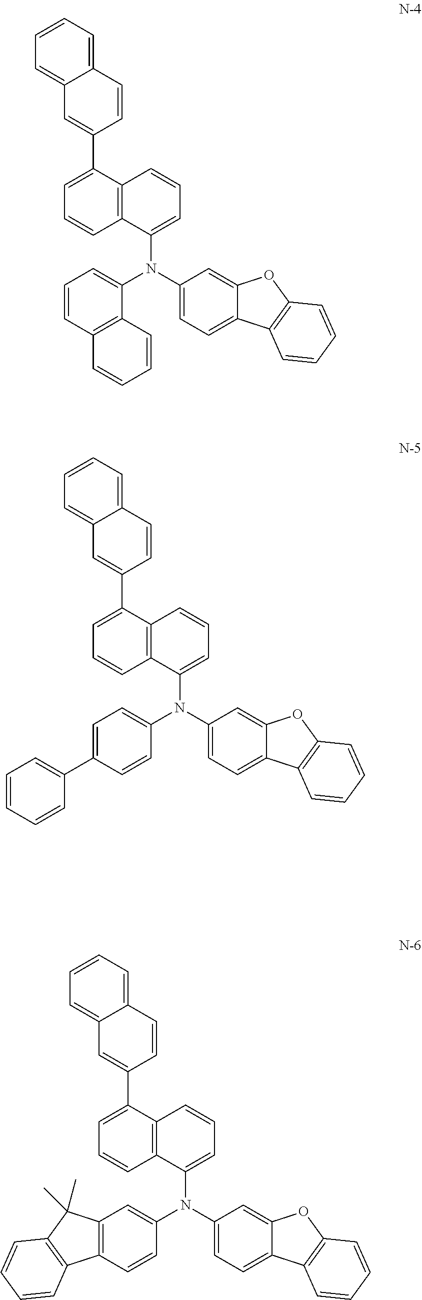 [SMILES: c1ccc2cc(-c3cccc4c(N(c5ccc6c(c5)oc5ccccc56)c5cccc6ccccc56)cccc34)ccc2c1 ; c1ccc(-c2ccc(N(c3ccc4c(c3)oc3ccccc34)c3cccc4c(-c5ccc6ccccc6c5)cccc34)cc2)cc1 ; CC1(C)c2ccccc2-c2ccc(N(c3ccc4c(c3)oc3ccccc34)c3cccc4c(-c5ccc6ccccc6c5)cccc34)cc21]

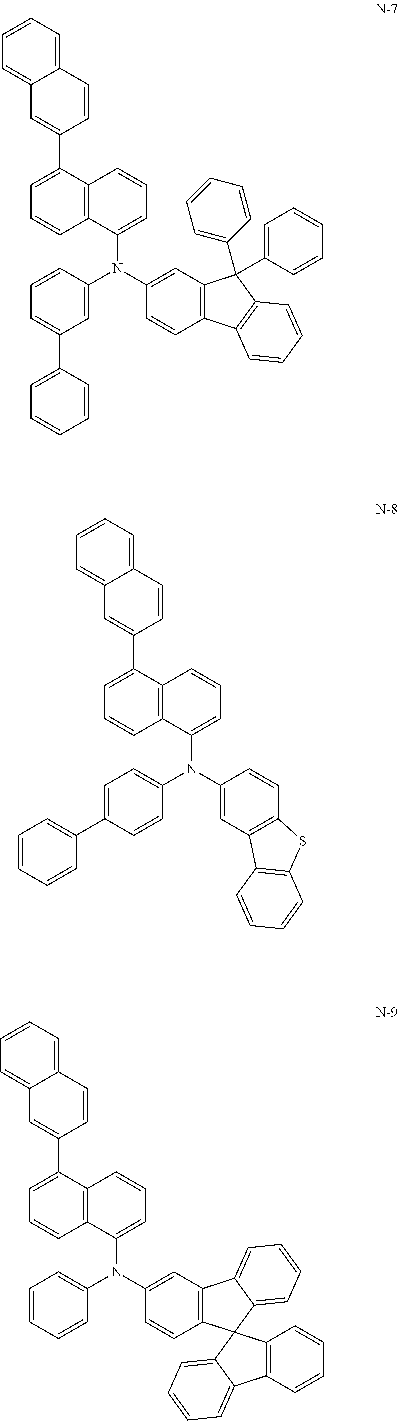 [SMILES: c1ccc(-c2cccc(N(c3ccc4c(c3)C(c3ccccc3)(c3ccccc3)c3ccccc3-4)c3cccc4c(-c5ccc6ccccc6c5)cccc34)c2)cc1 ; c1ccc(-c2ccc(N(c3ccc4sc5ccccc5c4c3)c3cccc4c(-c5ccc6ccccc6c5)cccc34)cc2)cc1 ; c1ccc(N(c2ccc3c(c2)-c2ccccc2C32c3ccccc3-c3ccccc32)c2cccc3c(-c4ccc5ccccc5c4)cccc23)cc1]

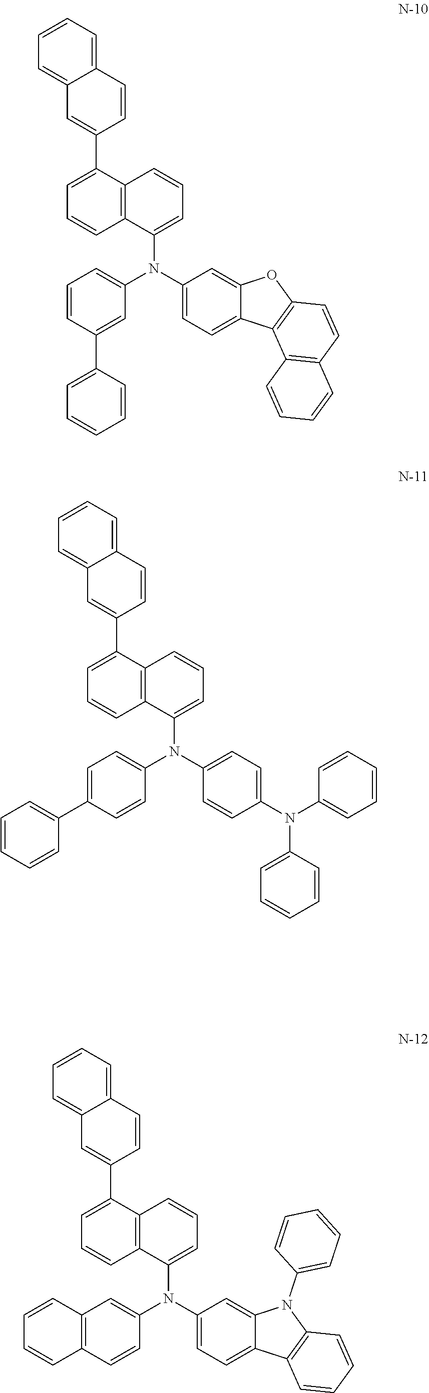 [SMILES: c1ccc(-c2cccc(N(c3ccc4c(c3)oc3ccc5ccccc5c34)c3cccc4c(-c5ccc6ccccc6c5)cccc34)c2)cc1 ; c1ccc(-c2ccc(N(c3ccc(N(c4ccccc4)c4ccccc4)cc3)c3cccc4c(-c5ccc6ccccc6c5)cccc34)cc2)cc1 ; c1ccc(-n2c3ccccc3c3ccc(N(c4ccc5ccccc5c4)c4cccc5c(-c6ccc7ccccc7c6)cccc45)cc32)cc1]

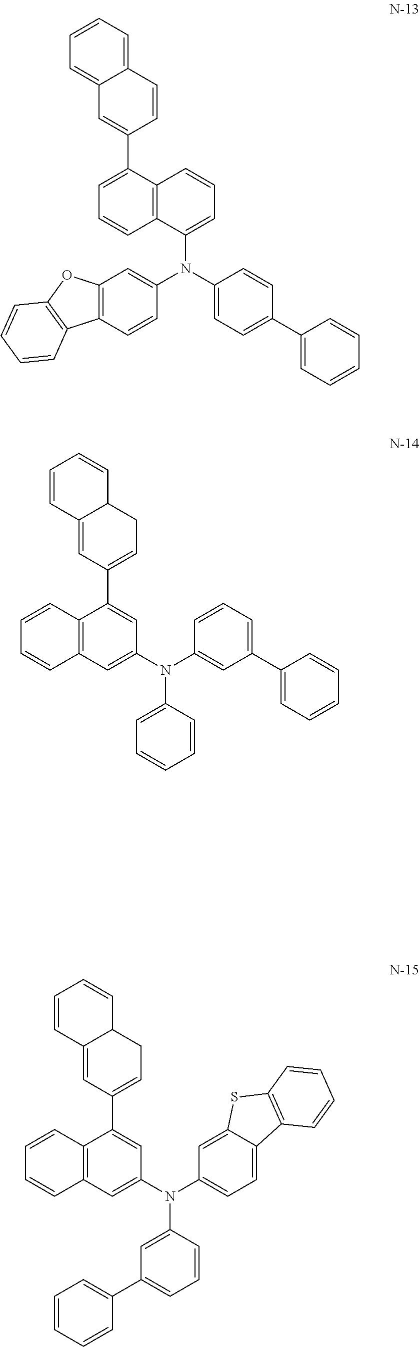 [SMILES: c1ccc(-c2ccc(N(c3ccc4c(c3)oc3ccccc34)c3cccc4c(-c5ccc6ccccc6c5)cccc34)cc2)cc1 ; C1=CC2=CC(c3cc(N(c4ccccc4)c4cccc(-c5ccccc5)c4)cc4ccccc34)=CCC2C=C1 ; C1=CC2=CC(c3cc(N(c4cccc(-c5ccccc5)c4)c4ccc5c(c4)sc4ccccc45)cc4ccccc34)=CCC2C=C1]

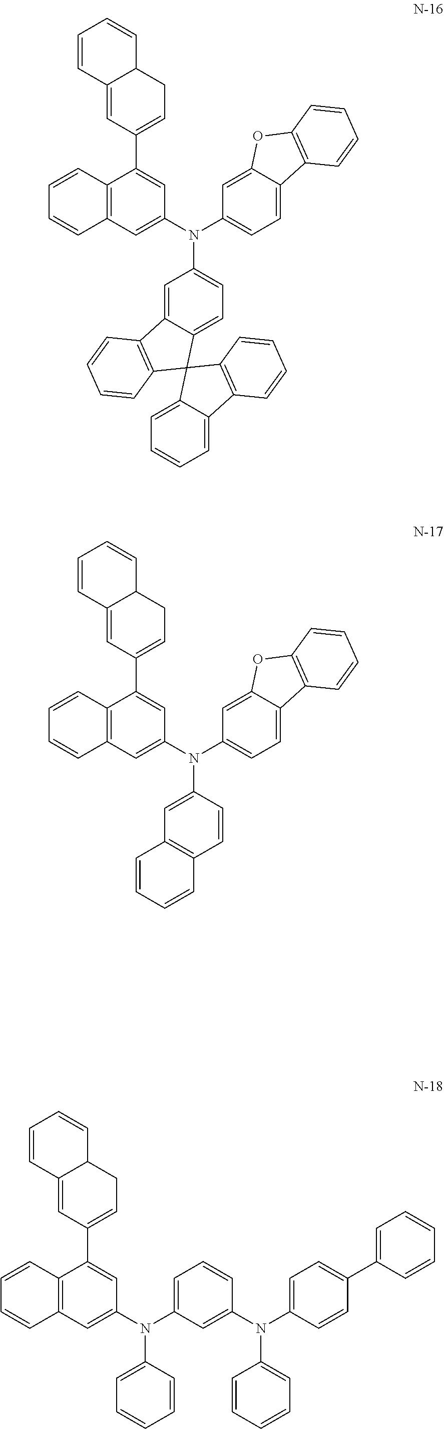 [SMILES: C1=CC2=CC(c3cc(N(c4ccc5c(c4)-c4ccccc4C54c5ccccc5-c5ccccc54)c4ccc5c(c4)oc4ccccc45)cc4ccccc34)=CCC2C=C1 ; C1=CC2=CC(c3cc(N(c4ccc5ccccc5c4)c4ccc5c(c4)oc4ccccc45)cc4ccccc34)=CCC2C=C1 ; C1=CC2=CC(c3cc(N(c4ccccc4)c4cccc(N(c5ccccc5)c5ccc(-c6ccccc6)cc5)c4)cc4ccccc34)=CCC2C=C1]

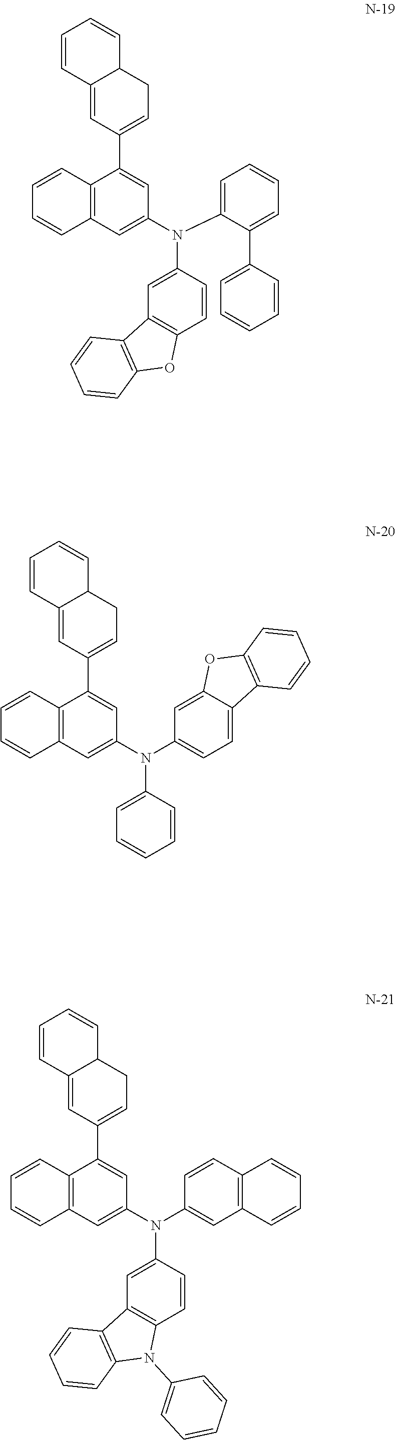 [SMILES: C1=CC2=CC(c3cc(N(c4ccc5oc6ccccc6c5c4)c4ccccc4-c4ccccc4)cc4ccccc34)=CCC2C=C1 ; C1=CC2=CC(c3cc(N(c4ccccc4)c4ccc5c(c4)oc4ccccc45)cc4ccccc34)=CCC2C=C1 ; C1=CC2=CC(c3cc(N(c4ccc5ccccc5c4)c4ccc5c(c4)c4ccccc4n5-c4ccccc4)cc4ccccc34)=CCC2C=C1]

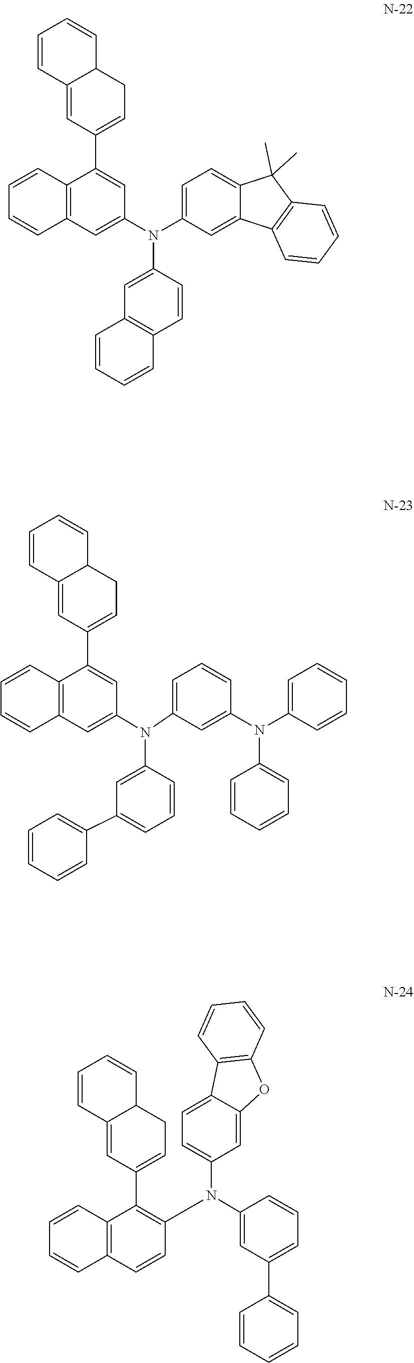 [SMILES: CC1(C)c2ccccc2-c2cc(N(c3ccc4ccccc4c3)c3cc(C4=CCC5C=CC=CC5=C4)c4ccccc4c3)ccc21 ; C1=CC2=CC(c3cc(N(c4cccc(-c5ccccc5)c4)c4cccc(N(c5ccccc5)c5ccccc5)c4)cc4ccccc34)=CCC2C=C1 ; C1=CC2=CC(c3c(N(c4cccc(-c5ccccc5)c4)c4ccc5c(c4)oc4ccccc45)ccc4ccccc34)=CCC2C=C1]

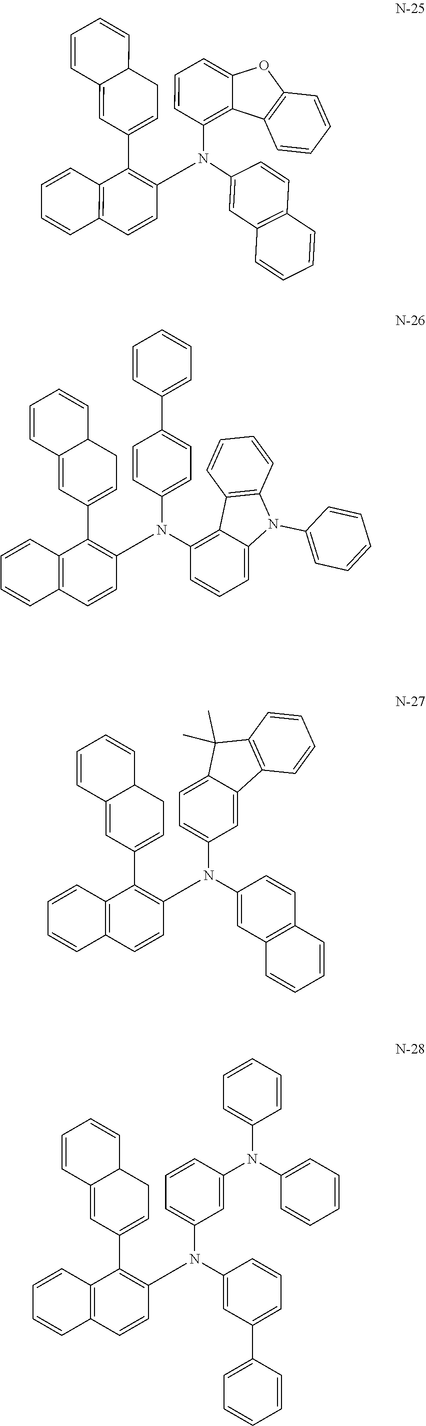 [SMILES: C1=CC2=CC(c3c(N(c4ccc5ccccc5c4)c4cccc5oc6ccccc6c45)ccc4ccccc34)=CCC2C=C1 ; C1=CC2=CC(c3c(N(c4ccc(-c5ccccc5)cc4)c4cccc5c4c4ccccc4n5-c4ccccc4)ccc4ccccc34)=CCC2C=C1 ; CC1(C)c2ccccc2-c2cc(N(c3ccc4ccccc4c3)c3ccc4ccccc4c3C3=CCC4C=CC=CC4=C3)ccc21 ; C1=CC2=CC(c3c(N(c4cccc(-c5ccccc5)c4)c4cccc(N(c5ccccc5)c5ccccc5)c4)ccc4ccccc34)=CCC2C=C1]

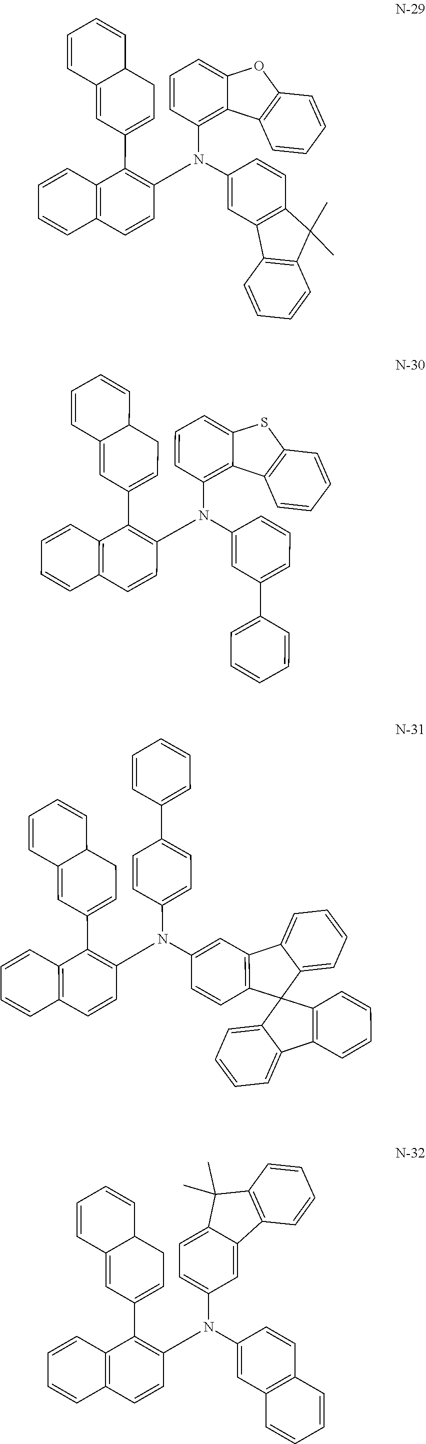 [SMILES: CC1(C)c2ccccc2-c2cc(N(c3ccc4ccccc4c3C3=CCC4C=CC=CC4=C3)c3cccc4oc5ccccc5c34)ccc21 ; C1=CC2=CC(c3c(N(c4cccc(-c5ccccc5)c4)c4cccc5sc6ccccc6c45)ccc4ccccc34)=CCC2C=C1 ; C1=CC2=CC(c3c(N(c4ccc(-c5ccccc5)cc4)c4ccc5c(c4)-c4ccccc4C54c5ccccc5-c5ccccc54)ccc4ccccc34)=CCC2C=C1 ; CC1(C)c2ccccc2-c2cc(N(c3ccc4ccccc4c3)c3ccc4ccccc4c3C3=CCC4C=CC=CC4=C3)ccc21]

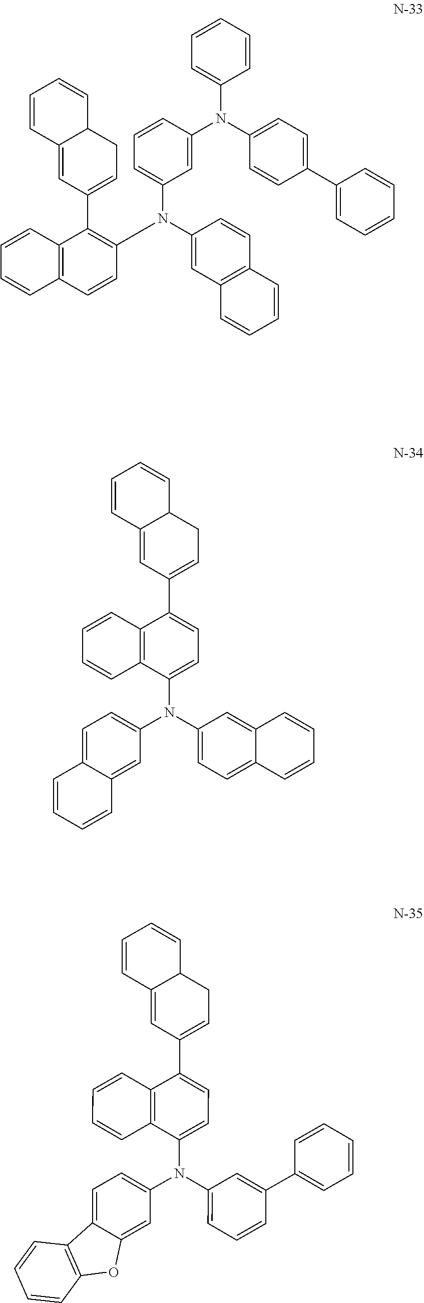 [SMILES: C1=CC2=CC(c3c(N(c4cccc(N(c5ccccc5)c5ccc(-c6ccccc6)cc5)c4)c4ccc5ccccc5c4)ccc4ccccc34)=CCC2C=C1 ; C1=CC2=CC(c3ccc(N(c4ccc5ccccc5c4)c4ccc5ccccc5c4)c4ccccc34)=CCC2C=C1 ; C1=CC2=CC(c3ccc(N(c4cccc(-c5ccccc5)c4)c4ccc5c(c4)oc4ccccc45)c4ccccc34)=CCC2C=C1]

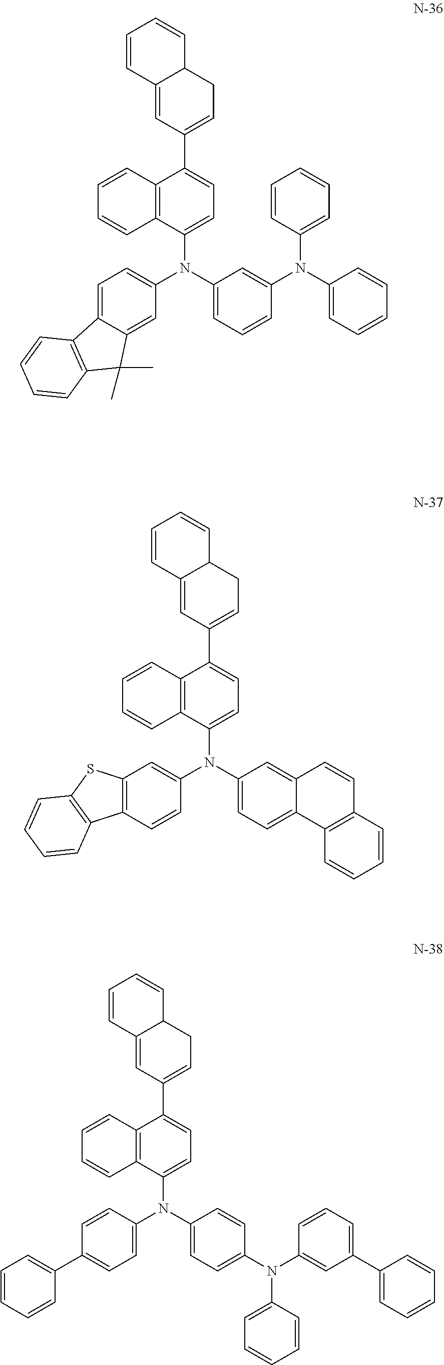 [SMILES: CC1(C)c2ccccc2-c2ccc(N(c3cccc(N(c4ccccc4)c4ccccc4)c3)c3ccc(C4=CCC5C=CC=CC5=C4)c4ccccc34)cc21 ; C1=CC2=CC(c3ccc(N(c4ccc5c(ccc6ccccc65)c4)c4ccc5c(c4)sc4ccccc45)c4ccccc34)=CCC2C=C1 ; C1=CC2=CC(c3ccc(N(c4ccc(-c5ccccc5)cc4)c4ccc(N(c5ccccc5)c5cccc(-c6ccccc6)c5)cc4)c4ccccc34)=CCC2C=C1]

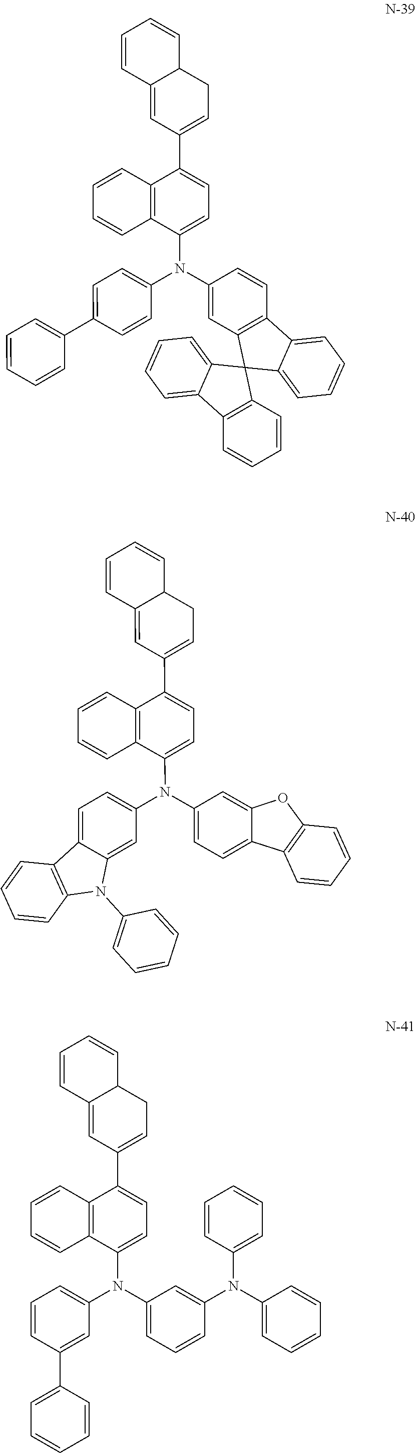 [SMILES: C1=CC2=CC(c3ccc(N(c4ccc(-c5ccccc5)cc4)c4ccc5c(c4)C4(c6ccccc6-c6ccccc64)c4ccccc4-5)c4ccccc34)=CCC2C=C1 ; C1=CC2=CC(c3ccc(N(c4ccc5c(c4)oc4ccccc45)c4ccc5c6ccccc6n(-c6ccccc6)c5c4)c4ccccc34)=CCC2C=C1 ; C1=CC2=CC(c3ccc(N(c4cccc(-c5ccccc5)c4)c4cccc(N(c5ccccc5)c5ccccc5)c4)c4ccccc34)=CCC2C=C1]

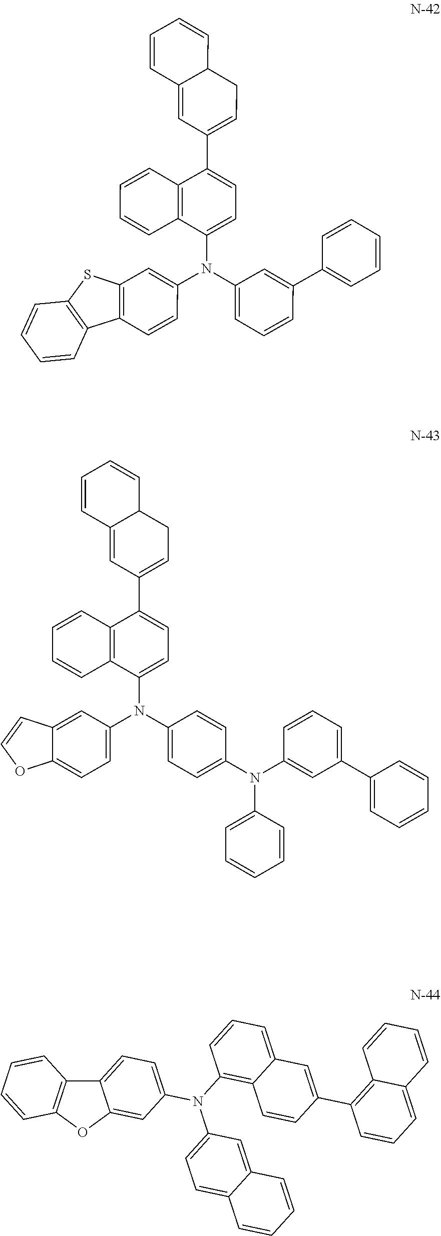 [SMILES: C1=CC2=CC(c3ccc(N(c4cccc(-c5ccccc5)c4)c4ccc5c(c4)sc4ccccc45)c4ccccc34)=CCC2C=C1 ; C1=CC2=CC(c3ccc(N(c4ccc(N(c5ccccc5)c5cccc(-c6ccccc6)c5)cc4)c4ccc5occc5c4)c4ccccc34)=CCC2C=C1 ; c1ccc2cc(N(c3ccc4c(c3)oc3ccccc34)c3cccc4cc(-c5cccc6ccccc56)ccc34)ccc2c1]

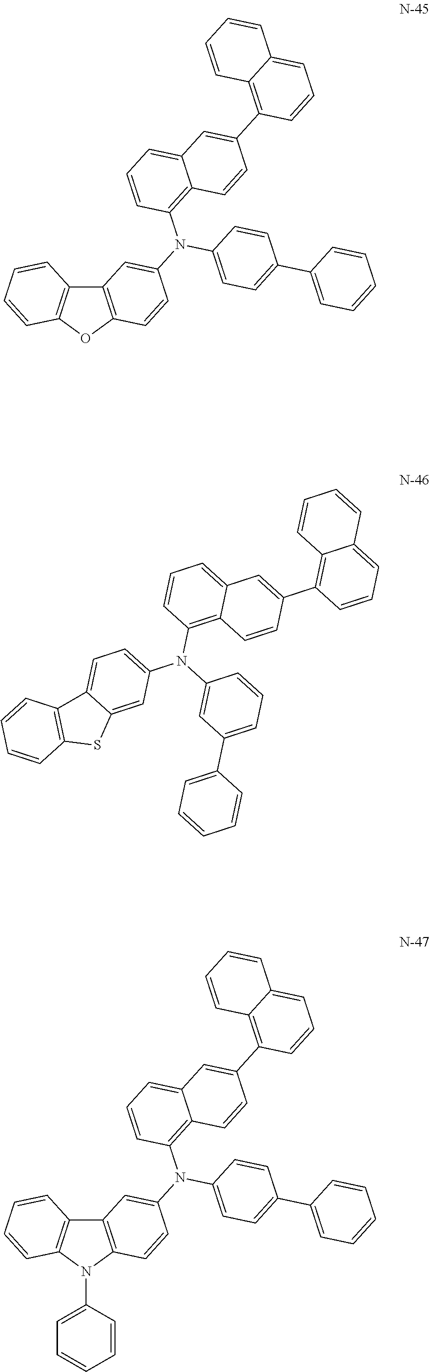 [SMILES: c1ccc(-c2ccc(N(c3ccc4oc5ccccc5c4c3)c3cccc4cc(-c5cccc6ccccc56)ccc34)cc2)cc1 ; c1ccc(-c2cccc(N(c3ccc4c(c3)sc3ccccc34)c3cccc4cc(-c5cccc6ccccc56)ccc34)c2)cc1 ; c1ccc(-c2ccc(N(c3ccc4c(c3)c3ccccc3n4-c3ccccc3)c3cccc4cc(-c5cccc6ccccc56)ccc34)cc2)cc1]

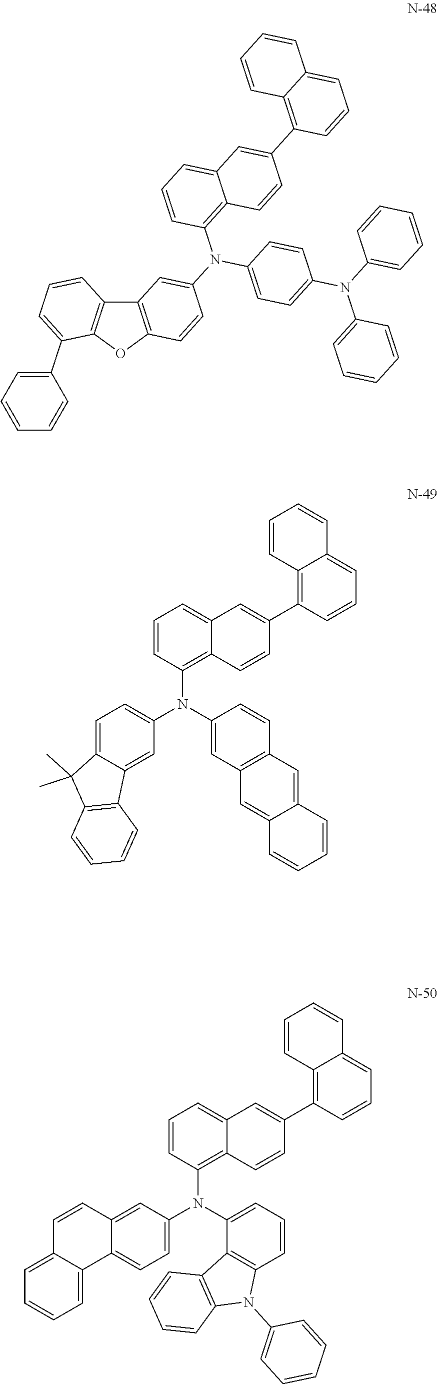 [SMILES: c1ccc(-c2cccc3c2oc2ccc(N(c4ccc(N(c5ccccc5)c5ccccc5)cc4)c4cccc5cc(-c6cccc7ccccc67)ccc45)cc23)cc1 ; CC1(C)c2ccccc2-c2cc(N(c3ccc4cc5ccccc5cc4c3)c3cccc4cc(-c5cccc6ccccc56)ccc34)ccc21 ; c1ccc(-n2c3ccccc3c3c(N(c4ccc5c(ccc6ccccc65)c4)c4cccc5cc(-c6cccc7ccccc67)ccc45)cccc32)cc1]

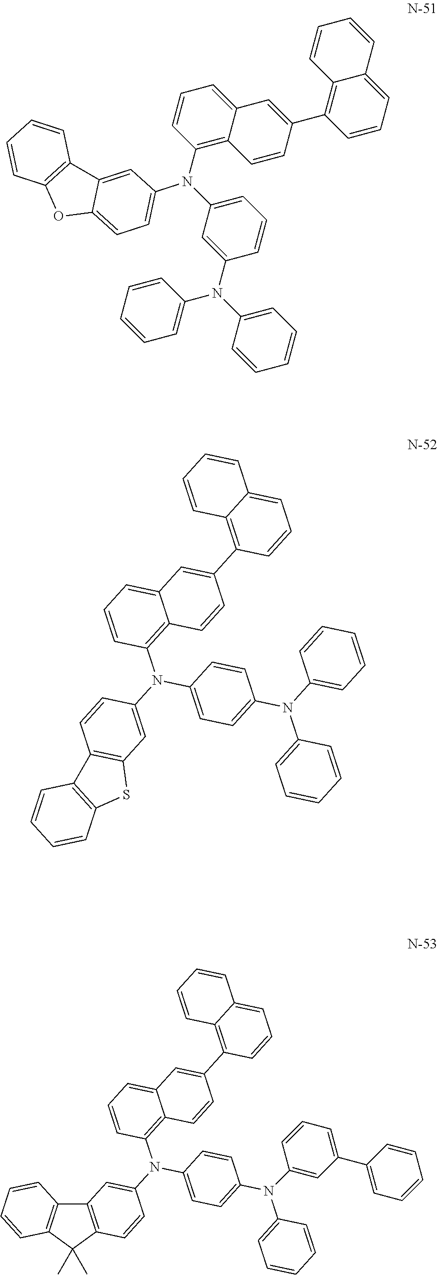 [SMILES: c1ccc(N(c2ccccc2)c2cccc(N(c3ccc4oc5ccccc5c4c3)c3cccc4cc(-c5cccc6ccccc56)ccc34)c2)cc1 ; c1ccc(N(c2ccccc2)c2ccc(N(c3ccc4c(c3)sc3ccccc34)c3cccc4cc(-c5cccc6ccccc56)ccc34)cc2)cc1 ; CC1(C)c2ccccc2-c2cc(N(c3ccc(N(c4ccccc4)c4cccc(-c5ccccc5)c4)cc3)c3cccc4cc(-c5cccc6ccccc56)ccc34)ccc21]

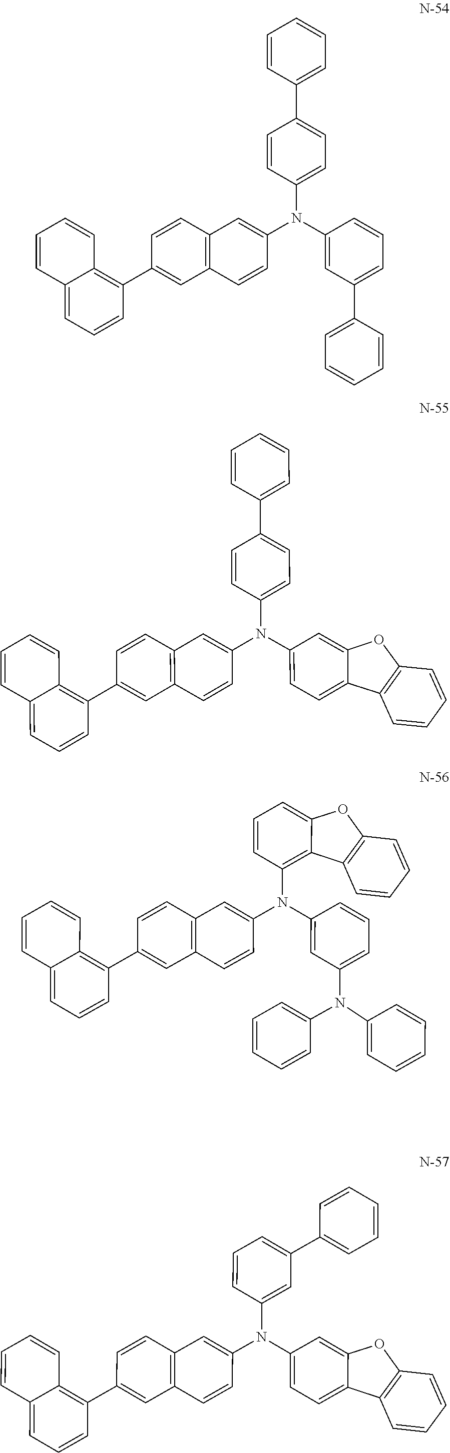 [SMILES: c1ccc(-c2ccc(N(c3cccc(-c4ccccc4)c3)c3ccc4cc(-c5cccc6ccccc56)ccc4c3)cc2)cc1 ; c1ccc(-c2ccc(N(c3ccc4cc(-c5cccc6ccccc56)ccc4c3)c3ccc4c(c3)oc3ccccc34)cc2)cc1 ; c1ccc(N(c2ccccc2)c2cccc(N(c3ccc4cc(-c5cccc6ccccc56)ccc4c3)c3cccc4oc5ccccc5c34)c2)cc1 ; c1ccc(-c2cccc(N(c3ccc4cc(-c5cccc6ccccc56)ccc4c3)c3ccc4c(c3)oc3ccccc34)c2)cc1]

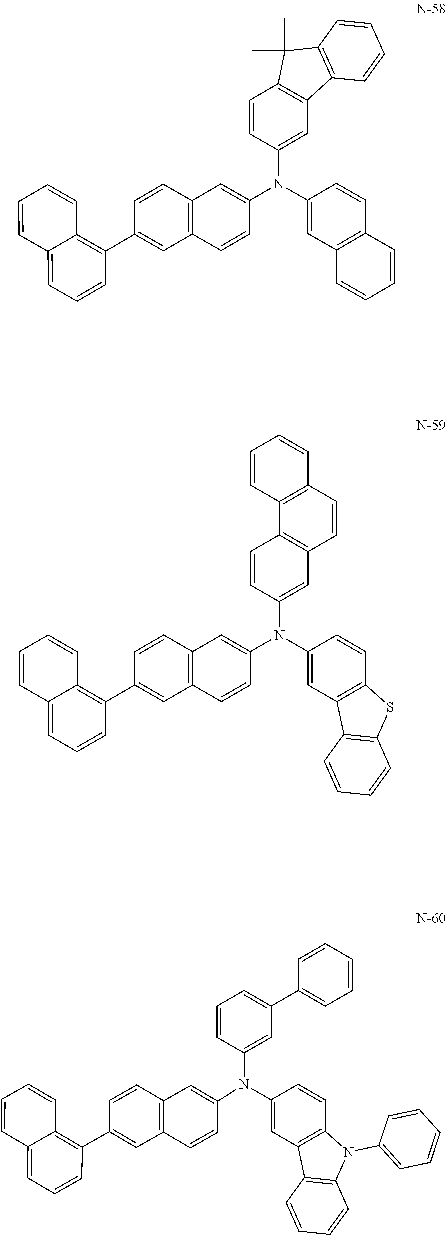 [SMILES: CC1(C)c2ccccc2-c2cc(N(c3ccc4ccccc4c3)c3ccc4cc(-c5cccc6ccccc56)ccc4c3)ccc21 ; c1ccc2c(-c3ccc4cc(N(c5ccc6c(ccc7ccccc76)c5)c5ccc6sc7ccccc7c6c5)ccc4c3)cccc2c1 ; c1ccc(-c2cccc(N(c3ccc4cc(-c5cccc6ccccc56)ccc4c3)c3ccc4c(c3)c3ccccc3n4-c3ccccc3)c2)cc1]

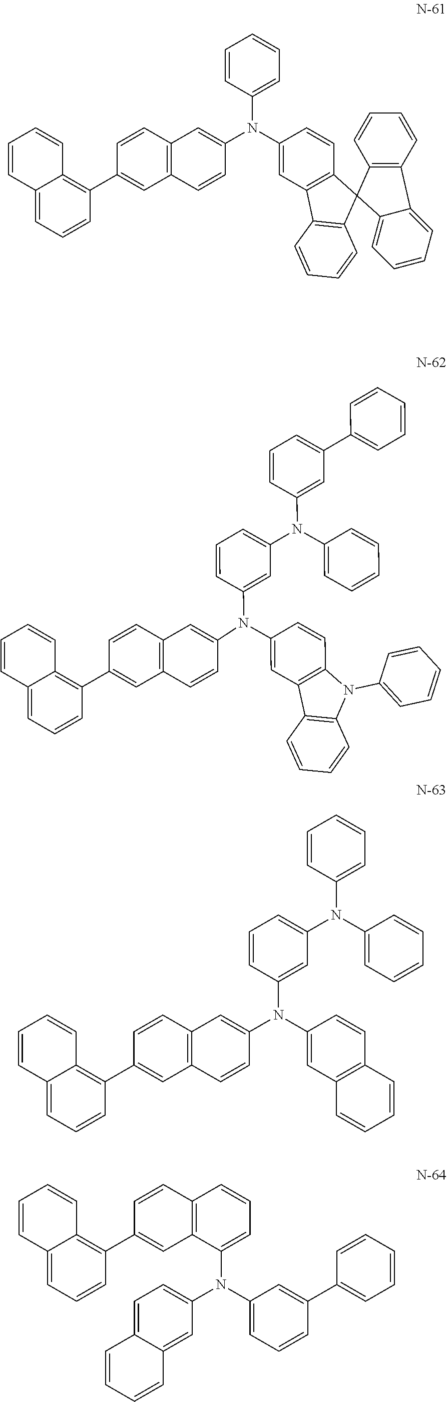

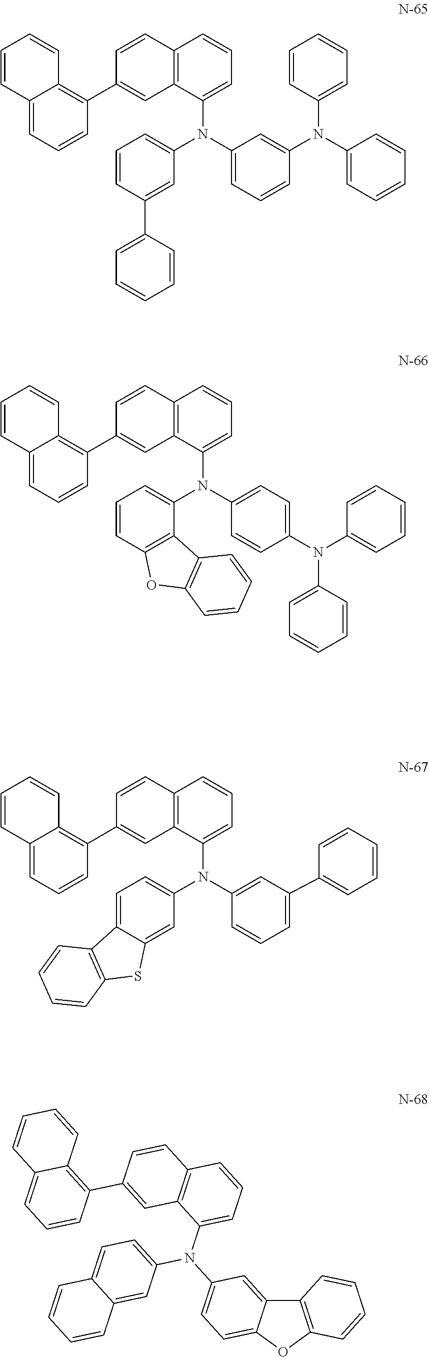 [SMILES: c1ccc(-c2cccc(N(c3cccc(N(c4ccccc4)c4ccccc4)c3)c3cccc4ccc(-c5cccc6ccccc56)cc34)c2)cc1 ; c1ccc(N(c2ccccc2)c2ccc(N(c3cccc4ccc(-c5cccc6ccccc56)cc34)c3cccc4oc5ccccc5c34)cc2)cc1 ; c1ccc(-c2cccc(N(c3ccc4c(c3)sc3ccccc34)c3cccc4ccc(-c5cccc6ccccc56)cc34)c2)cc1 ; c1ccc2cc(N(c3ccc4oc5ccccc5c4c3)c3cccc4ccc(-c5cccc6ccccc56)cc34)ccc2c1]

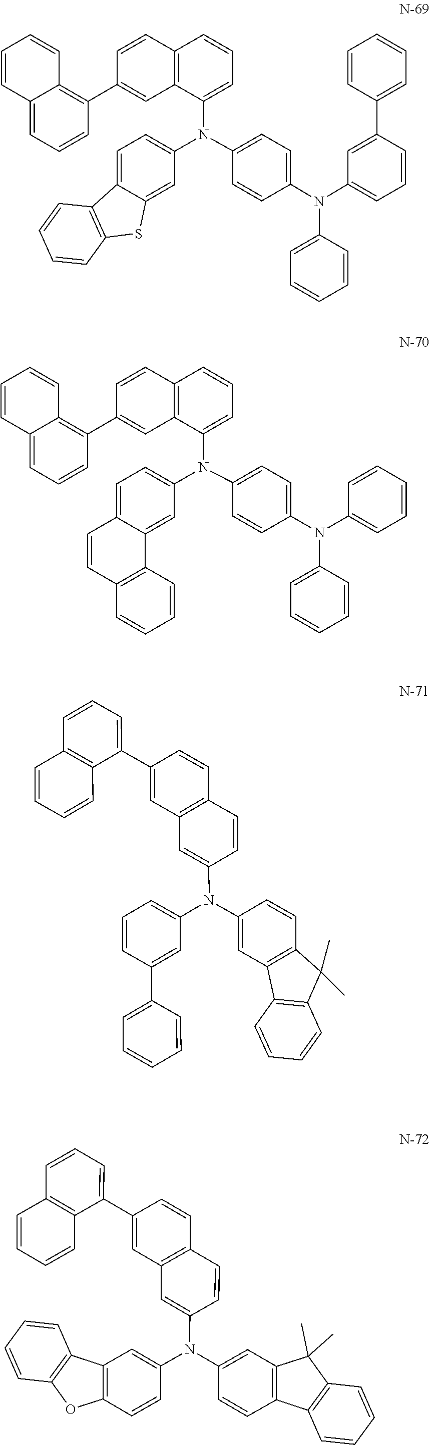 [SMILES: c1ccc(-c2cccc(N(c3ccccc3)c3ccc(N(c4ccc5c(c4)sc4ccccc45)c4cccc5ccc(-c6cccc7ccccc67)cc45)cc3)c2)cc1 ; c1ccc(N(c2ccccc2)c2ccc(N(c3ccc4ccc5ccccc5c4c3)c3cccc4ccc(-c5cccc6ccccc56)cc34)cc2)cc1 ; CC1(C)c2ccccc2-c2cc(N(c3cccc(-c4ccccc4)c3)c3ccc4ccc(-c5cccc6ccccc56)cc4c3)ccc21 ; CC1(C)c2ccccc2-c2ccc(N(c3ccc4ccc(-c5cccc6ccccc56)cc4c3)c3ccc4oc5ccccc5c4c3)cc21]

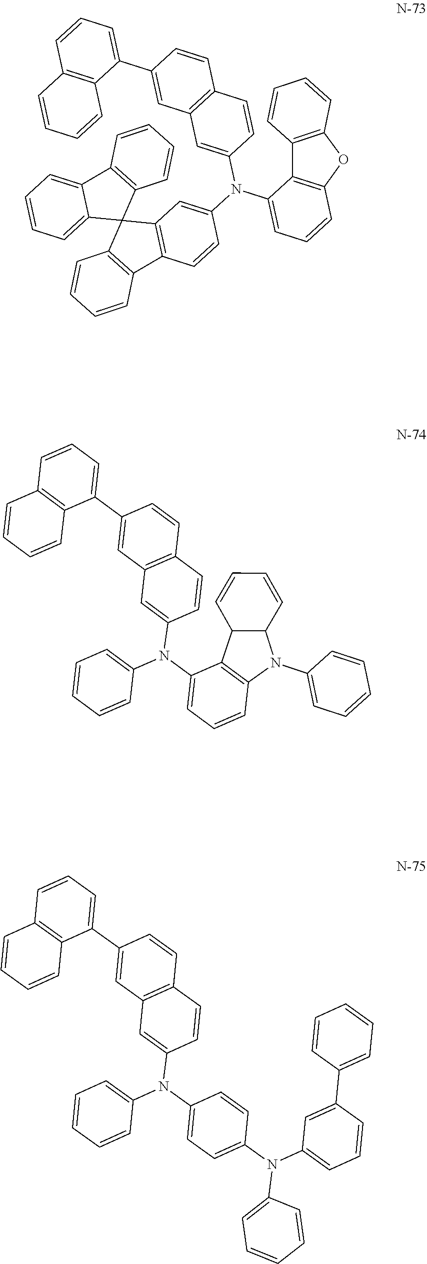 [SMILES: c1ccc2c(c1)-c1ccccc1C21c2ccccc2-c2ccc(N(c3ccc4ccc(-c5cccc6ccccc56)cc4c3)c3cccc4oc5ccccc5c34)cc21 ; C1=CC2c3c(N(c4ccccc4)c4ccc5ccc(-c6cccc7ccccc67)cc5c4)cccc3N(c3ccccc3)C2C=C1 ; c1ccc(-c2cccc(N(c3ccccc3)c3ccc(N(c4ccccc4)c4ccc5ccc(-c6cccc7ccccc67)cc5c4)cc3)c2)cc1]

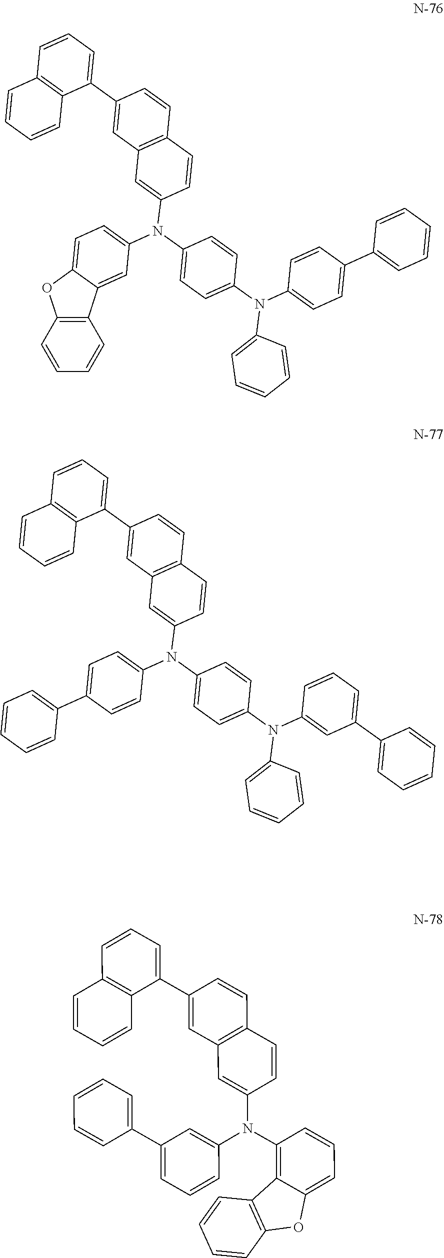 [SMILES: c1ccc(-c2ccc(N(c3ccccc3)c3ccc(N(c4ccc5ccc(-c6cccc7ccccc67)cc5c4)c4ccc5oc6ccccc6c5c4)cc3)cc2)cc1 ; c1ccc(-c2ccc(N(c3ccc(N(c4ccccc4)c4cccc(-c5ccccc5)c4)cc3)c3ccc4ccc(-c5cccc6ccccc56)cc4c3)cc2)cc1 ; c1ccc(-c2cccc(N(c3ccc4ccc(-c5cccc6ccccc56)cc4c3)c3cccc4oc5ccccc5c34)c2)cc1]

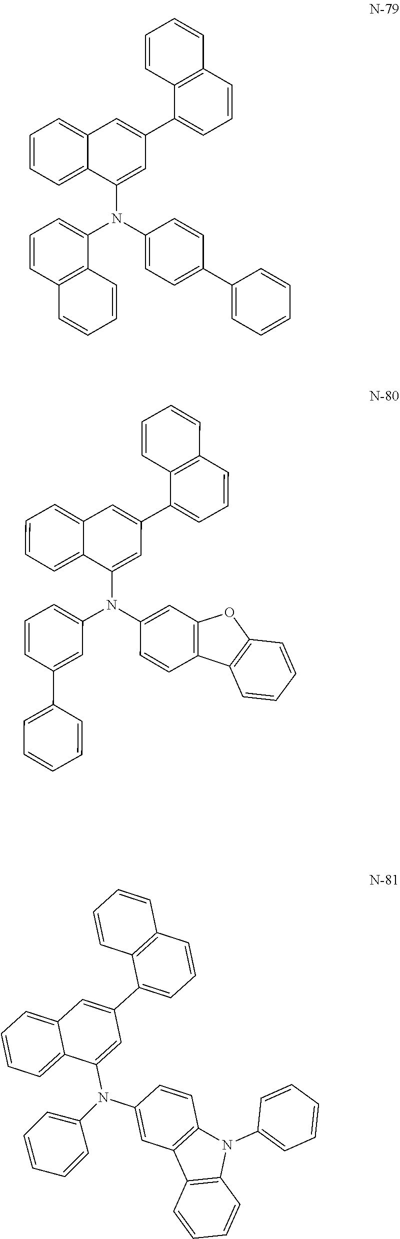 [SMILES: c1ccc(-c2ccc(N(c3cccc4ccccc34)c3cc(-c4cccc5ccccc45)cc4ccccc34)cc2)cc1 ; c1ccc(-c2cccc(N(c3ccc4c(c3)oc3ccccc34)c3cc(-c4cccc5ccccc45)cc4ccccc34)c2)cc1 ; c1ccc(N(c2ccc3c(c2)c2ccccc2n3-c2ccccc2)c2cc(-c3cccc4ccccc34)cc3ccccc23)cc1]

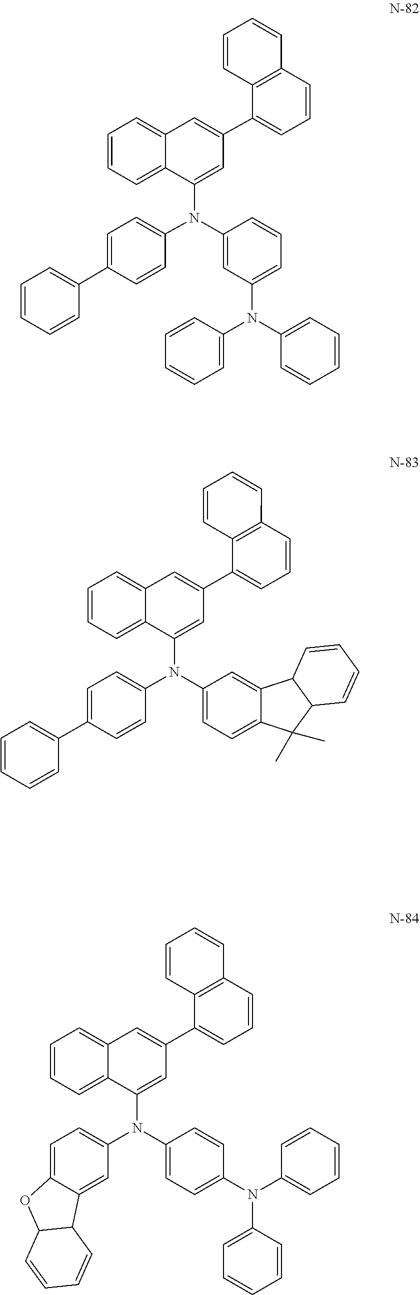 [SMILES: c1ccc(-c2ccc(N(c3cccc(N(c4ccccc4)c4ccccc4)c3)c3cc(-c4cccc5ccccc45)cc4ccccc34)cc2)cc1 ; CC1(C)c2ccc(N(c3ccc(-c4ccccc4)cc3)c3cc(-c4cccc5ccccc45)cc4ccccc34)cc2C2C=CC=CC21 ; C1=CC2Oc3ccc(N(c4ccc(N(c5ccccc5)c5ccccc5)cc4)c4cc(-c5cccc6ccccc56)cc5ccccc45)cc3C2C=C1]

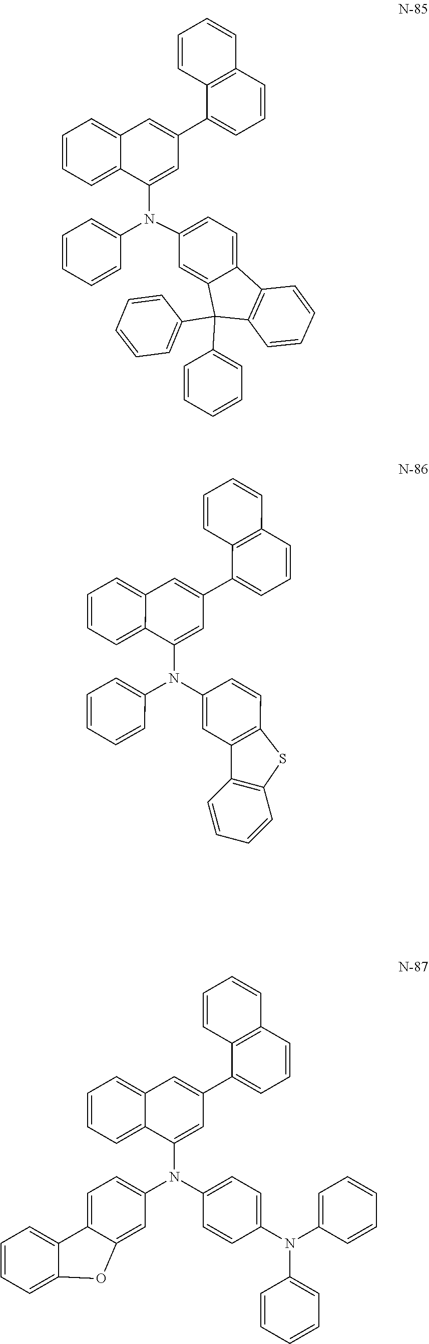 [SMILES: c1ccc(N(c2ccc3c(c2)C(c2ccccc2)(c2ccccc2)c2ccccc2-3)c2cc(-c3cccc4ccccc34)cc3ccccc23)cc1 ; c1ccc(N(c2ccc3sc4ccccc4c3c2)c2cc(-c3cccc4ccccc34)cc3ccccc23)cc1 ; c1ccc(N(c2ccccc2)c2ccc(N(c3ccc4c(c3)oc3ccccc34)c3cc(-c4cccc5ccccc45)cc4ccccc34)cc2)cc1]

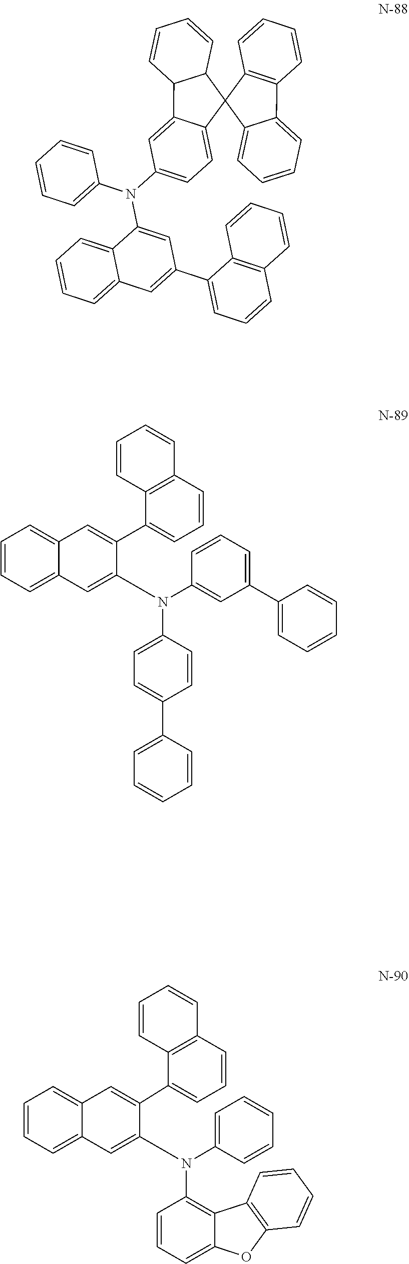 [SMILES: C1=CC2c3cc(N(c4ccccc4)c4cc(-c5cccc6ccccc56)cc5ccccc45)ccc3C3(c4ccccc4-c4ccccc43)C2C=C1 ; c1ccc(-c2ccc(N(c3cccc(-c4ccccc4)c3)c3cc4ccccc4cc3-c3cccc4ccccc34)cc2)cc1 ; c1ccc(N(c2cc3ccccc3cc2-c2cccc3ccccc23)c2cccc3oc4ccccc4c23)cc1]

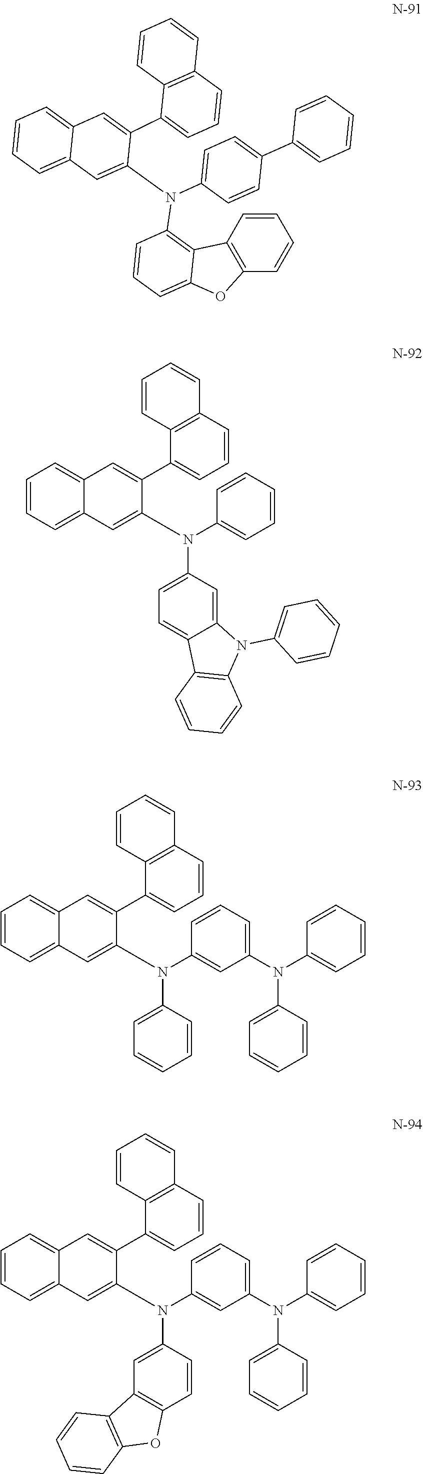 [SMILES: c1ccc(-c2ccc(N(c3cc4ccccc4cc3-c3cccc4ccccc34)c3cccc4oc5ccccc5c34)cc2)cc1 ; c1ccc(N(c2ccc3c4ccccc4n(-c4ccccc4)c3c2)c2cc3ccccc3cc2-c2cccc3ccccc23)cc1 ; c1ccc(N(c2ccccc2)c2cccc(N(c3ccccc3)c3cc4ccccc4cc3-c3cccc4ccccc34)c2)cc1 ; c1ccc(N(c2ccccc2)c2cccc(N(c3ccc4oc5ccccc5c4c3)c3cc4ccccc4cc3-c3cccc4ccccc34)c2)cc1]

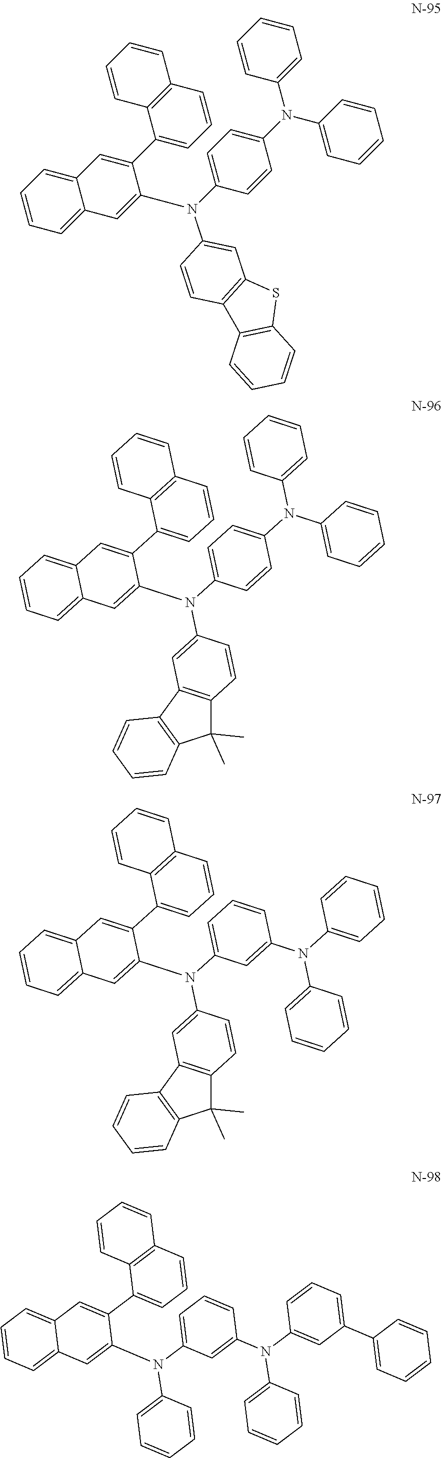

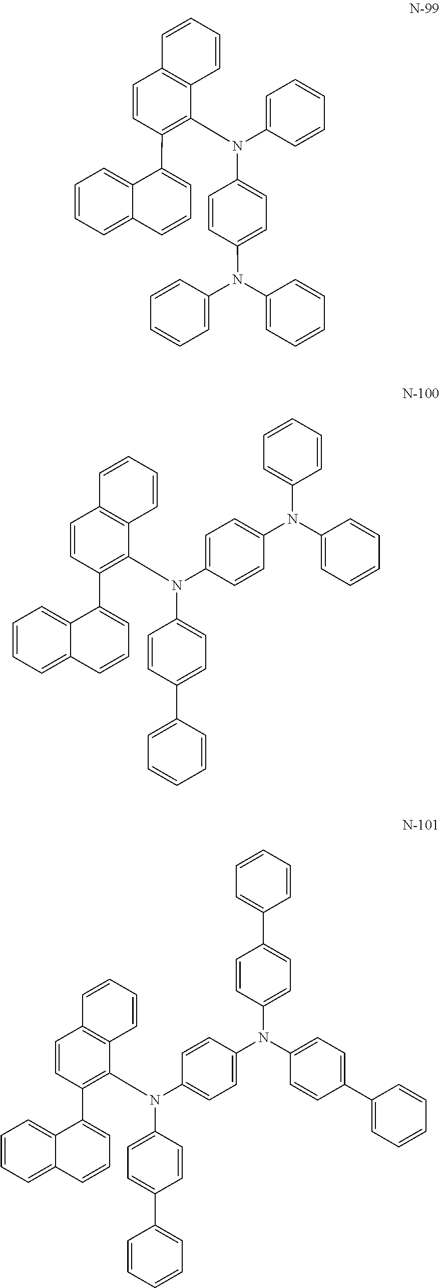 [SMILES: c1ccc(N(c2ccccc2)c2ccc(N(c3ccccc3)c3c(-c4cccc5ccccc45)ccc4ccccc34)cc2)cc1 ; c1ccc(-c2ccc(N(c3ccc(N(c4ccccc4)c4ccccc4)cc3)c3c(-c4cccc5ccccc45)ccc4ccccc34)cc2)cc1 ; c1ccc(-c2ccc(N(c3ccc(-c4ccccc4)cc3)c3ccc(N(c4ccc(-c5ccccc5)cc4)c4c(-c5cccc6ccccc56)ccc5ccccc45)cc3)cc2)cc1]

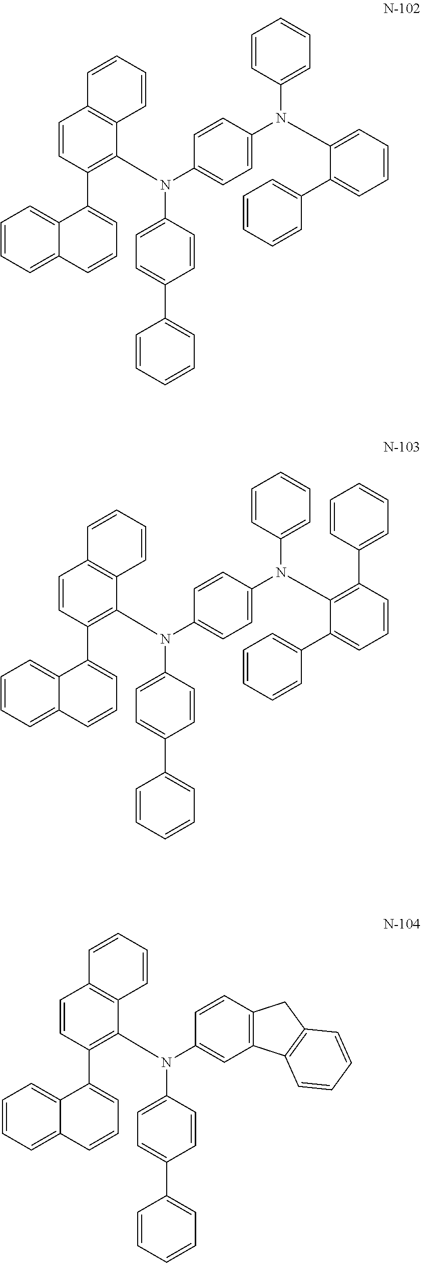 [SMILES: c1ccc(-c2ccc(N(c3ccc(N(c4ccccc4)c4ccccc4-c4ccccc4)cc3)c3c(-c4cccc5ccccc45)ccc4ccccc34)cc2)cc1 ; c1ccc(-c2ccc(N(c3ccc(N(c4ccccc4)c4c(-c5ccccc5)cccc4-c4ccccc4)cc3)c3c(-c4cccc5ccccc45)ccc4ccccc34)cc2)cc1 ; c1ccc(-c2ccc(N(c3ccc4c(c3)-c3ccccc3C4)c3c(-c4cccc5ccccc45)ccc4ccccc34)cc2)cc1]

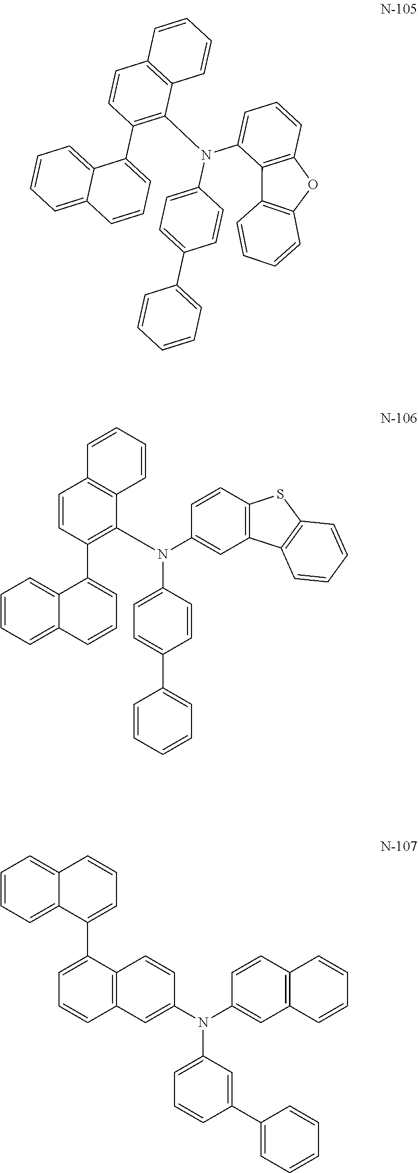 [SMILES: c1ccc(-c2ccc(N(c3c(-c4cccc5ccccc45)ccc4ccccc34)c3cccc4oc5ccccc5c34)cc2)cc1 ; c1ccc(-c2ccc(N(c3ccc4sc5ccccc5c4c3)c3c(-c4cccc5ccccc45)ccc4ccccc34)cc2)cc1 ; c1ccc(-c2cccc(N(c3ccc4ccccc4c3)c3ccc4c(-c5cccc6ccccc56)cccc4c3)c2)cc1]

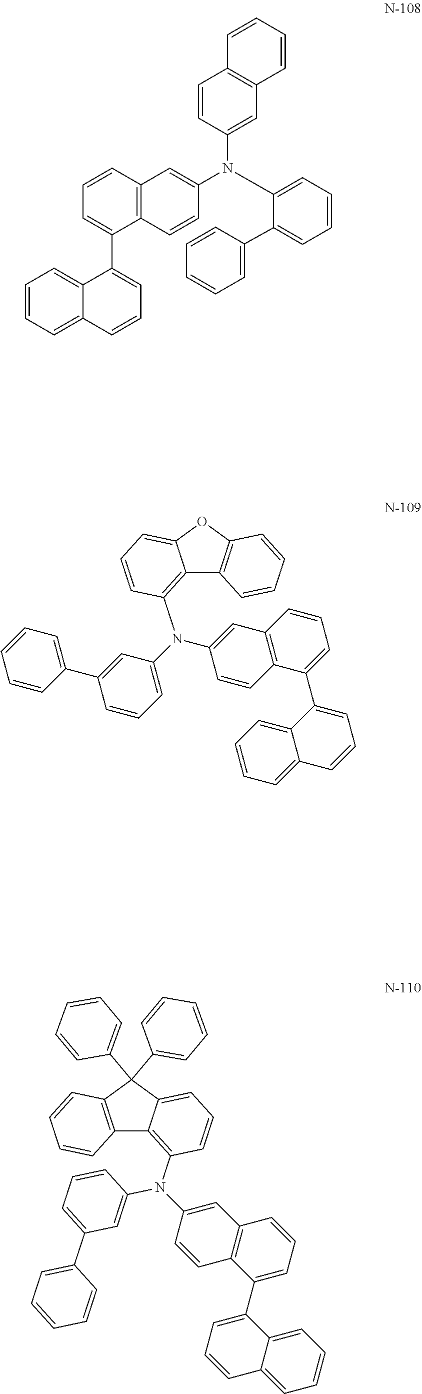 [SMILES: c1ccc(-c2ccccc2N(c2ccc3ccccc3c2)c2ccc3c(-c4cccc5ccccc45)cccc3c2)cc1 ; c1ccc(-c2cccc(N(c3ccc4c(-c5cccc6ccccc56)cccc4c3)c3cccc4oc5ccccc5c34)c2)cc1 ; c1ccc(-c2cccc(N(c3ccc4c(-c5cccc6ccccc56)cccc4c3)c3cccc4c3-c3ccccc3C4(c3ccccc3)c3ccccc3)c2)cc1]

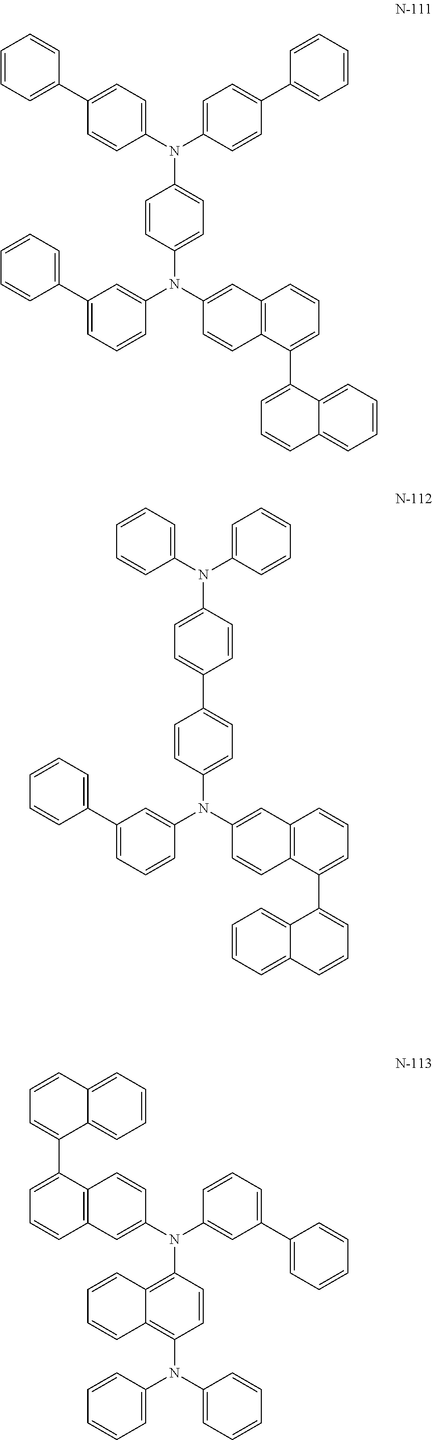 [SMILES: c1ccc(-c2ccc(N(c3ccc(-c4ccccc4)cc3)c3ccc(N(c4cccc(-c5ccccc5)c4)c4ccc5c(-c6cccc7ccccc67)cccc5c4)cc3)cc2)cc1 ; c1ccc(-c2cccc(N(c3ccc(-c4ccc(N(c5ccccc5)c5ccccc5)cc4)cc3)c3ccc4c(-c5cccc6ccccc56)cccc4c3)c2)cc1 ; c1ccc(-c2cccc(N(c3ccc4c(-c5cccc6ccccc56)cccc4c3)c3ccc(N(c4ccccc4)c4ccccc4)c4ccccc34)c2)cc1]

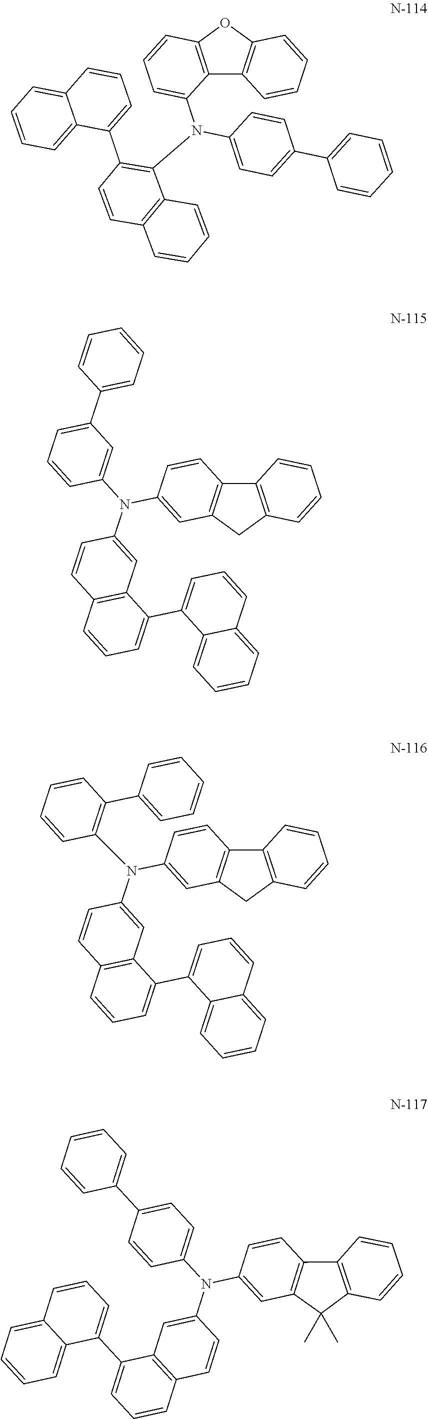 [SMILES: c1ccc(-c2ccc(N(c3c(-c4cccc5ccccc45)ccc4ccccc34)c3cccc4oc5ccccc5c34)cc2)cc1 ; c1ccc(-c2cccc(N(c3ccc4c(c3)Cc3ccccc3-4)c3ccc4cccc(-c5cccc6ccccc56)c4c3)c2)cc1 ; c1ccc(-c2ccccc2N(c2ccc3c(c2)Cc2ccccc2-3)c2ccc3cccc(-c4cccc5ccccc45)c3c2)cc1 ; CC1(C)c2ccccc2-c2ccc(N(c3ccc(-c4ccccc4)cc3)c3ccc4cccc(-c5cccc6ccccc56)c4c3)cc21]

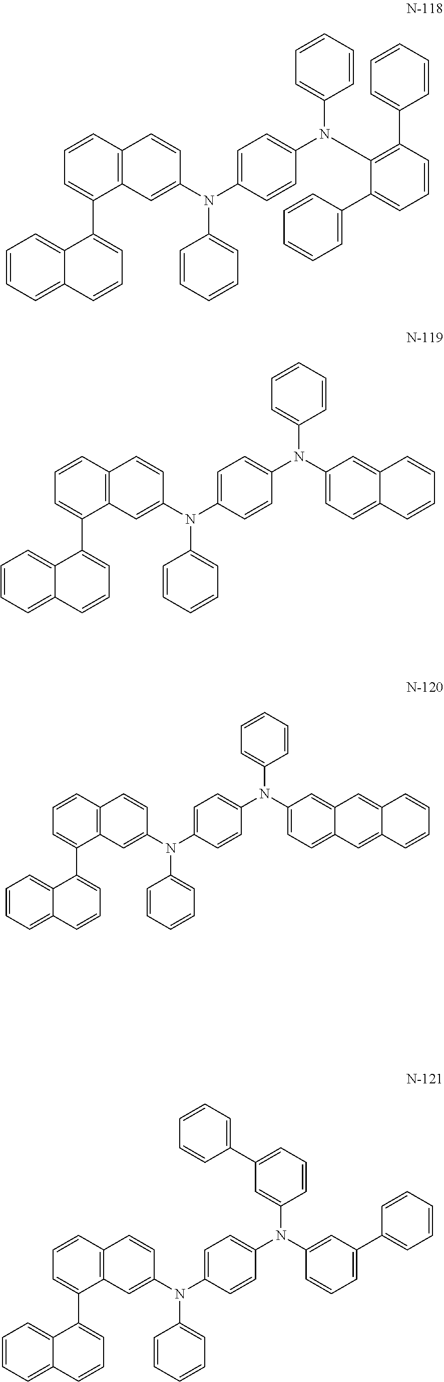 [SMILES: c1ccc(-c2cccc(-c3ccccc3)c2N(c2ccccc2)c2ccc(N(c3ccccc3)c3ccc4cccc(-c5cccc6ccccc56)c4c3)cc2)cc1 ; c1ccc(N(c2ccc(N(c3ccccc3)c3ccc4cccc(-c5cccc6ccccc56)c4c3)cc2)c2ccc3ccccc3c2)cc1 ; c1ccc(N(c2ccc(N(c3ccccc3)c3ccc4cccc(-c5cccc6ccccc56)c4c3)cc2)c2ccc3cc4ccccc4cc3c2)cc1 ; c1ccc(-c2cccc(N(c3ccc(N(c4ccccc4)c4ccc5cccc(-c6cccc7ccccc67)c5c4)cc3)c3cccc(-c4ccccc4)c3)c2)cc1]

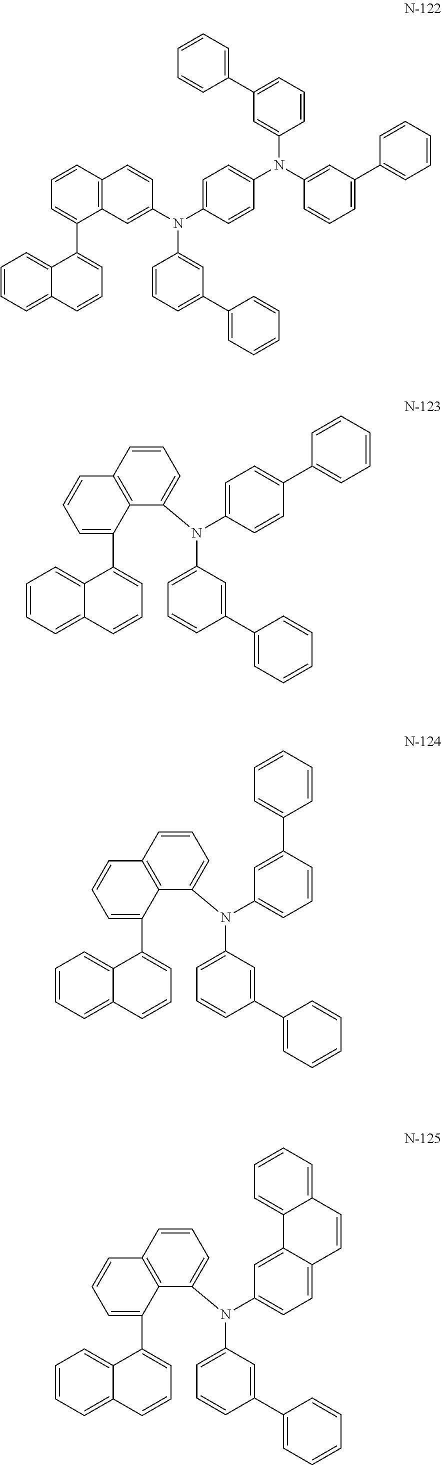 [SMILES: c1ccc(-c2cccc(N(c3ccc(N(c4cccc(-c5ccccc5)c4)c4ccc5cccc(-c6cccc7ccccc67)c5c4)cc3)c3cccc(-c4ccccc4)c3)c2)cc1 ; c1ccc(-c2ccc(N(c3cccc(-c4ccccc4)c3)c3cccc4cccc(-c5cccc6ccccc56)c34)cc2)cc1 ; c1ccc(-c2cccc(N(c3cccc(-c4ccccc4)c3)c3cccc4cccc(-c5cccc6ccccc56)c34)c2)cc1 ; c1ccc(-c2cccc(N(c3ccc4ccc5ccccc5c4c3)c3cccc4cccc(-c5cccc6ccccc56)c34)c2)cc1]

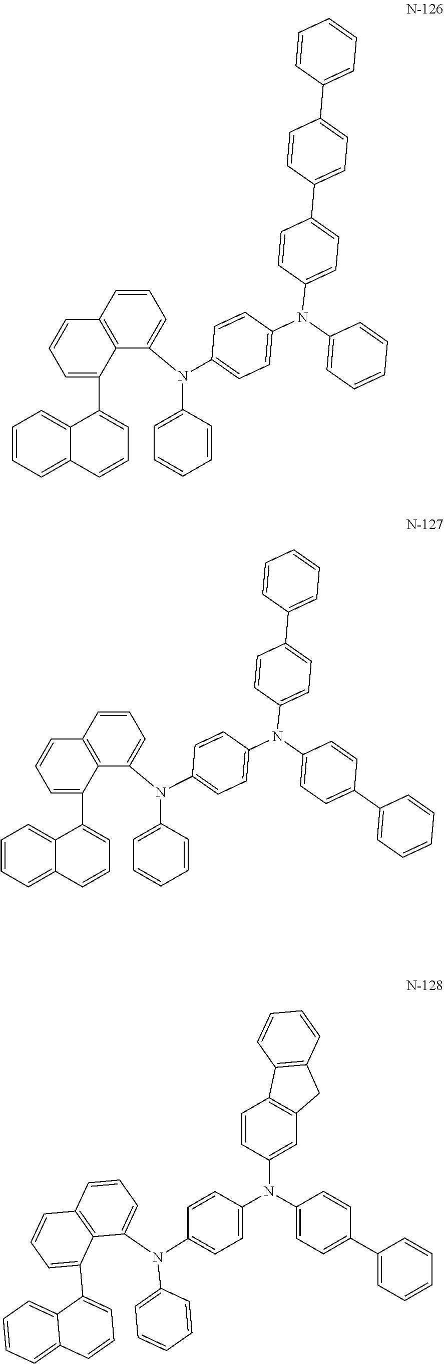 [SMILES: c1ccc(-c2ccc(-c3ccc(N(c4ccccc4)c4ccc(N(c5ccccc5)c5cccc6cccc(-c7cccc8ccccc78)c56)cc4)cc3)cc2)cc1 ; c1ccc(-c2ccc(N(c3ccc(-c4ccccc4)cc3)c3ccc(N(c4ccccc4)c4cccc5cccc(-c6cccc7ccccc67)c45)cc3)cc2)cc1 ; c1ccc(-c2ccc(N(c3ccc(N(c4ccccc4)c4cccc5cccc(-c6cccc7ccccc67)c45)cc3)c3ccc4c(c3)Cc3ccccc3-4)cc2)cc1]

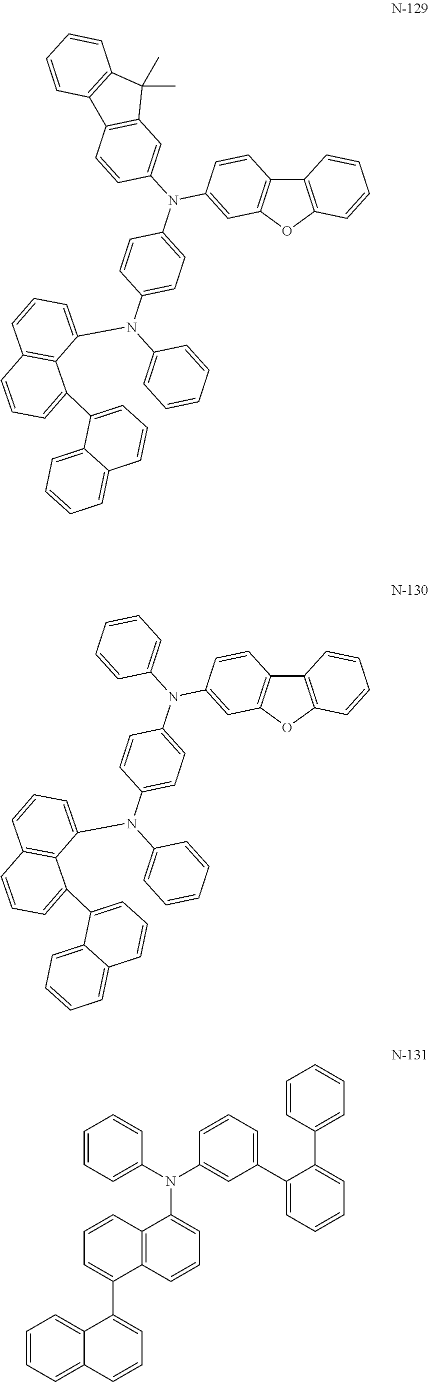 [SMILES: CC1(C)c2ccccc2-c2ccc(N(c3ccc(N(c4ccccc4)c4cccc5cccc(-c6cccc7ccccc67)c45)cc3)c3ccc4c(c3)oc3ccccc34)cc21 ; c1ccc(N(c2ccc(N(c3ccccc3)c3cccc4cccc(-c5cccc6ccccc56)c34)cc2)c2ccc3c(c2)oc2ccccc23)cc1 ; c1ccc(-c2ccccc2-c2cccc(N(c3ccccc3)c3cccc4c(-c5cccc6ccccc56)cccc34)c2)cc1]

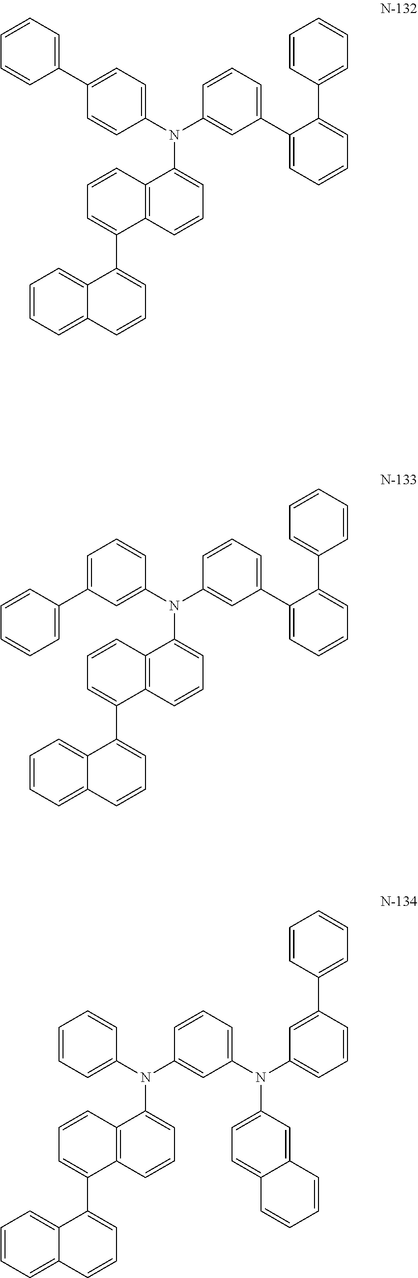 [SMILES: c1ccc(-c2ccc(N(c3cccc(-c4ccccc4-c4ccccc4)c3)c3cccc4c(-c5cccc6ccccc56)cccc34)cc2)cc1 ; c1ccc(-c2cccc(N(c3cccc(-c4ccccc4-c4ccccc4)c3)c3cccc4c(-c5cccc6ccccc56)cccc34)c2)cc1 ; c1ccc(-c2cccc(N(c3cccc(N(c4ccccc4)c4cccc5c(-c6cccc7ccccc67)cccc45)c3)c3ccc4ccccc4c3)c2)cc1]

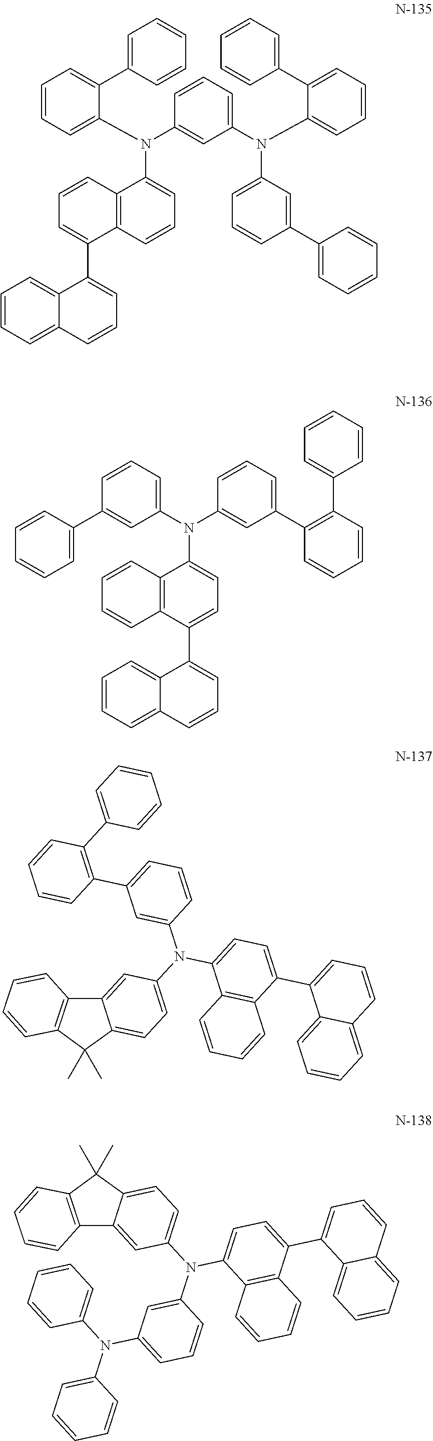

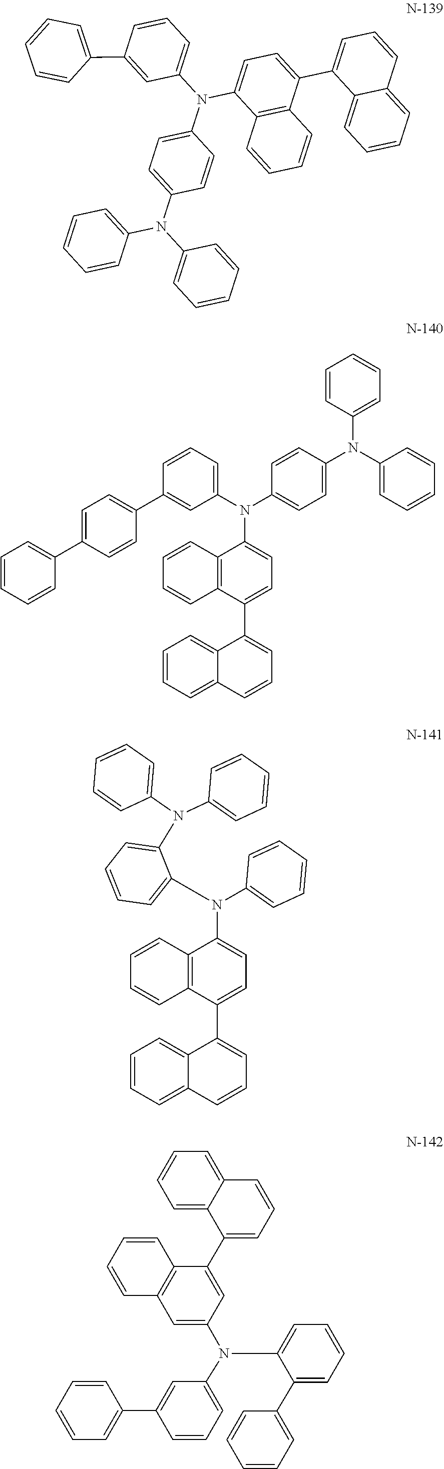 [SMILES: c1ccc(-c2cccc(N(c3ccc(N(c4ccccc4)c4ccccc4)cc3)c3ccc(-c4cccc5ccccc45)c4ccccc34)c2)cc1 ; c1ccc(-c2ccc(-c3cccc(N(c4ccc(N(c5ccccc5)c5ccccc5)cc4)c4ccc(-c5cccc6ccccc56)c5ccccc45)c3)cc2)cc1 ; c1ccc(N(c2ccccc2)c2ccccc2N(c2ccccc2)c2ccc(-c3cccc4ccccc34)c3ccccc23)cc1 ; c1ccc(-c2cccc(N(c3cc(-c4cccc5ccccc45)c4ccccc4c3)c3ccccc3-c3ccccc3)c2)cc1]

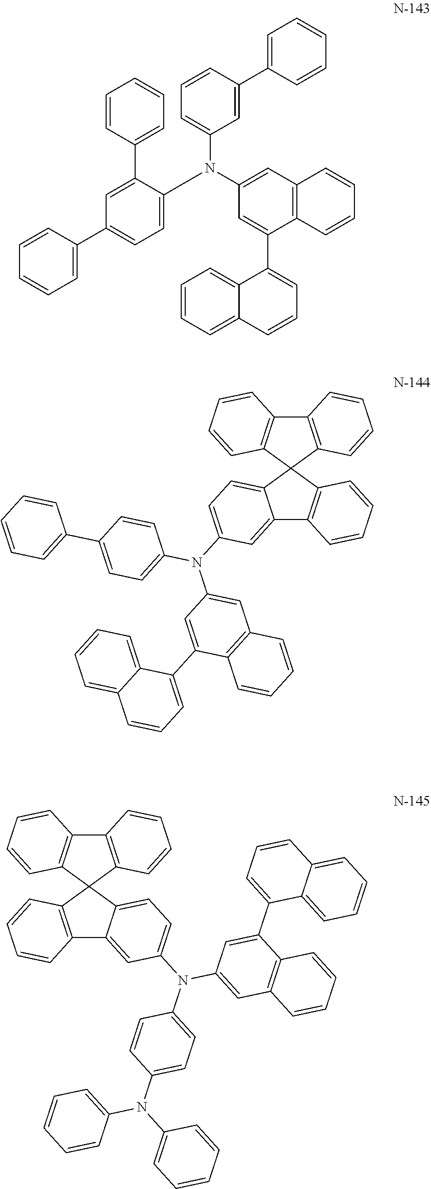 [SMILES: c1ccc(-c2cccc(N(c3cc(-c4cccc5ccccc45)c4ccccc4c3)c3ccc(-c4ccccc4)cc3-c3ccccc3)c2)cc1 ; c1ccc(-c2ccc(N(c3ccc4c(c3)-c3ccccc3C43c4ccccc4-c4ccccc43)c3cc(-c4cccc5ccccc45)c4ccccc4c3)cc2)cc1 ; c1ccc(N(c2ccccc2)c2ccc(N(c3ccc4c(c3)-c3ccccc3C43c4ccccc4-c4ccccc43)c3cc(-c4cccc5ccccc45)c4ccccc4c3)cc2)cc1]

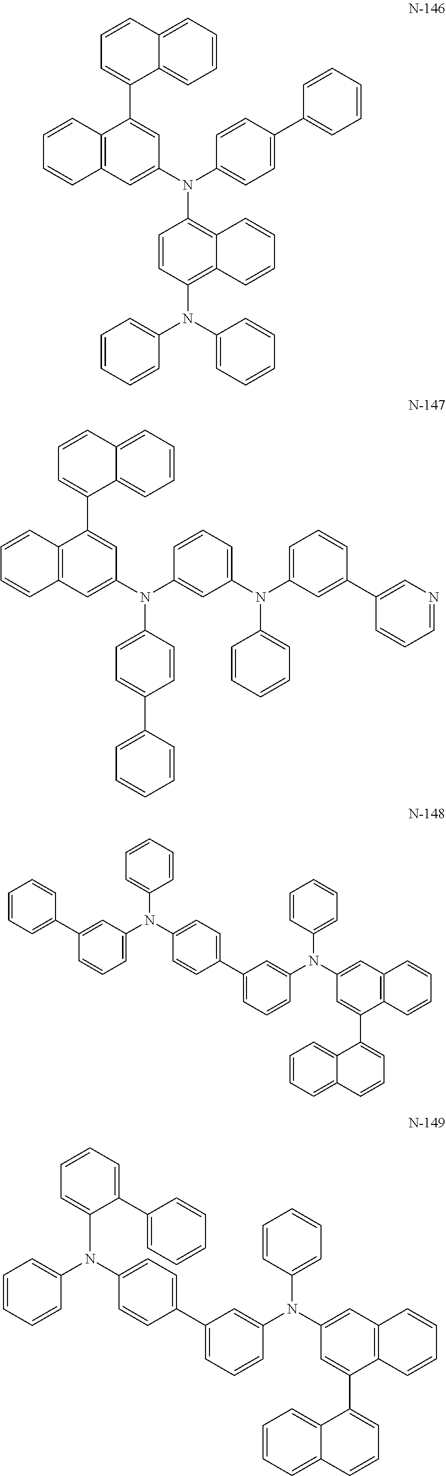

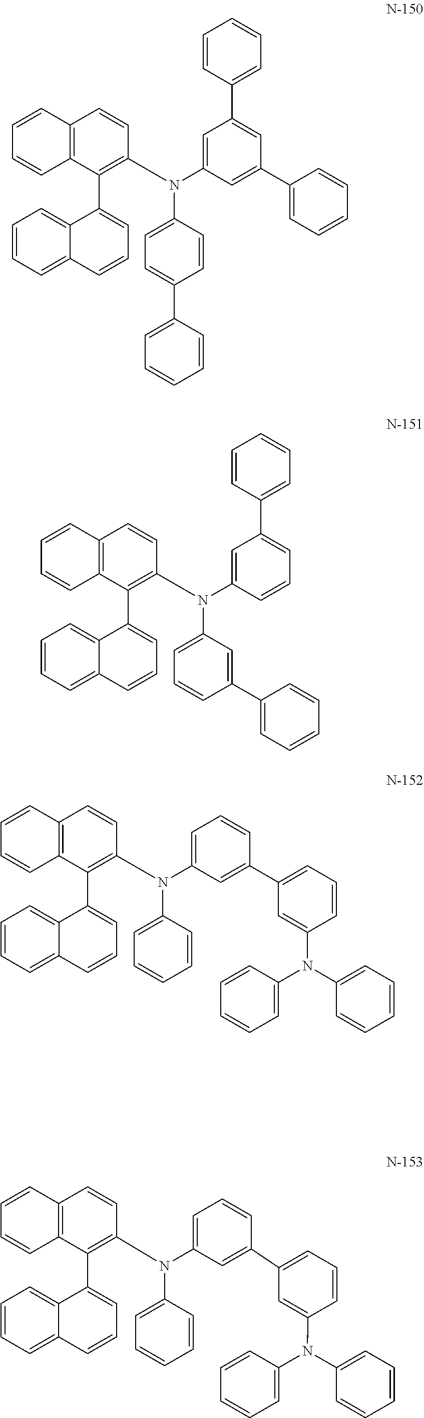 [SMILES: c1ccc(-c2ccc(N(c3cc(-c4ccccc4)cc(-c4ccccc4)c3)c3ccc4ccccc4c3-c3cccc4ccccc34)cc2)cc1 ; c1ccc(-c2cccc(N(c3cccc(-c4ccccc4)c3)c3ccc4ccccc4c3-c3cccc4ccccc34)c2)cc1 ; c1ccc(N(c2ccccc2)c2cccc(-c3cccc(N(c4ccccc4)c4ccc5ccccc5c4-c4cccc5ccccc45)c3)c2)cc1 ; c1ccc(N(c2ccccc2)c2cccc(-c3cccc(N(c4ccccc4)c4ccc5ccccc5c4-c4cccc5ccccc45)c3)c2)cc1]

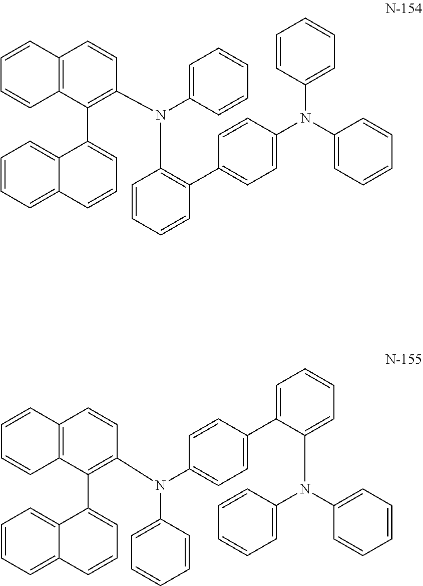 [SMILES: c1ccc(N(c2ccccc2)c2ccc(-c3ccccc3N(c3ccccc3)c3ccc4ccccc4c3-c3cccc4ccccc34)cc2)cc1 ; c1ccc(N(c2ccccc2)c2ccccc2-c2ccc(N(c3ccccc3)c3ccc4ccccc4c3-c3cccc4ccccc34)cc2)cc1]

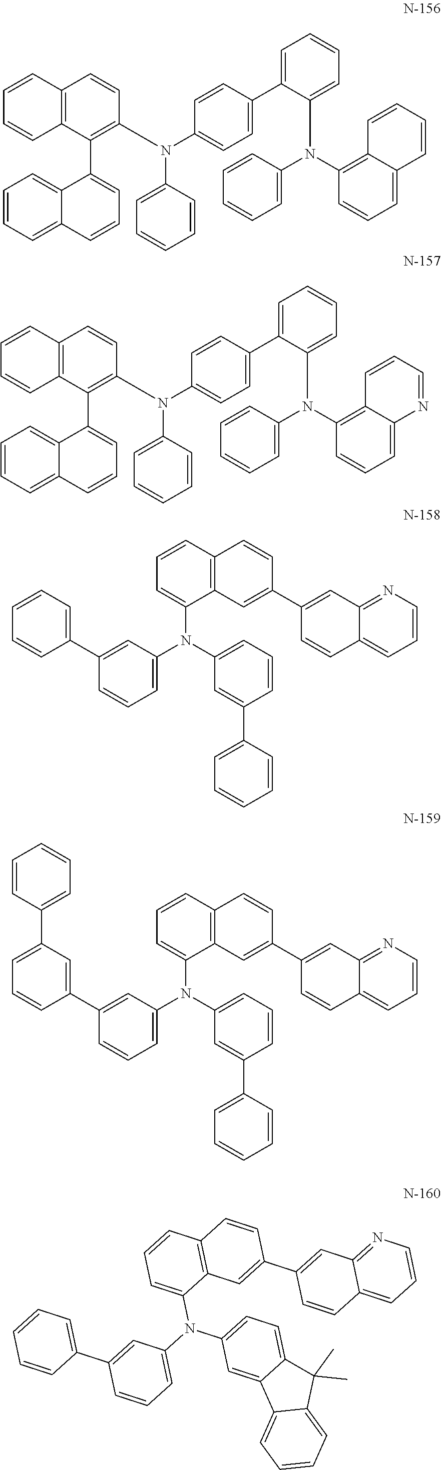

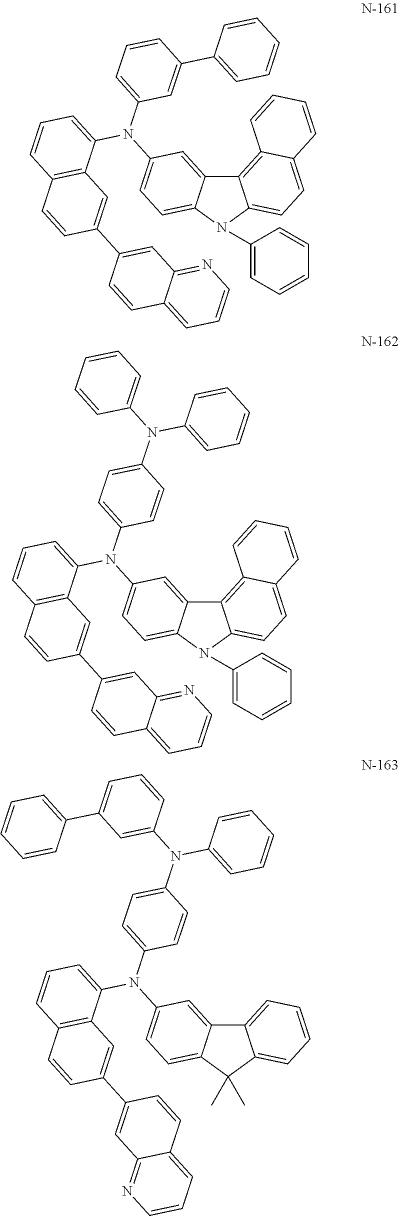

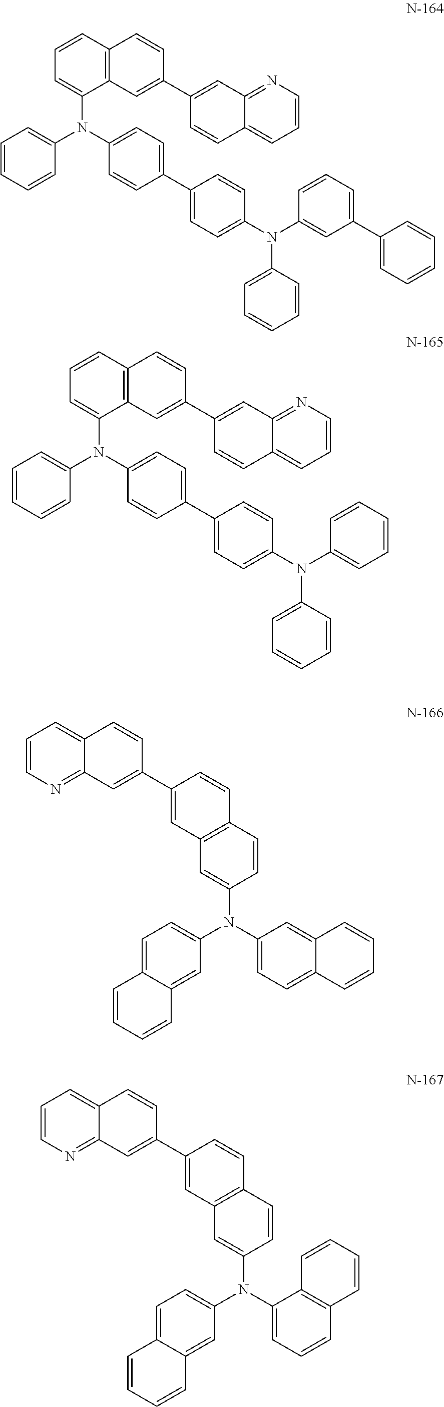 [SMILES: c1ccc(-c2cccc(N(c3ccccc3)c3ccc(-c4ccc(N(c5ccccc5)c5cccc6ccc(-c7ccc8cccnc8c7)cc56)cc4)cc3)c2)cc1 ; c1ccc(N(c2ccccc2)c2ccc(-c3ccc(N(c4ccccc4)c4cccc5ccc(-c6ccc7cccnc7c6)cc45)cc3)cc2)cc1 ; c1ccc2cc(N(c3ccc4ccccc4c3)c3ccc4ccc(-c5ccc6cccnc6c5)cc4c3)ccc2c1 ; c1ccc2cc(N(c3ccc4ccc(-c5ccc6cccnc6c5)cc4c3)c3cccc4ccccc34)ccc2c1]

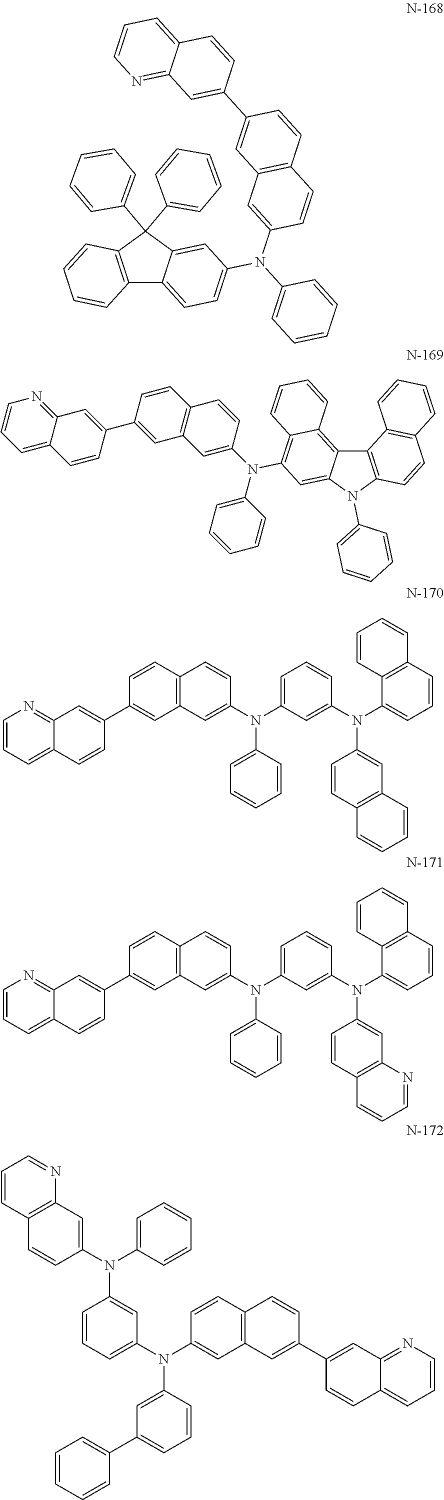

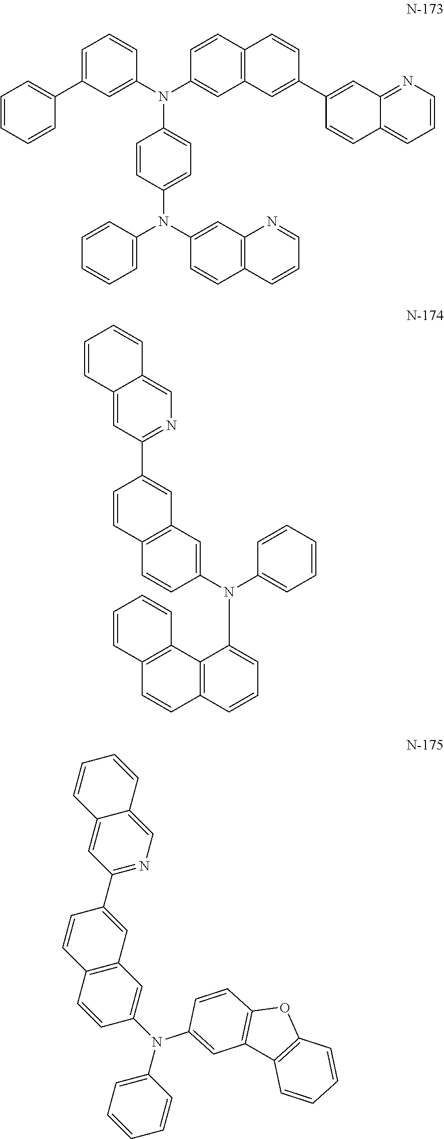

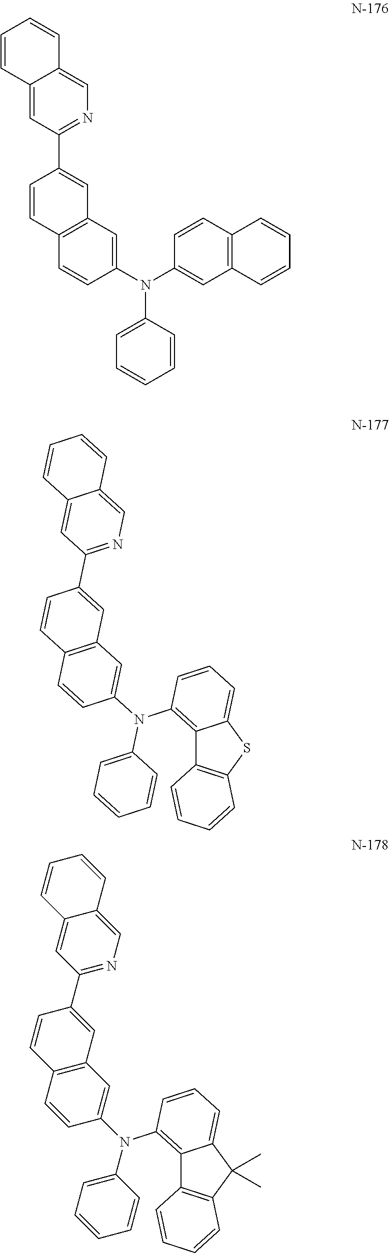 [SMILES: c1ccc(N(c2ccc3ccccc3c2)c2ccc3ccc(-c4cc5ccccc5cn4)cc3c2)cc1 ; c1ccc(N(c2ccc3ccc(-c4cc5ccccc5cn4)cc3c2)c2cccc3sc4ccccc4c23)cc1 ; CC1(C)c2ccccc2-c2c(N(c3ccccc3)c3ccc4ccc(-c5cc6ccccc6cn5)cc4c3)cccc21]

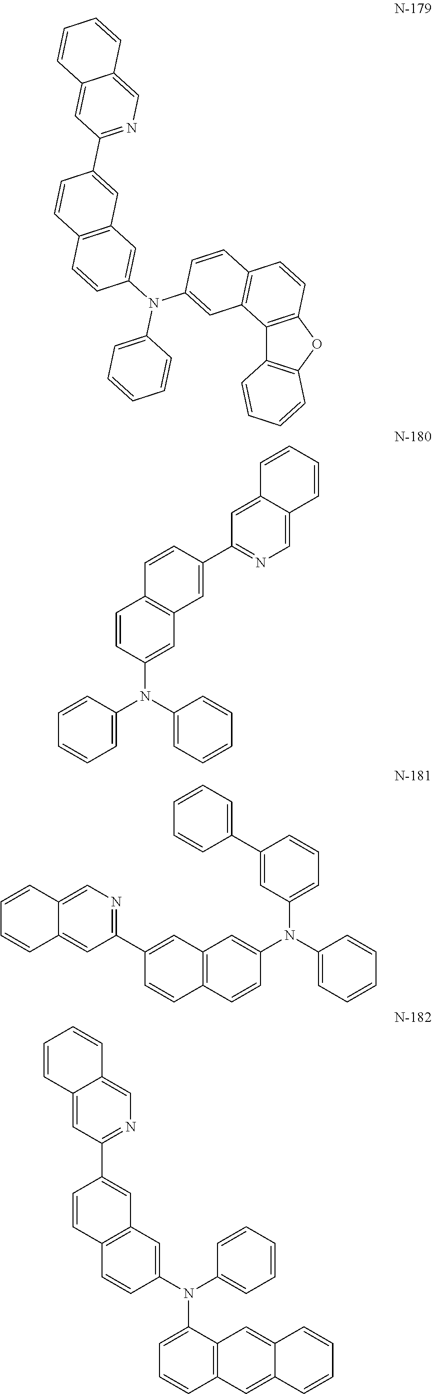

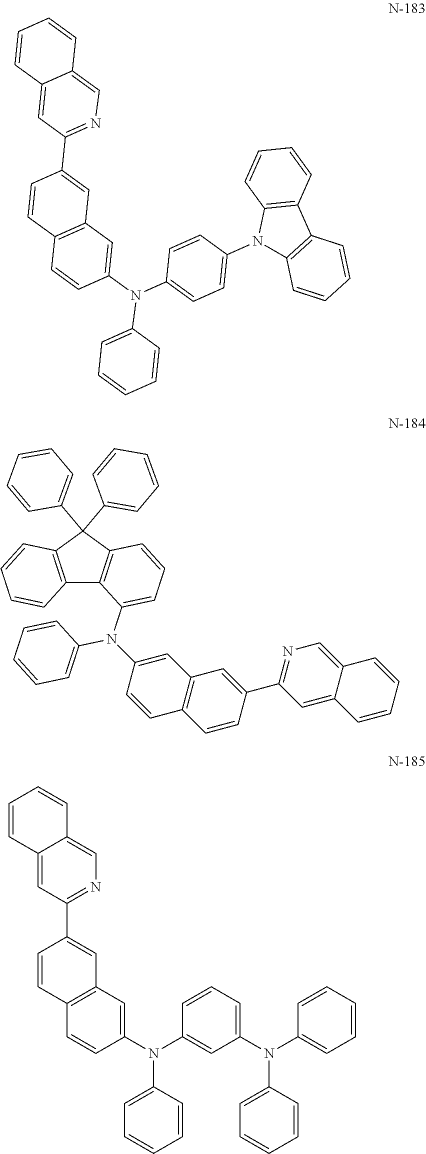 [SMILES: c1ccc(N(c2ccc(-n3c4ccccc4c4ccccc43)cc2)c2ccc3ccc(-c4cc5ccccc5cn4)cc3c2)cc1 ; c1ccc(N(c2ccc3ccc(-c4cc5ccccc5cn4)cc3c2)c2cccc3c2-c2ccccc2C3(c2ccccc2)c2ccccc2)cc1 ; c1ccc(N(c2ccccc2)c2cccc(N(c3ccccc3)c3ccc4ccc(-c5cc6ccccc6cn5)cc4c3)c2)cc1]

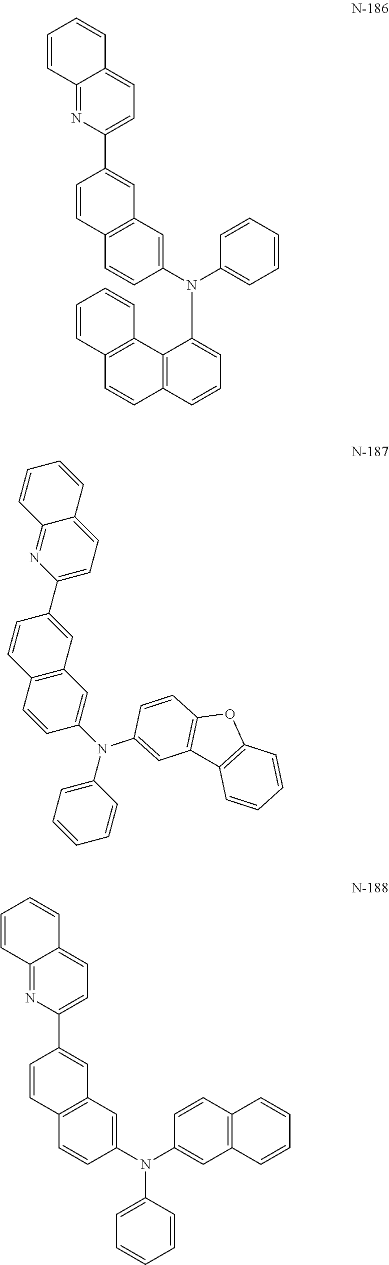 [SMILES: c1ccc(N(c2ccc3ccc(-c4ccc5ccccc5n4)cc3c2)c2cccc3ccc4ccccc4c23)cc1 ; c1ccc(N(c2ccc3ccc(-c4ccc5ccccc5n4)cc3c2)c2ccc3oc4ccccc4c3c2)cc1 ; c1ccc(N(c2ccc3ccccc3c2)c2ccc3ccc(-c4ccc5ccccc5n4)cc3c2)cc1]

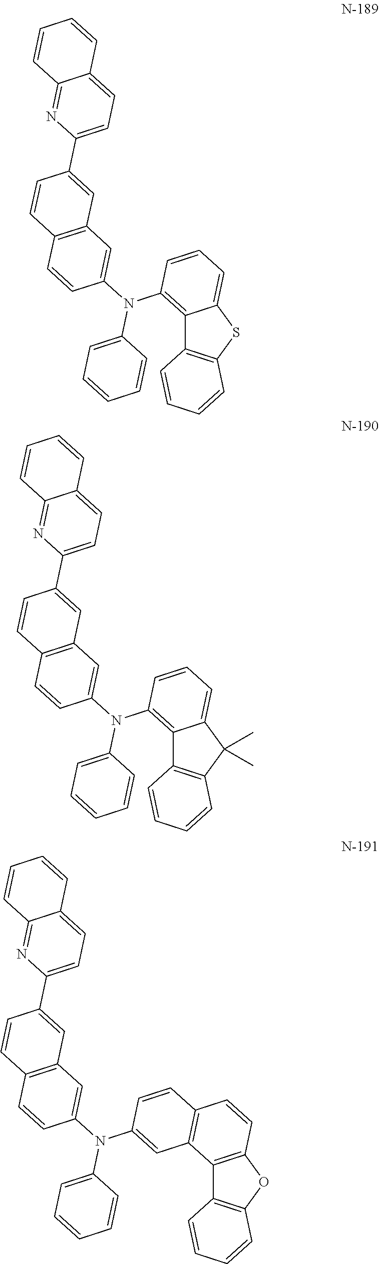 [SMILES: c1ccc(N(c2ccc3ccc(-c4ccc5ccccc5n4)cc3c2)c2cccc3sc4ccccc4c23)cc1 ; CC1(C)c2ccccc2-c2c(N(c3ccccc3)c3ccc4ccc(-c5ccc6ccccc6n5)cc4c3)cccc21 ; c1ccc(N(c2ccc3ccc(-c4ccc5ccccc5n4)cc3c2)c2ccc3ccc4oc5ccccc5c4c3c2)cc1]

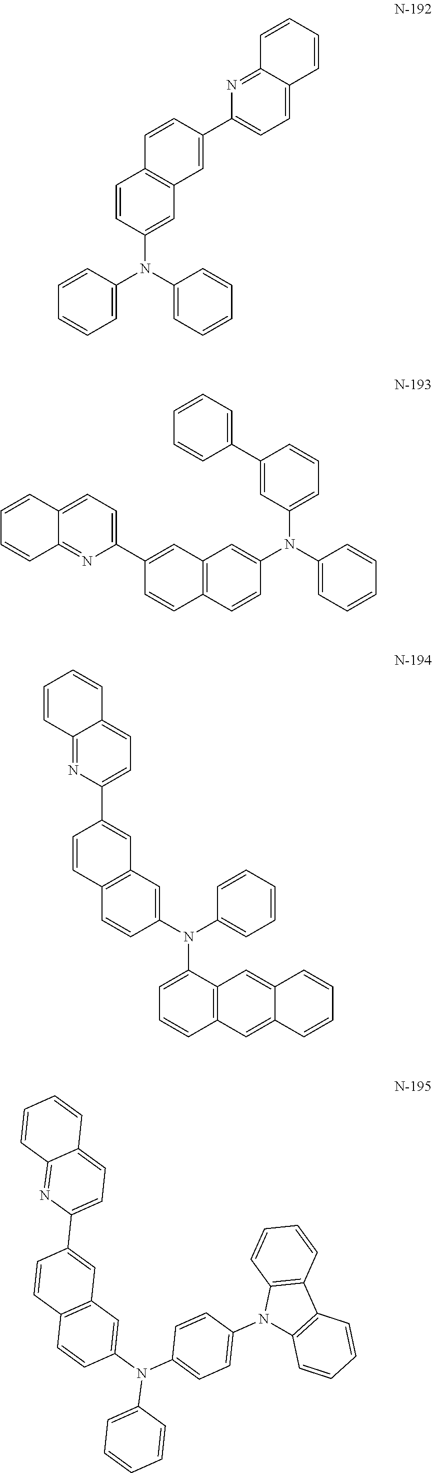 [SMILES: c1ccc(N(c2ccccc2)c2ccc3ccc(-c4ccc5ccccc5n4)cc3c2)cc1 ; c1ccc(-c2cccc(N(c3ccccc3)c3ccc4ccc(-c5ccc6ccccc6n5)cc4c3)c2)cc1 ; c1ccc(N(c2ccc3ccc(-c4ccc5ccccc5n4)cc3c2)c2cccc3cc4ccccc4cc23)cc1 ; c1ccc(N(c2ccc(-n3c4ccccc4c4ccccc43)cc2)c2ccc3ccc(-c4ccc5ccccc5n4)cc3c2)cc1]

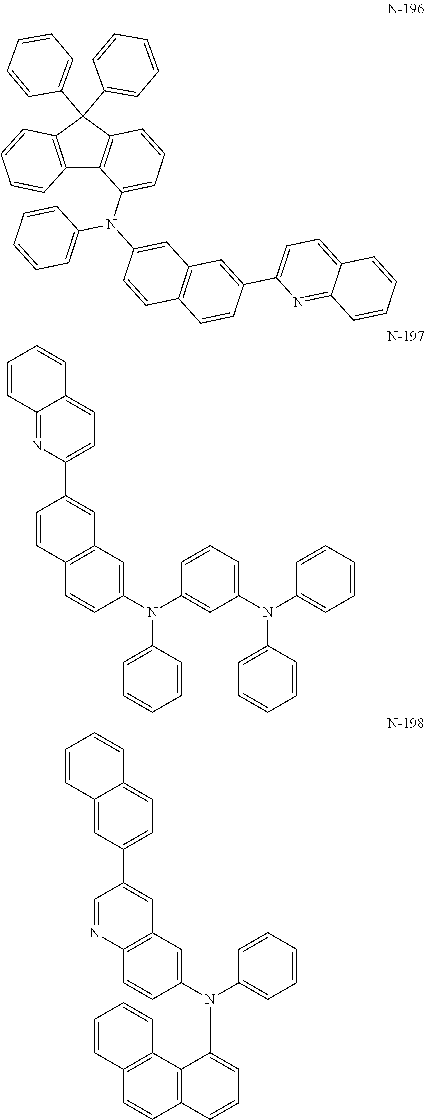 [SMILES: c1ccc(N(c2ccc3ccc(-c4ccc5ccccc5n4)cc3c2)c2cccc3c2-c2ccccc2C3(c2ccccc2)c2ccccc2)cc1 ; c1ccc(N(c2ccccc2)c2cccc(N(c3ccccc3)c3ccc4ccc(-c5ccc6ccccc6n5)cc4c3)c2)cc1 ; c1ccc(N(c2ccc3ncc(-c4ccc5ccccc5c4)cc3c2)c2cccc3ccc4ccccc4c23)cc1]

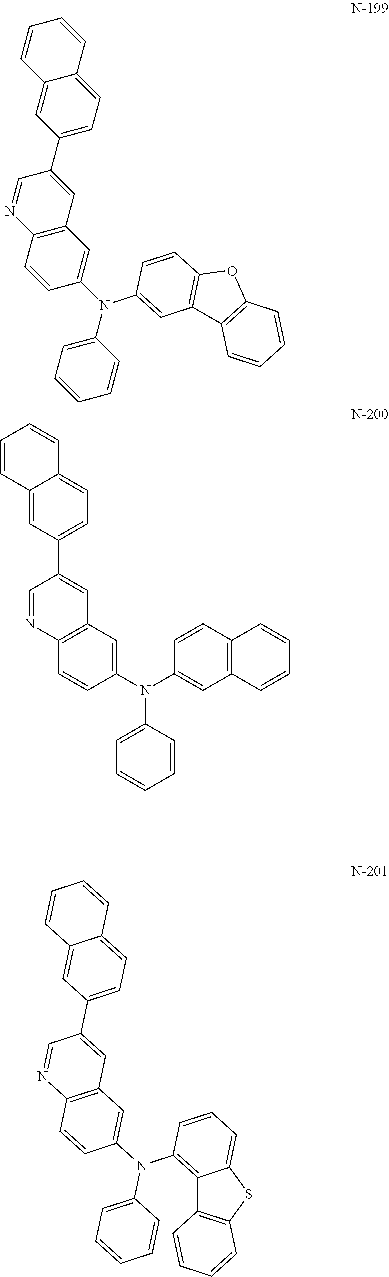

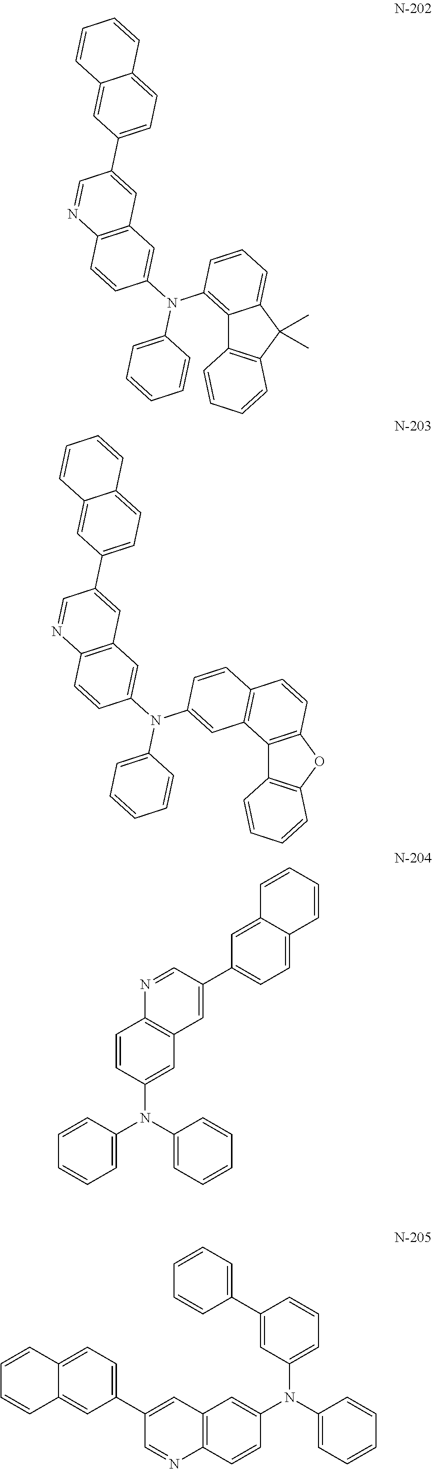

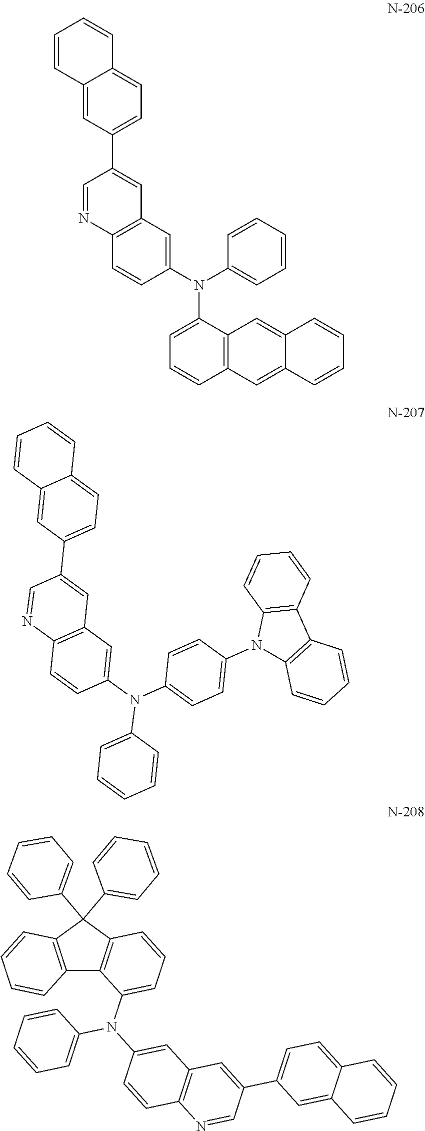

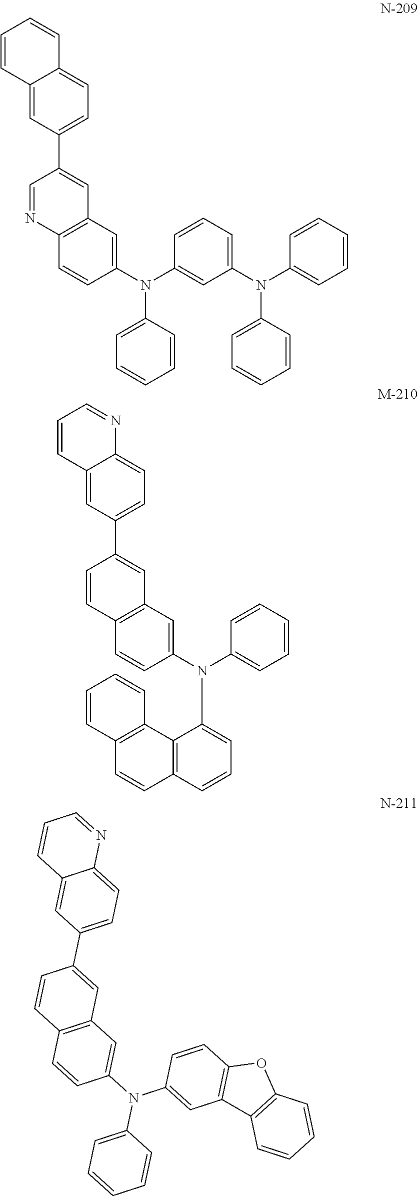

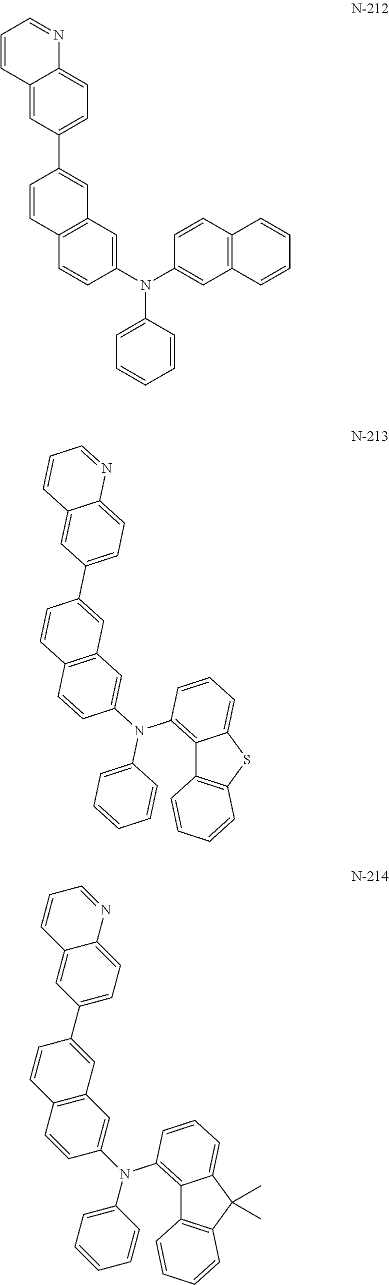 [SMILES: c1ccc(N(c2ccc3ccccc3c2)c2ccc3ccc(-c4ccc5ncccc5c4)cc3c2)cc1 ; c1ccc(N(c2ccc3ccc(-c4ccc5ncccc5c4)cc3c2)c2cccc3sc4ccccc4c23)cc1 ; CC1(C)c2ccccc2-c2c(N(c3ccccc3)c3ccc4ccc(-c5ccc6ncccc6c5)cc4c3)cccc21]

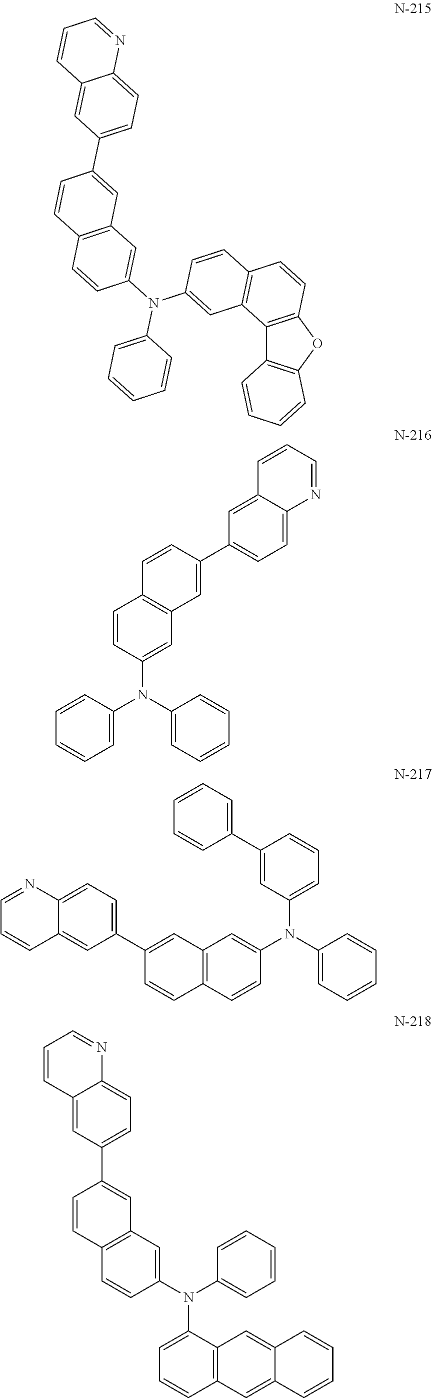

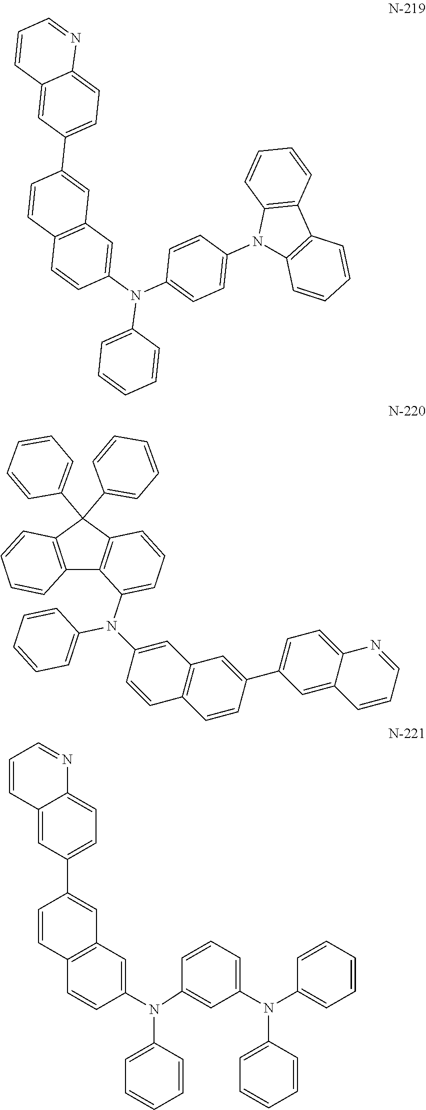

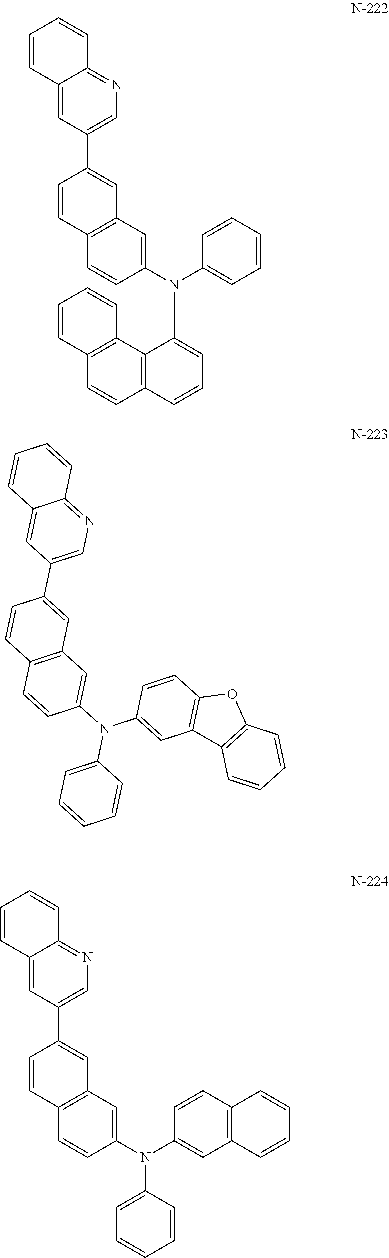 [SMILES: c1ccc(N(c2ccc3ccc(-c4cnc5ccccc5c4)cc3c2)c2cccc3ccc4ccccc4c23)cc1 ; c1ccc(N(c2ccc3ccc(-c4cnc5ccccc5c4)cc3c2)c2ccc3oc4ccccc4c3c2)cc1 ; c1ccc(N(c2ccc3ccccc3c2)c2ccc3ccc(-c4cnc5ccccc5c4)cc3c2)cc1]

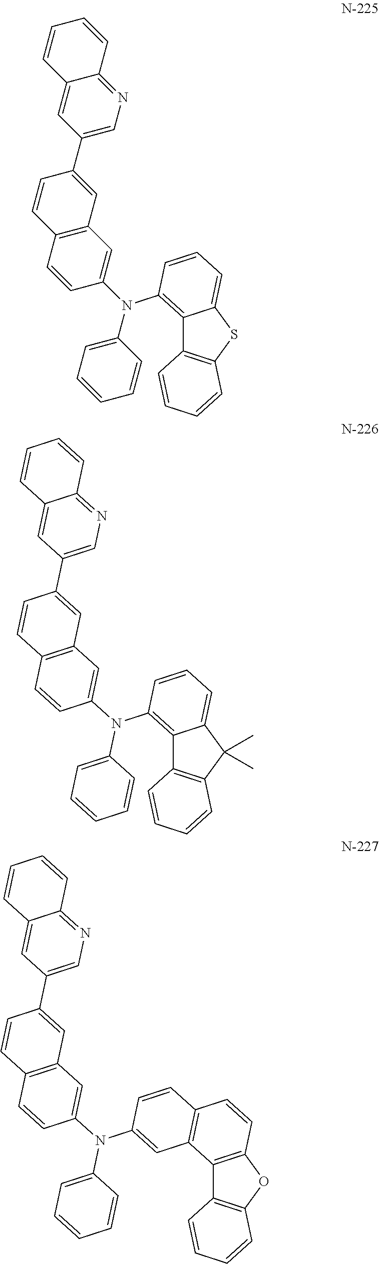 [SMILES: c1ccc(N(c2ccc3ccc(-c4cnc5ccccc5c4)cc3c2)c2cccc3sc4ccccc4c23)cc1 ; CC1(C)c2ccccc2-c2c(N(c3ccccc3)c3ccc4ccc(-c5cnc6ccccc6c5)cc4c3)cccc21 ; c1ccc(N(c2ccc3ccc(-c4cnc5ccccc5c4)cc3c2)c2ccc3ccc4oc5ccccc5c4c3c2)cc1]

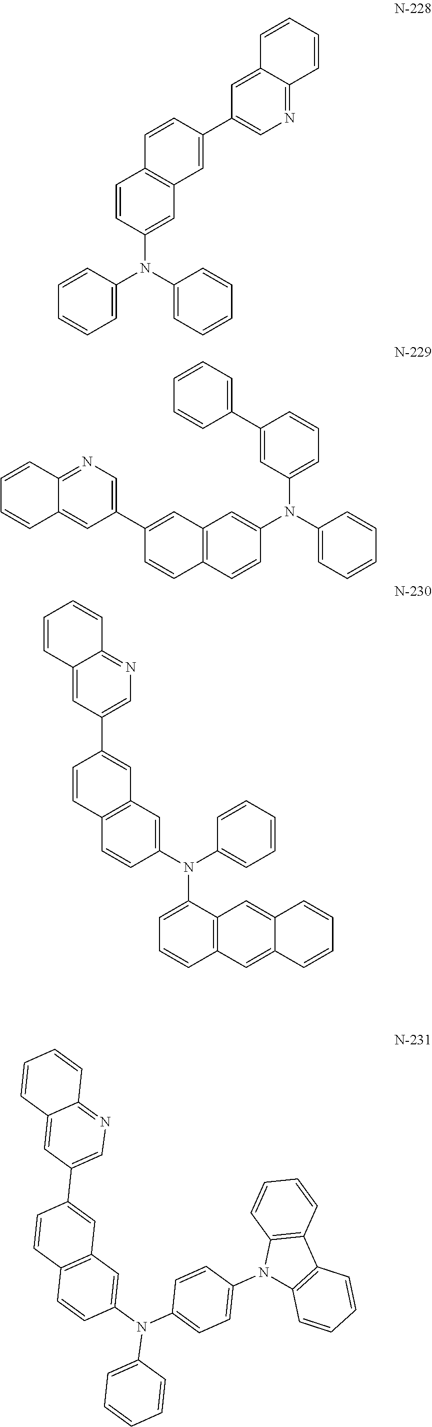

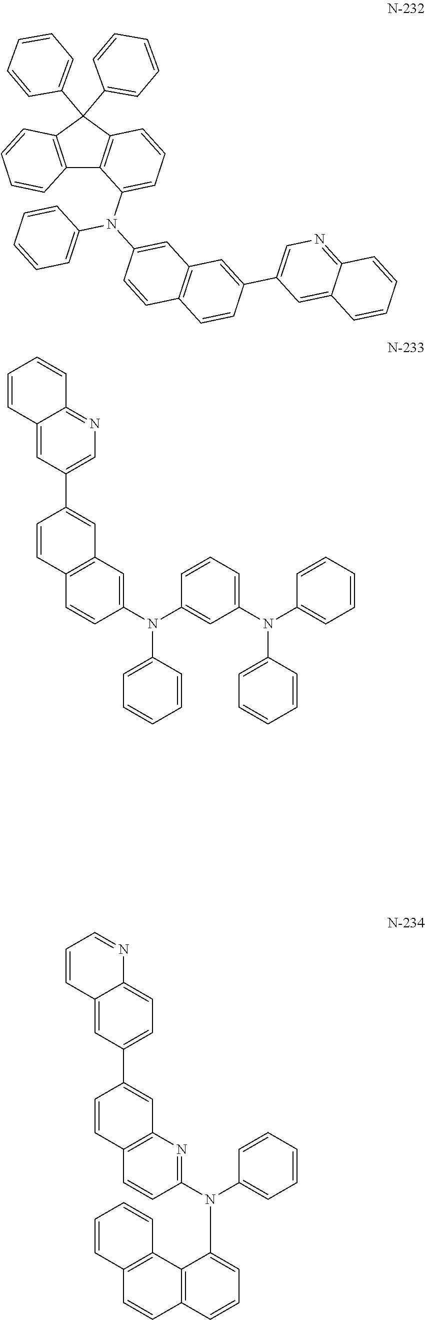 [SMILES: c1ccc(N(c2ccc3ccc(-c4cnc5ccccc5c4)cc3c2)c2cccc3c2-c2ccccc2C3(c2ccccc2)c2ccccc2)cc1 ; c1ccc(N(c2ccccc2)c2cccc(N(c3ccccc3)c3ccc4ccc(-c5cnc6ccccc6c5)cc4c3)c2)cc1 ; c1ccc(N(c2ccc3ccc(-c4ccc5ncccc5c4)cc3n2)c2cccc3ccc4ccccc4c23)cc1]

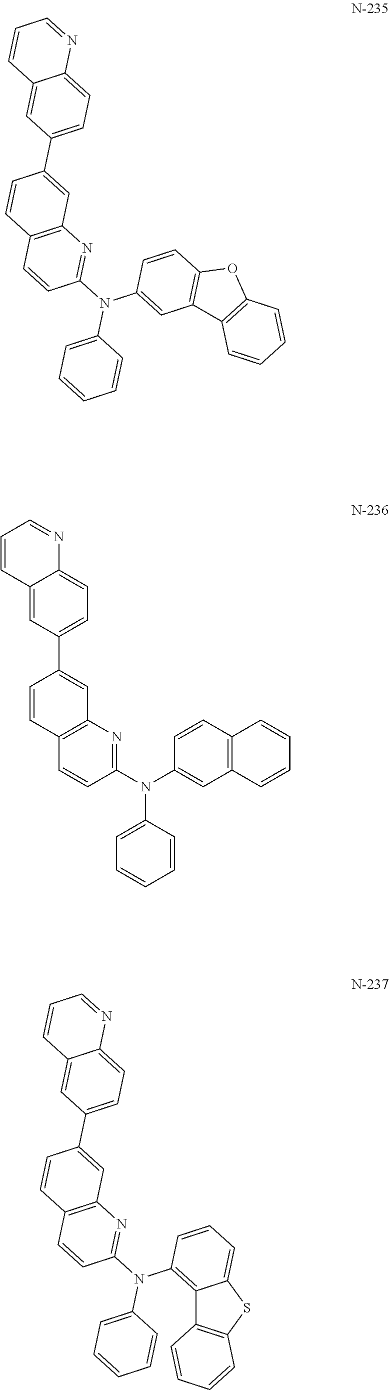 [SMILES: c1ccc(N(c2ccc3oc4ccccc4c3c2)c2ccc3ccc(-c4ccc5ncccc5c4)cc3n2)cc1 ; c1ccc(N(c2ccc3ccccc3c2)c2ccc3ccc(-c4ccc5ncccc5c4)cc3n2)cc1 ; c1ccc(N(c2ccc3ccc(-c4ccc5ncccc5c4)cc3n2)c2cccc3sc4ccccc4c23)cc1]

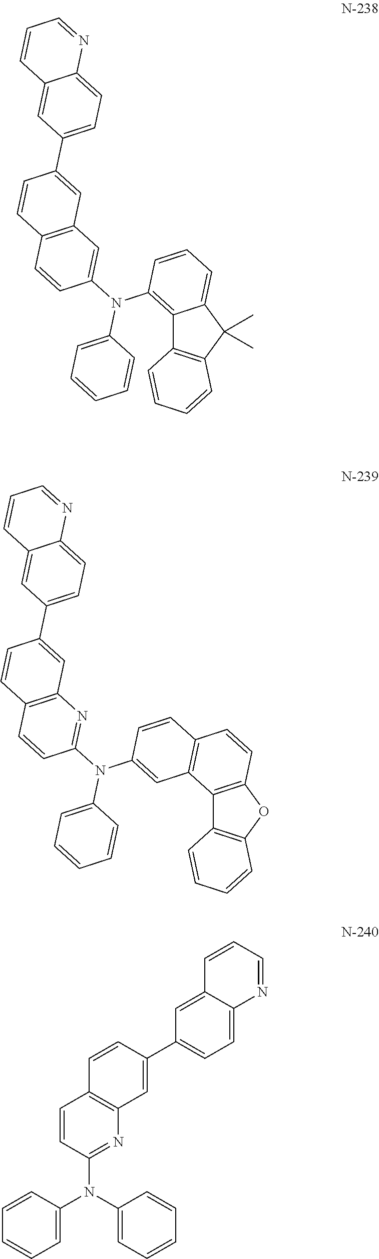 [SMILES: CC1(C)c2ccccc2-c2c(N(c3ccccc3)c3ccc4ccc(-c5ccc6ncccc6c5)cc4c3)cccc21 ; c1ccc(N(c2ccc3ccc4oc5ccccc5c4c3c2)c2ccc3ccc(-c4ccc5ncccc5c4)cc3n2)cc1 ; c1ccc(N(c2ccccc2)c2ccc3ccc(-c4ccc5ncccc5c4)cc3n2)cc1]

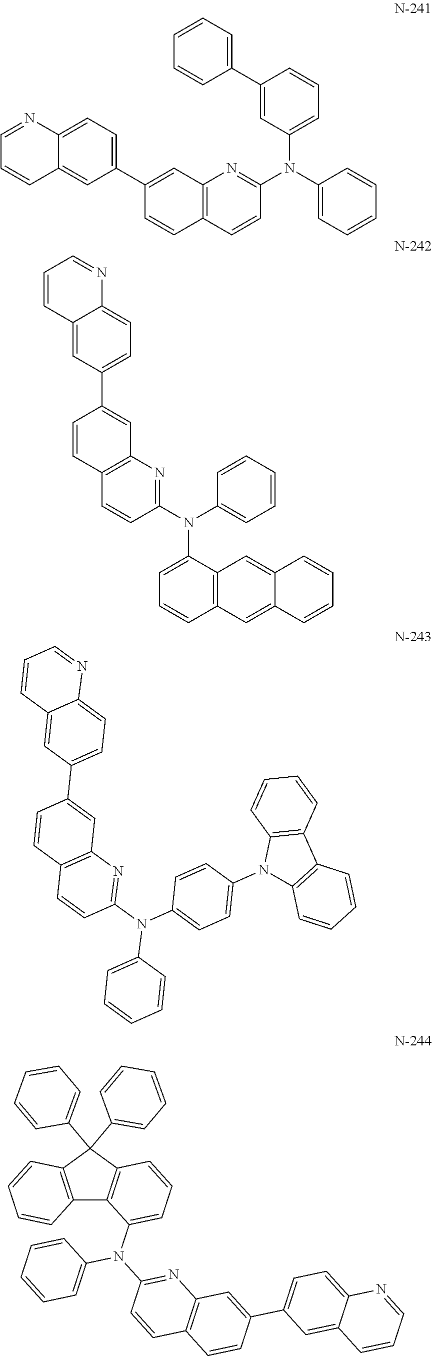

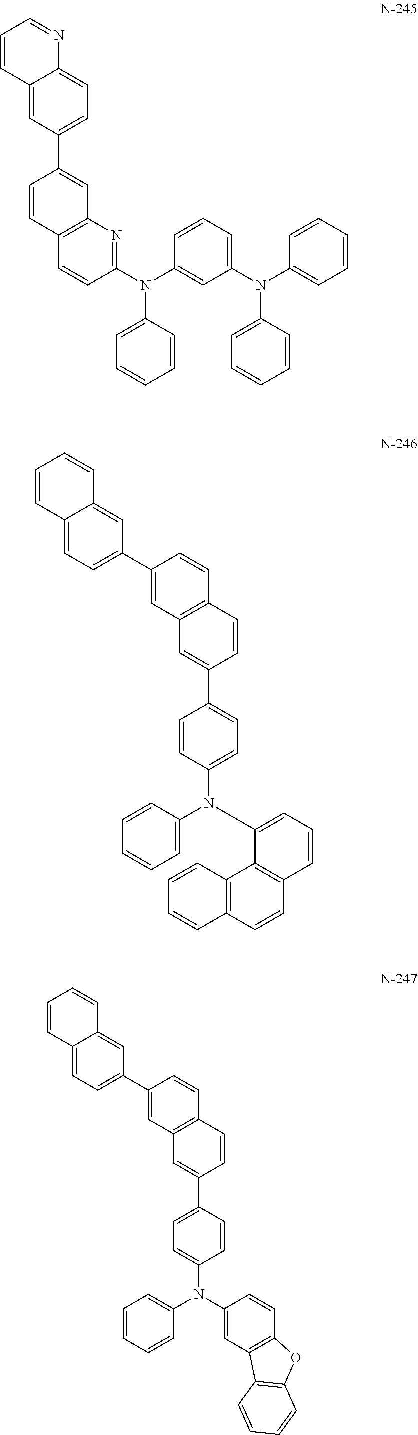 [SMILES: c1ccc(N(c2ccccc2)c2cccc(N(c3ccccc3)c3ccc4ccc(-c5ccc6ncccc6c5)cc4n3)c2)cc1 ; c1ccc(N(c2ccc(-c3ccc4ccc(-c5ccc6ccccc6c5)cc4c3)cc2)c2cccc3ccc4ccccc4c23)cc1 ; c1ccc(N(c2ccc(-c3ccc4ccc(-c5ccc6ccccc6c5)cc4c3)cc2)c2ccc3oc4ccccc4c3c2)cc1]

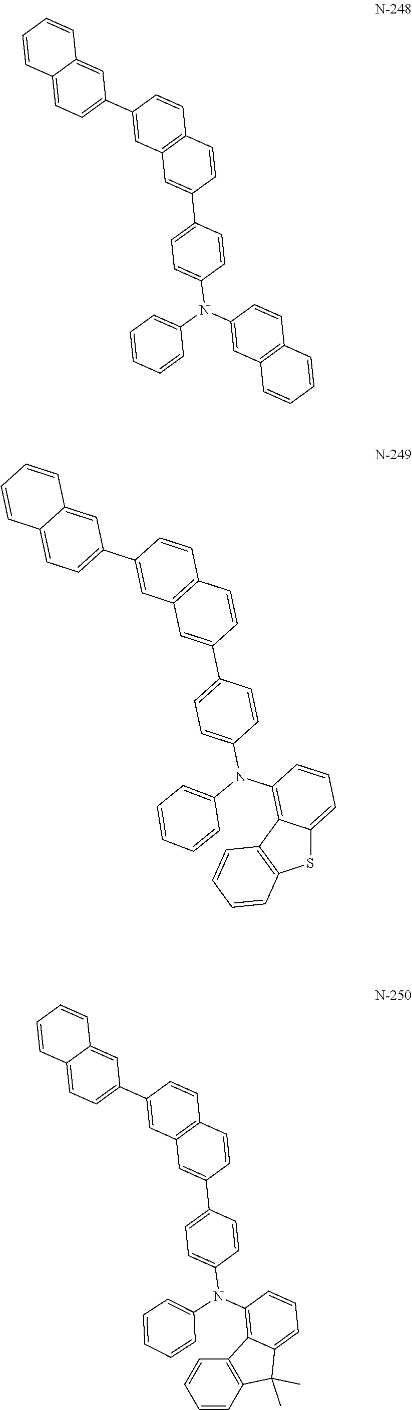 [SMILES: c1ccc(N(c2ccc(-c3ccc4ccc(-c5ccc6ccccc6c5)cc4c3)cc2)c2ccc3ccccc3c2)cc1 ; c1ccc(N(c2ccc(-c3ccc4ccc(-c5ccc6ccccc6c5)cc4c3)cc2)c2cccc3sc4ccccc4c23)cc1 ; CC1(C)c2ccccc2-c2c(N(c3ccccc3)c3ccc(-c4ccc5ccc(-c6ccc7ccccc7c6)cc5c4)cc3)cccc21]

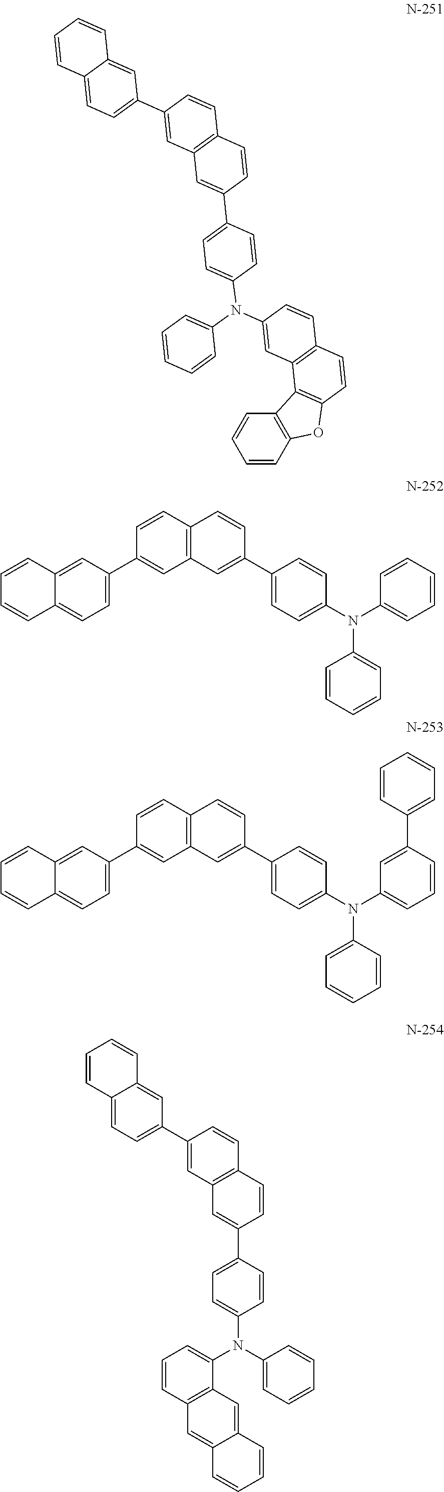 [SMILES: c1ccc(N(c2ccc(-c3ccc4ccc(-c5ccc6ccccc6c5)cc4c3)cc2)c2ccc3ccc4oc5ccccc5c4c3c2)cc1 ; c1ccc(N(c2ccccc2)c2ccc(-c3ccc4ccc(-c5ccc6ccccc6c5)cc4c3)cc2)cc1 ; c1ccc(-c2cccc(N(c3ccccc3)c3ccc(-c4ccc5ccc(-c6ccc7ccccc7c6)cc5c4)cc3)c2)cc1 ; c1ccc(N(c2ccc(-c3ccc4ccc(-c5ccc6ccccc6c5)cc4c3)cc2)c2cccc3cc4ccccc4cc23)cc1]

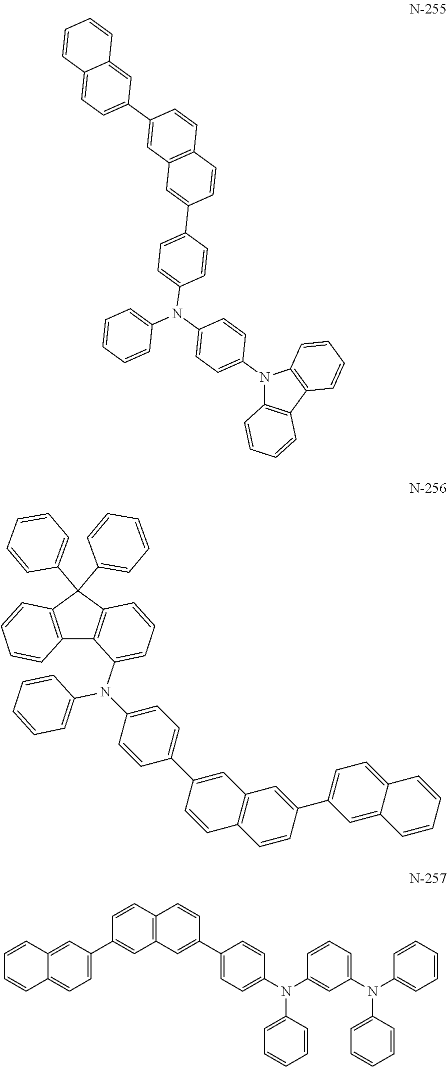 [SMILES: c1ccc(N(c2ccc(-c3ccc4ccc(-c5ccc6ccccc6c5)cc4c3)cc2)c2ccc(-n3c4ccccc4c4ccccc43)cc2)cc1 ; c1ccc(N(c2ccc(-c3ccc4ccc(-c5ccc6ccccc6c5)cc4c3)cc2)c2cccc3c2-c2ccccc2C3(c2ccccc2)c2ccccc2)cc1 ; c1ccc(N(c2ccccc2)c2cccc(N(c3ccccc3)c3ccc(-c4ccc5ccc(-c6ccc7ccccc7c6)cc5c4)cc3)c2)cc1]

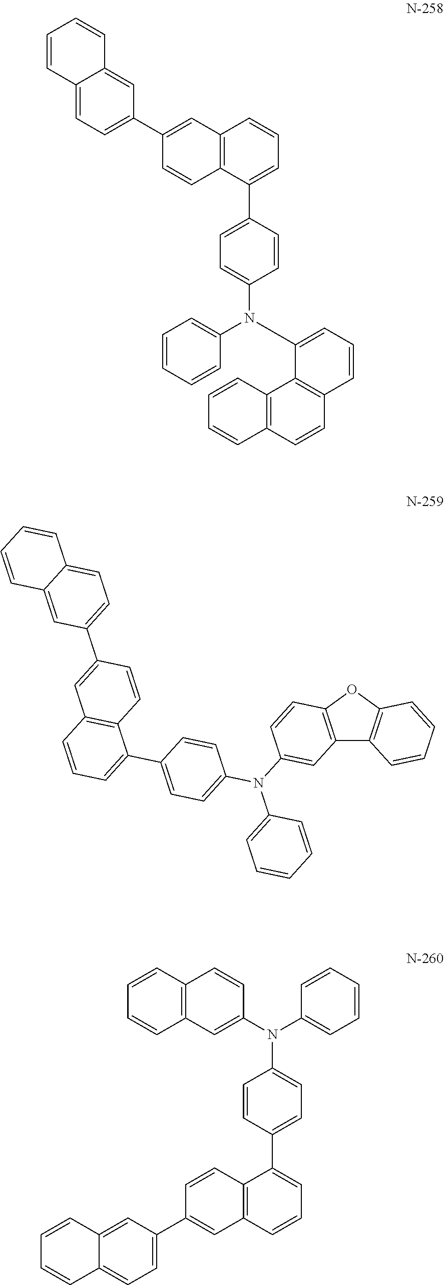 [SMILES: c1ccc(N(c2ccc(-c3cccc4cc(-c5ccc6ccccc6c5)ccc34)cc2)c2cccc3ccc4ccccc4c23)cc1 ; c1ccc(N(c2ccc(-c3cccc4cc(-c5ccc6ccccc6c5)ccc34)cc2)c2ccc3oc4ccccc4c3c2)cc1 ; c1ccc(N(c2ccc(-c3cccc4cc(-c5ccc6ccccc6c5)ccc34)cc2)c2ccc3ccccc3c2)cc1]

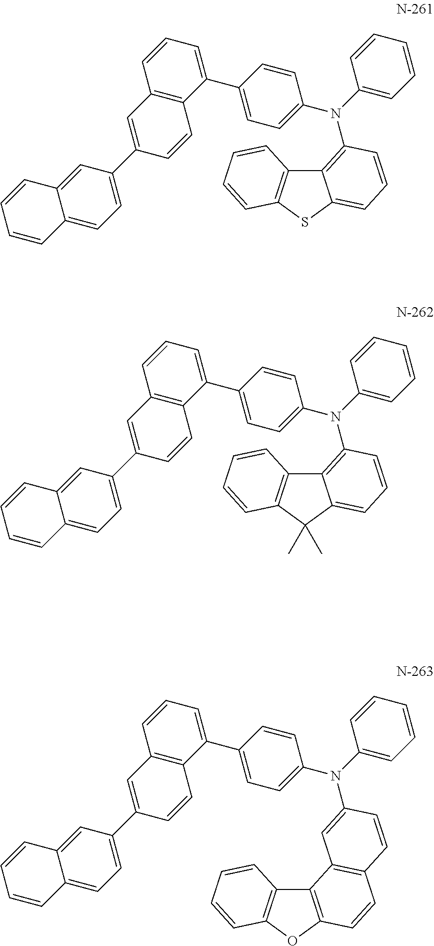 [SMILES: c1ccc(N(c2ccc(-c3cccc4cc(-c5ccc6ccccc6c5)ccc34)cc2)c2cccc3sc4ccccc4c23)cc1 ; CC1(C)c2ccccc2-c2c(N(c3ccccc3)c3ccc(-c4cccc5cc(-c6ccc7ccccc7c6)ccc45)cc3)cccc21 ; c1ccc(N(c2ccc(-c3cccc4cc(-c5ccc6ccccc6c5)ccc34)cc2)c2ccc3ccc4oc5ccccc5c4c3c2)cc1]

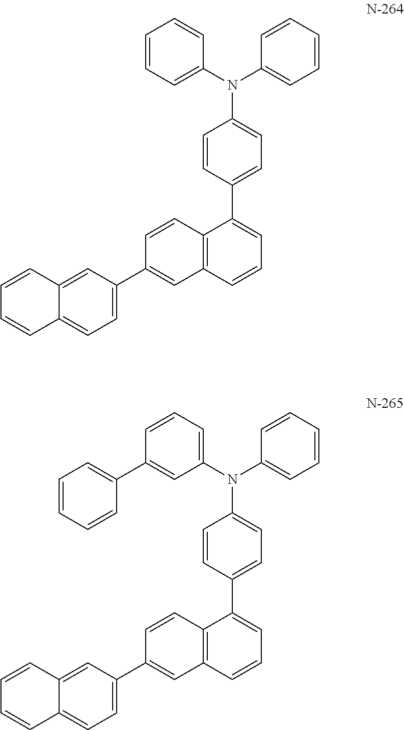 [SMILES: c1ccc(N(c2ccccc2)c2ccc(-c3cccc4cc(-c5ccc6ccccc6c5)ccc34)cc2)cc1 ; c1ccc(-c2cccc(N(c3ccccc3)c3ccc(-c4cccc5cc(-c6ccc7ccccc7c6)ccc45)cc3)c2)cc1]

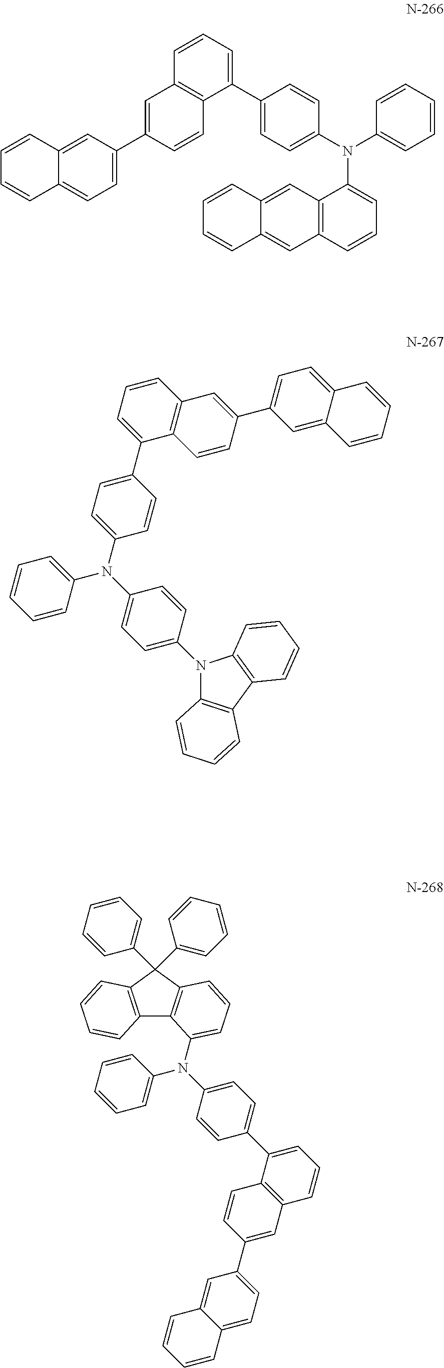 [SMILES: c1ccc(N(c2ccc(-c3cccc4cc(-c5ccc6ccccc6c5)ccc34)cc2)c2cccc3cc4ccccc4cc23)cc1 ; c1ccc(N(c2ccc(-c3cccc4cc(-c5ccc6ccccc6c5)ccc34)cc2)c2ccc(-n3c4ccccc4c4ccccc43)cc2)cc1 ; c1ccc(N(c2ccc(-c3cccc4cc(-c5ccc6ccccc6c5)ccc34)cc2)c2cccc3c2-c2ccccc2C3(c2ccccc2)c2ccccc2)cc1]

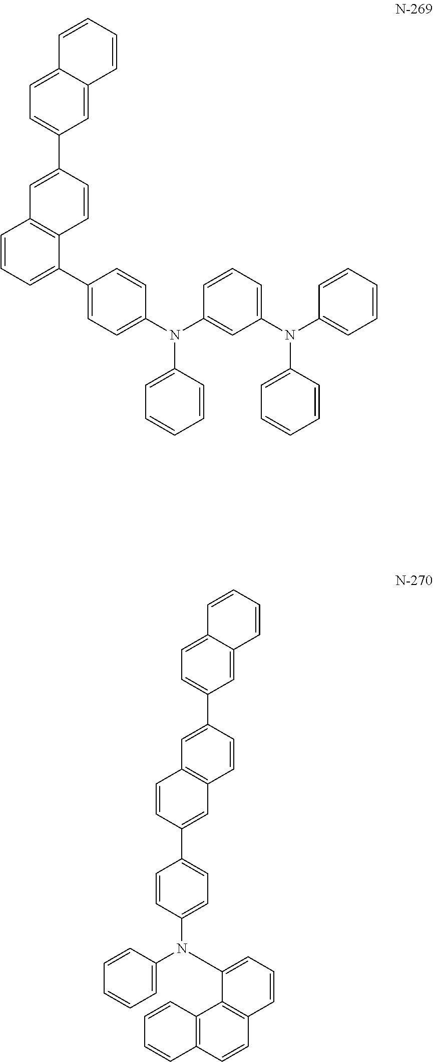 [SMILES: c1ccc(N(c2ccccc2)c2cccc(N(c3ccccc3)c3ccc(-c4cccc5cc(-c6ccc7ccccc7c6)ccc45)cc3)c2)cc1 ; c1ccc(N(c2ccc(-c3ccc4cc(-c5ccc6ccccc6c5)ccc4c3)cc2)c2cccc3ccc4ccccc4c23)cc1]

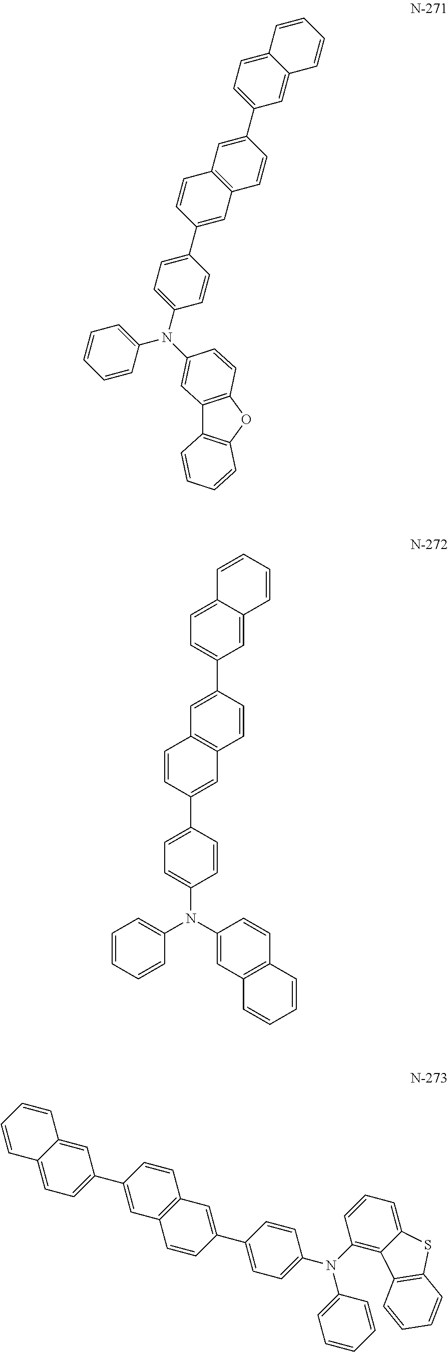 [SMILES: c1ccc(N(c2ccc(-c3ccc4cc(-c5ccc6ccccc6c5)ccc4c3)cc2)c2ccc3oc4ccccc4c3c2)cc1 ; c1ccc(N(c2ccc(-c3ccc4cc(-c5ccc6ccccc6c5)ccc4c3)cc2)c2ccc3ccccc3c2)cc1 ; c1ccc(N(c2ccc(-c3ccc4cc(-c5ccc6ccccc6c5)ccc4c3)cc2)c2cccc3sc4ccccc4c23)cc1]

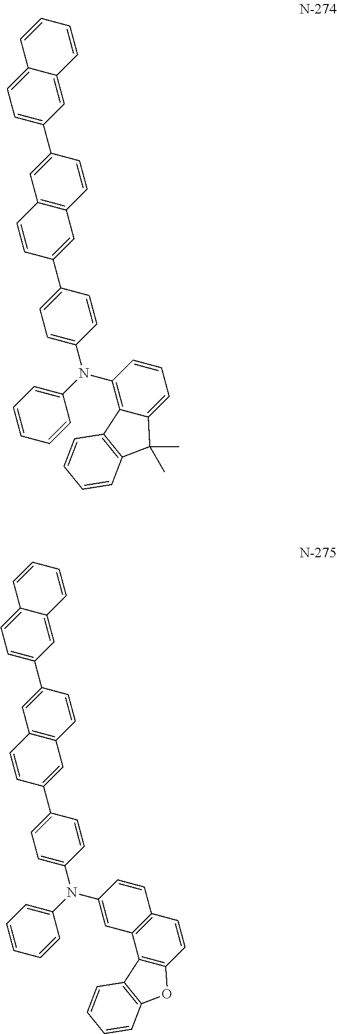 [SMILES: CC1(C)c2ccccc2-c2c(N(c3ccccc3)c3ccc(-c4ccc5cc(-c6ccc7ccccc7c6)ccc5c4)cc3)cccc21 ; c1ccc(N(c2ccc(-c3ccc4cc(-c5ccc6ccccc6c5)ccc4c3)cc2)c2ccc3ccc4oc5ccccc5c4c3c2)cc1]

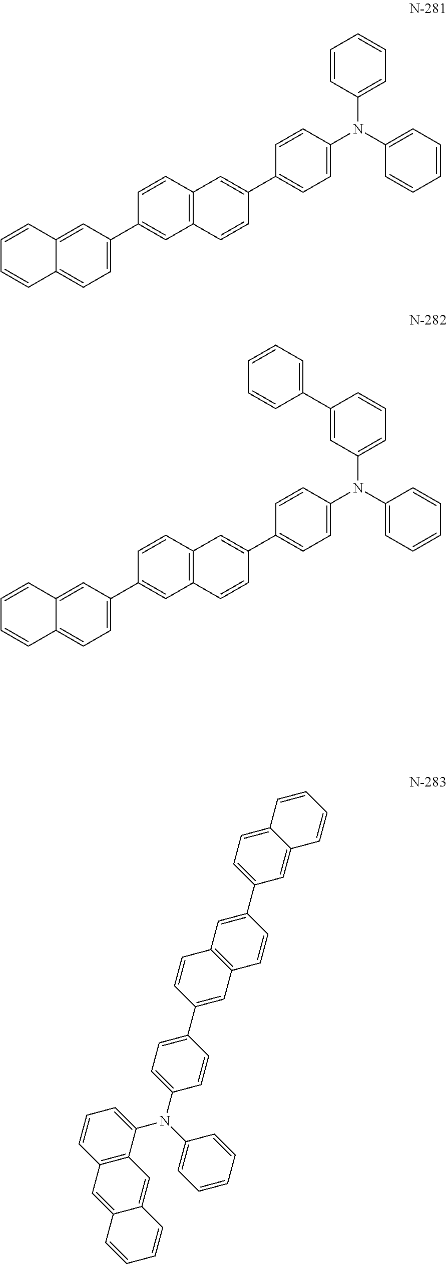 [SMILES: c1ccc(N(c2ccccc2)c2ccc(-c3ccc4cc(-c5ccc6ccccc6c5)ccc4c3)cc2)cc1 ; c1ccc(-c2cccc(N(c3ccccc3)c3ccc(-c4ccc5cc(-c6ccc7ccccc7c6)ccc5c4)cc3)c2)cc1 ; c1ccc(N(c2ccc(-c3ccc4cc(-c5ccc6ccccc6c5)ccc4c3)cc2)c2cccc3cc4ccccc4cc23)cc1]

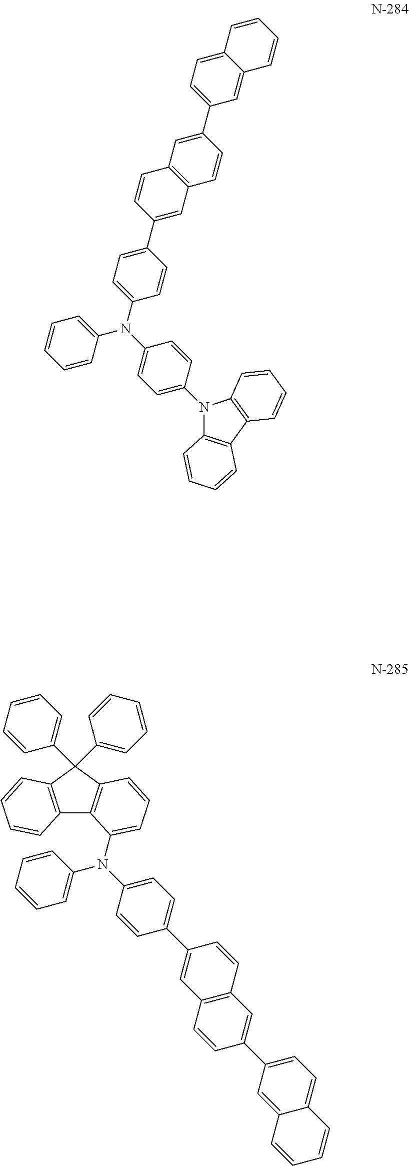 [SMILES: c1ccc(N(c2ccc(-c3ccc4cc(-c5ccc6ccccc6c5)ccc4c3)cc2)c2ccc(-n3c4ccccc4c4ccccc43)cc2)cc1 ; c1ccc(N(c2ccc(-c3ccc4cc(-c5ccc6ccccc6c5)ccc4c3)cc2)c2cccc3c2-c2ccccc2C3(c2ccccc2)c2ccccc2)cc1]

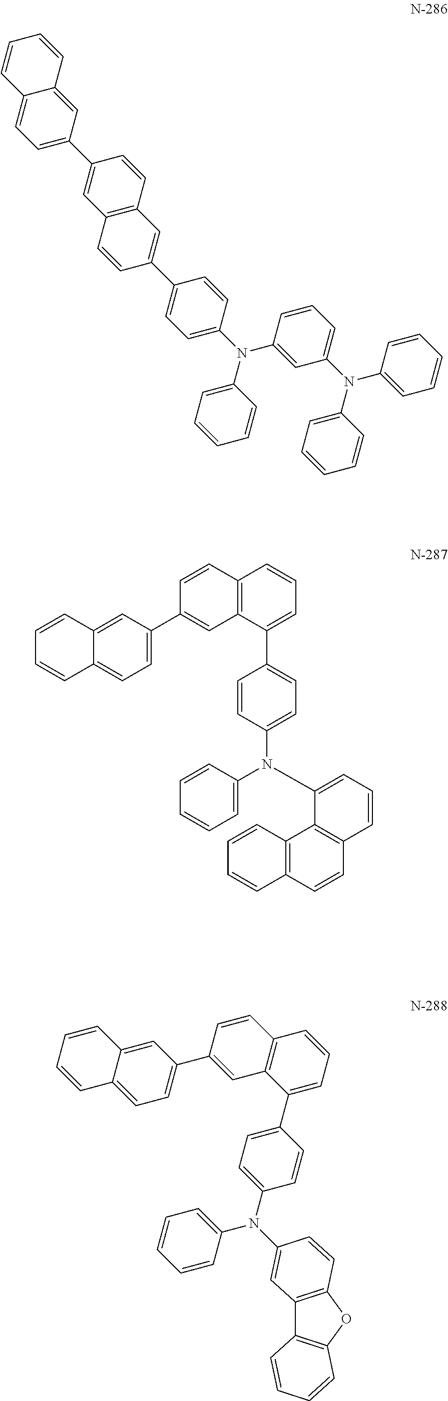 [SMILES: c1ccc(N(c2ccccc2)c2cccc(N(c3ccccc3)c3ccc(-c4ccc5cc(-c6ccc7ccccc7c6)ccc5c4)cc3)c2)cc1 ; c1ccc(N(c2ccc(-c3cccc4ccc(-c5ccc6ccccc6c5)cc34)cc2)c2cccc3ccc4ccccc4c23)cc1 ; c1ccc(N(c2ccc(-c3cccc4ccc(-c5ccc6ccccc6c5)cc34)cc2)c2ccc3oc4ccccc4c3c2)cc1]

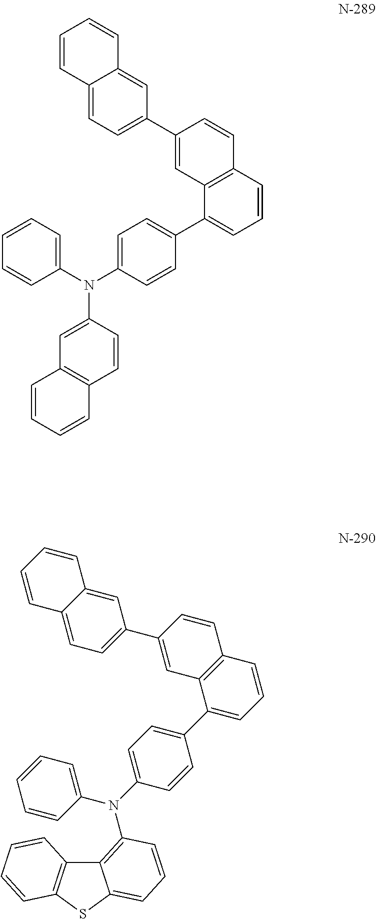 [SMILES: c1ccc(N(c2ccc(-c3cccc4ccc(-c5ccc6ccccc6c5)cc34)cc2)c2ccc3ccccc3c2)cc1 ; c1ccc(N(c2ccc(-c3cccc4ccc(-c5ccc6ccccc6c5)cc34)cc2)c2cccc3sc4ccccc4c23)cc1]

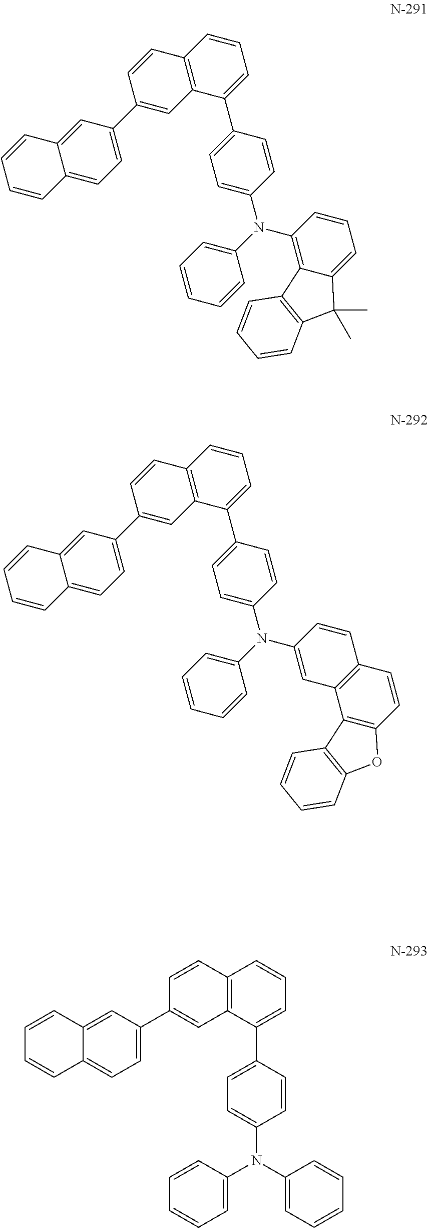 [SMILES: CC1(C)c2ccccc2-c2c(N(c3ccccc3)c3ccc(-c4cccc5ccc(-c6ccc7ccccc7c6)cc45)cc3)cccc21 ; c1ccc(N(c2ccc(-c3cccc4ccc(-c5ccc6ccccc6c5)cc34)cc2)c2ccc3ccc4oc5ccccc5c4c3c2)cc1 ; c1ccc(N(c2ccccc2)c2ccc(-c3cccc4ccc(-c5ccc6ccccc6c5)cc34)cc2)cc1]

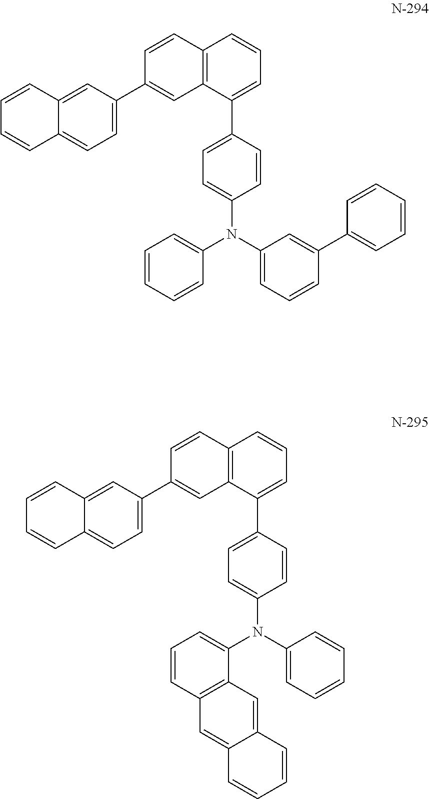 [SMILES: c1ccc(-c2cccc(N(c3ccccc3)c3ccc(-c4cccc5ccc(-c6ccc7ccccc7c6)cc45)cc3)c2)cc1 ; c1ccc(N(c2ccc(-c3cccc4ccc(-c5ccc6ccccc6c5)cc34)cc2)c2cccc3cc4ccccc4cc23)cc1]

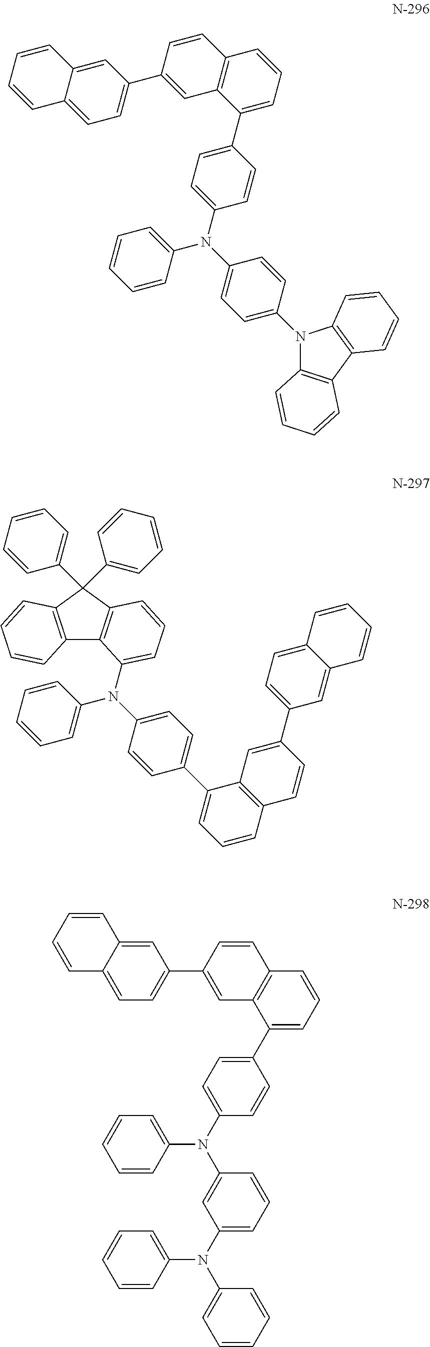 [SMILES: c1ccc(N(c2ccc(-c3cccc4ccc(-c5ccc6ccccc6c5)cc34)cc2)c2ccc(-n3c4ccccc4c4ccccc43)cc2)cc1 ; c1ccc(N(c2ccc(-c3cccc4ccc(-c5ccc6ccccc6c5)cc34)cc2)c2cccc3c2-c2ccccc2C3(c2ccccc2)c2ccccc2)cc1 ; c1ccc(N(c2ccccc2)c2cccc(N(c3ccccc3)c3ccc(-c4cccc5ccc(-c6ccc7ccccc7c6)cc45)cc3)c2)cc1]

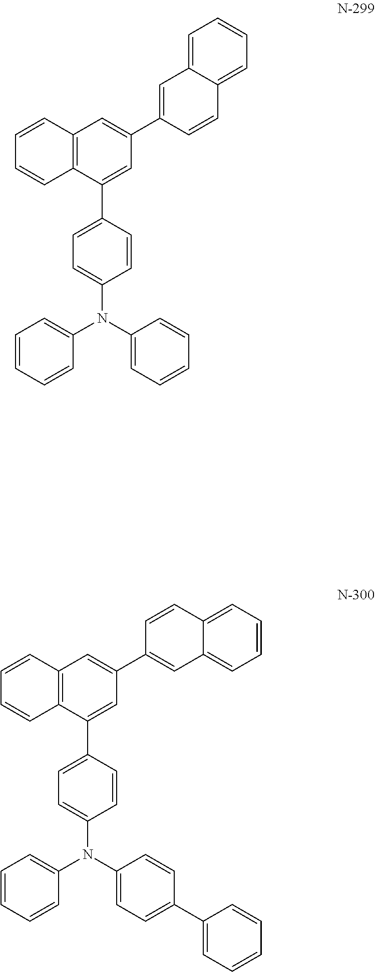 [SMILES: c1ccc(N(c2ccccc2)c2ccc(-c3cc(-c4ccc5ccccc5c4)cc4ccccc34)cc2)cc1 ; c1ccc(-c2ccc(N(c3ccccc3)c3ccc(-c4cc(-c5ccc6ccccc6c5)cc5ccccc45)cc3)cc2)cc1]

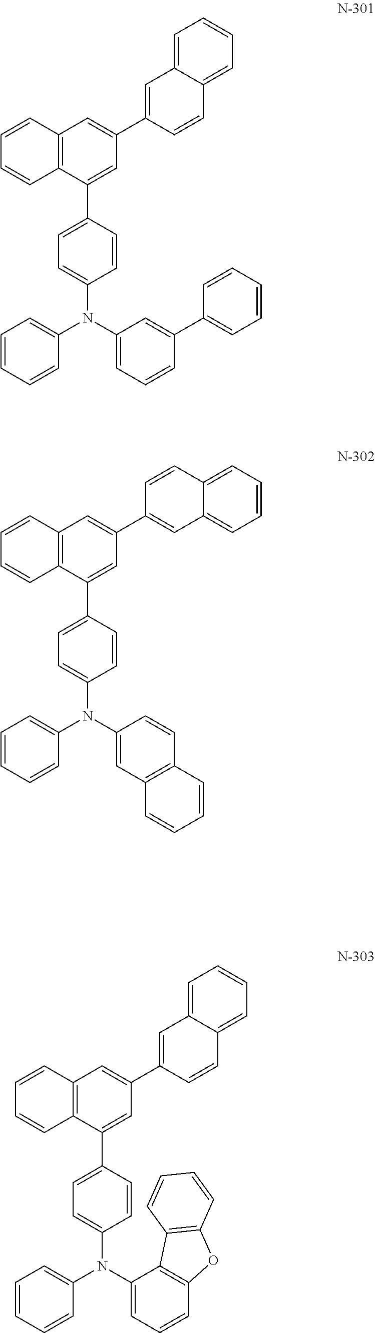 [SMILES: c1ccc(-c2cccc(N(c3ccccc3)c3ccc(-c4cc(-c5ccc6ccccc6c5)cc5ccccc45)cc3)c2)cc1 ; c1ccc(N(c2ccc(-c3cc(-c4ccc5ccccc5c4)cc4ccccc34)cc2)c2ccc3ccccc3c2)cc1 ; c1ccc(N(c2ccc(-c3cc(-c4ccc5ccccc5c4)cc4ccccc34)cc2)c2cccc3oc4ccccc4c23)cc1]

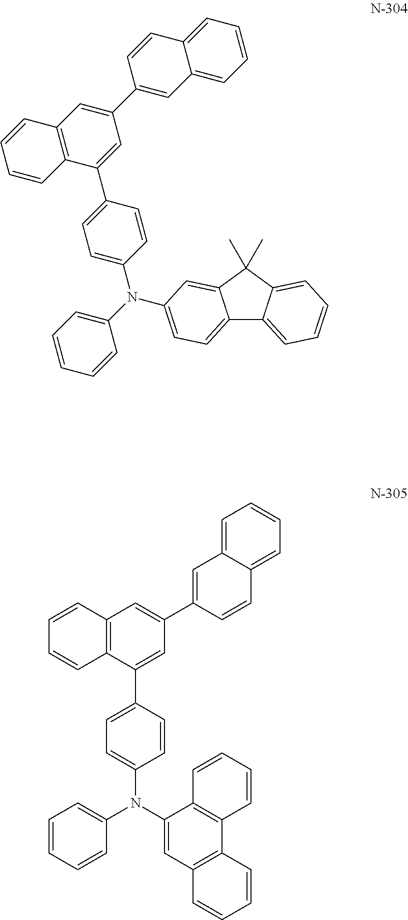 [SMILES: CC1(C)c2ccccc2-c2ccc(N(c3ccccc3)c3ccc(-c4cc(-c5ccc6ccccc6c5)cc5ccccc45)cc3)cc21 ; c1ccc(N(c2ccc(-c3cc(-c4ccc5ccccc5c4)cc4ccccc34)cc2)c2cc3ccccc3c3ccccc23)cc1]

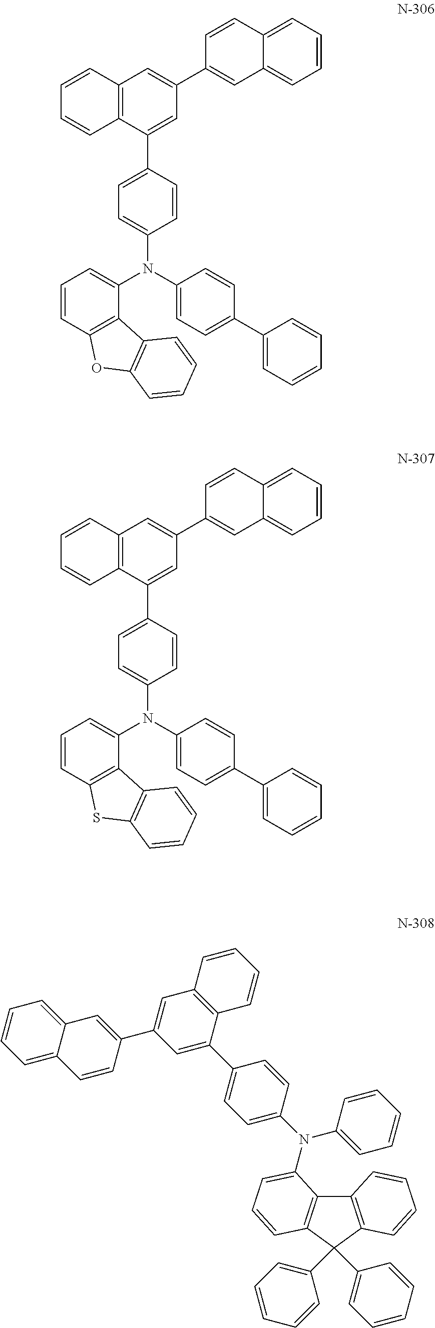 [SMILES: c1ccc(-c2ccc(N(c3ccc(-c4cc(-c5ccc6ccccc6c5)cc5ccccc45)cc3)c3cccc4oc5ccccc5c34)cc2)cc1 ; c1ccc(-c2ccc(N(c3ccc(-c4cc(-c5ccc6ccccc6c5)cc5ccccc45)cc3)c3cccc4sc5ccccc5c34)cc2)cc1 ; c1ccc(N(c2ccc(-c3cc(-c4ccc5ccccc5c4)cc4ccccc34)cc2)c2cccc3c2-c2ccccc2C3(c2ccccc2)c2ccccc2)cc1]

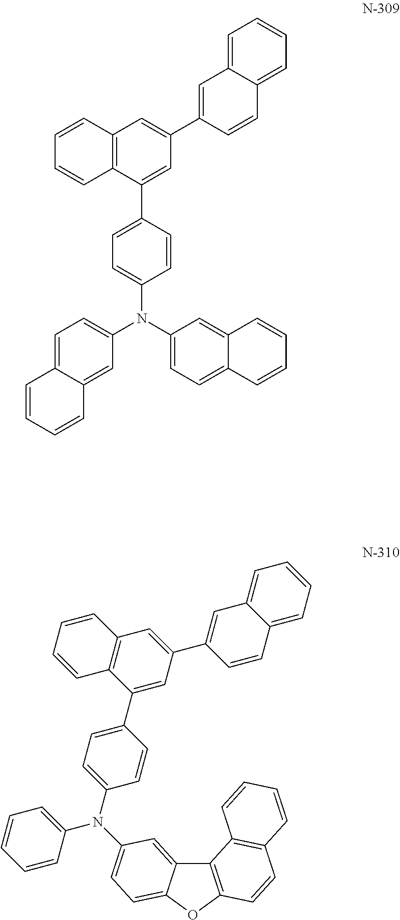 [SMILES: c1ccc2cc(-c3cc(-c4ccc(N(c5ccc6ccccc6c5)c5ccc6ccccc6c5)cc4)c4ccccc4c3)ccc2c1 ; c1ccc(N(c2ccc(-c3cc(-c4ccc5ccccc5c4)cc4ccccc34)cc2)c2ccc3oc4ccc5ccccc5c4c3c2)cc1]

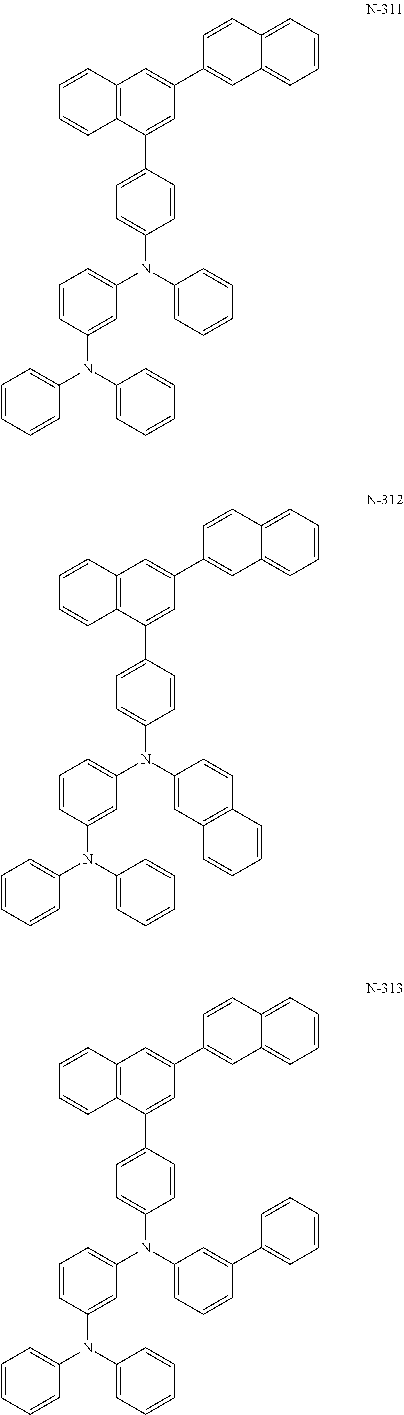 [SMILES: c1ccc(N(c2ccccc2)c2cccc(N(c3ccccc3)c3ccc(-c4cc(-c5ccc6ccccc6c5)cc5ccccc45)cc3)c2)cc1 ; c1ccc(N(c2ccccc2)c2cccc(N(c3ccc(-c4cc(-c5ccc6ccccc6c5)cc5ccccc45)cc3)c3ccc4ccccc4c3)c2)cc1 ; c1ccc(-c2cccc(N(c3ccc(-c4cc(-c5ccc6ccccc6c5)cc5ccccc45)cc3)c3cccc(N(c4ccccc4)c4ccccc4)c3)c2)cc1]

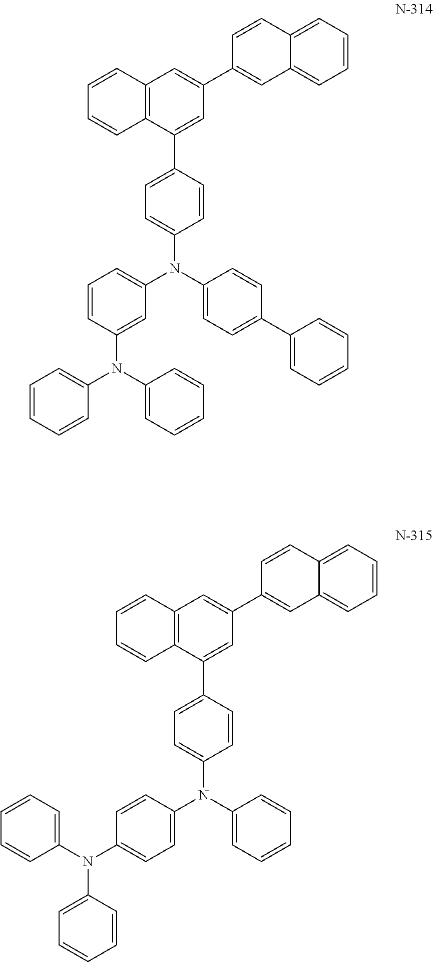 [SMILES: c1ccc(-c2ccc(N(c3ccc(-c4cc(-c5ccc6ccccc6c5)cc5ccccc45)cc3)c3cccc(N(c4ccccc4)c4ccccc4)c3)cc2)cc1 ; c1ccc(N(c2ccccc2)c2ccc(N(c3ccccc3)c3ccc(-c4cc(-c5ccc6ccccc6c5)cc5ccccc45)cc3)cc2)cc1]

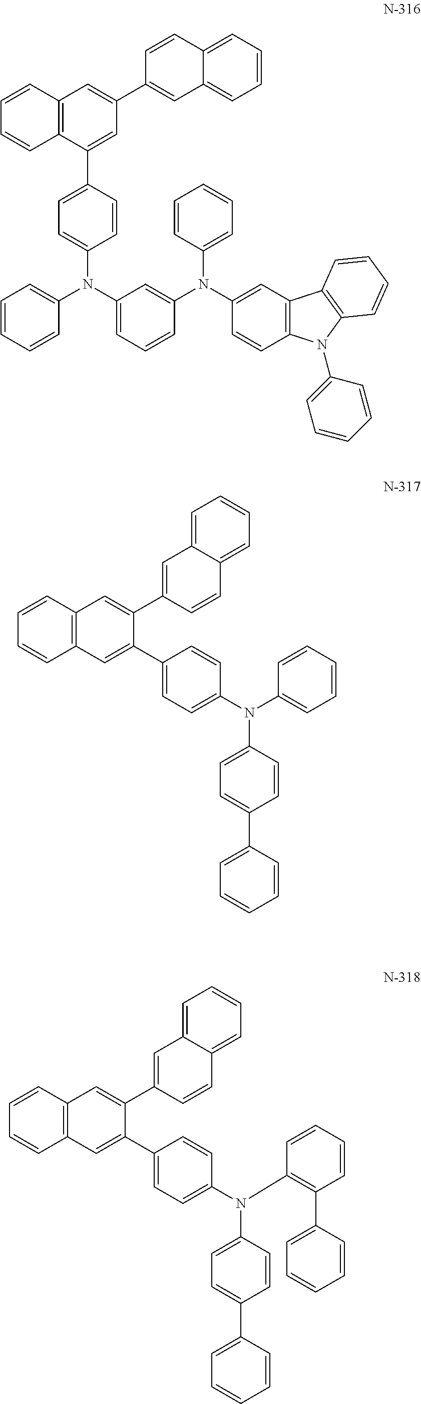 [SMILES: c1ccc(N(c2ccc(-c3cc(-c4ccc5ccccc5c4)cc4ccccc34)cc2)c2cccc(N(c3ccccc3)c3ccc4c(c3)c3ccccc3n4-c3ccccc3)c2)cc1 ; c1ccc(-c2ccc(N(c3ccccc3)c3ccc(-c4cc5ccccc5cc4-c4ccc5ccccc5c4)cc3)cc2)cc1 ; c1ccc(-c2ccc(N(c3ccc(-c4cc5ccccc5cc4-c4ccc5ccccc5c4)cc3)c3ccccc3-c3ccccc3)cc2)cc1]

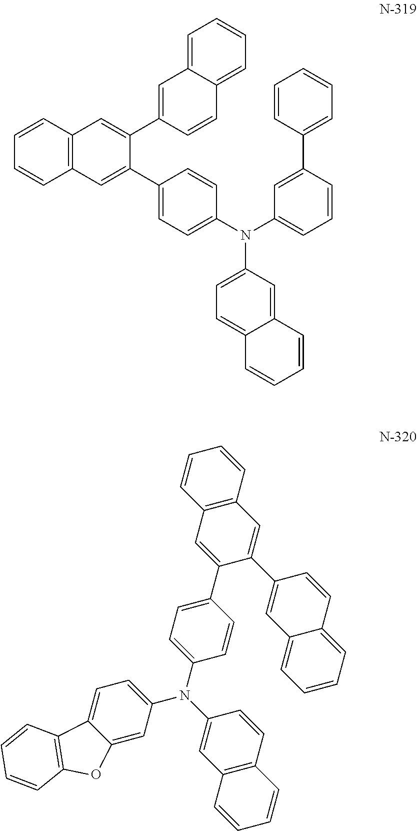 [SMILES: c1ccc(-c2cccc(N(c3ccc(-c4cc5ccccc5cc4-c4ccc5ccccc5c4)cc3)c3ccc4ccccc4c3)c2)cc1 ; c1ccc2cc(-c3cc4ccccc4cc3-c3ccc(N(c4ccc5ccccc5c4)c4ccc5c(c4)oc4ccccc45)cc3)ccc2c1]

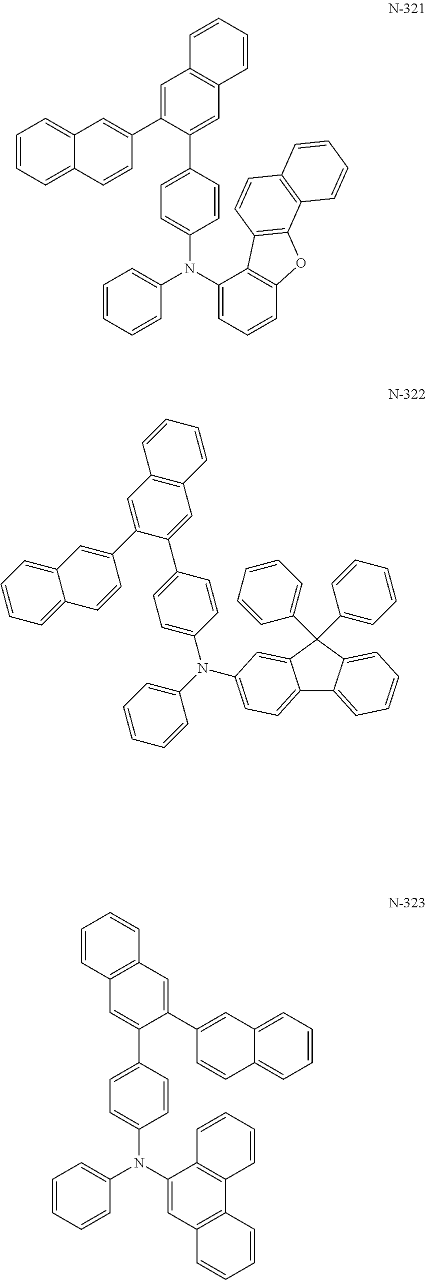 [SMILES: c1ccc(N(c2ccc(-c3cc4ccccc4cc3-c3ccc4ccccc4c3)cc2)c2cccc3oc4c5ccccc5ccc4c23)cc1 ; c1ccc(N(c2ccc(-c3cc4ccccc4cc3-c3ccc4ccccc4c3)cc2)c2ccc3c(c2)C(c2ccccc2)(c2ccccc2)c2ccccc2-3)cc1 ; c1ccc(N(c2ccc(-c3cc4ccccc4cc3-c3ccc4ccccc4c3)cc2)c2cc3ccccc3c3ccccc23)cc1]

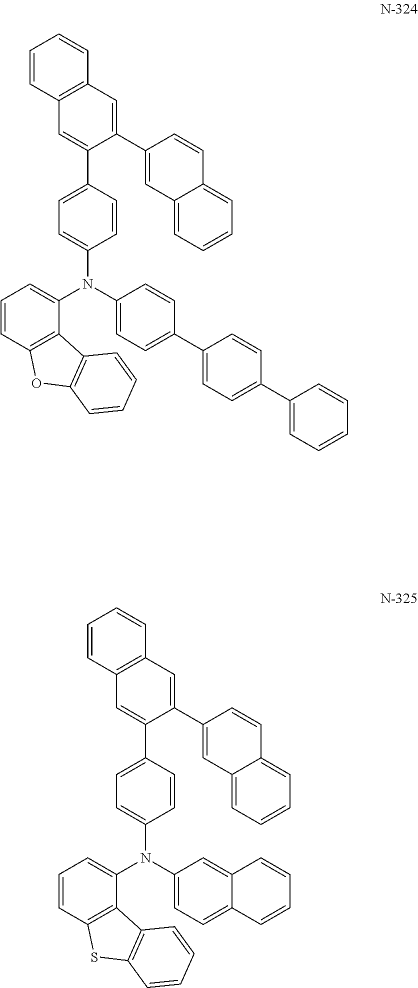 [SMILES: c1ccc(-c2ccc(-c3ccc(N(c4ccc(-c5cc6ccccc6cc5-c5ccc6ccccc6c5)cc4)c4cccc5oc6ccccc6c45)cc3)cc2)cc1 ; c1ccc2cc(-c3cc4ccccc4cc3-c3ccc(N(c4ccc5ccccc5c4)c4cccc5sc6ccccc6c45)cc3)ccc2c1]

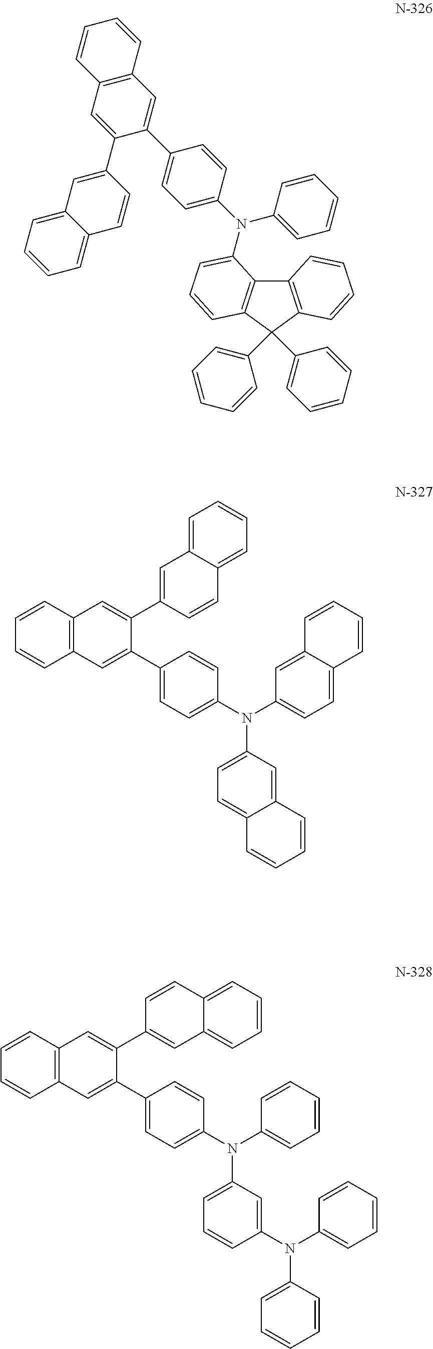 [SMILES: c1ccc(N(c2ccc(-c3cc4ccccc4cc3-c3ccc4ccccc4c3)cc2)c2cccc3c2-c2ccccc2C3(c2ccccc2)c2ccccc2)cc1 ; c1ccc2cc(-c3cc4ccccc4cc3-c3ccc(N(c4ccc5ccccc5c4)c4ccc5ccccc5c4)cc3)ccc2c1 ; c1ccc(N(c2ccccc2)c2cccc(N(c3ccccc3)c3ccc(-c4cc5ccccc5cc4-c4ccc5ccccc5c4)cc3)c2)cc1]

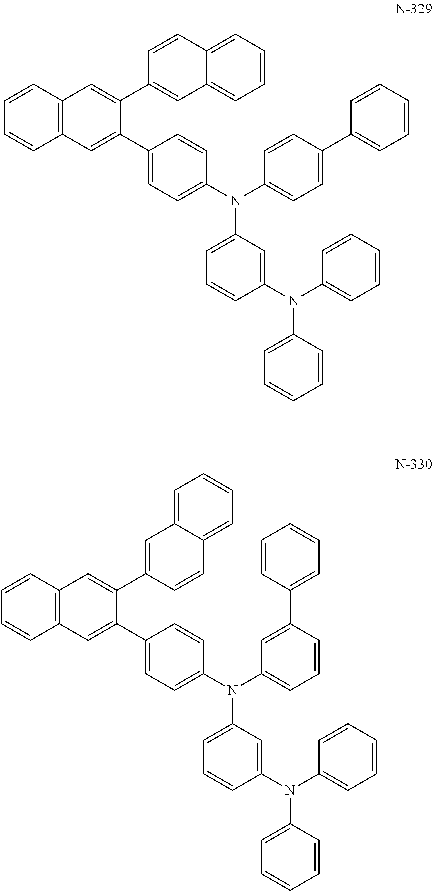 [SMILES: c1ccc(-c2ccc(N(c3ccc(-c4cc5ccccc5cc4-c4ccc5ccccc5c4)cc3)c3cccc(N(c4ccccc4)c4ccccc4)c3)cc2)cc1 ; c1ccc(-c2cccc(N(c3ccc(-c4cc5ccccc5cc4-c4ccc5ccccc5c4)cc3)c3cccc(N(c4ccccc4)c4ccccc4)c3)c2)cc1]

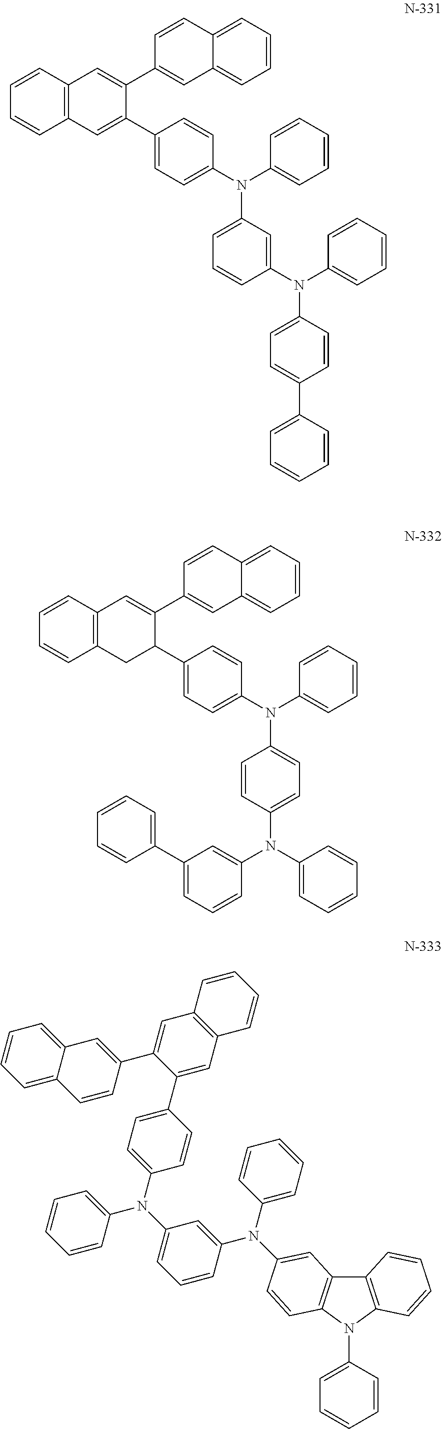 [SMILES: c1ccc(-c2ccc(N(c3ccccc3)c3cccc(N(c4ccccc4)c4ccc(-c5cc6ccccc6cc5-c5ccc6ccccc6c5)cc4)c3)cc2)cc1 ; C1=C(c2ccc3ccccc3c2)C(c2ccc(N(c3ccccc3)c3ccc(N(c4ccccc4)c4cccc(-c5ccccc5)c4)cc3)cc2)Cc2ccccc21 ; c1ccc(N(c2ccc(-c3cc4ccccc4cc3-c3ccc4ccccc4c3)cc2)c2cccc(N(c3ccccc3)c3ccc4c(c3)c3ccccc3n4-c3ccccc3)c2)cc1]

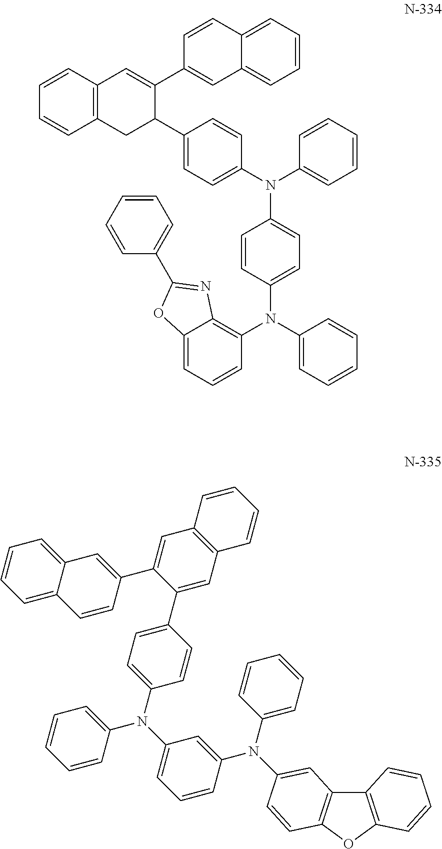 [SMILES: C1=C(c2ccc3ccccc3c2)C(c2ccc(N(c3ccccc3)c3ccc(N(c4ccccc4)c4cccc5oc(-c6ccccc6)nc45)cc3)cc2)Cc2ccccc21 ; c1ccc(N(c2ccc(-c3cc4ccccc4cc3-c3ccc4ccccc4c3)cc2)c2cccc(N(c3ccccc3)c3ccc4oc5ccccc5c4c3)c2)cc1]

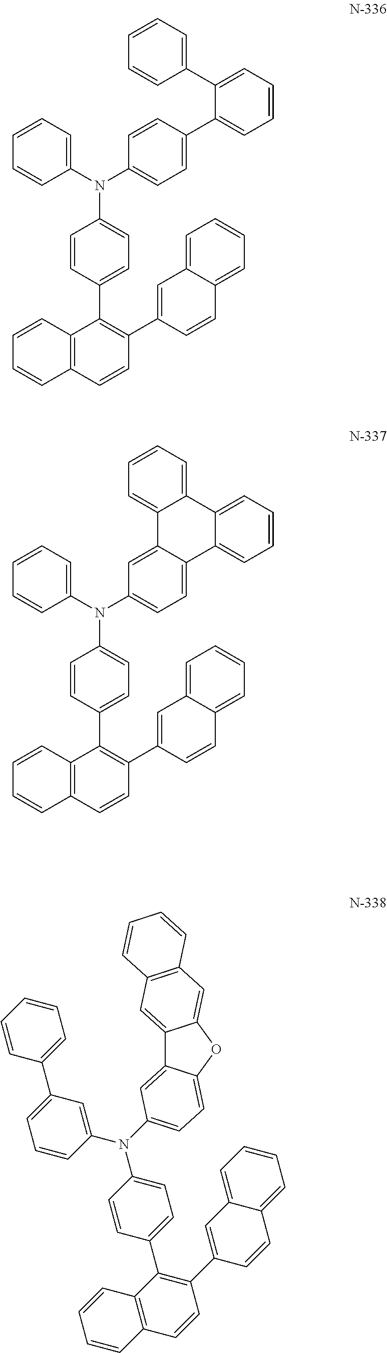 [SMILES: c1ccc(-c2ccccc2-c2ccc(N(c3ccccc3)c3ccc(-c4c(-c5ccc6ccccc6c5)ccc5ccccc45)cc3)cc2)cc1 ; c1ccc(N(c2ccc(-c3c(-c4ccc5ccccc5c4)ccc4ccccc34)cc2)c2ccc3c4ccccc4c4ccccc4c3c2)cc1 ; c1ccc(-c2cccc(N(c3ccc(-c4c(-c5ccc6ccccc6c5)ccc5ccccc45)cc3)c3ccc4oc5cc6ccccc6cc5c4c3)c2)cc1]

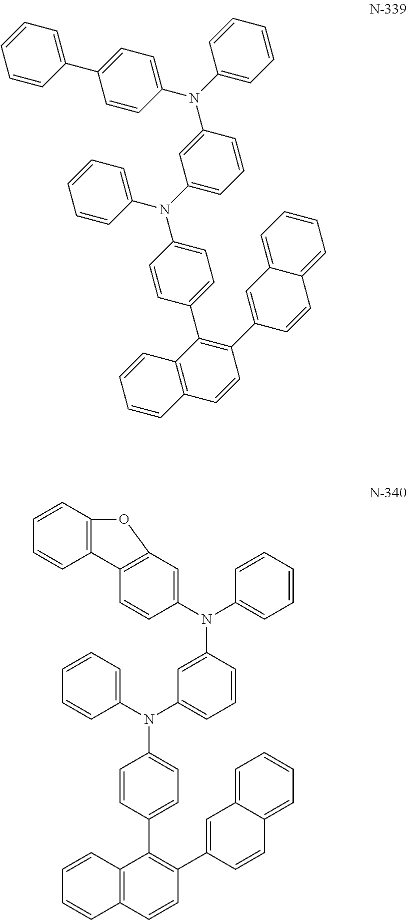 [SMILES: c1ccc(-c2ccc(N(c3ccccc3)c3cccc(N(c4ccccc4)c4ccc(-c5c(-c6ccc7ccccc7c6)ccc6ccccc56)cc4)c3)cc2)cc1 ; c1ccc(N(c2ccc(-c3c(-c4ccc5ccccc5c4)ccc4ccccc34)cc2)c2cccc(N(c3ccccc3)c3ccc4c(c3)oc3ccccc34)c2)cc1]

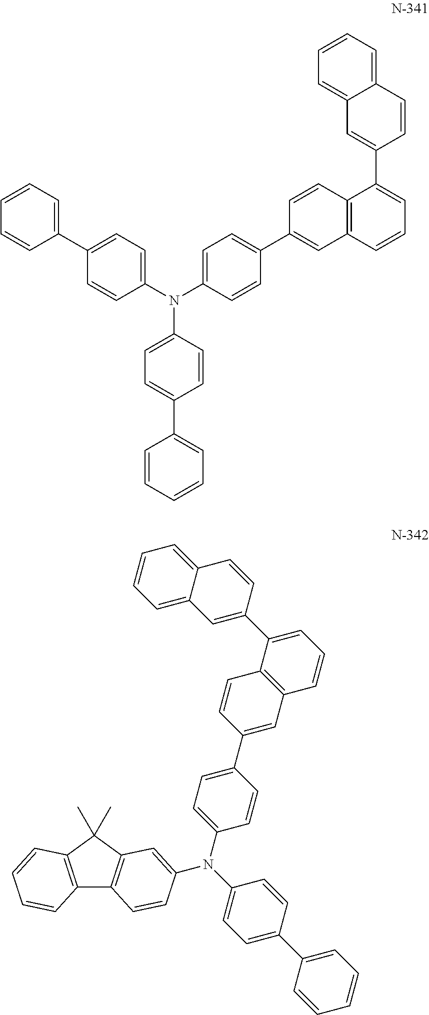

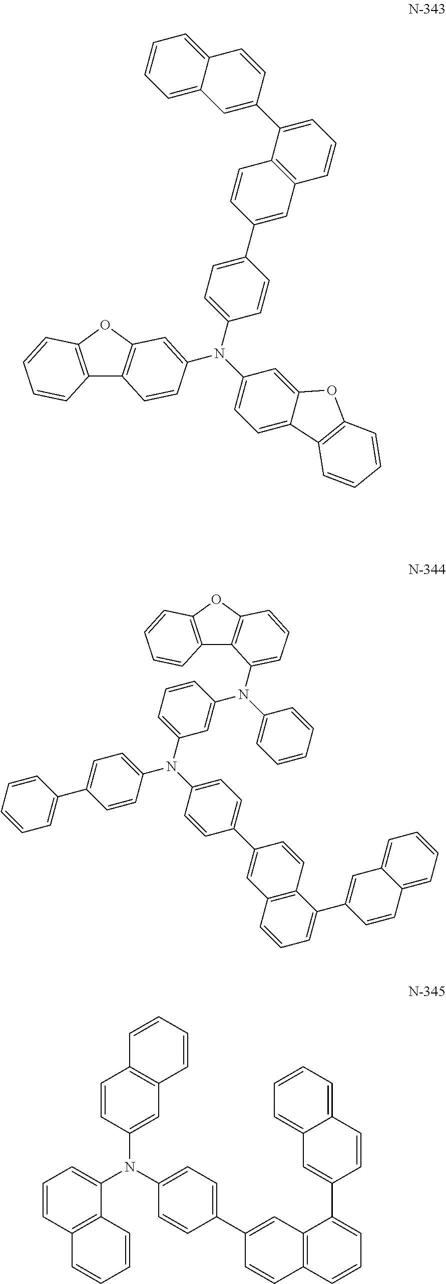 [SMILES: c1ccc2cc(-c3cccc4cc(-c5ccc(N(c6ccc7c(c6)oc6ccccc67)c6ccc7c(c6)oc6ccccc67)cc5)ccc34)ccc2c1 ; c1ccc(-c2ccc(N(c3ccc(-c4ccc5c(-c6ccc7ccccc7c6)cccc5c4)cc3)c3cccc(N(c4ccccc4)c4cccc5oc6ccccc6c45)c3)cc2)cc1 ; c1ccc2cc(-c3cccc4ccc(-c5ccc(N(c6ccc7ccccc7c6)c6cccc7ccccc67)cc5)cc34)ccc2c1]

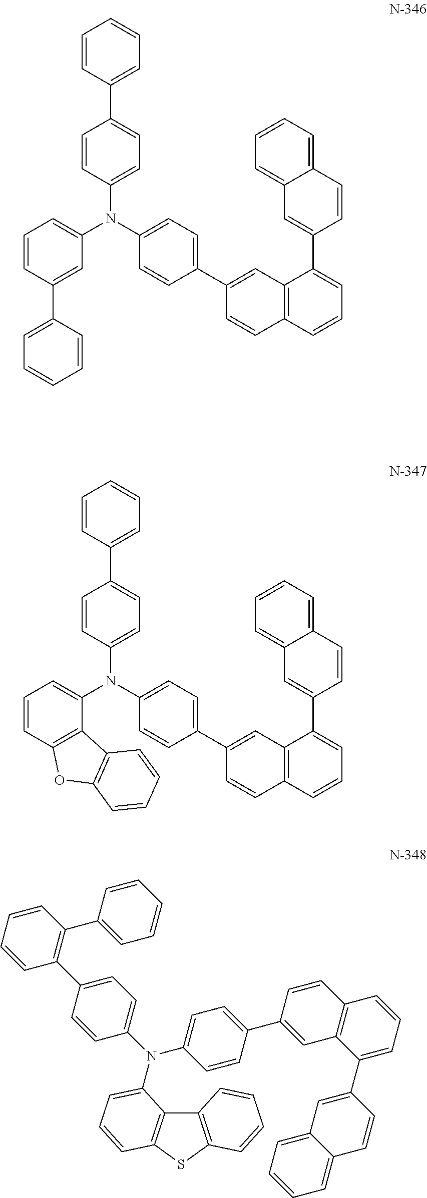 [SMILES: c1ccc(-c2ccc(N(c3ccc(-c4ccc5cccc(-c6ccc7ccccc7c6)c5c4)cc3)c3cccc(-c4ccccc4)c3)cc2)cc1 ; c1ccc(-c2ccc(N(c3ccc(-c4ccc5cccc(-c6ccc7ccccc7c6)c5c4)cc3)c3cccc4oc5ccccc5c34)cc2)cc1 ; c1ccc(-c2ccccc2-c2ccc(N(c3ccc(-c4ccc5cccc(-c6ccc7ccccc7c6)c5c4)cc3)c3cccc4sc5ccccc5c34)cc2)cc1]

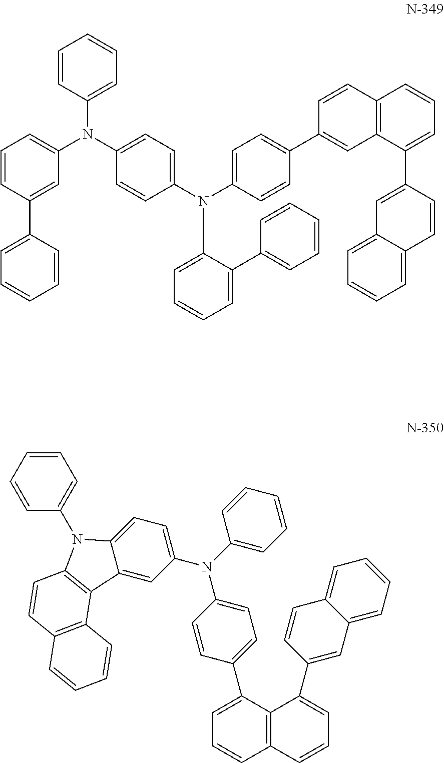 [SMILES: c1ccc(-c2cccc(N(c3ccccc3)c3ccc(N(c4ccc(-c5ccc6cccc(-c7ccc8ccccc8c7)c6c5)cc4)c4ccccc4-c4ccccc4)cc3)c2)cc1 ; c1ccc(N(c2ccc(-c3cccc4cccc(-c5ccc6ccccc6c5)c34)cc2)c2ccc3c(c2)c2c4ccccc4ccc2n3-c2ccccc2)cc1]

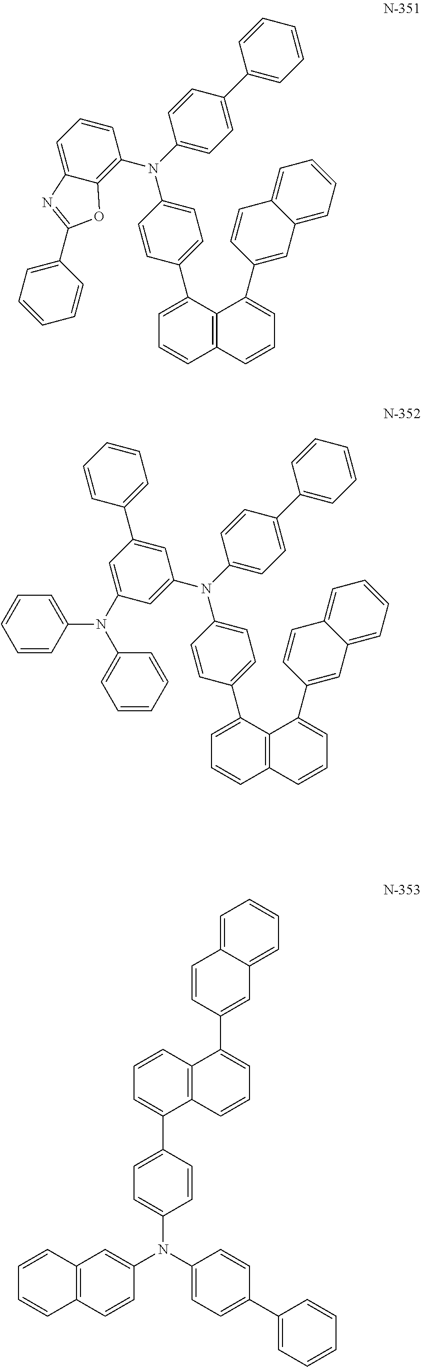 [SMILES: c1ccc(-c2ccc(N(c3ccc(-c4cccc5cccc(-c6ccc7ccccc7c6)c45)cc3)c3cccc4nc(-c5ccccc5)oc34)cc2)cc1 ; c1ccc(-c2ccc(N(c3ccc(-c4cccc5cccc(-c6ccc7ccccc7c6)c45)cc3)c3cc(-c4ccccc4)cc(N(c4ccccc4)c4ccccc4)c3)cc2)cc1 ; c1ccc(-c2ccc(N(c3ccc(-c4cccc5c(-c6ccc7ccccc7c6)cccc45)cc3)c3ccc4ccccc4c3)cc2)cc1]

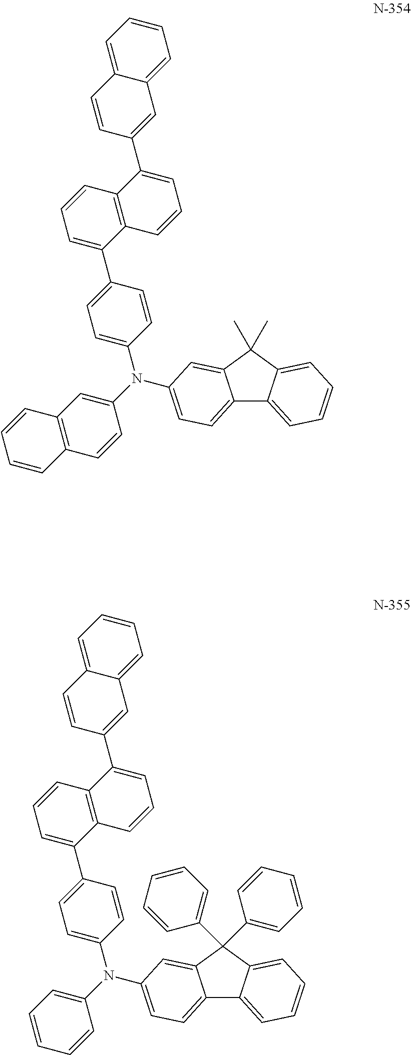 [SMILES: CC1(C)c2ccccc2-c2ccc(N(c3ccc(-c4cccc5c(-c6ccc7ccccc7c6)cccc45)cc3)c3ccc4ccccc4c3)cc21 ; c1ccc(N(c2ccc(-c3cccc4c(-c5ccc6ccccc6c5)cccc34)cc2)c2ccc3c(c2)C(c2ccccc2)(c2ccccc2)c2ccccc2-3)cc1]

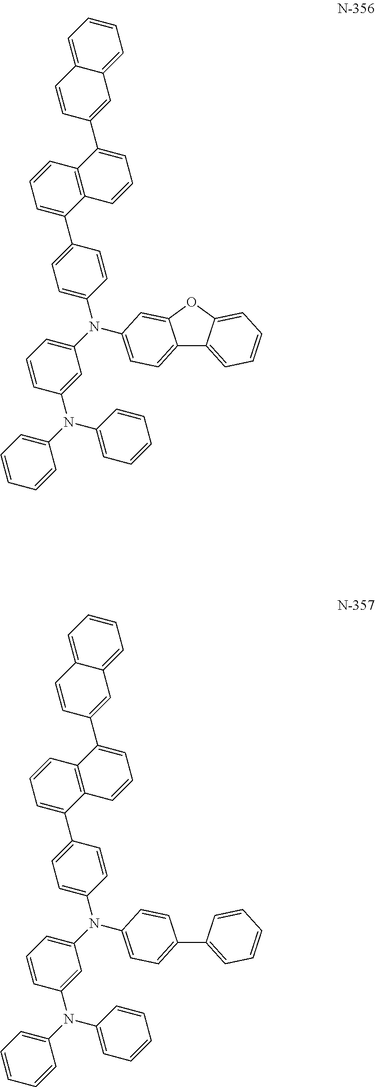 [SMILES: c1ccc(N(c2ccccc2)c2cccc(N(c3ccc(-c4cccc5c(-c6ccc7ccccc7c6)cccc45)cc3)c3ccc4c(c3)oc3ccccc34)c2)cc1 ; c1ccc(-c2ccc(N(c3ccc(-c4cccc5c(-c6ccc7ccccc7c6)cccc45)cc3)c3cccc(N(c4ccccc4)c4ccccc4)c3)cc2)cc1]

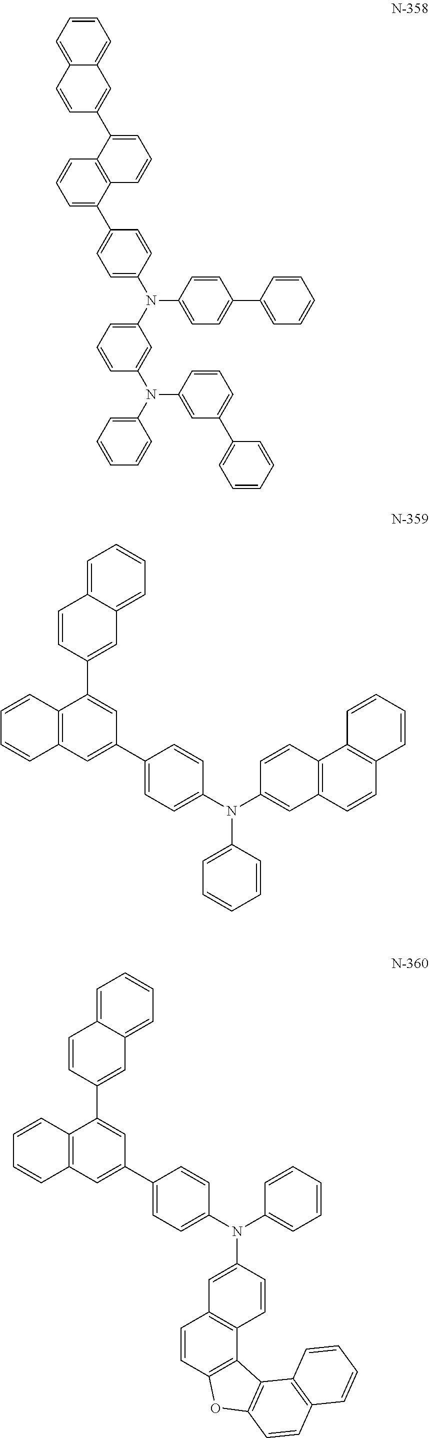 [SMILES: c1ccc(-c2ccc(N(c3ccc(-c4cccc5c(-c6ccc7ccccc7c6)cccc45)cc3)c3cccc(N(c4ccccc4)c4cccc(-c5ccccc5)c4)c3)cc2)cc1 ; c1ccc(N(c2ccc(-c3cc(-c4ccc5ccccc5c4)c4ccccc4c3)cc2)c2ccc3c(ccc4ccccc43)c2)cc1 ; c1ccc(N(c2ccc(-c3cc(-c4ccc5ccccc5c4)c4ccccc4c3)cc2)c2ccc3c(ccc4oc5ccc6ccccc6c5c43)c2)cc1]

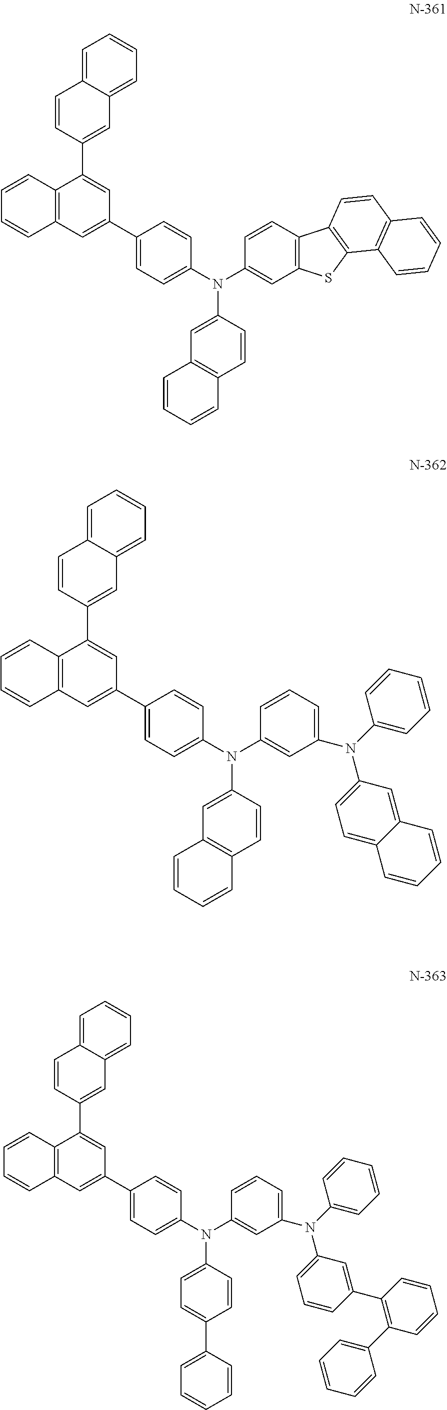 [SMILES: c1ccc2cc(-c3cc(-c4ccc(N(c5ccc6ccccc6c5)c5ccc6c(c5)sc5c7ccccc7ccc65)cc4)cc4ccccc34)ccc2c1 ; c1ccc(N(c2cccc(N(c3ccc(-c4cc(-c5ccc6ccccc6c5)c5ccccc5c4)cc3)c3ccc4ccccc4c3)c2)c2ccc3ccccc3c2)cc1 ; c1ccc(-c2ccc(N(c3ccc(-c4cc(-c5ccc6ccccc6c5)c5ccccc5c4)cc3)c3cccc(N(c4ccccc4)c4cccc(-c5ccccc5-c5ccccc5)c4)c3)cc2)cc1]

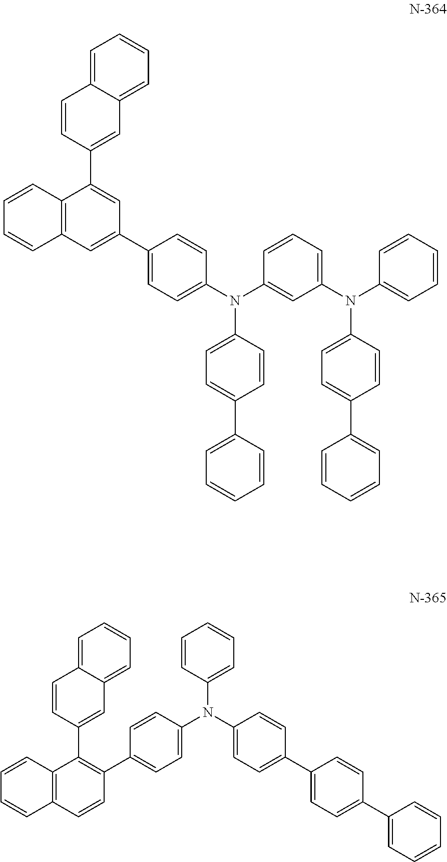 [SMILES: c1ccc(-c2ccc(N(c3ccccc3)c3cccc(N(c4ccc(-c5ccccc5)cc4)c4ccc(-c5cc(-c6ccc7ccccc7c6)c6ccccc6c5)cc4)c3)cc2)cc1 ; c1ccc(-c2ccc(-c3ccc(N(c4ccccc4)c4ccc(-c5ccc6ccccc6c5-c5ccc6ccccc6c5)cc4)cc3)cc2)cc1]

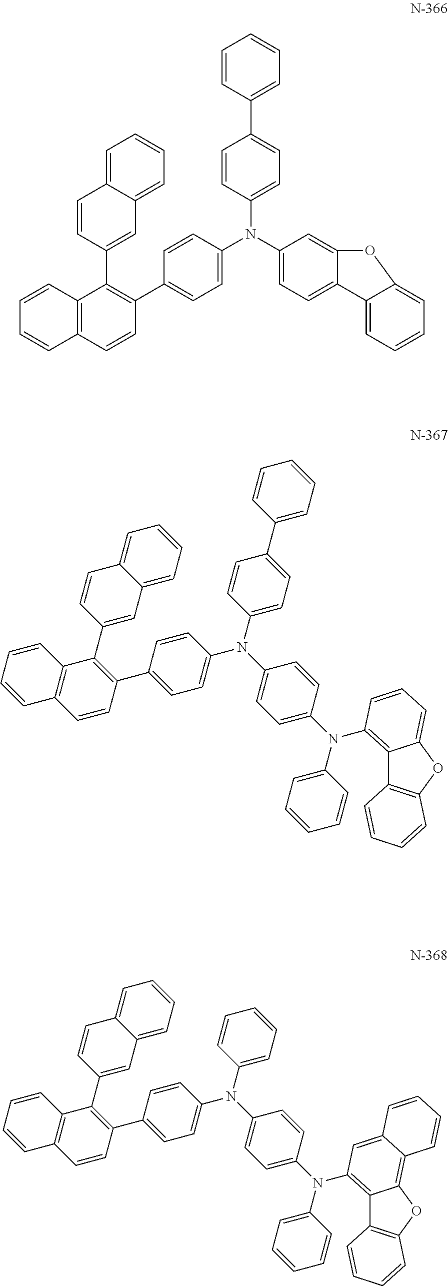 [SMILES: c1ccc(-c2ccc(N(c3ccc(-c4ccc5ccccc5c4-c4ccc5ccccc5c4)cc3)c3ccc4c(c3)oc3ccccc34)cc2)cc1 ; c1ccc(-c2ccc(N(c3ccc(-c4ccc5ccccc5c4-c4ccc5ccccc5c4)cc3)c3ccc(N(c4ccccc4)c4cccc5oc6ccccc6c45)cc3)cc2)cc1 ; c1ccc(N(c2ccc(-c3ccc4ccccc4c3-c3ccc4ccccc4c3)cc2)c2ccc(N(c3ccccc3)c3cc4ccccc4c4oc5ccccc5c34)cc2)cc1]

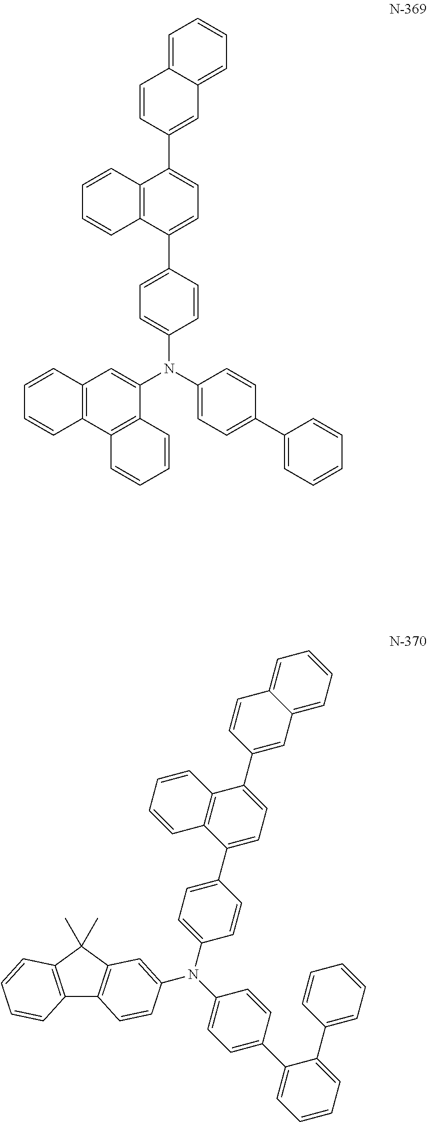 [SMILES: c1ccc(-c2ccc(N(c3ccc(-c4ccc(-c5ccc6ccccc6c5)c5ccccc45)cc3)c3cc4ccccc4c4ccccc34)cc2)cc1 ; CC1(C)c2ccccc2-c2ccc(N(c3ccc(-c4ccccc4-c4ccccc4)cc3)c3ccc(-c4ccc(-c5ccc6ccccc6c5)c5ccccc45)cc3)cc21]

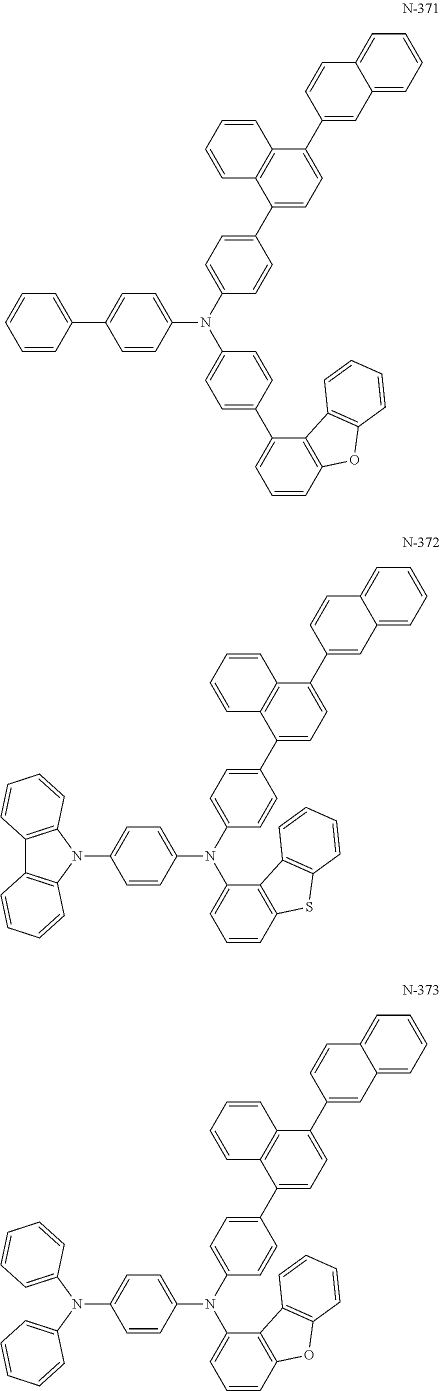 [SMILES: c1ccc(-c2ccc(N(c3ccc(-c4ccc(-c5ccc6ccccc6c5)c5ccccc45)cc3)c3ccc(-c4cccc5oc6ccccc6c45)cc3)cc2)cc1 ; c1ccc2cc(-c3ccc(-c4ccc(N(c5ccc(-n6c7ccccc7c7ccccc76)cc5)c5cccc6sc7ccccc7c56)cc4)c4ccccc34)ccc2c1 ; c1ccc(N(c2ccccc2)c2ccc(N(c3ccc(-c4ccc(-c5ccc6ccccc6c5)c5ccccc45)cc3)c3cccc4oc5ccccc5c34)cc2)cc1]

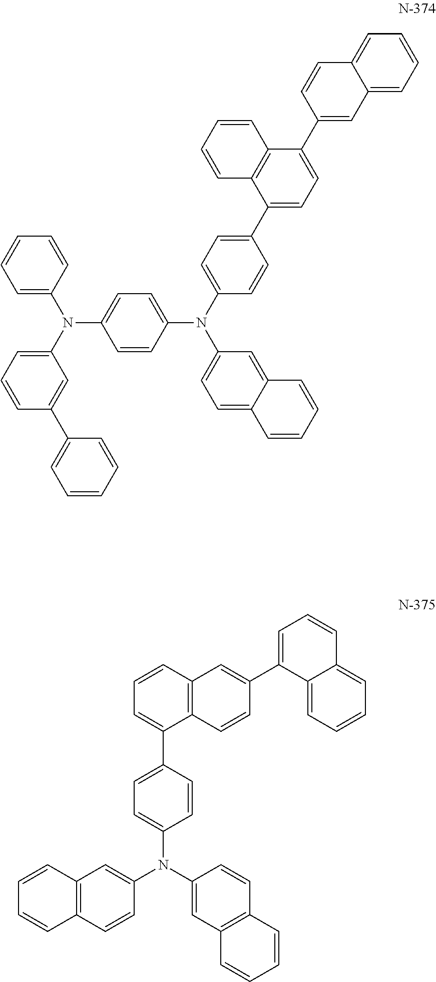 [SMILES: c1ccc(-c2cccc(N(c3ccccc3)c3ccc(N(c4ccc(-c5ccc(-c6ccc7ccccc7c6)c6ccccc56)cc4)c4ccc5ccccc5c4)cc3)c2)cc1 ; c1ccc2cc(N(c3ccc(-c4cccc5cc(-c6cccc7ccccc67)ccc45)cc3)c3ccc4ccccc4c3)ccc2c1]

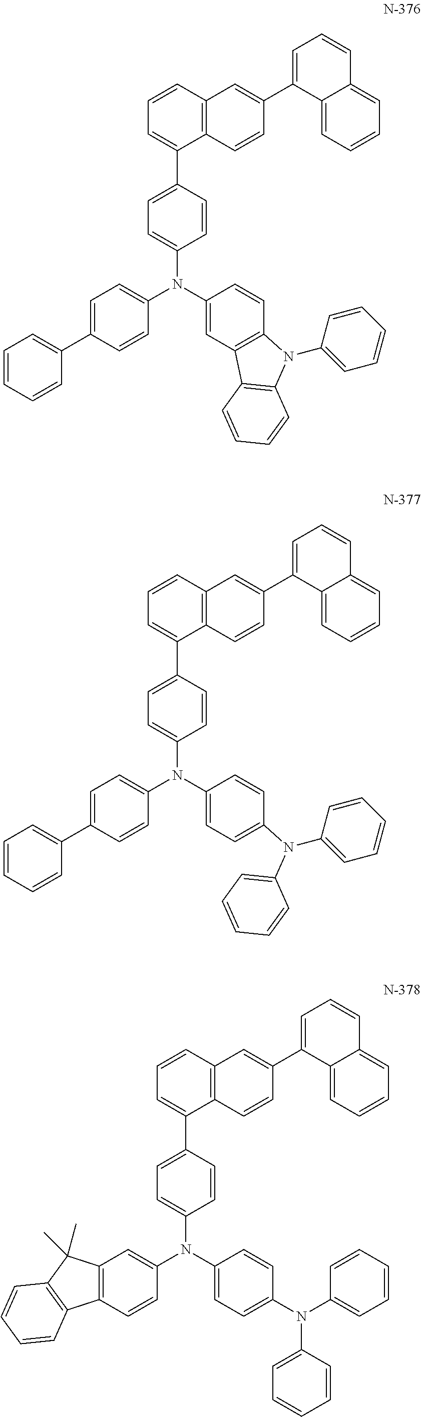 [SMILES: c1ccc(-c2ccc(N(c3ccc(-c4cccc5cc(-c6cccc7ccccc67)ccc45)cc3)c3ccc4c(c3)c3ccccc3n4-c3ccccc3)cc2)cc1 ; c1ccc(-c2ccc(N(c3ccc(-c4cccc5cc(-c6cccc7ccccc67)ccc45)cc3)c3ccc(N(c4ccccc4)c4ccccc4)cc3)cc2)cc1 ; CC1(C)c2ccccc2-c2ccc(N(c3ccc(-c4cccc5cc(-c6cccc7ccccc67)ccc45)cc3)c3ccc(N(c4ccccc4)c4ccccc4)cc3)cc21]

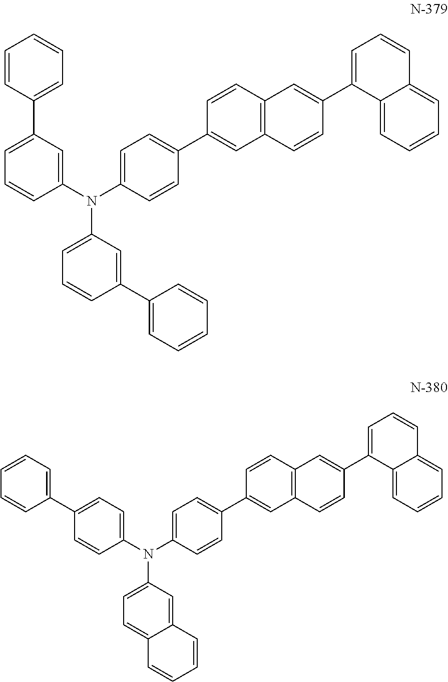 [SMILES: c1ccc(-c2cccc(N(c3ccc(-c4ccc5cc(-c6cccc7ccccc67)ccc5c4)cc3)c3cccc(-c4ccccc4)c3)c2)cc1 ; c1ccc(-c2ccc(N(c3ccc(-c4ccc5cc(-c6cccc7ccccc67)ccc5c4)cc3)c3ccc4ccccc4c3)cc2)cc1]

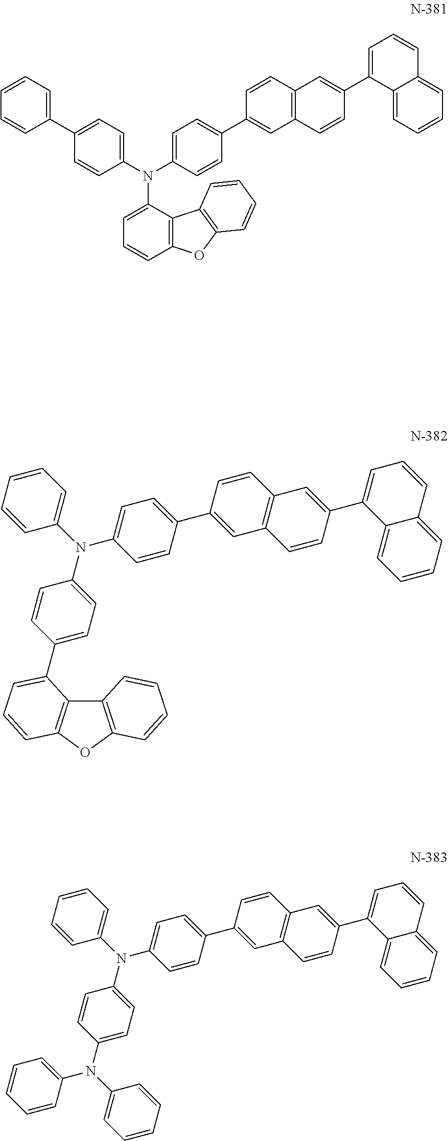 [SMILES: c1ccc(-c2ccc(N(c3ccc(-c4ccc5cc(-c6cccc7ccccc67)ccc5c4)cc3)c3cccc4oc5ccccc5c34)cc2)cc1 ; c1ccc(N(c2ccc(-c3ccc4cc(-c5cccc6ccccc56)ccc4c3)cc2)c2ccc(-c3cccc4oc5ccccc5c34)cc2)cc1 ; c1ccc(N(c2ccccc2)c2ccc(N(c3ccccc3)c3ccc(-c4ccc5cc(-c6cccc7ccccc67)ccc5c4)cc3)cc2)cc1]

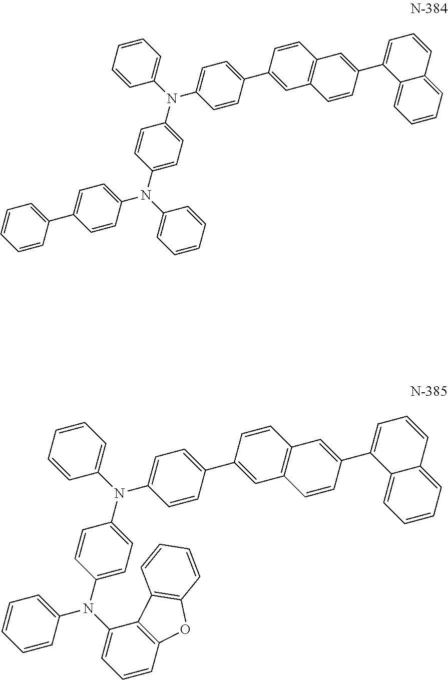 [SMILES: c1ccc(-c2ccc(N(c3ccccc3)c3ccc(N(c4ccccc4)c4ccc(-c5ccc6cc(-c7cccc8ccccc78)ccc6c5)cc4)cc3)cc2)cc1 ; c1ccc(N(c2ccc(-c3ccc4cc(-c5cccc6ccccc56)ccc4c3)cc2)c2ccc(N(c3ccccc3)c3cccc4oc5ccccc5c34)cc2)cc1]

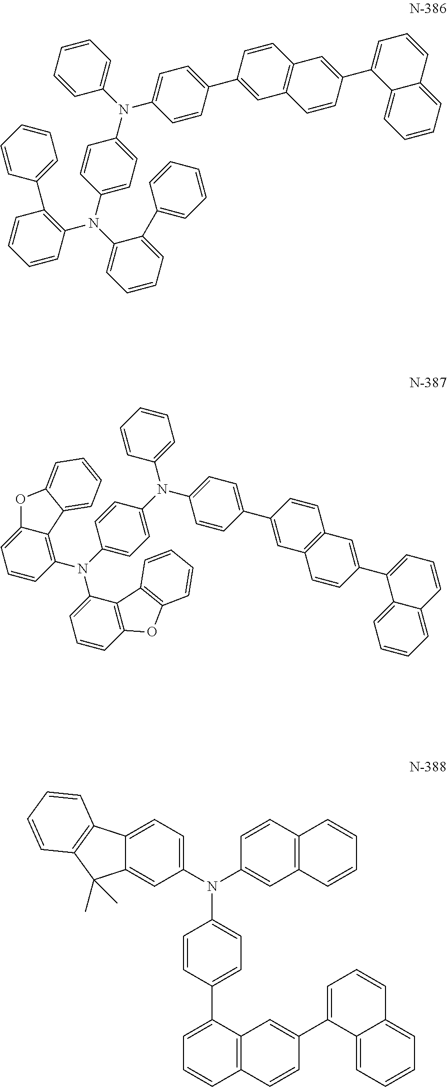 [SMILES: c1ccc(-c2ccccc2N(c2ccc(N(c3ccccc3)c3ccc(-c4ccc5cc(-c6cccc7ccccc67)ccc5c4)cc3)cc2)c2ccccc2-c2ccccc2)cc1 ; c1ccc(N(c2ccc(-c3ccc4cc(-c5cccc6ccccc56)ccc4c3)cc2)c2ccc(N(c3cccc4oc5ccccc5c34)c3cccc4oc5ccccc5c34)cc2)cc1 ; CC1(C)c2ccccc2-c2ccc(N(c3ccc(-c4cccc5ccc(-c6cccc7ccccc67)cc45)cc3)c3ccc4ccccc4c3)cc21]

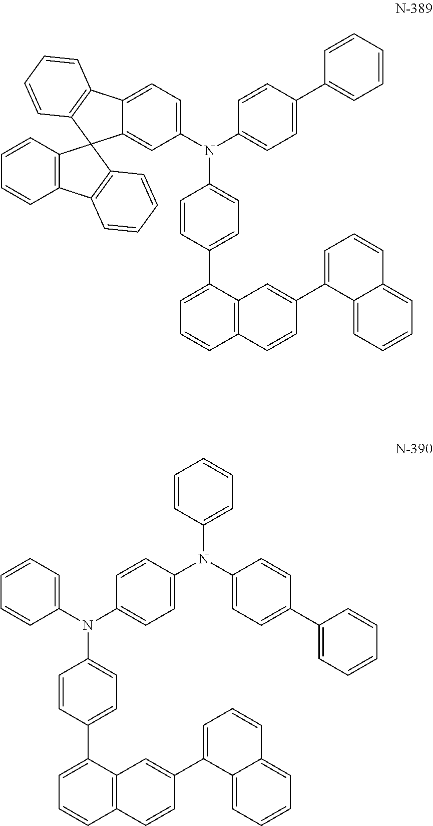 [SMILES: c1ccc(-c2ccc(N(c3ccc(-c4cccc5ccc(-c6cccc7ccccc67)cc45)cc3)c3ccc4c(c3)C3(c5ccccc5-c5ccccc53)c3ccccc3-4)cc2)cc1 ; c1ccc(-c2ccc(N(c3ccccc3)c3ccc(N(c4ccccc4)c4ccc(-c5cccc6ccc(-c7cccc8ccccc78)cc56)cc4)cc3)cc2)cc1]

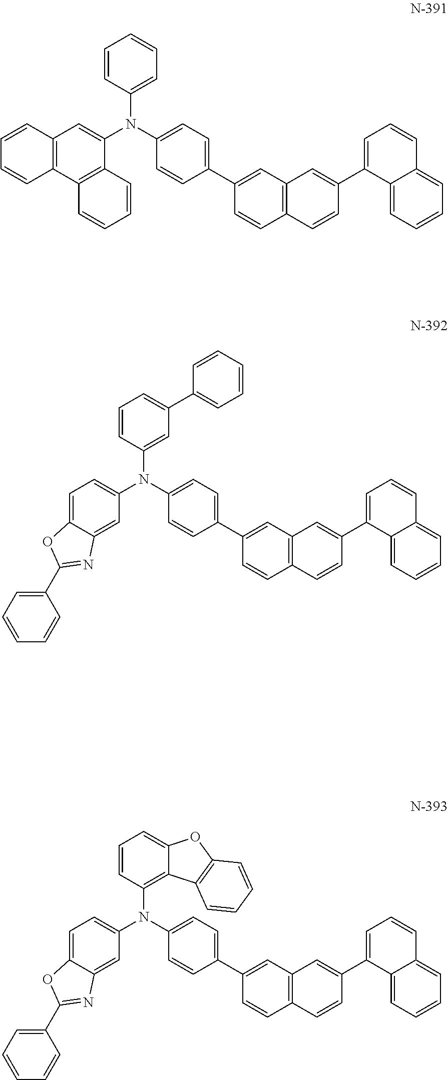 [SMILES: c1ccc(N(c2ccc(-c3ccc4ccc(-c5cccc6ccccc56)cc4c3)cc2)c2cc3ccccc3c3ccccc23)cc1 ; c1ccc(-c2cccc(N(c3ccc(-c4ccc5ccc(-c6cccc7ccccc67)cc5c4)cc3)c3ccc4oc(-c5ccccc5)nc4c3)c2)cc1 ; c1ccc(-c2nc3cc(N(c4ccc(-c5ccc6ccc(-c7cccc8ccccc78)cc6c5)cc4)c4cccc5oc6ccccc6c45)ccc3o2)cc1]

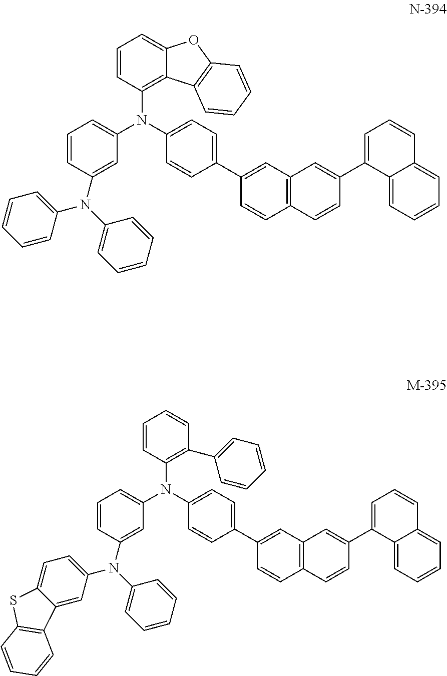 [SMILES: c1ccc(N(c2ccccc2)c2cccc(N(c3ccc(-c4ccc5ccc(-c6cccc7ccccc67)cc5c4)cc3)c3cccc4oc5ccccc5c34)c2)cc1 ; c1ccc(-c2ccccc2N(c2ccc(-c3ccc4ccc(-c5cccc6ccccc56)cc4c3)cc2)c2cccc(N(c3ccccc3)c3ccc4sc5ccccc5c4c3)c2)cc1]

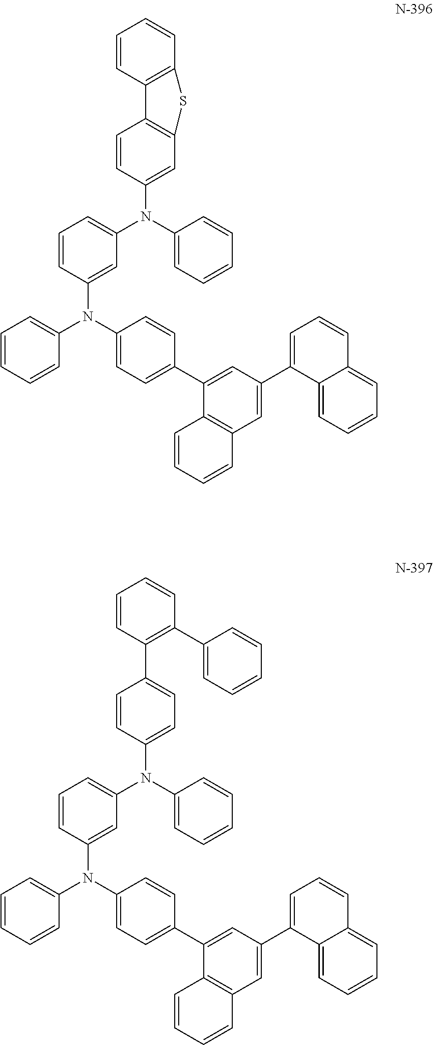 [SMILES: c1ccc(N(c2ccc(-c3cc(-c4cccc5ccccc45)cc4ccccc34)cc2)c2cccc(N(c3ccccc3)c3ccc4c(c3)sc3ccccc34)c2)cc1 ; c1ccc(-c2ccccc2-c2ccc(N(c3ccccc3)c3cccc(N(c4ccccc4)c4ccc(-c5cc(-c6cccc7ccccc67)cc6ccccc56)cc4)c3)cc2)cc1]

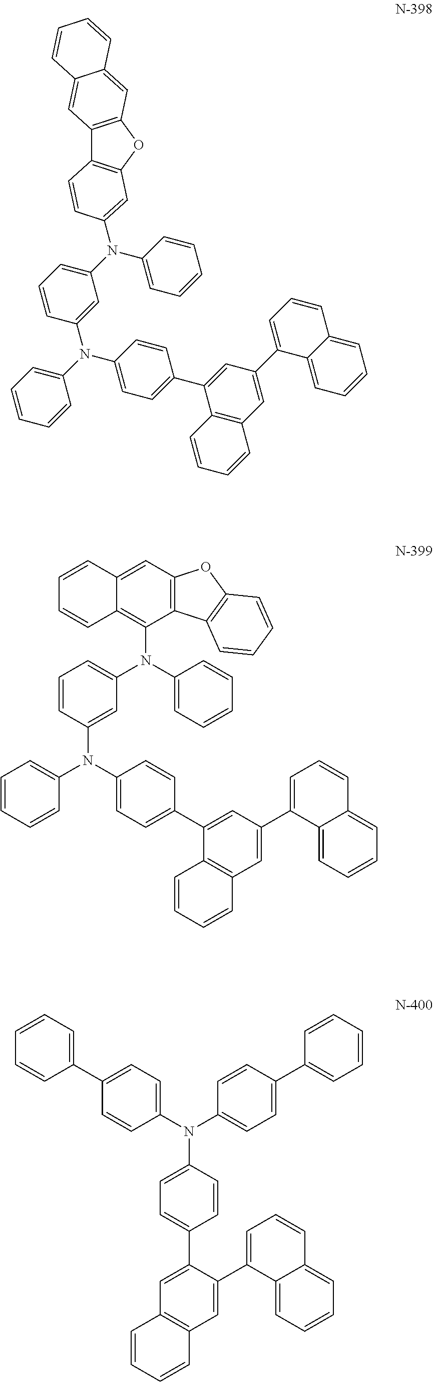 [SMILES: c1ccc(N(c2ccc(-c3cc(-c4cccc5ccccc45)cc4ccccc34)cc2)c2cccc(N(c3ccccc3)c3ccc4c(c3)oc3cc5ccccc5cc34)c2)cc1 ; c1ccc(N(c2ccc(-c3cc(-c4cccc5ccccc45)cc4ccccc34)cc2)c2cccc(N(c3ccccc3)c3c4ccccc4cc4oc5ccccc5c34)c2)cc1 ; c1ccc(-c2ccc(N(c3ccc(-c4ccccc4)cc3)c3ccc(-c4cc5ccccc5cc4-c4cccc5ccccc45)cc3)cc2)cc1]

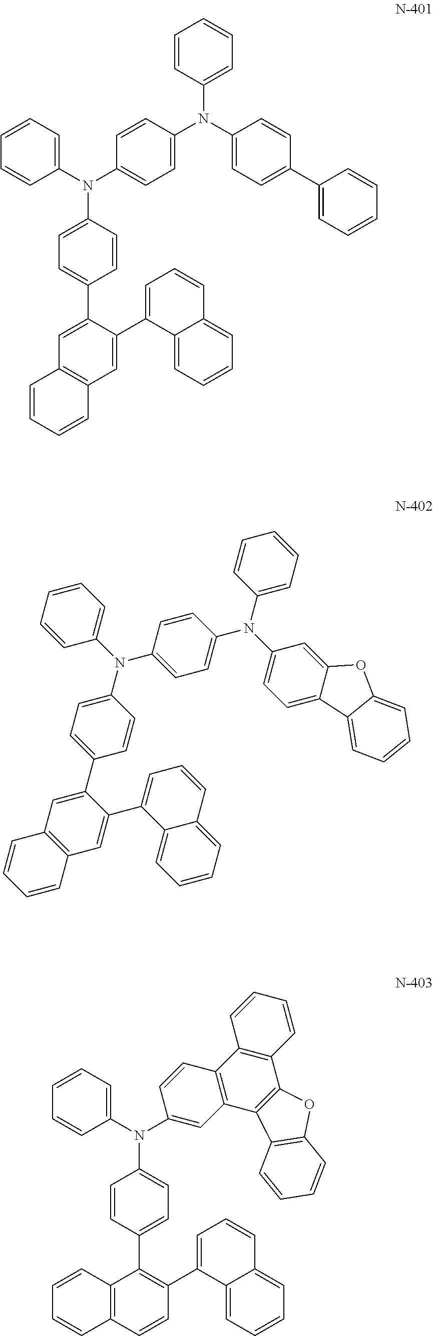 [SMILES: c1ccc(-c2ccc(N(c3ccccc3)c3ccc(N(c4ccccc4)c4ccc(-c5cc6ccccc6cc5-c5cccc6ccccc56)cc4)cc3)cc2)cc1 ; c1ccc(N(c2ccc(-c3cc4ccccc4cc3-c3cccc4ccccc34)cc2)c2ccc(N(c3ccccc3)c3ccc4c(c3)oc3ccccc34)cc2)cc1 ; c1ccc(N(c2ccc(-c3c(-c4cccc5ccccc45)ccc4ccccc34)cc2)c2ccc3c4ccccc4c4oc5ccccc5c4c3c2)cc1]

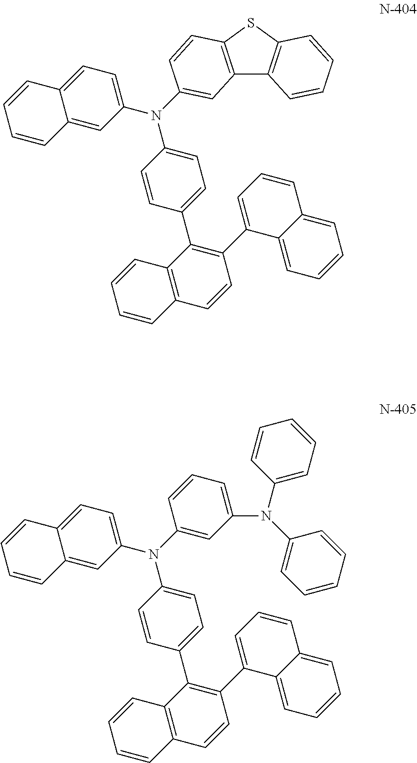 [SMILES: c1ccc2cc(N(c3ccc(-c4c(-c5cccc6ccccc56)ccc5ccccc45)cc3)c3ccc4sc5ccccc5c4c3)ccc2c1 ; c1ccc(N(c2ccccc2)c2cccc(N(c3ccc(-c4c(-c5cccc6ccccc56)ccc5ccccc45)cc3)c3ccc4ccccc4c3)c2)cc1]

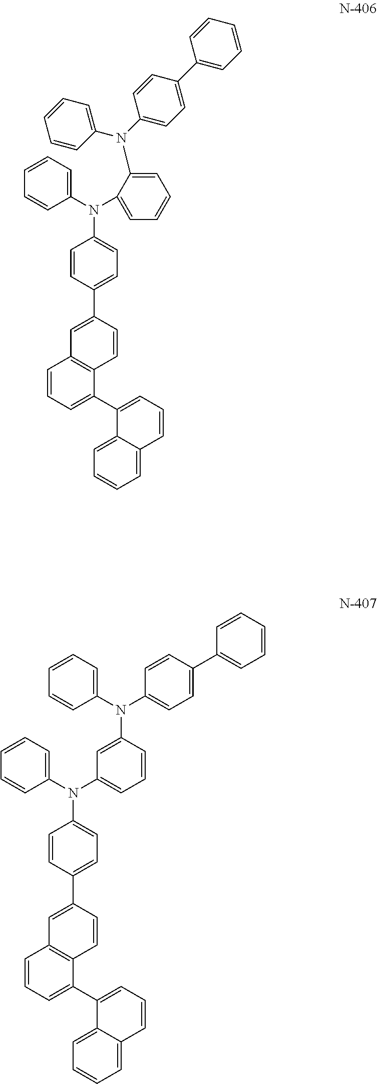 [SMILES: c1ccc(-c2ccc(N(c3ccccc3)c3ccccc3N(c3ccccc3)c3ccc(-c4ccc5c(-c6cccc7ccccc67)cccc5c4)cc3)cc2)cc1 ; c1ccc(-c2ccc(N(c3ccccc3)c3cccc(N(c4ccccc4)c4ccc(-c5ccc6c(-c7cccc8ccccc78)cccc6c5)cc4)c3)cc2)cc1]

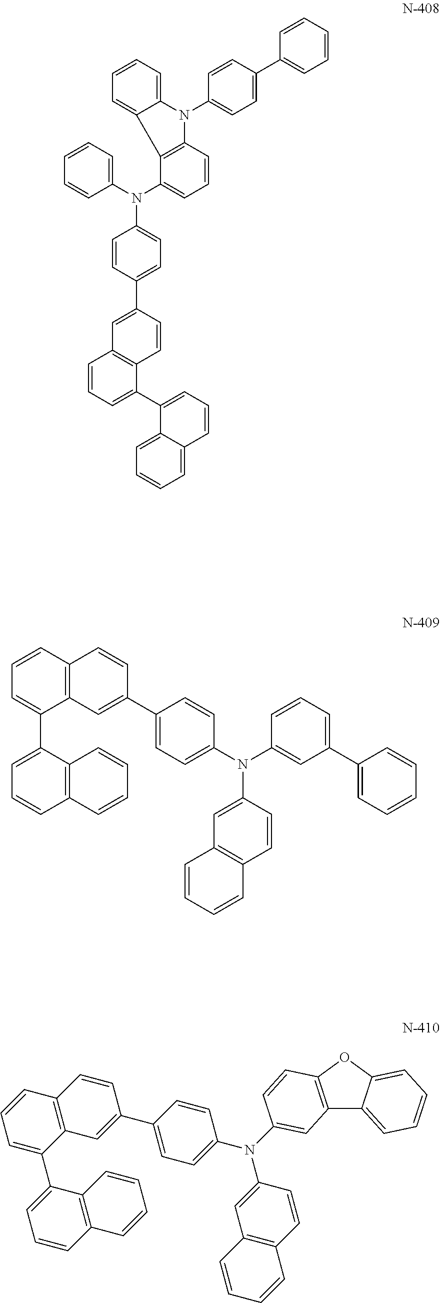 [SMILES: c1ccc(-c2ccc(-n3c4ccccc4c4c(N(c5ccccc5)c5ccc(-c6ccc7c(-c8cccc9ccccc89)cccc7c6)cc5)cccc43)cc2)cc1 ; c1ccc(-c2cccc(N(c3ccc(-c4ccc5cccc(-c6cccc7ccccc67)c5c4)cc3)c3ccc4ccccc4c3)c2)cc1 ; c1ccc2cc(N(c3ccc(-c4ccc5cccc(-c6cccc7ccccc67)c5c4)cc3)c3ccc4oc5ccccc5c4c3)ccc2c1]

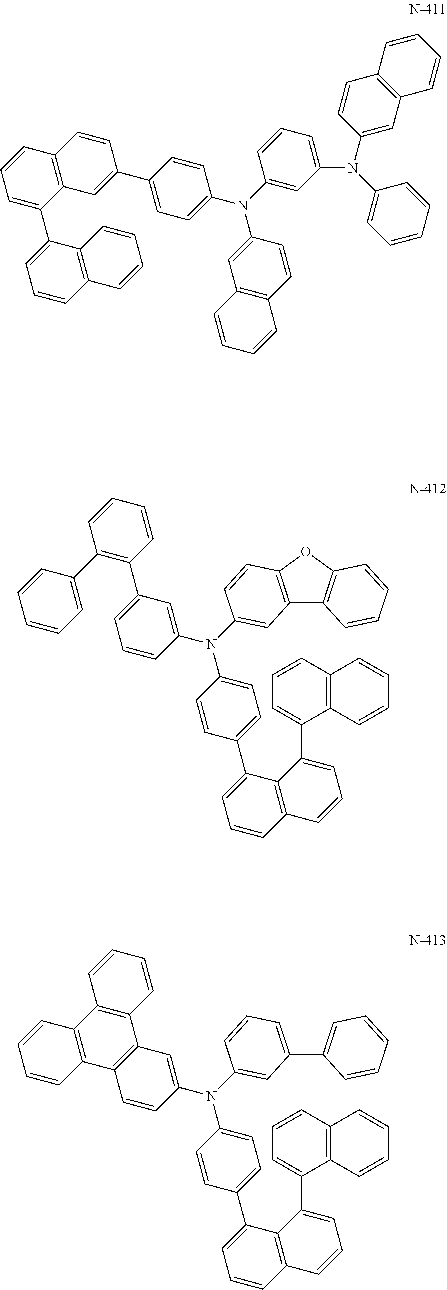 [SMILES: c1ccc(N(c2cccc(N(c3ccc(-c4ccc5cccc(-c6cccc7ccccc67)c5c4)cc3)c3ccc4ccccc4c3)c2)c2ccc3ccccc3c2)cc1 ; c1ccc(-c2ccccc2-c2cccc(N(c3ccc(-c4cccc5cccc(-c6cccc7ccccc67)c45)cc3)c3ccc4oc5ccccc5c4c3)c2)cc1 ; c1ccc(-c2cccc(N(c3ccc(-c4cccc5cccc(-c6cccc7ccccc67)c45)cc3)c3ccc4c5ccccc5c5ccccc5c4c3)c2)cc1]

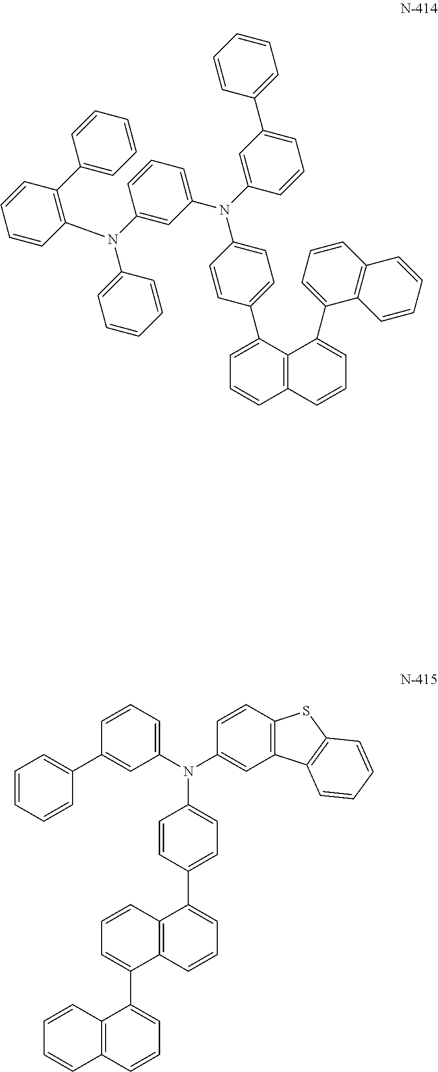 [SMILES: c1ccc(-c2cccc(N(c3ccc(-c4cccc5cccc(-c6cccc7ccccc67)c45)cc3)c3cccc(N(c4ccccc4)c4ccccc4-c4ccccc4)c3)c2)cc1 ; c1ccc(-c2cccc(N(c3ccc(-c4cccc5c(-c6cccc7ccccc67)cccc45)cc3)c3ccc4sc5ccccc5c4c3)c2)cc1]

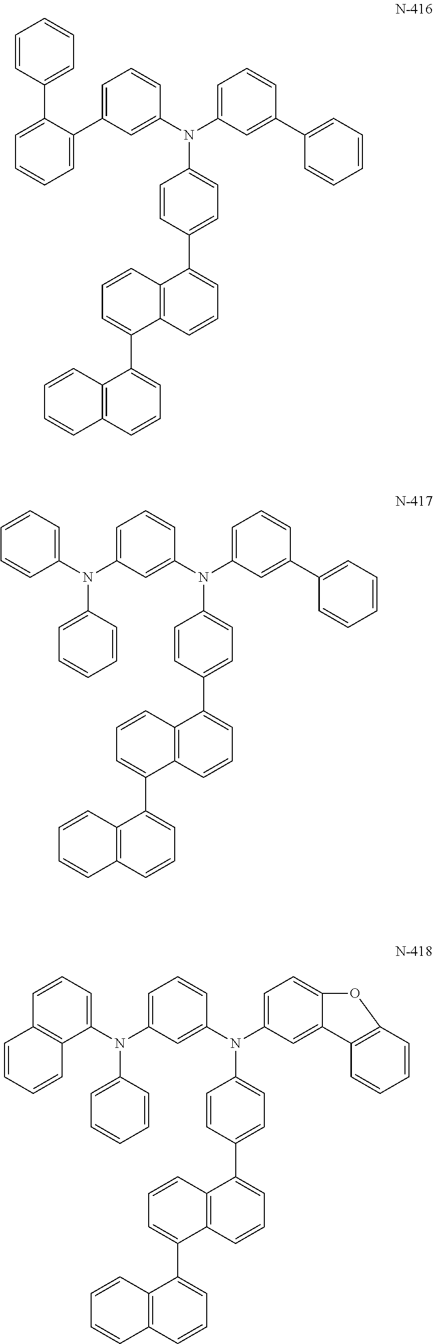 [SMILES: c1ccc(-c2cccc(N(c3ccc(-c4cccc5c(-c6cccc7ccccc67)cccc45)cc3)c3cccc(-c4ccccc4-c4ccccc4)c3)c2)cc1 ; c1ccc(-c2cccc(N(c3ccc(-c4cccc5c(-c6cccc7ccccc67)cccc45)cc3)c3cccc(N(c4ccccc4)c4ccccc4)c3)c2)cc1 ; c1ccc(N(c2cccc(N(c3ccc(-c4cccc5c(-c6cccc7ccccc67)cccc45)cc3)c3ccc4oc5ccccc5c4c3)c2)c2cccc3ccccc23)cc1]

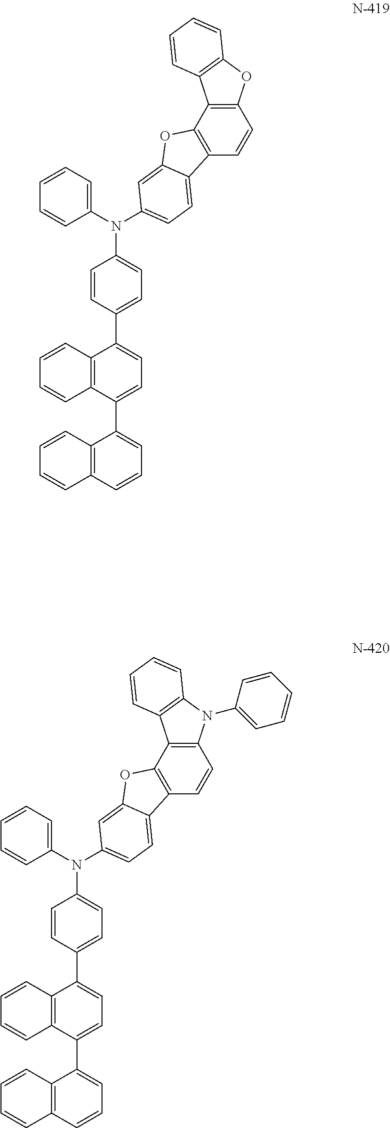 [SMILES: c1ccc(N(c2ccc(-c3ccc(-c4cccc5ccccc45)c4ccccc34)cc2)c2ccc3c(c2)oc2c3ccc3oc4ccccc4c32)cc1 ; c1ccc(N(c2ccc(-c3ccc(-c4cccc5ccccc45)c4ccccc34)cc2)c2ccc3c(c2)oc2c3ccc3c2c2ccccc2n3-c2ccccc2)cc1]

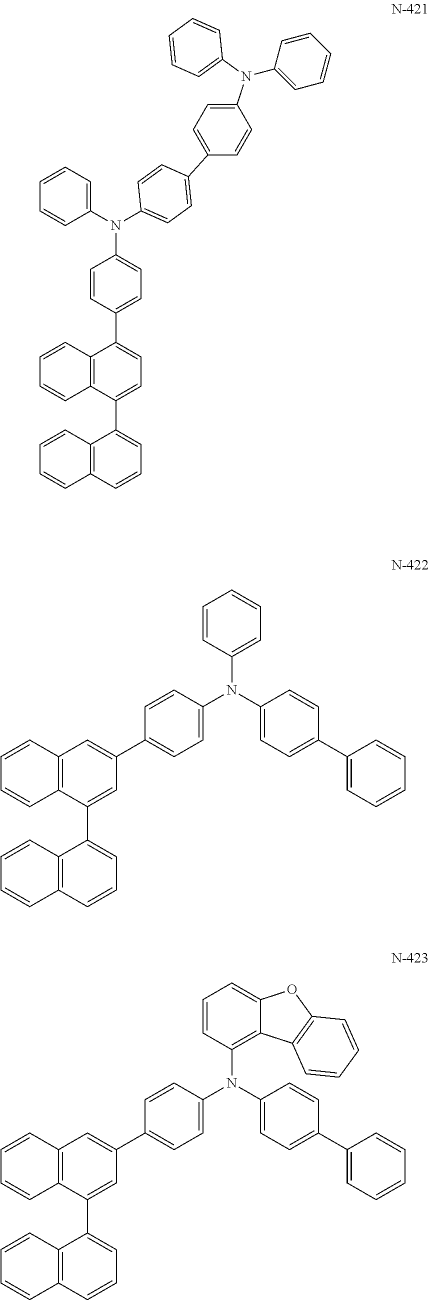 [SMILES: c1ccc(N(c2ccccc2)c2ccc(-c3ccc(N(c4ccccc4)c4ccc(-c5ccc(-c6cccc7ccccc67)c6ccccc56)cc4)cc3)cc2)cc1 ; c1ccc(-c2ccc(N(c3ccccc3)c3ccc(-c4cc(-c5cccc6ccccc56)c5ccccc5c4)cc3)cc2)cc1 ; c1ccc(-c2ccc(N(c3ccc(-c4cc(-c5cccc6ccccc56)c5ccccc5c4)cc3)c3cccc4oc5ccccc5c34)cc2)cc1]

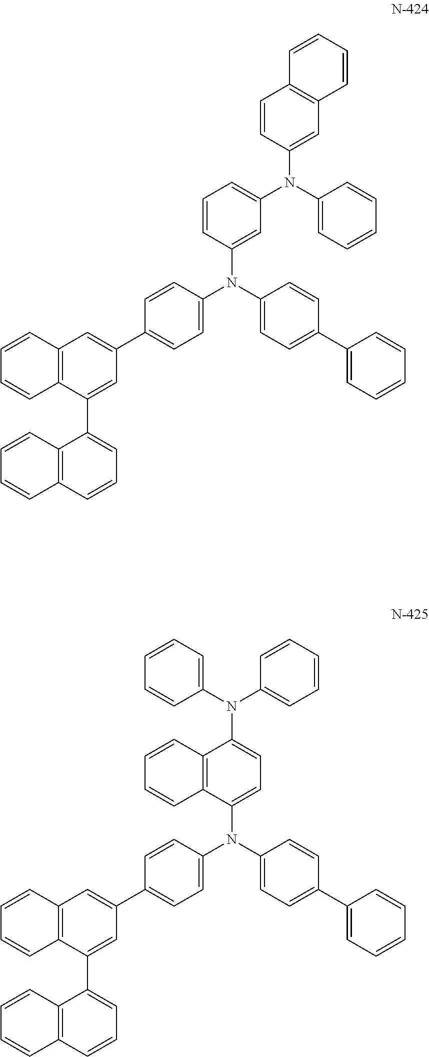 [SMILES: c1ccc(-c2ccc(N(c3ccc(-c4cc(-c5cccc6ccccc56)c5ccccc5c4)cc3)c3cccc(N(c4ccccc4)c4ccc5ccccc5c4)c3)cc2)cc1 ; c1ccc(-c2ccc(N(c3ccc(-c4cc(-c5cccc6ccccc56)c5ccccc5c4)cc3)c3ccc(N(c4ccccc4)c4ccccc4)c4ccccc34)cc2)cc1]

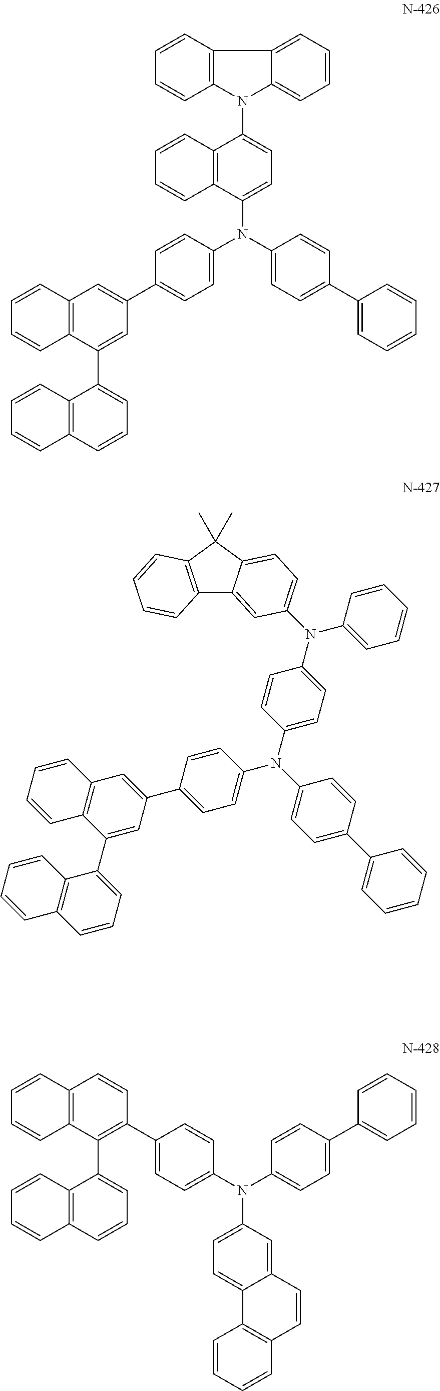 [SMILES: c1ccc(-c2ccc(N(c3ccc(-c4cc(-c5cccc6ccccc56)c5ccccc5c4)cc3)c3ccc(-n4c5ccccc5c5ccccc54)c4ccccc34)cc2)cc1 ; CC1(C)c2ccccc2-c2cc(N(c3ccccc3)c3ccc(N(c4ccc(-c5ccccc5)cc4)c4ccc(-c5cc(-c6cccc7ccccc67)c6ccccc6c5)cc4)cc3)ccc21 ; c1ccc(-c2ccc(N(c3ccc(-c4ccc5ccccc5c4-c4cccc5ccccc45)cc3)c3ccc4c(ccc5ccccc54)c3)cc2)cc1]

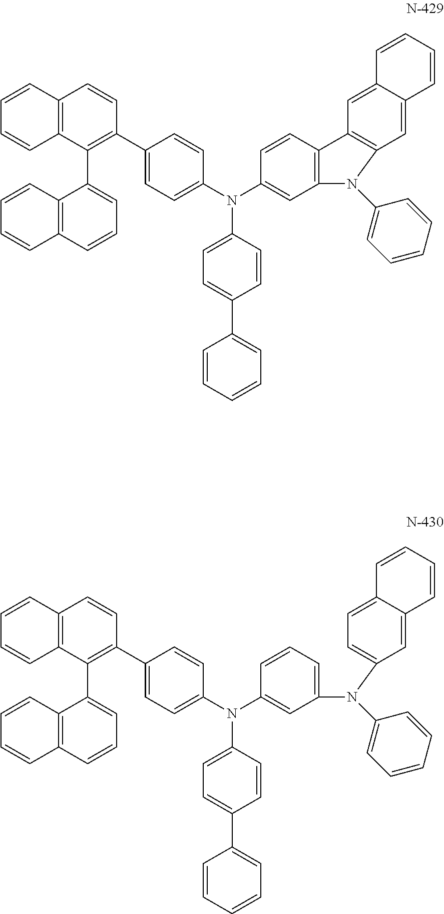 [SMILES: c1ccc(-c2ccc(N(c3ccc(-c4ccc5ccccc5c4-c4cccc5ccccc45)cc3)c3ccc4c5cc6ccccc6cc5n(-c5ccccc5)c4c3)cc2)cc1 ; c1ccc(-c2ccc(N(c3ccc(-c4ccc5ccccc5c4-c4cccc5ccccc45)cc3)c3cccc(N(c4ccccc4)c4ccc5ccccc5c4)c3)cc2)cc1]

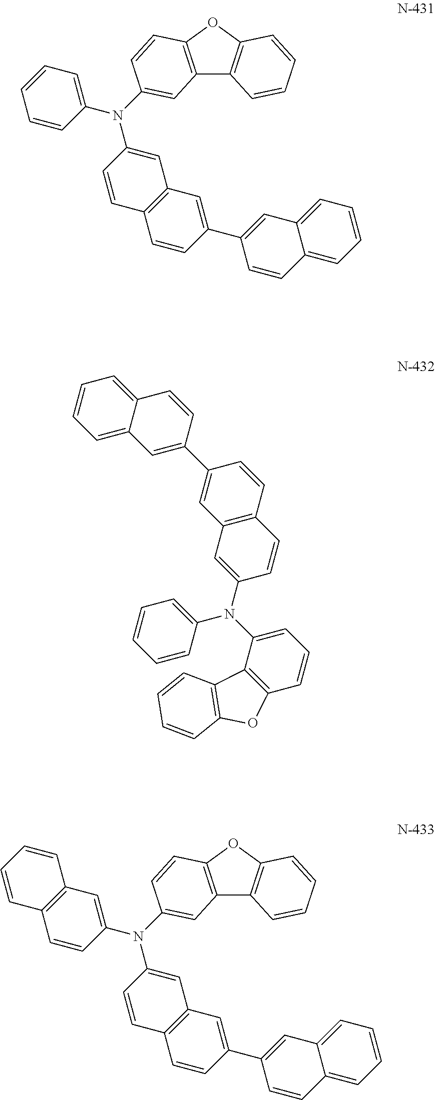 [SMILES: c1ccc(N(c2ccc3ccc(-c4ccc5ccccc5c4)cc3c2)c2ccc3oc4ccccc4c3c2)cc1 ; c1ccc(N(c2ccc3ccc(-c4ccc5ccccc5c4)cc3c2)c2cccc3oc4ccccc4c23)cc1 ; c1ccc2cc(-c3ccc4ccc(N(c5ccc6ccccc6c5)c5ccc6oc7ccccc7c6c5)cc4c3)ccc2c1]

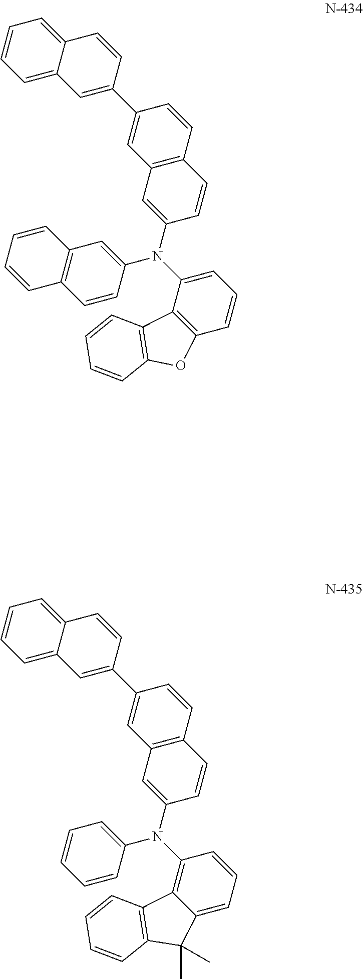 [SMILES: c1ccc2cc(-c3ccc4ccc(N(c5ccc6ccccc6c5)c5cccc6oc7ccccc7c56)cc4c3)ccc2c1 ; CC1(C)c2ccccc2-c2c(N(c3ccccc3)c3ccc4ccc(-c5ccc6ccccc6c5)cc4c3)cccc21]

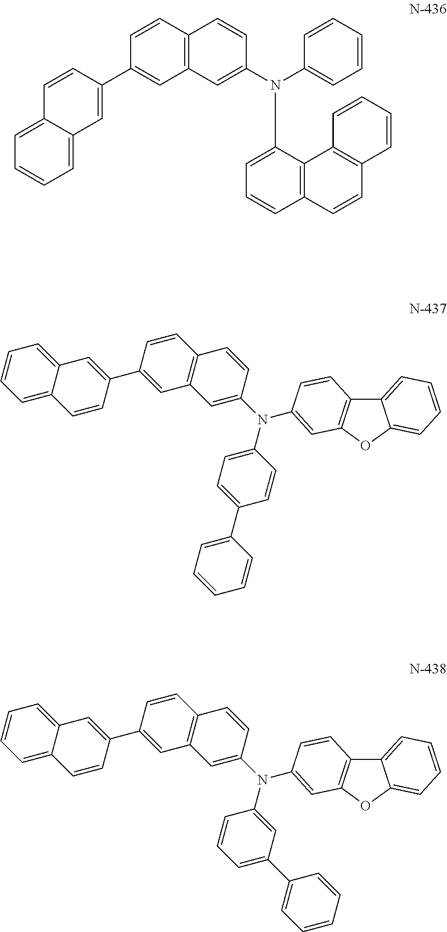 [SMILES: c1ccc(N(c2ccc3ccc(-c4ccc5ccccc5c4)cc3c2)c2cccc3ccc4ccccc4c23)cc1 ; c1ccc(-c2ccc(N(c3ccc4ccc(-c5ccc6ccccc6c5)cc4c3)c3ccc4c(c3)oc3ccccc34)cc2)cc1 ; c1ccc(-c2cccc(N(c3ccc4ccc(-c5ccc6ccccc6c5)cc4c3)c3ccc4c(c3)oc3ccccc34)c2)cc1]

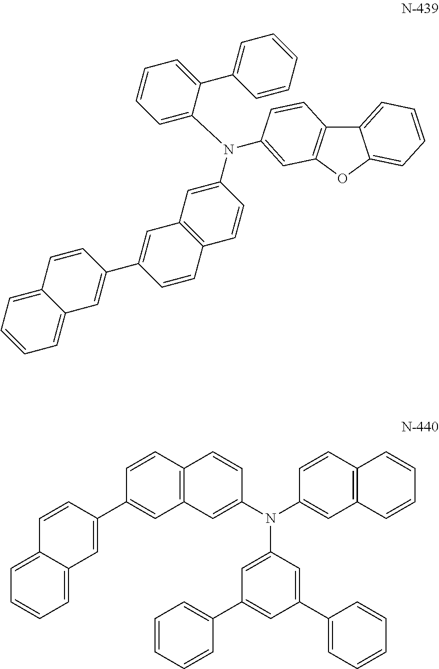 [SMILES: c1ccc(-c2ccccc2N(c2ccc3ccc(-c4ccc5ccccc5c4)cc3c2)c2ccc3c(c2)oc2ccccc23)cc1 ; c1ccc(-c2cc(-c3ccccc3)cc(N(c3ccc4ccccc4c3)c3ccc4ccc(-c5ccc6ccccc6c5)cc4c3)c2)cc1]

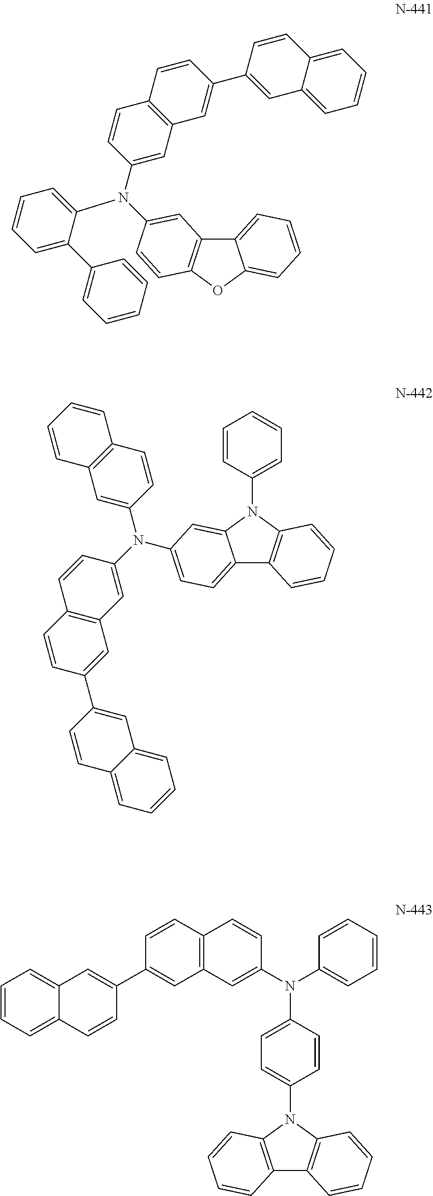 [SMILES: c1ccc(-c2ccccc2N(c2ccc3ccc(-c4ccc5ccccc5c4)cc3c2)c2ccc3oc4ccccc4c3c2)cc1 ; c1ccc(-n2c3ccccc3c3ccc(N(c4ccc5ccccc5c4)c4ccc5ccc(-c6ccc7ccccc7c6)cc5c4)cc32)cc1 ; c1ccc(N(c2ccc(-n3c4ccccc4c4ccccc43)cc2)c2ccc3ccc(-c4ccc5ccccc5c4)cc3c2)cc1]

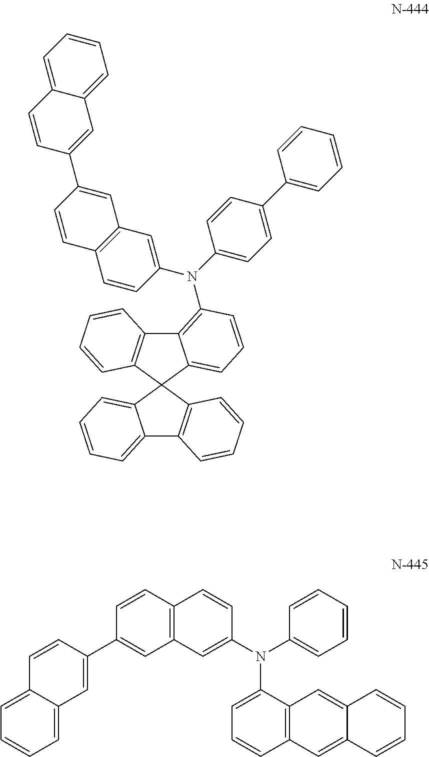 [SMILES: c1ccc(-c2ccc(N(c3ccc4ccc(-c5ccc6ccccc6c5)cc4c3)c3cccc4c3-c3ccccc3C43c4ccccc4-c4ccccc43)cc2)cc1 ; c1ccc(N(c2ccc3ccc(-c4ccc5ccccc5c4)cc3c2)c2cccc3cc4ccccc4cc23)cc1]

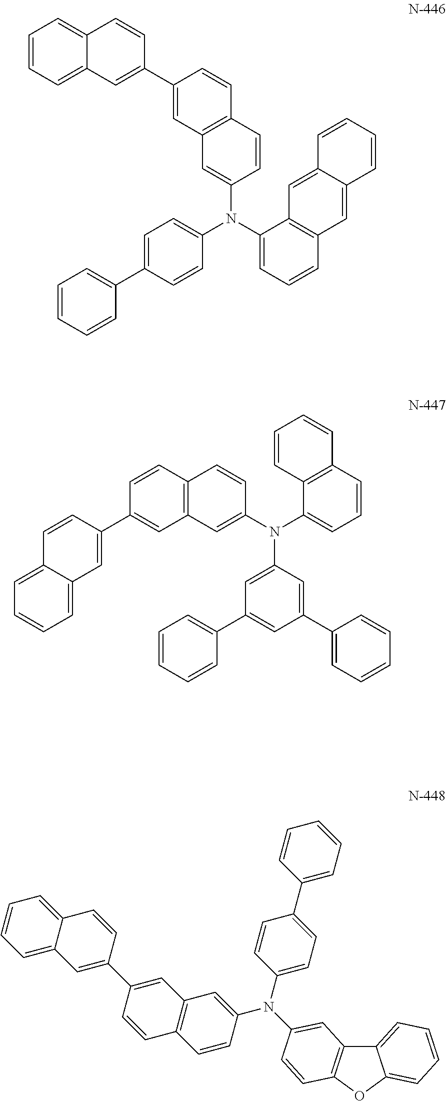 [SMILES: c1ccc(-c2ccc(N(c3ccc4ccc(-c5ccc6ccccc6c5)cc4c3)c3cccc4cc5ccccc5cc34)cc2)cc1 ; c1ccc(-c2cc(-c3ccccc3)cc(N(c3ccc4ccc(-c5ccc6ccccc6c5)cc4c3)c3cccc4ccccc34)c2)cc1 ; c1ccc(-c2ccc(N(c3ccc4ccc(-c5ccc6ccccc6c5)cc4c3)c3ccc4oc5ccccc5c4c3)cc2)cc1]

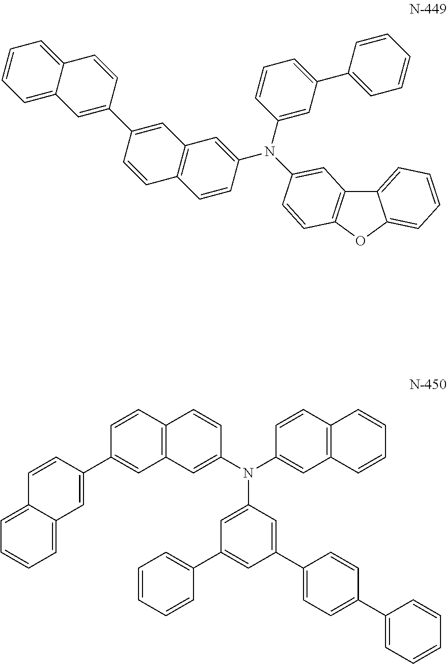 [SMILES: c1ccc(-c2cccc(N(c3ccc4ccc(-c5ccc6ccccc6c5)cc4c3)c3ccc4oc5ccccc5c4c3)c2)cc1 ; c1ccc(-c2ccc(-c3cc(-c4ccccc4)cc(N(c4ccc5ccccc5c4)c4ccc5ccc(-c6ccc7ccccc7c6)cc5c4)c3)cc2)cc1]

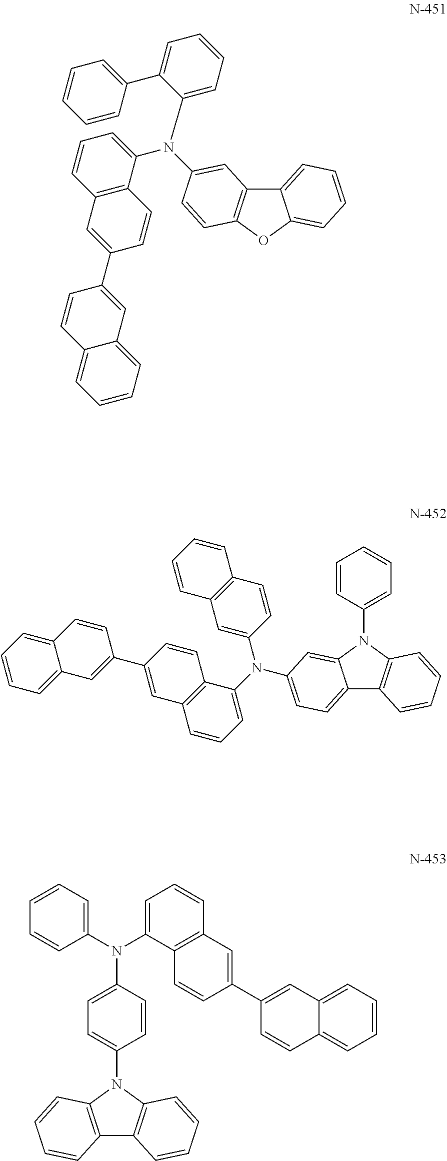 [SMILES: c1ccc(-c2ccccc2N(c2ccc3oc4ccccc4c3c2)c2cccc3cc(-c4ccc5ccccc5c4)ccc23)cc1 ; c1ccc(-n2c3ccccc3c3ccc(N(c4ccc5ccccc5c4)c4cccc5cc(-c6ccc7ccccc7c6)ccc45)cc32)cc1 ; c1ccc(N(c2ccc(-n3c4ccccc4c4ccccc43)cc2)c2cccc3cc(-c4ccc5ccccc5c4)ccc23)cc1]

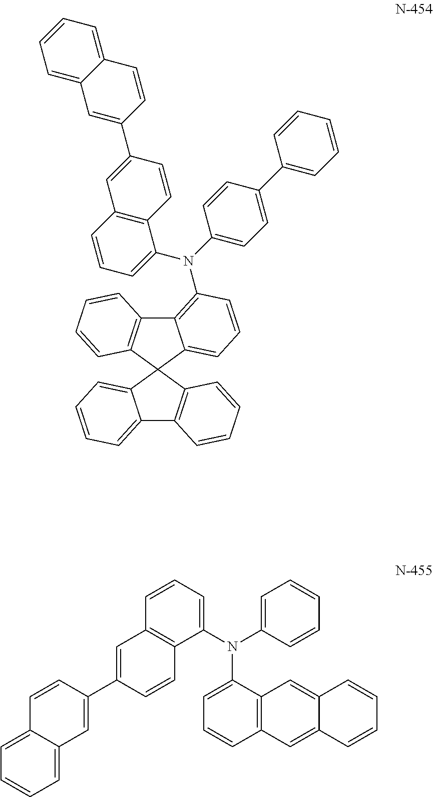 [SMILES: c1ccc(-c2ccc(N(c3cccc4c3-c3ccccc3C43c4ccccc4-c4ccccc43)c3cccc4cc(-c5ccc6ccccc6c5)ccc34)cc2)cc1 ; c1ccc(N(c2cccc3cc(-c4ccc5ccccc5c4)ccc23)c2cccc3cc4ccccc4cc23)cc1]

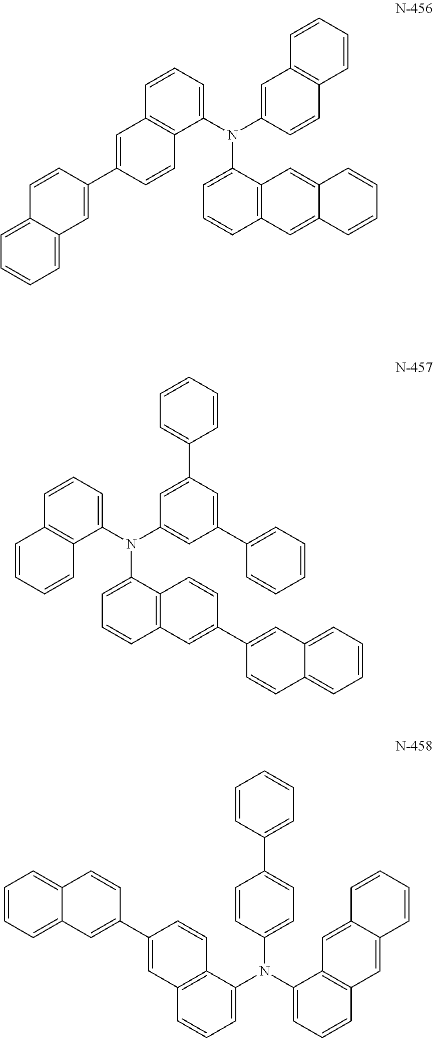 [SMILES: c1ccc2cc(-c3ccc4c(N(c5ccc6ccccc6c5)c5cccc6cc7ccccc7cc56)cccc4c3)ccc2c1 ; c1ccc(-c2cc(-c3ccccc3)cc(N(c3cccc4ccccc34)c3cccc4cc(-c5ccc6ccccc6c5)ccc34)c2)cc1 ; c1ccc(-c2ccc(N(c3cccc4cc(-c5ccc6ccccc6c5)ccc34)c3cccc4cc5ccccc5cc34)cc2)cc1]

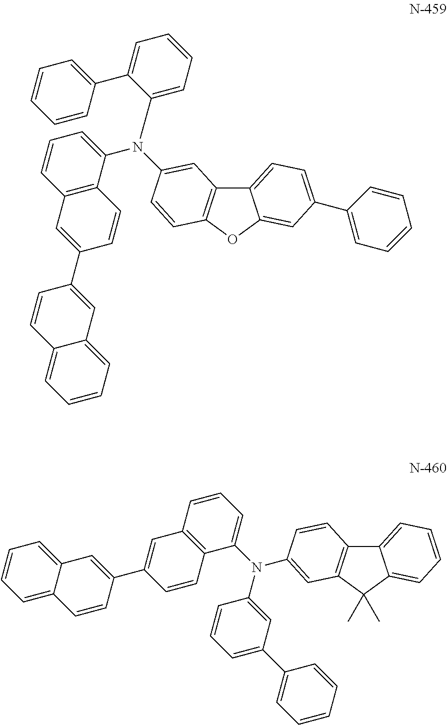 [SMILES: c1ccc(-c2ccc3c(c2)oc2ccc(N(c4ccccc4-c4ccccc4)c4cccc5cc(-c6ccc7ccccc7c6)ccc45)cc23)cc1 ; CC1(C)c2ccccc2-c2ccc(N(c3cccc(-c4ccccc4)c3)c3cccc4cc(-c5ccc6ccccc6c5)ccc34)cc21]

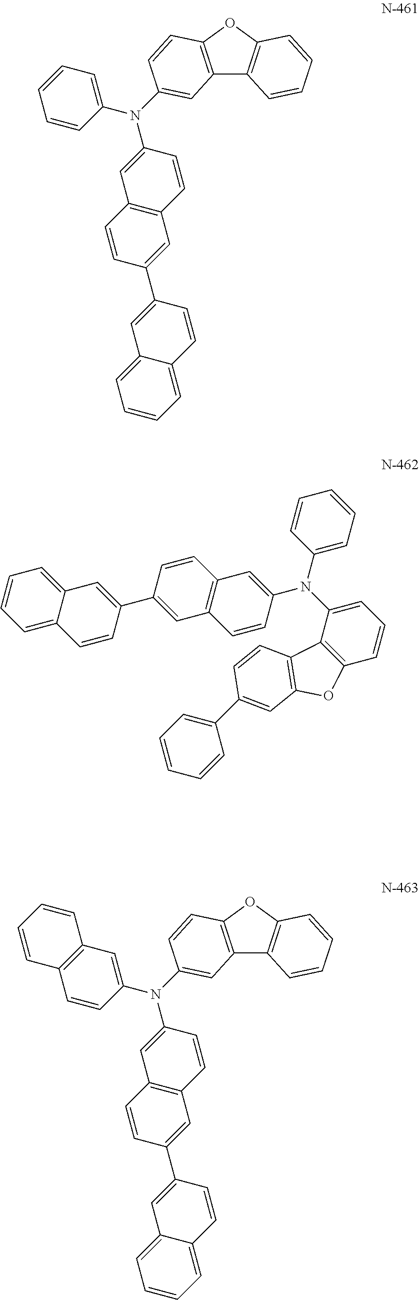 [SMILES: c1ccc(N(c2ccc3cc(-c4ccc5ccccc5c4)ccc3c2)c2ccc3oc4ccccc4c3c2)cc1 ; c1ccc(-c2ccc3c(c2)oc2cccc(N(c4ccccc4)c4ccc5cc(-c6ccc7ccccc7c6)ccc5c4)c23)cc1 ; c1ccc2cc(-c3ccc4cc(N(c5ccc6ccccc6c5)c5ccc6oc7ccccc7c6c5)ccc4c3)ccc2c1]

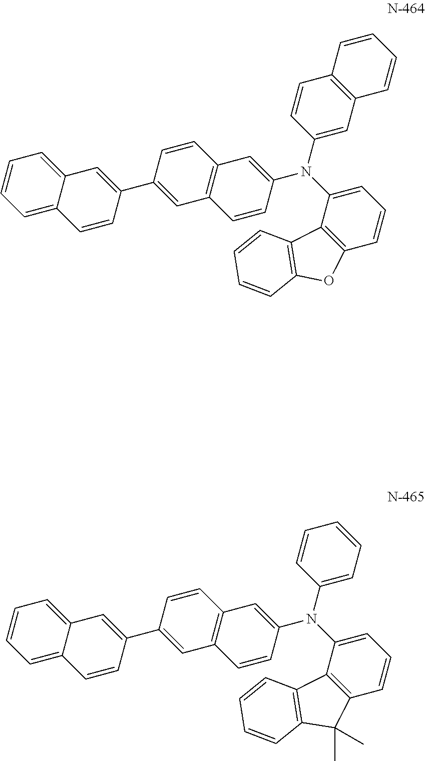 [SMILES: c1ccc2cc(-c3ccc4cc(N(c5ccc6ccccc6c5)c5cccc6oc7ccccc7c56)ccc4c3)ccc2c1 ; CC1(C)c2ccccc2-c2c(N(c3ccccc3)c3ccc4cc(-c5ccc6ccccc6c5)ccc4c3)cccc21]

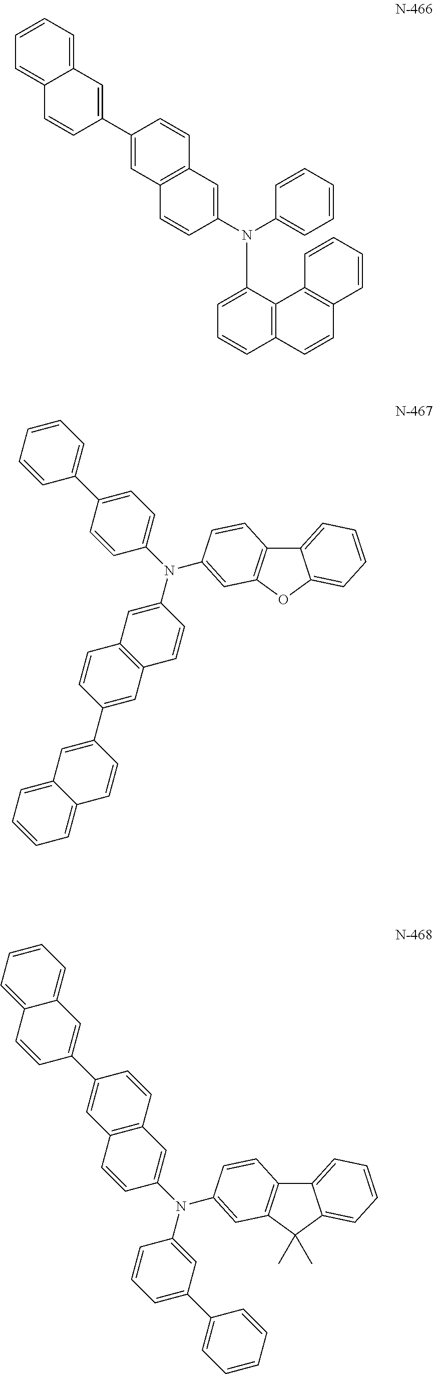 [SMILES: c1ccc(N(c2ccc3cc(-c4ccc5ccccc5c4)ccc3c2)c2cccc3ccc4ccccc4c23)cc1 ; c1ccc(-c2ccc(N(c3ccc4cc(-c5ccc6ccccc6c5)ccc4c3)c3ccc4c(c3)oc3ccccc34)cc2)cc1 ; CC1(C)c2ccccc2-c2ccc(N(c3cccc(-c4ccccc4)c3)c3ccc4cc(-c5ccc6ccccc6c5)ccc4c3)cc21]

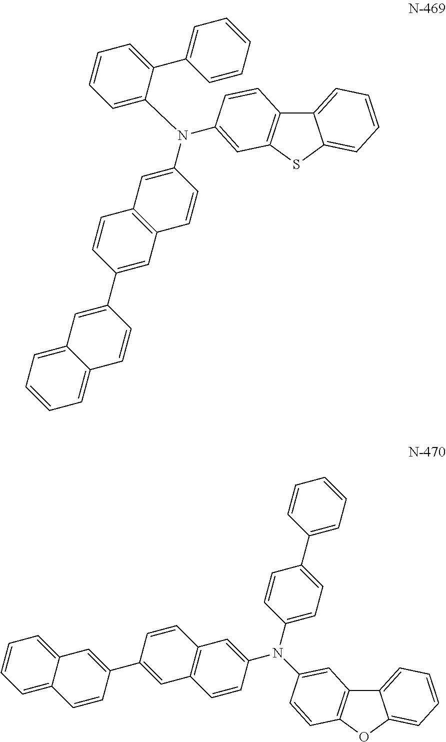 [SMILES: c1ccc(-c2ccccc2N(c2ccc3cc(-c4ccc5ccccc5c4)ccc3c2)c2ccc3c(c2)sc2ccccc23)cc1 ; c1ccc(-c2ccc(N(c3ccc4cc(-c5ccc6ccccc6c5)ccc4c3)c3ccc4oc5ccccc5c4c3)cc2)cc1]

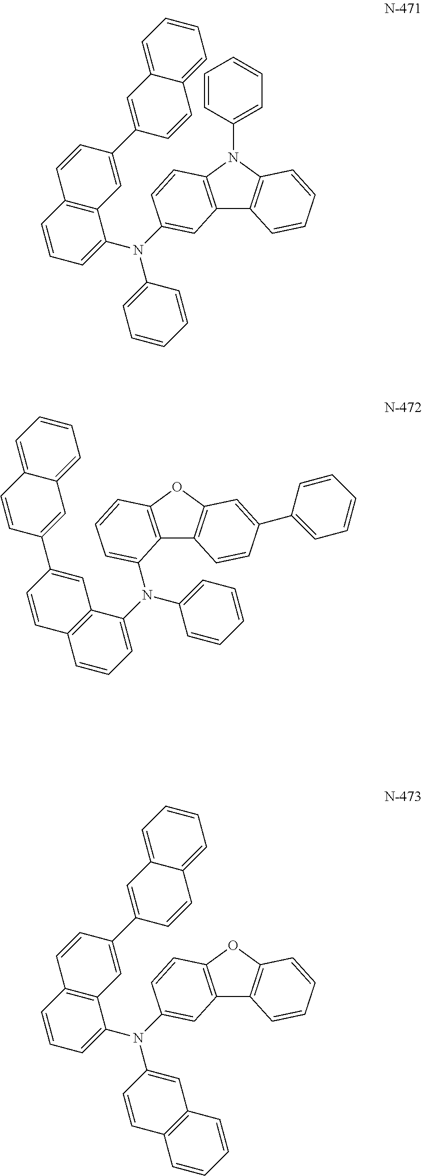 [SMILES: c1ccc(N(c2ccc3c(c2)c2ccccc2n3-c2ccccc2)c2cccc3ccc(-c4ccc5ccccc5c4)cc23)cc1 ; c1ccc(-c2ccc3c(c2)oc2cccc(N(c4ccccc4)c4cccc5ccc(-c6ccc7ccccc7c6)cc45)c23)cc1 ; c1ccc2cc(-c3ccc4cccc(N(c5ccc6ccccc6c5)c5ccc6oc7ccccc7c6c5)c4c3)ccc2c1]

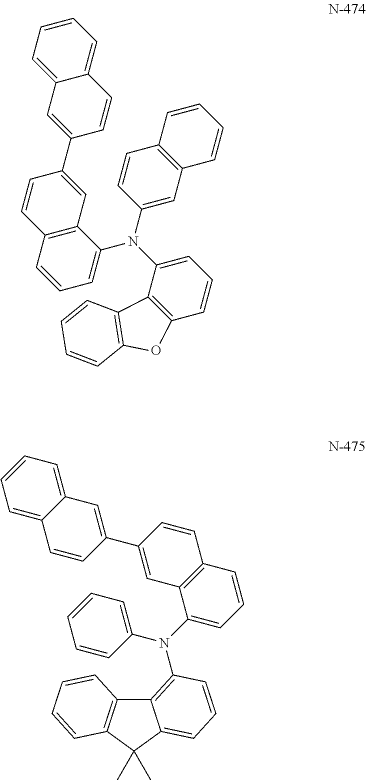 [SMILES: c1ccc2cc(-c3ccc4cccc(N(c5ccc6ccccc6c5)c5cccc6oc7ccccc7c56)c4c3)ccc2c1 ; CC1(C)c2ccccc2-c2c(N(c3ccccc3)c3cccc4ccc(-c5ccc6ccccc6c5)cc34)cccc21]

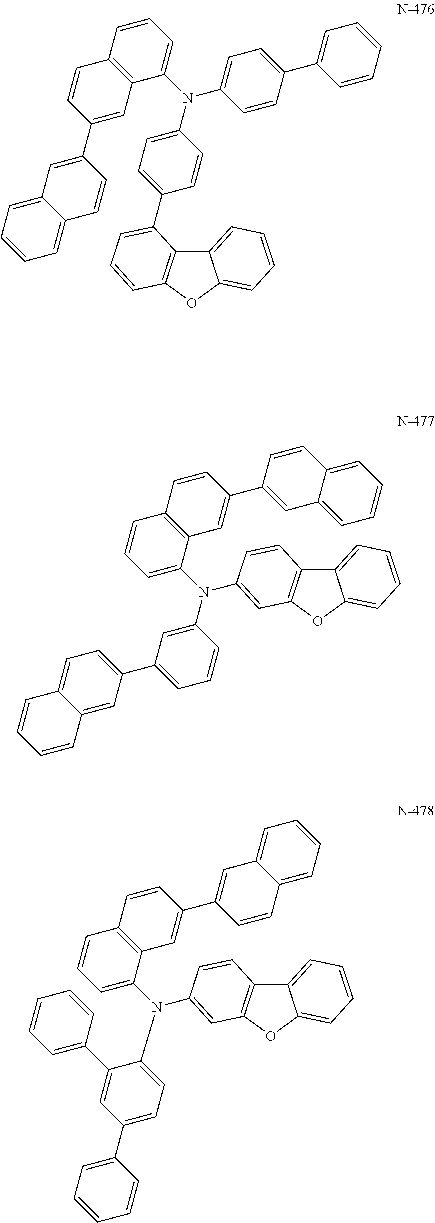 [SMILES: c1ccc(-c2ccc(N(c3ccc(-c4cccc5oc6ccccc6c45)cc3)c3cccc4ccc(-c5ccc6ccccc6c5)cc34)cc2)cc1 ; c1cc(-c2ccc3ccccc3c2)cc(N(c2ccc3c(c2)oc2ccccc23)c2cccc3ccc(-c4ccc5ccccc5c4)cc23)c1 ; c1ccc(-c2ccc(N(c3ccc4c(c3)oc3ccccc34)c3cccc4ccc(-c5ccc6ccccc6c5)cc34)c(-c3ccccc3)c2)cc1]

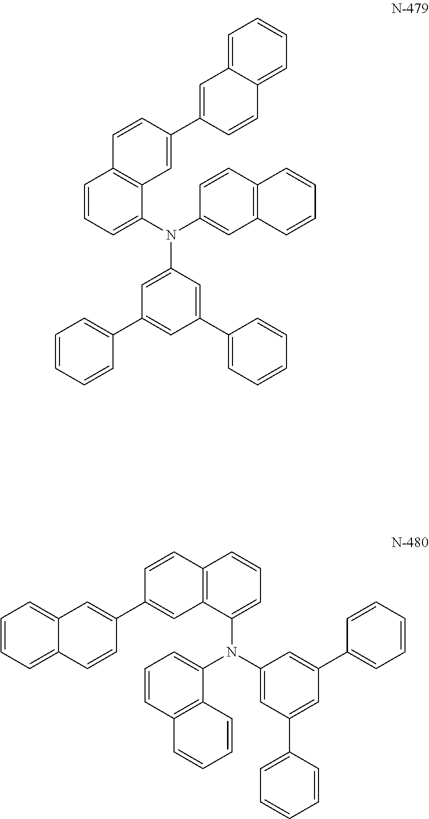 [SMILES: c1ccc(-c2cc(-c3ccccc3)cc(N(c3ccc4ccccc4c3)c3cccc4ccc(-c5ccc6ccccc6c5)cc34)c2)cc1 ; c1ccc(-c2cc(-c3ccccc3)cc(N(c3cccc4ccccc34)c3cccc4ccc(-c5ccc6ccccc6c5)cc34)c2)cc1]

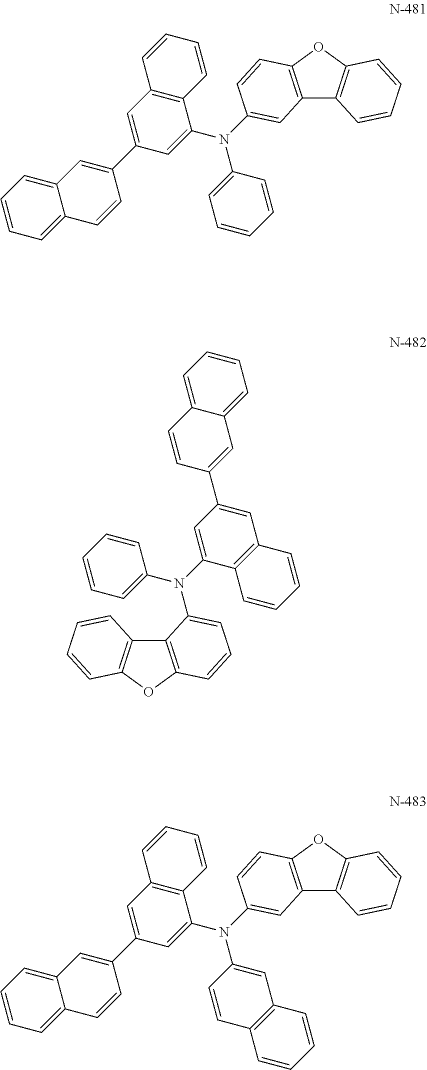 [SMILES: c1ccc(N(c2ccc3oc4ccccc4c3c2)c2cc(-c3ccc4ccccc4c3)cc3ccccc23)cc1 ; c1ccc(N(c2cc(-c3ccc4ccccc4c3)cc3ccccc23)c2cccc3oc4ccccc4c23)cc1 ; c1ccc2cc(-c3cc(N(c4ccc5ccccc5c4)c4ccc5oc6ccccc6c5c4)c4ccccc4c3)ccc2c1]

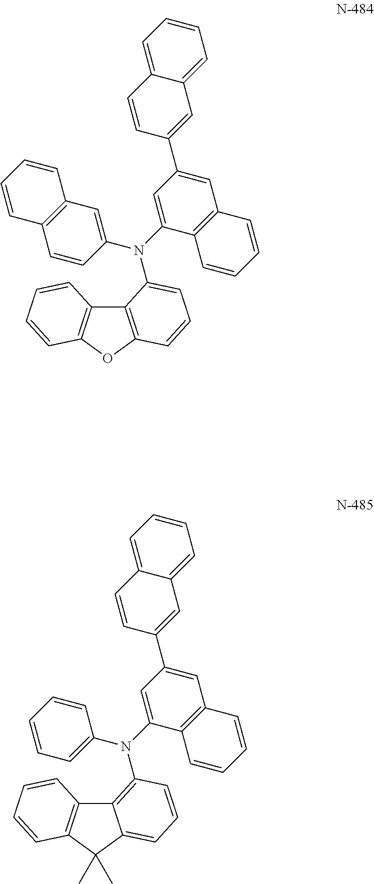 [SMILES: c1ccc2cc(-c3cc(N(c4ccc5ccccc5c4)c4cccc5oc6ccccc6c45)c4ccccc4c3)ccc2c1 ; CC1(C)c2ccccc2-c2c(N(c3ccccc3)c3cc(-c4ccc5ccccc5c4)cc4ccccc34)cccc21]

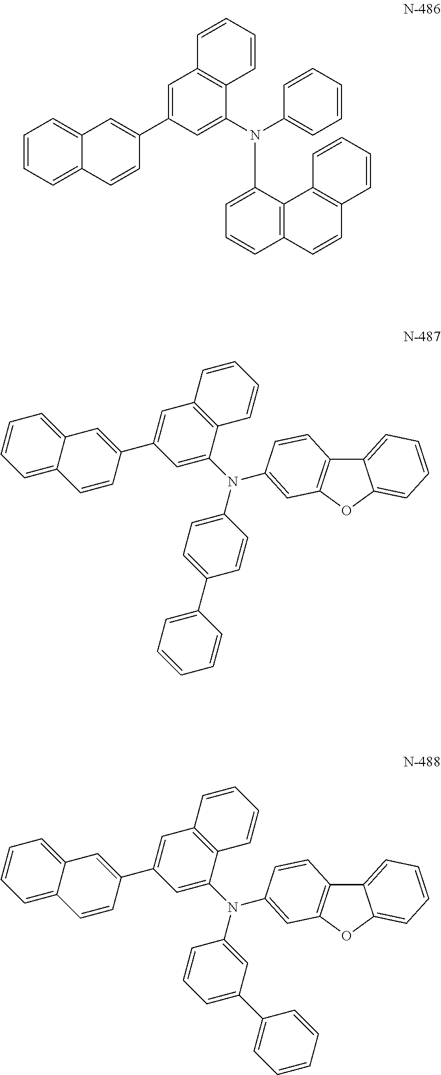 [SMILES: c1ccc(N(c2cc(-c3ccc4ccccc4c3)cc3ccccc23)c2cccc3ccc4ccccc4c23)cc1 ; c1ccc(-c2ccc(N(c3ccc4c(c3)oc3ccccc34)c3cc(-c4ccc5ccccc5c4)cc4ccccc34)cc2)cc1 ; c1ccc(-c2cccc(N(c3ccc4c(c3)oc3ccccc34)c3cc(-c4ccc5ccccc5c4)cc4ccccc34)c2)cc1]

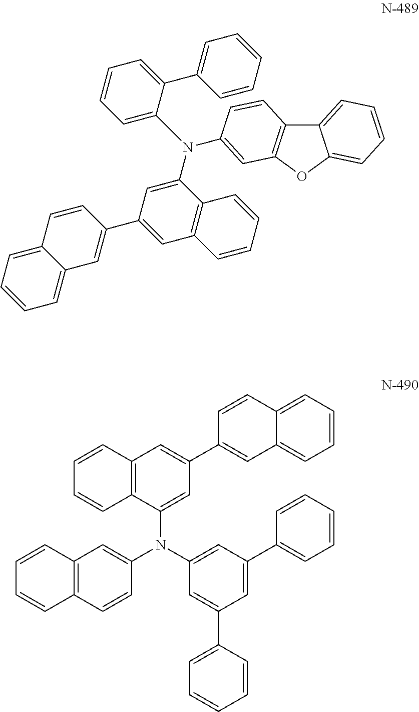 [SMILES: c1ccc(-c2ccccc2N(c2ccc3c(c2)oc2ccccc23)c2cc(-c3ccc4ccccc4c3)cc3ccccc23)cc1 ; c1ccc(-c2cc(-c3ccccc3)cc(N(c3ccc4ccccc4c3)c3cc(-c4ccc5ccccc5c4)cc4ccccc34)c2)cc1]

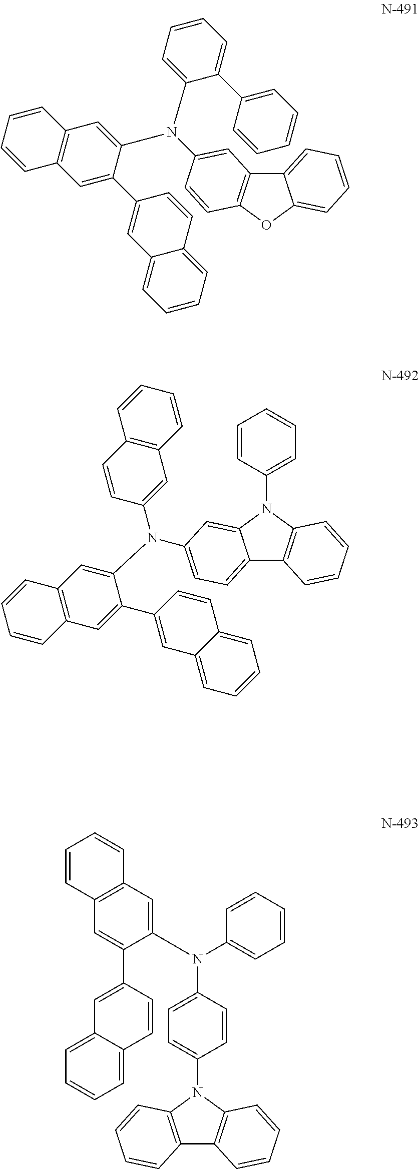 [SMILES: c1ccc(-c2ccccc2N(c2ccc3oc4ccccc4c3c2)c2cc3ccccc3cc2-c2ccc3ccccc3c2)cc1 ; c1ccc(-n2c3ccccc3c3ccc(N(c4ccc5ccccc5c4)c4cc5ccccc5cc4-c4ccc5ccccc5c4)cc32)cc1 ; c1ccc(N(c2ccc(-n3c4ccccc4c4ccccc43)cc2)c2cc3ccccc3cc2-c2ccc3ccccc3c2)cc1]

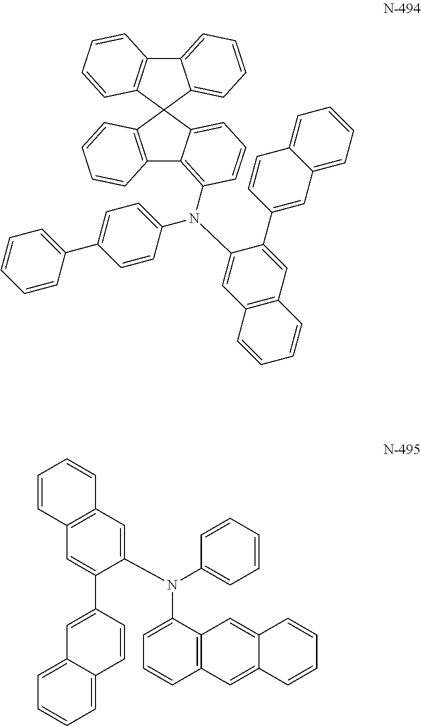 [SMILES: c1ccc(-c2ccc(N(c3cc4ccccc4cc3-c3ccc4ccccc4c3)c3cccc4c3-c3ccccc3C43c4ccccc4-c4ccccc43)cc2)cc1 ; c1ccc(N(c2cc3ccccc3cc2-c2ccc3ccccc3c2)c2cccc3cc4ccccc4cc23)cc1]

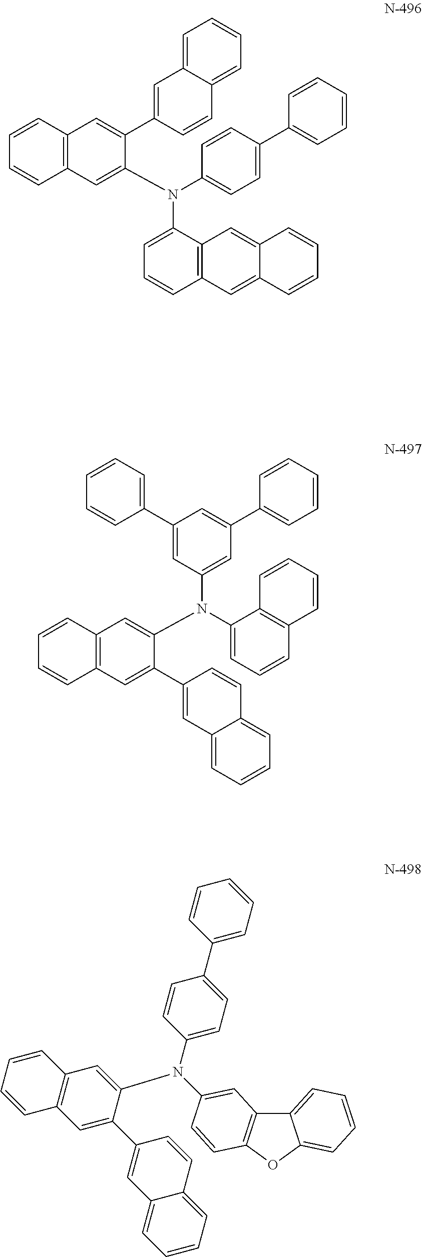 [SMILES: c1ccc(-c2ccc(N(c3cc4ccccc4cc3-c3ccc4ccccc4c3)c3cccc4cc5ccccc5cc34)cc2)cc1 ; c1ccc(-c2cc(-c3ccccc3)cc(N(c3cc4ccccc4cc3-c3ccc4ccccc4c3)c3cccc4ccccc34)c2)cc1 ; c1ccc(-c2ccc(N(c3ccc4oc5ccccc5c4c3)c3cc4ccccc4cc3-c3ccc4ccccc4c3)cc2)cc1]

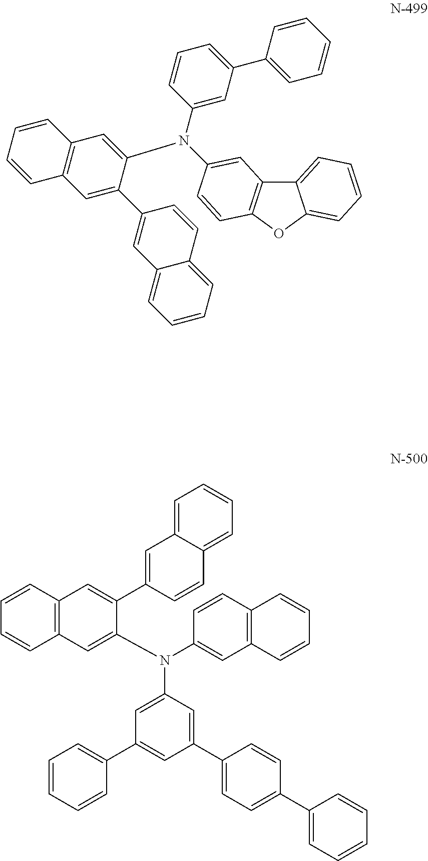 [SMILES: c1ccc(-c2cccc(N(c3ccc4oc5ccccc5c4c3)c3cc4ccccc4cc3-c3ccc4ccccc4c3)c2)cc1 ; c1ccc(-c2ccc(-c3cc(-c4ccccc4)cc(N(c4ccc5ccccc5c4)c4cc5ccccc5cc4-c4ccc5ccccc5c4)c3)cc2)cc1]

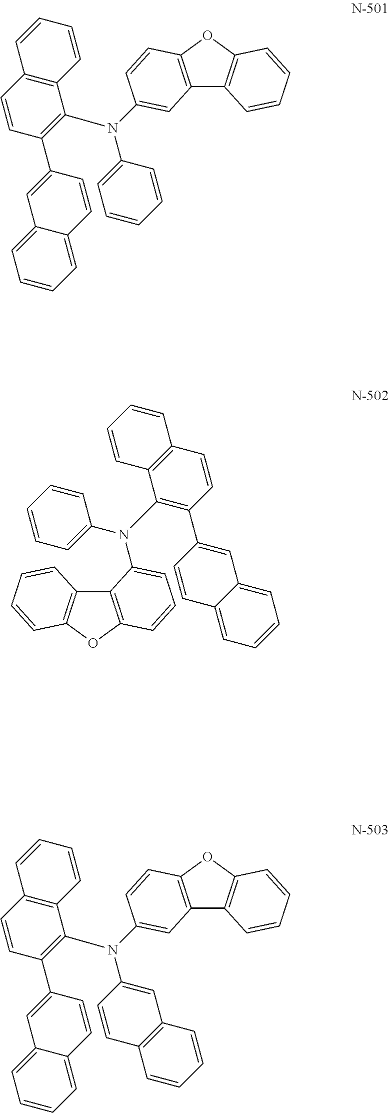 [SMILES: c1ccc(N(c2ccc3oc4ccccc4c3c2)c2c(-c3ccc4ccccc4c3)ccc3ccccc23)cc1 ; c1ccc(N(c2c(-c3ccc4ccccc4c3)ccc3ccccc23)c2cccc3oc4ccccc4c23)cc1 ; c1ccc2cc(-c3ccc4ccccc4c3N(c3ccc4ccccc4c3)c3ccc4oc5ccccc5c4c3)ccc2c1]

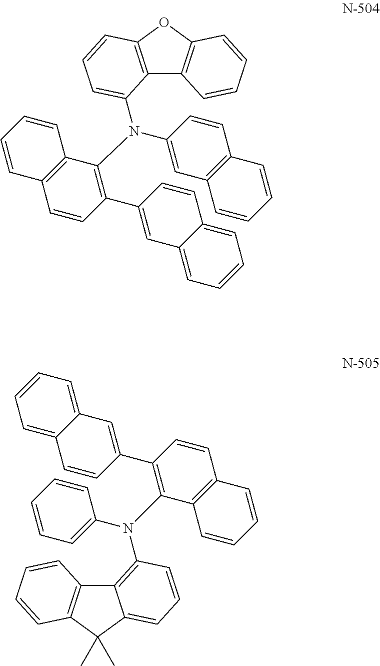 [SMILES: c1ccc2cc(-c3ccc4ccccc4c3N(c3ccc4ccccc4c3)c3cccc4oc5ccccc5c34)ccc2c1 ; CC1(C)c2ccccc2-c2c(N(c3ccccc3)c3c(-c4ccc5ccccc5c4)ccc4ccccc34)cccc21]

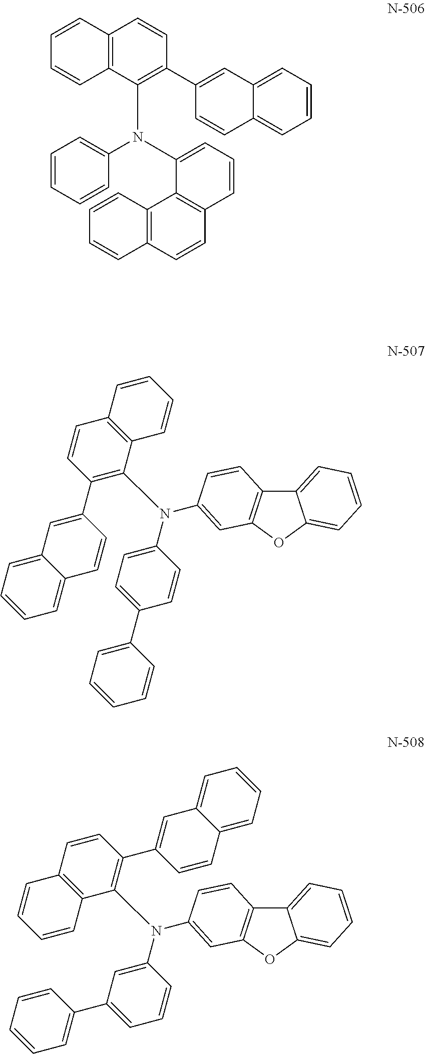 [SMILES: c1ccc(N(c2c(-c3ccc4ccccc4c3)ccc3ccccc23)c2cccc3ccc4ccccc4c23)cc1 ; c1ccc(-c2ccc(N(c3ccc4c(c3)oc3ccccc34)c3c(-c4ccc5ccccc5c4)ccc4ccccc34)cc2)cc1 ; c1ccc(-c2cccc(N(c3ccc4c(c3)oc3ccccc34)c3c(-c4ccc5ccccc5c4)ccc4ccccc34)c2)cc1]

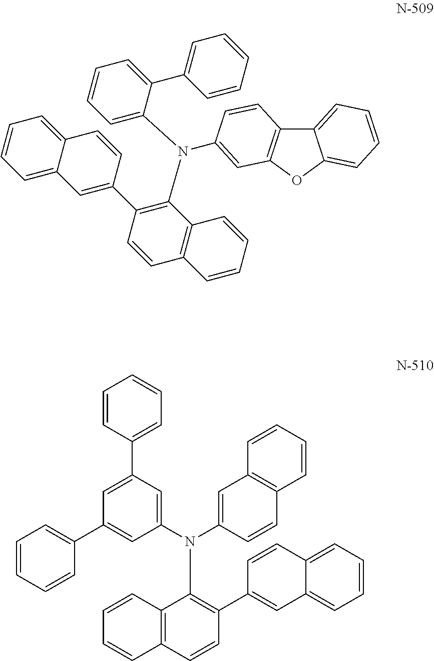 [SMILES: c1ccc(-c2ccccc2N(c2ccc3c(c2)oc2ccccc23)c2c(-c3ccc4ccccc4c3)ccc3ccccc23)cc1 ; c1ccc(-c2cc(-c3ccccc3)cc(N(c3ccc4ccccc4c3)c3c(-c4ccc5ccccc5c4)ccc4ccccc34)c2)cc1]

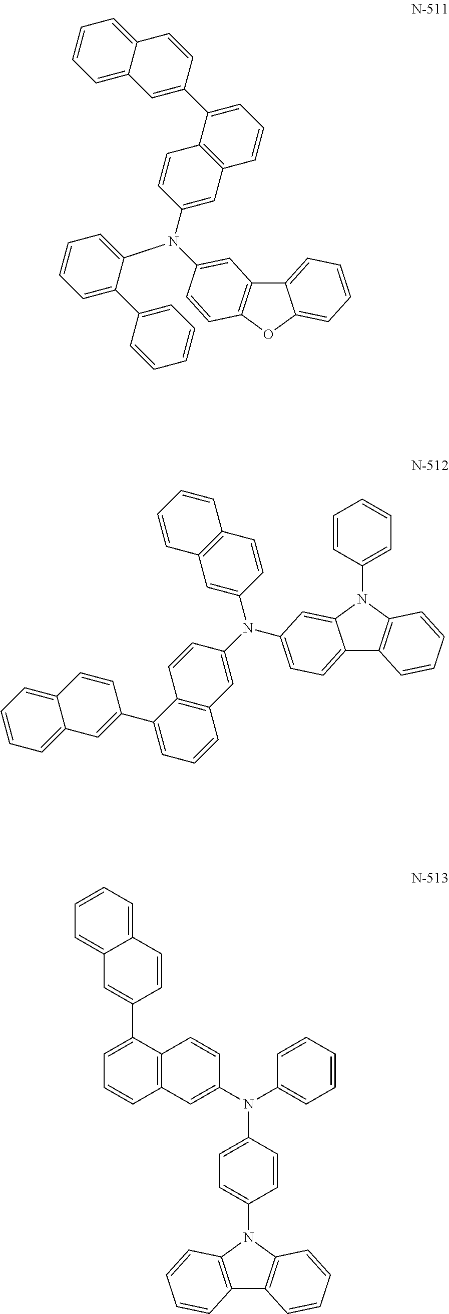 [SMILES: c1ccc(-c2ccccc2N(c2ccc3c(-c4ccc5ccccc5c4)cccc3c2)c2ccc3oc4ccccc4c3c2)cc1 ; c1ccc(-n2c3ccccc3c3ccc(N(c4ccc5ccccc5c4)c4ccc5c(-c6ccc7ccccc7c6)cccc5c4)cc32)cc1 ; c1ccc(N(c2ccc(-n3c4ccccc4c4ccccc43)cc2)c2ccc3c(-c4ccc5ccccc5c4)cccc3c2)cc1]

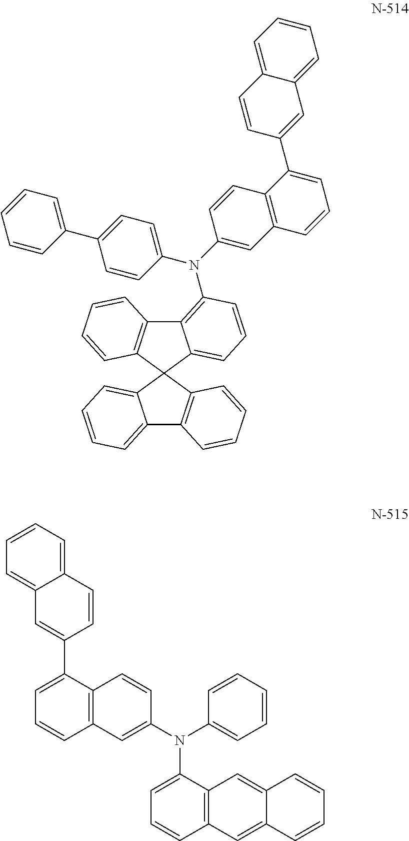 [SMILES: c1ccc(-c2ccc(N(c3ccc4c(-c5ccc6ccccc6c5)cccc4c3)c3cccc4c3-c3ccccc3C43c4ccccc4-c4ccccc43)cc2)cc1 ; c1ccc(N(c2ccc3c(-c4ccc5ccccc5c4)cccc3c2)c2cccc3cc4ccccc4cc23)cc1]

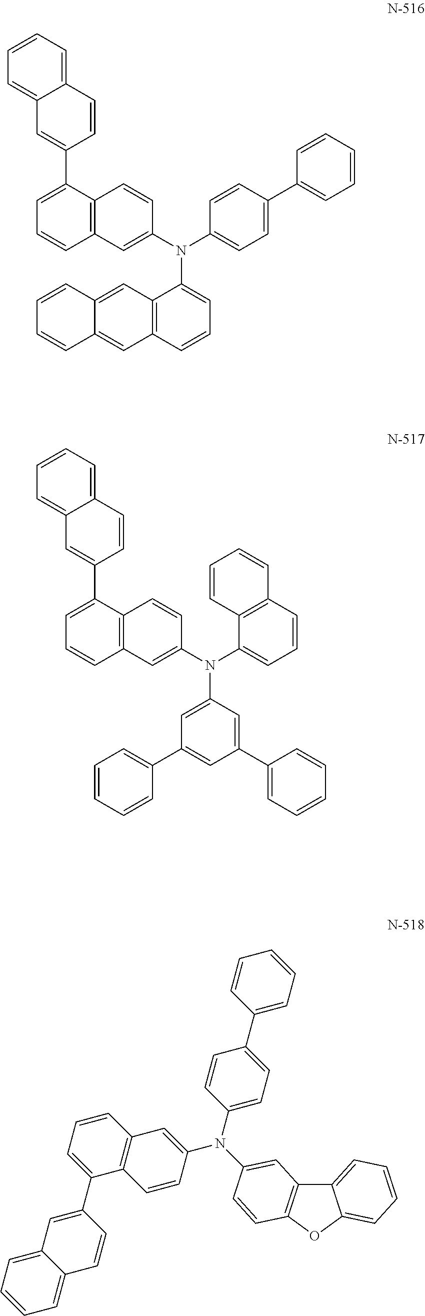 [SMILES: c1ccc(-c2ccc(N(c3ccc4c(-c5ccc6ccccc6c5)cccc4c3)c3cccc4cc5ccccc5cc34)cc2)cc1 ; c1ccc(-c2cc(-c3ccccc3)cc(N(c3ccc4c(-c5ccc6ccccc6c5)cccc4c3)c3cccc4ccccc34)c2)cc1 ; c1ccc(-c2ccc(N(c3ccc4c(-c5ccc6ccccc6c5)cccc4c3)c3ccc4oc5ccccc5c4c3)cc2)cc1]

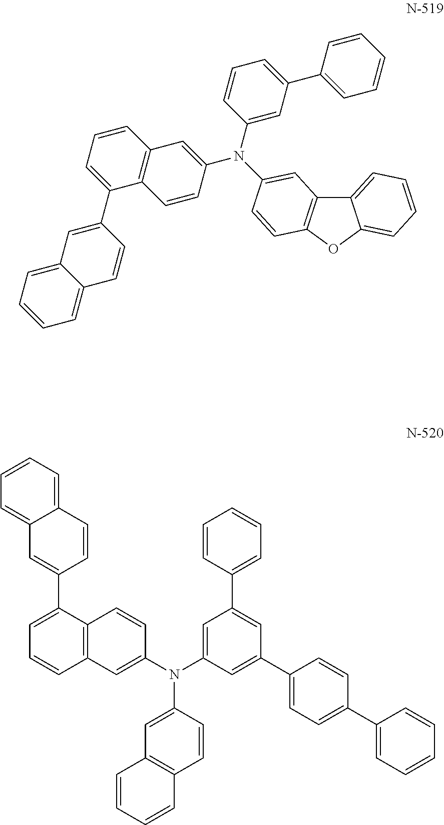 [SMILES: c1ccc(-c2cccc(N(c3ccc4c(-c5ccc6ccccc6c5)cccc4c3)c3ccc4oc5ccccc5c4c3)c2)cc1 ; c1ccc(-c2ccc(-c3cc(-c4ccccc4)cc(N(c4ccc5ccccc5c4)c4ccc5c(-c6ccc7ccccc7c6)cccc5c4)c3)cc2)cc1]

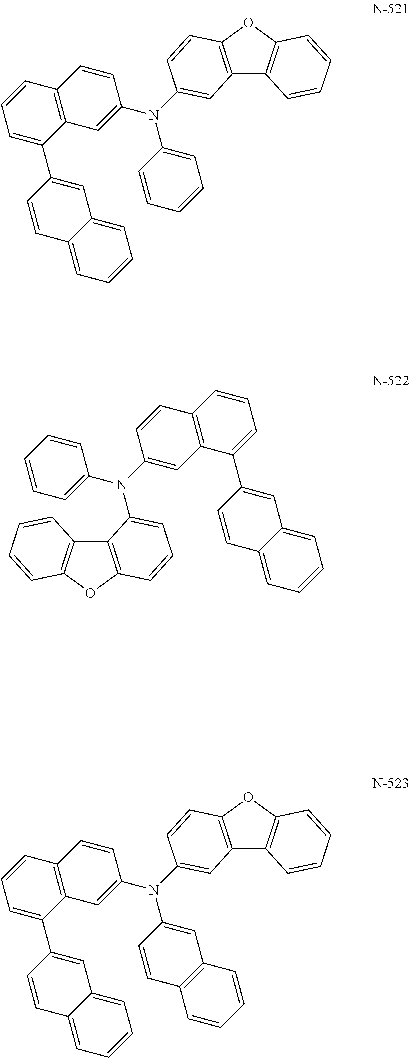 [SMILES: c1ccc(N(c2ccc3cccc(-c4ccc5ccccc5c4)c3c2)c2ccc3oc4ccccc4c3c2)cc1 ; c1ccc(N(c2ccc3cccc(-c4ccc5ccccc5c4)c3c2)c2cccc3oc4ccccc4c23)cc1 ; c1ccc2cc(-c3cccc4ccc(N(c5ccc6ccccc6c5)c5ccc6oc7ccccc7c6c5)cc34)ccc2c1]

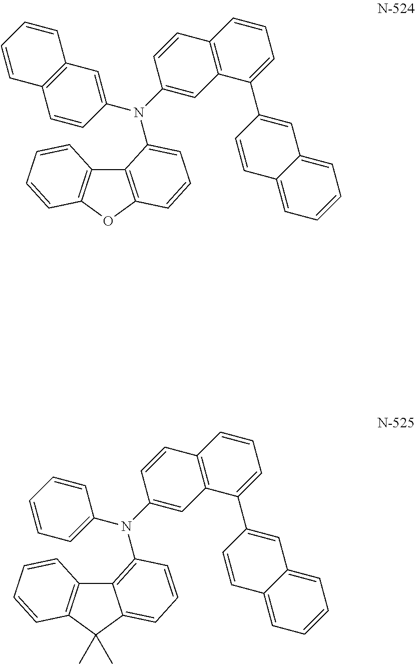 [SMILES: c1ccc2cc(-c3cccc4ccc(N(c5ccc6ccccc6c5)c5cccc6oc7ccccc7c56)cc34)ccc2c1 ; CC1(C)c2ccccc2-c2c(N(c3ccccc3)c3ccc4cccc(-c5ccc6ccccc6c5)c4c3)cccc21]

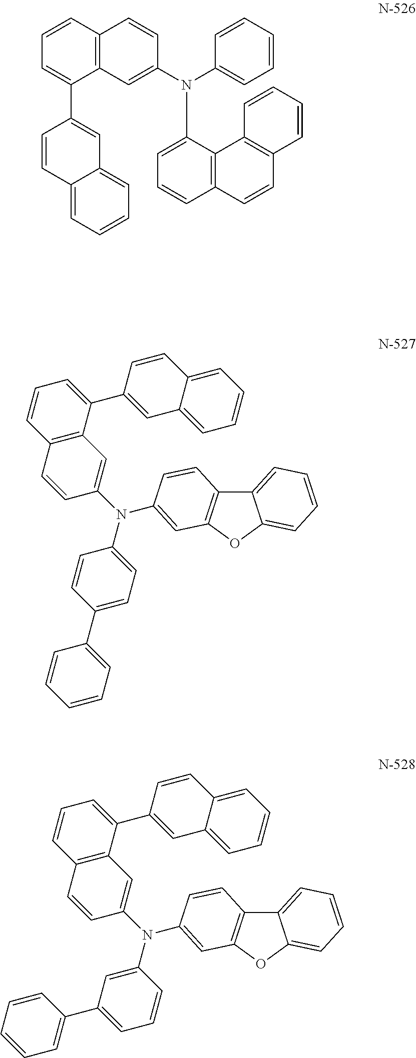 [SMILES: c1ccc(N(c2ccc3cccc(-c4ccc5ccccc5c4)c3c2)c2cccc3ccc4ccccc4c23)cc1 ; c1ccc(-c2ccc(N(c3ccc4c(c3)oc3ccccc34)c3ccc4cccc(-c5ccc6ccccc6c5)c4c3)cc2)cc1 ; c1ccc(-c2cccc(N(c3ccc4c(c3)oc3ccccc34)c3ccc4cccc(-c5ccc6ccccc6c5)c4c3)c2)cc1]

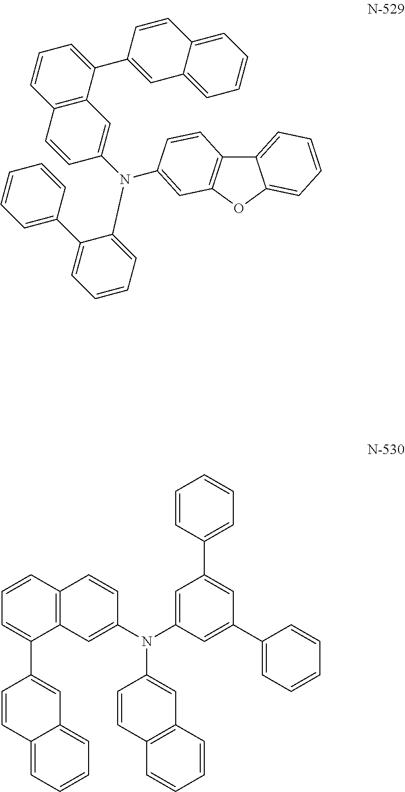 [SMILES: c1ccc(-c2ccccc2N(c2ccc3c(c2)oc2ccccc23)c2ccc3cccc(-c4ccc5ccccc5c4)c3c2)cc1 ; c1ccc(-c2cc(-c3ccccc3)cc(N(c3ccc4ccccc4c3)c3ccc4cccc(-c5ccc6ccccc6c5)c4c3)c2)cc1]

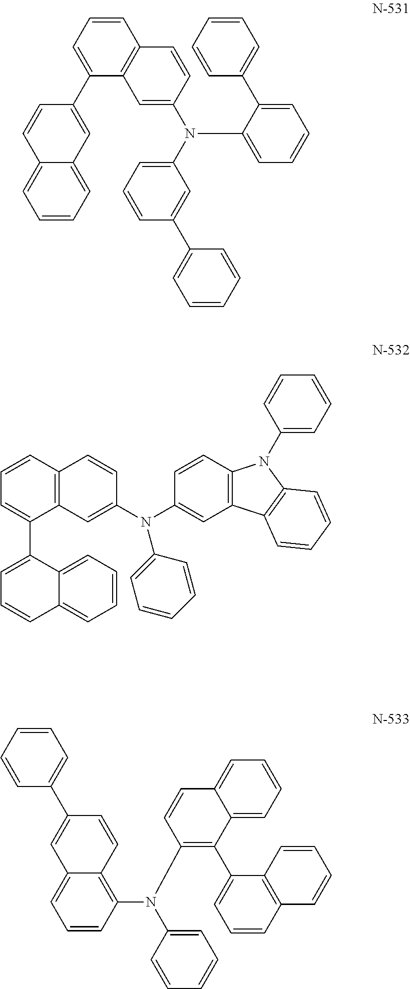 [SMILES: c1ccc(-c2cccc(N(c3ccc4cccc(-c5ccc6ccccc6c5)c4c3)c3ccccc3-c3ccccc3)c2)cc1 ; c1ccc(N(c2ccc3cccc(-c4cccc5ccccc45)c3c2)c2ccc3c(c2)c2ccccc2n3-c2ccccc2)cc1 ; c1ccc(-c2ccc3c(N(c4ccccc4)c4ccc5ccccc5c4-c4cccc5ccccc45)cccc3c2)cc1]

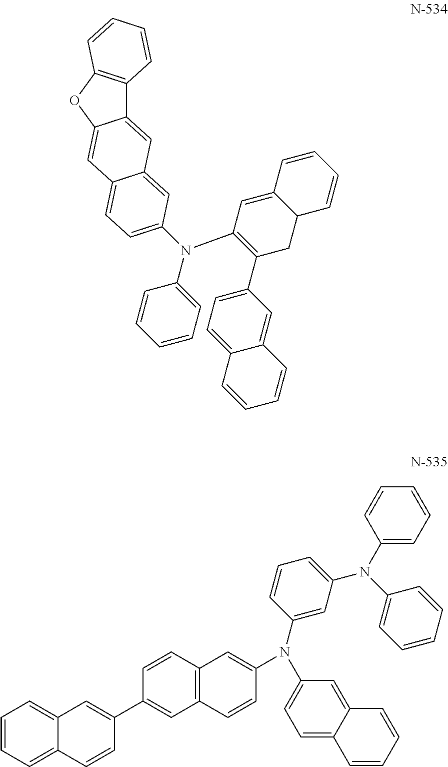 [SMILES: C1=CC2=CC(N(c3ccccc3)c3ccc4cc5oc6ccccc6c5cc4c3)=C(c3ccc4ccccc4c3)CC2C=C1 ; c1ccc(N(c2ccccc2)c2cccc(N(c3ccc4ccccc4c3)c3ccc4cc(-c5ccc6ccccc6c5)ccc4c3)c2)cc1]

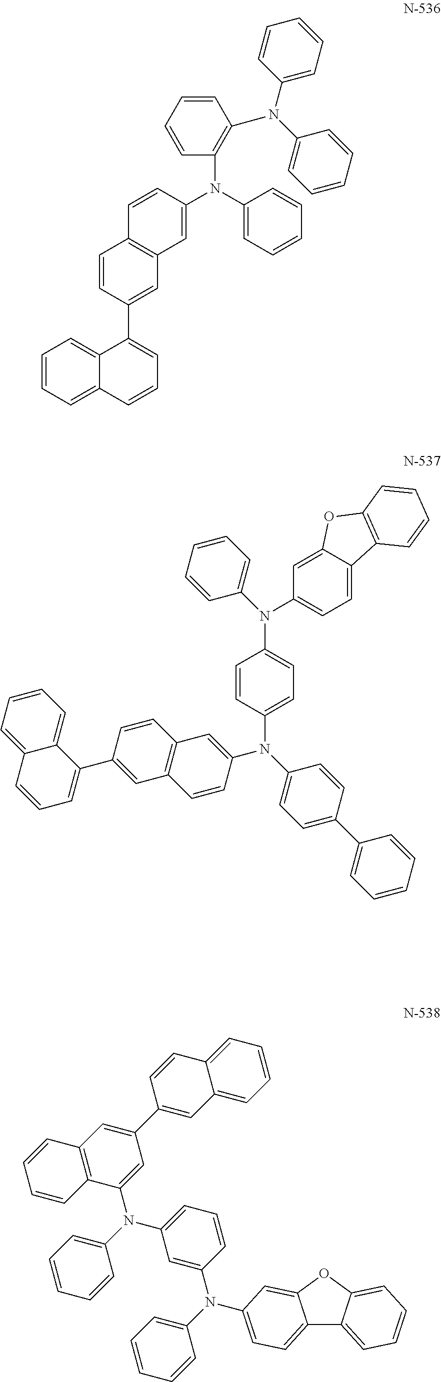 [SMILES: c1ccc(N(c2ccccc2)c2ccccc2N(c2ccccc2)c2ccc3ccc(-c4cccc5ccccc45)cc3c2)cc1 ; c1ccc(-c2ccc(N(c3ccc(N(c4ccccc4)c4ccc5c(c4)oc4ccccc45)cc3)c3ccc4cc(-c5cccc6ccccc56)ccc4c3)cc2)cc1 ; c1ccc(N(c2cccc(N(c3ccccc3)c3cc(-c4ccc5ccccc5c4)cc4ccccc34)c2)c2ccc3c(c2)oc2ccccc23)cc1]

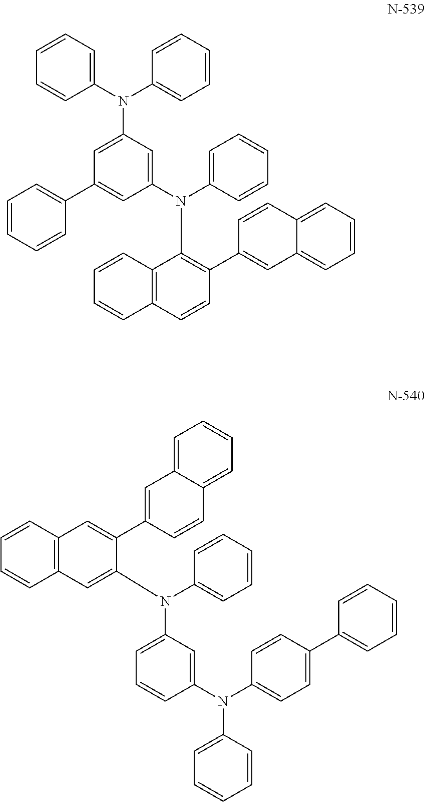 [SMILES: c1ccc(-c2cc(N(c3ccccc3)c3ccccc3)cc(N(c3ccccc3)c3c(-c4ccc5ccccc5c4)ccc4ccccc34)c2)cc1 ; c1ccc(-c2ccc(N(c3ccccc3)c3cccc(N(c4ccccc4)c4cc5ccccc5cc4-c4ccc5ccccc5c4)c3)cc2)cc1]

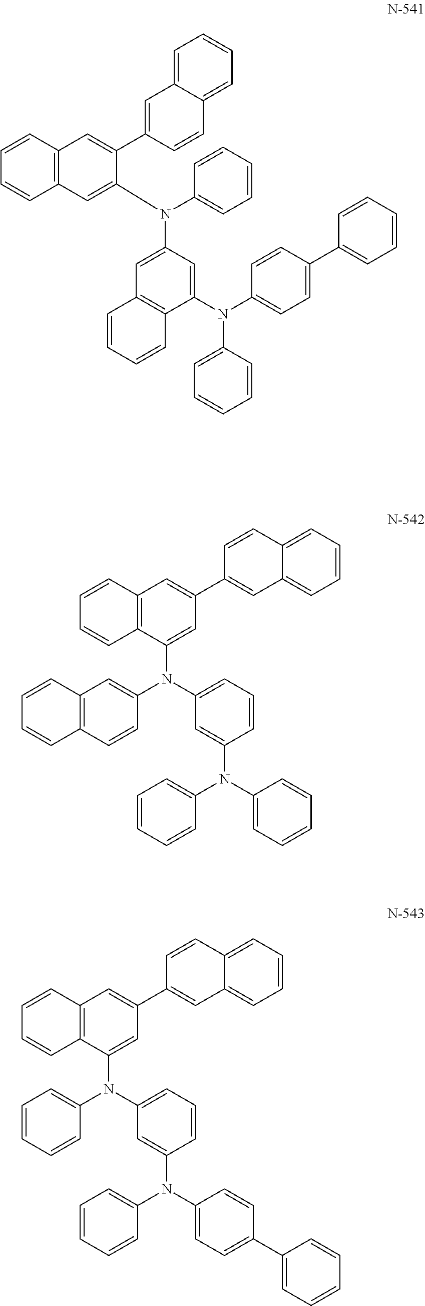 [SMILES: c1ccc(-c2ccc(N(c3ccccc3)c3cc(N(c4ccccc4)c4cc5ccccc5cc4-c4ccc5ccccc5c4)cc4ccccc34)cc2)cc1 ; c1ccc(N(c2ccccc2)c2cccc(N(c3ccc4ccccc4c3)c3cc(-c4ccc5ccccc5c4)cc4ccccc34)c2)cc1 ; c1ccc(-c2ccc(N(c3ccccc3)c3cccc(N(c4ccccc4)c4cc(-c5ccc6ccccc6c5)cc5ccccc45)c3)cc2)cc1]

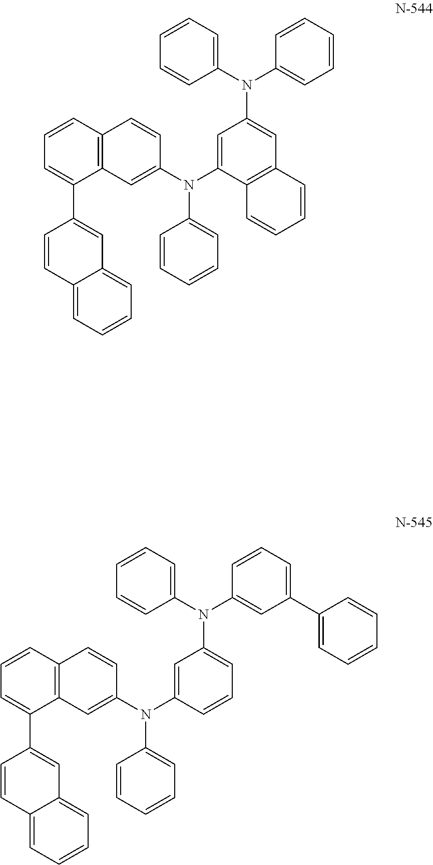 [SMILES: c1ccc(N(c2ccccc2)c2cc(N(c3ccccc3)c3ccc4cccc(-c5ccc6ccccc6c5)c4c3)c3ccccc3c2)cc1 ; c1ccc(-c2cccc(N(c3ccccc3)c3cccc(N(c4ccccc4)c4ccc5cccc(-c6ccc7ccccc7c6)c5c4)c3)c2)cc1]

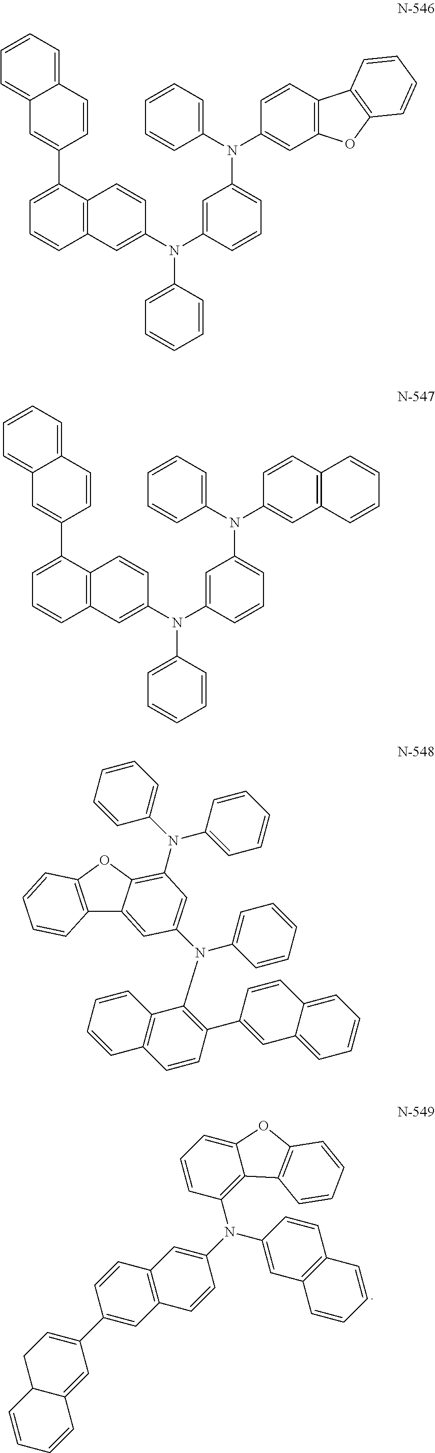 [SMILES: c1ccc(N(c2cccc(N(c3ccccc3)c3ccc4c(c3)oc3ccccc34)c2)c2ccc3c(-c4ccc5ccccc5c4)cccc3c2)cc1 ; c1ccc(N(c2cccc(N(c3ccccc3)c3ccc4c(-c5ccc6ccccc6c5)cccc4c3)c2)c2ccc3ccccc3c2)cc1 ; c1ccc(N(c2cc(N(c3ccccc3)c3ccccc3)c3oc4ccccc4c3c2)c2c(-c3ccc4ccccc4c3)ccc3ccccc23)cc1 ; C1=CC2=CC(c3ccc4cc(N(c5ccc6ccccc6c5)c5cccc6oc7ccccc7c56)ccc4c3)=CCC2C=C1]

11. An organic electroluminescent device, wherein the organic electroluminescent device comprises a cathode, an anode, and an organic layer arranged between the cathode and the anode; wherein the organic layer comprises the organic electroluminescent compound according to claim 1.

12. The organic electroluminescent device according to claim 11, wherein the organic layer comprises a hole transport layer, and the hole transport layer comprises the organic electroluminescent compound.

13. The organic electroluminescent device according to claim 11, wherein the organic layer comprises a light emitting layer, and the light emitting layer comprises the organic electroluminescent compound.

14. A method for manufacturing fiber optic devices, lighting devices, electronic photographic photosensitive devices, photoelectric converters, organic solar cells, switching element devices, organic luminescent field-effect transistors, image sensors, and dye lasers, comprising using the organic electroluminescent device according to claim 11 as an element.

15. An organic electroluminescent device, wherein the organic electroluminescent device comprises a cathode, an anode, and an organic layer arranged between the cathode and the anode; wherein the organic layer comprises the organic electroluminescent compound according to claim 2.

16. An organic electroluminescent device, wherein the organic electroluminescent device comprises a cathode, an anode, and an organic layer arranged between the cathode and the anode; wherein the organic layer comprises the organic electroluminescent compound according to claim 3.

17. An organic electroluminescent device, wherein the organic electroluminescent device comprises a cathode, an anode, and an organic layer arranged between the cathode and the anode; wherein the organic layer comprises the organic electroluminescent compound according to claim 4.

18. An organic electroluminescent device, wherein the organic electroluminescent device comprises a cathode, an anode, and an organic layer arranged between the cathode and the anode; wherein the organic layer comprises the organic electroluminescent compound according to claim 5.

19. An organic electroluminescent device, wherein the organic electroluminescent device comprises a cathode, an anode, and an organic layer arranged between the cathode and the anode; wherein the organic layer comprises the organic electroluminescent compound according to claim 6.

20. An organic electroluminescent device, wherein the organic electroluminescent device comprises a cathode, an anode, and an organic layer arranged between the cathode and the anode; wherein the organic layer comprises the organic electroluminescent compound according to claim 7.
